(19) United States
(12) Patent Application Publication
(10) Pub. No.: US 2004/0127492 A1
(43) Pub. Date: Jul. 1, 2004

(54) Cyclic pyrazoles for the inhibition of mitogen activated protein kinase-activated protein kinase-2

(75) Inventors: Michael L. Vazquez, Ballwin, MO (US); Suzanne Metz, Chesterfield, MO (US); Matthew J. Graneto, Chesterfield, MO (US); Cathleen E. Hanau, Chesterfield, MO (US); Serena Marie Mershon, St. Louis, MO (US); Susan J. Hartmann, Kirkwood, MO (US); David B. Reitz, Chesterfield, MO (US); Gennadiy Poda, Chesterfield, MO (US); Len F. Lee, St. Charles, MO (US); Thao D. Perry, San Jose, CA (US); Emily J. Reinhard, Ridgewood, NJ (US); Leslie Pugh, San Diego, CA (US)

Correspondence Address: Charles E. Dunlap, Keenan Building, Third Floor, 1330 Lady Street, Columbia, SC, 29201, US

(73) Assignee: Pharmacia Corporation, St. Louis, MO

(21) Appl. No.: 10/369,055

(22) Filed: Feb. 18, 2003

Related U.S. Application Data
(60) Provisional application No. 60/357,918, filed on Feb. 19, 2002.

(51) Int. Cl. A61K 31/542; A61K 31/538; A61K 31/53; A61K 31/519; A61K 31/502; A61K 31/498; A61K 31/4745; A61K 31/4162; C07D 487/02; C07D 491/02; C07D 498/02

(52) U.S. Cl. 514/224.2; 514/241; 514/230.5; 514/262.1; 514/248; 514/249; 514/303; 514/406; 544/14; 544/105; 544/184; 544/236; 544/262; 544/350; 546/119; 548/360.1

(57) ABSTRACT

Cyclic pyrazole compounds are described which inhibit mitogen activated protein kinase-activated protein kinase-2 (MK-2). Methods of making such compounds are described, as well as a method of using them for the inhibition of MK-2 in a subject in need of such inhibition, where the method involves administering to the subject an MK-2 inhibiting compound of the present invention. Pharmaceutical compositions and kits which contain the present MK-2 inhibiting compounds are also described.

### CROSS-REFERENCE TO RELATED PATENTS AND PATENT APPLICATIONS

This application claims priority to U.S. Provisional Application Serial No. 60/357,918, filed Feb. 19, 2002, which is hereby incorporated by reference herein in its entirety.

### BACKGROUND OF THE INVENTION

(1) Field of the Invention

The present invention relates to cyclic pyrazole compounds which inhibit mitogen-activated protein kinase-activated protein kinase-2 (MAPKAP-2, or MK-2), and also to methods of using such compounds to inhibit MK-2 and for the prevention and treatment of TNFα mediated diseases or disorders in subjects that are in need of such prevention and/or treatment.

(2) Description of the Related Art

Mitogen-activated protein kinases (MAPKs) are members of conserved signal transduction pathways that activate transcription factors, translation factors and other target molecules in response to a variety of extracellular signals. MAPKs are activated by phosphorylation at a dual phosphorylation motif with the sequence Thr-X-Tyr by mitogen-activated protein kinase kinases (MAPKKs). In higher eukaryotes, the physiological role of MAPK signaling has been correlated with cellular events such as proliferation, oncogenesis, development and differentiation. Accordingly, the ability to regulate signal transduction via these pathways could lead to the development of treatments and preventive therapies for human diseases associated with MAPK signaling, such as inflammatory diseases, autoimmune diseases and cancer.

In mammalian cells, three parallel MAPK pathways have been described. The best characterized pathway leads to the activation of the extracellular-signal-regulated kinase (ERK). Less well understood are the signal transduction pathways leading to the activation of the cJun N-terminal kinase (JNK) and the p38 MAPK. See, e.g., Davis, Trends Biochem. Sci. 19:470-473 (1994); Cano, et al., Trends Biochem. Sci. 20:117-122(1995).

The p38 MAPK pathway is potentially activated by a wide variety of stresses and cellular insults. These stresses and cellular insults include heat shock, UV irradiation, inflammatory cytokines (such as TNF and IL-1), tunicamycin, chemotherapeutic drugs (i.e., cisplatinum), anisomycin, sorbitol/hyperosmolarity, gamma irradiation, sodium arsenite, and ischaemia. See, Ono, K., et al, Cellular Signalling 12,1-13 (2000). Activation of the p38 pathway is involved in (1) production of proinflammatory cytokines, such as TNF-α; (2) induction of enzymes, such as Cox-2; (3) expression of an intracellular enzyme, such as iNOS, which plays an important role in the regulation of oxidation; (4) induction of adherent proteins, such as VCAM-1 and many other inflammatory-related molecules. Furthermore, the p38 pathway functions as a regulator in the proliferation and differentiation of cells of the immune system. See, Ono, K., et al., Id. at 7.

The p38 kinase is an upstream kinase of mitogen-activated protein kinase-activated protein kinase-2 (MAPKAP kinase-2 or MK-2). (See, Freshney, N. W., et al., J. Cell, 78:1039-1049 (1994)). MK-2 is a protein that appears to be predominantly regulated by p38 in cells. Indeed, MK-2 was the first substrate of p38 α to be identified. For example, in vitro phosphorylation of MK-2 by p38 α activates MK-2. The substrates that MK-2 acts upon, in turn, include heat shock protein 27, lymphocyte-specific protein 1.(LAP1), cAMP response element-binding protein (CREB), ATF1, serum response factor (SRF), and tyrosine hydroxylase. The substrate of MK-2 that has been best characterized is small heat shock protein 27 (hsp27).

The role of the p38 pathway in inflammatory-related diseases has been studied in several animal models. The pyridinyl imidazole compound SB203580 has been shown to be a specific inhibitor of p38 in vivo, and also has been shown to inhibit activation of MK-2, (See, Rouse, J., et al, Cell, 78:1027-1037 (1994); Cuenda, A., et al, Biochem. J., 333:11-15 (1998)), as well as a MAP kinase homologue termed reactivating kinase (RK). (See, Cuenda, A., et al., FEBS Lett., 364(2):229-233 (1995)). Inhibition of p38 by SB203580 can reduce mortality in a murine model of endotoxin-induced shock and inhibit the development of mouse collagen-induced arthritis and rat adjuvant arthritis. See, e.g., Badger, A. M., et al., J. Pharmacol Exp. Ther., 279:1453-1461 (1996). Another p38 inhibitor that has been utilized in an animal model that is believed to be more potent than SB203580 in its inhibitory effect on p38 is SB 220025. A recent animal study has demonstrated that SB 220025 caused a significant dose-dependent decrease in vascular density of granulomas in laboratory rats. (Jackson, J. R., et al, J. Pharmacol. Exp. Ther., 284:687-692 (1998)). The results of these animal studies indicated that p38, or the components of the p38 pathway, Can be useful therapeutic targets for the prevention or treatment of inflammatory disease.

Due to its integral role in the p38 signaling pathway, MK-2 has been used as a monitor for measuring the level of activation in the pathway. Because of its downstream location in the pathway, relative to p38, MK-2 has been measured as a more convenient, albeit indirect, method of assessing p38 activation. However, so far, research efforts exploring therapeutic strategies associated with the modulation of this pathway have focused mainly on the inhibition of p38 kinase.

Several compounds that inhibit the activity of p38 kinase have been described in U.S. Pat. Nos. 6,046,208, 6,251,914, and 6,335,340. These compounds have been suggested to be useful for the treatment of CSBP/RK/p38 kinase mediated disease. Commercial efforts to apply p38 inhibitors have centered around two p38 inhibitors, the pyridinylimidazole inhibitor SKF 86002, and the 2,4,5 triaryl imidazole inhibitor SB203580. See, Lee, J. C., et al, Immunopharmacology 47,185-192 (2000). Compounds possessing a similar structure have also been investigated as potential p38 inhibitors. Indeed, p38 MSP kinase's role in various disease states has been elucidated through the use of inhibitors.

Kotlyarov, A. et al, in Nat Cell Biol., 1(2):94-97 (1999) introduced a targeted mutation into a mouse MK-2 gene, resulting in MK-2-deficient mice. It was shown that mice lacking MK-2 possessed increased stress resistance and survived LPS-induced endotoxic shock better than MK-2+mice. The authors concluded that MK-2 was an essential component in the inflammatory response that regulates biosynthesis of TNFα at a post-transcriptional level. More recently, Lehner, M. D., et al, in J. Immunol., 168(9):4667-4673 (2002), reported that MK-2-deficient mice showed increased susceptibility to Listeria monocytogenes infection, and concluded that MK-2 had an essential role in host defense against intracellular bacteria, probably via regulation of TNF and IFN-gamma production required for activation of antibacterial effector mechanisms.

The location of MK-2 in the p38 signaling pathway at a point that is downstream of p38 offers the potential that MK-2 could act as a focal point for modulating the pathway without affecting as many substrates as would the regulation of an enzyme further upstream in the signaling cascade—such as p38 MAP kinase.

Accordingly, it would be useful to provide compounds and methods that could serve to modulate the activity of MK-2—in particular, to act as inhibitors of MK-2 activity. Such compounds and methods would be useful for the provision of benefits similar to p38 MAP kinase inhibitors, which benefits include the prevention and treatment of diseases and disorders that are mediated by TNFα. It would be even more useful to provide MK-2 inhibitors having improved potency and reduced undesirable side effects, relative to p38 inhibitors.

### SUMMARY OF THE INVENTION

Briefly therefore, the present invention is directed to a novel a novel cyclic pyrazole MK-2 inhibiting compound having the structure:

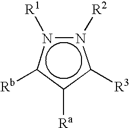

where:

R^{a} and R^{b} join to form a ring structure selected from the group

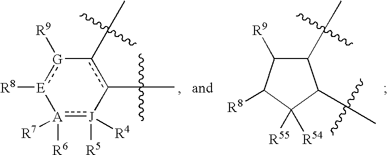

consisting of:

A, E, G and J are carbon, or optionally, one of A, E, G and J is selected from nitrogen, sulfur, or oxygen;

when A, E, G, or J is oxygen or sulfur, no substituent groups are bonded to the oxygen or the sulfur;

when A, E, G, or J is nitrogen, the nitrogen is optionally unsubstituted, or is substituted with R⁹;

each of R¹, R², R⁴ and R⁶ is optionally absent, or is independently selected from the R³ groups;

R³, R⁵, R⁷, R⁸, R⁹, R⁴⁰, R⁵⁴ and R⁵⁵ are each independently selected from —H, C₁-C₆ alkyl, C₂-C₆ alkenyl, C₂-C₆ alkynyl, nitro C₁-C₆ alkyl, azido C₁-C₆ alkyl, amino, NHR¹⁰, NR¹¹R¹², C₁-C₆ alkyl-NHR¹⁰, C₁-C₆ -alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁵;

R¹⁰, R¹¹, are each independently selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, CO₂R¹⁹, COR²⁰, CONHR¹⁹, CON(R¹⁹)₂, SR¹⁸, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, arylaryl, arylheteroaryl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹², R¹³ are each independently selected from —H, C₁-C₆ alkyl, C₂-C₆ alkenyl, C₂-C₆ alkynyl, C₁-C₆ alkyl-NHR¹⁶, C₁-C₆ alkyl-NR¹⁶R¹⁷, C₁-C₄ alkyl-OR¹⁸, aryl, heteroaryl, heterocyclyl, CO₂R¹⁹, COR²⁰, CONHR¹⁹, CON(R¹⁹)₂, SOR²⁰, SO₂R²⁰, C₁-C₆ alkyl CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁴ is selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, SR¹⁸, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁵ is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁶ and R¹⁷ are each independently selected from —H, C₁-C₆ alkyl, C₂-C₆ alkenyl, C₂-C₆ alkynyl, amino, C₁-C₆ alkyl-NHR²², C₁-C₆ alkyl-NR²²R²³, C₁, C₄ alkyl-OR²⁴, aryl, heteroaryl, heterocyclyl, CO₂R²⁵, COR²⁶, CONHR²⁵, CON(R²⁵)₂, SOR²⁶, SO₂R²⁶, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R¹⁸, R¹⁹ are each independently selected from —H, C₁-C₆ alkyl, C₂-C₆ alkenyl, C₂-C₆ alkynyl, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁷, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²⁰ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, O—R²⁴, C₁-C₄ alkyl-OR²⁴, aryl, heteroaryl, heterocyclyl, SR²⁴, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, arylalkoxyalkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²¹ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, nitro, cyano, O—R²⁴, C₁-C₄ alkyl-OR²⁴, aryl, heteroaryl, heterocyclyl, COR²⁶, SR²⁴, SOR²⁶, SO₂R²⁶, C₁-C₆ alkyl-COR²⁶, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²² and R²³ are each independently selected from —H, C₁-C₆ alkyl, C₂-C₆ alkenyl, C₂-C₆ alkynyl, amino, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁴ and R²⁵ are each independently selected from —H, C₁-C₆ alkyl, C₂-C₆ alkenyl, C₂-C₆ alkynyl, C₁-C₆ alkyl-NHR²⁹, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁶ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, O—R³⁰, C₁-C₄ alkyl-OR³⁰, aryl, heteroaryl, heterocyclyl, SR³⁰, C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁷ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, nitro, cyano, O—R³⁰, C₁-C₄ alkyl-OR³⁰, aryl, heteroaryl, heterocyclyl, COR³², SR³⁰, SOR³², SO₂R³², C₁-C₆ alkyl-COR³², C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic-cycloalkyl are optionally substituted with one or more of the groups defined by R³³;

R²⁸ and R²⁹ are each independently selected from —H, C₁-C₆ alkyl, C₂-C₆ alkenyl, C₂-C₆ alkynyl, amino, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁰ and R³¹ are each independently selected from —H, C₁-C₆ alkyl, C₂-C₆ alkenyl, C₂-C₆ alkynyl, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³² is selected from —H, C₁-C₆ alkyl, C₂-C₆ alkenyl, C₂-C₆ alkynyl, amino, NHR³⁴, NR³⁴R³⁵, C₁-C₆ alkyl-NHR³⁴, C₁-C₆ alkyl-NR³⁴R³⁵, O—R³⁶, C₁-C₄ alkyl-OR³⁶, aryl, heteroaryl, heterocyclyl, SR³⁶, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³³ is selected from —H, C₁-C₆ alkyl, C₂-C₆ alkenyl, C₂-C₆ alkynyl, amino, NHR³⁴, NR³⁴R³⁵, C₁-C₆ alkyl-NHR³¹, C₁-C₆ alkyl-NR³⁴R³⁵, nitro, cyano, O—R³⁶, C₁-C₄ alkyl-OR³⁶, aryl, heteroaryl, heterocyclyl, COR³⁸, SR³⁵, SOR³⁸, SO₂R³⁸, C₁-C₆ alkyl-COR³⁸, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁴, R³⁵, R³⁶ and R³⁷ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁸ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁹ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, arylalkenyl, heterocyclylalkenyl, arylheterocyclylalkenyl, heterocyclylarylalkenyl, or heteroarylalkyl; and

R² and R³, or R⁸ and R⁹, optionally join to form a saturated or unsaturated ring of 5, 6, 7, or 8 atoms, where the atoms in the ring are independently selected from CR⁴⁰, C(R⁴⁰)₂, C═O, N, NR⁴⁰, O, S, C═S, S═O, or SO₂.

The present invention is also directed to a novel cyclic pyrazole MK-2 inhibiting compound having the structure:

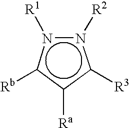

where:

R^{a} and R^{b} join to form a ring structure selected from the group consisting of:

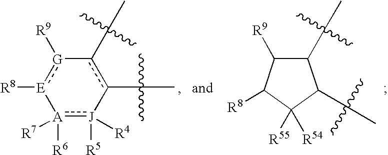

A, E, G and J are carbon, or optionally, one of A, E, G and J is selected from nitrogen, sulfur, or oxygen;

when A, E, G, or J is oxygen or sulfur, no substituent groups are bonded to the oxygen or the sulfur;

when A, E, G, or J is nitrogen, the nitrogen is optionally unsubstituted, or is substituted with R⁹;

each of R¹, R², R⁴ and R⁶ is optionally absent, or is independently selected from the R³ groups;

R³, R⁵, R⁷, R⁸, R⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴ and R⁵⁵ are each independently selected from —H, C₁-C₆ alkyl, C₂-C₆ alkenyl, C₂-C₆ alkynyl, nitro C₁-C₆ alkyl, azido C₁-C₆ alkyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁵, or R⁸ and R⁹ join to form a ring structure selected from:

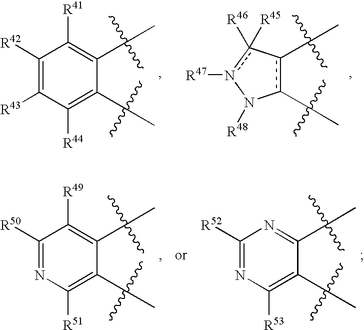

R¹⁰, R¹¹, are each independently selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, CO₂R¹⁹, COR²⁰, CONHR¹⁹, CON(R¹⁹)₂, SR¹⁸, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, arylaryl, arylheteroaryl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹², R¹³ are each independently selected from —H, C₁-C₆ alkyl, C₂-C₆ alkenyl, C₂-C₆ alkynyl, C₁-C₆ alkyl-NHR¹⁶, C₁-C₆ alkyl-NR¹⁶R¹⁷, C₁-C₄ alkyl-OR¹⁸, aryl, heteroaryl, heterocyclyl, CO₂R¹⁹, COR²⁰, CONHR¹⁹, CON(R¹⁹)₂, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁴ is selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, SR¹⁸, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁵ is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁶ and R¹⁷ are each independently selected from —H, C₁-C₆ alkyl, C₂-C₆ alkenyl, C₂-C₆ alkynyl, amino, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁶, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R¹⁸, R¹⁹ are each independently selected from —H, C₁-C₆ alkyl, C₂-C₆ alkenyl, C₂-C₆ alkynyl, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁷, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²⁰ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, O—R²⁴, C₁-C₄ alkyl-OR²⁴, aryl, heteroaryl, heterocyclyl, SR²⁴, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, arylalkoxyalkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²¹ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, nitro, cyano, O—R²⁴, C₁-C₄ alkyl-OR²⁴, aryl, heteroaryl, heterocyclyl, COR²⁶, SR²⁴, SOR²⁶, SO₂R²⁶, C₁-C₆ alkyl-COR²⁶, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²² and R²³ are each independently selected from —H, C₁-C₆ alkyl, C₂-C₆ alkenyl, C₂-C₆ alkynyl, amino, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁴ and R²⁵ are each independently selected from —H, C₁-C₆ alkyl, C₂-C₆ alkenyl, C₂-C₆ alkynyl, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁶ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, O—R³⁰, C₁-C₄ alkyl-OR³⁰, aryl, heteroaryl, heterocyclyl, SR³⁰, C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁷ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, nitro, cyano, O—R³⁰, C₁-C₄ alkyl-OR³⁰, aryl, heteroaryl, heterocyclyl, COR³², SR³⁰, SOR³², SO₂R³², C₁-C₆ alkyl-COR³², C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁸ and R²⁹ are each independently selected from —H, C₁-C₆ alkyl, C₂-C₆ alkenyl, C₂-C₆ alkynyl, amino, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁰ and R³¹ are each independently selected from —H, C₁-C₆ alkyl, C₂-C₆ alkenyl, C₂-C₆ alkynyl, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸; C₁-C₆ alkyl-SO₂R³⁸, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³² is selected from —H, C₁-C₆ alkyl, C₂-C₆ alkenyl, C₂-C₆ alkynyl, amino, NHR³⁴, NR³⁴R³⁵, C₁-C₆ alkyl-NHR³⁴, C₁-C₆ alkyl-NR³⁴R³⁵, O—R³⁶, C₁-C₄ alkyl-OR³⁶, aryl, heteroaryl, heterocyclyl, SR³⁶, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³³ is selected from —H, C₁-C₆ alkyl, C₂-C₆ alkenyl, C₂-C₆ alkynyl, amino, NHR³⁴, NR³⁴R³⁵, C₁-C₆ alkyl-NHR³¹, C₁-C₆ alkyl-NR³⁴R³⁵, nitro, cyano, O—R³⁶, C₁-C₄ alkyl-OR³⁶, aryl, heteroaryl, heterocyclyl, COR³⁸, SR³⁵, SOR³⁸, SO₂R³⁸, C₁-C₆ alkyl-COR³⁸, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁴, R³⁵, R³⁶ and R³⁷ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³¹ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁹ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, arylalkenyl, heterocyclylalkenyl, arylheterocyclylalkenyl, heterocyclylarylalkenyl, or heteroarylalkyl; and

R² and R³ optionally join to form a saturated or unsaturated ring of 5, 6, 7, or 8 atoms, where the atoms in the ring are independently selected from CR⁴⁰, C(R⁴⁰)₂, C═O, N, NR⁴⁰, O, S, C═S, S═O, or SO₂.

The present invention is also directed to a novel method of inhibiting mitogen activated protein kinase-activated protein kinase-2 in a subject in need of such inhibition, the method comprising administering to the subject an MK-2 inhibiting compound having the structure shown above.

The present invention is also directed to a novel method of preventing or treating a TNFα mediated disease or disorder in a subject in need of such prevention or treatment, the method comprising administering to the subject an effective amount of an MK-2 inhibiting compound having the structure shown above

The present invention is also directed to a novel pharmaceutical composition comprising an MK-2 inhibiting compound comprising a pharmaceutical carrier and an MK-2 inhibiting compound having the structure shown above.

The present invention is also directed to a novel kit comprising a dosage form that includes an MK-2 inhibiting compound in a therapeutically effective amount, the MK-2 inhibiting compound having the structure shown above.

Among the several advantages found to be achieved by the present invention, therefore, may be noted the provision of a method that could serve to modulate the activity of MK-2—in particular, to inhibit MK-2 activity, and the provision of a method for the prevention and treatment of diseases and disorders that are mediated by TNFα.

### BRIEF DESCRIPTION OF THE DRAWINGS

FIG. 1 is a graph showing paw thickness as a function of time from day 0 to day 7 for MK2 (+/+) and MK2 (−/−) mice, which have received serum injection; and

FIG. 2 is a bar chart showing paw thickness at seven days after injection for normal mice, MK2 (+/+) mice receiving serum, MK2 (−/−) mice receiving serum, and MK2 (+/+) mice receiving serum and anti-TNF antibody.

### DETAILED DESCRIPTION OF THE PREFERRED EMBODIMENTS

In accordance with the present invention, it has been discovered that certain cyclic pyrazole compounds can inhibit the activity of MAPKAP kinase-2. Many of these compounds exhibit their inhibitory effect at low concentrations—having in vitro MK-2 inhibition IC₅₀ values of under 10.0 EM, and with some having IC₅₀ values of under about 1 μM, and even as low as about 0.2 μM. Accordingly, these compounds can be potent and effective drugs for use in the inhibition of MK-2, and of special value for administration to subjects where such inhibition would be useful. In particular, these compounds would be useful in methods to prevent or treat diseases and disorders that are mediated by TNFα. For example, they can be used for the prevention or treatment of arthritis.

Compounds that have a high degree of MK-2 inhibiting activity offer advantages in therapeutic uses, because lower amounts of the compounds can be used to obtain a therapeutic benefit than with less active compounds. Such highly active compounds might also offer fewer side effects, and a higher selectivity.

The terms “cyclic pyrazole MK-2 inhibitor”, or “cyclic pyrazole MK-2 inhibiting compound”, which may be used interchangeably herein, embrace certain fused bicyclic or tricyclic compounds wherein one of the rings is a pyrazole, which inhibit mitogen activated protein kinase-activated protein kinase-2, and also include pharmaceutically acceptable salts of those compounds. When the cyclic pyrazole compounds have three fused rings, the ring that is furthest from the pyrazole, Can be a phenyl ring, which, when substituted with an hydroxy group, is termed a phenoxy ring. Cyclic pyrazoles having such a phenoxy ring can be referred to here as “phenoxy pyrazoles”. Other tricyclic fused ring pyrazoles can have as the third ring another pyrazole (and can be referred to herein as pyrazolyl pyrazoles), or a pyridine (which can be referred to herein as pyridyl pyrazoles), or a pyrimidine ring (which can be referred to herein as pyrimidyl pyrazoles). Certain embodiments of the cyclic pyrazole MK-2 inhibitors of the present invention selectively inhibit MK-2 relative to MK-3.

In practice, the selectivity of a cyclic pyrazole MK-2 inhibitor varies depending upon the condition under which the test is performed and on the inhibitors being tested. However, for the purposes of this specification, the selectivity of a cyclic pyrazole MK-2 inhibitor inhibitor can be measured as a ratio of the in vitro or in vivo IC₅₀ value for inhibition of MK-3, divided by the IC₅₀ value for inhibition of MK-2 (IC_{50 MK-3}/IC_{50 MK-2}). As used herein, the term “IC₅₀” refers to the concentration of a compound that is required to produce 50% inhibition of MK-2 or MK-3 activity. An MK-2 selective inhibitor is any inhibitor for which the ratio of IC₅₀ MK-3 to IC_{50 MK-2} is greater than 1. In preferred embodiments, this ratio is greater than 2, more preferably greater than 5, yet more preferably greater than 10, still more preferably greater than 50, and more preferably still, is greater than 100. Such preferred selectivity may indicate an ability to reduce the incidence of side effects incident to the administration of an MK-2 inhibitor to a subject.

Also included within the scope of the present invention are compounds that act as prodrugs of the present cyclic pyrazole MK-2 inhibitors. As used herein in reference to cyclic pyrazole MK-2 inhibitors, the term “prodrug” refers to a chemical compound that can be converted into an active cyclic pyrazole MK-2 inhibitor by metabolic or simple chemical processes within the body of the subject.

Cyclic pyrazole MK-2 inhibiting compounds of the present invention include those having the structure shown in formula I:

Formula I:

A cyclic pyrazole MK-2 inhibiting compound having the structure:

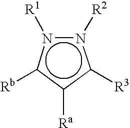

where:

R^{a} and R^{b} join to form a ring structure selected from the group consisting of:

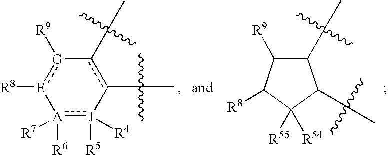

A, E, G and J are carbon, or optionally, one of A, E, G and J is selected from nitrogen, sulfur, or oxygen;

when A, E, G, or J is oxygen or sulfur, no substituent groups are bonded to the oxygen or the sulfur;

when A, E, G, or J is nitrogen, the nitrogen is optionally unsubstituted, or is substituted with R⁹;

each of R¹, R², R⁴ and R⁶ is optionally absent, or is independently selected from the R³ groups;

R³, R⁵, R⁷, R⁸, R⁹, R⁴⁰, R⁵⁴ and R⁵⁵ are each independently selected from —H, C₁-C₆ alkyl, C₂-C₆ alkenyl, C₂-C₆ alkynyl, nitro C₁-C₆ alkyl, azido C₁-C₆ alkyl, amino, NHR¹⁰, NR¹¹R¹², C₁-C₆ alkyl-NHR¹⁰, C₁-C₆-alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁵;

R¹⁰, R¹¹, are each independently selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, CO₂R¹⁹, COR²⁰, CONHR¹⁹, CON(R¹⁹)₂, SR¹⁸, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, arylaryl, arylheteroaryl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹², R¹³ are each independently selected from —H, C₁-C₆ alkyl, C₂-C₆ alkenyl, C₂-C₆ alkynyl, C₁-C₆ alkyl-NHR¹⁶, C₁-C₆ alkyl-NR¹⁶R¹⁷, C₁-C₄ alkyl-OR¹⁸, aryl, heteroaryl, heterocyclyl, CO₂R¹⁹, COR²⁰, CONHR¹⁹, CON(R¹⁹)₂, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁴ is selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, SR¹⁸, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁵ is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁶ and R¹⁷ are each independently selected from —H, C₁-C₆ alkyl, C₂-C₆ alkenyl, C₂-C₆ alkynyl, amino, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁶, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R¹⁸, R¹⁹ are each independently selected from —H, C₁-C₆ alkyl, C₂-C₆ alkenyl, C₂-C₆ alkynyl, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁷, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²⁰ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, O—R²⁴, C₁-C₄ alkyl-OR²⁴, aryl, heteroaryl, heterocyclyl, SR²⁴, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-Ca alkyl-SOR²⁶, C₁-C₆ alkyl-SO₂R²⁶, halo, halo C₁-C₆ alkyl, arylalkoxyalkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²¹ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, nitro, cyano, O—R²⁴, C₁-C₄ alkyl-OR²⁴, aryl, heteroaryl, heterocyclyl, COR²⁶, SR²⁴, SOR²⁶, SO₂R²⁶, C₁-C₆ alkyl-COR²⁶, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²² and R²³ are each independently selected from —H, C₁-C₆ alkyl, C₂-C₆ alkenyl, C₂-C₆ alkynyl, amino, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁴ and R²⁵ are each independently selected from —H, C₁-C₆ alkyl, C₂-C₆ alkenyl, C₂-C₆ alkynyl, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁶ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, O—R³⁰, C₁-C₄ alkyl-OR³⁰, aryl, heteroaryl, heterocyclyl, SR³⁰, C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁷ is selected from —H, C₁-C₆ alkyl, C₂-C₆ alkenyl, C₂-C₆ alkynyl, amino, NHR²⁸, NR²⁵R²⁹, C₁-C₆ alkyl-NHR²⁸, C₁-C₆ alkyl-NR²⁸R²⁹, nitro, cyano, O—R³⁰, C₁-C₄ alkyl-OR³⁰, aryl, heteroaryl, heterocyclyl, COR³², SR³⁰, SOR³², SO₂R³², C₁-C₆ alkyl-COR³², C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁸ and R²⁹ are each independently selected from —H, C₁-C₆ alkyl, C₂-C₆ alkenyl, C₂-C₆ alkynyl, amino, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁰ and R³¹ are each independently selected from —H, C₁-C₆ alkyl, C₂-C₆ alkenyl, C₂-C₆ alkynyl, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁷, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³² is selected from —H, C₁-C₆ alkyl, C₂-C₆ alkenyl, C₂-C₆ alkynyl, amino, NHR³⁴, NR³⁴R³⁵, C₁-C₆ alkyl-NHR³⁴, C₁-C₆ alkyl-NR³⁴R³⁵, O—R³⁶, C₁-C₄ alkyl-OR³⁶, aryl, heteroaryl, heterocyclyl, SR³⁶, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³³ is selected from —H, C₁-C₆ alkyl, C₂-C₆ alkenyl, C₂-C₆ alkynyl, amino, NHR³⁴, NR³⁴R³⁵, C₁-C₆ alkyl-NHR³¹, C₁-C₆ alkyl-NR³⁴R³⁵, nitro, cyano, O—R³⁶, C₁-C₄ alkyl-OR³⁶, aryl, heteroaryl, heterocyclyl, COR³⁸, SR³⁵, SOR³⁸, SO₂R³⁸, C₁-C₆ alkyl-COR³⁸, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁴, R³⁵, R³⁶ and R³⁷ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁸ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁹ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, arylalkenyl, heterocyclylalkenyl, arylheterocyclylalkenyl, heterocyclylarylalkenyl, or heteroarylalkyl; and

R² and R³, or R⁸ and R⁹, optionally join to form a saturated or unsaturated ring of 5, 6, 7, or 8 atoms, where the atoms in the ring are independently selected from CR⁴⁰, C(R⁴⁰)₂, C═O, N, NR⁴⁰, O, S, C═S, S═O, or SO₂.

The meaning of any substituent at any one occurrence in Formula I, or any other general chemical formula herein, is independent of its meaning, or any other substituent's meaning, at any other occurrence, unless specified otherwise.

The term “alkyl” is used, either alone or within other terms such as “haloalkyl” and “alkylsulfonyl”; it embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about five carbon atoms. The number of carbon atoms can also be expressed as “C₁-C₅”, for example. Examples of such radicals include methyl, ethyl, n-propyl, isopropyl, n-butyl, isobutyl, sec-butyl, tert-butyl, pentyl, isoamyl, hexyl, octyl and the, like. The term “alkenyl” refers to an unsaturated, acyclic hydrocarbon radical, linear or branched, in so much as it contains at least one double bond. Unless otherwise noted, such radicals preferably contain from 2 to about 6 carbon atoms, preferably from 2 to about 4 carbon atoms, more preferably from 2 to about 3 carbon atoms. The alkenyl radicals may be optionally substituted with groups as defined below. Examples of suitable alkenyl radicals include propenyl, 2-chloropropylenyl, buten-1yl, isobutenyl, penten-1yl, 2-methylbuten-1-yl, 3-methylbuten-1-yl, hexen-1-yl, 3-hydroxyhexen-1-yl, hepten-1-yl, octen-1-yl, and the like. The term “alkynyl” refers to an unsaturated, acyclic hydrocarbon radical, linear or branched, in so much as it contains one or more triple bonds, such radicals preferably containing 2 to about 6 carbon atoms, more preferably from 2 to about 3 carbon atoms. The alkynyl radicals may be optionally substituted with groups as described below. Examples of suitable alkynyl radicals include ethynyl, proynyl, hydroxypropynyl, butyn-1-yl, butyn-2-yl, pentyn-1-yl, pentyn-2-yl, 4-methoxypentyn-2-yl, 3-methylbutyn-1-yl, hexyl-1-yl, hexyn-2-yl, hexyn-3-yl, 3,3-dimethylbutyn-1-yl radicals, and the like. The term “oxo” means a single double-bonded oxygen. The terms “hydrido”, “—H”, or “hydrogen”, denote a single hydrogen atom (H). This hydrido radical may be attached, for example, to an oxygen atom to form a hydroxyl radical, or two hydrido radicals may be attached to a carbon atom to form a methylene (—CH₂—) radical. The term “halo” means halogens such as fluorine, Chlorine, and bromine or iodine atoms. The term “haloalkyl” embraces radicals wherein any one or more of the alkyl carbon atoms is substituted with halo as de-fined above. Specifically embraced are monohaloalkyl, dihaloalkyl, and polyhaloalkyl radicals. A monohaloalkyl radical, for one example, may have a bromo, Chloro, or a fluoro atom within the radical. Dihalo radicals may have two or more of the same halo atoms or a combination of different halo radicals and polyhaloalkyl radicals may have more than two of the same halo atoms or a combination of different halo radicals. The term “hydroxyalkyl” embraces linear or branched alkyl radicals having one to about ten carbon atoms any one of which may be substituted with one or more hydroxyl radicals. The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and dialkoxyalkyl radicals. The “alkoxy” or “alkoxyalkyl” radicals may be further substituted with one or more halo atoms, such as fluoro, Chloro, or bromo, to provide “haloalkoxy” or “haloalkoxyalkyl” radicals. Examples of “alkoxy” radicals include methoxy, butoxy, and trifluoromethoxy. Terms such as “alkoxy(halo)alkyl”, indicate a molecule having a terminal alkoxy that is bound to an alkyl, which is bonded to the parent molecule, while the alkyl also has a substituent halo group in a non-terminal location. In other words, both the alkoxy and the halo group are substituents of the alkyl chain. The term “aryl”, alone or in combination, means a carbocyclic aromatic system containing one, two, or three rings wherein such rings may be attached together in a pendent manner or may be fused. The term “aryl” embraces aromatic radicals such as phenyl, naphthyl, tetrahydronapthyl, indane, and biphenyl. The term “heterocyclyl” means a saturated or unsaturated mono- or multi-ring carbocycle wherein one or more carbon atoms is replaced by N, S, P, or O. This includes, for example, structures such as:

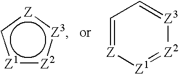

where Z, Z¹, Z², or Z³ is C, S, P, O, or N, with the proviso that one of Z, Z¹, Z², or Z³ is other than carbon, but is not O or S when attached to another Z atom by a double bond or when attached to another O or S atom. Furthermore, the optional substituents are understood to be attached to Z, Z¹, Z², or Z³ only when each is C. The term “heterocycle” also includes fully saturated ring structures, such as piperazinyl, dioxanyl, tetrahydrofuranyl, oxiranyl, aziridinyl, morpholinyl, pyrrolidinyl, piperidinyl, thiazolidinyl, and others. The term “heteroaryl” embraces unsaturated heterocyclic radicals. Examples of unsaturated heterocyclic radicals, also termed “heteroaryl” radicals include thienyl, pyrryl, furyl, pyridyl, pyrimidyl, pyrazinyl, pyrazolyl, oxazolyl, isoxazolyl, imidazolyl, thiazolyl, pyranyl, and tetrazolyl. The term also embraces radicals where heterocyclic radicals are fused with aryl radicals. Examples of such fused bicyclic radicals include benzofuran, benzothiophene, and the like. The terms aryl or heteroaryl, as appropriate, include the following structures:

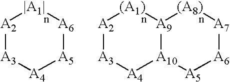

where:

when n=1, m=1 and A₁-A₈ are each CR^{x} or N, A₉ and A₁₀ are carbon;

when n=0, or 1, and m=0, or 1, one of A₂-A4 and/or A₅-A₇ is optionally S, O, or NR^{x}, and other ring members are CR^{x} or N, with the proviso that oxygen cannot be adjacent to sulfur in a ring. A₉ and A₁₀ are carbon;

when n is greater than or equal to 0, and m is greater than or equal to 0, 1 or more sets of 2 or more adjacent atoms A₁-A₁₀ are sp3 O, S, NR^{x}, CR^{x}R^{y}, or C═(O or S), with the proviso that oxygen and sulfur cannot be adjacent. The remaining A₁-A₈ are CR^{x} or N, and A₉ and A₁₀ are carbon;

when n is greater than or equal to 0, and m greater than or equal to 0, atoms separated by 2 atoms (i.e., A₁ and A4) are Sp3 O, S, NR^{x}, CR^{x}R^{y}, and remaining A₁-A₈ are independently CR^{x} or N, and A₉ and A₁₀ are carbon.

The term “sulfonyl”, whether used alone or linked to other terms such as alkylsulfonyl, denotes respectively divalent radicals —SO₂—. “Alkylsulfonyl”, embraces alkyl radicals attached to a sulfonyl radical, where alkyl is defined as above. The term “arylsulfonyl” embraces sulfonyl radicals substituted with an aryl radical. The terms “sulfamyl” or “sulfonamidyl”, whether alone or used with terms such as “N-alkylsulfamyl”, “N-arylsulfamyl”, “N,N-dialkylsulfamyl” and “N-alkyl-N-arylsulfamyl”, denotes a sulfonyl radical substituted with an amine radical, forming a sulfonamide (—SO₂—NH₂), which may also be termed an “aminosulfonyl”. The terms “N-alkylsulfamyl” and “N,N-dialkylsulfamyl” denote sulfamyl radicals substituted, respectively, with one alkyl radical, a cycloalkyl ring, or two alkyl radicals. The terms “N-arylsulfamyl” and “N-alkyl-N-arylsulfamyl” denote sulfamyl radicals substituted, respectively, with one aryl radical, and one alkyl and one aryl radical. The terms “carboxy” or “carboxyl”, whether used alone or with other terms, such as “carboxyalkyl”, denotes —CO₂—H. The term “carboxyalkyl” embraces radicals having a carboxyradical as defined above, attached to an alkyl radical. The term “carbonyl”, whether used alone or with other terms, such as “alkylcarbonyl”, denotes —(C═O)—. The term “alkylcarbonyl” embraces radicals having a carbonyl radical substituted with an alkyl radical. An example of an “alkylcarbonyl” radical is CH₃— (CO)—. The term “alkylcarbonylalkyl” denotes an alkyl radical substituted with an “alkylcarbonyl” radical. The term “alkoxycarbonyl” means a radical containing an alkoxy radical, as defined above, attached via an oxygen atom to a carbonyl (C═O) radical. Examples of such “alkoxycarbonyl” radicals include (CH₃)₃—C—O—C═O)— and —(O═)C—OCH₃. The term “alkoxycarbonylalkyl” embraces radicals having “alkoxycarbonyl”, as defined above substituted to an alkyl radical. Examples of such “alkoxycarbonylalkyl” radicals include (CH₃)₃C—OC(═O)—(CH₂)₂— and (CH₂)₂ (—O)COCH₃. The terms “amido”, or “carbamyl”, when used alone or with other terms such as “amidoalkyl”, “N-monoalkylamido”, “N-monoarylamido”, “N,N-dialkylamido”, “N-alkyl-N-arylamido”, “N-alkyl-N-hydroxyamido” and “N-alkyl-N-hydroxyamidoalkyl”, embraces a carbonyl radical substituted with an amino radical. The terms “N-alkylamido” and “N,N-dialkylamido” denote amido groups which have been substituted with one alkylradical and with two alkyl radicals, respectively. The terms “N-monoarylamido” and “N-alkyl-N-arylamido” denote amido radicals substituted, respectively, with one aryl radical, and one alkyl and one aryl radical. The term “N-alkyl-N-hydroxyamido” embraces amido radicals substituted with a hydroxyl radical and with an alkyl radical. The term “N-alkyl-N-hydroxyamidoalkyl” embraces alkylradicals substituted with an N-alkyl-N-hydroxyamido radical. The term “amidoalkyl” embraces alkyl radicals substituted with amido radicals. The term “aminoalkyl” embraces alkyl radicals substituted with amino radicals. The term “alkylaminoalkyl” embraces aminoalkyl radicals having the nitrogen atom substituted with an alkyl radical. The term “amidino” denotes an —C(—NH)—NH₂ radical. The term “cyanoamidin” denotes an —C(—N—CN)—NH₂ radical. The term “heterocycloalkyl” embraces heterocyclic-substituted alkyl radicals such as pyridylmethyl and thienylmethyl. The terms “aralkyl”, or “arylalkyl” embrace aryl-substituted alkyl radicals such as benzyl, diphenylmethyl, triphenylmethyl, phenethyl, and diphenethyl. The terms benzyl and phenylmethyl are interchangeable. The term “cycloalkyl” embraces radicals having three to ten carbon atoms, such as cyclopropyl cyclobutyl, cyclopentyl, cyclohexyl, and cycloheptyl. The term “cycloalkenyl” embraces unsaturated radicals having three to ten carbon atoms, such as cylopropenyl, cyclobutenyl, cyclopentenyl, cyclohexenyl, and cycloheptenyl. The term “alkylthio” embraces radicals containing a linear or branched alkyl radical, of one to ten carbon atoms, attached to a divalent sulfur atom. An example of “alkylthio” is methylthio, (CH₃—S—). The term “alkylsulfinyl” embraces radicals containing a linear or branched alkyl radical, of one to ten carbon atoms, attached to a divalent —S(—O)— atom. The terms “N-alkylamino” and “N,N-dialkylamino” denote amino groups which have been substituted with one alkyl radical and with two alkyl radicals, respectively. The term “acyl”, whether used alone, or within a term such as “acylamino”, denotes a radical provided by the residue after removal of hydroxyl from an organic acid. The term “acylamino” embraces an amino radical substituted with an acyl group. An examples of an “acylamino” radical is acetylamino (CH₃—C(═O)—NH—).

In the naming of substituent groups for general chemical structures, the naming of the chemical components of the group is typically from the terminal group-toward the parent compound unless otherwise noted, as discussed below. In other words, the outermost chemical structure is named first, followed by the next structure in line, followed by the next, etc. until the structure that is connected to the parent structure is named. For example, a substituent group having a structure such as:

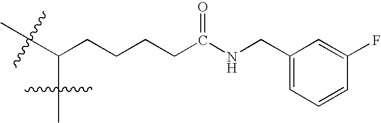

may be referred to generally as a “haloarylalkylaminocarboxylalkyl”. An example of one such group would be fluorophenylmethylcarbamylpentyl. The bonds having wavy lines through them represent the parent structure to which the alkyl is attached.

Substituent groups may also be named by reference to one or more “R” groups. The structure shown above would be included in a description, such as, “—C₁-C₆-alkyl-COR^{u}, where R^{u} is defined to include —NH—C₁-C₄-alkylaryl-R^{y}, and where R^{y} is defined to include halo. In this scheme, atoms having an “R” group are shown with the “R” group being the terminal group (i.e., furthest from the parent).

In another embodiment, the present cyclic pyrazole MK-2 inhibiting compound has the structure:

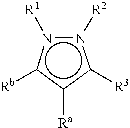

where:

R^{a} and R^{b} join to form a ring structure selected from the group consisting of:

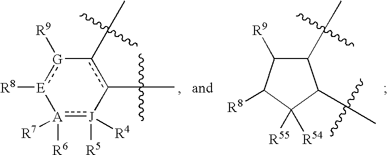

A, E, G and J are carbon, or optionally, one of A, E, G and J is selected from nitrogen, sulfur, or oxygen;

when A, E, G, or J is oxygen or sulfur, no substituent groups are bonded to the oxygen or the sulfur;

when A, E, G, or J is nitrogen, the nitrogen is optionally unsubstituted, or is substituted with R⁹;

each of R¹, R², R⁴ and R⁶ is optionally absent, or is independently selected from the R³ groups;

R³, R⁵, R⁷, R⁸, R⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴ and R⁵⁵ are each independently selected from —H, C₁-C₆ alkyl, C₂-C₆ alkenyl, C₂-C₆ alkynyl, nitro C₁-C₆ alkyl, azido C₁-C₆ alkyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁵, or R⁸ and R⁹ join to form a ring structure selected from:

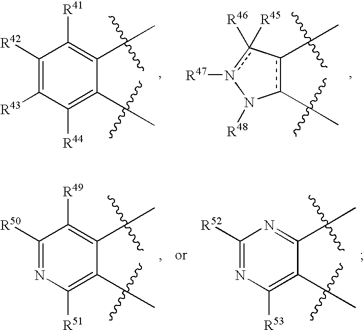

R¹⁰, R¹¹, are each independently selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, CO₂R¹⁹, COR²⁰, CONHR¹⁹, CON(R¹⁹)₂, SR¹⁸, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, arylaryl, arylheteroaryl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹², R¹³ are each independently selected from —H, C₁-C₆ alkyl, C₂-C₆ alkenyl, C₂-C₆ alkynyl, C₁-C₆ alkyl-NHR¹⁶, C₁-C₆ alkyl-NR¹⁶R¹⁷, C₁-C₄ alkyl-OR¹⁸, aryl, heteroaryl, heterocyclyl, CO₂R¹⁹, COR²⁰, CONHR¹⁹, CON(R¹⁹)₂, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁴ is selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, SR¹⁸, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁵ is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁶ and R¹⁷ are each independently selected from —H, C₁-C₆ alkyl, C₂-C₆ alkenyl, C₂-C₆ alkynyl, amino, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁶, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R¹⁸, R¹⁹ are each independently selected from —H, C₁-C₆ alkyl, C₂-C₆ alkenyl, C₂-C₆ alkynyl, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁷, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²⁰ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, O—R²⁴, C₁-C₄ alkyl-OR²⁴, aryl, heteroaryl, heterocyclyl, SR²⁴, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, arylalkoxyalkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²¹ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, nitro, cyano, O—R²⁴, C₁-C₄ alkyl-OR²⁴, aryl, heteroaryl, heterocyclyl, COR²⁶, SR²⁴, SOR²⁶, SO₂R²⁶, C₁-C₆ alkyl-COR²⁶, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²² and R²³ are each independently selected from —H, C₁-C₆ alkyl, C₂-C₆ alkenyl, C₂-C₆ alkynyl, amino, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁴ and R²⁵ are each independently selected from —H, C₁-C₆ alkyl, C₂-C₆ alkenyl, C₂-C₆ alkynyl, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁶ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, O—R³⁰, C₁-C₄ alkyl-OR³⁰, aryl, heteroaryl, heterocyclyl, SR³⁰, C₁-C₆ alkyl-CO₂R³¹, C₁-C₆ alkyl-COR³², C₁-C₆ alkyl-CONHR³¹, C₁-C₆ alkyl-CON(R³²)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁷ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, nitro, cyano, O—R³⁰, C₁-C₄ alkyl-OR³⁰, aryl, heteroaryl, heterocyclyl, COR³², SR³⁰, SOR³², SO₂R³², C₁-C₆ alkyl-COR³², C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁸ and R²⁹ are each independently selected from —H, C₁-C₆ alkyl, C₂-C₆ alkenyl, C₂-C₆ alkynyl, amino, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁰ and R³¹ are each independently selected from —H, C₁-C₆ alkyl, C₂-C₆ alkenyl, C₂-C₆ alkynyl, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³² is selected from —H, C₁-C₆ alkyl, C₂-C₆ alkenyl, C₂-C₆ alkynyl, amino, NHR³⁴, NR³⁴R³⁵, C₁-C₆ alkyl-NHR³⁴, C₁-C₆ alkyl-NR³⁴R³⁵, O—R³⁶, C₁-C₄ alkyl-OR³⁶, aryl, heteroaryl, heterocyclyl, SR³⁶, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³³ is selected from —H, C₁-C₆ alkyl, C₂-C₆ alkenyl, C₂-C₆ alkynyl, amino, NHR³⁴, NR³⁴R³⁵, C₁-C₆ alkyl-NHR³¹, C₁-C₆ alkyl-NR³⁴R³⁵, nitro, cyano, O—R³⁶, C₁-C₄ alkyl-OR³⁶, aryl, heteroaryl, heterocyclyl, COR³⁸, SR³⁵, SOR³⁸, SO₂R³⁸, C₁-C₆ alkyl-COR³⁸, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁴, R³⁵, R³⁶ and R³⁷ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁸ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁹ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, arylalkenyl, heterocyclylalkenyl, arylheterocyclylalkenyl, heterocyclylarylalkenyl, or heteroarylalkyl; and

R² and R³ optionally join to form a saturated or unsaturated ring of 5, 6, 7, or 8 atoms, where the atoms in the ring are independently selected from CR⁴⁰, C(R⁴⁰)₂, C═O, N, NR⁴⁰, O, S, C═S, S═O, or SO₂.

In another embodiment, the present cyclic pyrazole MK-2 inhibiting compound has the structure:

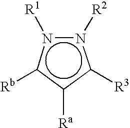

where:

R^{a} and R^{b} join to form a ring structure selected from the group consisting of:

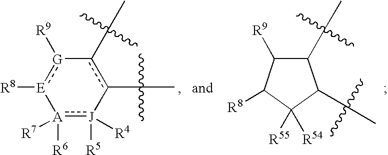

A, E, G and J are carbon, or optionally, one of A, E, G and J is selected from nitrogen, sulfur, or oxygen;

when A, E, G, or J is oxygen or sulfur, no substituent groups are bonded to the oxygen or the sulfur;

when A, E, G, or J is nitrogen, the nitrogen is optionally unsubstituted, or is substituted with R⁹;

R¹ is optionally absent, or is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, or C₇-C₁₀ mono- and bicyclic cycloalkyl, wherein mono- and bicyclic cycloalkyl are optionally substituted with one or more of the groups defined by R¹⁵;

each of R², R⁴ and R⁶ is optionally absent, or is independently selected from the R³ groups;

R³, R⁵, R⁷, R⁸, R⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴ and R⁵⁵ are each independently selected from —H, C₁-C₆ alkyl, C₂-C₆ alkenyl, C₂-C₆ alkynyl, nitro C₁-C₆ alkyl, azido C₁-C₆ alkyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁵; or R⁸ and R⁹ join to form a ring structure selected from:

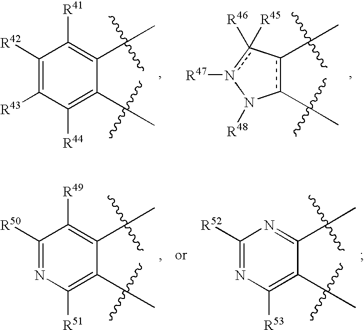

R¹⁰, R¹¹, are each independently selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, CO₂R¹⁹, COR²⁰, CONHR¹⁹, CON(R¹⁹)₂, SR¹⁸, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, arylaryl, arylheteroaryl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁰;

R¹², R¹³ are each independently selected from —H, C₁-C₆ alkyl, C₂-C₆ alkenyl, C₂-C₆ alkynyl, C₁-C₆ alkyl-NHR¹⁶, C₁-C₆ alkyl-NR¹⁶R¹⁷, C₁-C₄ alkyl-OR¹⁸, aryl, heteroaryl, heterocyclyl, CO₂R¹⁹, COR²⁰, CONHR¹⁹, CON(R¹⁹)₂, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁴ is selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, SR¹⁸, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁵ is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁶ and R¹⁷ are each independently selected from —H, C₁-C₆ alkyl, C₂-C₆ alkenyl, C₂-C₆ alkynyl, amino, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁶, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R¹⁸, R¹⁹ are each independently selected from —H, C₁-C₆ alkyl, C₂-C₆ alkenyl, C₂-C₆ alkynyl, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁷, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²⁰ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, O—R²⁴, C₁-C₄ alkyl-OR²⁴, aryl, heteroaryl, heterocyclyl, SR²⁴, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, arylalkoxyalkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²¹ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, nitro, cyano, O—R²⁴, C₁-C₄ alkyl-OR²⁴, aryl, heteroaryl, heterocyclyl, COR²⁶, SR²⁴, SOR²⁶, SO₂R²⁶, C₁-C₆ alkyl-COR²⁶, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²² and R²³ are each independently selected from —H, C₁-C₆ alkyl, C₂-C₆ alkenyl, C₂-C₆ alkynyl, amino, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁴ and R²⁵ are each independently selected from —H, C₁-C₆ alkyl, C₂-C₆ alkenyl, C₂-C₆ alkynyl, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁶ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, O—R³⁰, C₁-C₄ alkyl-OR³⁰, aryl, heteroaryl, heterocyclyl, SR³⁰, C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁷ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, nitro, cyano, O—R³⁰, C₁-C₄ alkyl-OR³⁰, aryl, heteroaryl, heterocyclyl, COR³², SR³⁰, SOR³², SO₂R³², C₁-C₆ alkyl-COR³², C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁸ and R²⁹ are each independently selected from —H, C₁-C₆ alkyl, C₂-C₆ alkenyl, C₂-C₆ alkynyl, amino, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁰ and R³¹ are each independently selected from —H, C₁-C₆ alkyl, C₂-C₆ alkenyl, C₂-C₆ alkynyl, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³² is selected from —H, C₁-C₆ alkyl, C₂-C₆ alkenyl, C₂-C₆ alkynyl, amino, NHR³⁴, NR³⁴R³⁵, C₁-C₆ alkyl-NHR³⁴, C₁-C₆ alkyl-NR³⁴R³⁵, O—R³⁶, C₁-C₄ alkyl-OR³⁶, aryl, heteroaryl, heterocyclyl, SR³⁶, C₁-C₆ alkyl-CC₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³³ is selected from —H, C₁-C₆ alkyl, C₂-C₆ alkenyl, C₂-C₆ alkynyl, amino, NHR³⁴, NR³⁴R³⁵, C₁-C₆ alkyl-NHR³¹, C₁-C₆ alkyl-NR³⁴R³⁵, nitro, cyano, O—R³⁶, C₁-C₄ alkyl-OR³⁶, aryl, heteroaryl, heterocyclyl, COR³⁸, SR³⁵, SOR³⁸, SO₂R³⁸, C₁-C₆ alkyl-COR³⁸, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁴, R³⁵, R³⁶ and R³⁷ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁸ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁹ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, arylalkenyl, heterocyclylalkenyl, arylheterocyclylalkenyl, heterocyclylarylalkenyl, or heteroarylalkyl; and

R² and R³ optionally join to form a saturated or unsaturated ring of 5, 6, 7, or 8 atoms, where the atoms in the ring are independently selected from CR⁴⁰, C(R⁴⁰)₂, C═O, N, NR⁴⁰, O, S, C═S, S═O, or SO₂.

In another embodiment, the present cyclic pyrazole MK-2 inhibiting compound has the structure:

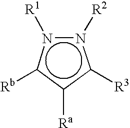

where:

R^{a} and R^{b} join to form a ring structure selected from the group consisting of:

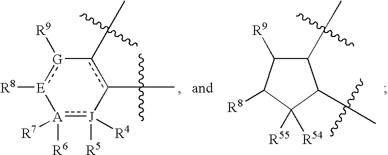

A, E, G and J are carbon, or optionally, one of A, E, G and J is selected from nitrogen, sulfur, or oxygen;

when A, E, G, or J is oxygen or sulfur, no substituent groups are bonded to the oxygen or the sulfur;

when A, E, G, or J is nitrogen, the nitrogen is optionally unsubstituted, or is substituted with R⁹;

each of R¹, R², R⁴ and R⁶ is optionally absent, or is independently selected from the R³ groups;

R³, R⁵, R⁷, R⁴⁰, R⁴¹, R⁴², R⁴³, R⁴⁴, R⁵⁴ and R⁵⁵ are each independently selected from —H, C₁-C₆ alkyl, C₂-C₆ alkenyl, C₂-C₆ alkynyl, nitro C₁-C₆ alkyl, azido C₁-C₆ alkyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁵;

R⁸ and R⁹ join to form the ring structure:

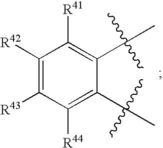

R¹⁰, R¹¹, are each independently selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, CO₂R¹⁹, COR²⁰, CONHR¹⁹, CON(R¹⁹)₂, SR¹⁸, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, arylaryl, arylheteroaryl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹², R¹³ are each independently selected from —H, C₁-C₆ alkyl, C₂-C₆ alkenyl, C₂-C₆ alkynyl, C₁-C₆ alkyl-NHR¹⁶, C₁-C₆ alkyl-NR¹⁶R¹⁷, C₁-C₄ alkyl-OR¹⁸, aryl, heteroaryl, heterocyclyl, CO₂R¹⁹, COR²⁰, CONHR¹⁹, CON(R¹⁹)₂, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁴ is selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, SR¹⁸, C₁-C₆ alkyl-CC₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁵ is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁶ and R¹⁷ are each independently selected from —H, C₁-C₆ alkyl, C₂-C₆ alkenyl, C₂-C₆ alkynyl, amino, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁶, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R¹⁸, R¹⁹ are each independently selected from —H, C₁-C₆ alkyl, C₂-C₆ alkenyl, C₂-C₆ alkynyl, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁷, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²⁰ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, O—R²⁴, C₁-C₄ alkyl-OR²⁴, aryl, heteroaryl, heterocyclyl, SR²⁴, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, arylalkoxyalkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²¹ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, nitro, cyano, O—R²⁴, C₁-C₄ alkyl-OR²⁴, aryl, heteroaryl, heterocyclyl, COR²⁶, SR²⁴, SOR²⁶, SO₂R²⁶, C₁-C₆ alkyl-COR²⁴, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²² and R²³ are each independently selected from —H, C₁-C₆ alkyl, C₂-C₆ alkenyl, C₂-C₆ alkynyl, amino, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁴ and R²⁵ are each independently selected from —H, C₁-C₆ alkyl, C₂-C₆ alkenyl, C₂-C₆ alkynyl, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁶ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, O—R³⁰, C₁-C₄ alkyl-OR³⁰, aryl, heteroaryl, heterocyclyl, SR³⁰, C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁷ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, nitro, cyano, O—R³⁰, C₁-C₄ alkyl-OR³⁰, aryl, heteroaryl, heterocyclyl, COR³², SR³⁰, SOR³², SO₂R³², C₁-C₆ alkyl-COR³², C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁸ and R²⁹ are each independently selected from —H, C₁-C₆ alkyl, C₂-C₆ alkenyl, C₂-C₆ alkynyl, amino, C₁-C₆ alkyl-NHR³⁴, C₁-C₆ alkyl-NR³⁴R³⁵, C₁-C₄ alkyl-OR³⁵, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁰ and R³¹ are each independently selected from —H, C₁-C₆ alkyl, C₂-C₆ alkenyl, C₂-C₆ alkynyl, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³² is selected from —H, C₁-C₆ alkyl, C₂-C₆ alkenyl, C₂-C₆ alkynyl, amino, NHR³⁴, NR³⁴R³⁵, C₁-C₆ alkyl-NHR³⁴, C₁-C₆ alkyl-NR³⁴R³⁵, O—R³⁶, C₁-C₄ alkyl-OR³⁶, aryl, heteroaryl, heterocyclyl, SR³⁶, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³³ is selected from —H, C₁-C₆ alkyl, C₂-C₆ alkenyl, C₂-C₆ alkynyl, amino, NHR³⁴, NR³⁴R³⁵, C₁-C₆ alkyl-NHR³¹, C₁-C₆ alkyl-NR³⁴R³⁵, nitro, cyano, O—R³⁶, C₁-C₄ alkyl-OR³⁶, aryl, heteroaryl, heterocyclyl, COR³⁸, SR³⁵, SOR³⁸, SO₂R³⁸, C₁-C₆ alkyl-COR³⁸, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁴, R³⁵, R³⁶ and R³⁷ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁸ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁹ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, arylalkenyl, heterocyclylalkenyl, arylheterocyclylalkenyl, heterocyclylarylalkenyl, or heteroarylalkyl; and

R² and R³ optionally join to form a saturated or unsaturated ring of 5, 6, 7, or 8 atoms, where the atoms in the ring are independently selected from CR⁴⁰, C(R⁴⁰)₂, C═O, N, NR⁴⁰, O, S, C═S, S═O, or SO₂.

In another embodiment, the present cyclic pyrazole MK-2 inhibiting compound has the structure:

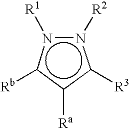

where:

R^{a} and R^{b} join to form a ring structure selected from the group consisting of:

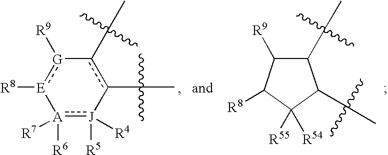

A, E, G and J are carbon, or optionally, one of A, E, G and J is selected from nitrogen, sulfur, or oxygen;

when A, E, G, or J is oxygen or sulfur, no substituent groups are bonded to the oxygen or the sulfur;

when A, E, G, or J is nitrogen, the nitrogen is optionally unsubstituted, or is substituted with R⁹;

each of R¹, R², R⁴ and R⁶ is optionally absent, or is independently selected from the R³ groups;

R³, R⁵, R⁷, R⁴⁰, R⁴⁵, R⁴⁶, R⁴⁷, R⁴⁸, R⁵⁴ and R⁵⁵ are each independently selected from —H, C₁-C₆ alkyl, C₂-C₆ alkenyl, C₂-C₆ alkynyl, nitro C₁-C₆ alkyl, azido C₁-C₆ alkyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁵;

R⁸ and R⁹ join to form the ring structure:

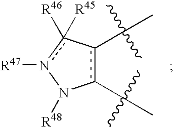

R¹⁰, R¹¹, are each independently selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, CO₂R¹⁹, COR²⁰, CONHR¹⁹, CON(R¹⁹)₂, SR¹⁹, SOR²⁰, SO₂R²⁰, C₁-C₆ alkyl-CO₂R¹⁹, C₁-C₆ alkyl-COR²⁷, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, arylaryl, arylheteroaryl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹², R¹³ are each independently selected from —H, C₁-C₆ alkyl, C₂-C₆ alkenyl, C₂-C₆ alkynyl, C₁-C₆ alkyl-NHR¹⁶, C₁-C₆ alkyl-NR¹⁶R¹⁷, C₁-C₄ alkyl-OR¹⁸, aryl, heteroaryl, heterocyclyl, CO₂R¹⁹, COR²⁰, CONHR¹⁹, CON(R¹⁹)₂, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁴ is selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, SR¹⁸, C₁-C₆ alkyl-CO₂R₁₉, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁵ is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹³, C₁-C₆ alkyl-SR¹⁴, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁶ and R¹⁷ are each independently selected from —H, C₁-C₆ alkyl, C₂-C₆ alkenyl, C₂-C₆ alkynyl, amino, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁶, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R¹⁸, R¹⁹ are each independently selected from —H, C₁-C₆ alkyl, C₂-C₆ alkenyl, C₂-C₆ alkynyl, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁷, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²⁰ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, O—R²⁴, C₁-C₄ alkyl-OR²⁴, aryl, heteroaryl, heterocyclyl, SR²⁴, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, arylalkoxyalkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²¹ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, nitro, cyano, O—R²⁴, C₁-C₄ alkyl-OR²⁴, aryl, heteroaryl, heterocyclyl, COR²⁶, SR²⁴, SOR²⁶, SO₂R²⁶, C₁-C₆ alkyl-COR²⁶, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²² and R²³ are each independently selected from —H, C₁-C₆ alkyl, C₂-C₆ alkenyl, C₂-C₆ alkynyl, amino, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁴ and R²⁵ are each independently selected from —H, C₁-C₆ alkyl, C₂-C₆ alkenyl, C₂-C₆ alkynyl, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁶ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, O—R³⁰, C₁-C₄ alkyl-OR³⁰, aryl, heteroaryl, heterocyclyl, SR³⁰, C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁷ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, nitro, cyano, O—R³⁰, C₁-C₄ alkyl-OR³⁰, aryl, heteroaryl, heterocyclyl, COR³², SR³⁰, SOR³², SO₂R³², C₁-C₆ alkyl-COR³², C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁸ and R²⁹ are each independently selected from —H, C₁-C₆ alkyl, C₂-C₆ alkenyl, C₂-C₆ alkynyl, amino, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁰ and R³¹ are each independently selected from —H, C₁-C₆ alkyl, C₂-C₆ alkenyl, C₂-C₆ alkynyl, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³² is selected from —H, C₁-C₆ alkyl, C₂-C₆ alkenyl, C₂-C₆ alkynyl, amino, NHR³⁴, NR³⁴R³⁵, C₁-C₆ alkyl-NHR³⁴, C₁-C₆ alkyl-NR³⁴R³⁵, O—R³⁶, C₁-C₄ alkyl-OR³⁶, aryl, heteroaryl, heterocyclyl, SR³⁶, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁵, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³³ is selected from —H, C₁-C₆ alkyl, C₂-C₆ alkenyl, C₂-C₆ alkynyl, amino, NHR³⁴, NR³⁴R³⁵, C₁-C₆ alkyl-NHR³¹, C₁-C₆ alkyl-NR³⁴R³⁵, nitro, cyano, O—R³⁶, C₁-C₄ alkyl-OR³⁶, aryl, heteroaryl, heterocyclyl, COR³⁸, SR³⁵, SOR³⁸, SO₂R³⁸, C₁-C₆ alkyl-COR³⁸, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁴, R³⁵, R³⁶ and R³⁷ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁸ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁹ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, arylalkenyl, heterocyclylalkenyl, arylheterocyclylalkenyl, heterocyclylarylalkenyl, or heteroarylalkyl; and

R² and R³ optionally join to form a saturated or unsaturated ring of 5, 6, 7, or 8 atoms, where the atoms in the ring are independently selected from CR⁴⁰, C(R⁴⁰)₂, C═O, N, NR⁴⁰, O, S, C═S, S═O, or SO₂.

Cyclic pyrazoles that are useful as the present MK-2 inhibiting compound have the structure:

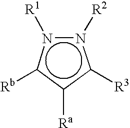

where:

R^{a} and R^{b} join to form a ring structure selected from the group consisting of:

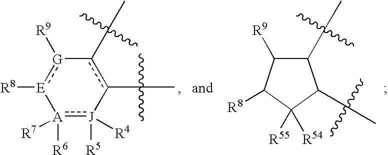

A, E, G and J are carbon, or optionally, one of A, E, G and J is selected from nitrogen, sulfur, or oxygen;

when A, E, G, or J is oxygen or sulfur, no substituent groups are bonded to the oxygen or the sulfur;

when A, E, G, or J is nitrogen, the nitrogen is optionally unsubstituted, or is substituted with R⁹;

each of R¹, R², R⁴ and R⁶ is optionally absent, or is independently selected from the R³ groups;

R³, R⁵, R⁷, R⁴⁰, R⁴⁹, R⁵⁰, R⁵¹, R⁵⁴ and R⁵⁵ are each independently selected from —H, C₁-C₆ alkyl, C₂-C₆ alkenyl, C₂-C₆ alkynyl, nitro C₁-C₆ alkyl, azido C₁-C₆ alkyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁵;

R⁸ and R⁹ join to form the ring structure:

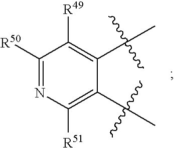

R¹⁰, R¹¹, are each independently selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, CO₂R¹⁹, COR²⁰, CONHR¹⁹, CON(R¹⁹)₂, SR¹⁸, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, arylaryl, arylheteroaryl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹², R¹³ are each independently selected from —H, C₁-C₆ alkyl, C₂-C₆ alkenyl, C₂-C₆ alkynyl, C₁-C₆ alkyl-NHR¹⁶, C₁-C₆ alkyl-NR¹⁶R¹⁷, C₁-C₄ alkyl-OR¹⁸, aryl, heteroaryl, heterocyclyl, CO₂R¹⁹, COR²⁰, CONHR¹⁹, CON(R¹⁹)₂, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁴ is selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, SR¹⁸, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR₁₃, C₁-C₆ alkyl-SOR²⁰, C₁-C₆ alkyl-SO₂R²⁰, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁰;

R¹⁵ is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁰;

R¹⁶ and R¹⁷ are each independently selected from —H, C₁-C₆ alkyl, C₂-C₆ alkenyl, C₂-C₆ alkynyl, amino, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁶, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R¹⁸, R¹⁹ are each independently selected from —H, C₁-C₆ alkyl, C₂-C₆ alkenyl, C₂-C₆ alkynyl, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁷, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²⁰ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, O—R²⁴, C₁-C₄ alkyl-OR²⁴, aryl, heteroaryl, heterocyclyl, SR²⁴, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, arylalkoxyalkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²¹ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, nitro, cyano, O—R²⁴, C₁-C₄ alkyl-OR²⁴, aryl, heteroaryl, heterocyclyl, COR²⁶, SR²⁴, SOR²⁶, SO₂R²⁶, C₁-C₆ alkyl-COR²⁶, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²² and R²³ are each independently selected from —H, C₁-C₆ alkyl, C₂-C₆ alkenyl, C₂-C₆ alkynyl, amino, C₁-C₆ alkyl-NHR²⁵, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁴ and R²⁵ are each independently selected from —H, C₁-C₆ alkyl, C₂-C₆ alkenyl, C₂-C₆ alkynyl, C₁-C₆ alkyl-NHR²⁸, C₁-C₆ alkyl-NR²⁸R²⁹., C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁶ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, O—R³⁰, C₁-C₄ alkyl-OR³⁰, aryl, heteroaryl, heterocyclyl, SR³⁰, C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁷ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, nitro, cyano, O—R³⁰, C₁-C₄ alkyl-OR³⁰, aryl, heteroaryl, heterocyclyl, COR³², SR³⁰, SOR³², SO₂R³², C₁-C₆ alkyl-COR³², C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁸ and R²⁹ are each independently selected from —H, C₁-C₆ alkyl, C₂-C₆ alkenyl, C₂-C₆ alkynyl, amino, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁰ and R³¹ are each independently selected from —H, C₁-C₆ alkyl, C₂-C₆ alkenyl, C₂-C₆ alkynyl, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³² is selected from —H, C₁-C₆ alkyl, C₂-C₆ alkenyl, C₂-C₆ alkynyl, amino, NHR³⁴, NR³⁴R³⁵, C₁-C₆ alkyl-NHR³⁴, C₁-C₆ alkyl-NR³⁴R³⁵, O—R³⁶, C₁-C₄ alkyl-OR³⁶, aryl, heteroaryl, heterocyclyl, SR³⁶, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³³ is selected from —H, C₁-C₆ alkyl, C₂-C₆ alkenyl, C₂-C₆ alkynyl, amino, NHR³⁴, NR³⁴R³⁵, C₁-C₆ alkyl-NHR³¹, C₁-C₆ alkyl-NR³⁴R³⁵, nitro, cyano, O—R³⁶, C₁-C₄ alkyl-OR³⁶, aryl, heteroaryl, heterocyclyl, COR³⁸, SR³⁵, SOR³⁸, SO₂R³⁸, C₁-C₆ alkyl-COR³⁸, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁴, R³⁵, R³⁶ and R³⁷ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁸ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁹ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, arylalkenyl, heterocyclylalkenyl, arylheterocyclylalkenyl, heterocyclylarylalkenyl, or heteroarylalkyl; and

R² and R³ optionally join to form a saturated or unsaturated ring of 5, 6, 7, or 8 atoms, where the atoms in the ring are independently selected from CR⁴⁰, C(R⁴⁰)₂, C═O, N, NR⁴⁰, O, S, C═S, S═O, or SO₂.

Cyclic pyrazoles that are useful as the present MK-2 inhibiting compound have the structure:

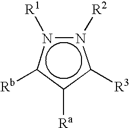

where:

R^{a} and R^{b} join to form a ring structure selected from the group consisting of:

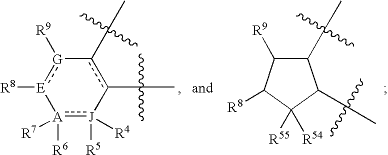

A, E, G and J are carbon, or optionally, one of A, E, G and J is selected from nitrogen, sulfur, or oxygen;

when A, E, G, or J is oxygen or sulfur, no substituent groups are bonded to the oxygen or the sulfur;

when A, E, G, or J is nitrogen, the nitrogen is optionally unsubstituted, or is substituted with R⁹;

each of R¹, R², R⁴ and R⁶ is optionally absent, or is independently selected from the R³ groups;

R³, R⁵, R⁷, R⁴⁰, R⁵², R⁵³, R⁵⁴ and R⁵⁵ are each independently selected from —H, C₁-C₆ alkyl, C₂-C₆ alkenyl, C₂-C₆ alkynyl, nitro C₁-C₆ alkyl, azido C₁-C₆ alkyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁵;

R⁸ and R⁹ join to form a ring structure selected from:

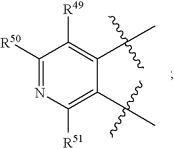

R¹⁰, R¹¹, are each independently selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, CO₂R¹⁹, COR²⁰, CONHR¹⁹, CON(R¹⁹)₂, SR¹⁸, SOR²⁰, SO₂R²⁰, C₁-C₆ alkyl-CO₂R⁰, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C alkyl-SOR²⁰, C₁-C₆ alkyl-SO₂R²⁰, halo, halo C₁-C₆ alkyl, arylaryl, arylheteroaryl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹², R¹³ are each independently selected from —H, C₁-C₆ alkyl, C₂-C₆ alkenyl, C₂-C₆ alkynyl, C₁-C₆ alkyl-NHR¹⁶, C₁-C₆ alkyl-NR¹⁶R¹⁷, C₁-C₄ alkyl-OR¹⁸, aryl, heteroaryl, heterocyclyl, CO₂R¹⁹, COR²⁰, CONHR¹⁹, CON(R¹⁹)₂, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁴ is selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, SR¹⁸, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁵ is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;

R¹⁶ and R¹⁷ are each independently selected from —H, C₁-C₆ alkyl, C₂-C₆ alkenyl, C₂-C₆ alkynyl, amino, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁶, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R¹⁸, R¹⁹ are each independently selected from —H, C₁-C₆ alkyl, C₂-C₆ alkenyl, C₂-C₆ alkynyl, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁷, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²⁰ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, O—R²⁴, C₁-C₄ alkyl-OR²⁴, aryl, heteroaryl, heterocyclyl, SR²⁴, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, arylalkoxyalkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²¹ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, nitro, cyano, O—R²⁴, C₁-C₄ alkyl-OR²⁴, aryl, heteroaryl, heterocyclyl, COR²⁶, SR²⁴, SOR²⁶, SO₂R²⁶, C₁-C₆ alkyl-COR²⁶, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;

R²² and R²³ are each independently selected from —H, C₁-C₆ alkyl, C₂-C₆ alkenyl, C₂-C₆ alkynyl, amino, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁴ and R²⁵ are each independently selected from —H, C₁-C₆ alkyl, C₂-C₆ alkenyl, C₂-C₆ alkynyl, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁶ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, O—R³⁰, C₁-C₄ alkyl-OR³⁰, aryl, heteroaryl, heterocyclyl, SR³⁰, C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁷ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, nitro, cyano, O—R³⁰, C₁-C₄ alkyl-OR³⁰, aryl, heteroaryl, heterocyclyl, COR³², SR³⁰, SOR³², SO₂R³², C₁-C₆ alkyl-COR³², C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;

R²⁸ and R²⁹ are each independently selected from —H, C₁-C₆ alkyl, C₂-C₆ alkenyl, C₂-C₆ alkynyl, amino, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁰ and R³¹ are each independently selected from —H, C₁-C₆ alkyl, C₂-C₆ alkenyl, C₂-C₆ alkynyl, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³² is selected from —H, C₁-C₆ alkyl, C₂-C₆ alkenyl, C₂-C₆ alkynyl, amino, NHR³⁴, NR³⁴R³⁵, C₁-C₆ alkyl-NHR³⁴, C₁-C₆ alkyl-NR³⁴R³⁵, O—R³⁶, C₁-C₄ alkyl-OR³⁶, aryl, heteroaryl, heterocyclyl, SR³⁶, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³³ is selected from —H, C₁-C₆ alkyl, C₂-C₆ alkenyl, C₂-C₆ alkynyl, amino, NHR³⁴, NR³⁴R³⁵, C₁-C₆ alkyl-NHR³¹, C₁-C₆ alkyl-NR³⁴R³⁵, nitro, cyano, O—R³⁶, C₁-C₄ alkyl-OR³⁵, aryl, heteroaryl, heterocyclyl, COR³⁸, SR³⁵, SOR³⁸, SO₂R³⁸, C₁-C₆ alkyl-COR³⁸, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁴, R³⁵, R³⁶ and R³⁷ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁸ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;

R³⁹ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, arylalkenyl, heterocyclylalkenyl, arylheterocyclylalkenyl, heterocyclylarylalkenyl, or heteroarylalkyl; and

R² and R³ optionally join to form a saturated or unsaturated ring of 5, 6, 7, or 8 atoms, where the atoms in the ring are independently selected from CR⁴⁰, C(R⁴⁰)₂, C═O, N, NR⁴⁰, O, S, C═S, S═O, or SO₂.

Cyclic pyrazoles having IC_{50 MK-2} values of less than about 100 include compounds having the structure:

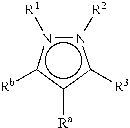

where:

R^{a} and R^{b} join to form the ring structure:

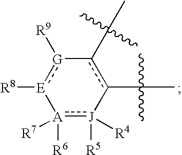

A, E, J and G are carbon;

R⁴, R⁵, and R⁶ are hydrogen;

R⁷ is selected from hydrogen or alkyl;

R⁸ and R⁹ join to form a ring structure that is selected from:

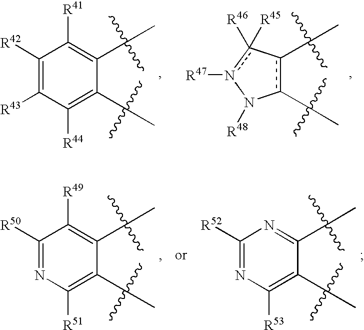

R¹ is optionally absent, or is selected from alkyl, alkenyl, haloalkyl, hydroxyalkyl, carbamylaryl, or arylalkoxycarbonylpiperidyl;

R² is optionally absent, or is selected from hydrogen, alkyl, alkenyl, aminoalkyl, alkoxyarylalkylaminoalkyl, alkylarylalkylaminoalkyl, haloalkylarylarylalkylaminoalkyl, nitroarylalkylaminoalkyl, aminoalkylarylarylalkylaminoalkyl, alkylsulfonylarylalkylaminoalkyl, alkylcarbonylarylalkylaminoalkyl, benzothienylarylalkylaminoalkyl, alkylfurylalkylaminoalkyl, cyanoarylalkylaminoalkyl, halothienylalkylaminoalkyl, haloarylalkylaminoalkyl, pyridylarylalkylaminoalkyl, haloarylalkylsulfonylaminoalkyl, arylalkylsulfonylaminoalkyl, arylarylalkylaminoalkyl, thienylalkylaminoalkyl, alkylarylarylalkylaminoalkyl, alkylarylarylalkylaminoalkyl, haloarylarylalkylaminoalkyl, haloalkylarylarylalkylaminoalkyl, furylarylalkylaminoalkyl, carboxylarylarylalkylaminoaryl, hydroxyalkyl, alkoxy(halo)arylalkylaminoalkyl, isoquinolinylaminoalkyl, hydroxyalkylcarbonylaminoalkyl, alkylaminoalkyl, alkoxycarbonylaminoalkyl, pyridylalkylaminoalkyl, aminoalkylarylaminoalkyl, alkenylaminoalkyl, aminoarylalkylaminoalkyl, alkoxyarylaminocarbonylaminoalkyl, cyclopropylalkylaminoalkyl, pyrroylalkylaminoalkyl, furylalkylaminoalkyl, piperidylalkyl, imidazolylalkylaminoalkyl, aminoarylaminoalkyl, pyridylcarbonylaminoalkyl, cyclopentylaminoalkyl, isoquinolinylalkylaminoalkyl, isoquinolinylamino(hydroxy)alkyl, pyrroylalkylaminoalkyl, arylalkylaminocarbonylaminoalkyl, cyanoalkyl, cyanoarylaminocarbonylaminoalkyl, haloalkyl, carboxy(amino)alkyl, aminocarbonylaminoalkyl, alkylsulfonylaminoalkyl, alkoxyarylalkylcarbonylaminoalkyl, thienylalkylamino(hydroxy)alkyl, cyano(amino)alkyl, pyridylaminoalkyl, alkoxycarbonylaminocarbonylaminoalkyl, carbamylpiperidylalkyl, carbamylalkyl, piperidyl, or arylalkylaminoalkyl;

R³ is selected from hydrogen, carboxyl, alkoxycarbonyl, carbamide, tetrazolyl, alkylthio, or R² and R³ optionally join to form a 6 or 7 membered heterocycle having 2 nitrogen atoms in the ring and with at least one ring nitrogen or carbon being substituted with a group selected from hydrogen, alkyl, oxo, haloalkyl, thienylalkylaminoalkyl, hydroxyalkyl, azidoalkyl, haloarylalkylaminoalkyl, nitroalkyl, carbamyl, alkoxycarbonyl, haloarylalkylsulfonylamino, or aminoalkyl;

R⁴¹, R⁴⁹ and R⁵³ are hydrogen;

R⁴² is selected from hydrogen, nitroaryl, haloalkyl, haloaryl, cyanoaryl, nitroaryl, alkylsulfonylaryl, aminoaryl, alkylamino, alkylaryl, pyridyl, carboxyaryl, arylalkyl, hydroxyl, amino, carboxyalkyl, alkyl, haloarylalkyl, aminosulfonyl, imidazolyl, indazolyl, pyridyl, indolyl, pyrazolyl, or halo;

R⁴³ is selected from hydroxy or alkoxy;

R⁴⁴ is selected from hydrogen or halo;

R⁴⁵ and R⁴⁷ are absent;

R⁴⁶ is selected from hydrogen, arylalkylthio, cyanoalkylthio, alkenylthio, imidazolyl, indazolyl, pyridyl, indolyl, pyrazolyl, or alkylthio;

R⁴⁸ is selected from hydrogen or arylalkyl;

R⁴⁹ and R⁵¹ are hydrogen;

R⁵⁰ is selected from hydrogen, halo, alkyl, benzodioxol, alkoxy, alkylaminoalkyl, isoquinolinyl, nitroaryl, alkoxyaryl, alkylaminoaryl, arylalkyl, hydroxyaryl, quinolinyl, hydroxyarylalkenyl, alkylaryl, haloaryl, haloalkylaryl, pyrazolyl, alkylsulfonylaryl, cyanoaryl, benzofuranyl, arylamino, haloarylalkyl, alkenyl, alkoxy, aminoaryl, haloarylalkenyl, alkoxyarylalkenyl, alkylthio, heterocyclyl, alkoxyarylalkylamino, arylalkylamino, hydroxypiperidyl, pyrrolidyl, tetrahydropyrazinoyl, arylalkoxyaryl, aminoalkylamino, cyano, piperizinyl, alkylpiperidinyl, alkylpiperizinyl, imidazolyl, indazolyl, pyridyl, indolyl, pyrazolyl, or naphthyl;

R⁵¹ is selected from hydrogen, aryl, halo, alkyl, arylamino, alkylthio, or alkoxy;

R⁵² is selected from hydrogen, quinolinyl, benzodioxyl, alkoxyaryl, morpholinyl, dihydroisoquinolinyl, imidazolyl, indazolyl, pyridyl, indolyl, pyrazolyl, or aryl; and

R⁵³ is selected from hydrogen, or halo.

Cyclic pyrazoles having IC_{50 MK-2} values of less than about 50 include compounds having the structure:

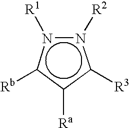

where:

R^{a} and R^{b} join to form the ring structure:

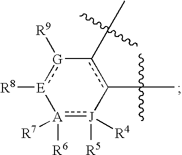

A, E, J and G are carbon;

R⁴, R⁵, and R⁶ are hydrogen;

R⁷ is selected from hydrogen or alkyl;

R⁸ and R⁹ join to form a ring structure that is selected from:

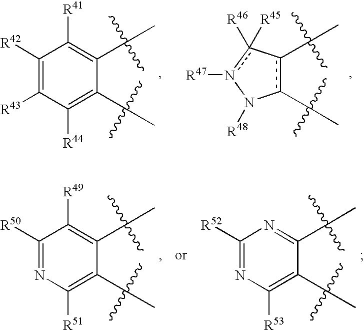

R¹ is absent, or is optionally arylalkoxycarbonylpiperidyl;

R² is selected from hydrogen, alkyl, alkenyl, aminoalkyl, alkoxyarylalkylaminoalkyl, alkylarylalkylaminoalkyl, haloalkylarylarylalkylaminoalkyl, nitroarylalkylaminoalkyl, aminoalkylarylarylalkylaminoalkyl, alkylsulfonylarylalkylaminoalkyl, alkylcarbonylarylalkylaminoalkyl, benzothienylarylalkylaminoalkyl, alkylfurylalkylaminoalkyl, cyanoarylalkylaminoalkyl, halothienylalkylaminoalkyl, haloarylalkylaminoalkyl, pyridylarylalkylaminoalkyl, haloarylalkylsulfonylaminoalkyl, arylalkylsulfonylaminoalkyl, arylarylalkylaminoalkyl, thienylalkylaminoalkyl, alkylarylarylalkylaminoalkyl, alkylarylarylalkylaminoalkyl, haloarylarylalkylaminoalkyl, haloalkylarylarylalkylaminoalkyl, furylarylalkylaminoalkyl, carboxylarylarylalkylaminoaryl, hydroxyalkyl, alkoxy(halo)arylalkylaminoalkyl, isoquinolinylaminoalkyl, hydroxyalkylcarbonylaminoalkyl, alkylaminoalkyl, alkoxycarbonylaminoalkyl, pyridylalkylaminoalkyl, aminoalkylarylaminoalkyl, alkenylaminoalkyl, aminoarylalkylaminoalkyl, alkoxyarylaminocarbonylaminoalkyl, cyclopropylalkylaminoalkyl, pyrroylalkylaminoalkyl, furylalkylaminoalkyl, piperidylalkyl, imidazolylalkylaminoalkyl, aminoarylaminoalkyl, pyridylcarbonylaminoalkyl, cyclopentylaminoalkyl, isoquinolinylalkylaminoalkyl, isoquinolinylamino(hydroxy)alkyl, pyrroylalkylaminoalkyl, arylalkylaminocarbonylaminoalkyl, cyanoalkyl, cyanoarylaminocarbonylaminoalkyl, haloalkyl, carboxy(amino)alkyl, aminocarbonylaminoalkyl, alkylsulfonylaminoalkyl, alkoxyarylalkylcarbonylaminoalkyl, thienylalkylamino(hydroxy)alkyl, cyano(amino)alkyl, pyridylaminoalkyl, alkoxycarbonylaminocarbonylaminoalkyl, carbamylpiperidylalkyl, carbamylalkyl, or arylalkylaminoalkyl;

R³ is selected from carboxyl, alkoxycarbonyl, carbamide, tetrazolyl, or R² and R³ optionally join to form a 6 or 7 membered heterocycle having 2 nitrogen atoms in the ring and with at least one ring nitrogen or carbon being substituted with a group selected from hydrogen, alkyl, oxo, haloalkyl, thienylalkylaminoalkyl, hydroxyalkyl, azidoalkyl, haloarylalkylaminoalkyl, nitroalkyl, carbamyl, alkoxycarbonyl, or aminoalkyl;

R⁴¹, R⁴⁹ and R⁵³ are hydrogen;

R⁴² is selected from hydrogen, haloalkyl, haloaryl, cyanoaryl, nitroaryl, alkylsulfonylaryl, aminoaryl, alkylamino, alkylaryl, carboxyaryl, arylalkyl, hydroxyl, amino, carboxyalkyl, alkyl, imidazolyl, indazolyl, pyridyl, indolyl, pyrazolyl, or halo;

R⁴³ is selected from hydroxy or alkoxy;

R⁴⁴ is selected from hydrogen or halo;

R⁴⁵ and R⁴⁷ are absent;

R⁴⁶ is selected from hydrogen, arylalkylthio, cyanoalkylthio, alkenylthio, imidazolyl, indazolyl, pyridyl, indolyl, pyrazolyl, or alkylthio;

R⁴⁸ is selected from hydrogen or arylalkyl;

R⁴⁹ and R⁵¹ are hydrogen;

R⁵⁰ is selected from hydrogen, halo, alkyl, benzodioxol, alkoxy, alkylaminoalkyl, isoquinolinyl, nitroaryl, alkoxyaryl, alkylaminoaryl, arylalkyl, hydroxyaryl, quinolinyl, hydroxyarylalkenyl, alkylaryl, haloaryl, haloalkylaryl, pyrazolyl, alkylsulfonylaryl, cyanoaryl, benzofuranyl, arylamino, haloalkylaryl, alkenyl, alkoxy, aminoaryl, haloarylalkenyl, alkoxyarylalkenyl, alkylthio, heterocyclyl, alkoxyarylalkylamino, arylalkylamino, hydroxypiperidyl, pyrrolidyl, tetrhydropyrazinoyl, arylalkoxyaryl, imidazolyl, indazolyl, pyridyl, indolyl, pyrazolyl, or naphthyl;

R⁵¹ is selected from hydrogen, halo, alkyl, or alkoxy;

R⁵² is selected from hydrogen, quinolinyl, benzodioxyl, alkoxyaryl, morpholinyl, dihydroisoquinolinyl, imidazolyl, indazolyl, pyridyl, indolyl, pyrazolyl, or aryl; and

R⁵³ is selected from hydrogen or halo.

Cyclic pyrazoles having IC_{50 MK-2} values of less than about 20 include compounds having the structure:

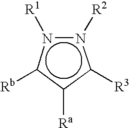

where:

R^{a} and R^{b} join to form the ring structure:

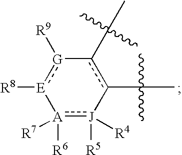

A, E, J and G are carbon;

R⁴, R⁵, and R⁶ are hydrogen;

R⁷ is selected from hydrogen or alkyl;

R⁸ and R⁹ join to form a ring structure that is selected from:

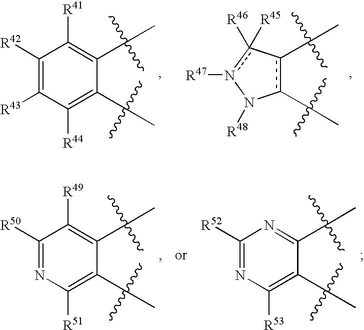

R¹ is absent;

R² is selected from alkyl, alkenyl, aminoalkyl, alkoxyarylalkylaminoalkyl, alkylarylalkylaminoalkyl, haloalkylarylarylalkylaminoalkyl, nitroarylalkylaminoalkyl, aminoalkylarylarylalkylaminoalkyl, alkylsulfonylarylalkylaminoalkyl, alkylcarbonylarylalkylaminoalkyl, benzothienylarylalkylaminoalkyl, alkylfurylalkylaminoalkyl, cyanoarylalkylaminoalkyl, halothienylalkylaminoalkyl, haloarylalkylaminoalkyl, pyridylarylalkylaminoalkyl, haloarylalkylsulfonylaminoalkyl, arylalkylsulfonylaminoalkyl, arylarylalkylaminoalkyl, thienylalkylaminoalkyl, alkylarylarylalkylaminoalkyl, alkylarylarylalkylaminoalkyl, haloarylarylalkylaminoalkyl, haloalkylarylarylalkylaminoalkyl, furylarylalkylaminoalkyl, carboxylarylarylalkylaminoaryl, hydroxyalkyl, alkoxy(halo)arylalkylaminoalkyl, isoquinolinylaminoalkyl, hydroxyalkylcarbonylaminoalkyl, hydrogen, alkylaminoalkyl, alkoxycarbonylaminoalkyl, pyridylalkylaminoalkyl, aminoalkylarylaminoalkyl, alkenylaminoalkyl, aminoarylalkylaminoalkyl, alkoxyarylaminocarbonylaminoalkyl, cyclopropylalkylaminoalkyl, pyrroylalkylaminoalkyl, furylalkylaminoalkyl, piperidylalkyl, imidazolylalkylaminoalkyl, aminoarylaminoalkyl, pyridylcarbonylaminoalkyl, cyclopentylaminoalkyl, isoquinolinylalkylaminoalkyl, isoquinolinylamino(hydroxy)alkyl, pyrroylalkylaminoalkyl, arylalkylaminocarbonylaminoalkyl, cyanoalkyl, or arylalkylaminoalkyl;

R³ is selected from carboxyl, alkoxycarbonyl, carbamide, tetrazolyl, or R² and R³ optionally join to form a 6 or 7 membered heterocycle having 2 nitrogen atoms in the ring and with at least one ring nitrogen or carbon being substituted with a group selected from hydrogen, alkyl, oxo, haloalkyl, thienylalkylaminoalkyl, hydroxyalkyl, azidoalkyl, haloarylalkylaminoalkyl, nitroalkyl, carbamyl, alkoxycarbonyl, or aminoalkyl;

R⁴¹, R⁴⁹ and R⁵³ are hydrogen;

R⁴² is selected from hydrogen, haloalkyl, haloaryl, cyanoaryl, nitroaryl, alkylsulfonylaryl, aminoaryl, alkylamino, alkylaryl, imidazolyl, indazolyl, pyridyl, indolyl, pyrazolyl, or halo;

R⁴³ is hydroxy;

R⁴⁴ is selected from hydrogen or halo;

R⁴⁵ and R⁴⁷ are absent;

R⁴⁶ is selected from hydrogen, arylalkylthio, cyanoalkylthio, alkenylthio, imidazolyl, indazolyl, pyridyl, indolyl, pyrazolyl, or alkylthio;

R⁴⁸, R⁴⁹ and R⁵¹ are hydrogen;

R⁵⁰ is selected from hydrogen, halo, benzodioxol, alkoxy, alkylaminoalkyl, isoquinolinyl, nitroaryl, alkoxyaryl, alkylaminoaryl, arylalkyl, hydroxyaryl, quinolinyl, hydroxyarylalkenyl, alkylaryl, haloaryl, haloalkylaryl, pyrazolyl, alkylsulfonylaryl, cyanoaryl, benzofuranyl, arylamino, haloalkylaryl, alkenyl, alkoxy, aminoaryl, haloarylalkenyl, alkoxyarylalkenyl, alkylthio, heterocyclyl, alkoxyarylalkylamino, arylalkylamino, hydroxypiperidyl, imidazolyl, indazolyl, pyridyl, indolyl, pyrazolyl, or naphthyl;

R⁵¹ is selected from hydrogen, or alkoxy;

R⁵² is selected from hydrogen, quinolinyl, benzodioxyl, alkoxyaryl, morpholinyl, dihydroisoquinolinyl, imidazolyl, indazolyl, pyridyl, indolyl, pyrazolyl, or aryl; and

R⁵³ is selected from hydrogen or halo.

Cyclic pyrazoles having IC_{50 MK-2} values of less than about 10 include compounds having the structure:

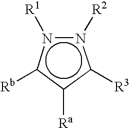

where:

R^{a} and R^{b} join to form the ring structure:

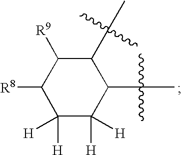

R⁸ and R⁹ join to form a ring structure that is selected from:

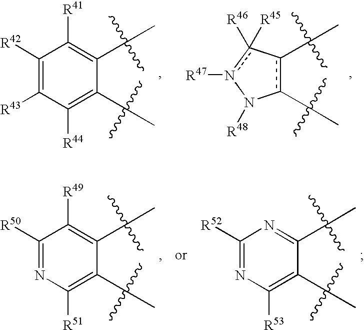

R¹ is absent;

R² is selected from alkyl, aminoalkyl, alkoxyarylalkylaminoalkyl, alkylarylalkylaminoalkyl, haloalkylarylarylalkylaminoalkyl, nitroarylalkylaminoalkyl, aminoalkylarylarylalkylaminoalkyl, alkylsulfonylarylalkylaminoalkyl, alkylcarbonylarylalkylaminoalkyl, benzothienylarylalkylaminoalkyl, alkylfurylalkylaminoalkyl, cyanoarylalkylaminoalkyl, halothienylalkylaminoalkyl, haloarylalkylaminoalkyl, pyridylarylalkylaminoalkyl, haloarylalkylsulfonylaminoalkyl, arylalkylsulfonylaminoalkyl, arylarylalkylaminoalkyl, thienylalkylaminoalkyl, alkylarylarylalkylaminoalkyl, alkylarylarylalkylaminoalkyl, haloarylarylalkylaminoalkyl, haloalkylarylarylalkylaminoalkyl, furylarylalkylaminoalkyl, carboxylarylarylalkylaminoaryl, hydroxyalkyl, alkoxy(halo)arylalkylaminoalkyl, isoquinolinylaminoalkyl, hydroxyalkylcarbonylaminoalkyl, hydrogen, alkylaminoalkyl, alkoxycarbonylaminoalkyl, pyridylalkylaminoalkyl, aminoalkylarylaminoalkyl, alkenylaminoalkyl, aminoarylalkylaminoalkyl, alkoxyarylaminocarbonylaminoalkyl, cyclopropylalkylaminoalkyl, pyrroylalkylaminoalkyl, furylalkylaminoalkyl, piperidylalkyl, imidazolylalkylaminoalkyl, aminoarylaminoalkyl, or arylalkylaminoalkyl;

R³ is selected from carboxyl, alkoxycarbonyl, carbamide, tetrazolyl, or R² and R³ optionally join to form a 6 or 7 membered heterocycle having 2 nitrogen atoms in the ring and with at least one ring nitrogen or carbon being substituted with a group selected from hydrogen, alkyl, oxo, haloalkyl, thienylalkylaminoalkyl, hydroxyalkyl, azidoalkyl, haloarylalkylaminoalkyl, nitroalkyl, carbamyl, or aminoalkyl;

R⁴¹, R⁴⁴, R⁴⁹ and R⁵³ are hydrogen;

R⁴² is selected from hydrogen, haloalkyl, haloaryl, cyanoaryl, nitroaryl, imidazolyl, indazolyl, pyridyl, indolyl, pyrazolyl, or halo;

R⁴³ is hydroxy;

R⁴⁵ and R⁴⁷ are absent;

R⁴⁶ is selected from hydrogen, arylalkylthio, cyanoalkylthio, imidazolyl, indazolyl, pyridyl, indolyl, pyrazolyl, or alkylthio;

R⁴⁸, R⁴⁹, R⁵¹ and R⁵³ are hydrogen;

R⁵⁰ is selected from hydrogen, halo, benzodioxol, alkoxy, alkylaminoalkyl, isoquinolinyl, nitroaryl, alkoxyaryl, alkylaminoaryl, arylalkyl, hydroxyaryl, quinolinyl, hydroxyarylalkenyl, alkylaryl, haloaryl, haloalkylaryl, pyrazolyl, alkylsulfonylaryl, cyanoaryl, benzofuranyl, arylamino, haloalkylaryl, alkenyl, alkoxy, aminoaryl, haloarylalkenyl, alkoxyarylalkenyl, alkylthio, heterocyclyl, alkoxyarylalkylamino, arylalkylamino, hydroxypiperidyl, imidazolyl, indazolyl, pyridyl, indolyl, pyrazolyl, or naphthyl;

R⁵¹ is selected from hydrogen, or alkoxy; and

R⁵² is selected from hydrogen, quinolinyl, benzodioxyl, alkoxyaryl, imidazolyl, indazolyl, pyridyl, indolyl, pyrazolyl, or aryl.

Cyclic pyrazoles having IC_{50 MK-2} values of less than about 1 include compounds having the structure:

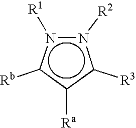

where:

R^{a} and R^{b} join to form the ring structure:

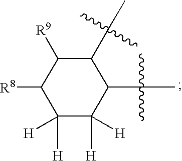

R⁸ and R⁹ join to form a ring structure that is selected from:

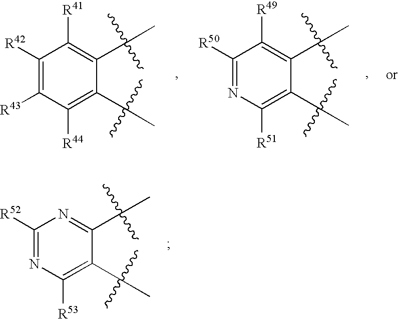

R¹ is absent;

R² is selected from alkyl, aminoalkyl, alkoxyarylalkylaminoalkyl, alkylarylalkylaminoalkyl, haloalkylarylarylalkylaminoalkyl, nitroarylalkylaminoalkyl, aminoalkylarylarylalkylaminoalkyl, alkylsulfonylarylalkylaminoalkyl, alkylcarbonylarylalkylaminoalkyl, benzothienylarylalkylaminoalkyl, alkylfurylalkylaminoalkyl, cyanoarylalkylaminoalkyl, halothienylalkylaminoalkyl, haloarylalkylaminoalkyl, pyridylarylalkylaminoalkyl, haloarylalkylsulfonylaminoalkyl, arylalkylsulfonylaminoalkyl, arylarylalkylaminoalkyl, thienylalkylaminoalkyl, alkylarylarylalkylaminoalkyl, alkylarylarylalkylaminoalkyl, haloarylarylalkylaminoalkyl, haloalkylarylarylalkylaminoalkyl, furylarylalkylaminoalkyl, carboxylarylarylalkylaminoaryl, or hydroxyalkyl;

R³ is selected from carboxyl, carbamide, or R² and R³ optionally join to form a 6 or 7 membered heterocycle having 2 nitrogen atoms in the ring and with at least one ring carbon being substituted with a group selected from hydrogen, oxo, thienylalkylaminoalkyl, hydroxyalkyl, or aminoalkyl;

R⁴¹, R⁴⁴, R⁴⁹ and R⁵³ are hydrogen;

R⁴² is selected from imidazolyl, indazolyl, pyridyl, indolyl, pyrazolyl, or nitroaryl;

R⁴³ is hydroxy;

R⁵⁰ is selected from hydrogen, halo, benzodioxol, alkoxy, alkylaminoalkyl, isoquinolinyl, nitroaryl, alkoxyaryl, alkylaminoaryl, arylalkyl, hydroxyaryl, quinolinyl, hydroxyarylalkenyl, alkylaryl, haloaryl, haloalkylaryl, pyrazolyl, alkylsulfonylaryl, cyanoaryl, imidazolyl, indazolyl, pyridyl, indolyl, pyrazolyl, or naphthyl;

R⁵⁰ is selected from hydrogen, or alkoxy; and

R⁵² is selected from hydrogen, quinolinyl, benzodioxyl, imidazolyl, indazolyl, pyridyl, indolyl, pyrazolyl, or aryl.

Examples of cyclic pyrazole MK-2 inhibitors of the present invention are shown in Tables 1-5. The tables include information about the chemical structure of the compound, and, if available, its chemical name and MK-2 and MK-3 inhibiting activity.

TABLE 1
Phenoxypyrazole MK-2 inhibitors

| | | | MK-2 | MK-3 |
|---|---|---|---|---|
| | | | Avg, IC50 | Avg. IC50 |
| No. | Structure^{a} | Chemical Name^{b} | (uM) | (uM) |
| 1 | 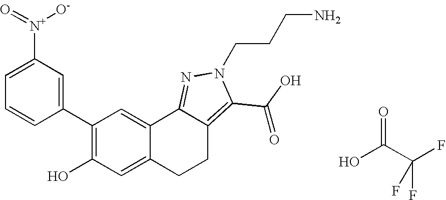 | 2-(3-aminopropyl)-7-hydroxy-8-(3- nitrophenyl)-4,5-dihydro-2H- benzo[g]indazole-3-carboxylic acid trifluoroacetate | 0.823 | 115 |
| 2 | 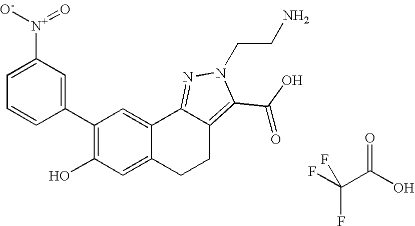 | 2-(2-aminoethyl)-7-hydroxy-8-(3- nitrophenyl)-4,5-dihydro-2H- benzo[g]indazole-3-carboxylic acid trifluoroacetate | 0.845 | 40.9 |
| 3 | 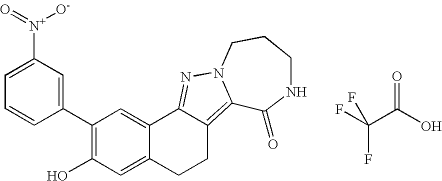 | 3-hydroxy-2-(3-nitrophenyl)- 5,6,8,9,10,11-hexahydro-7H- benzo[g][1,4]diazepino[1,2- b[indazole-7-one trifluoroacetate | 1.48 | >200 |
| 4 | 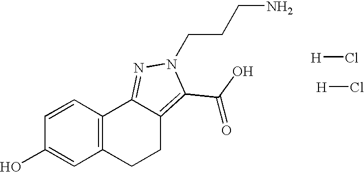 | 2-(3-aminopropyl)-7-hydroxy-4,5- dihydro-2H-benzo[g]indazole-3- carboxylic acid dihydrochloride | 2.09 | 37.9 |
| 5 | 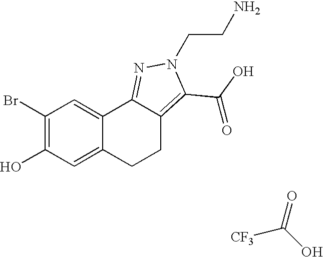 | 2-(2-aminoethyl)-8-bromo-7- hydroxy-4,5-dihydro-2H- benzo[g]indazole-3-carboxylic acid trifluoroacetate | 2.14 | >200 |
| 6 | 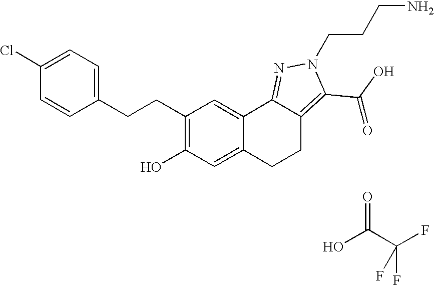 | 2-(3-aminopropyl)-8-[2-(4- chlorophenyl)ethyl]-7-hydroxy-4,5- dihydro-2H-benzo[g]indazole-3- carboxylic acid trifluoroacetate | 2.29 | 38.2 |
| 7 | 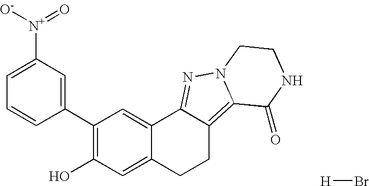 | 3-hydroxy-2-(3-nitrophenyl) 5,6,9,10- tetrahydrobenzo[g]pyrazino[1,2- b]indazol-7(8H)-one hydrobromide | 3 | >200 |
| 8 | 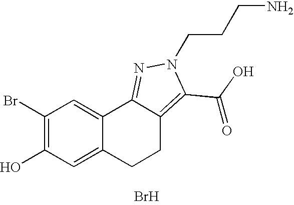 | 2-(3-aminopropyl)-8-bromo-7- hydroxy-4,5-dihydro-2H- benzo[g]indazole-3-carboxylic acid hydrobromide | 3.75 | >200 |
| 9 | 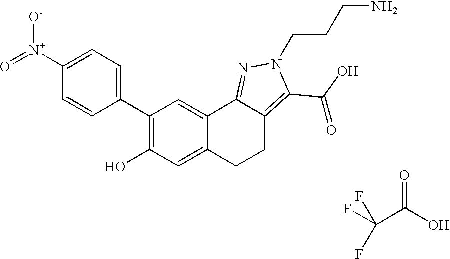 | 2-(3-aminopropyl)-7-hydroxy-8-(4- nitrophenyl)-4,5-dihydro-2H- benzo[g]indazole-3-carboxylic acid trifluoroacetate | 4.91 | 104 |
| 10 | 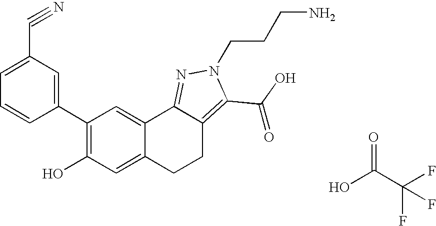 | 2-(3-aminopropyl)-8-(3- cyanophenyl)-7-hydroxy-4,5- dihydro-2H-benzo[g]indazole-3- carboxylic acid trifluoroacetate | 5.35 | 168 |
| 11 | 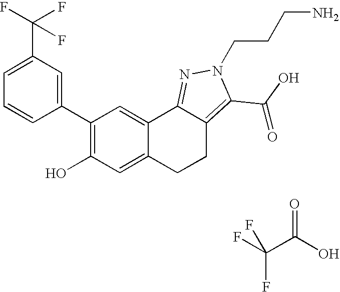 | 2-(3-aminopropyl)-7-hydroxy-8-[3- (trifluoromethyl)phenyl]-4,5-dihydro- 2H-benzo[g]indazole-3-carboxylic acid trifluoroacetate | 7.53 | 71.7 |
| 12 | 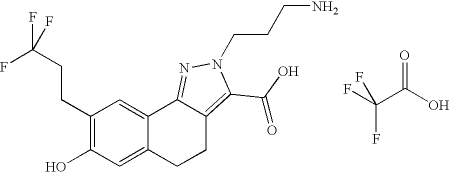 | 2-(3-aminopropyl)-7-hydroxy-8- (3,3,3-trifluoropropyl)-4,5-dihydro- 2H-benzo[g]indazole-3-carboxylic acid trifluoroacetate | 8.11 | 149 |
| 13 | 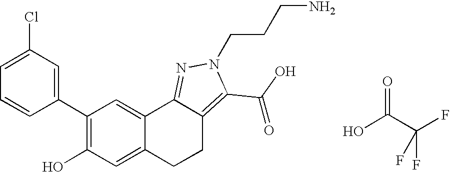 | 2-(3-aminopropyl)-8-(3- chlorophenyl)-7-hydroxy-4,5- dihydro-2H-benzo[g]indazole-3-carboxylic acid trifluoroacetate | 10.5 | 121 |
| 14 | 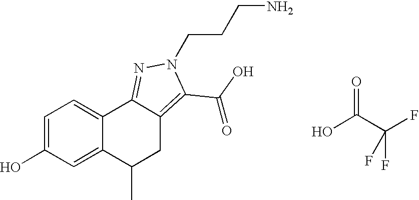 | 2-(3-aminopropyl)-7-hydroxy-5- methyl-4,5-dihydro-2H- benzo[g]indazole-3-carboxylic acid trifluoroacetate | 10.7 | >200 |
| 15 | 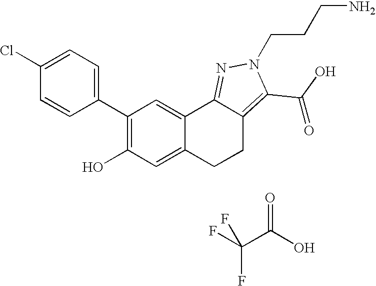 | 2-(3-aminopropyl)-8-(4- chlorophenyl)-7-hydroxy-4,5- dihydro-2H-benzo[g]indazole-3- carboxylic acid trifluoroacetate | 11 | 109 |
| 16 | 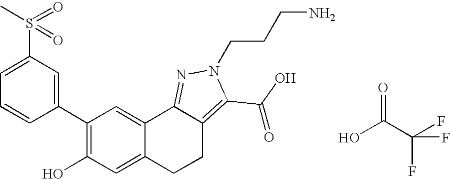 | 2-(3-aminopropyl)-7-hydroxy-8-[3- (methylsulfonyl)phenyl]-4,5-dihydro 2H-benzo[g]indazole-3-carboxylic acid trifluoroacetate | 12.9 | >200 |
| 17 | 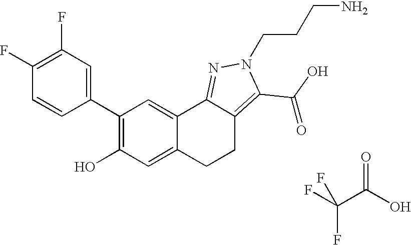 | 2-(3-aminopropyl)-8-(3,4- difluorophenyl)-7-hydroxy-4,5- dihydro-2H-benzo[g]indazole-3- carboxylic acid trifluoroacetate | 14.2 | 167 |
| 18 | 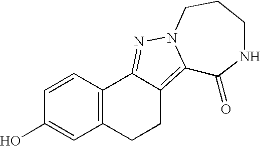 | 3-hydroxy-5,6,8,9,10,11-hexahydro 7H-benzo[g][1,4]diazepino[1,2- b]indazol-7-one | 17 | |
| 19 | 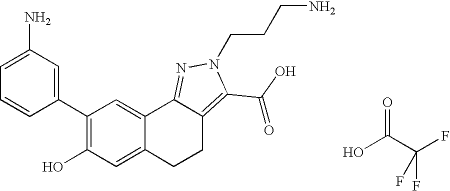 | 8-(3-aminophenyl)-2-(3- aminopropyl)-7-hydroxy-4,5- dihydro-2H-benzo[g]indazole-3- carboxylic acid trifluoroacetate | 17.2 | 166 |
| 20 | 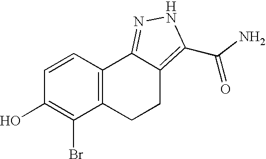 | 6-bromo-7-hydroxy-4,5-dihydro-2H- benzo[g]indazole-3-carboxamide | 17.6 | >200 |
| 21 | 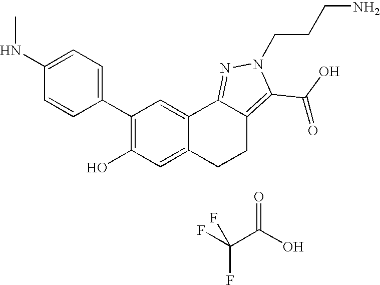 | 2-(3-aminopropyl)-7-hydroxy-8-[4- (methylamino)phenyl]-4,5-dihydro- 2H-benzo[g]indazole-3-carboxylic acid trifluoroacetate | 17.7 | >200 |
| 22 | 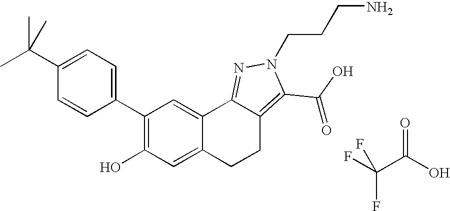 | 2-(3-aminopropyl)-8-(4-tert- butylphenyl)-7-hydroxy-4,5-dihydro- 2H-benzo[g]indazole-3-carboxylic acid trifluoroacetate | 18.8 | 120 |
| 23 | 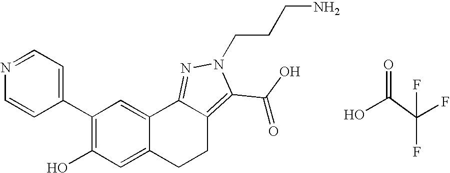 | 2-(3-aminopropyl)-7-hydroxy-8- pyridin-4-yl-4,5-dihydro-2H- benzo[g]indazole-3-carboxylic acid trifluoroacetate | 18.8 | >200 |
| 24 | 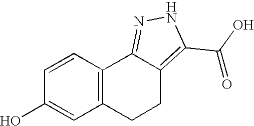 | 7-hydroxy-4,5-dihydro-2H- benzo[g]indazole-3-carboxylic acid | 19.6 | >200 |
| 25 | 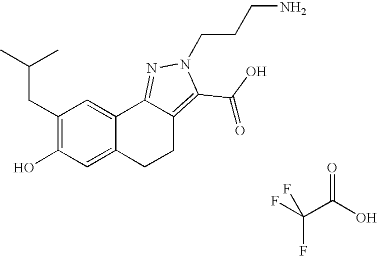 | 2-(3-aminopropyl)-7-hydroxy-8- isobutyl-4,5-dihydro-2H- benzo[g]indazole-3-carboxylic acid trifluoroacetate | 20.8 | |
| 26 | 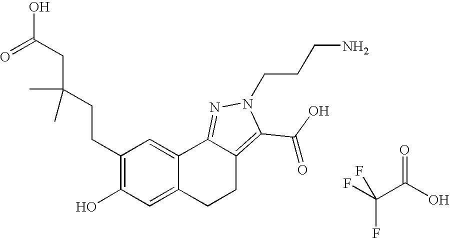 | 2-(3-aminopropyl)-8-(4-carboxy-3,3 dimethylbutyl)-7-hydroxy-4,5- dihydro-2H-benzo[g]indazole-3- carboxylic acid trifluoroacetate | 22.2 | 135 |
| 27 | 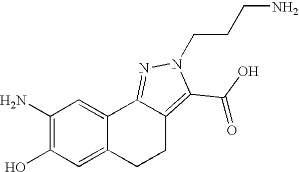 | 8-amino-2-(3-aminopropyl)-7- hydroxy-4,5-dihydro-2H- benzo[g]indazole-3-carboxylic acid | 26.8 | >200 |
| 28 | 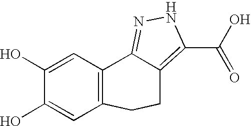 | 7,8-dihydroxy-4,5-dihydro-2H- benzo[g]indazole-3-carboxylic acid | 28.3 | >200 |
| 29 | 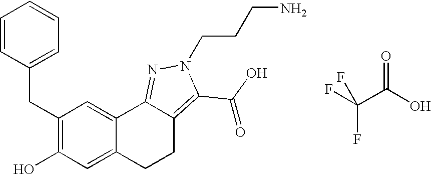 | 2-(3-aminopropyl)-8-benzyl-7- hydroxy-4,5-dihydro-2H- benzo[g]indazole-3-carboxyiic acid trifluoroacetate | 29.6 | |
| 30 | 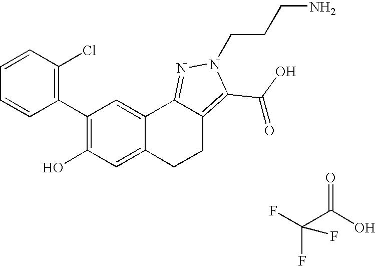 | 2-(3-aminopropyl)-8-(2- chlorophenyl)-7-hydroxy-4,5- dihydro-2H-benzo[g]indazole-3- carboxylic acid trifluoroacetate | 29.8 | 60.7 |
| 31 | 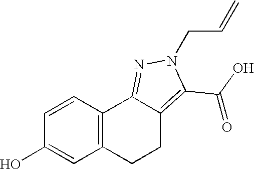 | 2-allyl-7-hydroxy-4,5-dihydro-2H- benzo[g]indazole-3-carboxylic acid | 35.6 | >200 |
| 32 | 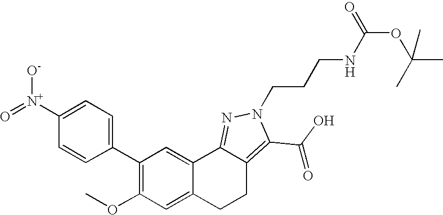 | 2-{3-[(tert- butoxycarbonyl)amino]propyl}-7- methoxy-8-(4-nitrophenyl)-4,5- dihydro-2H-benzo[g]indazole-3- carboxylic acid | 37.4 | 115 |
| 33 | 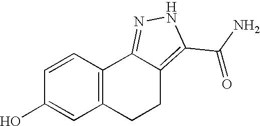 | | 39.2 | 107 |
| 34 | 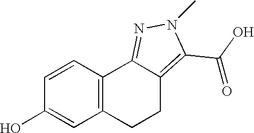 | 7-hydroxy-2-methyl-4,5-dihydro-2H benzo[g]indazole-3-carboxylic acid | 44.4 | >200 |
| 35 | 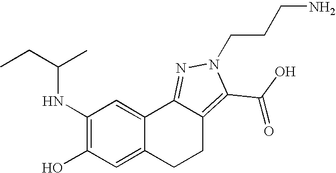 | 2-(3-aminopropyl)-8-(sec- butylamino)-7-hydroxy-4,5-dihydro- 2H-benzo[g]indazole-3-carboxylic acid | 45.9 | |
| 36 | 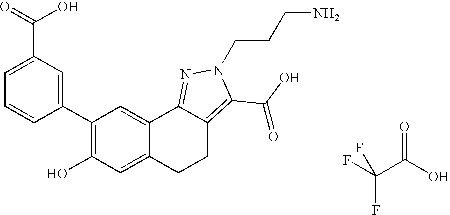 | 2-(3-aminopropyl)-8-(3- carboxyphenyl)-7-hydroxy-4,5- dihydro-2H-benzo[g]indazole-3- carboxylic acid trifluoroacetate | 47.3 | >200 |
| 37 | 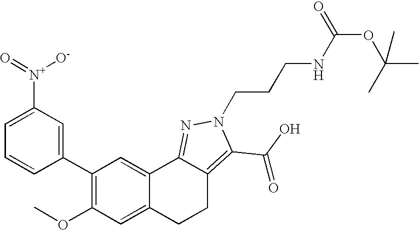 | 2-{3-[(tert- butoxycarbonyl)amino]propyl}-7- methoxy-8-(3-nitrophenyl)-4,5- dihydro-2H-benzo[g]indazole-3- carboxylic acid | 53.7 | >200 |
| 38 | 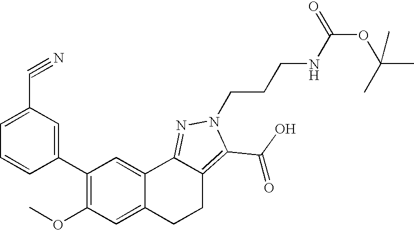 | 2-{3-[(tert- butoxycarbonyl)amino]propyl}-8-(3- cyanophenyl)-7-methoxy-4,5- dihydro-2H-benzo[g]indazole-3- carboxylic acid | 56.2 | >200 |
| 39 | 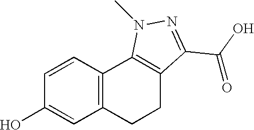 | 7-hydroxy-1-methyl-4,5-dihydro-1H benzo[g]indazole-3-carboxylic acid | 56.2 | >200 |
| 40 | 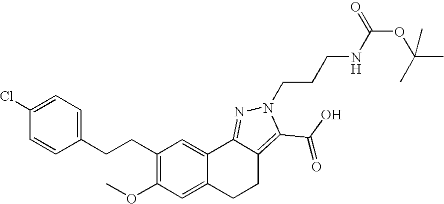 | 2-{3-[(tert- butoxycarbonyl)amino]propyl}-8-[2- (4-chlorophenyl)ethyl]-7-methoxy- 4,5-dihydro-2H-benzo[g]indazole-3- carboxylic acid | 59.6 | >200 |
| 41 | 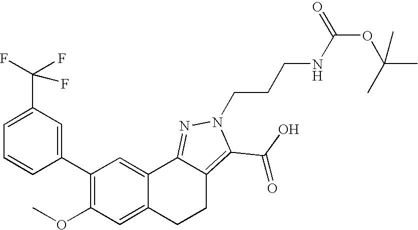 | 2-{3-[(tert- butoxycarbonyl)amino]propyl}-7- methoxy-8-[3- (trifluoromethyl)phenyl]-4,5-dihydro 2H-benzo[g]indazole-3-carboxylic acid | 63.8 | >200 |
| 42 | 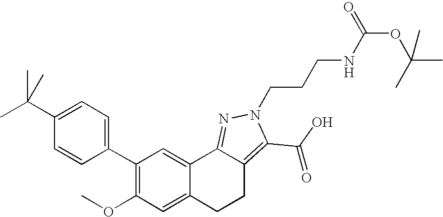 | 2-{3-[(tert- butoxycarbonyl)amino]propyl}-8-(4- tert-butylphenyl)-7-methoxy-4,5- dihydro-2H-benzo[g]indazole-3- carboxylic acid | 65.7 | >200 |
| 43 | 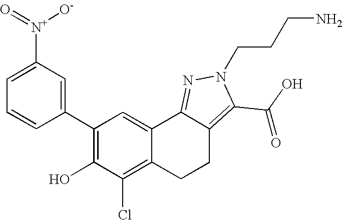 | 2-(3-aminopropyl)-6-chloro-7- hydroxy-8-(3-nitrophenyl)-4,5- dihydro-2H-benzo[g]indazole-3- carboxylic acid | 74.3 | |
| 44 | 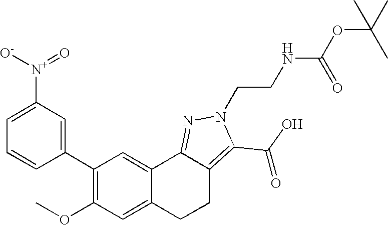 | 2-{2-[(tert- butoxycarbonyl)amino]ethyl}-7- methoxy-8-(3-nitrophenyl)-4,5- dihydro-2H-benzo[g]indazole-3- carboxylic acid | 82.2 | >200 |
| 45 | 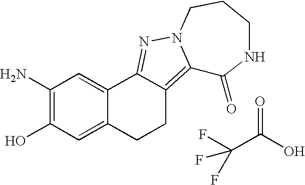 | 2-amino-3-hydroxy-5,6,8,9,10,11- hexahydro-7H- benzo[g][1,4]diazepino[1,2- b]indazol-7-one trifluoroacetate | 88.7 | >200 |
| 46 | 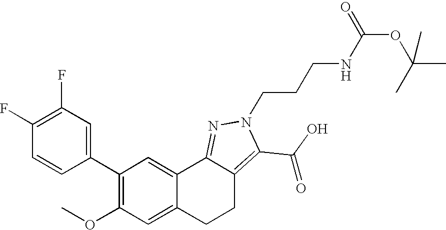 | 2-{3-[(tert- butoxycarbonyl)amino]propyl}-8- (3,4-difluorophenyl)-7-methoxy-4,5- dihydro-2H-benzo[g]indazole-3- carboxylic acid | 92.7 | >200 |
| 47 | 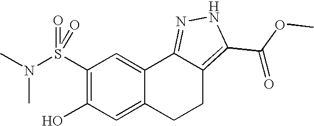 | methyl 8-[(dimethylamino)sulfonyl]- 7-hydroxy-4,5-dihydro-2H- benzo[g]indazole-3-carboxylate | 92.8 | |
| 48 | 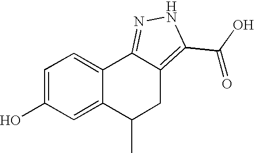 | 7-hydroxy-5-methyl-4,5-dihydro-2H benzo[g]indazole-3-carboxylic acid | 97.1 | >200 |
| 49 | 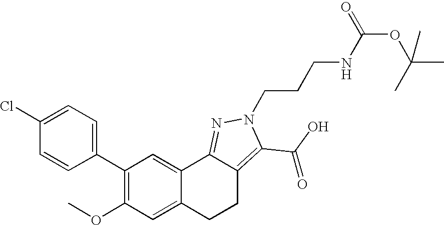 | 2-{3-[(tert- butoxycarbonyl)amino]propyl}-8-(4- chlorophenyl)-7-methoxy-4,5- dihydro-2H-benzo[g]indazole-3- carboxylic acid | 98.1 | 161 |
| 50 | 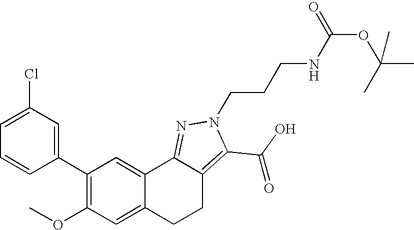 | 2-{3-[(tert- butoxycarbonyl)amino]propyl}-8-(3- chlorophenyl)-7-methoxy-4,5- dihydro-2H-benzo[g]indazole-3- carboxylic acid | 104 | |
| 51 | 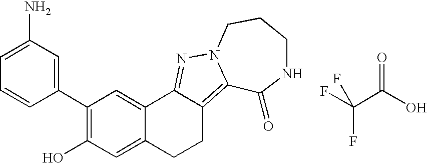 | 2-(3-aminophenyl)-3-hydroxy- 5,6,8,9,10,11-hexahydro-7H- benzo[g][1,4]diazepino[1,2- b]indazol-7-one trifluoroacetate | 105 | |
| 52 | 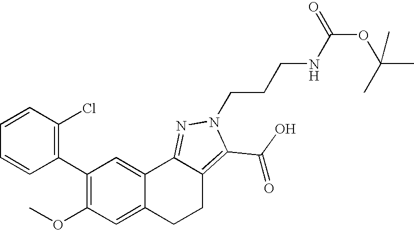 | 2-{3-[(tert- butoxycarbonyl)amino]propyl}-8-(2- chlorophenyl)-7-methoxy-4,5- dihydro-2H-benzo[g]indazole-3- carboxylic acid | 109 | >200 |
| 53 | 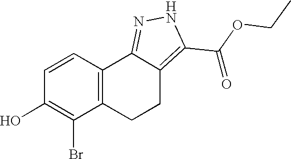 | ethyl 6-bromo-7-hydroxy-4,5- dihydro-2H-benzo[g]indazole-3- carboxylate | 116 | >200 |
| 54 | 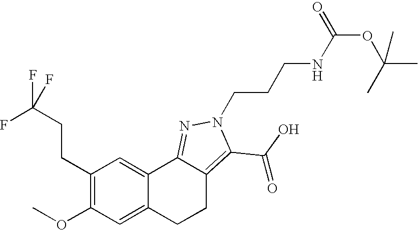 | 2-{3-[(tert- butoxycarbonyl)amino]propyl}-7- methoxy-8-(3,3,3-trifluoropropyl)- 4,5-dihydro-2H-benzo[g]indazole-3- carboxyiic acid | 134 | >200 |
| 55 | 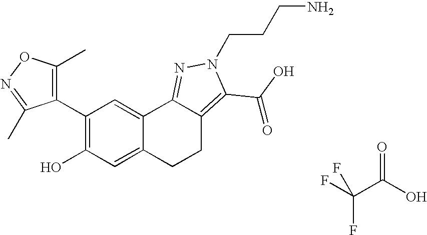 | 2-(3-aminopropyl)-8-(3,5- dimethylisoxazol-4-yl)-7-hydroxy- 4,5-dihydro-2H-benzo[g]indazole-3- carboxylic acid trifluoroacetate | 136 | 169 |
| 56 | 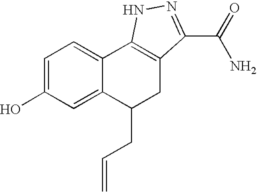 | 5-allyl-7-hydroxy-4,5-dihydro-1H- benzo[g]indazole-3-carboxamide | 139 | >200 |
| 57 | 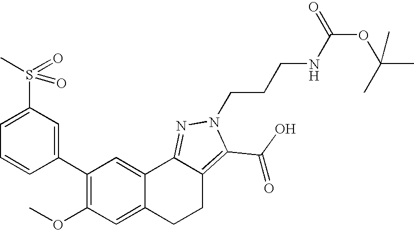 | 2-{3-[(tert- butoxycarbonyl)amino]propyl}-7- methoxy-8-[3- (methylsulfonyl)phenyl]-4,5-dihydro 2H-benzo[g]indazole-3-carboxylic acid | 143 | >200 |
| 58 | 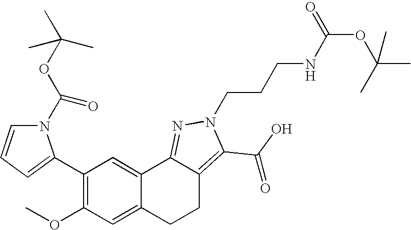 | 2-{3-[(tert- butoxycarbonyl)amino]propyl}-8-[1- N(tert-butoxycarbonyl)-1H-pyrrol-2- yl]-7-methoxy-4,5-dihydro-2H- benzo[g]indazole-3-carboxylic acid | 148 | >200 |
| 59 | 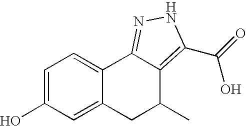 | 7-hydroxy-4-methyl-4,5-dihydro-2H benzo[g]indazole-3-carboxylic acid | 148 | >200 |
| 60 | 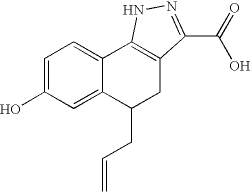 | 5-allyl-7-hydroxy-4,5-dihydro-1H- benzo[g]indazole-3-carboxylic acid | 148 | >200 |
| 61 | 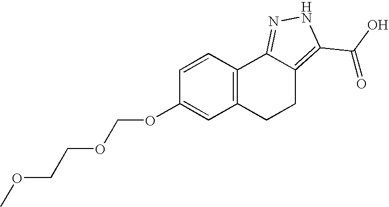 | 7-[(2-methoxyethoxy)methoxy]-4,5- dihydro-2H-benzo[g]indazole-3- carboxylic acid | 150 | >200 |
| 62 | 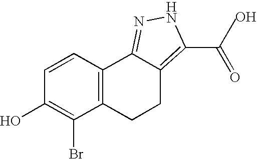 | 6-bromo-7-hydroxy-4,5-dihydro-2H- benzo[g]indazole-3-carboxylic acid | 152 | >200 |
| 63 | 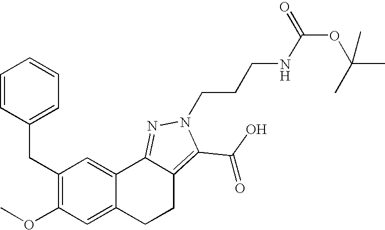 | 8-benzyl-2-{3-[(tert- butoxycarbonyl)amino]propyl}-7- methoxy-4,5-dihydro-2H- benzo[g]indazole-3-carboxylic acid | 154 | |
| 64 | 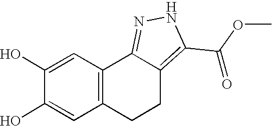 | methyl 7,8-dihydroxy-4,5-dihydro- 2H-benzo[g]indazole-3-carboxylate | 154 | >200 |
| 65 | 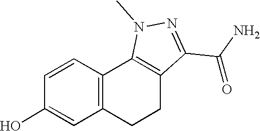 | 7-hydroxy-1-methyl-4,5-dihydro-1H benzo[g]indazole-3-carboxamide | 158 | >200 |
| 66 | 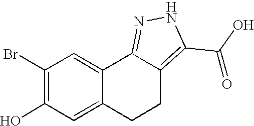 | 8-bromo-7-hydroxy-4,5-dihydro-2H- benzo[g]indazole-3-carboxylic acid | 163 | |
| 67 | 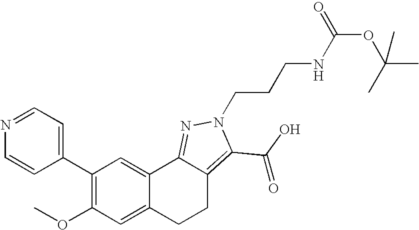 | 2-{3-[(tert- butoxycarbonyl)amino]propyl}-7- methoxy-8-pyridin-4-yl-45-dihydro- 2H-benzo[g]indazole-3-carboxylic acid | 165 | >200 |
| 68 | 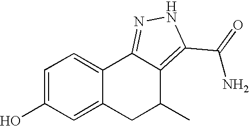 | 7-hydroxy-4-methyl-4,5-dihydro-2H benzo[g]indazole-3-carboxamide | 173 | 196 |
| 69 | 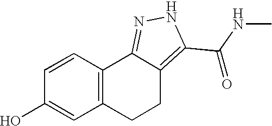 | 7-hydroxy-N-methyl-4,5-dihydro-2H benzo[g]indazole-3-carboxamide | 176 | >200 |
| 70 | 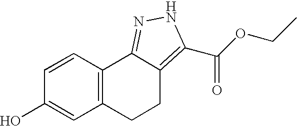 | | 176 | >200 |
| 71 | 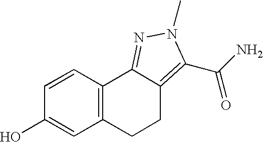 | 7-hydroxy-2-methyl-4,5-dihydro-2H benzo[g]indazole-3-carboxamide | 179 | >200 |
| 72 | 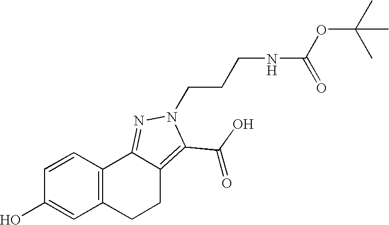 | 2-{3-((tert- butoxycarbonyl)amino]propyl}-7- hydroxy-4,5-dihydro-2H- benzo[g]indazole-3-carboxylic acid | 181 | >200 |
| 73 | 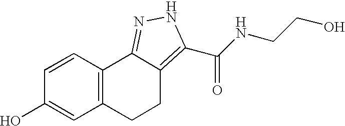 | 7-hydroxy-N-(2-hydroxyethyl)-4,5- dihydro-2H-benzo[g]indazole-3- carboxamide | 184 | >200 |
| 74 | 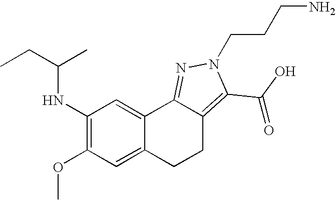 | 2-(3-aminopropyl)-8-(Sec- butylamino)-7-methoxy-4,5-dihydro 2H-benzo[g]indazole-3-carboxylic acid | 196 | |
| 75 | 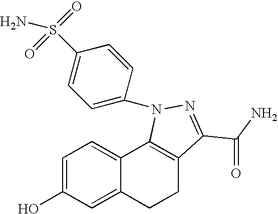 | ethyl 7-methoxy-4,5-dihydro-2H- benzo[g]indazole-3-carboxylate | >200 | >200 |
| 76 | 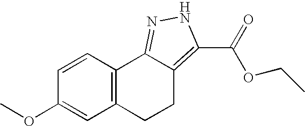 | 7-methoxy-4,5-dihydro-2H- benzo[g]indazole-3-carboxamide | >200 | >200 |
| 77 | 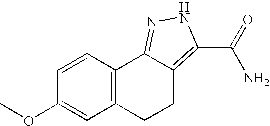 | ethyl 7-hydroxy-1-phenyl-4,5- dihydro-1H-benzo[g]indazole-3- carboxylate | >200 | >200 |
| 78 | 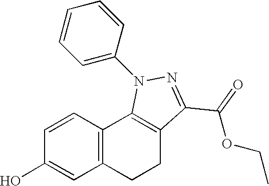 | ethyl 7-hydroxy-1-(4- methoxyphenyl)-4,5-dihydro-1H- benzo[g]indazole-3-carboxylate | >200 | >200 |
| 79 | 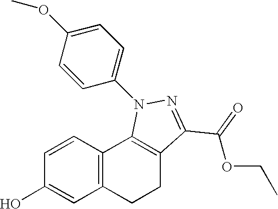 | 2-allyl-7-[(2- methoxyethoxy)methoxy]-4,5- dihydro-2H-benzo[g]indazole-3- carboxamide | >200 | >200 |
| 80 | 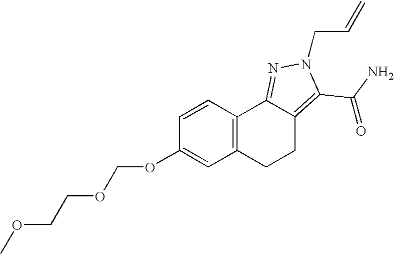 | 2-allyl-7-[(2- methoxyethoxy)methoxy]-4,5- dihydro-2H-benzo[g]indazole-3- carboxylic acid | >200 | >200 |
| 81 | 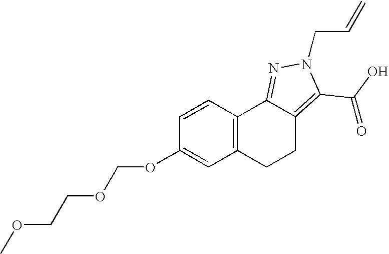 | ethyl 7-methoxy-5-phenyl-4,5- dihydro-1H-benzo[g]indazole-3- carboxylate | >200 | >200 |
| 82 | 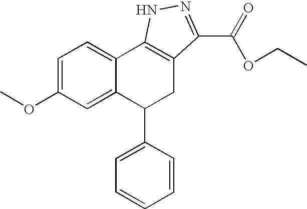 | 7-[(2-methoxyethoxy)methoxy]-4,5- dihydro-2H-benzo[g]indazole-3- carboxamide | >200 | >200 |
| 83 | 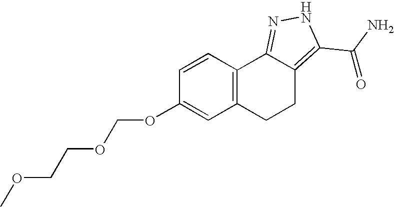 | 6-bromo-7-[(2- methoxyethoxy)methoxy]-4,5- dihydro-2H-benzo[g]indazole-3- carboxamide | >200 | >200 |
| 84 | 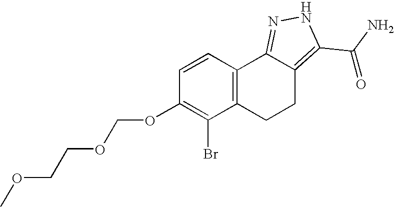 | 6-bromo-7-[(2- methoxyethoxy)methoxy]-4,5- dihydro-2H-benzo[g]indazole-3- carboxylic acid | >200 | >200 |
| 85 | 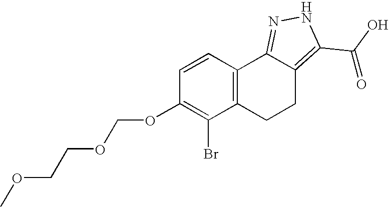 | ethyl 6-bromo-7-[(2- methoxyethoxy)methoxy]-4,5- dihydro-2H-benzo[g]indazole-3- carboxylate | >200 | >200 |
| 86 | 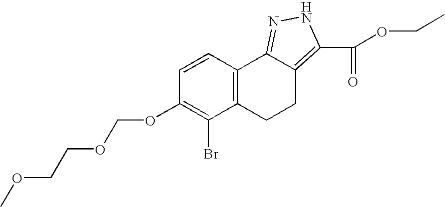 | ethyl 2-allyl-7-[(2- ethoxyethoxy)methoxy]-4,5- dihydro-2H-benzo[g]indazole-3- carboxylate | >200 | >200 |
| 87 | 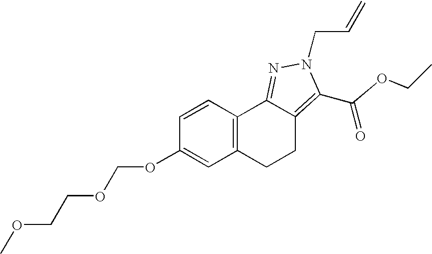 | 7-hydroxy-N,N-dimethyl-4,5- dihydro-2H-benzo[g]indazole-3- carboxamide | >200 | >200 |
| 88 | 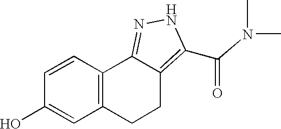 | 8-bromo-2-{2-[(tert- butoxycarbonyl)amino]ethyl}-7- methoxy-4,5-dihydro-2H- benzo[g]indazole-3-carboxylic acid | >200 | >200 |
| 89 | 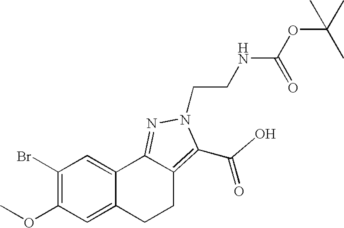 | ethyl 8-bromo-2-{2-[(tert- butoxycarbonyl)amino]ethyl}-7- methoxy-4,5-dihydro-2H- benzo[g]indazole-3-carboxylate | >200 | |
| 90 | 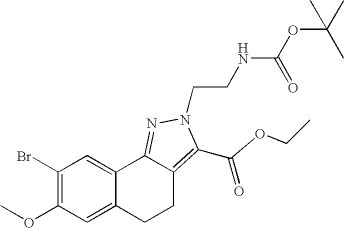 | 8-bromo-2-{3-[(tert- butoxycarbonyl)amino]propyl}-7- methoxy-4,5-dihydro-2H- benzo[g]indazole-3-carboxylic acid | >200 | |
| 91 | 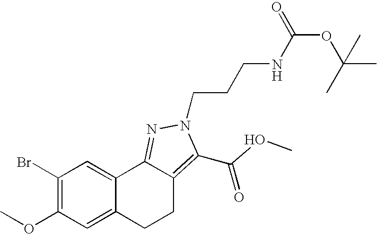 | 8-[(dimethylamino)sulfonyl]-7- hydroxy-4,5-dihydro-2H- benzo[g]indazole-3-carboxylic acid | >200 | |
| 92 | 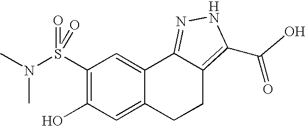 | ethyl 8-bromo-2-{3-[(tert- butoxycarbonyl)amino]propyl}-7- methoxy-4,5-dihydro-2H- benzo[g]indazole-3-carboxylate | >200 | |
| 93 | 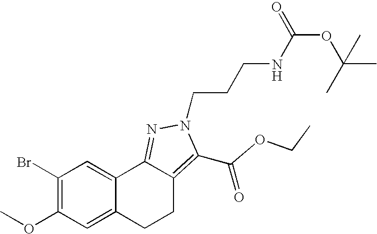 | 7-hydroxy-8- [(isopropylamino)sulfonyl]-4,5- dihydro-2H-benzo[g]indazole-3- carboxylic acid | >200 | |
| 94 | 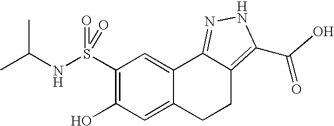 | methyl 7-hydroxy-8- [(isopropylamino)sulfonyl]-4,5- dihydro-2H-benzo[g]indazole-3- carboxylate | >200 | |
| 95 | 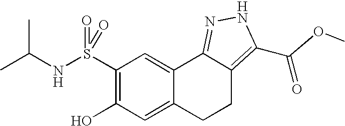 | ethyl 8-bromo-7-methoxy-4,5- dihydro-2H-benzo[g]indazole-3- carboxylate | >200 | |
| 96 | 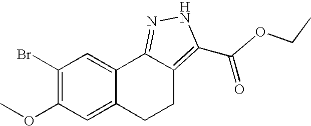 | 8-(aminosulfonyl)-7-hydroxy-4,5- dihydro-2H-benzo[g]indazole-3- carboxylic acid | >200 | |
| 97 | 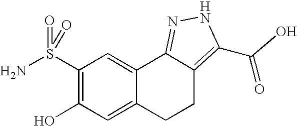 | methyl 7-hydroxy-5-methyl-4,5- dihydro-2H-benzo[g]indazole-3- carboxylate | >200 | |
| 98 | 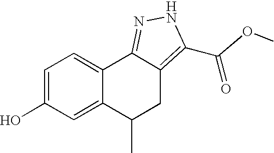 | methyl 7-methoxy-5-methyl-4,5- dihydro-2H-benzo[g]indazole-3- carboxylate | >200 | |
| 99 | 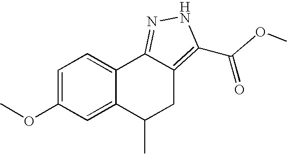 | methyl 7-methoxy-4-methyl-4,5- dihydro-2H-benzo[g]indazole-3- carboxylate | >200 | |
| 100 | 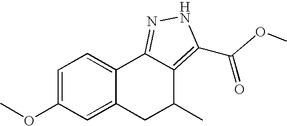 | ethyl 8-(aminosulfonyl)-7-methoxy- 4,5-dihydro-2H-benzo[g]indazole-3- carboxylate | >200 | |
| 101 | 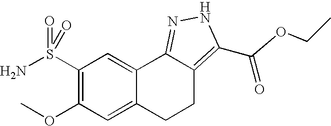 | ethyl 2-(3-aminopropyl)-7-hydroxy- 4,5-dihydro-2H-benzo[g]indazole-3- carboxylate dihydrochloride | >200 | |
| 102 | 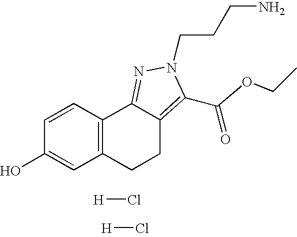 | ethyl 7-[(2- methoxyethoxy)methoxy]-4,5- dihydro-2H-benzo[g]indazole-3- carboxylate | >200 | >200 |
| 103 | 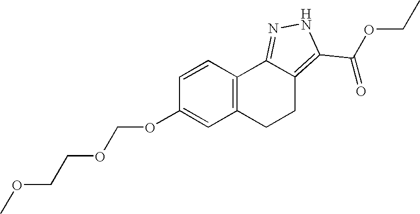 | 5-butyl-7-hydroxy-4,5-dihydro-2H- benzo[g]indazole-3-carboxamide | >200 | >200 |
| 104 | 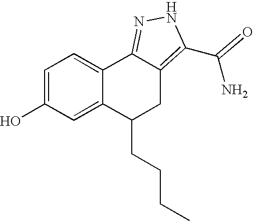 | 5-ethyl-7-hydroxy-4,5-dihydro-2H- benzo(g]indazole-3-carboxamide | >200 | >200 |
| 105 | 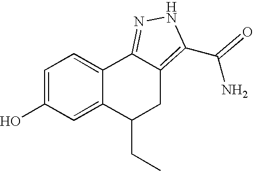 | 5-butyl-7-hydroxy-4,5-dihydro-2H- benzo[g]indazole-3-carboxylic acid | >200 | >200 |
| 106 | 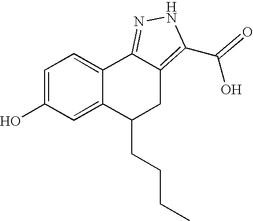 | 5-ethyl-7-hydroxy-4,5-dihydro-2H- benzo[g]indazole-3-carboxylic acid | >200 | >200 |
| 107 | 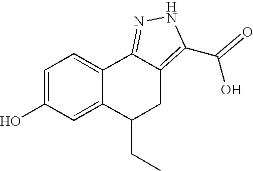 | 5-benzyl-7-hydroxy-4,5-dihydro-1H- benzo[g]indazole-3-carboxamide | >200 | >200 |
| 108 | 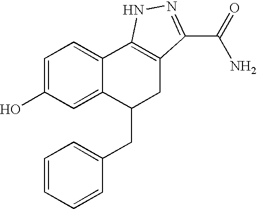 | 7-hydroxy-5-phenyl-4,5-dihydro-1H benzo[g]indazole-3-carboxamide | >200 | >200 |
| 109 | 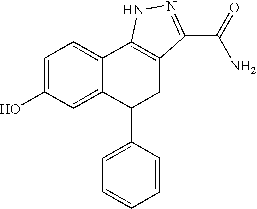 | 5-benzyl-7-hydroxy-4,5-dihydro-1H benzo[g]indazole-3-carboxylic acid | >200 | >200 |
| 110 | 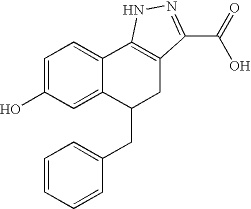 | ethyl 5-allyl-7-methoxy-4,5-dihydro- 1H-benzo[g]indazole-3-carboxylate | >200 | >200 |
| 111 | 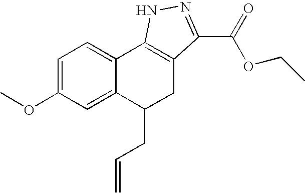 | ethyl 5-benzyl-7-methoxy-4,5- dihydro-1H-benzo[g]indazole-3- carboxylate | >200 | >200 |
| 112 | 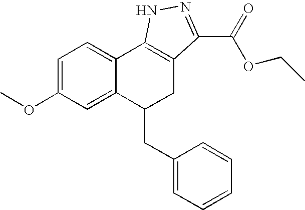 | 7-hydroxy-5-phenyl-4,5-dihydro-1H benzo[g]indazole-3-carboxyIic acid | >200 | >200 |
| 113 | 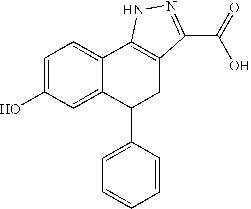 | | >200 | >200 |
| 114 | 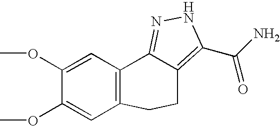 | methyl 2-{3-[(tert- butoxycarbonyl)amino]propyl}-8- (3,4-difluorophenyl)-7-methoxy-4,5- dihydro-2H-benzo[g]indazole-3- carboxylate | >200 | >200 |
| 115 | 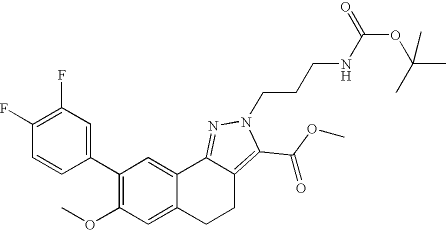 | methyl 2-{3-[(tert- butoxycarbonyl)amino]propyl}-7- methoxy-8-[3- (methylsulfonyl)phenyl]-4,5-dihydro 2H-benzo[g]indazole-3-carboxylate | >200 | |
| 116 | 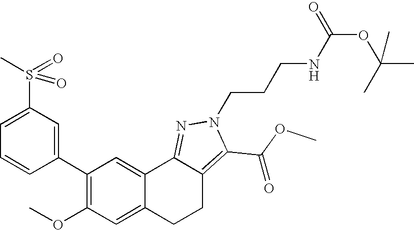 | methyl 2-{3-[(tert- butoxycarbonyl)amino]propyl}-7- methoxy-8-(4-nitrophenyl)-4,5- dihydro-2H-benzo[g]indazole-3- carboxylate | >200 | |
| 117 | 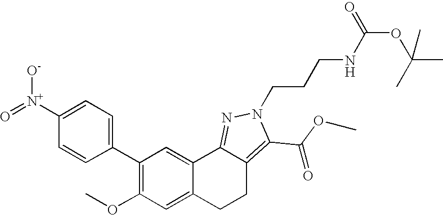 | methyl 2-{3-[(tert- butoxycarbonyl)amino]propyl}-7- methoxy-8-(3,3,3-trifluoropropyl)- 4,5-dihydro-2H-benzo[g]indazole-3- carboxylate | >200 | |
| 118 | 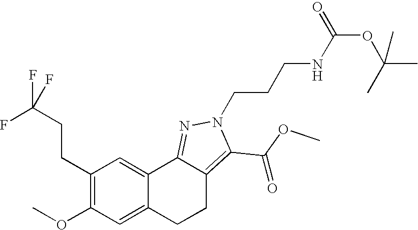 | 3-methoxy-2-(3-nitrophenyl)- 5,6,9,10- tetrahydrobenzo[g]pyraziflo[1,2- b]indazol-7(8H)-one | >200 | |
| 119 | 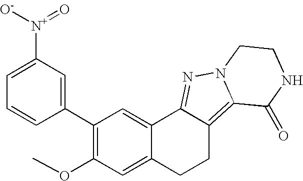 | methyl 2-{3-[(tert- butoxycarbonyl)amino]propyl}-8-(3- cyanophenyl)-7-methoxy-4,5- dihydro-2H-benzo[g]indazole-3- carboxylate | >200 | |
| 120 | 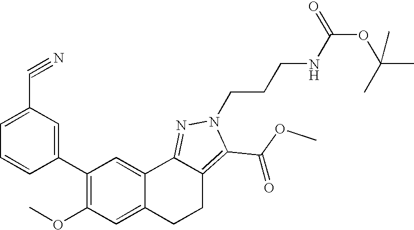 | methyl 2-{2-[(tert- butoxycarbonyl)amino]ethyl}-7- methoxy-8-(3-nitrophenyl)-4-5- dihydro-2H-benzo[g]indazole-3- carboxylate | >200 | |
| 121 | 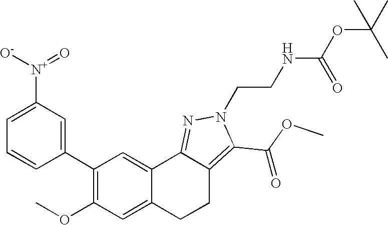 | 2-amino-3-methoxy-5,6,8,9,10,11- hexahydro-7H- benzo[g][1,4]diazepino[1,2- b]indazol-7-one | >200 | |
| 122 | 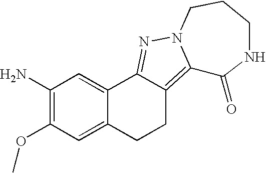 | methyl 8-amino-2-(3-aminopropyl)- 7-methoxy-4,5-dihydro-2H- benzo[g]indazole-3-carboxylate hydrochloride | >200 | |
| 123 | 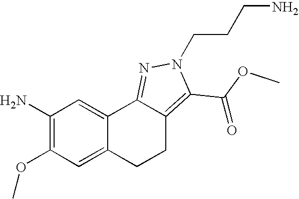 | 2-{3-[(tert- butoxycarbonyl)amino]propyl}-7- methoxy-8-(5-nitropentyl)-4,5- dihydro-2H-benzo[g]indazole-3- carboxylic acid | >200 | |
| 124 | 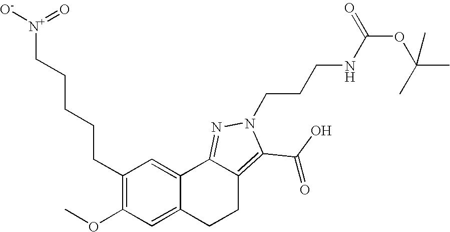 | 2-{3-[(tert- butoxycarbonyl)amino]propyl}8-(4- carboxy-3,3-dimethylbutyl)-7- methoxy-4,5-dihydro-2H- benzo[g]indazole-3-carboxylic acid | >200 | |
| 125 | 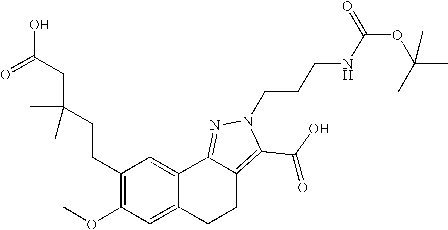 | 2-{3-[(tert- butoxycarbonyl)amino]propyl}8- (3,5-dimethylisoxazol-4-yl)-7- methoxy-4,5-dihydro-2H- benzo-[g]indazoIe-3-carboxyIic acid | >200 | |
| 126 | 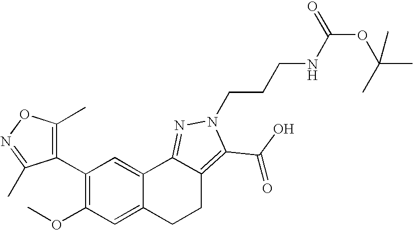 | methyl 2-{3-[(tert- butoxycarbonyl)amino]propyl}-8- (3,5-dimethylisoxazol-4-yl)-7- methoxy-4,5-dihydro-2H- benzo[g]indazole-3-carboxylate | >200 | |
| 127 | 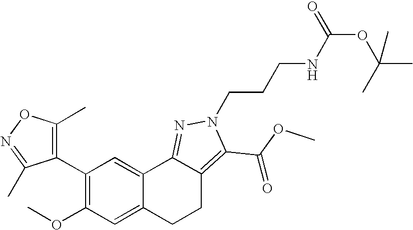 | 2-{3-[(tert- butoxycarbonyl)amino]propyl}-8-(3- carboxyphenyl)-7-methoxy-4,5- dihydro-2H-benzo[g]indazoIe-3- carboxylic acid | >200 | |
| 128 | 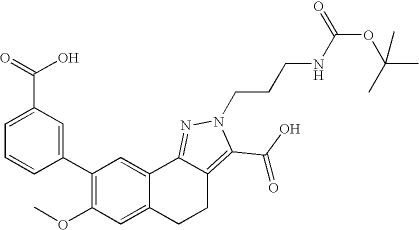 | methyl 2-{3-[(tert- butoxycarbonyl)amino]propyl}-7- methoxy-8-[3- (methoxycarbonyl)phenyl]-4,5- dihydro-2H-benzo[g]indazole-3- carboxylate | >200 | |
| 129 | 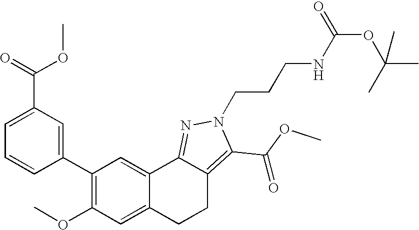 | 2-{3-[(tert- butoxycarbonyl)amino]propyl}-8- isobutyl-7-methoxy-4,5-dihydro-2H- benzo[g]indazole-3-carboxylic acid | >200 | |
| 130 | 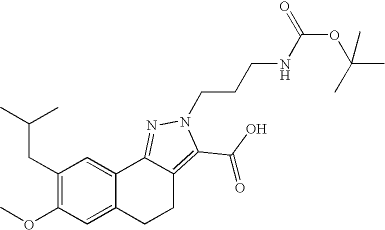 | methyl 2-{3-[(tert- butoxycarbonyl)amino]propyll-8-(3- chlorophenyl)-7-methoxy-4,5- dihydro-2H-benzo[g]indazole-3- carboxylate | >200 | |
| 131 | 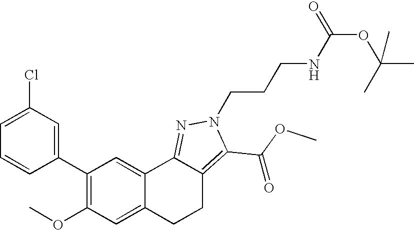 | methyl 2-{3-[(tert- butoxycarbonyl)amino]propyl}-8- (sec-butylamino)-7-methoxy-4,5- dihydro-2H-benzo[g]indazole-3- carboxylate | >200 | |
| 132 | 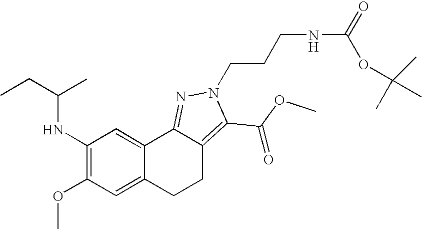 | methyl 2-{3-[(tert- butoxycarbonyl)amino]propyl}-8- isobutyl-7-methoxy-4,5-dihydro-2H- benzo[g]indazole-3-carboxylate | >200 | |
| 133 | 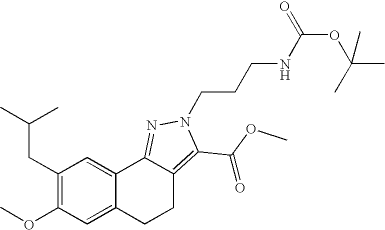 | methyl 8-benzyl-2-{3-[(tert- butoxycarbonyl)amino]propyl}-7- methoxy-4,5-dihydro-2H- benzo[g]indazole-3-carboxylate | >200 | |
| 134 | 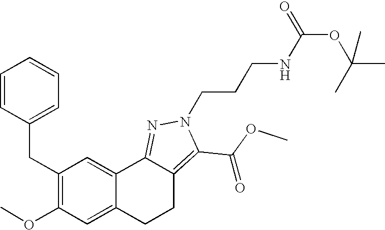 | 3-methoxy-2-(3-nitrophenyl)- 5,6,8,9,10,11-hexahydro-7H- benzo[g][1,4]diazepino[1,2- b]indazol-7-one | >200 | |
| 135 | 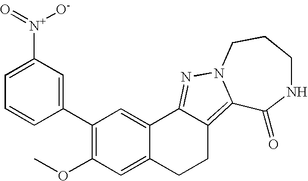 | 2-bromo-3-methoxy-5,6,9,10- tetrahydrobenzol[g]pyrazino[1,2- b]indazol-7(8H)-one | >200 | |
| 136 | 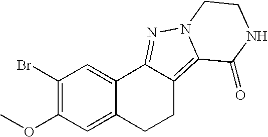 | methyl 7-methoxy-6,8-dinitro-4,5- dihydro-2H-benzo[g]indazole-3- carboxylate | >200 | |
| 137 | 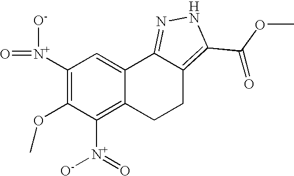 | methyl 7-methoxy-8-nitro-4,5- dihydro-2H-benzo[g]indazole-3- carboxylate | >200 | |
| 138 | 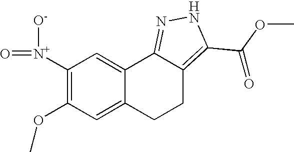 | methyl 2-{3-[(tert- butoxycarbonyl)amino]propyl}-7- methoxy-8-nitro-4,5-dihydro-2H- benzo[g]indazole-3-carboxylate | >200 | |
| 139 | 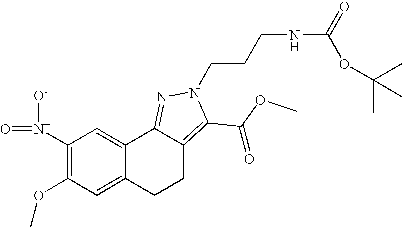 | methyl 8-amino-2-{3-[(tert- butoxycarbonyl)amino]propyl}-7- methoxy-4,5-dihydro-2H- benzo[g]indazole-3-carboxylate | >200 | |
| 140 | 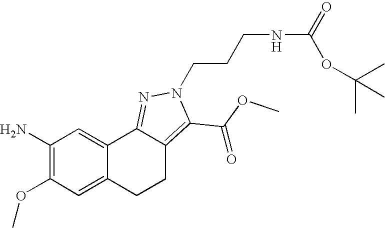 | 8-amino-2-(3-aminopropyl)-7- methoxy-4,5-dihydro-2H- benzo[g]indazole-3-carboxylic acid | >200 | |
| 141 | 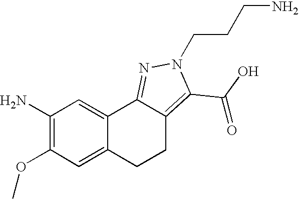 | methyl 2-{3-[(tert- butoxycarbonyl)amino]propyl}-8-[2- (4-chlorophenyl)ethyl]-7-methoxy- 4,5-dihydro-2H-benzo[g]indazoIe-3- carboxylate | >200 | |
| 142 | 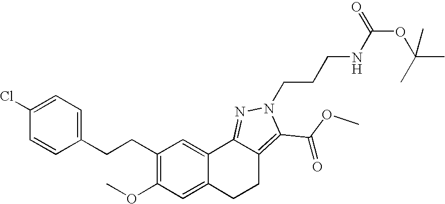 | methyl 2-{3-{(tert- butoxycarbonyl)amino]propyl}-8-[1- (tert-butoxycarbonyl)-1H-pyrrol-2- yl]-7-methoxy-4,5-dihydro-2H- benzo[g]indazole-3-carboxylate | >200 | |
| 143 | 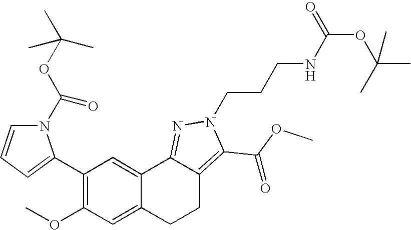 | 2-{3-[(tert- butoxycarbonyl)amino]propyl}-7- methoxy-5-methyl-4,5-dihydro-2H- benzo[g]indazole-3-carboxylic acid | >200 | |
| 144 | 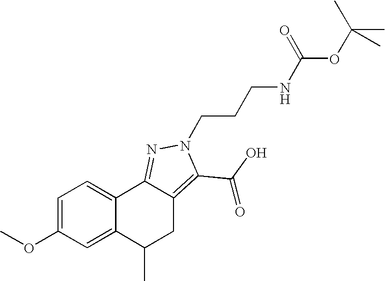 | methyl 2-{3-[(tert- butoxycarbonyl)amino]propyl}-8-(2- chlorophenyl)-7-methoxy-4,5- dihydro-2H-benzo[g]indazole-3- carboxylate | >200 | |
| 145 | 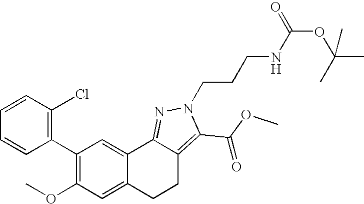 | 2-(3-aminopropyl)-8-(4- chlorophenyl)-7-methoxy-4,5- dihydro-2H-benzo[g]indazole-3- carboxylic acid hydrochloride | >200 | |
| 146 | 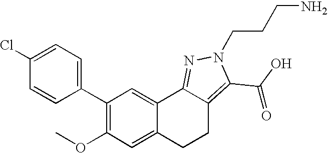 | methyl 2-{3-[(tert- butoxycarbonyl)amino]propyl}-7- methoxy-8-(3-nitrophenyl)-4,5- dihydro-2H-benzo[g]indazole-3- carboxylate | >200 | |
| 147 | 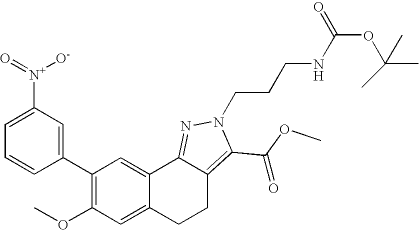 | methyl 2-{3-[(tert- butoxycarbonyl)amino]propyl}-5-(4- tert-butylphenyl)-7-methoxy-4,5- dihydro-2H-benzo[g]indazole-3- carboxylate | >200 | |
| 148 | 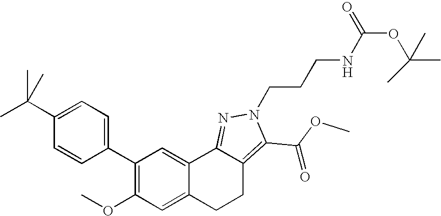 | methyl 2-{3-[(tert- butoxycarbonyl)amino]propyl}-7- methoxy-8-[3- (trifluoromethyl)phenyll-4,5-dihydro 2H-benzo[g]indazole-3-carboxylate | >200 | |
| 149 | 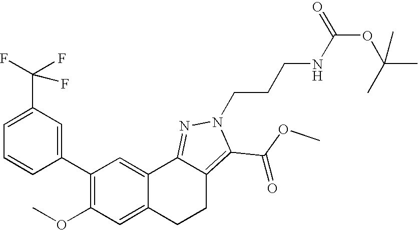 | methyl 2-{3-[(tert- butoxycarbonyl)amino]propyl}-8-(4- chlorophenyl)-7-methoxy-4,5- dihydro-2H-benzo[g]indazole-3- carboxylate | >200 | |
| 150 | 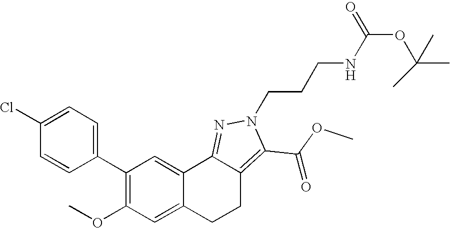 | 2-bromo-3-methoxy-9-methyl- 5,6,9,10- tetrahydrobenzo[g]pyrazino[1,2- b]indazol-7(8H)-one | >200 | |
| 151 | 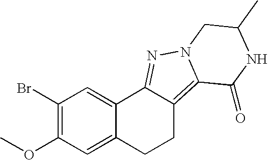 | 2-bromo-3-hydroxy-9-methyl- 5,6,9,10- tetrahydrobenzo[g]pyrazino[1,2- b]indazol-7(8H)-one | >200 | >200 |
| 152 | 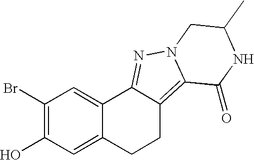 | methyl 2-{3-[(tert- butoxycarbonyl)amino]propyl}-8-[4- (dimethylamino)phenyl]-7-methoxy- 4,5-dihydro-2H-benzo[9]indazole-3- carboxylate | >200 | >200 |
| 153 | 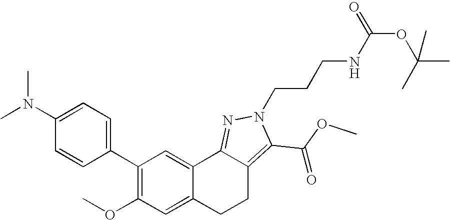 | 2-(2-aminoethyl)-8-bromo-7- methoxy-4,5-dihydro-2H- benzo[g]indazole-3-carboxylic acid hydrochloride | >200 | |
| 154 | 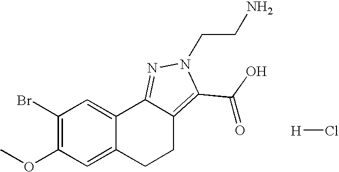 | ethyl 7-hydroxy-1-methyl-4,5- dihydro-1H-benzo[g]indazole-3- carboxylate | >200 | |
| 155 | 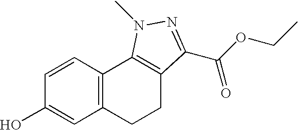 | 7-hydroxy-1-phenyl-4,5-dihydro-1H benzo[g]indazole-3-carboxamide | >200 | >200 |
| 156 | 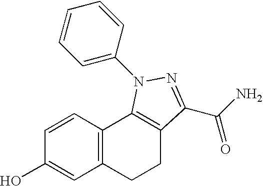 | ethyl 7-hydroxy-2-methyl-4,5- dihydro-2H-benzo[g]indazole-3- carboxylate | >200 | >200 |
| 157 | 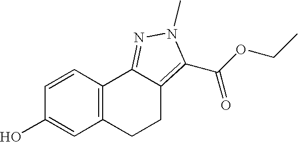 | | >200 | >200 |
| 158 | 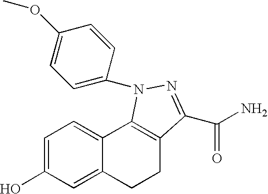 | | >200 | >200 |
| 159 | 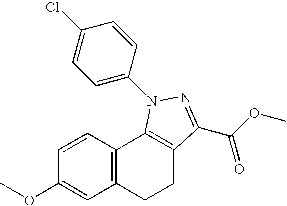 | | >200 | >200 |
| 160 | 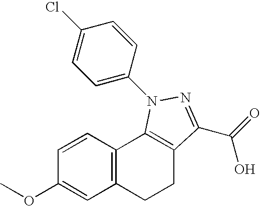 | | >200 | >200 |
| 161 | 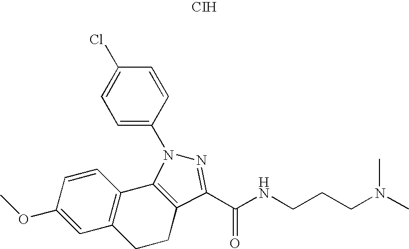 | | >200 | >200 |
| 162 | 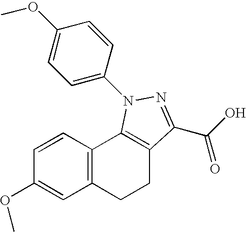 | | >200 | >200 |
| 163 | 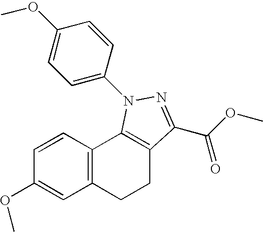 | | >200 | >200 |
| 164 | 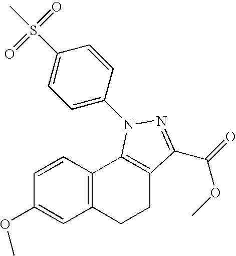 | | >200 | >200 |
| 165 | 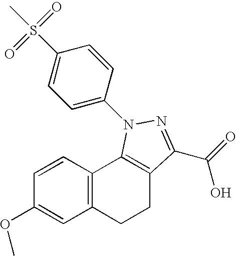 | | >200 | >200 |
| 166 | 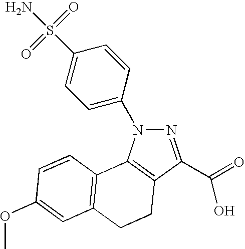 | | >200 | >200 |
| 167 | 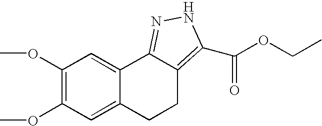 | | >200 | >200 |
| 168 | 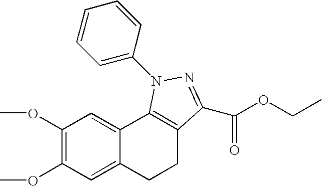 | | >200 | >200 |
| 169 | 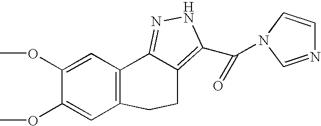 | | >200 | >200 |
| 170 | 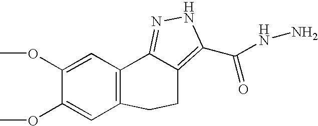 | | >200 | >200 |
| 171 | 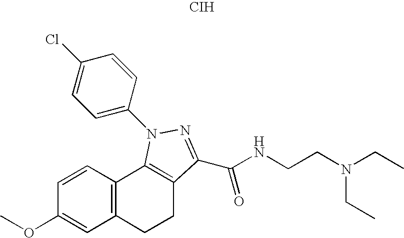 | | >200 | >200 |
| 172 | 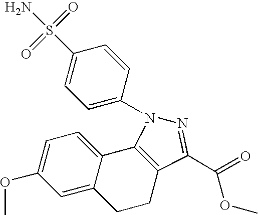 | | >200 | >200 |
| 173 | 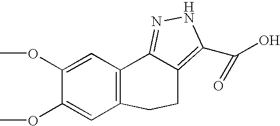 | | >200 | >200 |
| 174 | 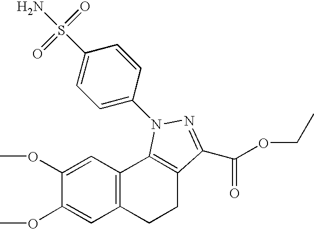 | | >200 | >200 |
| 175 | 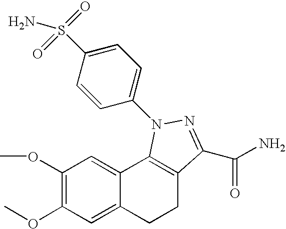 | | >200 | >200 |
| 176 | 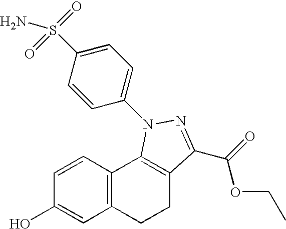 | | >200 | >200 |
| 177 | 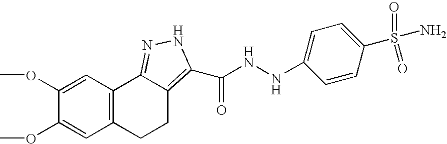 | | >200 | >200 |
| 178 | 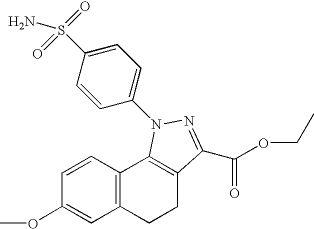 | | >200 | >200 |
| 179 | 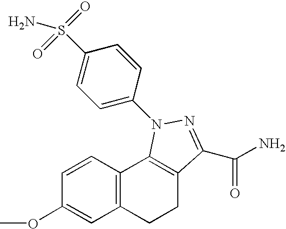 | | >200 | >200 |
| 180 | 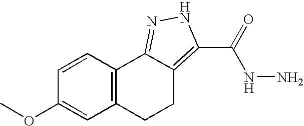 | | >200 | >200 |
| 181 | 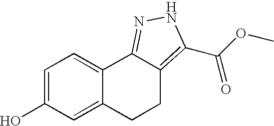 | | >200 | >200 |
| 182 | 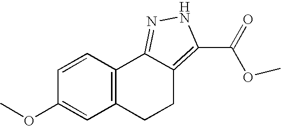 | | >200 | >200 |
| 183 | 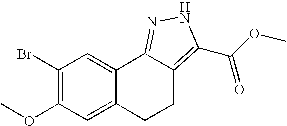 | | >200 | >200 |
| 184 | 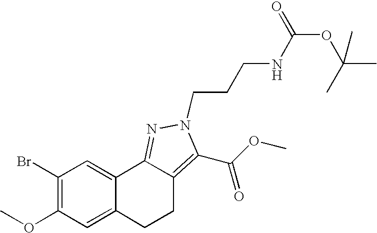 | | >200 | >200 |
| 185 | 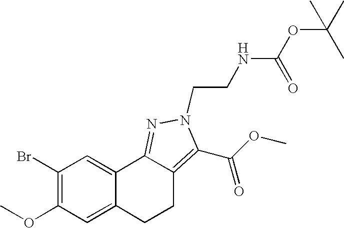 | | >200 | >200 |
| 186 | 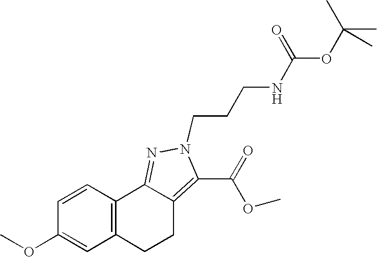 | | >200 | >200 |

^{a}The pyrazolylpyrazole compound may be shown with a solvent, such as, for example, trifluoroacetate, with which it can form a salt. Both the salt and acid forms of the pyrazolylpyrazole compound are included in the present invention.
^{b}compound names generated by ACD/Name software.

TABLE 2
Pyrazolylpyrazole MK-2 inhibitors

| | | | MK-2 | MK-3 |
|---|---|---|---|---|
| | | | Avg. | Avg. |
| | | | IC50 | IC50 |
| No. | Structure^{a} | Compound Name^{b} | (uM) | (uM) |
| 1 | 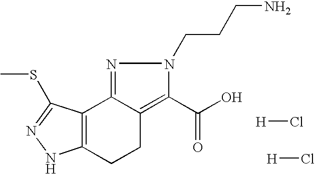 | 2-(3-aminopropyl)-8-(methylthio) 2,4,5,6-tetrahydropyrazolo[3,4- e]indazole-3-carboxylic acid dihydrochloride | 1.06 | 47 |
| 2 | 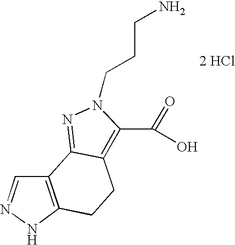 | 2-(3-aminopropyl)-2,4,5,6- tetrahydropyrazolo[3,4- e]indazole-3-carboxylic acid hydrochloride | 2.17 | 35.3 |
| 3 | 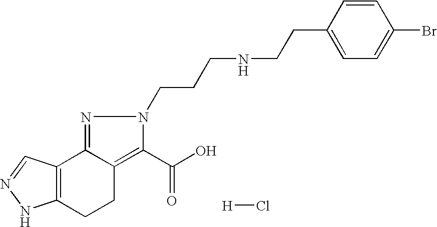 | 2-(3-{[2-(4- bromophenyl)ethyl]amino}propyl)- 2,4,5,6-tetrahydropyrazolo[3,4- e]indazole-3-carboxylic acid hydrochloride | 2.23 | 20.8 |
| 4 | 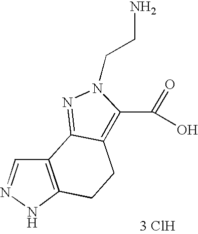 | 2-(2-aminoethyl)-2,4,5,6- tetrahydropyrazolo[3,4- e]indazole-3-carboxylic acid hydrochloride | 2.43 | 63.3 |
| 5 | 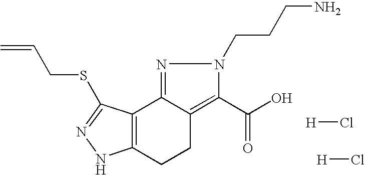 | 8-(allylthio)-2-(3-aminopropyl)- 2,4,5,6-tetrahydropyrazolo[3,4- e]indazole-3-carboxylic acid dihydrochloride | 2.84 | 41.4 |
| 6 | 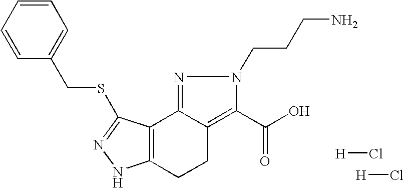 | 2-(3-aminopropyl)-8-(benzylthio) 2,4,5,6-tetrahydropyrazolo[3,4- e]indazole-3-carboxylic acid dihydrochloride | 2.89 | 22.1 |
| 7 | 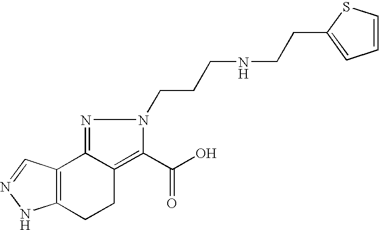 | 2-{3-[(2-thien-2- ylethyl)amino]propyl}-2,4,5,6- tetrahydropyrazolo[3,4- e]indazole-3-carboxylic acid | 3.66 | 55.2 |
| 8 | 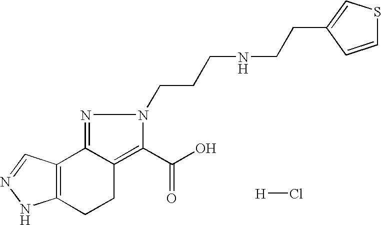 | 2-{3-[(2-thien-3- ylethyl)amino]propyl}-2,4,5,6- tetrahydropyrazolo[3,4- e]indazole-3-carboxylic acid hydrochloride | 7.84 | 48.5 |
| 9 | 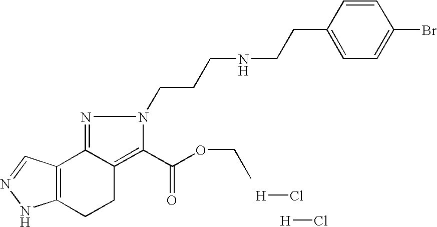 | ethyl 2-(3-{[2-(4- bromophenyl)ethyl]amino}propyl)- 2,4,5,6-tetrahydropyrazolo[3,4- e]indazole-3-carboxylate | 8.54 | 68.1 |
| 10 | 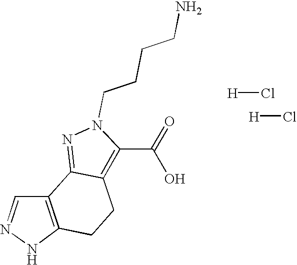 | 2-(2-aminobutyl)-2,4,5,6- tetrahydropyrazolo[3,4- e]indazole-3-carboxylic acid dihydrochloride | 10.1 | 200 |
| 11 | 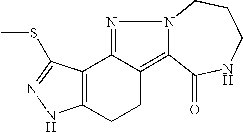 | 1-(methylthio)-4,5,7,8,9,10- hexahydro[1,4]diazepino[1,2- b]pyrazolo[3,4-g]indazol-6(3H)- one | 10.5 | 200 |
| 12 | 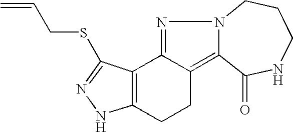 | 1-(allylthio)-4,5,7,8,9,10- hexahydro[1,4]diazepino[1,2- b]pyrazolo[3,4-g]indazol-6(3H)- one | 10.8 | 55.6 |
| 13 | 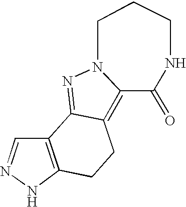 | 4,5,7,8,9,10- hexahydro[1,4]diazepino[1,2- b]pyrazolo[3,4-g]indazol-6(3H)- one | 19.6 | 144 |
| 14 | 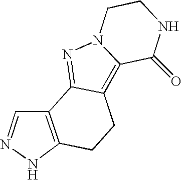 | 4,5,8,9-tetrahydro-3H- pyrazino[1,2-b]pyrazolo[3,4- g]indazol-6(7H)-one | 20 | 137 |
| 15 | 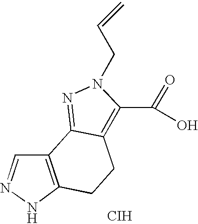 | 2-allyl-2,4,5,6- tetrahydropyrazolo[3,4- e]indazole-3-carboxylic acid hydrochloride | 22.7 | 134 |
| 16 | 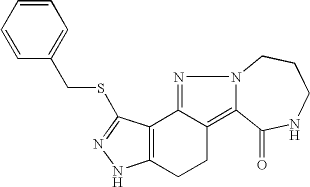 | 1-(benzylthio)-4,5,7,8,9,10- hexahydro[1,4]diazepino[1,2- b]pyrazolo[3,4-g]indazol-6(3H)- one | 23.8 | 56 |
| 17 | 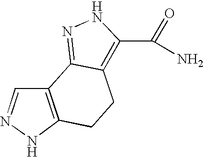 | 2,4,5,6-tetrahydropyrazolo[3,4- e]indazole-3-carboxamide | 28.4 | 129 |
| 18 | 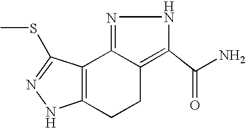 | 8-(methylthio)-2,4,5,6- tetrahydropyrazolo[3,4- e]indazole-3-carboxamide | 30.3 | |
| 19 | 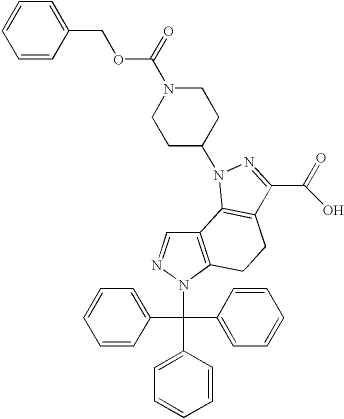 | 1-{1- [(benzyloxy)carbonyl]piperidin-4- yl}-6-trityl-1,4,5,6- tetrahydropyrazolo[3,4- e]indazole-3-carboxylic acid | 46.5 | 200 |
| 20 | 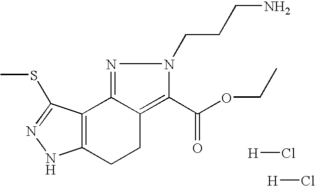 | ethyl 2-(3-aminopropyl)-8- (methylthio)-2,4,5,6- tetrahydropyrazolo[3,4- e]indazole-3-carboxylate dihydrochloride | 55.3 | 200 |
| 21 | 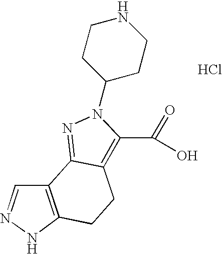 | 2-piperidin-4-yl-2,4,5,6- tetrahydropyrazolo[3,4- e]indazole-3-carboxylic acid hydrochloride | 60.4 | 200 |
| 22 | 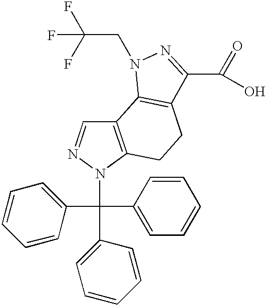 | 1-(2,2,2-trifluoroethyl)-6-trityl- 1,4,5,6-tetrahydropyrazolo[3,4- e]indazole-3-carboxylic acid | 68.3 | 195 |
| 23 | 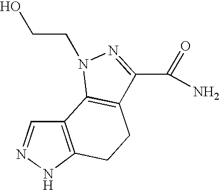 | 1-(2-hydroxyethyl)-1,4,5,6- tetrahydropyrazolo[3,4- e]indazole-3-carboxamide | 72.2 | 200 |
| 24 | 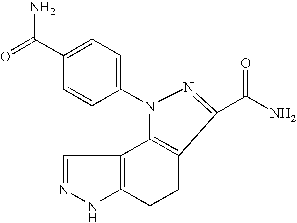 | 1-[4-(aminocarbonyl)phenyl]- 1,4,5,6-tetrahydropyrazolo[3,4- e]indazole-3-carboxamide or 1- [4-(aminocarbonyl)phenyl]- 1,4,5,6-tetrahydropyrazolo[3,4- e]indazole-3-carboxamide | 78.4 | 200 |
| 25 | 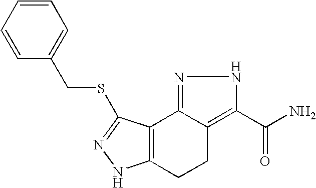 | 8-(benzylthio)-2,4,5,6- tetrahydropyrazolo[3,4- e]indazole-3-carboxamide | 80.7 | |
| 26 | 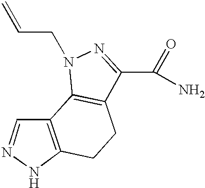 | 1-allyl-1,4,5,6- tetrahydropyrazolo[3,4- e]indazole-3-carboxamide | 95.1 | 49.3 |
| 27 | 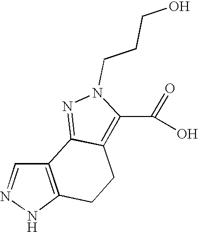 | 2-(3-hydroxypropyl)-2,4,5,6- tetrahydropyrazolo[3,4- e]indazole-3-carboxylic acid | 103 | 200 |
| 28 | 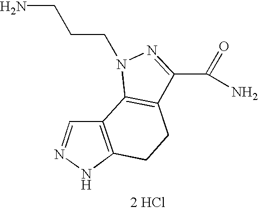 | 1-(3-aminopropyl)-1,4,5,6- tetrahydropyrazolo[3,4- e]indazole-3-carboxamide dihydrochloride | 104 | |
| 29 | 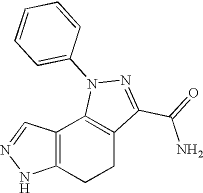 | 1-phenyl-1,4,5,6- tetrahydropyrazolo[3,4- e]indazole-3-carboxmide | 112 | 200 |
| 30 | 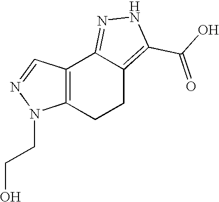 | 6-(2-hydroxyethyl)-2,4,5,6- tetrahydropyrazolo[3,4- e]indazole-3-carboxylic acid | 114 | 200 |
| 31 | 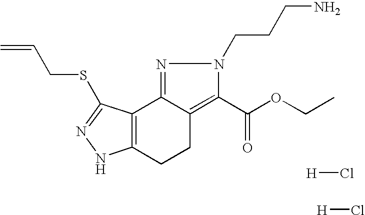 | | 114 | 200 |
| 32 | 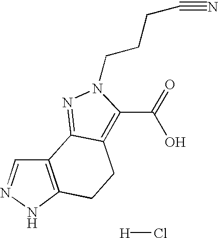 | 2-(3-cyanopropyl)-2,4,5,6- tetrahydropyrazolo[3,4- e]indazole-3-carboxylic acid hydrochloride | 116 | 200 |
| 33 | 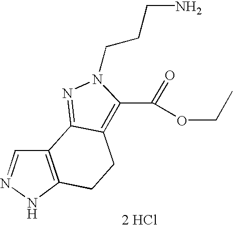 | ethyl 2-(3-aminopropyl)-2,4,5,6- tetrahydropyrazolo[3,4- e]indazole-3-carboxylate dihydrochloride | 116 | 186 |
| 34 | 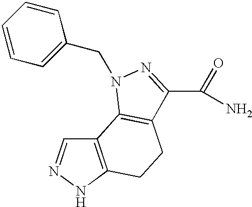 | 1-benzyl-1,4,5,6- tetrahydropyrazolo[3,4- e]indazole-3-carboxamide | 120 | 200 |
| 35 | 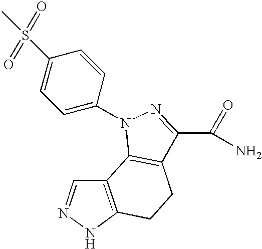 | 1-[4-(methylsulfonyl)phenyl]- 1,4,5,6-tetrahydropyrazolo[3,4- e]indazole-3-carboxamide | 124 | 200 |
| 36 | 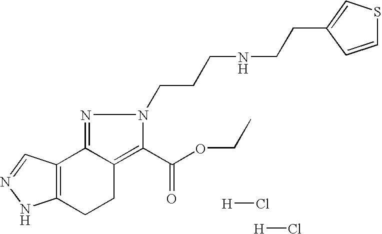 | ethyl 2-{3-[(2-thien-3- ylethyl)amino]propyl}-2,4,5,6- tetrahydropyrazolo[3,4- e]indazole-3-carboxylate dihydrochloride | 133 | 200 |
| 37 | 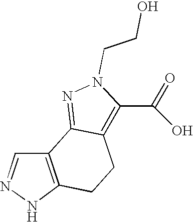 | 2-(2-hydroxyethyl)-2,4,5,6- tetrahydropyrazolo[3,4- e]indazole-3-carboxylic acid | 139 | 200 |
| 38 | 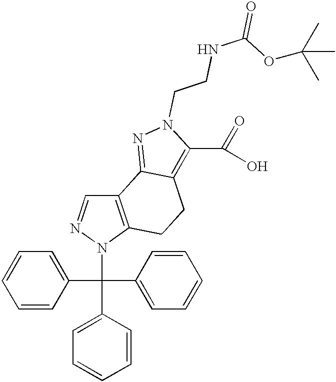 | 2-{2-[(tert- butoxycarbonyl)amino]ethyl}-6- trityl-2,4,5,6- tetrahydropyrazolo[3,4- e]indazole-3-carboxylic acid | 139 | 200 |
| 39 | 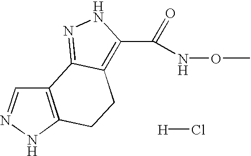 | N-methoxy-2,4,5,6- tetrahydropyrazolo[3,4- e]indazole-3-carboxamide hydrochloride | 141 | 180 |
| 40 | 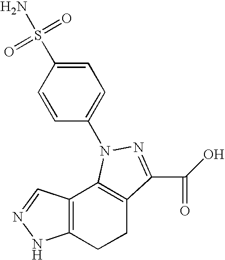 | 1-[4-(aminosulfonyl)phenyl]- 1,4,5,6-tetrahydropyrazolo[3,4- e]indazole-3-carboxylic acid | 145 | 200 |
| 41 | 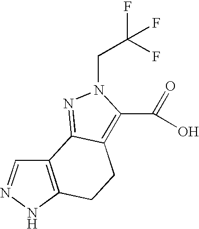 | 2-(2,2,2-trifluoroethyl)-2,4,5,6- tetrahydropyrazolo[3,4- e]indazole-3-carboxylic acid | 150 | 200 |
| 42 | 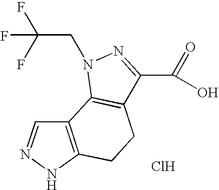 | 1-(2,2,2-trifluoroethyl)-1,4,5,6- tetrahydropyrazolo[3,4- e]indazole-3-carboxylic acid hydrochloride | 180 | 200 |
| 43 | 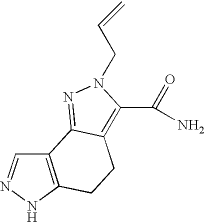 | 2-allyl-2,4,5,6- tetrahydropyrazolo[3,4- e]indazole-3-carboxmide | 181 | 200 |
| 44 | 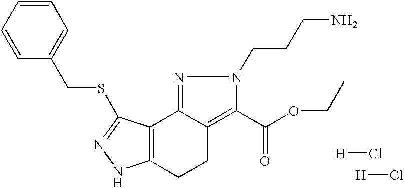 | ethyl 2-(3-aminopropyl)-8- (benzylthio)-2,4,5,6- tetrahydropyrazolo[3,4- e]indazole-3-carboxylate dihydrochloride | 181 | 200 |
| 45 | 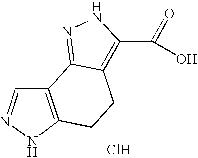 | 2,4,5,6-tetrahydropyrazolo[3,4- e]indazole-3-carboxylic acid hydrochloride | 188 | 200 |
| 46 | 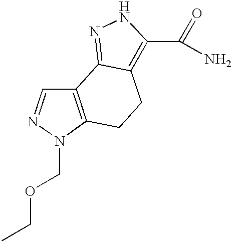 | 6-(ethoxymethyl)-2,4,5,6- tetrahydropyrazolo[3,4- e]indazole-3-carboxamide | 200 | 145 |
| 47 | 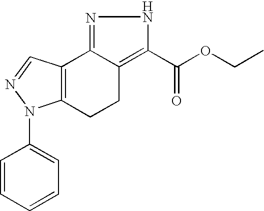 | ethyl 6-phenyl-2,4,5,6- tetrahydropyrazolo[3,4- e]indazole-3-carboxylate | 200 | 200 |
| 48 | 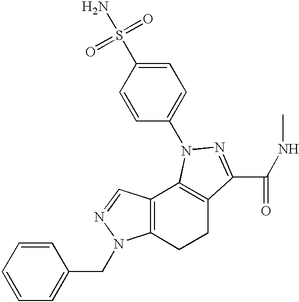 | 1-[4-(aminosulfonyl)phenyl]-6- benzyl-N-methyl-1,4,5,6- tetrahydropyrazolo[3,4- e]indazole-3-carboxamide | 200 | 200 |
| 49 | 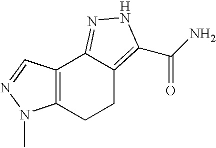 | 6-methyl-2,4,5,6- tetrahydropyrazolo[3,4- e]indazole-3-carboxamide | 200 | 200 |
| 50 | 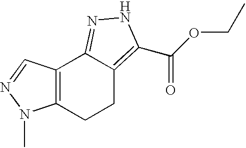 | ethyl 6-methyl-2,4,5,6- tetrahydropyrazolo[3,4- e]indazole-3-carboxylate | 200 | 200 |
| 51 | 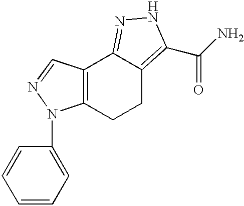 | 6-phenyl-2,4,5,6- tetrahydropyrazolo[3,4- e]indazole-3-carboxamide | 200 | 200 |
| 52 | 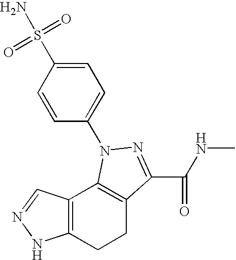 | 1-[4-(aminosulfonyl)phenyl]-N- methyl-1,4,5,6- tetrahydropyrazolo[3,4- e]indazole-3-carboxamide | 200 | 200 |
| 53 | 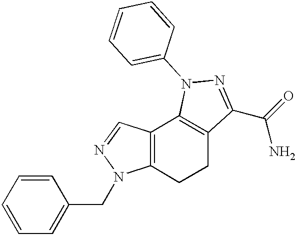 | 6-benzyl-1-phenyl-1,4,5,6- tetrahydropyrazolo[3,4- e]indazole-3-carboxamide | 200 | 200 |
| 54 | 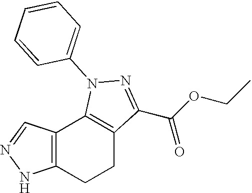 | ethyl 1-phenyl-1,4,5,6- tetrahydropyrazolo[3,4- e]indazole-3-carboxylate | 200 | 200 |
| 55 | 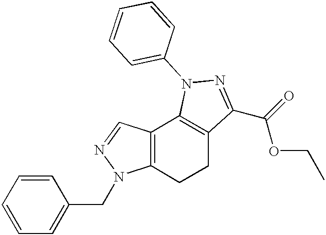 | ethyl 6-benzyl-1-phenyl-1,4,5,6- tetrahydropyrazolo[3,4- e]indazole-3-carboxylate | 200 | 200 |
| 56 | 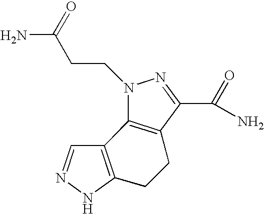 | 1-(3-amino-oxopropyl)-1,4,5,6- tetrahydropyrazolo[3,4- e]indazole-3-carboxamide | 200 | |
| 57 | 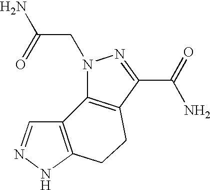 | 1-(2-amino-2-oxoethyl)-1,4,5,6- tetrahydropyrazolo[3,4- e]indazole-3-carboxamide | 200 | |
| 58 | 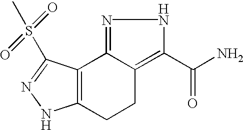 | 8-(methylsulfonyl)-2,4,5,6- tetrahydropyrazolo[3,4- e]indazole-3-carboxamide | 200 | |
| 59 | 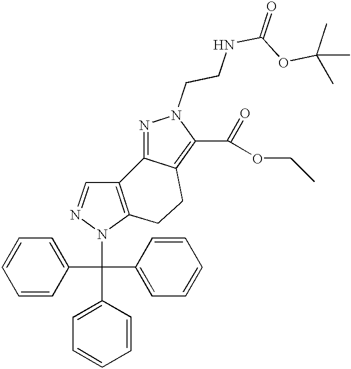 | ethyl 2-{2-[(tert- butoxycarbonyl)amino]ethyl}-6- trityl-2,4,5,6- tetrahydropyrazolo[3,4- e]indazole-3-carboxylate | 200 | 200 |
| 60 | 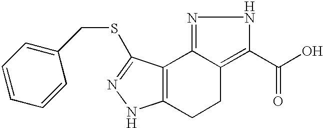 | 8-(benzylthio)-2,4,5,6- tetrahydropyrazolo[3,4- e]indazole-3-carboxylic acid | 200 | |
| 61 | 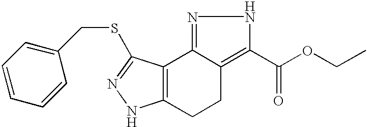 | ethyl 8-(benzylthio)-2,4,5,6- tetrahydropyrazolo[3,4- e]indazole-3-carboxylate | 200 | |
| 62 | 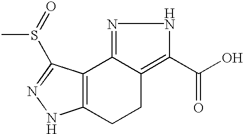 | 8-(methylsulfinyl)-2,4,5,6- tetrahydropyrazolo[3,4- e]indazole-3-carboxylic acid | 200 | |
| 63 | 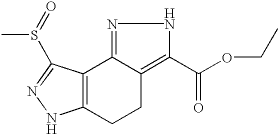 | ethyl 8-(methylsulfinyl)-2,4,5,6- tetrahydropyrazolo[3,4- e]indazole-3-carboxylate | 200 | |
| 64 | 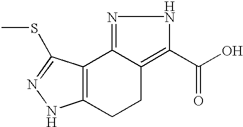 | 8-(methylthio)-2,4,5,6- tetrahydropyrazolo[3,4- e]indazole-3-carboxylic acid | 200 | |
| 65 | 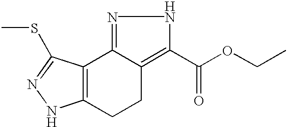 | ethyl 8-(methylthio)-2,4,5,6- tetrahydropyrazolo[3,4- e]indazole-3-carboxylate | 200 | |
| 66 | 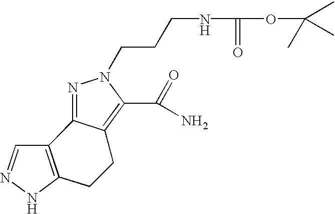 | tert-butyl 3-[3-(aminocarbonyl)- 5,6-dihydropyrazolo[3,4- e]indazol-2(4H)- yl]propylcarbamate | 200 | 200 |
| 67 | 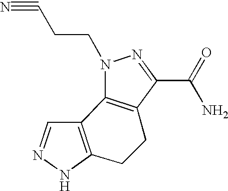 | 1-(2-cyanoethyl)-1,4,5,6- tetrahydropyrazolo[3,4- e]indazole-3-carboxamide | 200 | 200 |
| 68 | 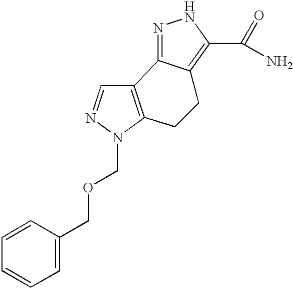 | 6-[(benzyloxy)methyl]-2,4,5,6- tetrahydropyrazolo[3,4- e]indazole-3-carboxamide | 200 | 200 |
| 69 | 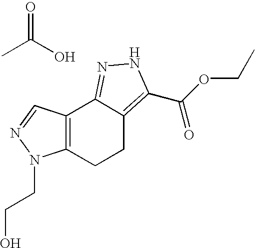 | ethyl 6-(2-hydroxyethyl)-2,4,5,6- tetrahydropyrazolo[3,4- e]indazole-3-carboxylate acetate | 200 | 200 |
| 70 | 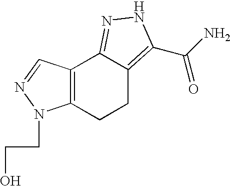 | 6-(2-hydroxyethyl)-2,4,5,6- tetrahydropyrazolo[3,4- e]indazole-3-carboxamide | 200 | 200 |
| 71 | 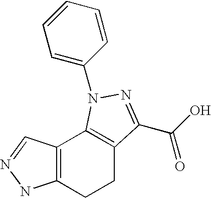 | 1-phenyl-1,4,5,6- tetrahydropyrazolo[3,4- e]indazole-3-carboxylic acid | 200 | 200 |
| 72 | 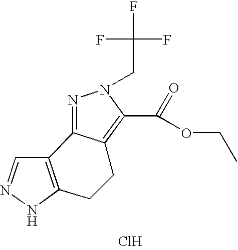 | ethyl 2-(2,2,2-trifluoroethyl)- 2,4,5,6-tetrahydropyrazolo[3,4- e]indazole-3-carboxylate hydrochloride | 200 | 200 |
| 73 | 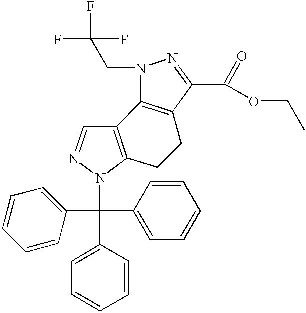 | ethyl 1-(2,2,2-trifluoroethyl)-6- trityl-1,4,5,6- tetrahydropyrazolo[3,4- e]indazole-3-carboxylate | 200 | 200 |
| 74 | 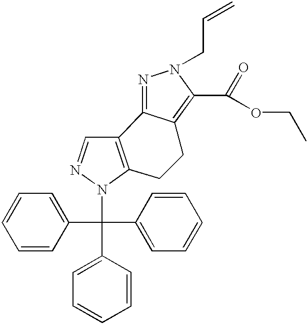 | ethyl 2-allyl-6-trityl-2,4,5,6- tetrahydropyrazolo[3,4- e]indazole-3-carboxylate | 200 | 200 |
| 75 | 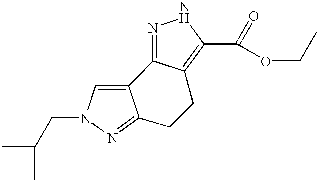 | ethyl 7-isobutyl-2,4,5,7- tetrahydropyrazolo[3,4- e]indazole-3-carboxylate | 200 | 200 |
| 76 | 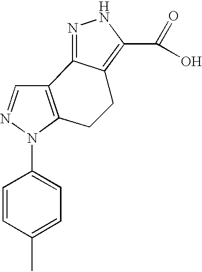 | 6-(4-methylphenyl)-2,4,5,6- tetrahydropyrazolo[3,4- e]indazole-3-carboxylic acid | 200 | 200 |
| 77 | 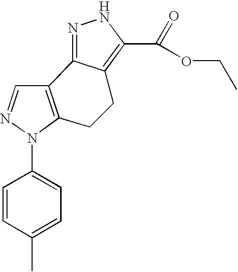 | ethyl 6-(4-methylphenyl)-2,4,5,6- tetrahydropyrazolo[3,4- e]indazole-3-carboxylate | 200 | 200 |
| 78 | 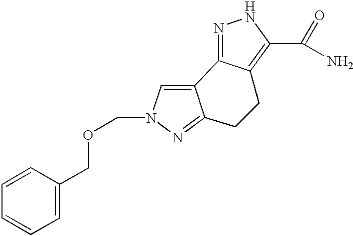 | 7-[(benzyloxy)methyl]-2,4,5,7- tetrahydropyrazolo[3,4- e]indazole-3-carboxamide | 200 | 200 |
| 79 | 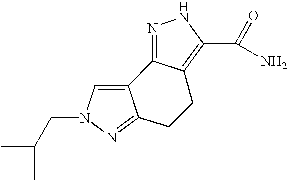 | 7-isobutyl-2,4,5,7- tetrahydropyrazolo[3,4- e]indazole-3-carboxamide | 200 | 102 |
| 80 | 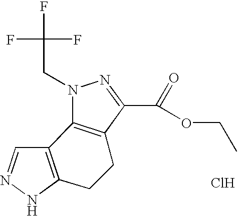 | ethyl 1-(2,2,2-trifluoroethyl)- 1,4,5,6-tetrahydropyrazolo[3,4- e]indazole-3-carboxylate hydrochloride | 200 | 124 |
| 81 | 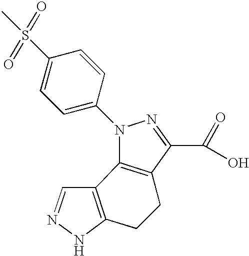 | 1-[4-(methylsulfonyl)phenyl]- 1,4,5,6- tetrahydropyrazolo[3,4- e]indazole-3-carboxylic acid | 200 | 200 |
| 82 | 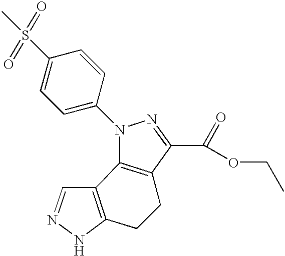 | ethyl 1-[4- (methylsulfonyl)phenyl]-1,4,5,6- tetrahydropyrazolo[3,4- e]indazole-3-carboxylate | 200 | 200 |
| 83 | 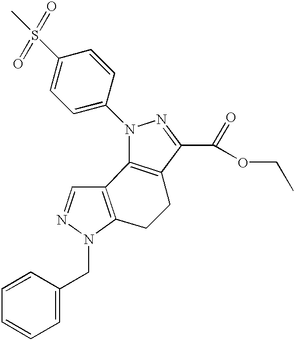 | ethyl 6-benzyl-1-[4- (methylsulfonyl)phenyl]-1,4,5,6- tetrahydropyrazolo[3,4- e]indazole-3-carboxylate | 200 | 200 |
| 84 | 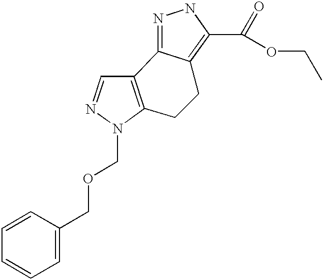 | ethyl 6-[(benzyloxy)methyl]- 2,4,5,6-tetrahydropyrazolo[3,4- e]indazole-3-carboxylate | 200 | 200 |
| 85 | 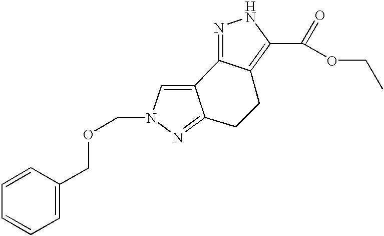 | ethyl 7-[(benzyloxy)methyl]- 2,4,5,7-tetrahydropyrazolo[3,4- e]indazole-3-carboxylate | 200 | 200 |
| 86 | 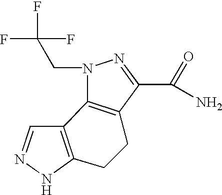 | 1-(2,2,2-trifluoroethyl)-1,4,5,6- tetrahydropyrazolo[3,4- e]indazole-3-carboxamide | 200 | 200 |
| 87 | 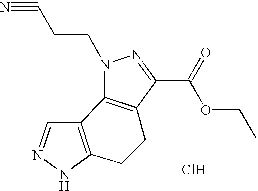 | ethyl 1-(2-cyanoethyl)-1,4,5,6- tetrahydropyrazolo[3,4- e]indazole-3-carboxylate hydrochloride | 200 | 112 |
| 88 | 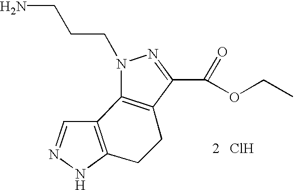 | ethyl 1-(3-aminopropyl)-1,4,5,6- tetrahydropyrazolo[3,4- e]indazole-3-carboxylate | 200 | 125 |
| 89 | 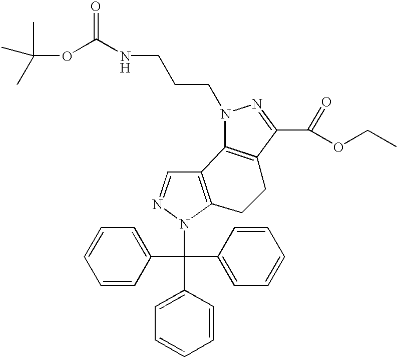 | ethyl 1-{3-[(tert- butoxycarbonyl)amino]propyl}-6- trityl-1,4,5,6- tetrahydropyrazolo[3,4- e]indazole-3-carboxylate | 200 | 200 |
| 90 | 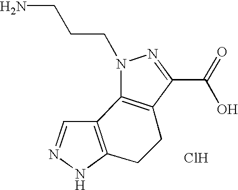 | 1-(3-aminopropyl)-1,4,5,6- tetrahydropyrazolo[3,4- e]indazole-3-carboxylic acid | 200 | 200 |
| 91 | 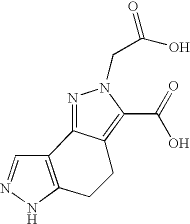 | 2-(carboxymethyl)-2,4,5,6- tetrahydropyrazolo[3,4- e]indazole-3-carboxylic acid | 200 | 200 |
| 92 | 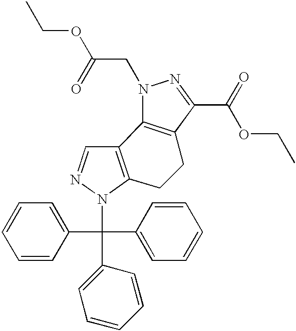 | ethyl 1-(2-ethoxy-2-oxoethyl)-6- trityl-1,4,5,6- tetrahydropyrazolo[3,4- e]indazole-3-carboxylate | 200 | 200 |
| 93 | 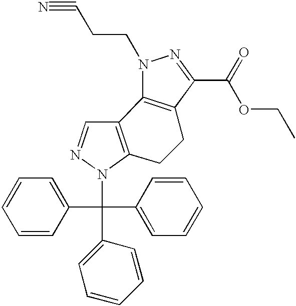 | ethyl 1-(2-cyanoethyl)-6-trityl- 1,4,5,6-tetrahydropyrazolo[3,4- e]indazole-3-carboxylate | 200 | 200 |
| 94 | 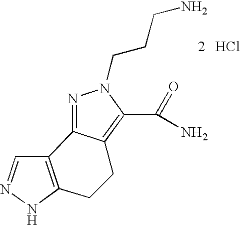 | 2-(3-aminopropyl)-2,4,5,6- tetrahydropyrazolo[3,4- e]indazole-3-carboxamide | 200 | 200 |
| 95 | 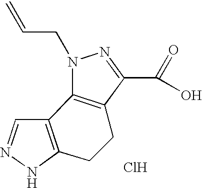 | 1-allyl-1,4,5,6- tetrahydropyrazolo[3,4- e]indazole-3-carboxylic acid hydrochloride | 200 | 200 |
| 96 | 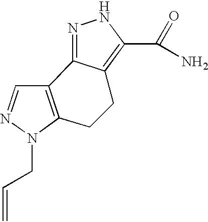 | 6-allyl-2,4,5,6- tetrahydropyrazolo[3,4- e]indazole-3-carboxamide | 200 | 200 |
| 97 | 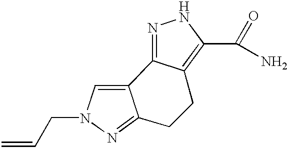 | 7-allyl-2,4,5,7- tetrahydropyrazolo[3,4- e]indazole-3-carboxamide | 200 | 169 |
| 98 | 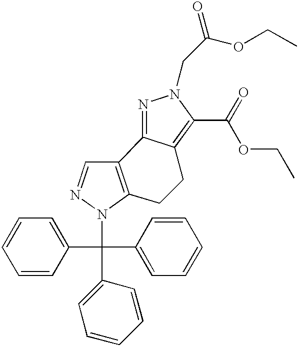 | ethyl 2-(2-ethoxy-2-oxoethyl)-6- trityl-2,4,5,6- tetrahydropyrazolo[3,4- e]indazole-3-carboxylate | 200 | 200 |
| 99 | 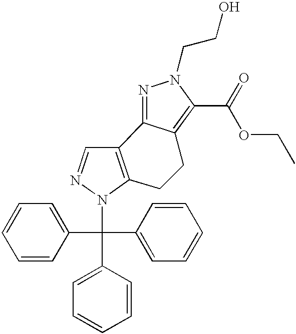 | ethyl 2-(2-hydroxyethyl)-6-trityl- 2,4,5,6-tetrahydropyrazolo[3,4- e]indazole-3-carboxylate | 200 | 200 |
| 100 | 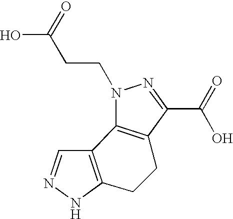 | 1-(2-carboxyethyl)-1,4,5,6-tetrahydropyrazolo[3,4- e]indazole-3-carboxylic acid | 200 | 200 |
| 101 | 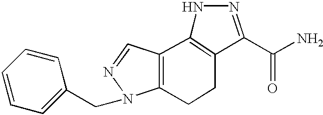 | 6-benzyl-1,4,5,6- tetrahydropyrazolo[3,4- e]indazole-3-carboxamide | 200 | 200 |
| 102 | 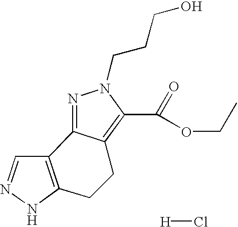 | ethyl 2-(3-hydroxypropyl)- 2,4,5,6-tetrahydropyrazolo[3,4- e]indazole-3-carboxylate hydrochloride | 200 | 200 |
| 103 | 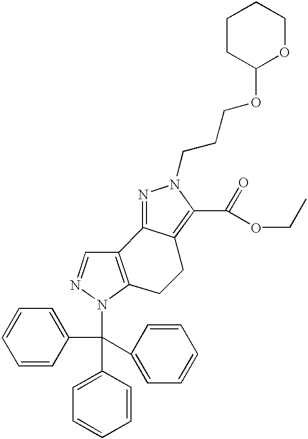 | ethyl 2-[3-(tetrahydro-2H-pyran- 2-yloxy)propyl]-6-trityl-2,4,5,6- tetrahydropyrazolo[3,4- e]indazole-3-carboxylate | 200 | 200 |
| 104 | 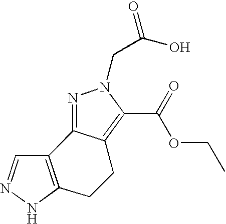 | [3-(ethoxycarbonyl)-5,6- dihydropyrazolo[3,4-e]indazol- 2(4H)-yl]acetic acid | 200 | 200 |
| 105 | 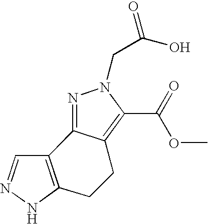 | [3-(methoxycarbonyl)-5,6- dihydropyrazolo[3,4-e]indazol- 2(4H)-yl]acetic acid | 200 | 200 |
| 106 | 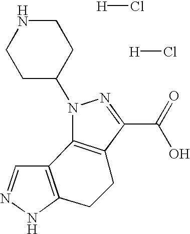 | 1-piperidin-4-yl-1,4,5,6- tetrahydropyrazolo[3,4- e]indazole-3-carboxylic acid dihydrochloride | 200 | 200 |
| 107 | 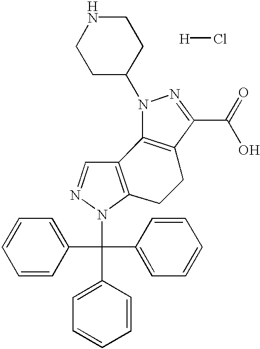 | 1-piperidin-4-yl-6-trityl-1,4,5,6- tetrahydropyrazolo[3,4- e]indazole-3-carboxylic acid hydrochloride | 200 | 200 |
| 108 | 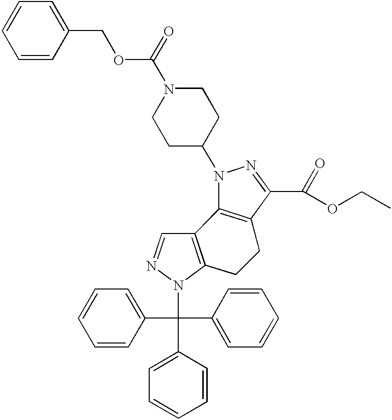 | ethyl 1-{1- [(benzyloxy)carbonyl]piperidin-4- yl}-6-trityl-1,4,5,6- tetrahydropyrazolo[3,4- e]indazole-3-carboxylate | 200 | 200 |
| 109 | 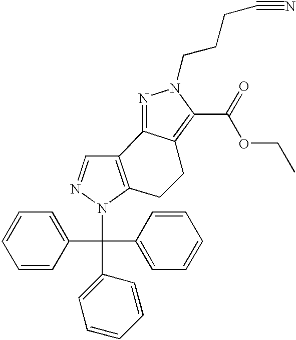 | ethyl 2-(3-cyanopropyl)-6-trityl-2,4, 5,6-tetrahydropyrazolo[3,4- e]indazole-3-carboxylate | 200 | 200 |
| 110 | 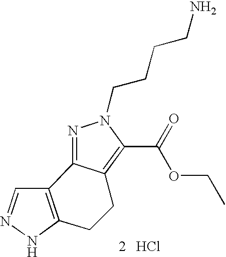 | ethyl 2-(4-aminobutyl)-2,4,5,6- tetrahydropyrazolo[3,4- e]indazole-3-carboxylate hydrochloride | 200 | 200 |
| 111 | 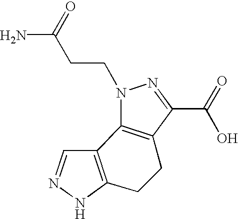 | 1-(3-amino-3-oxopropyl)-1,4,5,6- tetrahydropyrazolo[3,4- e]indazole-3-carboxylic acid | 200 | 200 |
| 112 | 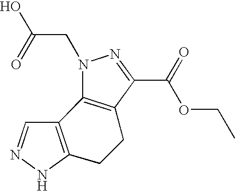 | [3-(ethoxycarbonyl)-5,6- dihydropyrazolo[3,4-e]indazol- 1(4H)-yl]acetic acid | 200 | 200 |
| 113 | 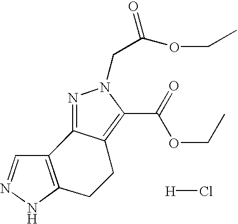 | ethyl 2-(2-ethoxy-2-oxoethyl)- 2,4,5,6-tetrahydropyrazolo[3,4- e]indazole-3-carboxylate hydrochloride | 200 | 200 |
| 114 | 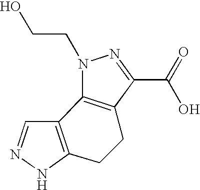 | 1-(2-hydroxyethyl)-1,4,5,6- tetrahydropyrazolo[3,4- e]indazole-3-carboxylic acid | 200 | 200 |
| 115 | 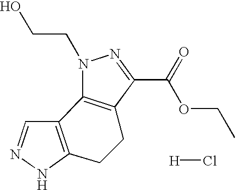 | ethyl 1-(2-hydroxyethyl)-1,4,5,6- tetrahydropyrazolo[3,4- e]indazole-3-carboxylate hydrochloride | 200 | 126 |
| 116 | 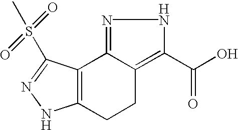 | 8-(methylsulfonyl)-2,4,5,6- tetrahydropyrazolo[3,4- e]indazole-3-carboxylic acid | 200 | |
| 117 | 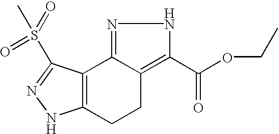 | ethyl 8-(methylsulfonyl)-2,4,5,6- tetrahydropyrazolo[3,4- e]indazole-3-carboxylate | 200 | |
| 118 | 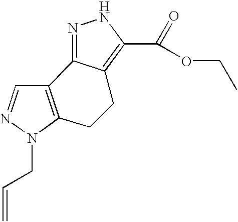 | ethyl 6-allyl-2,4,5,6- tetrahydropyrazolo[3,4- e]indazole-3-carboxylate | 200 | 200 |
| 119 | 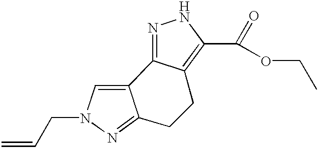 | ethyl 7-allyl-2,4,5,7- tetrahydropyrazolo[3,4- e]indazole-3-carboxylate | 200 | 200 |
| 120 | 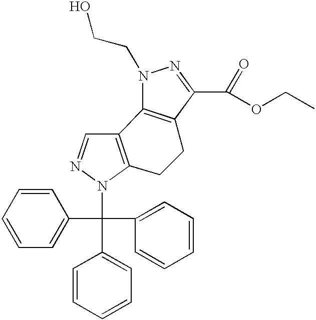 | ethyl 1-(2-hydroxyethyl)-6-trityl- 1,4,5,6-tetrahydropyrazolo[3,4- e]indazole-3-carboxylate | 200 | 200 |
| 121 | 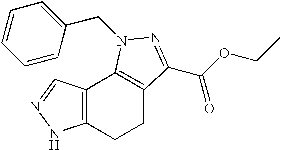 | ethyl 1-benzyl-1,4,5,6- tetrahydropyrazolo[3,4- e]indazole-3-carboxylate | 200 | 200 |
| 122 | 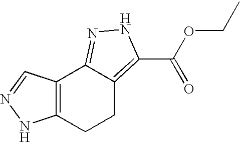 | ethyl 2,4,5,6- tetrahydropyrazolo[3,4- e]indazole-3-carboxylate | 200 | 98.4 |
| 123 | 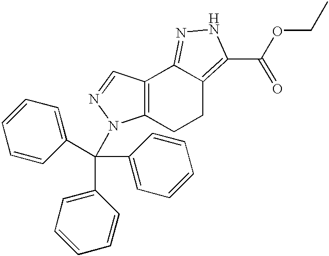 | ethyl 6-trityl-2,4,5,6- tetrahydropyrazolo[3,4- e]indazole-3-carboxylate | 200 | 200 |
| 124 | 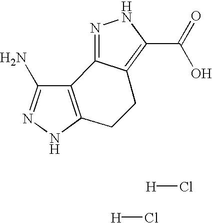 | 8-amino-2,4,5,6- tetrahydropyrazolo[3,4- e]indazole-3-carboxylic acid dihydrochloride | 200 | |
| 125 | 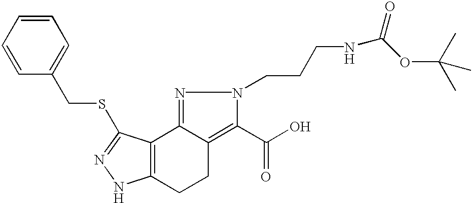 | | 200 | |
| 126 | 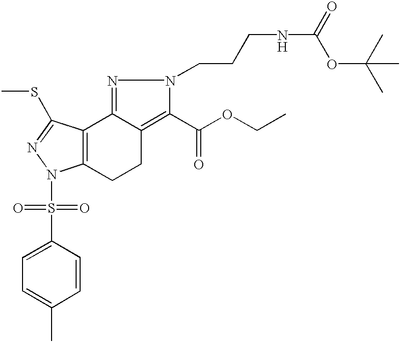 | ethyl 2-{3-[(tert- butoxycarbonyl)amino]propyl}-6- [(4-methylphenyl)sulfonyl]-8- (methylthio)-2,4,5,6- tetrahydropyrazolo[3,4- e]indazole-3-carboxylate | 200 | |
| 127 | 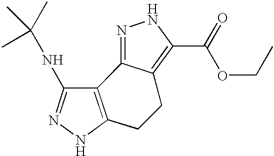 | ethyl 8-(tert-butylamino)-2,4,5,6- tetrahydropyrazolo[3,4- e]indazole-3-carboxylate | 200 | |
| 128 | 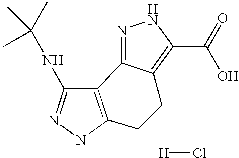 | 8-(tert-butylamino)-2,4,5,6- tetrahydropyrazolo[3,4- e]indazole-3-carboxylic acid hydrochloride | 200 | |
| 129 | 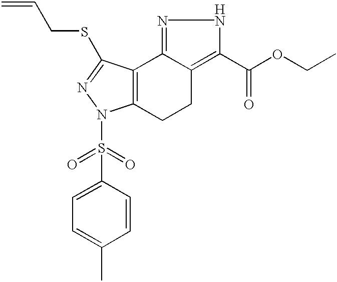 | ethyl 8-(allylthio)-6-[(4- methylphenyl)sulfonyl]-2,4,5,6- tetrahydropyrazolo[3,4- e]indazole-3-carboxylate | 200 | |
| 130 | 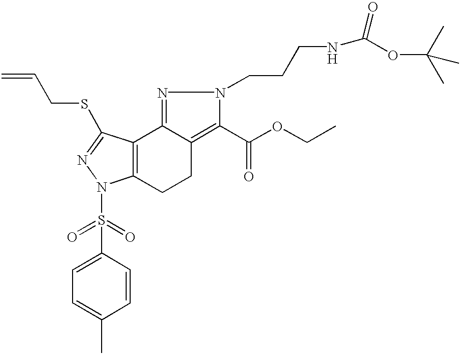 | 8-Allylsulfanyl-2-(3-tert- butoxycarbonylamino-propyl)-6- (toluene-4-sulfonyl)-2,4,5,6- tetrahydro-1,2,6,7-tetraaza-a s- indacene-3-carboxylic acid ethyl ester | 200 | |
| 131 | End MK-2 compounds | | | |
| 132 | 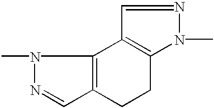 | | | |
| 133 | 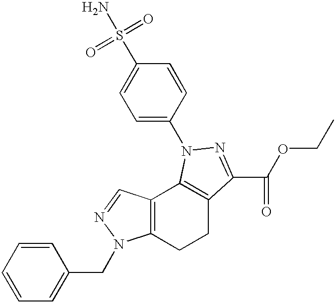 | | | |
| 134 | 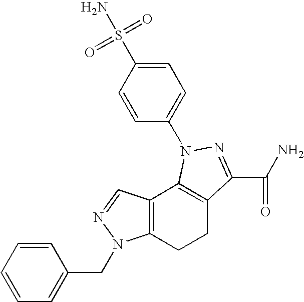 | | | |
| 135 | 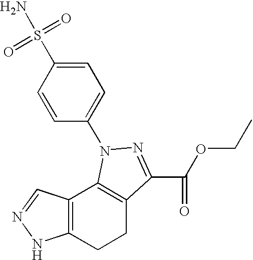 | | | |
| 136 | 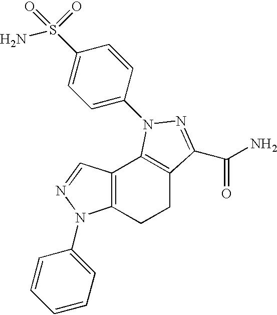 | | | |
| 137 | 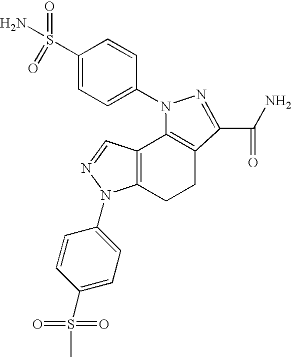 | | | |
| 138 | 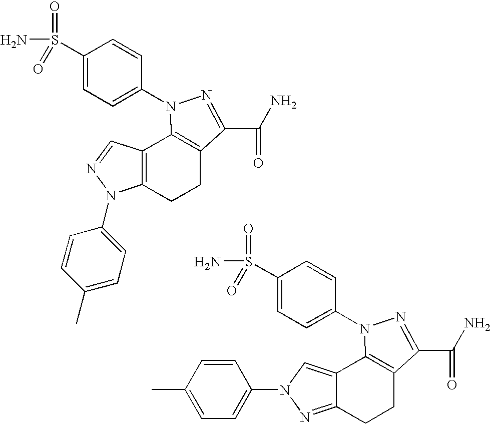 | | | |
| 139 | 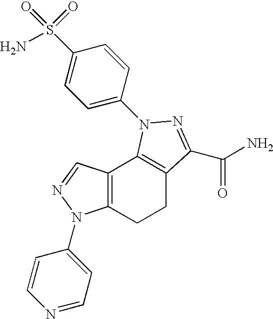 | | | |
| 140 | 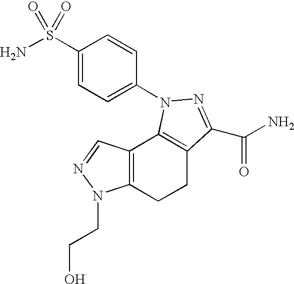 | | | |
| 141 | 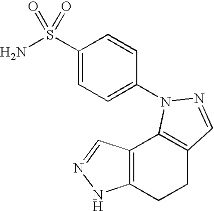 | | | |
| 142 | 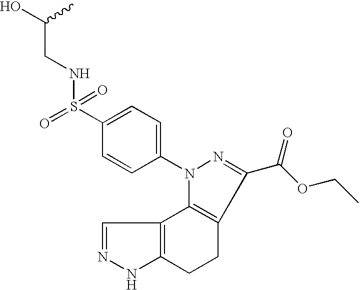 | | | |
| 143 | 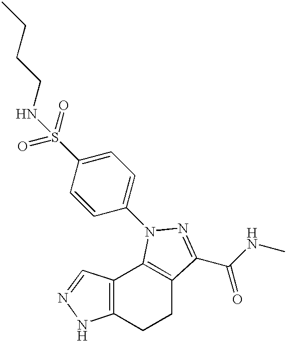 | | | |
| 144 | 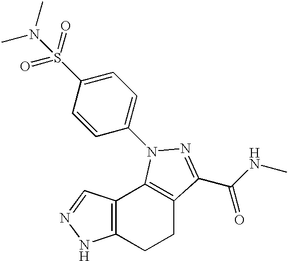 | | | |
| 145 | 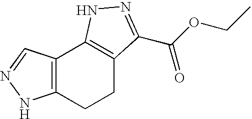 | | | |
| 146 | 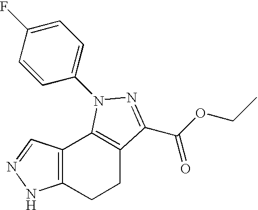 | | | |
| 147 | 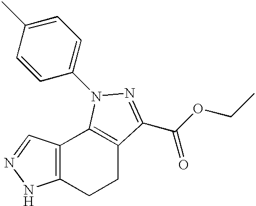 | | | |
| 148 | 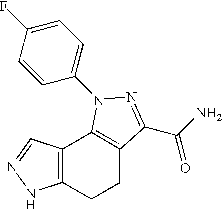 | | | |
| 149 | 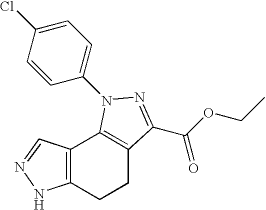 | | | |
| 150 | 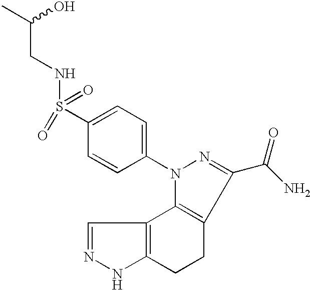 | | | |
| 151 | 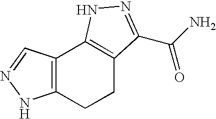 | | | |
| 152 | 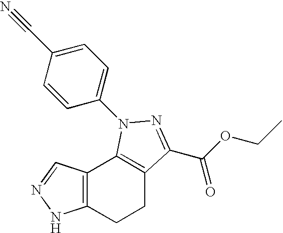 | | | |
| 153 | 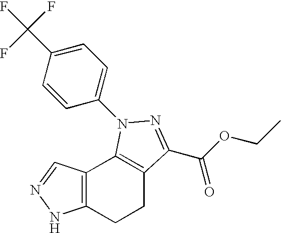 | | | |
| 154 | 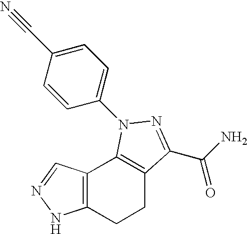 | | | |
| 155 | 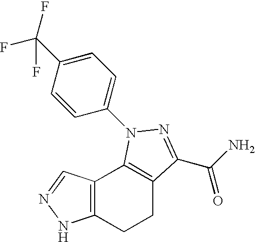 | | | |
| 156 | 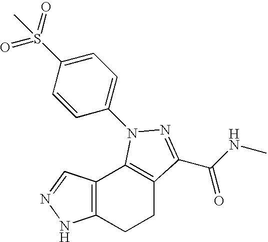 | | | |
| 157 | 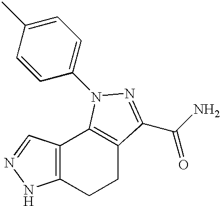 | | | |
| 158 | 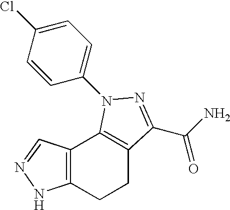 | | | |
| 159 | 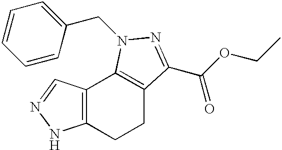 | | | |
| 160 | 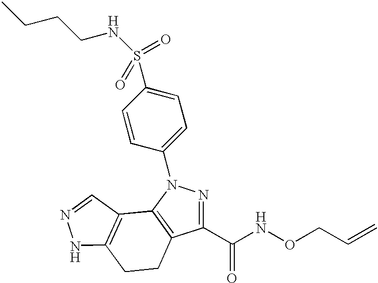 | | | |
| 161 | 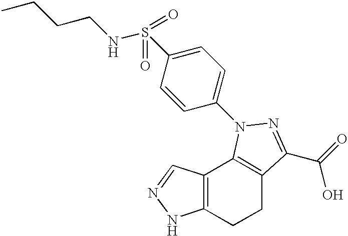 | | | |
| 162 | 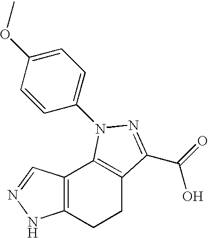 | | | |
| 163 | 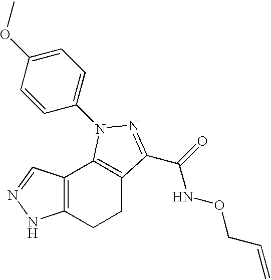 | | | |
| 164 | 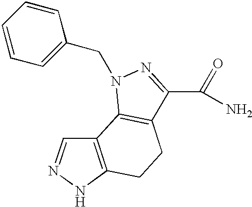 | | | |
| 165 | 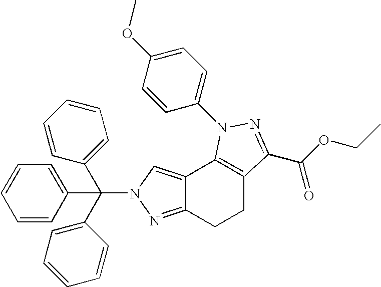 | | | |
| 166 | 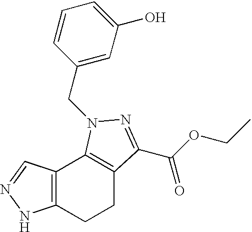 | | | |
| 167 | 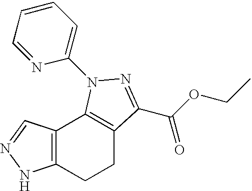 | | | |
| 168 | 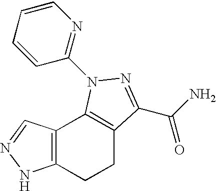 | | | |
| 169 | 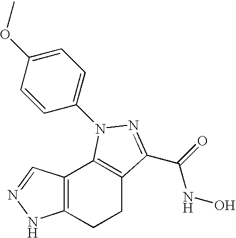 | | | |
| 170 | 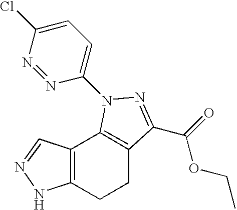 | | | |
| 171 | 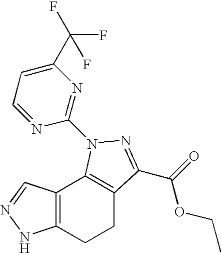 | | | |
| 172 | 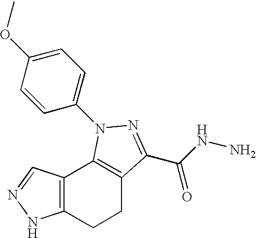 | | | |
| 173 | 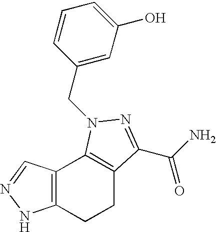 | | | |
| 174 | 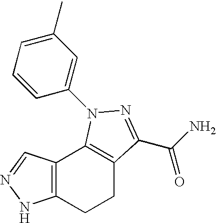 | | | |
| 175 | 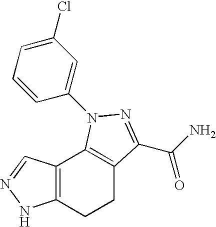 | | | |
| 176 | 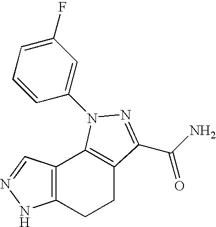 | | | |
| 177 | 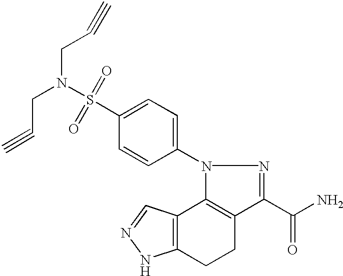 | | | |
| 178 | 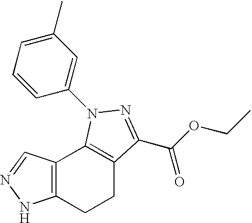 | | | |
| 179 | 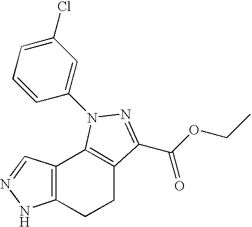 | | | |
| 180 | 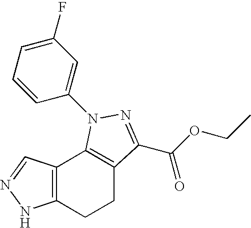 | | | |
| 181 | 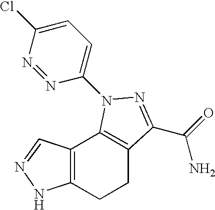 | | | |
| 182 | 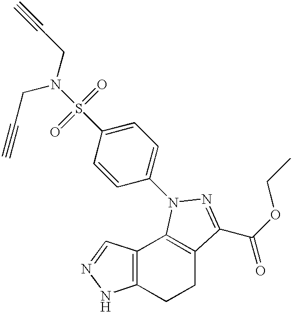 | | | |
| 183 | 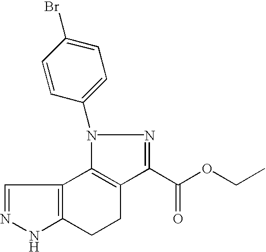 | | | |
| 184 | 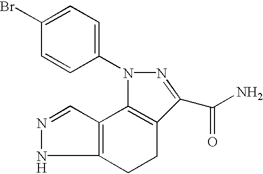 | | | |
| 185 | 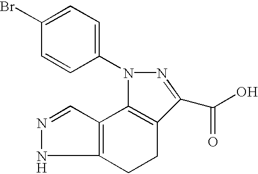 | | | |
| 186 | 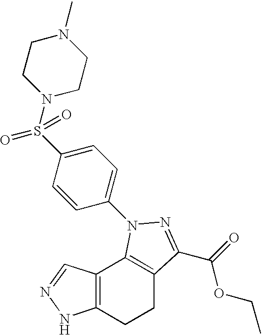 | | | |
| 187 | 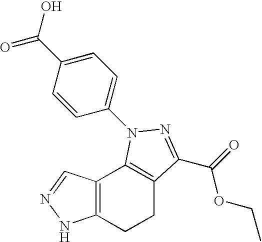 | | | |
| 188 | 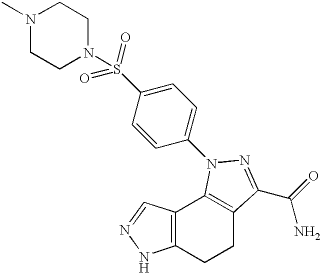 | | | |
| 189 | 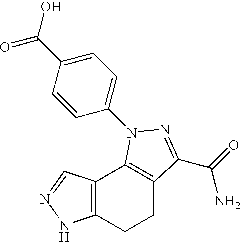 | | | |
| 190 | 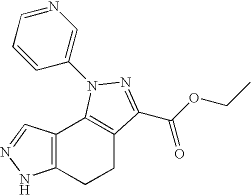 | | | |
| 191 | 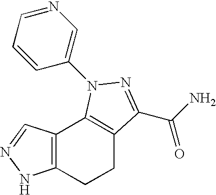 | | | |
| 192 | 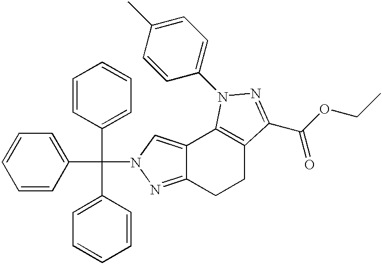 | | | |
| 193 | 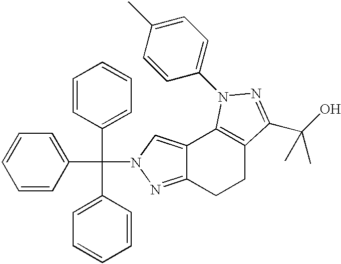 | | | |
| 194 | 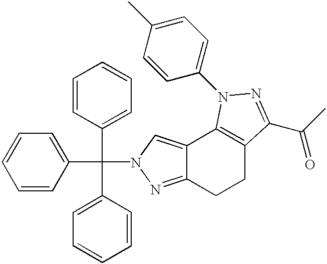 | | | |
| 195 | 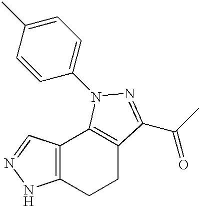 | | | |
| 196 | 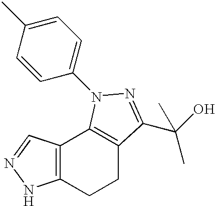 | | | |
| 197 | 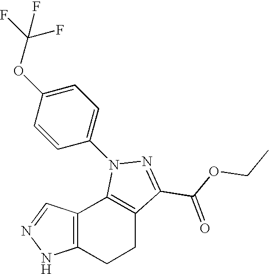 | | | |
| 198 | 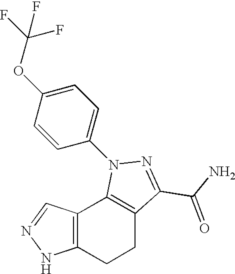 | | | |
| 199 | 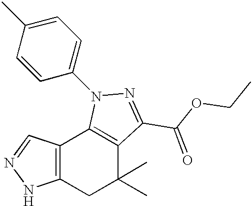 | | | |
| 200 | 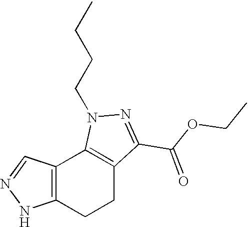 | | | |
| 201 | 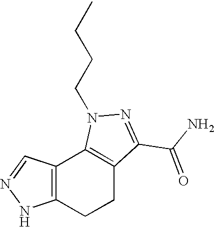 | | | |
| 202 | 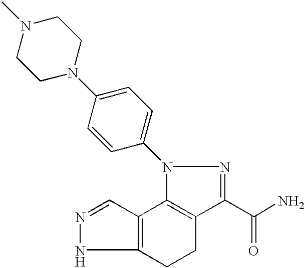 | | | |
| 203 | 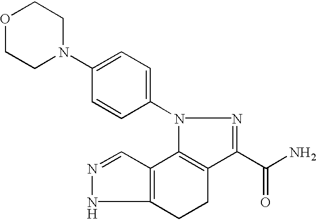 | | | |
| 204 | 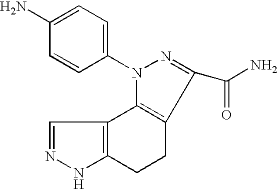 | | | |
| 205 | 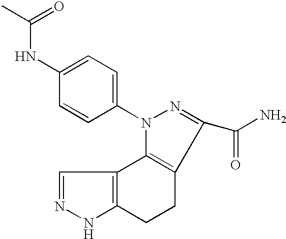 | | | |
| 206 | 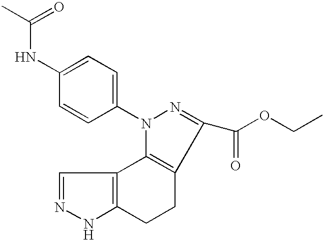 | | | |
| 207 | 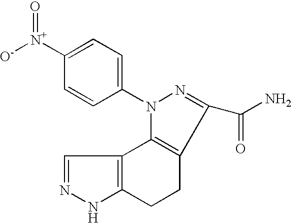 | | | |
| 208 | 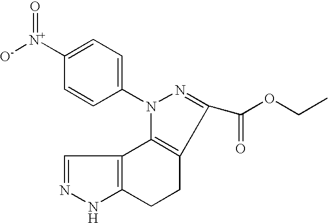 | | | |
| 209 | 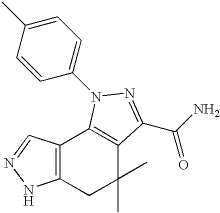 | | | |
| 210 | 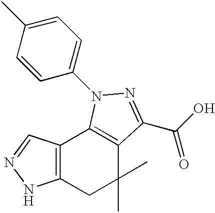 | | | |
| 211 | 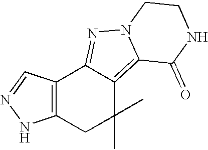 | | | |
| 212 | 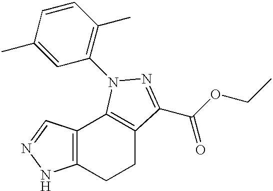 | | | |
| 213 | 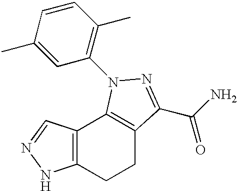 | | | |
| 214 | 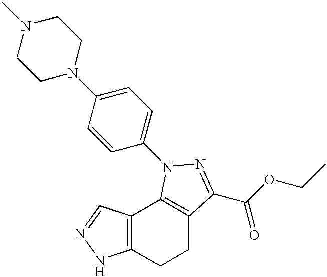 | | | |
| 215 | 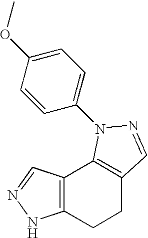 | | | |
| 216 | 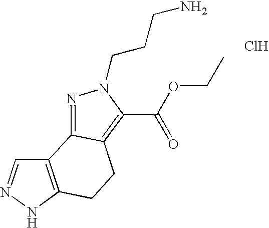 | | | |
| 217 | 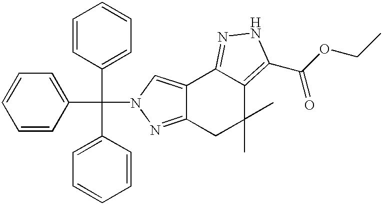 | | | |
| 218 | 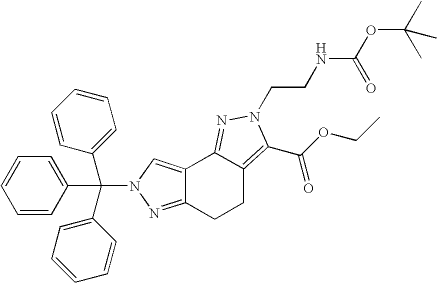 | | | |
| 219 | 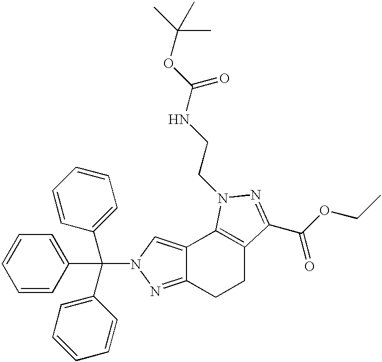 | | | |
| 220 | 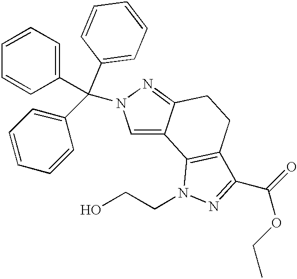 | | | |
| 221 | 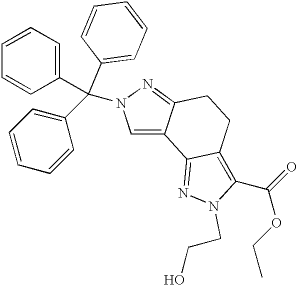 | | | |
| 222 | 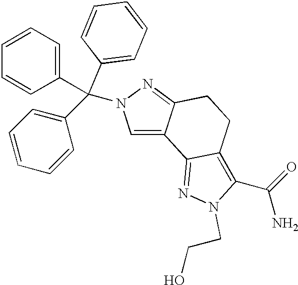 | | | |
| 223 | 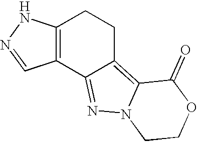 | | | |
| 224 | 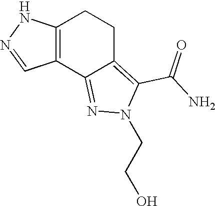 | | | |
| 225 | 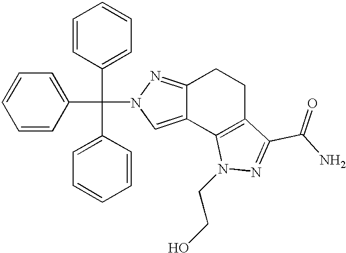 | | | |
| 226 | 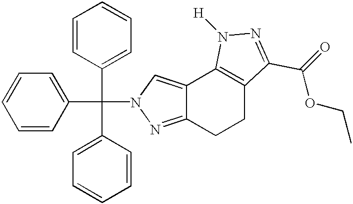 | | | |
| 227 | 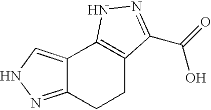 | | | |
| 228 | 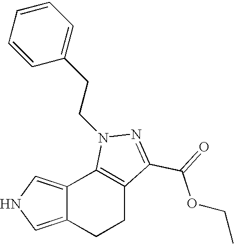 | | | |
| 229 | 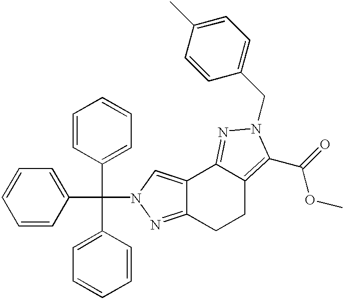 | | | |
| 230 | 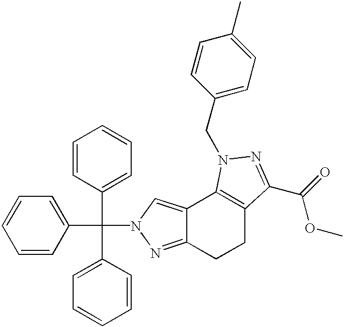 | | | |
| 231 | 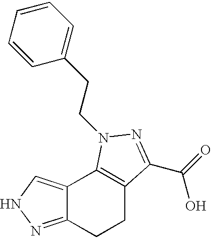 | | | |
| 232 | 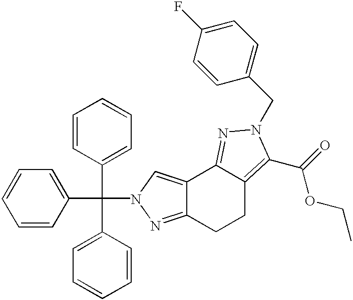 | | | |
| 233 | 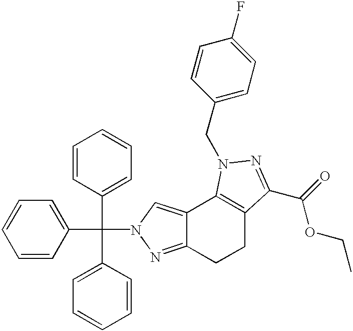 | | | |
| 234 | 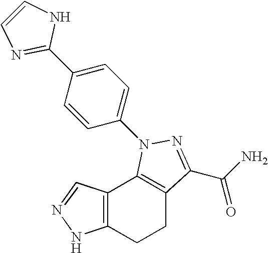 | | | |
| 235 | 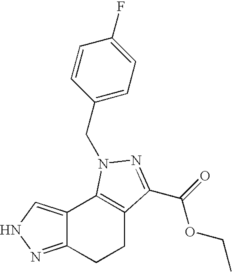 | | | |
| 236 | 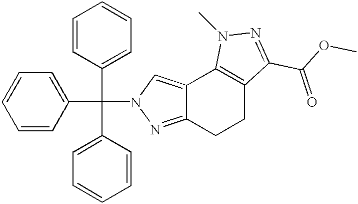 | | | |
| 237 | 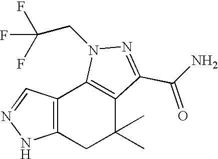 | | | |
| 238 | 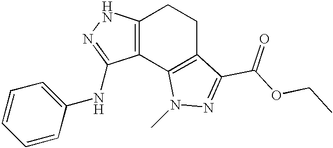 | | | |
| 239 | 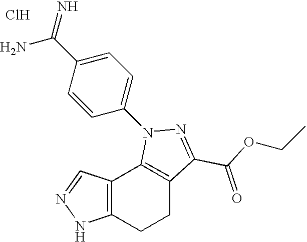 | | | |
| 240 | 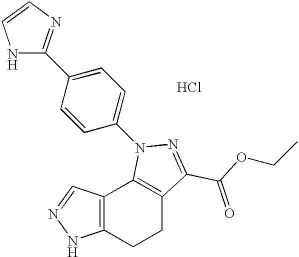 | | | |
| 241 | 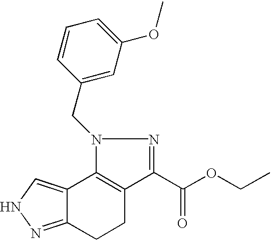 | | | |
| 242 | 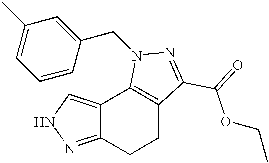 | | | |
| 243 | 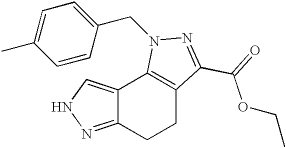 | | | |
| 244 | 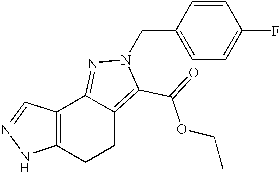 | | | |
| 245 | 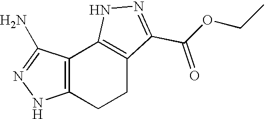 | | | |
| 246 | 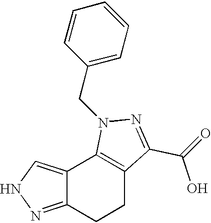 | | | |
| 247 | 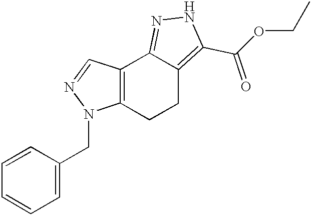 | | | |
| 248 | 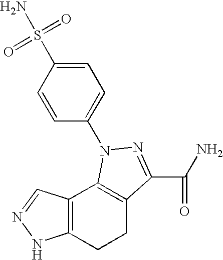 | | | |
| 249 | 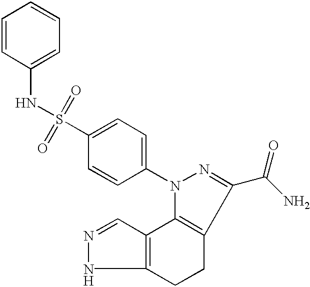 | | | |
| 250 | 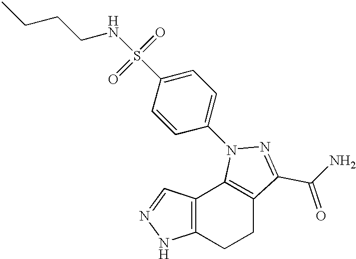 | | | |
| 251 | 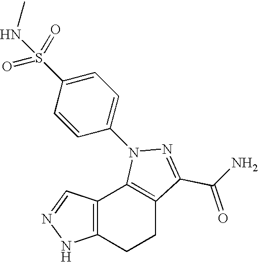 | | | |
| 252 | 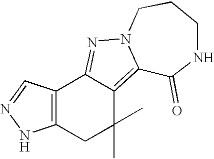 | | | |
| 253 | 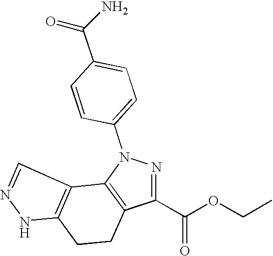 | | | |
| 254 | 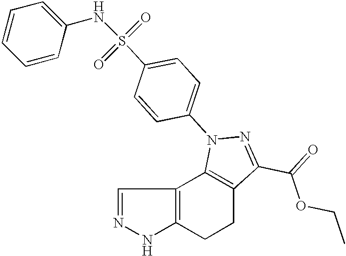 | | | |
| 255 | 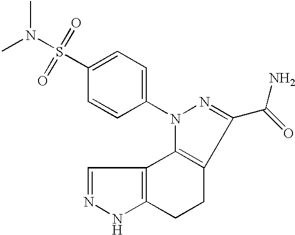 | | | |
| 256 | 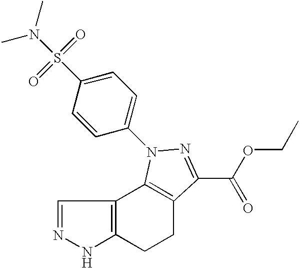 | | | |
| 257 | 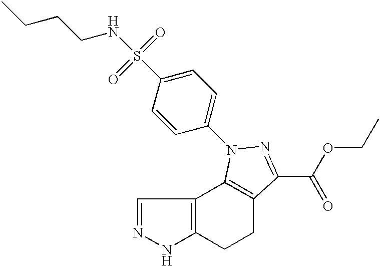 | | | |
| 258 | 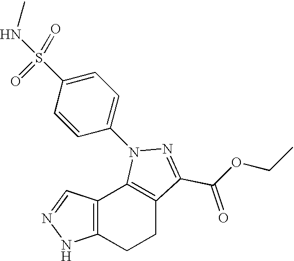 | | | |
| 259 | 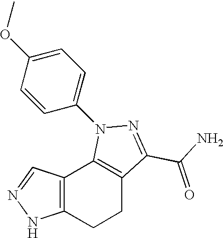 | | | |
| 260 | 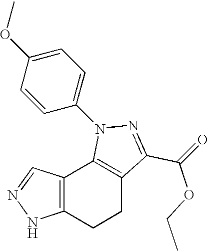 | | | |
| 261 | 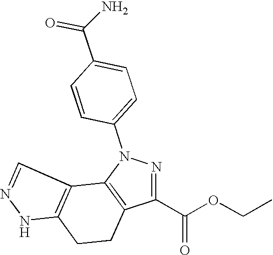 | | | |
| 262 | 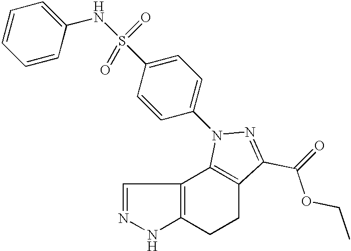 | | | |
| 263 | 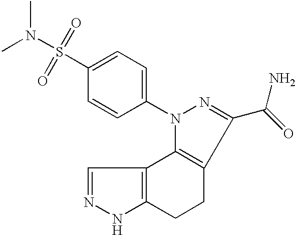 | | | |

^{a}The pyrazolylpyrazole compound may be shown with a solvent, such as, for example, trifluoroacetate, with which it can form a salt. Both the salt and acid forms of the pyrazolylpyrazole compound are included in the present invention.
^{b}Compound names generated by ACD/Name software.

TABLE 3
Pyridylpyrazole MK-2 inhibitors

| | | | MK-2 | MK-3 |
|---|---|---|---|---|
| | | | Avg. | Avg. |
| | | | IC50 | IC50 |
| No. | Structure^{a} | Chemical Name^{b} | (uM) | (uM) |
| 1 | 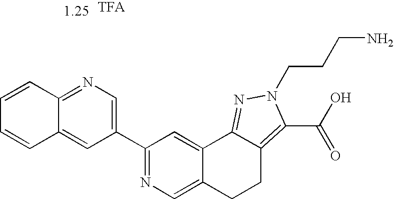 | 2-(3-amino- propyl)-8-quinoline-3-yl-4,5-di- hydro-2H-pyrazolo[3,4-f]iso- quinolin-3-carboxylic acid trifluoroacetate | 0.00946 | 0.0354 |
| 2 | 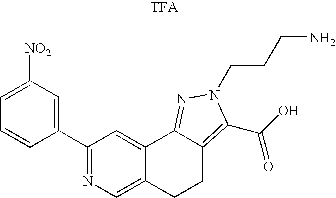 | 2-(3-amino- propyl)-8-(3-nitro- phenyl)-4,5-dihydro-2H-py- razolo[3,4-f]isoquinoline-3-carboxylic acid trifluoroacetate | 0.018 | 0.142 |
| 3 | 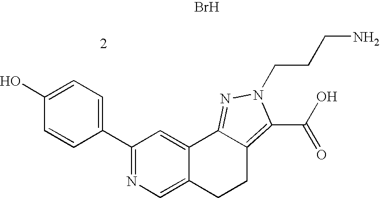 | 2-(3-aminopropyl)-8-(4-hydroxy- phenyl)-4,5-dihydro-2H-py- razolo[3,4-f]iso- quinoline-3-carboxylic acid hydrobromide | 0.0189 | 0.361 |
| 4 | 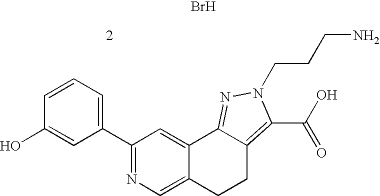 | 2-(3-aminopropyl)-8-(3-hydroxy- phenyl)-4,5-dihydro-2H-py- razolo[3,4-f]iso- quinoline-3-carboxylic acid hydrobromide | 0.0194 | 0.55 |
| 5 | 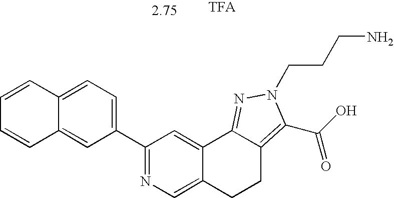 | 2-(3-aminopropyl)-8-(2-naph- thyl)-4,5-dihydro-2H-py- razolo[3,4-f]isoquino- line-3-carboxylic acid trifluoroacetate | 0.023 | 0.0698 |
| 6 | 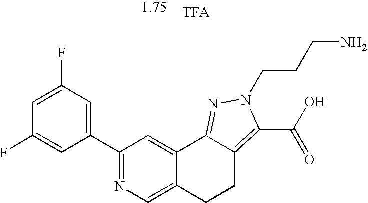 | 2-(3-aminopropyl)-8-(3,5-di- fluorophenyl)-4,5-dihydro-2H-py- razolo[3,4-f]iso- quinoline-3-carboxylic acid trifluoroacetate | 0.028 | 0.135 |
| 7 | 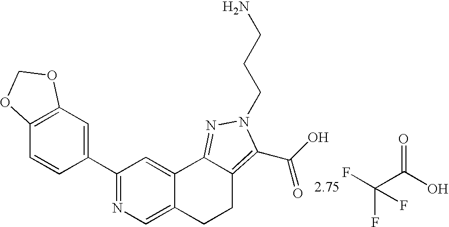 | 2-(3-aminopropyl)-8-(1,3-benzo- dioxol-5-yl)-4,5-dihydro-2H-py- razolo[3,4-f]isoquinoline-3 carboxylic acid trifluoroacetate | 0.0296 | 0.269 |
| 8 | 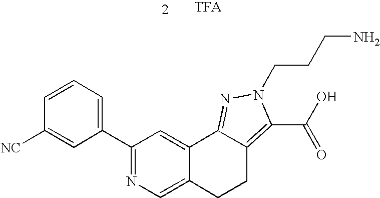 | 2-(3-aminopropyl)-8-(3-cyano- phenyl)-4,5-dihydro-2H-py- razolo[3,4-f]iso- quinoline-3-carboxylic acid trifluoroacetate | 0.0302 | 0.226 |
| 9 | 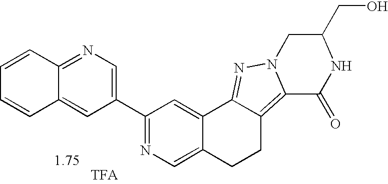 | 9-(hydroxymethyl)-2-quino- lin-3-yl-5,6,9,10-tetra- hydropyrazino[1′,2′:1,5]pyra- zolo[3,4-f]isoquinolin-7(8H)-one trifluoroacetate | 0.0313 | 5.68 |
| 10 | 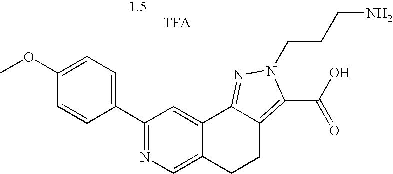 | 2-(3-aminopropyl)-8-(4-methoxy- phenyl)-4,5-dihydro-2H pyrazolo[3,4-f]iso- quinoline-3-carboxylic acid trifluoroacetate | 0.0371 | 0.995 |
| 11 | 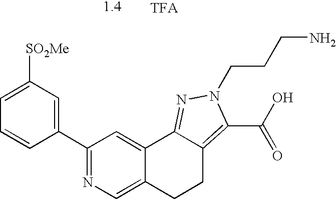 | 2-(3-aminopropyl)-8-[3-(methyl- sulfonyl)phenyl]-4,5-di- hydro-2H-pyrazolo[3,4-f]iso- quinoline-3-carboxylic acid trifluoroacetate | 0.0391 | 0.453 |
| 12 | 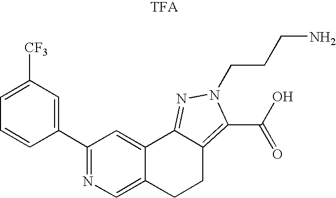 | 2-(3-aminopropyl)-8-[3-(tri- fluoromethyl)phenyl]-4,5-di- hydro-2H-pyrazolo[3,4-f]iso- quinoline-3-carboxylic acid trifluoroacetate | 0.0433 | 0.29 |
| 13 | 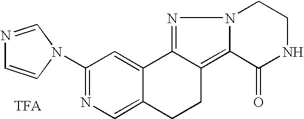 | 2-(1H-imidazol-1-yl)-5,6,9,10-tetra- hydropyrazino[1′,2′:1,5]pyra- zolo[3,4-f]isoquinolin-7(8H)-one trifluoroacetate | 0.0523 | 5.34 |
| 14 | 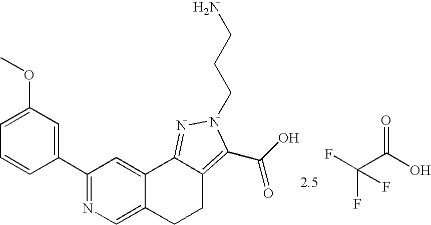 | 2-(3-aminopropyl)-8-(3-methoxy- phenyl)-4,5-dihydro-2H pyrazolo[3,4-f]iso- quinoline-3-carboxylic acid trifluoroacetate | 0.0558 | 0.875 |
| 15 | 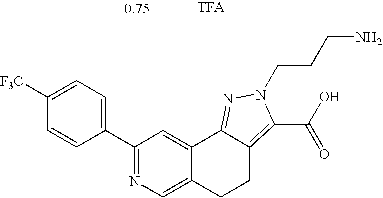 | 2-(3-aminopropyl)-8-[4-(tri- fluoromethyl)phenyl]-4,5-di- hydro-2H-pyrazolo[3,4-f]iso- quinoline-3-carboxylic acid trifluoroacetate | 0.0567 | 0.209 |
| 16 | 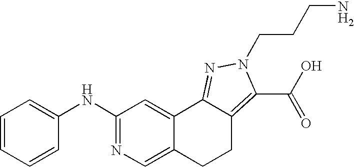 | 2-(3-aminopropyl)-8-anilino-4,5-di- hydro-2H-pyrazolo[3,4-f]iso- quinoline-3-carboxylic acid | 0.074 | 1.15 |
| 17 | 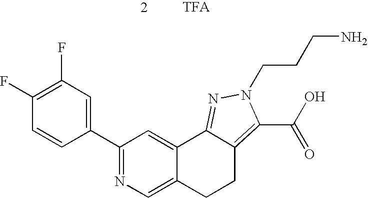 | 2-(3-aminopropyl)-8-(3,4-di- fluorophenyl)-4,5-dihydro-2H-py- razolo[3,4-f]iso- quinoline-3-carboxylic acid trifluoroacetate | 0.0786 | 1.73 |
| 18 | 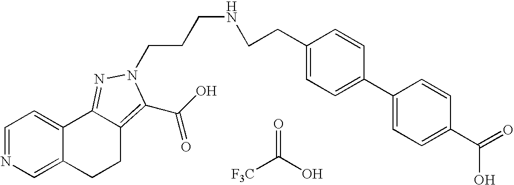 | 2-(3-{[2-(4′-carboxy-1,1′-bi- phenyl-4-yl)ethyl]a- mino}propyl)-4,5-di- hydro-2H-pyrazolo[3,4-f]iso- quinoline-3-carboxylic acid trifluoroacetate | 0.0796 | 2.18 |
| 19 | 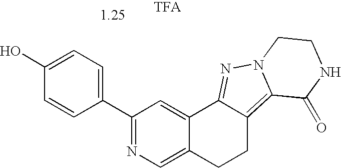 | 2-(4-hydroxyphenyl)-5,6,9,10-tetra- hydropyrazino[1′,2′:1,5]pyra- zolo[3,4-f]isoquinolin-7(8H)-one trifluoroacetate | 0.0867 | 0.861 |
| 20 | 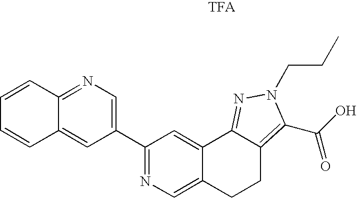 | 2-propyl-8-quinolin-3-yl-4,5-di- hydro-2H-pyrazolo[3,4-f]iso- quinoline-3-carboxylic acid | 0.0914 | |
| 21 | 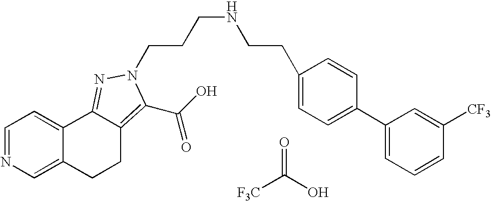 | 2-[3-({2-[3′-(trifluoromethyl)-1,1′-bi- phenyl-4-yl]ethyl}amino)pro- pyl]-4,5-dihydro-2H-py- razolo[3,4-f]iso- quinoline-3-carboxylic acid trifluoroacetate | 0.103 | 10.4 |
| 22 | 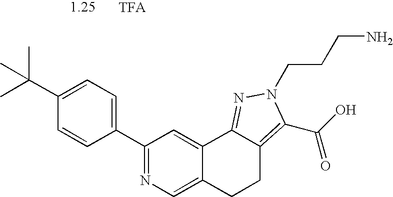 | 2-(3-aminopropyl)-8-(4-tert-butyl- phenyl)-4,5-dihydro-2H-py- razolo[3,4-f]isoquino- line-3-carboxylic acid trifluoroacetate | 0.112 | 0.426 |
| 23 | 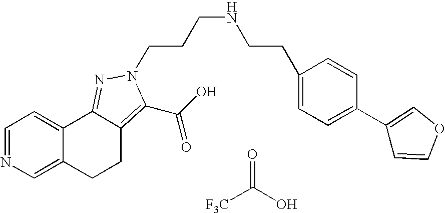 | 2-[3-({2-[4-(3-furyl)phenyl]eth- yl}amino)pro- pyl]-4,5-di- hydro-2H-pyrazolo[3,4-f]iso- quinoline-3-carboxylic acid trifluoroacetate | 0.126 | 2.62 |
| 24 | 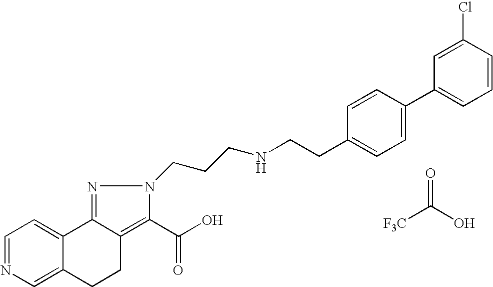 | 2-(3-{[2-(3′-chloro-1,1′-bi- phenyl-4-yl)eth- yl]amino}propyl)-4,5-di- hydro-2H-pyrazolo[3,4-f]iso- quinoline-3-carboxylic acid trifluoroacetate | 0.13 | 5.85 |
| 25 | 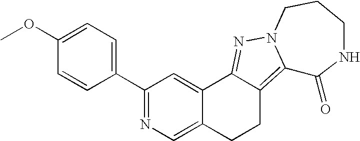 | 2-(4-methoxy- phenyl)-5,6,8,9,10,11-hexa- hydro-7H-[1,4]diaze- pino[1′,2′:1,5]pyrazolo[3,4-f]iso- quinolin-7-one | 0.142 | 6.52 |
| 26 | 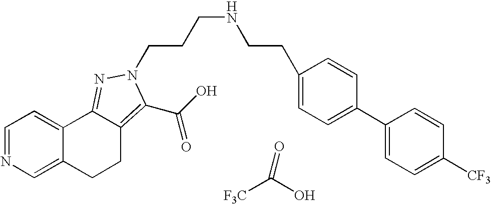 | 2-[3-({2-[4′-(trifluoromethyl)-1,1′-bi- phenyl-4-yl]eth- yl}amino)propyl]-4,5-di- hydro-2H-pyrazolo[3,4-f]iso- quinoline-3-carboxylic acid trifluoroacetate | 0.147 | 9.04 |
| 27 | 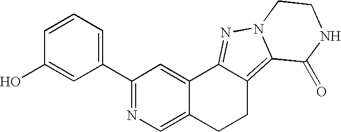 | 2-(3-hydroxyphenyl)-5,6,9,10-tetra- hydropyrazino[1′,2′:1,5]pyra- zolo[3,4-f]isoquinolin-7(8H)-one | 0.148 | 2.51 |
| 28 | 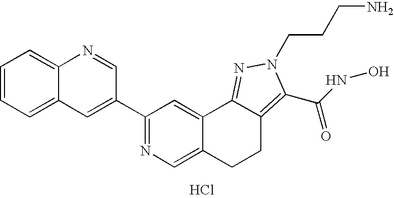 | 2-(3-amino- propyl)-N-hydroxy-8-quino- lin-3-yl-4,5-dihydro-2H-py- razolo[3,4-f]isoquinoline-3-carboxa- mide hydrochloride | 0.162 | 4.67 |
| 29 | 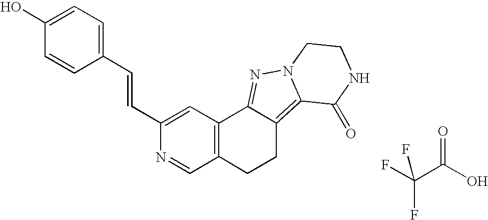 | 2-[(E)-2-(4-hydroxy- phenyl)ethenyl]-5,6,9,10 tetrahydropyrazino[1′,2′:1,5]pyra- zolo[3,4-f]isoquinolin-7(8H)-one trifluoroacetate | 0.164 | 27.5 |
| 30 | 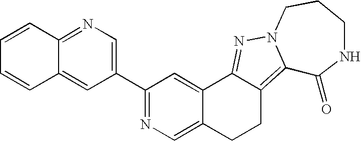 | 2-quinolin-3-yl-5,6,8,9,10,11-hexa- hydro-7H-[1,4]diaze- pino[1′,2′:1,5]pyrazolo[3,4-f]iso- quinolin-7-one | 0.171 | 11.9 |
| 31 | 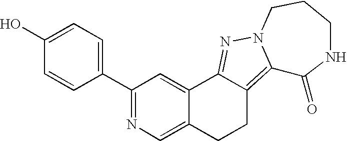 | 2-(4-hydroxy- phenyl)-5,6,8,9,10,11-hexa- hydro-7H-[1,4]diaze- pino[1′,2′:1,5]pyrazolo[3,4-f]iso- quinolin-7-one | 0.183 | 2.56 |
| 32 | 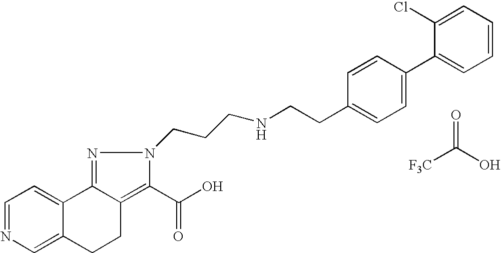 | 2-(3-{[2-(2′-chloro-1,1′-bi- phenyl-4-yl)eth- yl]amino}propyl)-4,5-di- hydro-2H-pyrazolo[3,4-f]iso- quinoline-3-carboxylic acid trifluoroacetate | 0.185 | 9.61 |
| 33 | 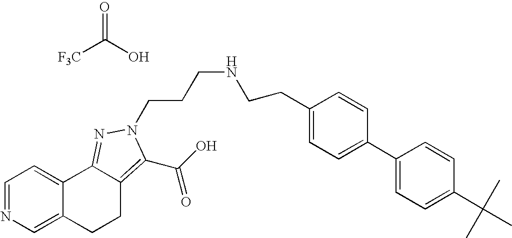 | 2-(3-{[2-(4′-tert-butyl-1,1′-bi- phenyl-4-yl)eth- yl]amino}propyl)-4,5-di- hydro-2H-pyrazolo[3,4-f]iso- quinoline-3-carboxylic acid trifluoroacetate | 0.201 | 7.22 |
| 34 | 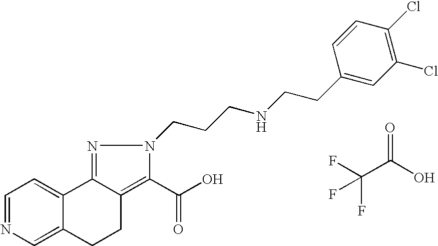 | 2-(3-{[2-(3,4-di- chlorophenyl)eth- yl]amino}propyl)-4,5-di- hydro-2H-pyrazolo[3,4-f]iso- quinoline-3-carboxylic acid trifluoroacetate | 0.201 | 10.3 |
| 35 | 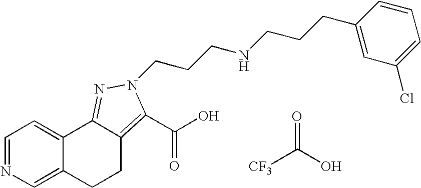 | 2-(3-{[3-(3-chloro- phenyl)propyl]amino}propyl)-4,5-di- hydro-2H-pyrazolo[3,4-f]iso- quinoline-3-carboxylic acid trifluoroacetate | 0.207 | 8.57 |
| 36 | 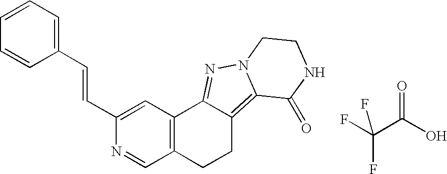 | 2-[(E)-2-phenylethenyl]-5,6,9,10 tetrahydropyrazino[1′,2′:1,5]pyra- zolo[3,4-f]isoquinolin-7(8H)-one trifluoroacetate | 0.21 | 11.5 |
| 37 | 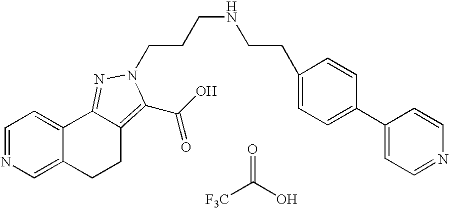 | 2-(3-{[2-(4-pyridin-4-ylphen- yl)ethyl]amino}propyl)-4,5 dihydro-2H-pyrazolo[3,4-f]iso- quinoline-3-carboxylic acid trifluoroacetate | 0.211 | 7.71 |
| 38 | 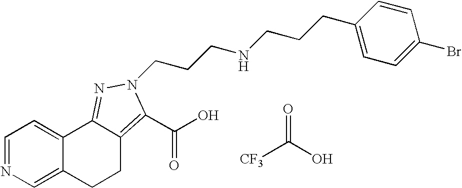 | 2-(3-{[3-(4-bromo- phenyl)propyl]amino}pro- pyl)-4,5-dihydro-2H-py- razolo[3,4-f]iso- quinoline-3-carboxylic acid trifluoroacetate | 0.22 | 10.3 |
| 39 | 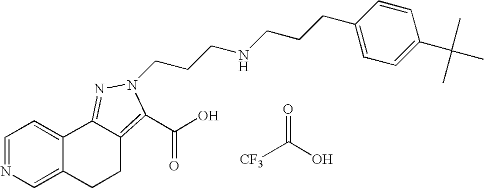 | 2-(3-{[3-(4-tert- butylphenyl)propyl]a- mino}propyl)-4,5-di- hydro-2H-pyrazolo[3,4-f]iso- quinoline-3-carboxylic acid trifluoroacetate | 0.234 | 11.6 |
| 40 | 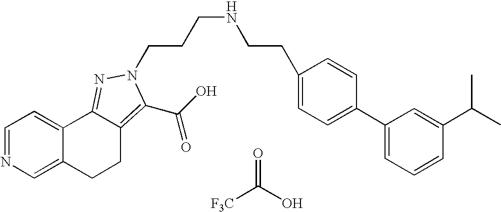 | 2-(3-{[2-(3′-isopropyl-1,1′-bi- phenyl-4-yl)ethyl]amino}pro- pyl)-4,5-dihydro-2H-pyra- zolo[3,4-f]isoquinoline-3-carboxylic acid trifluoroacetate | 0.245 | 16.1 |
| 41 | 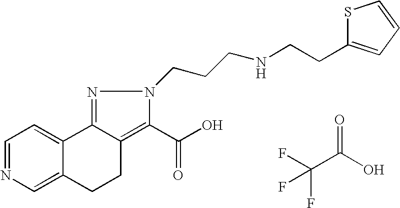 | 2-(3-[(2-thien-2-yleth- yl)amino]propyl}-4,5-di- hydro-2H-pyrazolo[3,4-f]iso- quinoline-3-carboxylic acid trifluoroacetate | 0.269 | 15.7 |
| 42 | 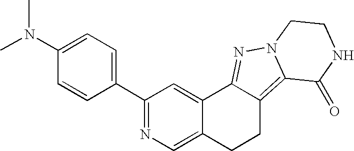 | 2-[4-(dimethyl- amino)phenyl]-5,6,9,10-tetra- hydropyrazino[1′,2′:1,5]pyra- zolo[3,4-f]isoquinolin-7(8H)-one | 0.272 | 36 |
| 43 | 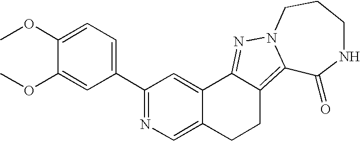 | | 0.288 | 15.8 |
| 44 | 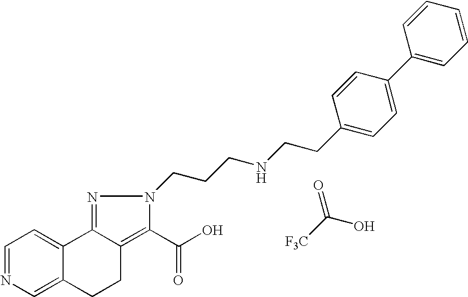 | 2-(3-{[2-(1,1′-biphenyl-4-yl)eth- yl]amino}propyl)-4,5-di- hydro-2H-pyrazolo[3,4-f]iso- quinoline-3-carboxylic acid trifluoroacetate | 0.316 | 5.01 |
| 45 | 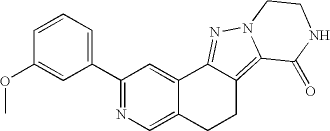 | 2-(3-methoxyphenyl)-5,6,9,10-tetra- hydropyrazino[1′,2′:1,5]pyra- zolo[3,4-f]isoquinolin-7(8H)-one | 0.322 | 200 |
| 46 | 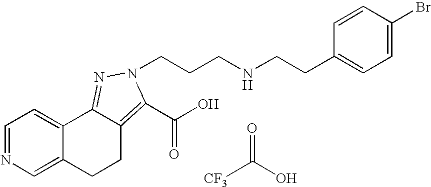 | 2-(3-{[2-(4-bromo- phenyl)ethyl]amino}propyl)-4,5-di- hydro-2H-pyrazolo[3,4-f]iso- quinoline-3-carboxylic acid trifluoroacetate | 0.325 | 10.7 |
| 47 | 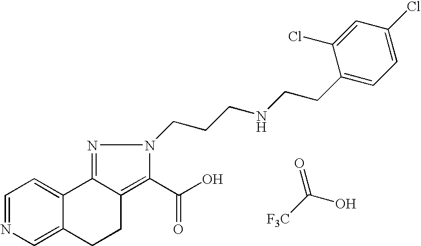 | 2-(3-{[2-(2,4-di- chlorophenyl)ethyl]amino}pro- pyl)-4,5-dihydro-2H-py- razolo[3,4-f]iso- quinoline-3-carboxylic acid trifluoroacetate | 0.347 | 6.59 |
| 48 | 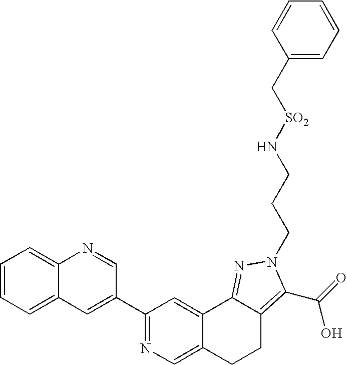 | 2-{3-[(benzyl- sulfonyl)amino]propyl}-8 quinolin-3-yl-4,5-dihydro-2H-py- razolo[3,4-f]isoquino- line-3-carboxylic acid | 0.374 | 4.51 |
| 49 | 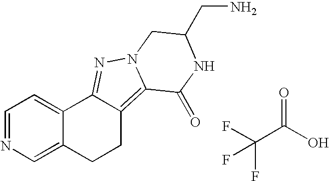 | 9-(aminomethyl)-5,6,9,10-tetra- hydropyrazino[1′,2′:1,5]pyra- zolo[3,4-f]isoquinolin-7(8H)-one trifluoroacetate | 0.427 | 45.8 |
| 50 | 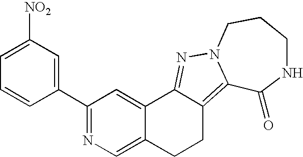 | 2-(3-nitrophenyl)-5,6,8,9,10,11-hexa- hydro-7H-[1,4]diaze- pino[1′,2′:1,5]pyrazolo[3,4-f]iso- quinolin-7-one | 0.453 | 200 |
| 51 | 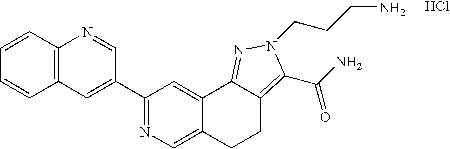 | 2-(3-amino- propyl)-8-quinolin-3-yl-4,5-di- hydro-2H-pyrazolo[3,4-f]iso- quinoline-3-carboxamide hydrochloride | 0.473 | 9.21 |
| 52 | 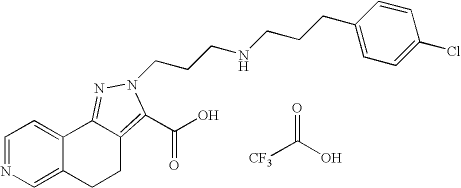 | 2-(3-{[3-(4-chloro- phenyl)propyl]amino}propyl)-4,5-di- hydro-2H-pyrazolo[3,4-f]iso- quinoline-3-carboxylic acid trifluoroacetate | 0.479 | 34.3 |
| 53 | 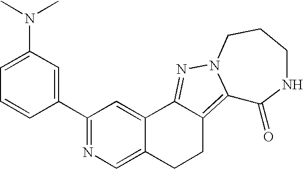 | 2-[3-(dimethyl- amino)phenyl]-5,6,8,9,10,11-hexa- hydro-7H-[1,4]diaze- pino[1′,2′:1,5]pyrazolo[3,4-f]iso- quinolin-7-one | 0.483 | 4.92 |
| 54 | 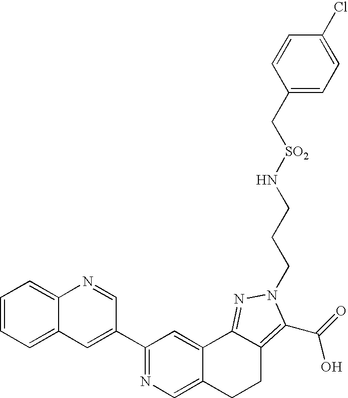 | 2-(3-{[4-chloro- benzyl)sulfonyl]amino}pro- pyl)-8-quino- lin-3-yl-4,5-dihydro-2H-pyra- zolo[3,4-f]isoquinoline-3 carboxylic acid | 0.52 | 6.46 |
| 55 | 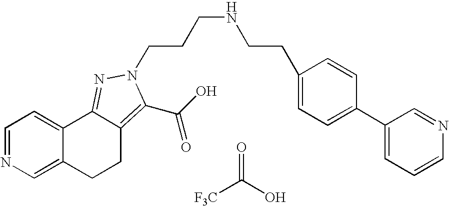 | 2-(3-{[2-(4-pyridin-3-ylphen- yl)ethyl]amino}propyl)-4,5 dihydro-2H-pyrazolo[3,4-f]iso- quinoline-3-carboxylic acid trifluoroacetate | 0.522 | 11.2 |
| 56 | 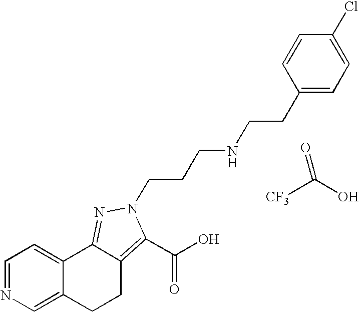 | 2-(3-{[2-(4-chloro- phenyl)ethyl]amino}propyl)-4,5-di- hydro-2H-pyrazolo[3,4-f]iso- quinoline-3-carboxylic acid trifluoroacetate | 0.551 | 7.86 |
| 57 | 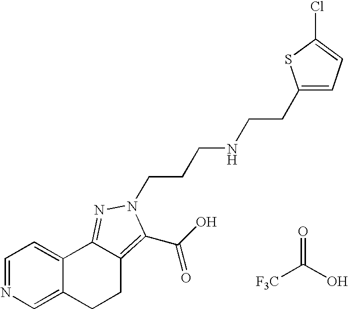 | 2-(3-{[2-(5-chlorothien-2-yl)eth- yl]amino}propyl)-4,5-di- hydro-2H-pyrazolo[3,4-f]iso- quinoline-3-carboxylic acid trifluoroacetate | 0.559 | 8.85 |
| 58 | 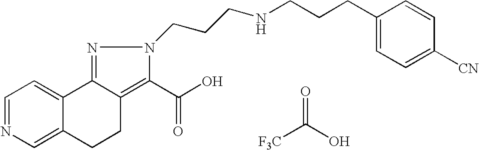 | 2-(3-{[3-(4-cyano- phenyl)propyl]amino}prop- yl)-4,5-di- hydro-2H-pyrazolo[3,4-f]iso- quinoline-3-carboxylic acid trifluoroacetate | 0.561 | 44.3 |
| 59 | 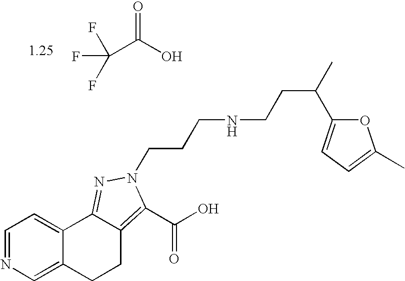 | 2-(3-{[3-(5-methyl-2-furyl)bu- tyl]amino}propyl)-4,5-di- hydro-2H-pyrazolo[3,4-f]iso- quinoline-3-carboxylic acid trifluoroacetate | 0.574 | 22.5 |
| 60 | 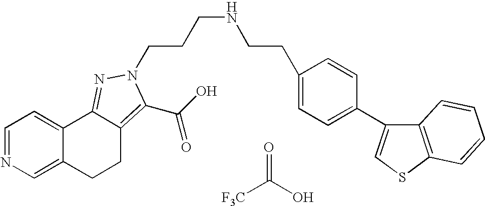 | 2-[3-({2-[4-(1-benzothien-3-yl)phen- yl]ethyl}amino)propyl]-4,5-di- hydro-2H-pyrazolo[3,4-f]iso- quinoline-3-carboxylic acid trifluoroacetate | 0.575 | 15.6 |
| 61 | 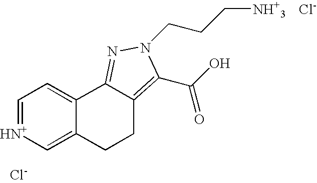 | 2-(3-ammoniopropyl)-3-carboxy 4,5-dihydro-2H-pyrazolo[3,4-f]iso- quinolin-7-ium dichloride | 0.581 | 42.6 |
| 62 | 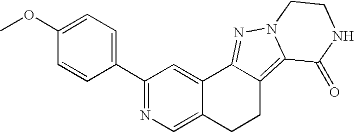 | 2-(4-methoxyphenyl)-5,6,9,10-tetra- hydropyrazino[1′,2′:1,5]pyra- zolo[3,4-f]isoquinolin-7(8H)-one | 0.587 | 200 |
| 63 | 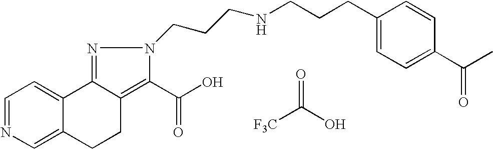 | 2-(3-{[3-(4-acetyl- phenyl)propyl]amino}prop- yl)-4,5-dihydro-2H-pyra- zolo[3,4-f]iso- quinoline-3-carboxylic acid trifluoroacetate | 0.603 | 12.6 |
| 64 | 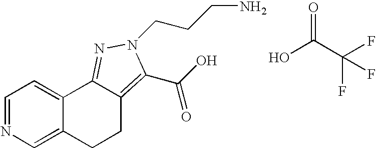 | 2-(3-amino- propyl)-4,5-dihydro-2H-py- razolo[3,4-f]isoquinoline-3 carboxylic acid trifluoroacetate | 0.62 | 22.8 |
| 65 | 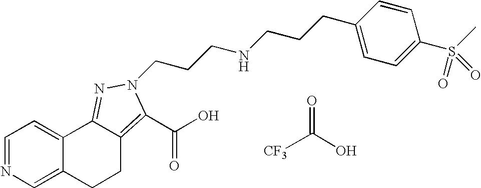 | 2-[3-({3-[4-(methyl- sulfonyl)phenyl]propyl}a- mino)propyl]-4,5-dihydro-2H-py- razolo[3,4-f]iso- quinoline-3-carboxylic acid trifluoroacetate | 0.622 | 26.1 |
| 66 | 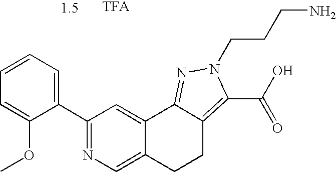 | 2-(3-aminopropyl)-8-(2-methoxy- phenyl)-4,5-dihydro-2H pyrazolo[3,4-f]iso- quinoline-3-carboxylic acid trifluoroacetate | 0.632 | 6.87 |
| 67 | 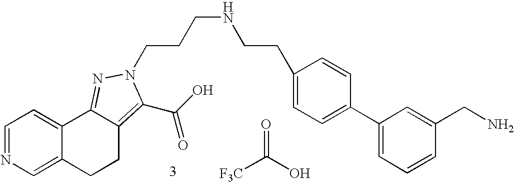 | 2-[3-{(2-[3′-(aminomethyl)-1,1′-bi- phenyl-4-yl]ethyl}amino)pro- pyl]-4,5-dihydro-2H-pyra- zolo[3,4-f]iso- quinoline-3-carboxylic acid trifluoroacetate | 0.663 | 10 |
| 68 | 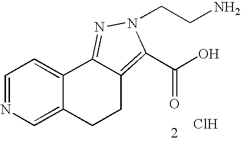 | 2-(2-aminoethyl)-4,5-dihydro-2H pyrazolo[3,4-f]iso- quinoline-3-carboxylic acid dihydrochloride | 0.706 | 43.1 |
| 69 | 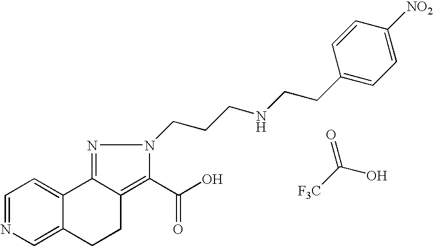 | 2-(3-{[2-(4-nitro- phenyl)ethyl]amino}propyl)-4,5-di- hydro-2H-pyrazolo[3,4-f]iso- quinoline-3-carboxylic acid trifluoroacetate | 0.765 | 11.9 |
| 70 | 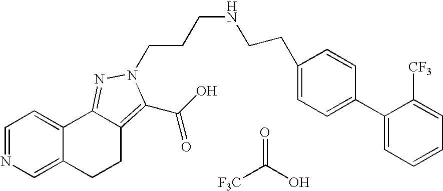 | 2-[3-({2-[2′-(trifluoromethyl)-1,1′-bi- phenyl-4-yl]eth- yl}amino)propyl]-4,5-di- hydro-2H-pyrazolo[3,4-f]iso- quinoline-3-carboxylic acid trifluoroacetate | 0.801 | 44.8 |
| 71 | 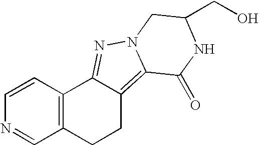 | 9-(hydroxymethyl)-5,6,9,10-tetra- hydropyrazino[1′,2′:1,5]pyra- zolo[3,4-f]isoquinolin-7(8H)-one | 0.825 | 35 |
| 72 | 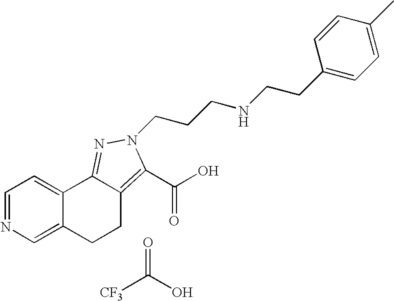 | 2-(3-{[2-(4-methyl- phenyl)ethyl]amino}propyl)-4,5-di- hydro-2H-pyrazolo[3,4-f]iso- quinoline-3-carboxylic acid trifluoroacetate | 0.89 | 17.2 |
| 73 | 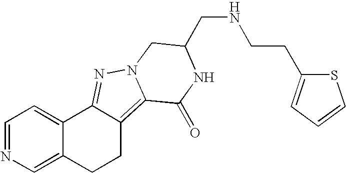 | 9-{[(2-thien-2-yleth- yl)amino]methyl}-5,6,9,10-tetra- hydropyrazino[1′,2′:1,5]pyra- zolo[3,4-f]isoquinolin-7(8H)-one | 0.924 | 27.8 |
| 74 | 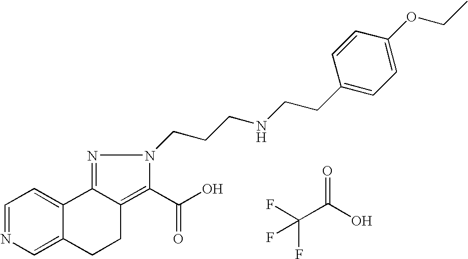 | 2-(3-{[2-(4-ethoxy- phenyl)ethyl]amino}propyl)-4,5-di- hydro-2H-pyrazolo[3,4-f]iso- quinoline-3-carboxylic acid trifluoroacetate | 0.931 | 34.8 |
| 75 | 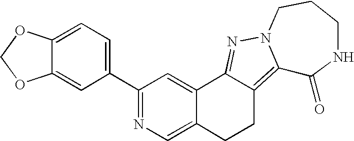 | 2-(1,3-benzo- dioxol-5-yl)-5,6,8,9,10,11-hexa- hydro-7H-[1,4]diaze- pino[1′,2′:1,5]pyrazolo[3,4-f]iso- quinolin-7-one | 0.935 | 200 |
| 76 | 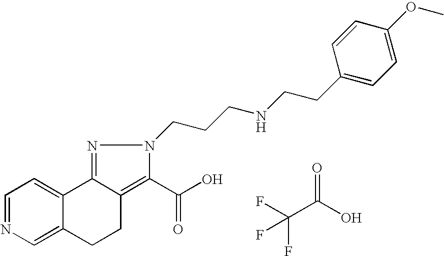 | 2-(3-{[2-(4-methoxy- phenyl)ethyl]amino}pro- pyl)-4,5-dihydro-2H-pyra- zolo[3,4-f]isoquinoline-3-carboxylic acid trifluoroacetate | 0.939 | 16.1 |
| 77 | 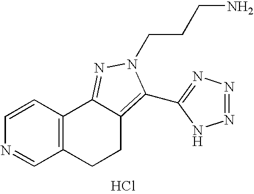 | 3-[3-(1H-tetraazol-5-yl)-4,5-di- hydro-2H-pyrazolo[3,4-f]iso- quinolin-2-yl]propan-1-amine hydrochloride | 1.08 | 72.4 |
| 78 | 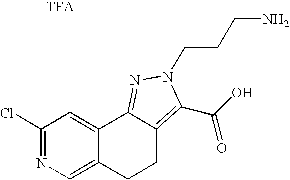 | 2-(3-aminopropyl)-8-chloro-4,5-di- hydro-2H-pyrazolo[3,4-f]iso- quinoline-3-carboxylic acid trifluoroacetate | 1.1 | 55.7 |
| 79 | 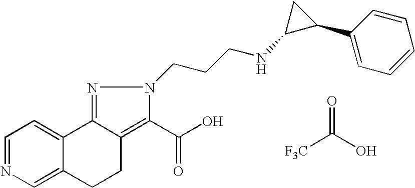 | 2-(3-{[(1R,2S)-2-phenyl- cyclopropyl]amino}propyl)-4,5-di- hydro-2H-pyrazolo[3,4-f]iso- quinoline-3-carboxylic acid trifluoroacetate | 1.15 | 47.8 |
| 80 | 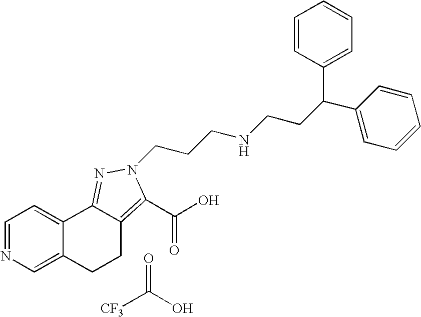 | 2-{3-[(3,3-di- phenylpropyl)amino]propyl}-4,5-di- hydro-2H-pyrazolo[3,4-f]iso- quinoline-3-carboxylic acid trifluoroacetate | 1.18 | 30.3 |
| 81 | 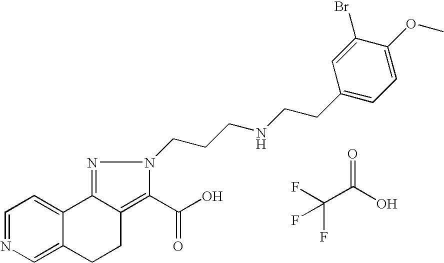 | 2-(3-{[2-(3-bromo-4-methoxy- phenyl)ethyl]amino}pro- pyl)-4,5-dihydro-2H-pyra- zolo[3,4-f]isoquinoline-3-carboxylic acid trifluoroacetate | 1.22 | 33 |
| 82 | 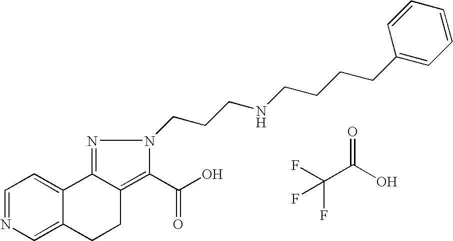 | 2-{3-[(4-phenyl- butyl)amino]propyl}-4,5-di- hydro-2H-pyrazolo[3,4-f]iso- quinoline-3-carboxylic acid trifluoroacetate | 1.26 | 40.6 |
| 83 | 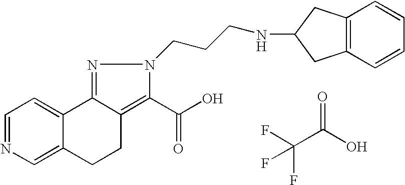 | 2-[3-(2,3-dihydro-1H-inden-2-yla- mino)propyl]-4,5-dihydro-2H-pyra- zolo[3,4-f]isoquinoline-3-carboxylic acid trifluoroacetate | 1.26 | 31.1 |
| 84 | 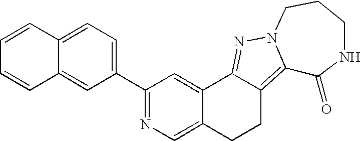 | 2-(2-naphthyl)-5,6,8,9,10,11-hexa- hydro-7H-[1,4]diaze- pino[1′,2′:1,5]pyrazolo[3,4-f]iso- quinolin-7-one | 1.3 | 2.13 |
| 85 | 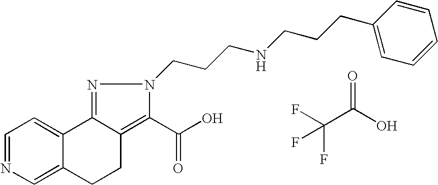 | 2-{3-[(3-phenyl- propyl)amino]propyl}-4,5-di- hydro-2H-pyrazolo[3,4-f]iso- quinoline-3-carboxylic acid trifluoroacetate | 1.33 | 200 |
| 86 | 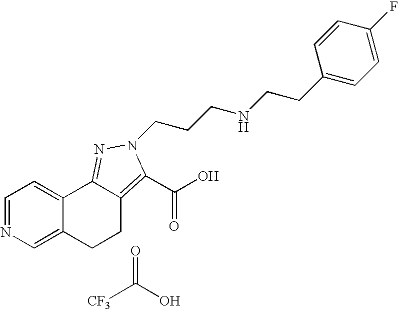 | 2-(3-{[2-(4-fluoro- phenyl)ethyl]amino}propyl)4,5-di- hydro-2H-pyrazolo[3,4-f]iso- quinoline-3-carboxylic acid trifluoroacetate | 1.34 | 20.2 |
| 87 | 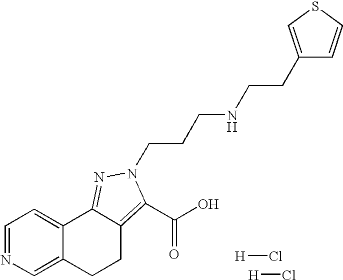 | 2-{3-[(2-thien-3-yleth- yl)amino]propyl}-4,5-di- hydro-2H-pyrazolo[3,4-f]iso- quinoline-3-carboxylic acid dihydrochloride | 1.38 | 34.1 |
| 88 | 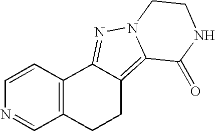 | 5,6,9,10-tetra- hydropyrazino[1′,2′:1,5]pyra- zolo[3,4-f]isoquinolin-7(8H)-one | 1.45 | 103 |
| 89 | 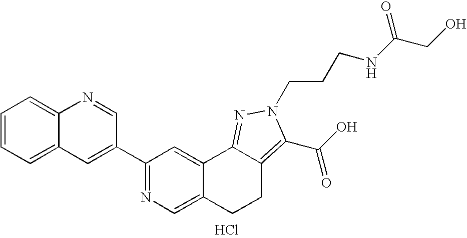 | 2-[3-(glycoloyl- amino)propyl]-8-quino- lin-3-yl-4,5-dihydro-2H-pyra- zolo[3,4-f]isoquinoline-3-carboxylic acid hydrochloride | 1.48 | 11.3 |
| 90 | 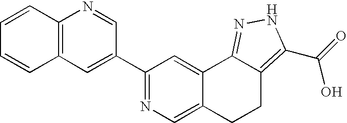 | 8-quinolin-3-yl-4,5-dihydro-2H-pyra- zolo[3,4-f]isoquinoline-3-carboxylic acid | 1.51 | 11.3 |
| 91 | 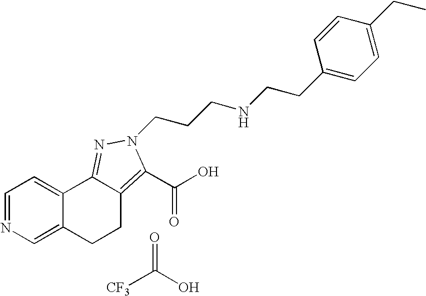 | 2-(3-{[2-(4-ethyl- phenyl)ethyl]amino}propyl)-4,5-di- hydro-2H-pyrazolo[3,4-f]iso- quinoline-3-carboxylic acid trifluoroacetate | 1.52 | 44.2 |
| 92 | 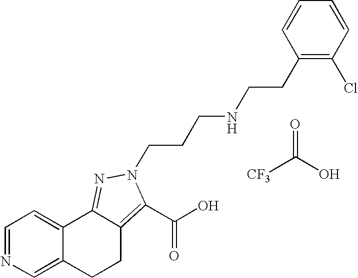 | 2-(3-{[2-(2-chloro- phenyl)ethyl]amino}propyl)-4,5-di- hydro-2H-pyrazolo[3,4-f]iso- quinoline-3-carboxylic acid trifluoroacetate | 1.53 | 49.3 |
| 93 | 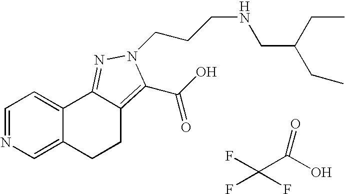 | 2-{3-[(2-ethyl- butyl)amino]propyl}4,5-di- hydro-2H-pyrazolo[3,4-f]iso- quinoline-3-carboxylic acid trifluoroacetate | 1.55 | 44.1 |
| 94 | 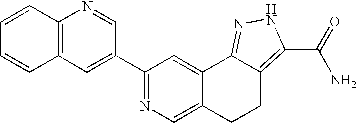 | 8-quinolin-3-yl-4,5-dihydro-2H-pyra- zolo[3,4-f]iso- quinoline-3-carboxamide | 1.63 | 200 |
| 95 | 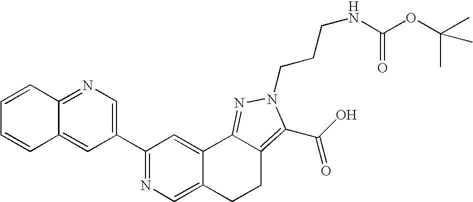 | PHA-00767498 | 1.64 | 17.3 |
| 96 | 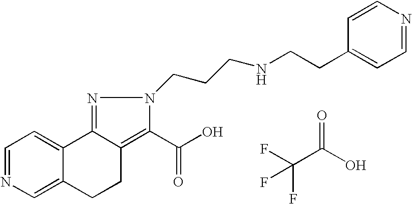 | 2-{3-[(2-pyridin-4-yleth- yl)amino]propyl}-4,5-di- hydro-2H-pyrazolo[3,4-f]iso- quinoline-3-carboxylic acid trifluoroacetate | 1.84 | 35.6 |
| 97 | 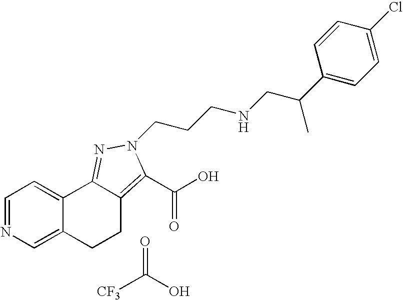 | 2-(3-{[2-(4-chloro- phenyl)propyl]amino}prop- yl)-4,5-dihydro-2H-py- razolo[3,4-f]iso- quinoline-3-carboxylic acid trifluoroacetate | 1.86 | 25.4 |
| 98 | 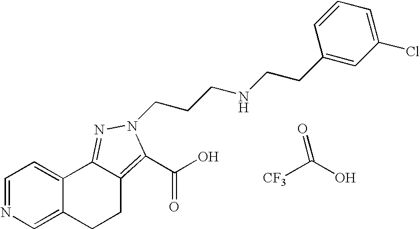 | 2-(3-{[2-(3-chloro- phenyl)ethyl]amino}propyl)-4,5-di- hydro-2H-pyrazolo[3,4-f]iso- quinoline-3-carboxylic acid trifluoroacetate | 2.05 | 48.6 |
| 99 | 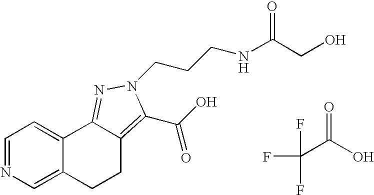 | 2-[3-(glycoloylamino)propyl]-4,5 dihydro-2H-pyrazolo[3,4-f]iso- quinoline-3-carboxylic acid trifluoroacetate | 2.15 | 98.6 |
| 100 | 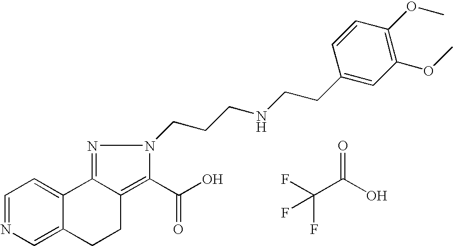 | 2-(3-{[2-(3,4-dimethoxy- phenyl)ethyl]amino}prop- yl)-4,5-dihydro-2H-pyra- zolo[3,4-f]isoquinoline-3-carboxylic acid trifluoroacetate | 2.33 | 48.1 |
| 101 | 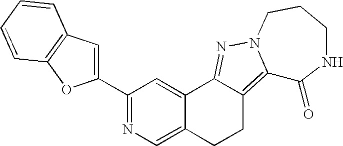 | 2-(1-benzo- furan-2-yl)-5,6,8,9,10,11-hexa- hydro-7H-[1,4]diaze- pino[1′,2′:1,5]pyrazolo[3,4-f]iso- quinolin-7-one | 2.53 | 200 |
| 102 | 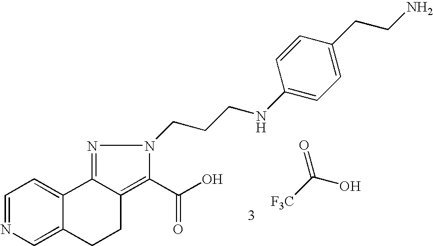 | 2-(3-{[4-(2-amino- ethyl)phenyl]amino}propyl)-4,5-di- hydro-2H-pyrazolo[3,4-f]iso- quinoline-3-carboxylic acid trifluoroacetate | 2.58 | 34.2 |
| 103 | 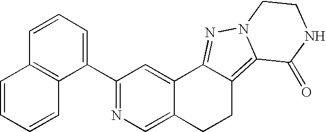 | 2-(1-naphthyl)-5,6,9,10-tetra- hydropyrazino[1′,2′:1,5]pyra- zolo[3,4-f]isoquinolin-7(8H)-one | 2.59 | 1.25 |
| 104 | 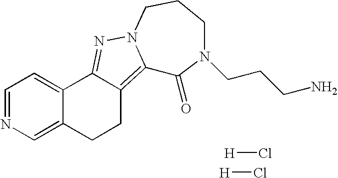 | 8-(3-aminopropyl)-5,6,8,9,10,11 hexahydro-7H-[1,4]diaze- pino[1′,2′:1,5]pyrazolo[4,5-f]iso- quinolin-7-one dihydrochloride | 2.61 | 122 |
| 105 | 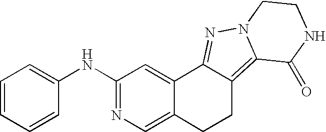 | 2-anilino-5,6,9,10-tetra- hydropyrazino[1′,2′:1,5]pyra- zolo[3,4-f]isoquinolin-7(8H)-one | 2.65 | 2.25 |
| 106 | 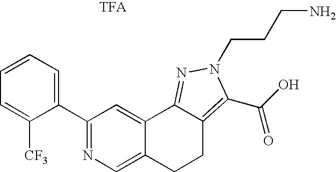 | 2-(3-aminopropyl)-8-[2-(tri- fluoromethyl)phenyl]-4,5-di- hydro-2H-pyrazolo[3,4-f]iso- quinoline-3-carboxylic acid trifluoroacetate | 2.69 | 5.26 |
| 107 | 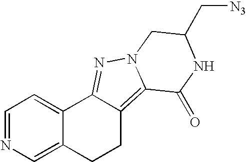 | 9-(azidomethyl)-5,6,9,10-tetra- hydropyrazino[1′,2′:1,5]pyra- zolo[3,4-f]isoquinolin-7(8H)-one | 2.72 | 67.7 |
| 108 | 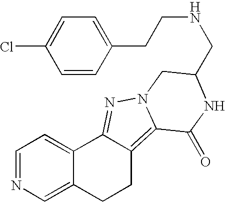 | 9-({[2-(4-chloro- phenyl)ethyl]amino}meth- yl)-5,6,9,10-tetra- hydropyrazino[1′,2′:1,5]pyra- zolo[3,4-f]isoquinolin-7(8H)-one | 2.8 | 120 |
| 109 | 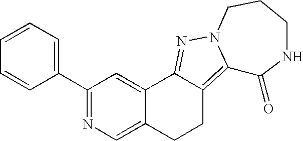 | 2-phenyl-5,6,8,9,10,11-hexa- hydro-7H-[1,4]diaze- pino[1′,2′:1,5]pyrazolo[3,4-f]iso- quinolin-7-one | 2.84 | 200 |
| 110 | 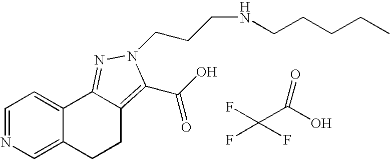 | 2-[3-(pentylamino)propyl]-4,5-di- hydro-2H-pyrazolo[3,4-f]iso- quinoline-3-carboxylic acid trifluoroacetate | 2.86 | 73.3 |
| 111 | 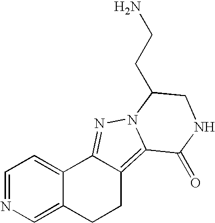 | 10-(2-aminoethyl)-5,6,9,10-tetra- hydropyrazino[1′,2′:1,5]pyra- zolo[3,4-f]isoquinolin-7(8H)-one | 2.94 | 107 |
| 112 | 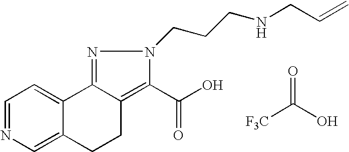 | 2-[3-(allylamino)propyl]-4,5-di- hydro-2H-pyrazolo[3,4-f]iso- quinoline-3-carboxylic acid trifluoroacetate | 3 | 86.3 |
| 113 | 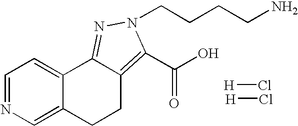 | 2-(4-aminobutyl)-4,5-dihydro-2H pyrazolo[3,4-f]iso- quinoline-3-carboxylic acid dihydrochloride | 3.01 | 174 |
| 114 | 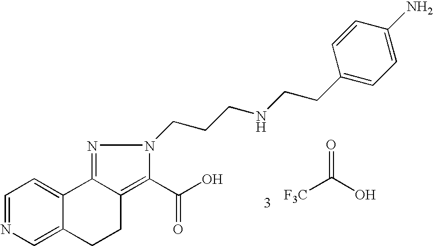 | 2-(3-{[2-(4-amino- phenyl)ethyl]amino}propyl)-4,5-di- hydro-2H-pyrazolo[3,4-f]iso- quinoline-3-carboxylic acid trifluoroacetate | 3.03 | 40.3 |
| 115 | 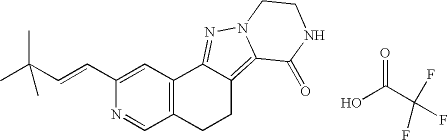 | 2-[(1E)-3,3-dimethyl- but-1-enyl]-5,6,9,10-tetra- hydropyrazino[1′,2′:1,5]pyra- zolo[3,4-f]isoquinolin-7(8H)-one trifluoroacetate | 3.21 | 18.1 |
| 116 | 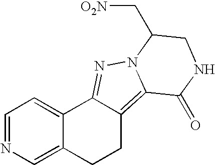 | 10-(nitromethyl)-5,6,9,10-tetra- hydropyrazino[1′,2′:1,5]pyra- zolo[3,4-f]isoquinolin-7(8H)-one | 3.25 | 110 |
| 117 | 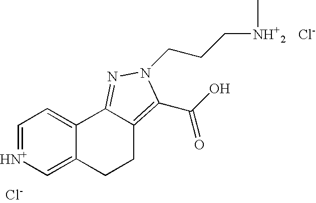 | 3-carboxy-2-[3-(methyl- ammonio)propyl]-4,5-di- hydro-2H-pyrazolo[3,4-f]iso- quinolin-7-ium dichloride | 3.3 | 99 |
| 118 | 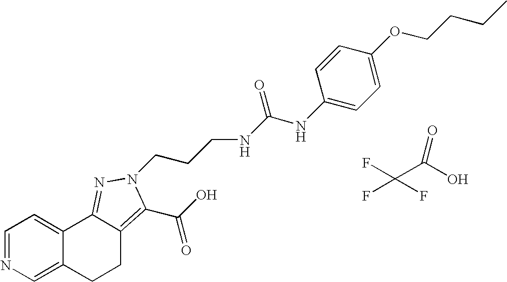 | 2-[3-({[(4-butoxy- phenyl)amino]carbonyl}a- mino)propyl]-4,5-dihydro-2H-py- razolo[3,4-f]iso- quinoline-3-carboxylic acid | 3.39 | 174 |
| 119 | 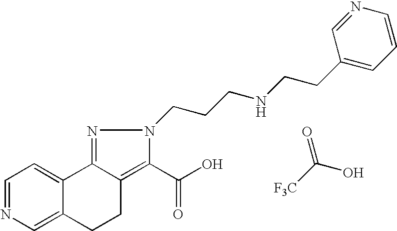 | 2-{3-[(2-pyridin-3-yleth- yl)amino]propyl}-4,5-di- hydro-2H-pyrazolo[3,4-f]iso- quinoline-3-carboxylic acid trifluoroacetate | 3.75 | 64.5 |
| 120 | 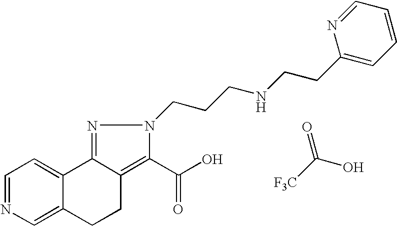 | 2-{3-[(2-pyridin-2-yleth- yl)amino]propyl}-4,5-di- hydro-2H-pyrazolo[3,4-f]iso- quinoline-3-carboxylic acid trifluoroacetate | 3.75 | 44.3 |
| 121 | 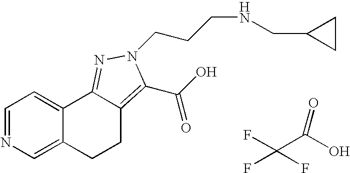 | 2-{3-[(cyclo- propylmethyl)amino]prop- yl}-4,5-di- hydro-2H-pyrazolo[3,4-f]iso- quinoline-3-carboxylic acid trifluoroacetate | 3.94 | 141 |
| 122 | 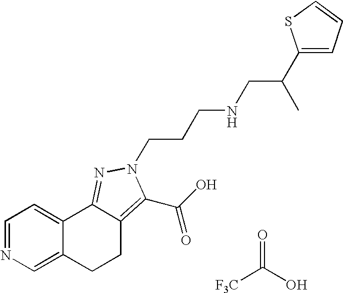 | 2-{3-[(2-thien-2-ylprop- yl)amino]propyl}-4,5-di- hydro-2H-pyrazolo[3,4-f]iso- quinoline-3-carboxylic acid trifluoroacetate | 4.03 | 115 |
| 123 | 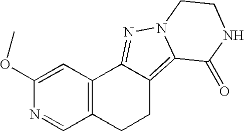 | 2-methoxy-5,6,9,10-tetra- hydropyrazino[1′,2′:1,5]pyra- zolo[3,4-f]isoquinolin-7(8H)-one | 4.05 | 200 |
| 124 | 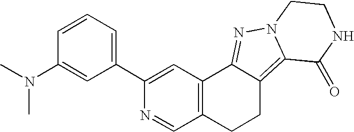 | 2-[3-(dimethyl- amino)phenyl]-5,6,9,10-tetra- hydropyrazino[1′,2′:1,5]pyra- zolo[3,4-f]isoquinolin-7(8H)-one | 4.08 | 51.8 |
| 125 | 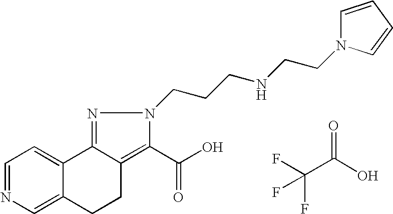 | 2-(3-{[2-(1H-pyrrol-1-yl)eth- yl]amino}propyl)-4,5-di- hydro-2H-pyrazolo[3,4-f]iso- quinoline-3-carboxylic acid trifluoroacetate | 4.26 | 100 |
| 126 | 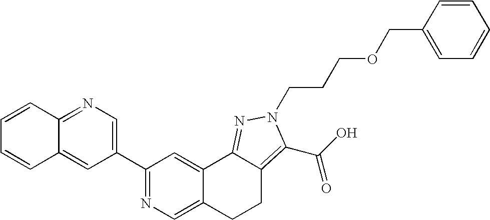 | 2-[3-(benzyloxy)propyl]-8-quino- lin-3-yl-4,5-dihydro-2H-py- razolo[3,4-f]iso- quinoline-3-carboxylic acid | 4.31 | 102 |
| 127 | 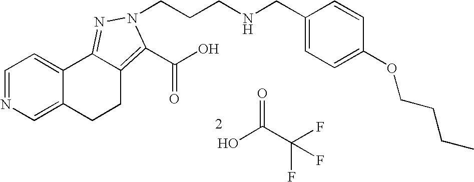 | 2-{3-[(4-butoxy- benzyl)amino]propyl}-4,5-di- hydro-2H-pyrazolo[3,4-f]iso- quinoline-3-carboxylic acid trifluoroacetate | 4.35 | 148 |
| 128 | 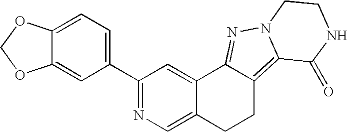 | 2-(1,3-benzo- dioxol-5-yl)-5,6,9,10-tetra- hydropyrazino[1′,2′:1,5]pyra- zolo[3,4-f]isoquinolin-7(8H)-one | 4.44 | 200 |
| 129 | 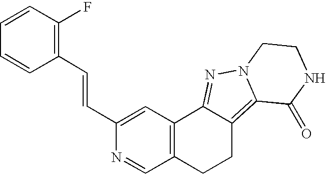 | 2-[(E)-2-(2-fluoro- phenyl)ethenyl]5,6,9,10-tetra- hydropyrazino[1′,2′:1,5]pyra- zolo[3,4-f]isoquinolin-7(8H)-one | 4.46 | 200 |
| 130 | 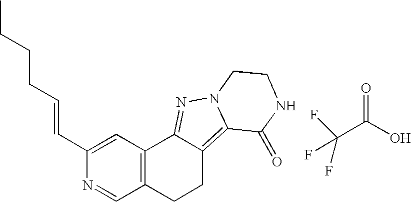 | 2-[(1E)-hex-1-enyl]-5,6,9,10-tetra- hydropyrazino[1′,2′:1,5]pyra- zolo[3,4-f]isoquinolin-7(8H)-one trifluoroacetate | 4.66 | 182 |
| 131 | 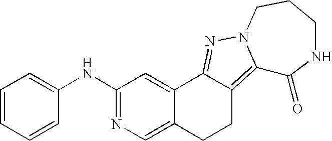 | 2-anilino-5,6,8,9,10,11-hexa- hydro-7H-[1,4]diaze- pino[1′,2′:1,5]pyrazolo[3,4-f]iso- quinolin-7-one | 4.68 | 25.2 |
| 132 | 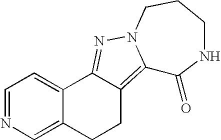 | 5,6,8,9,10,11-hexa- hydro-7H-[1,4]diaze- pino[1′,2′:1,5]pyrazolo[3,4-f]iso- quinolin-7-one | 4.75 | 191 |
| 133 | 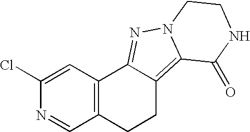 | 2-chloro-5,6,9,10-tetra- hydropyrazino[1′,2′:1,5]pyra- zolo[3,4-f]isoquinolin-7(8H)-one | 4.76 | 200 |
| 134 | 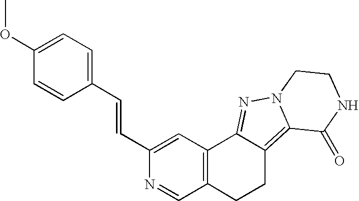 | 2-[(E)-2-(4-methoxy- phenyl)ethenyl]-5,6,9,10-tetra- hydropyrazino[1′,2′:1,5]pyra- zolo[3,4-f]isoquinolin-7(8H)-one | 4.76 | 200 |
| 135 | 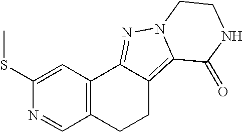 | 2-(methylthio)-5,6,9,10-tetra- hydropyrazino[1′,2′:1,5]pyra- zolo[3,4-f]isoquinolin-7(8H)-one | 4.98 | 200 |
| 136 | 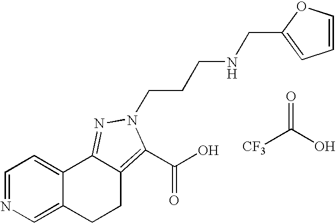 | 2-{3-[(2-furyl- methyl)amino]propyl}-4,5-di- hydro-2H-pyrazolo[3,4-f]iso- quinoline-3-carboxylic acid trifluoroacetate | 5.05 | 57.1 |
| 137 | 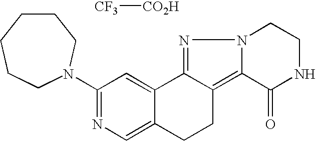 | 2-azepan-1-yl-5,6,9,10-tetra- hydropyrazino[1′,2′:1,5]pyra- zolo[3,4-f]isoquinolin-7(8H)-one trifluoroacetate | 5.12 | 200 |
| 138 | 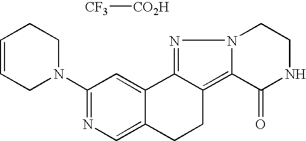 | 2-(3,6-dihydro- pyridin-1(2H)-yl)-5,6,9,10-tetra- hydropyrazino[1′,2′1,5]pyra- zolo[3,4-f]isoquinolin-7(8H)-one trifluoroacetate | 5.28 | 8.73 |
| 139 | 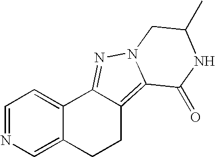 | 9-methyl-5,6,9,10-tetra- hydropyrazino[1′,2′:1,5]pyra- zolo[3,4-f]isoquinolin-7(8H)-one | 5.93 | 200 |
| 140 | 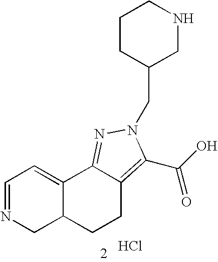 | 2-(piperidin-3-ylmethyl)-4,5-di- hydro-2H-pyrazolo[3,4-f]iso- quinoline-3-carboxylic acid hydrochloride | 5.97 | 200 |
| 141 | 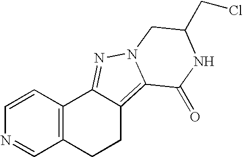 | 9-(chloromethyl)-5,6,9,10-tetra- hydropyrazino[1′,2′:1,5]pyra- zolo[3,4-f]isoquinolin-7(8H)-one | 6.28 | 73.5 |
| 142 | 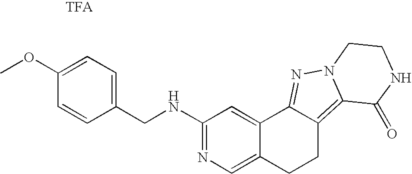 | 2-[(4-methoxy- benzyl)amino]-5,6,9,10-tetra- hydropyrazino[1′,2′:1,5]pyra- zolo]3,4-f]isoquinolin-7(8H)-one trifluoroacetate | 6.37 | 200 |
| 143 | 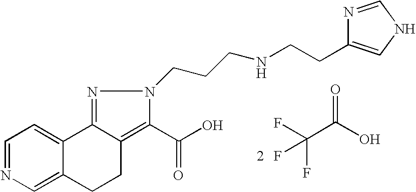 | 2-(3-{[2-(1H-imidazol-4-yl)eth- yl]amino}propyl)-4,5-di- hydro-2H-pyrazolo[3,4-f]iso- quinoline-3-carboxylic acid trifluoroacetate | 6.62 | 136 |
| 144 | 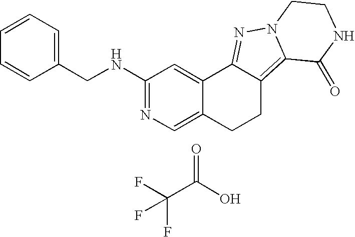 | 2-(benzylamino)-5,6,9,10-tetra- hydropyrazino[1′,2′:1,5]pyra- zolo[3,4-f]isoquinolin-7(8H)-one trifluoroacetate | 6.72 | 200 |
| 145 | 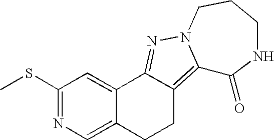 | 2-(methylthio)-5,6,8,9,10,11-hexa- hydro-7H-[1,4]diaze- pino[1′,2′:1,5]pyrazolo[3,4-f]iso- quinolin-7-one | 6.85 | 150 |
| 146 | 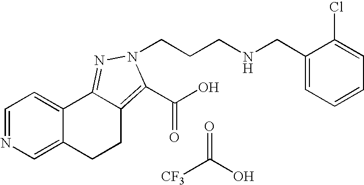 | 2-{3-[(2-chloro- benzyl)amino]propyl}-4,5-di- hydro-2H-pyrazolo[3,4-f]iso- quinoline-3-carboxylic acid trifluoroacetate | 7.02 | 190 |
| 147 | 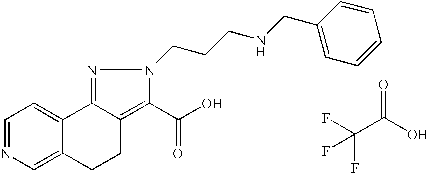 | 2-[3-(benzylamino)propyl]-4,5-di- hydro-2H-pyrazolo[3,4-f]iso- quinoline-3-carboxylic acid trifluoroacetate | 7.13 | |
| 148 | 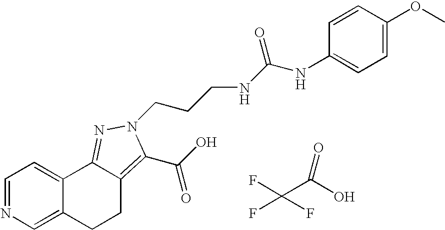 | 2-[3-({[(4-methoxy- phenyl)amino]carbonyl}a- mino)propyl]-4,5-dihydro-2H-py- razolo[3,4-f]isoquinoline-3-car- boxylic acid | 7.32 | 174 |
| 149 | 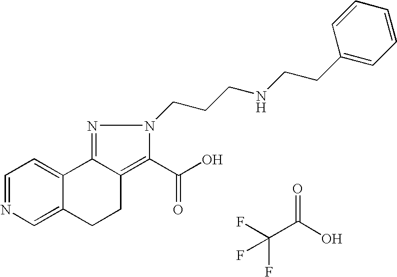 | 2-{3-[(2-phenyl- ethyl)amino]propyl}-4,5-di- hydro-2H-pyrazolo[3,4-f]iso- quinoline-3-carboxylic acid trifluoroacetate | 7.44 | 42.1 |
| 150 | 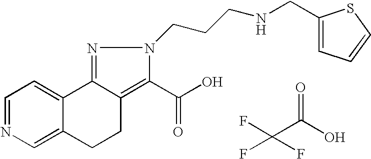 | 2-{3-[(thien-2-ylmeth- yl)amino]propyl}-4,5-di- hydro-2H-pyrazolo[3,4-f]iso- quinoline-3-carboxylic acid trifluoroacetate | 7.79 | 180 |
| 151 | 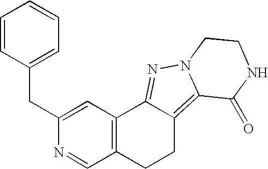 | 2-benzyl-5,6,9,10-tetra- hydropyrazino[1′,2′:1,5]pyra- zolo[3,4-f]isoquinolin-7(8H)-one | 7.86 | 3.38 |
| 152 | 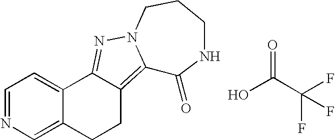 | 5,6,8,9,10,11-hexa- hydro-7H-[1,4]diaze- pino[1′,2′:1,5]pyrazolo[3,4-f]iso- quinolin-7-one | 7.86 | 200 |
| 153 | 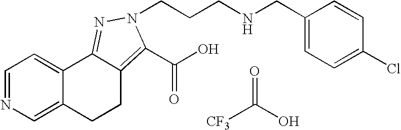 | 2-{3-[(4-chloro- benzyl)amino]propyl}-4,5-di- hydro-2H-pyrazolo[3,4-f]iso- quinoline-3-carboxylic acid trifluoroacetate | 7.96 | 200 |
| 154 | 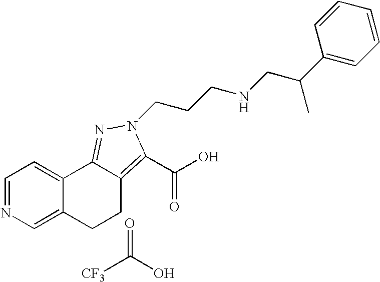 | 2-{3-[(2-phenyl- propyl)amino]propyl}-4,5-di- hydro-2H-pyrazolo[3,4-f]iso- quinoline-3-carboxylic acid trifluoroacetate | 8.28 | 176 |
| 155 | 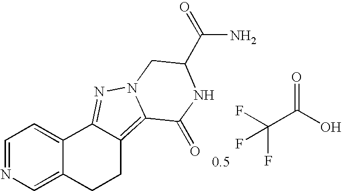 | 7-oxo-5,6,7,8,9,10-hexa- hydropyrazino[1′,2′:1,5]pyra- zolo[3,4-f]iso- quinoline-9-carboxamide trifluoroacetate | 8.76 | 200 |
| 156 | 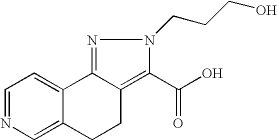 | 2-(3-hydroxypropyl)-4,5-dihydro 2H-pyrazolo[3,4-f]isoquinoline-3 carboxylic acid | 9.43 | 200 |
| 157 | 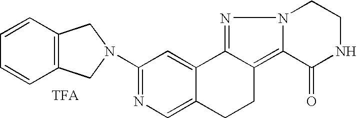 | 2-(1,3-dihydro-2H-iso- indol-2-yl)-5,6,9,10-tetra- hydropyrazino[1′,2′:1,5]pyra- zolo[3,4-f]isoquinolin-7(8H)-one trifluoroacetate | 9.58 | 45.4 |
| 158 | 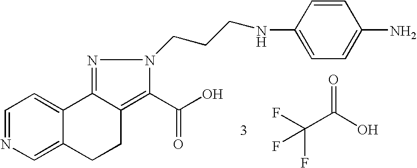 | 2-{3-[(4-amino- phenyl)amino]propyl}-4,5-di- hydro-2H-pyrazolo[3,4-f]iso- quinoline-3-carboxylic acid trifluoroacetate | 9.73 | 133 |
| 159 | 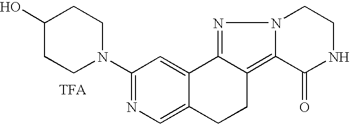 | 2-(4-hydroxy- piperidin-1-yl)-5,6,9,10-tetra- hydropyrazino[1′,2′:1,5]pyra- zolo[3,4-f]isoquinolin-7(8H)-one trifluoroacetate | 9.84 | 22.8 |
| 160 | 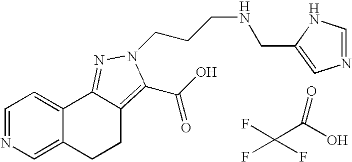 | 2-{3-[(1H-imidazol-5-ylmeth- yl)amino]propyl}-4,5-di- hydro-2H-pyrazolo[3,4-f]iso- quinoline-3-carboxylic acid trifluoroacetate | 10.1 | 200 |
| 161 | 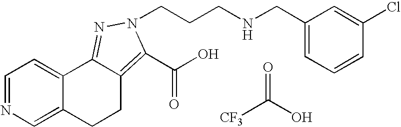 | 2-{3-[(3-chloro- benzyl)amino]propyl}-4,5-di- hydro-2H-pyrazolo[3,4-f]iso- quinoline-3-carboxylic acid trifluoroacetate | 10.3 | 200 |
| 162 | 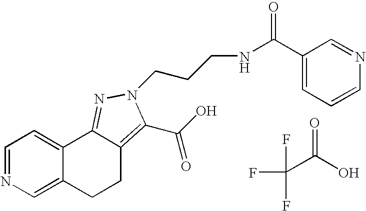 | 2-{3-[(pyridin-3-ylcarbo- nyl)amino]propyl}-4,5-di- hydro-2H-pyrazolo[3,4-f]iso- quinoline-3-carboxylic acid trifluoroacetate | 10.4 | 200 |
| 163 | 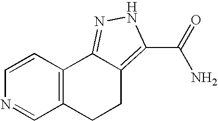 | 4,5-dihydro-2H-pyrazolo[3,4-f]iso- quinoline-3-carboxamide | 10.4 | 200 |
| 164 | 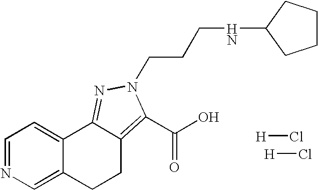 | 2-[3-(cyclo- pentylamino)propyl]-4,5-di- hydro-2H-pyrazolo[3,4-f]iso- quinoline-3-carboxylic acid dihydrochloride | 10.4 | 200 |
| 165 | 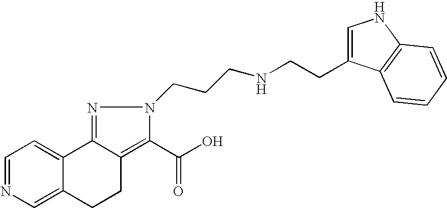 | 2-(3-{[2-(1H-indol-3-yl)eth- yl]amino}propyl)-4,5-di- hydro-2H-pyrazolo[3,4-f]iso- quinoline-3-carboxylic acid | 10.7 | 200 |
| 166 | 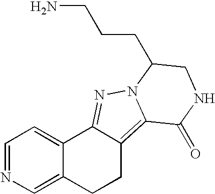 | 10-(3-aminopropyl)-5,6,9,10-tetra- hydropyrazino[1′,2′:1,5]pyra- zolo[3,4-f]isoquinolin-7(8H)-one | 11 | 200 |
| 167 | 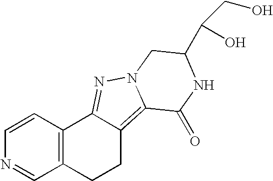 | 9-(1,2-dihydroxyethyl)-5,6,9,10-tetra- hydropyrazino[1′,2′:1,5]pyra- zolo[3,4-f]isoquinolin-7(8H)-one | 11.8 | 200 |
| 168 | 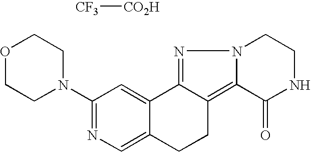 | 2-morpholin-4-yl-5,6,9,10-tetra- hydropyrazino[1′,2′:1,5]pyra- zolo[3,4-f]isoquinolin-7(8H)-one trifluoroacetate | 11.9 | |
| 169 | 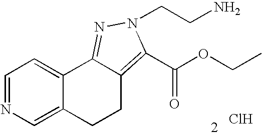 | ethyl 2-(2-aminoethyl)-4,5-di- hydro-2H-pyrazolo[3,4-f]iso- quinoline-3-carboxylate dihydrochloride | 12.3 | 200 |
| 170 | 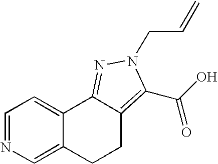 | 2-allyl-4,5-dihydro-2H-py- razolo[3,4-f]iso- quinoline-3-carboxylic acid | 12.6 | 200 |
| 171 | 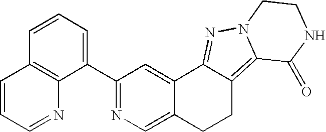 | 2-quinolin-8-yl-5,6,9,10-tetra- hydropyrazino[1′,2′:1,5]pyra- zolo[3,4-f]isoquinolin-7(8H)-one | 14 | 74.8 |
| 172 | 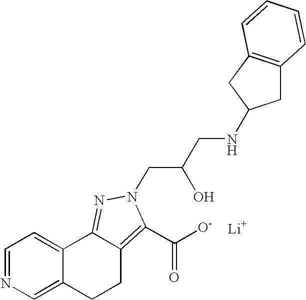 | lithium 2-[3-(2,3-dihydro-1H-in- den-2-ylamino)-2-hydroxy- propyl]-4,5-dihydro-2H-py- razolo[3,4-f]isoquinoline-3-car- boxylate | 14 | |
| 173 | 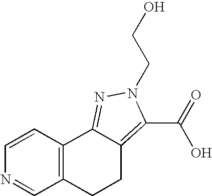 | 2-(2-hydroxy- ethyl)-4,5-dihydro-2H-py- razolo[3,4-f]isoquinoline-3 carboxylic acid | 14.3 | 200 |
| 174 | 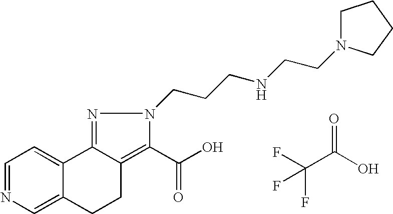 | 2-{3-[(2-pyrrolidin-1-yleth- yl)amino]propyl}-4,5-di- hydro-2H-pyrazolo[3,4-f]iso- quinoline-3-carboxylic acid trifluoroacetate | 14.6 | 200 |
| 175 | 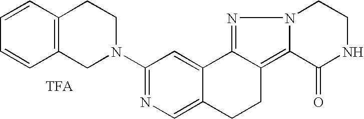 | 2-(3,4-dihydro- isoquinolin-2(1H)-yl)-5,6,9,10-tetra- hydropyrazino[1′,2′:1,5]pyra- zolo[3,4-f]isoquinolin-8(7H)-one trifluoroacetate | 15 | |
| 176 | 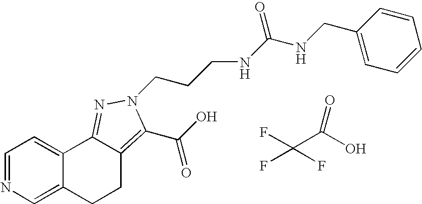 | 2-(3-{[(benzyl- amino)carbonyl]amino}pro- pyl)-4,5-dihydro-2H-py- razolo[3,4-f]iso- quinoline-3-carboxylic acid | 15.4 | 200 |
| 177 | 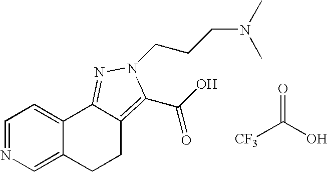 | 2-[3-(dimethylamino)propyl]-4,5-di- hydro-2H-pyrazolo[3,4-f]iso- quinoline-3-carboxylic acid trifluoroacetate | 15.5 | 200 |
| 178 | 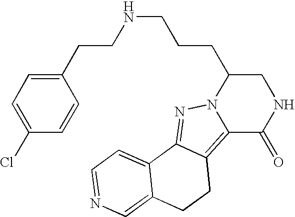 | 10-(3-{[2-(4-chloro- phenyl)ethyl]a- mino}propyl)-5,6,9,10-tetra- hydropyrazino[1′,2′:1,5]pyra- zolo[3,4-f]isoquinolin-7(8H)-one | 16.1 | 40.4 |
| 179 | 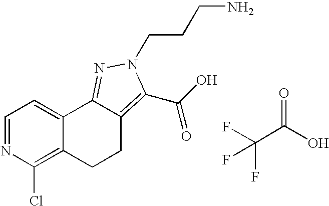 | 2-(3-aminopropyl)-6-chloro-4,5-di- hydro-2H-pyrazolo[3,4-f]iso- quinoline-3-carboxylic acid trifluoroacetate | 16.5 | 200 |
| 180 | 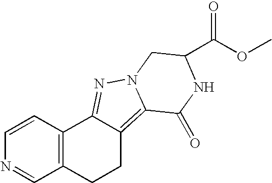 | methyl 7-oxo-5,6,7,8,9,10-hexa- hydropyrazino[1′,2′:1,5]pyra- zolo[3,4-f]isoquinoline-9-car- boxylate | 16.5 | |
| 181 | 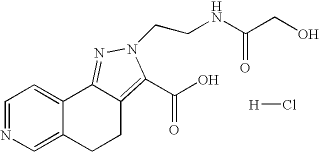 | 2-[2-(glycoloylamino)ethyl]-4,5-di- hydro-2H-pyrazolo[3,4-f]iso- quinoline-3-carboxylic acid hydrochloride | 17.4 | 200 |
| 182 | 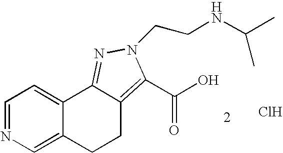 | 2-[2-(isopropylamino)ethyl]-4,5-di- hydro-2H-pyrazolo[3,4-f]iso- quinoline-3-carboxylic acid hydrochloride | 17.7 | 200 |
| 183 | 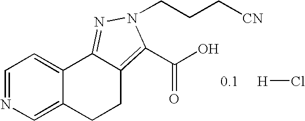 | 2-(3-cyano- propyl)-4,5-dihydro-2H-py- razolo[3,4-f]isoquinoline-3 carboxylic acid | 17.9 | 200 |
| 184 | 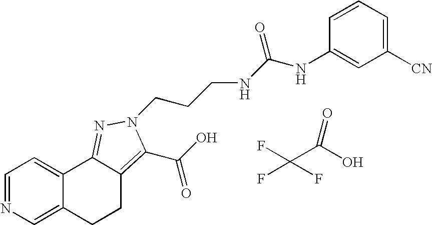 | 2-[3-({[(3-cyano- phenyl)amino]carbonyl}a- mino)propyl]-4,5-dihydro-2H-py- razolo[3,4-f]iso- quinoline-3-carboxylic acid | 17.9 | 200 |
| 185 | 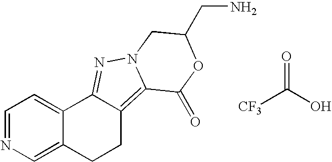 | 9-(aminomethyl)-5,6,9,10-tetra- hydro-7H-[1,4]oxa- zino[4′,3′:1,5]pyrazolo[3,4-f]iso- quinolin-7-one trifluoroacetate | 19.4 | |
| 186 | 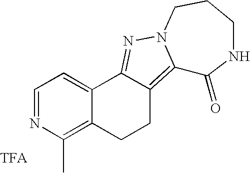 | 4-methyl-5,6,8,9,10,11-hexa- hydro-7H-[1,4]diaze- pino[1′,2′:1,5]pyrazolo[3,4-f]iso- quinolin-7-one trifluoroacetate | 20 | |
| 187 | 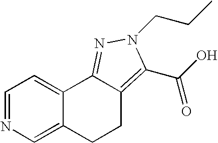 | 2-propyl-4,5-dihydro-2H-pyra- zolo[3,4-f]isoquinoline-3-carboxylic acid | 20.1 | |
| 188 | 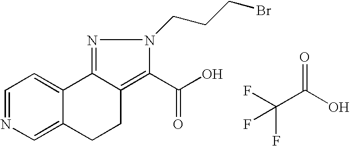 | 2-(3-bromo- propyl)-4,5-dihydro-2H-py- razolo[3,4-f]isoquinoline-3 carboxylic acid trifluoroacetate | 21.5 | 200 |
| 189 | 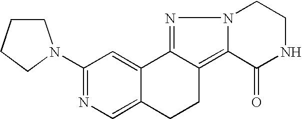 | 2-pyrrolidin-1-yl-5,6,9,10-tetra- hydropyrazino[1′,2′:1,5]pyra- zolo[3,4-f]isoquinolin-7(8H)-one | 21.6 | |
| 190 | 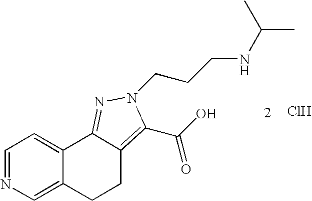 | 2-[3-(isopropylamino)propyl]-4,5 dihydro-2H-pyrazolo[3,4-f]iso- quinoline-3-carboxylic acid hydrochloride | 21.9 | |
| 191 | 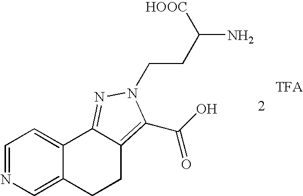 | 2-(3-amino-3-carboxypropyl)-4,5-di- hydro-2H-pyrazolo[3,4-f]iso- quinoline-3-carboxylic acid trifluoroacetate | 25 | 200 |
| 192 | 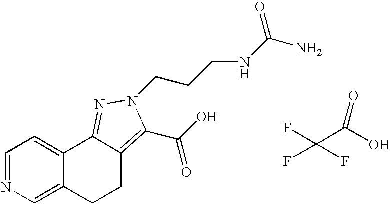 | 2-{3-[(amino- carbonyl)amino]propyl}-4,5-di- hydro-2H-pyrazolo[3,4-f]iso- quinoline-3-carboxylic acid | 25.3 | 200 |
| 193 | 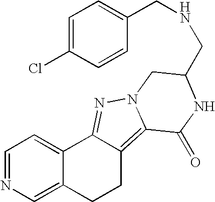 | 9-{[(4-chloro- benzyl)amino]methyl}-5,6,9,10-tetra- hydropyrazino[1′,2′:1,5]pyra- zolo[3,4-f]isoquinolin-7(8H)-one | 27.3 | 200 |
| 194 | 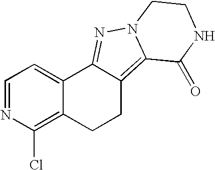 | 4-chloro-5,6,9,10-tetra- hydropyrazino[1′,2′:1,5]pyra- zolo[3,4-f]isoquinolin-7(8H)-one | 27.7 | 200 |
| 195 | 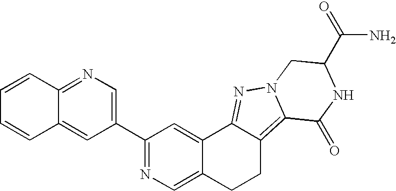 | 7-oxo-2-quino- lin-3-yl-5,6,7,8,9,10-hexa- hydropyrazino[1′,2′:1,5]pyra- zolo[3,4-f]iso- quinoline-9-carboxamide | 27.9 | |
| 196 | 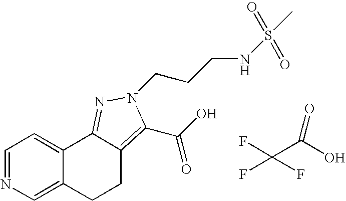 | 2-{3-[(methyl- sulfonyl)amino]propyl}-4,5-di- hydro-2H-pyrazolo[3,4-f]iso- quinoline-3-carboxylic acid trifluoroacetate | 28.2 | 200 |
| 197 | 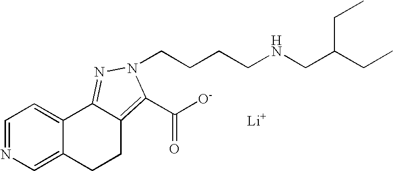 | lithium 2-{4-[(2-ethyl- butyl)amino]butyl}-4,5-di- hydro-2H-pyrazolo[3,4-f]iso- quinoline-3-carboxylate | 29.4 | |
| 198 | 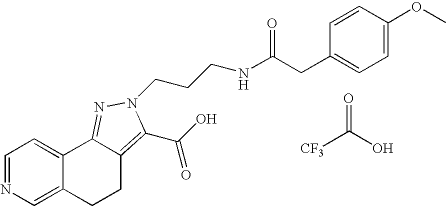 | 2-(3-{[(4-methoxy- phenyl)acetyl]amino}pro- pyl)-4,5-dihydro-2H-pyra- zolo[3,4-f]isoquinoline-3-carboxylic acid trifluoroacetate | 30.1 | |
| 199 | 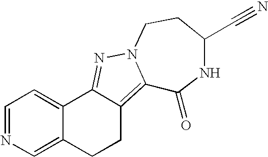 | 7-oxo-6,7,8,9,10,11-hexa- hydro-5H-[1,4]diaze- pino[1′,2′:1,5]pyrazolo[3,4-f]iso- quinoline-9-carbonitrile | 33.3 | |
| 200 | 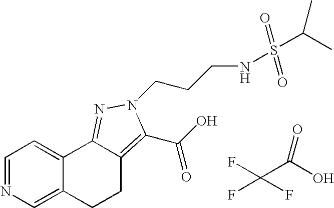 | 2-{3-[(isopropyl- sulfonyl)amino]propyl}-4,5-di- hydro-2H-pyrazolo[3,4-f]iso- quinoline-3-carboxylic acid trifluoroacetate | 33.3 | 200 |
| 201 | 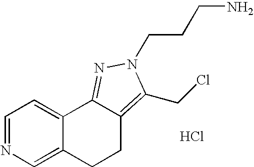 | 3-[3-(chloro- methyl)-4,5-dihydro-2H-pyra- zolo[3,4-f]isoquinolin-2-yl]pro- pan-1-amine hydrochloride | 33.5 | |
| 202 | 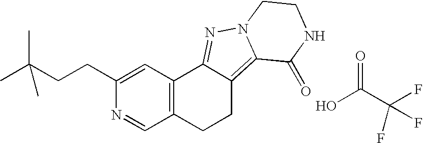 | 2-(3,3-dimethylbutyl)-5,6,9,10-tetra- hydropyrazino[1′,2′:1,5]pyra- zolo[3,4-f]iso- quinolin-7(8H)-one trifluoroacetate | 35.6 | |
| 203 | 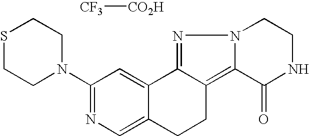 | 2-thiomorpholin-4-yl-5,6,9,10-tetra- hydropyrazino[1′,2′:1,5]pyra- zolo[3,4-f]iso- quinolin-7(8H)-one trifluoroacetate | 35.6 | 3.3 |
| 204 | 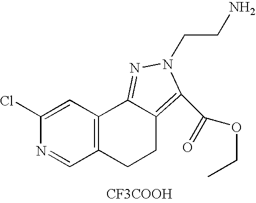 | ethyl 2-(2-amino- ethyl)-8-chloro-4,5-di- hydro-2H-pyrazolo[3,4-f]iso- quinoline-3-carboxylate | 36 | 200 |
| 205 | 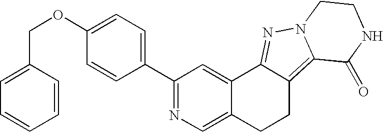 | 2-[4-(benzyl- oxy)phenyl]-5,6,9,10-tetra- hydropyrazino[1′,2′:1,5]pyra- zolo[3,4-f]isoquinolin-7(8H)-one | 36.4 | |
| 206 | 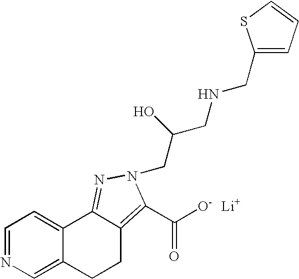 | lithium 2-{2-hy- droxy-3-[(thien-2-ylmeth- yl)amino]propyl}-4,5-di- hydro-2H-pyrazolo[3,4-f]iso- quinoline-3-carboxylate | 36.8 | |
| 207 | 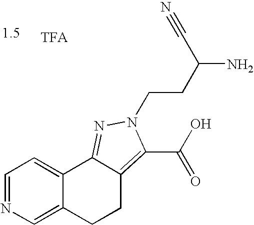 | 2-(3-amino-3-cyanopropyl)-4,5-di- hydro-2H-pyrazolo[3,4-f]iso- quinoline-3-carboxylic acid trifluoroacetate | 36.9 | 186 |
| 208 | 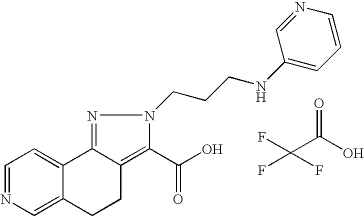 | 2-[3-(pyri- din-3-ylamino)propyl]-4,5-di- hydro-2H-pyrazolo[3,4-f]iso- quinoline-3-carboxylic acid trifluoroacetate | 39.4 | 200 |
| 209 | 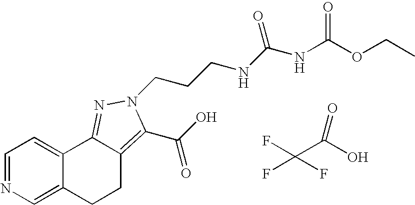 | 2-[3-({[(ethoxy- carbonyl)amino]carbo- nyl}amino)propyl]-4,5-di- hydro-2H-py- razolo[3,4-f]isoquinoline-3 carboxylic acid trifluoroacetate | 42.5 | 200 |
| 210 | 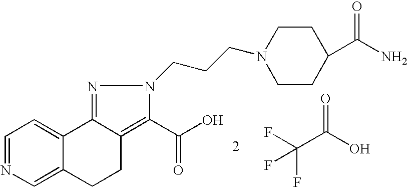 | 2-{3-[4-(aminocarbonyl)piperidin 1-yl]propyl}-4,5-dihydro-2H-pyra- zolo[3,4-f]isoquinoline-3-carboxylic acid trifluoroacetate | 47.9 | |
| 211 | 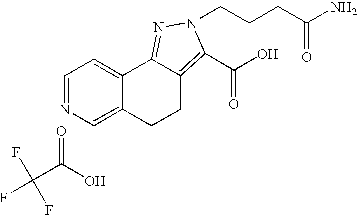 | 2-(4-amino-4-oxobutyl)-4,5-di- hydro-2H-pyrazolo[3,4-f]iso- quinoline-3-carboxylic acid trifluoroacetate | 50.1 | |
| 212 | 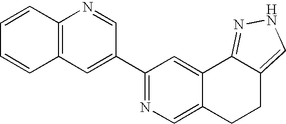 | 8-quinolin-3-yl-4,5-dihydro-2H-py- razolo[3,4-f]isoquinoline | 52 | |
| 213 | 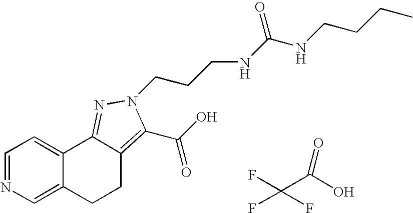 | 2-(3-{[(butyl- amino)carbonyl]amino}pro- pyl)-4,5-dihydro-2H-py- razolo[3,4-f]iso- quinoline-3-carboxylic acid trifluoroacetate | 53.5 | 200 |
| 214 | 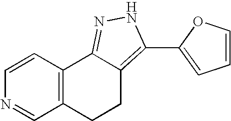 | 3-(2-furyl)-4,5-dihydro-2H-pyra- zolo[3,4-f]isoquinoline | 53.7 | 200 |
| 215 | 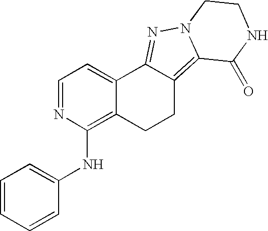 | 4-anilino-5,6,9,10-tetra- hydropyrazino[1′,2′:1,5]pyra- zolo[3,4-f]isoquinolin-7(8H)-one | 53.8 | 200 |
| 216 | 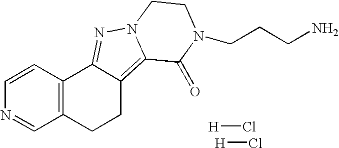 | 8-(3-aminopropyl)-5,6,9,10-tetra- hydropyrazino[1′,2′:1,5]pyra- zolo[3,4-f]isoquinolin-7(8H)-one dihydrochloride | 54.8 | 200 |
| 217 | 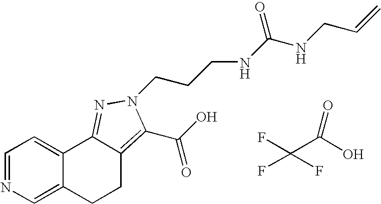 | 2-(3-{[(allyl- amino)carbonyl]amino}pro- pyl)-4,5-dihydro-2H-py- razolo[3,4-f]iso- quinoline-3-carboxylic acid trifluoroacetate | 55.3 | 200 |
| 218 | 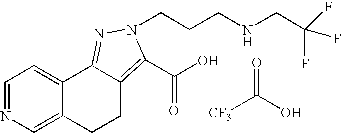 | 2-{3-[(2,2,2-tri- fluoroethyl)amino]propyl}-4,5-di- hydro-2H-pyrazolo[3,4-f]iso- quinoline-3-carboxylic acid trifluoroacetate | 56.3 | 200 |
| 219 | 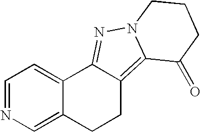 | 5,6,9,10-tetra- hydropyrido[1′,2′:1.5]pyra- zolo[3,4-f]isoquinolin-7(8H)-one | 59.5 | |
| 220 | 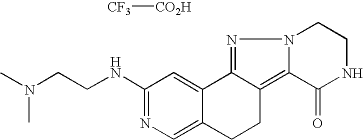 | 2-{[2-(dimethyl- amino)ethyl]amino}-5,6,9,10-tetra- hydropyrazino[1′,2′:1.5]pyra- zolo[3,4-f]isoquinolin-7(8H)-one trifluoroacetate | 60.7 | |
| 221 | 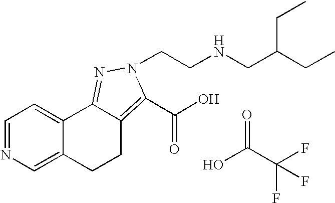 | 2-{2-[(2-ethyl- butyl)amino]ethyl}-4,5-di- hydro-2H-pyrazolo[3,4-f]iso- quinoline-3-carboxylic acid | 62.1 | |
| 222 | 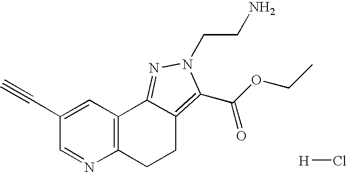 | ethyl 2-(2-aminoethyl)-8-ethynyl 4,5-dihydro-2H-pyrazolo[3,4-f]iso- quinoline-3-carboxylate hydrochloride | 62.5 | |
| 223 | 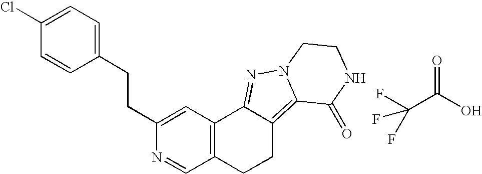 | 2-[2-(4-chloro- phenyl)ethyl]-5,6,9,10-tetra- hydropyrazino[1′,2′:1.5]pyra- zolo[3,4-f]isoquinolin-7(8H)-one trifluoroacetate | 63.2 | |
| 224 | 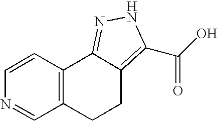 | 4,5-dihydro-2H-pyrazolo[3,4-f]iso- quinoline-3-carboxylic acid | 63.4 | 200 |
| 225 | 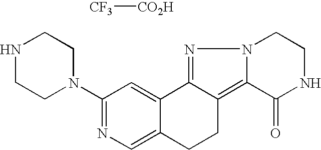 | 2-piperazin-1-yl-5,6,9,10-tetra- hydropyrazino[1′,2′:1.5]pyra- zolo[3,4-f]isoquinolin-7(8H)-one trifluoroacetate | 64.9 | |
| 226 | 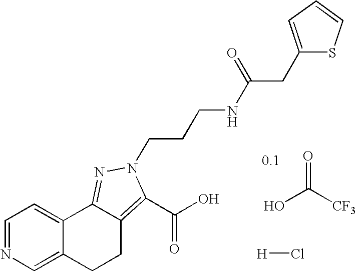 | 2-{3-[(thien-2-ylace- yl)amino]propyl}-4,5-di- hydro-2H-pyrazolo[3,4-f]iso- quinoline-3-carboxylic acid trifluoroacetate hydrochloride | 66.1 | 200 |
| 227 | 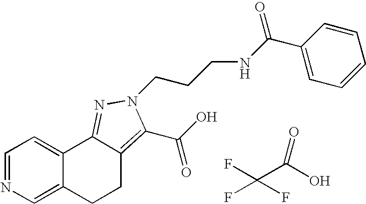 | 2-[3-(benzoylamino)propyl]-4,5-di- hydro-2H-pyrazolo[3,4-f]iso- quinoline-3-carboxylic acid trifluoroacetate | 68 | 200 |
| 228 | 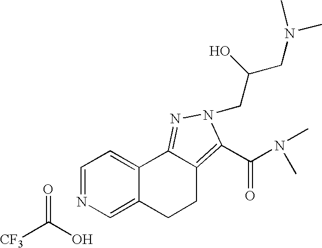 | 2-[3-(dimethylamino)-2-hydroxy- propyl]-N,N-dimethyl-4,5-di- hydro-2H-pyrazolo[3,4-f]iso- quinoline-3-carboxamide trifluoroacetate | 69.3 | |
| 229 | 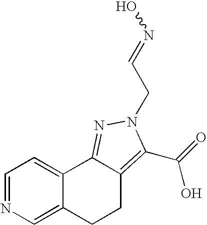 | 2-[(2E)-2-(hydroxy- imino)ethyl]-4,5-di- hydro-2H-pyrazolo[3,4-f]iso- quinoline-3-carboxylic acid | 71 | |
| 230 | 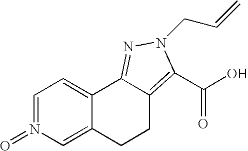 | 2-allyl-4,5-dihydro-2H-py- razolo[3,4-f]iso- quinoline-3-carboxylic acid 7-oxide | 75.4 | 200 |
| 231 | 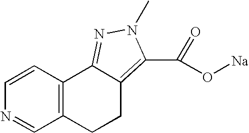 | | 76.6 | 200 |
| 232 | 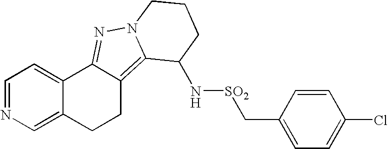 | | 78.7 | |
| 233 | 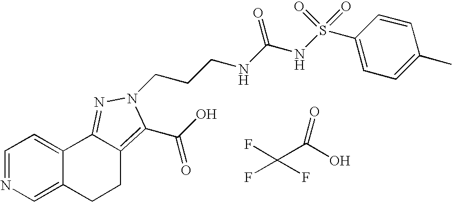 | 2-{3-[({[(4-methyl- phenyl)sulfonyl]amino}car- bonyl)amino]propyl}-4,5-di- hydro-2H-pyrazolo[3,4-f]iso- quinoline-3-carboxylic acid trifluoroacetate | 80.6 | 200 |
| 234 | 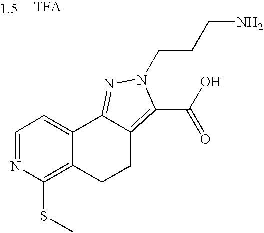 | 2-(3-aminopropyl)-6-(methyl- thio)-4,5-dihydro-2H-py- razolo[3,4-f]iso- quinoline-3-carboxylic acid trifluoroacetate | 83.4 | 200 |
| 235 | 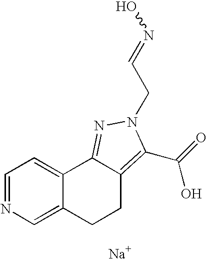 | For multi-component charged structures, a total zero charge is required! | 84.3 | |
| 236 | 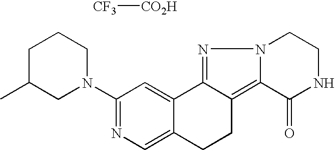 | 2-(3-methyl- piperidin-1-yl)-5,6,9,10-tetra- hydropyrazino[1′,2′:1,5]pyra- zolo[3,4-f]isoquinolin-7(8H)-one trifluoroacetate | 84.4 | |
| 237 | 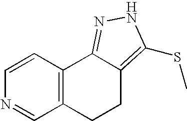 | 3-(methylthio)-4,5-dihydro-2H-pyra- zolo[3,4-f]isoquinoline | 85.2 | 200 |
| 238 | 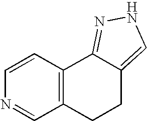 | | 86.1 | 200 |
| 239 | 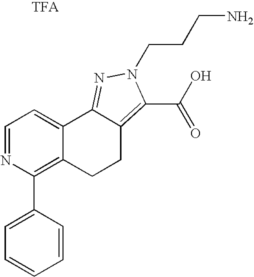 | 2-(3-aminopropyl)-6-phenyl-4,5-di- hydro-2H-pyrazolo[3,4-f]iso- quinoline-3-carboxylic acid trifluoroacetate | 87.6 | 200 |
| 240 | 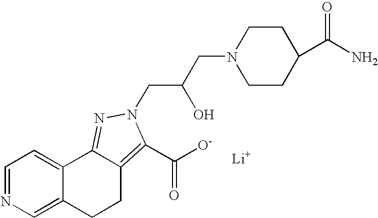 | lithium 2-{3-[4-(amino- carbonyl)piperidin-1-yl]-2 hydroxypropyl}-4,5-di- hydro-2H-pyra- zolo[3,4-f]isoquinoline-3-carboxylate | 87.6 | |
| 241 | 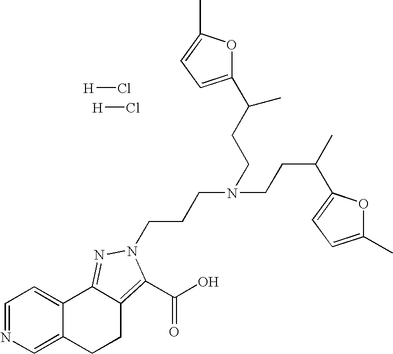 | 2-(3-{bis[3-(5-methyl-2-furyl)bu- tyl]amino}propyl)-4,5-di- hydro-2H-pyrazolo[3,4-f]iso- quinoline-3-carboxylic acid dihydrochloride | 87.9 | 200 |
| 242 | 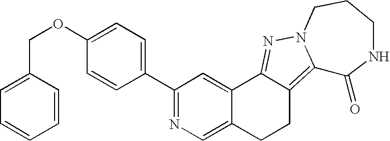 | 2-[4-(benzyl- oxy)phenyl]-5,6,8,9,10,11-hexa- hydro-7H-[1,4]diaze- pino[1′,2′:1,5]pyrazolo[3,4-f]iso- quinolin-7-one | 93.4 | |
| 243 | 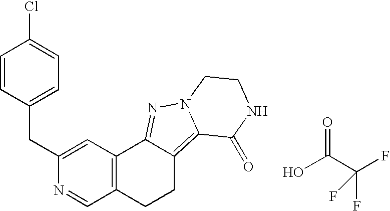 | 2-(4-chlorobenzyl)-5,6,9,10-tetra- hydropyrazino[1′,2′:1,5]pyra- zolo[3,4-f]isoquinolin-7(8H)-one trifluoroacetate | 94.2 | 200 |
| 244 | 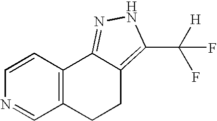 | 3-(difluoro- methyl)-4,5-dihydro-2H-pyra- zolo[3,4-f]isoquinoline | 96.5 | 200 |
| 245 | 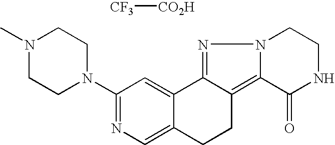 | 2-(4-methyl- piperazin-1-yl)-5,6,9,10-tetra- hydropyrazino[1′,2′:1.5]pyra- zolo[3,4-f]isoquinolin-7(8H)-one trifluoroacetate | 96.8 | 108 |
| 246 | 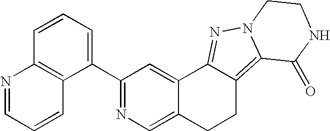 | 2-quinolin-5-yl-5,6,9,10-tetra- hydropyrazino[1′,2′:1.5]pyra- zolo[3,4-f]isoquinolin-7(8H)-one | 98.3 | |
| 247 | 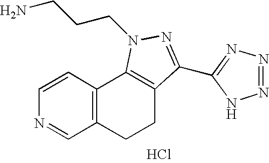 | 3-[3-(1H-tetraazol-5-yl)-4,5-di- hydro-1H-pyrazolo[3,4-f]iso- quinolin-1-yl]propan-1-amine hydrochloride | 100 | 200 |
| 248 | 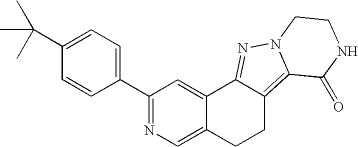 | 2-(4-tert-butylphenyl)-5,6,9,10-tetra- hydropyrazino[1′,2′:1.5]pyra- zolo[3,4-f]isoquinolin-7(8H)-one | 101 | |
| 249 | 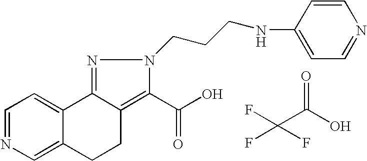 | 2-[3-(py- ridin-4-ylamino)propyl]-4,5-di- hydro-2H-pyrazolo[3,4-f]iso- quinoline-3-carboxylic acid | 101 | |
| 250 | 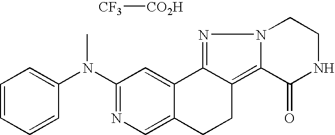 | 2-[meth- yl(phenyl)amino]-5,6,9,10-tetra- hydropyrazino[1′,2′:1.5]pyra- zolo[3,4-f]isoquinolin-7(8H)-one trifluoroacetate | 101 | |
| 251 | 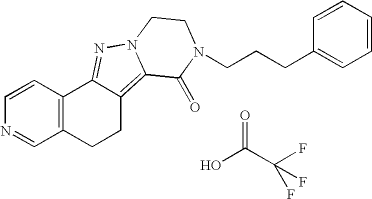 | 8-(3-phenylpropyl)-5,6,9,10-tetra- hydropyrazino[1′,2′:1.5]pyra- zolo[3,4-f]isoquinolin-7(8H)-one trifluoroacetate | 102 | 200 |
| 252 | 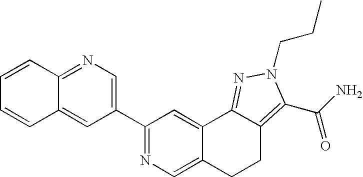 | 2-propyl-8-quinolin-3-yl-4,5-di- hydro-2H-pyrazolo[3,4-f]iso- quinolin-3-carboxamide | 103 | |
| 253 | 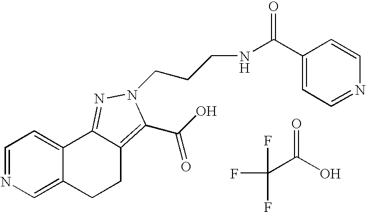 | 2-[3-(isonicotinoyl- amino)propyl]-4,5-di- hydro-2H-pyrazolo[3,4-f]iso- quinoline-3-carboxylic acid trifluoroacetate | 109 | 200 |
| 254 | 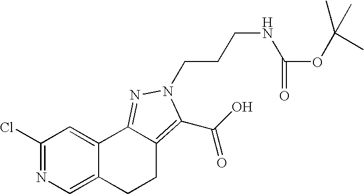 | 2-{3-[(tert- butoxycarbonyl)amino]propyl}-8 chloro-4,5-dihydro-2H-py- razolo[3,4-f]iso- quinoline-3-carboxylic acid | 112 | 200 |
| 255 | 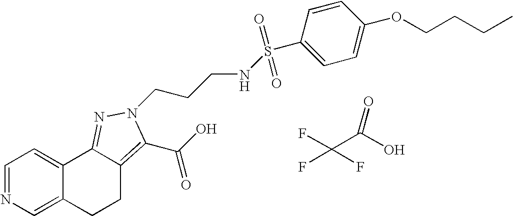 | 2-(3-{[(4-butoxy- phenyl)sulfonyl]amino}pro- pyl)-4,5-dihydro-2H-pyra- zolo[3,4-f]isoquinoline-3-carboxylic acid trifluoroacetate | 113 | 200 |
| 256 | 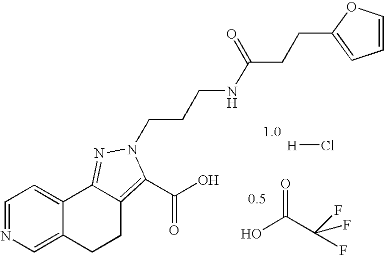 | 2-(3-{[3-(2-furyl)propan- oyl]amino}propyl)-4,5-di- hydro-2H-pyrazolo[3,4-f]iso- quinoline-3-carboxylic acid trifluoroacetate hydrochloride | 116 | 200 |
| 257 | 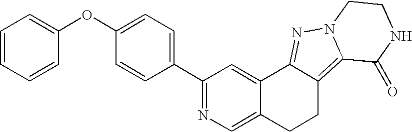 | 2-(4-phenoxyphenyl)-5,6,9,10-tetra- hydropyrazino[1′,2′:1,5]pyra- zolo[3,4-f]isoquinolin-7(8H)-one | 119 | |
| 258 | 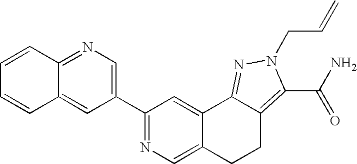 | 2-allyl-8-quinolin-3-yl-4,5-di- hydro-2H-pyrazolo[3,4-f]iso- quinolin-3-carboxamide | 119 | |
| 259 | 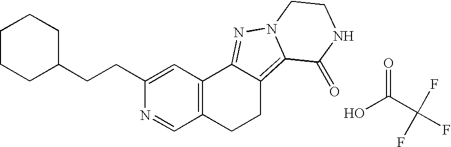 | 2-(2-cyclohexylethyl)-5,6,9,10-tetra- hydropyrazino[1′,2′:1,5]pyra- zolo[3,4-f]isoquinolin-7(8H)-one trifluoroacetate | 121 | |
| 260 | 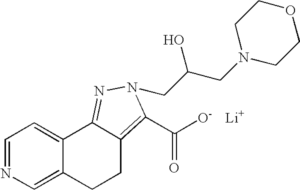 | lithium 2-(2-hydroxy-3-morpho- lin-4-ylpropyl)-4,5-di- hydro-2H-pyrazolo[3,4-f]iso- quinoline-3-carboxylate | 128 | |
| 261 | 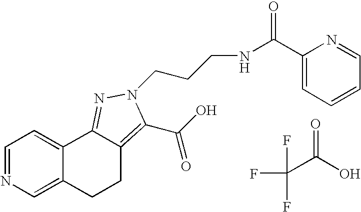 | 2-{3-[(pyridin-2-ylcarbo- nyl)amino]propyl}-4,5-di- hydro-2H-pyrazolo[3,4-f]iso- quinoline-3-carboxylic acid trifluoroacetate | 129 | 200 |
| 262 | 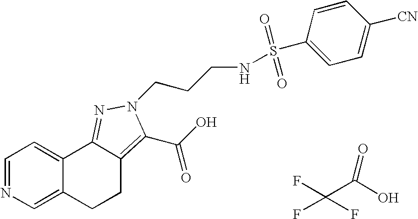 | 2-(3-{[(4-cyano- phenyl)sulfonyl]amino}pro- pyl)-4,5-dihydro-2H-pyra- zolo[3,4-f]isoquinoline-3-carboxylic acid trifluoroacetate | 130 | 200 |
| 263 | 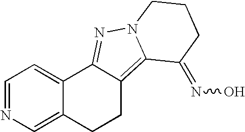 | (7E)-5,6,9,10-tetra- hydropyrido[1′,2′:1,5]pyrazo- lo[3,4-f]isoquinolin-7(8H)-one oxime | 131 | |
| 264 | 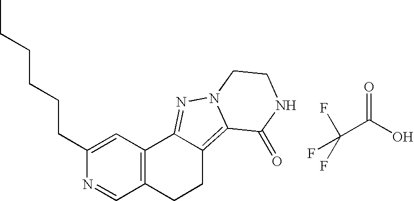 | 2-hexyl-5,6,9,10-tetra- hydropyrazino[1′,2′:1,5]pyra- zolo[3,4-f]isoquinolin-7(8H)-one trifluoroacetate | 133 | |
| 265 | 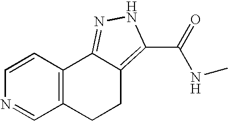 | N-methyl-4,5-dihydro-2H-py- razolo[3,4-f]iso- quinoline-3-carboxamide | 135 | 200 |
| 266 | 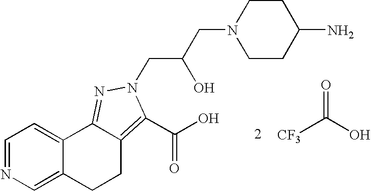 | 2-[3-(4-amino- piperidin-1-yl)-2-hydroxy- propyl]-4,5-dihydro-2H-py- razolo[3,4-f]isoquinolin-3-carboxylic acid bis(trifluoroacetate) | 136 | |
| 267 | 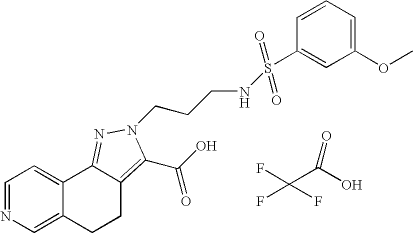 | 2-(3-{[(3-methoxy- phenyl)sulfonyl]amino}pro- pyl)-4,5-dihydro-2H-py- razolo[3,4-f]iso- quinoline-3-carboxylic acid trifluoroacetate | 141 | 200 |
| 268 | 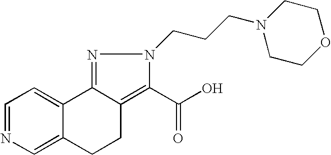 | 2-(3-morpholin-4-ylpropyl)-4,5-di- hydro-2H-pyrazolo[3,4-f]iso- quinoline-3-carboxylic acid | 143 | |
| 269 | 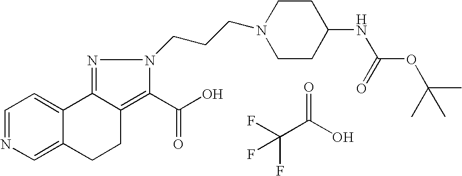 | 2-(3-{4-[(tert- butoxycarbonyl)a- mino]piperidin-1-yl}pro- pyl)-4,5-dihydro-2H-py- razolo[3,4-f]iso- quinoline-3-carboxylic acid trifluoroacetate | 149 | |
| 270 | 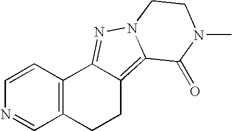 | 8-methyl-5,6,9,10-tetra- hydropyrazino[1′,2′:1,5]pyra- zolo[3,4-f]isoquinolin-7(8H)-one | 156 | 200 |
| 271 | 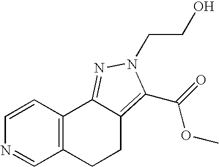 | methyl 2-(2-hydroxyethyl)-4,5-di- hydro-2H-pyrazolo[3,4-f]iso- quinoline-3-carboxylate | 161 | 200 |
| 272 | 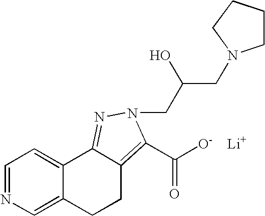 | lithium 2-(2-hydroxy-3-pyrrolidin 1-ylpropyl)-4,5-dihydro-2H-py- razolo[3,4-f]isoquino- line-3-carboxylate | 162 | |
| 273 | 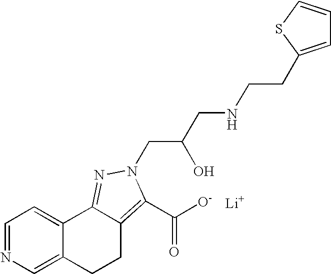 | lithium 2-{2-hy- droxy-3-[(2-thien-2-yleth- yl)amino]propyl}-4,5-di- hydro-2H-pyrazolo[3,4-f]iso- quinoline-3-carboxylate | 163 | |
| 274 | 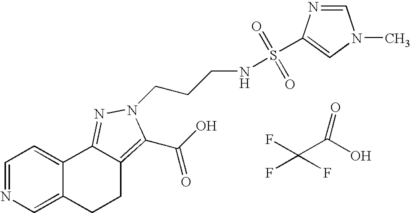 | 2-(3-{[(1-meth- yl-1H-imidazol-4-yl)sulfo- nyl]amino}propyl)-4,5-di- hydro-2H-pyrazolo[3,4-f]iso- quinoline-3-carboxylic acid trifluoroacetate | 164 | 200 |
| 275 | 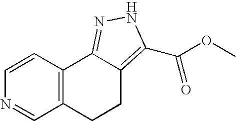 | | 171 | 200 |
| 276 | 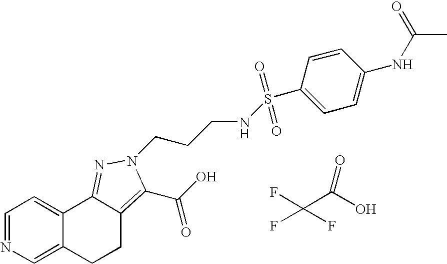 | 2-[3-({[4-(acetyl- amino)phenyl]sulfonyl}a- mino)propyl]-4,5-dihydro-2H-pyra- zolo[3,4-f]isoquinoline-3-carboxylic acid trifluoroacetate | 176 | 200 |
| 277 | 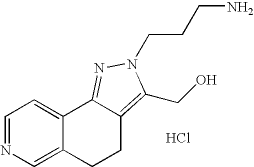 | [2-(3-amino- propyl)-4,5-dihydro-2H-py- razolo[3,4-f]iso- quinolin-3-yl]methanol hydrochloride | 186 | |
| 278 | 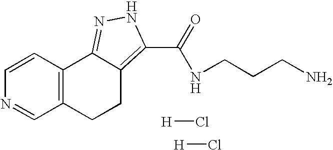 | N-(3-amino- propyl)-4,5-dihydro-2H-py- razolo[3,4-f]isoquinoline-3 carboxamide dihydrochloride | 189 | 200 |
| 279 | 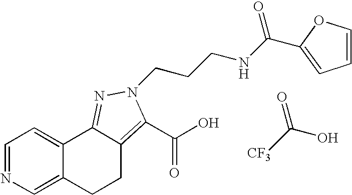 | 2-[3-(2-furoylamino)propyl]-4,5-di- hydro-2H-pyrazolo[3,4-f]iso- quinoline-3-carboxylic acid trifluoroacetate | 190 | 200 |
| 280 | 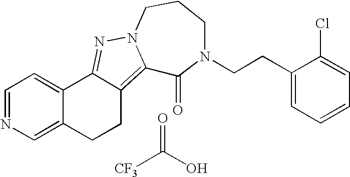 | 8-[2-(2-chloro- phenyl)ethyl]-5,6,8,9,10,11-hexa- hydro-7H-[1,4]diaze- pino[1′,2′:1,5]pyrazolo[3,4-f]iso- quinolin-7-one trifluoroacetate | 190 | 200 |
| 281 | 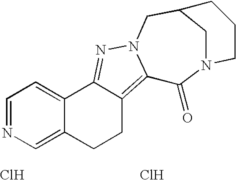 | 5,6,10,11,12,13-hexa- hydro-7H,9H-8,12-metha- no[1,4]diazonino[1′,2′:1,5]py- razolo[3,4-f]isoquinolin-7-one dihydrochloride | 191 | |
| 282 | 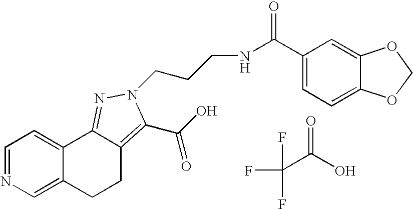 | 2-{3-[(1,3-benzodioxol-5-ylcarbo- nyl)amino]propyl}-4,5-di- hydro-2H-pyrazolo[3,4-f]iso- quinoline-3-carboxylic acid trifluoroacetate | 194 | 200 |
| 283 | 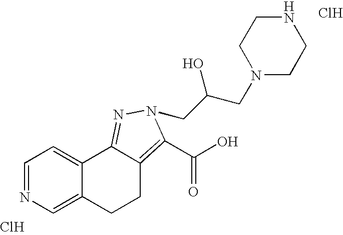 | 2-(2-hydroxy-3-piperazin-1-ylpro- pyl)-4,5-dihydro-2H-py- razolo[3,4-f]iso- quinoline-3-carboxylic acid dihydrochloride | 198 | |
| 284 | 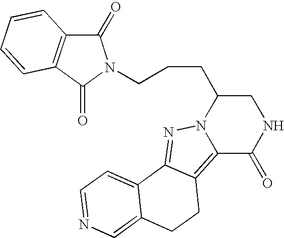 | 2-[3-(7-oxo-5,6,7,8,9,10-hexa- hydropyrazino[1′,2′:1,5]pyra- zolo[3,4-f]isoquinolin-10-yl)pro- pyl]-1H-isoindole-1,3(2H)-dione | 200 | |
| 285 | 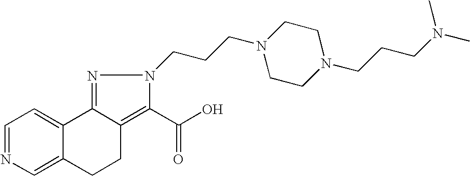 | 2-(3-{4-[3-(di- methylamino)propyl]piperazin 1-yl}propyl)-4,5-dihydro-2H-py- razolo[3,4-f]iso- quinoline-3-carboxylic acid | 200 | |
| 286 | 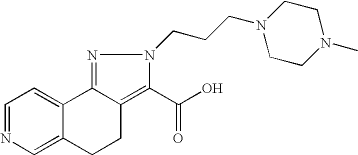 | 2-[3-(4-methylpiperazin-1-yl)pro- pyl]-4,5-dihydro-2H-py- razolo[3,4-f]iso- quinoline-3-carboxylic acid | 200 | |
| 287 | 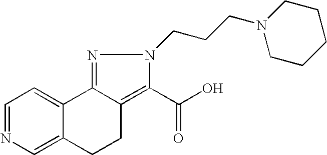 | 2-(3-piperidin-1-ylpropyl)-4,5-di- hydro-2H-pyrazolo[3,4-f]iso- quinoline-3-carboxylic acid | 200 | |
| 288 | 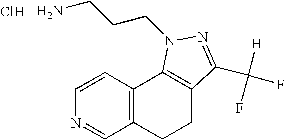 | 3-[3-(difluoromethyl)-4,5-di- hydro-1H-pyrazolo[3,4-f]iso- quinolin-1-yl]propan-1-amine hydrochloride | 200 | |
| 289 | 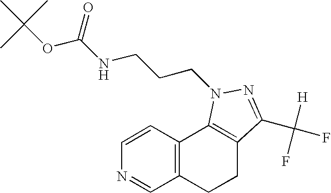 | tert-butyl 3-[3-(di- fluoromethyl)-4,5-di- hydro-1H-pyrazolo[3,4-f]iso- quinolin-1-yl]propylcarbamate | 200 | |
| 290 | 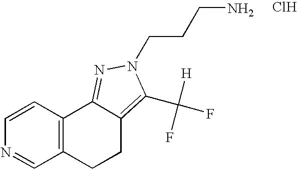 | 3-[3-(difluoromethyl)-4,5-di- hydro-2H-pyrazolo[3,4-f]iso- quinolin-2-yl]propan-1-amine hydrochloride | 200 | |
| 291 | 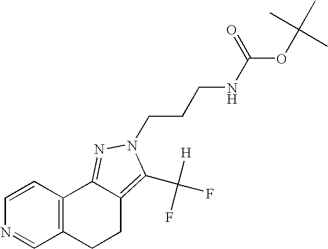 | tert-butyl 3-[3-(di- fluoromethyl)-4,5-di- hydro-2H-pyrazolo[3,4-f]iso- quinolin-2-yl]propylcarbamate | 200 | |
| 292 | 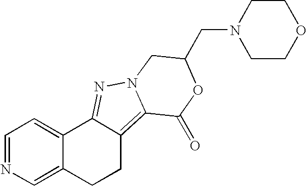 | 9-(morpho- lin-4-ylmethyl)-5,6,9,10-tetra- hydro-7H-[1,4]oxa- zino[4′,3′:1,5]pyrazolo[3,4-f]iso- quinolin-7-one | 200 | |
| 293 | 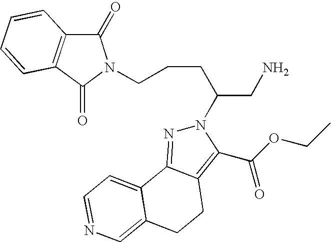 | ethyl 2-[1-(aminomethyl)-4-(1,3-di- oxo-1,3-dihydro-2H-isoindol-2-yl)bu- tyl]-4,5-dihydro-2H-py- razolo[3,4-f]iso- quinoline-3-carboxylate | 200 | |
| 294 | 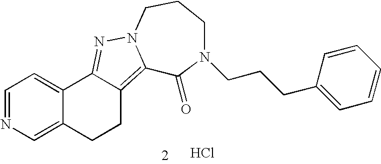 | 8-(3-phenyl- propyl)-5,6,8,9,10,11-hexa- hydro-7H-[1,4]diaze- pino[1′,2′:1,5]pyrazolo[3,4-f]iso- quinolin-7-one hydrochloride | 200 | 200 |
| 295 | 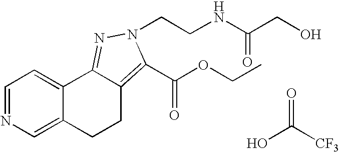 | ethyl 2-[2-(glycoloyl- amino)ethyl]4,5-di- hydro-2H-pyrazolo[3,4-f]iso- quinoline-3-carboxylate trifluoroacetate | 200 | 200 |
| 296 | 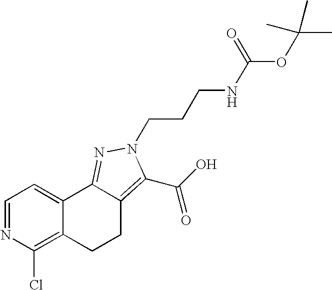 | 2-{3-[(tert- butoxycarbonyl)amino]propyl}-6 chloro-4,5-dihydro-2H-py- razolo[3,4-f]iso- quinoline-3-carboxylic acid | 200 | |
| 297 | 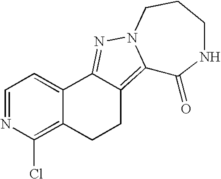 | 4-chloro-5,6,8,9,10,11-hexa- hydro-7H-[1,4]diaze- pino[1′,2′:1,5]pyrazolo[3,4-f]iso- quinolin-7-one | 200 | |
| 298 | 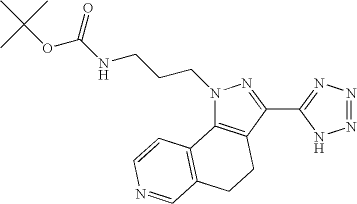 | tert-butyl 3-[3-(1H-tetra- azol-5-yl)-4,5-di- hydro-1H-pyrazolo[3,4-f]iso- quinolin-1-yl]propylcarbamate | 200 | |
| 299 | 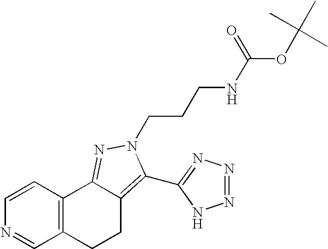 | tert-butyl 3-[3-(1H-tetra- azol-5-yl)-4,5-di- hydro-2H-pyrazolo[3,4-f]iso- quinolin-2-yl]propylcarbamate | 200 | |
| 300 | 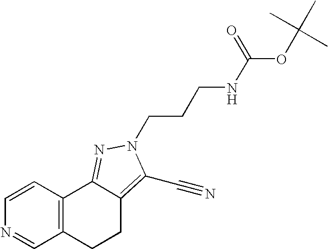 | tert-butyl 3-(3-cyano-4,5-di- hydro-2H-pyrazolo[3,4-f]iso- quinolin-2-yl)propylcarbamate | 200 | |
| 301 | 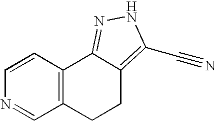 | 4,5-dihydro-2H-pyrazolo[3,4-f]iso- quinoline-3-carbonitrile | 200 | |
| 302 | 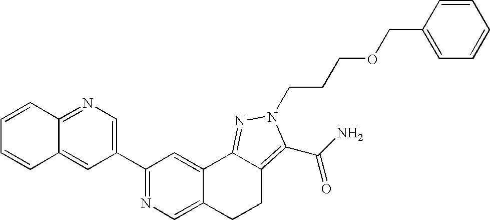 | 2-[3-(benzyloxy)propyl]-8-quino- lin-3-yl-4,5-dihydro-2H-py- razolo[3,4-f]iso- quinoline-3-carboxamide | 200 | |
| 303 | 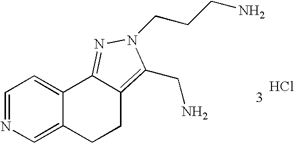 | 3-[3-(amino- methyl)-4,5-dihydro-2H-py- razolo[3,4-f]isoquinolin-2-yl]pro- pan-1-amine hydrochloride | 200 | |
| 304 | 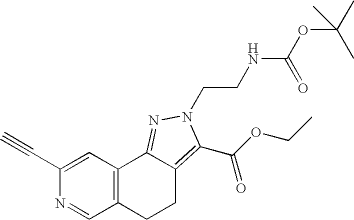 | ethyl 2-{2-[(tert- butoxycarbonyl)amino]ethyl}-8-eth- ynyl-4,5-dihydro-2H-py- razolo[3,4-f]isoquinoline-3-car- boxylate | 200 | |
| 305 | 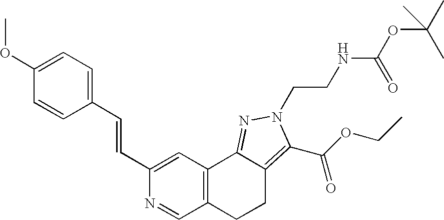 | ethyl 2-{2-[(tert- butoxycarbonyl)amino]eth- yl}-8-[(E)-2-(4-methoxy- phenyl)ethenyl]-4,5-di- hydro-2H-pyrazolo[3,4-f]iso- quinoline-3-carboxylate | 200 | |
| 306 | 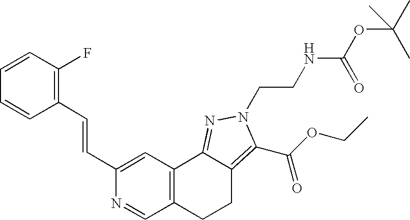 | ethyl 2-{2-[(tert- butoxycarbonyl)amino]eth- yl}-8-[(E)-2-(2-fluoro- phenyl)ethenyl]-4,5-di- hydro-2H-pyrazolo[3,4-f]iso- quinoline-3-carboxylate | 200 | |
| 307 | 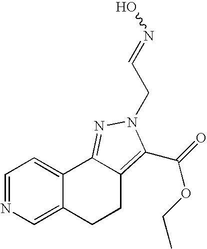 | ethyl 2-[(2E)-2-(hy- droxyimino)ethyl]-4,5-di- hydro-2H-pyrazolo[3,4-f]iso- quinoline-3-carboxylate | 200 | |
| 308 | 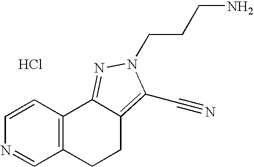 | 2-(3-amino- propyl)-4,5-dihydro-2H-py- razolo[3,4-f]isoquinoline-3 carbonitrile hydrochloride | 200 | |
| 309 | 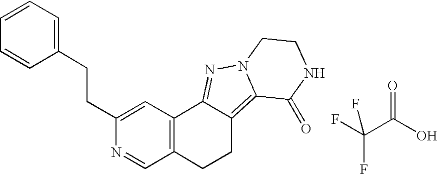 | 2-(2-phenylethyl)-5,6,9,10-tetra- hydropyrazino[1′,2′:1,5]pyra- zolo[3,4-f]isoquinolin-7(8H)-one trifluoroacetate | 200 | |
| 310 | 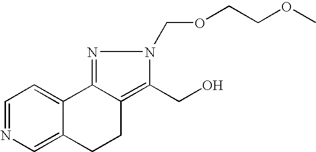 | {2-[(2-methoxy- ethoxy)methyl]-4,5-di- hydro-2H-pyrazolo[3,4-f]iso- quinolin-3-yl}methanol | 200 | |
| 311 | 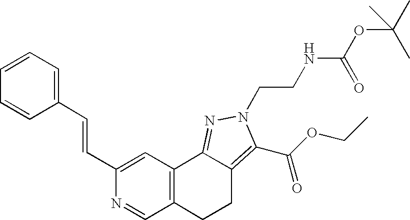 | ethyl 2-{2-[(tert- butoxycarbonyl)amino]eth- yl}-8-[(E)-2-phenyl- ethenyl]-4,5-di- hydro-2H-pyrazolo[3,4-f]iso- quinoline-3-carboxylate | 200 | |
| 312 | 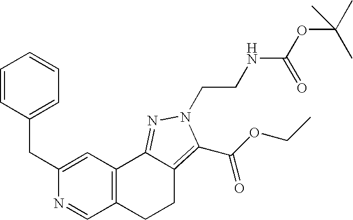 | ethyl 8-benzyl-2-{2-[(tert- butoxycarbonyl)amino]ethyl}-4,5 dihydro-2H-pyrazolo[3,4-f]iso- quinoline-3-carboxylate | 200 | |
| 313 | 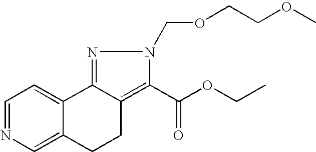 | ethyl 2-[(2-methoxy- ethoxy)methyl]-4,5-di- hydro-2H-pyrazolo[3,4-f]iso- quinoline-3-carboxylate | 200 | |
| 314 | 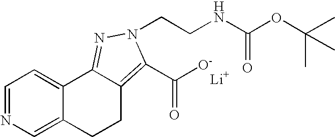 | lithium 2-{2-[(tert-butoxy- carbonyl)amino]ethyl}-4,5 dihydro-2H-pyrazolo[3,4-f]iso- quinoline-3-carboxylate | 200 | |
| 315 | 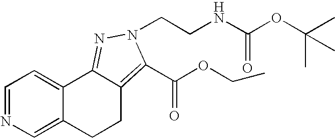 | ethyl 2-{2-[(tert- butoxycarbonyl)amino]ethyl}-4,5 dihydro-2H-pyrazolo[3,4-f]iso- quinoline-3-carboxylate | 200 | |
| 316 | 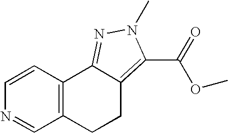 | methyl 2-methyl-4,5-dihydro-2H pyrazolo[3,4-f]isoquinoline-3-car- boxylate | 200 | 200 |
| 317 | 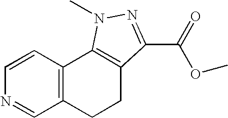 | methyl 1-methyl-4,5-dihydro-1H pyrazolo[3,4-f]isoquinoline-3-car- boxylate | 200 | 200 |
| 318 | 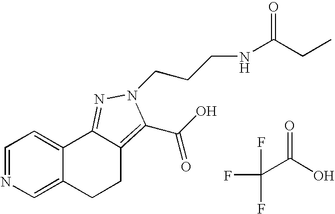 | 2-[3-(propionylamino)propyl]-4,5 dihydro-2H-pyrazolo[3,4-f]iso- quinoline-3-carboxylic acid trifluoroacetate | 200 | 200 |
| 319 | 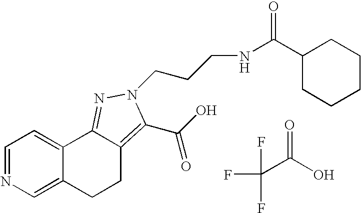 | 2-{3-[(cyclo- hexylcarbonyl)amino]pro- pyl}-4,5-dihydro-2H-py- razolo[3,4-f]iso- quinoline-3-carboxylic acid trifluoroacetate | 200 | 200 |
| 320 | 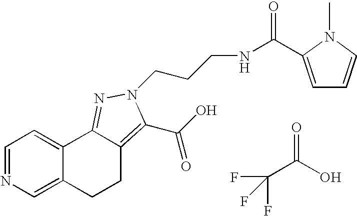 | 2-(3-{[(1-methyl-1H-pyrrol-2-yl)car- bonyl]amino}propyl)-4,5-di- hydro-2H-pyrazolo[3,4-f]iso- quinoline-3-carboxylic acid trifuoroacetate | 200 | 200 |
| 321 | 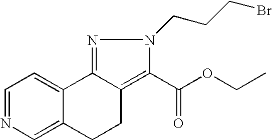 | ethyl 2-(3-bromopropyl)-4,5-di- hydro-2H-pyrazolo[3,4-f]iso- quinoline-3-carboxylate | 200 | |
| 322 | 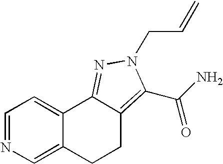 | 2-allyl-4,5-dihydro-2H-py- razolo[3,4-f]isoquinoline-3-car- boxamide | 200 | 200 |
| 323 | 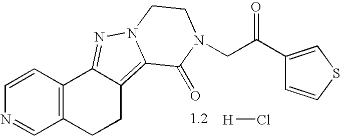 | 8-(2-oxo-2-thien-3-yleth- yl)-5,6,9,10-tetra- hydropyrazino[1′,2′,1,5]pyra- zolo[3,4-f]isoquinolin-7(8H)-one hydrochloride | 200 | |
| 324 | 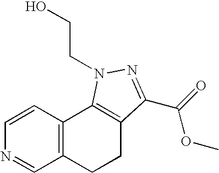 | methyl 1-(2-hydroxyethyl)-4,5-di- hydro-1H-pyrazolo[3,4-f]iso- quinoline-3-carboxylate | 200 | 200 |
| 325 | 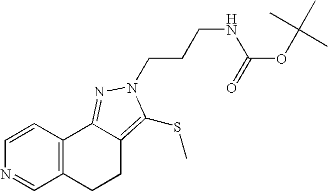 | tert-butyl 3-[3-(methylthio)-4,5-di- hydro-2H-pyrazolo[3,4-f]iso- quinolin-2-yl]propyl- carbamate | 200 | 200 |
| 326 | 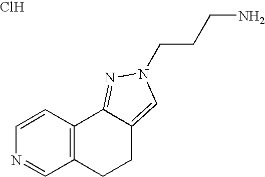 | 3-(4,5-dihydro-2H-pyrazolo[3,4-f]iso- quinolin-2-yl)propan-1-amine hydrochloride | 200 | 200 |
| 327 | 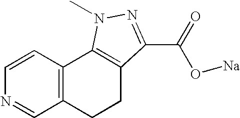 | | 200 | 200 |
| 328 | 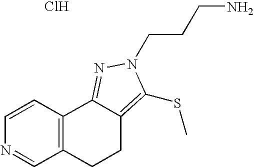 | 3-[3-(methylthio)-4,5-dihydro-2H pyrazolo[3,4-f]isoquinolin-2-yl]pro- pan-1-amine hydrochloride | 200 | 200 |
| 329 | 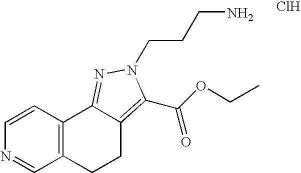 | ethyl 2-(3-aminopropyl)-4,5-di- hydro-2H-pyrazolo[3,4-f]iso- quinoline-3-carboxylate hydrochloride | 200 | 200 |
| 330 | 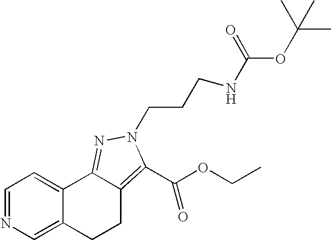 | ethyl 2-{3-[(tert- butoxycarbonyl)a- mino]propyl}-4,5-di- hydro-2H-pyrazolo[3,4-f]iso- quinoline-3-carboxylate | 200 | 200 |
| 331 | 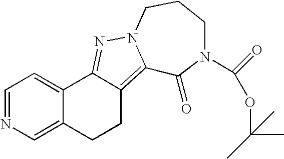 | tert-butyl 7-oxo-6,7,10,11-tetra- hydro-5H-[1,4]diaze- pino[1′,2′:1,5]pyrazolo[3,4-f]iso- quinoline-8(9H)-carboxylate | 200 | 200 |
| 332 | 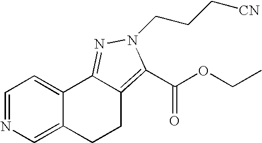 | ethyl 2-(3-cyanopropyl)-4,5-di- hydro-2H-pyrazolo[3,4-f]iso- quinoline-3-carboxylate | 200 | |
| 333 | 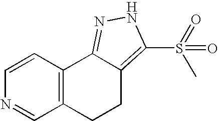 | 3-(methyl- sulfonyl)-4,5-dihydro-2H-py- razolo[3,4-f]isoquinoline | 200 | 200 |
| 334 | 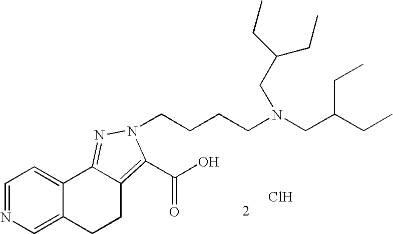 | 2-{4-[bis(2-ethyl- butyl)amino]butyl}-4,5-di- hydro-2H-pyrazolo[3,4-f]iso- quinoline-3-carboxylic acid hydrochloride | 200 | |
| 335 | 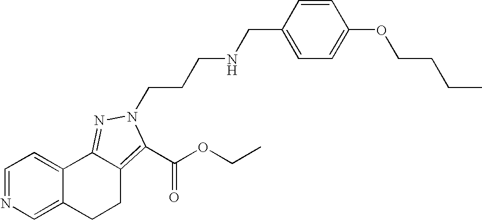 | ethyl 2-{3-[(4-butoxy- benzyl)amino]propyl}-4,5-di- hydro-2H-pyrazolo[3,4-f]iso- quinoline-3-carboxylate | 200 | |
| 336 | 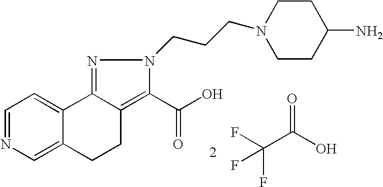 | 2-[3-(4-aminopiperidin-1-yl)pro- pyl]-4,5-dihydro-2H-py- razolo[3,4-f]iso- quinoline-3-carboxylic acid trifluoroacetate | 200 | |
| 337 | 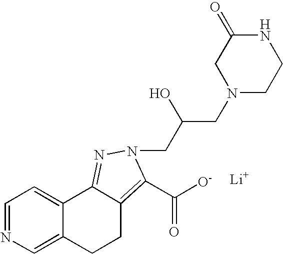 | lithium 2-[2-hydroxy-3-(3-oxo- piperazin-1-yl)propyl]-4,5-di- hydro-2H-pyrazolo[3,4-f]iso- quinoline-3-carboxylate | 200 | |
| 338 | 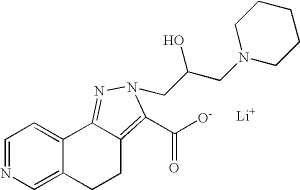 | lithium 2-(2-hy- droxy-3-piperidin-1-ylpro- pyl)-4,5-dihydro-2H-py- razolo[3,4-f]iso- quinoline-3-carboxylate | 200 | |
| 339 | 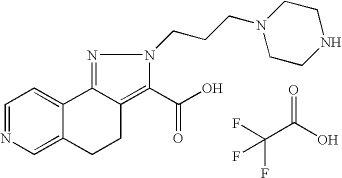 | 2-(3-piperazin-1-ylpropyl)-4,5-di- hydro-2H-pyrazolo[3,4-f]iso- quinoline-3-carboxylic acid trifluoroacetate | 200 | |
| 340 | 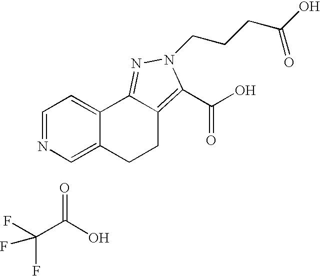 | 2-(3-carboxypropyl)-4,5-dihydro 2H-pyrazolo[3,4-f]isoquinoline-3 carboxylic acid trifluoroacetate | 200 | |
| 341 | 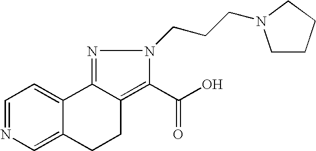 | 2-(3-pyrrolidin-1-ylpropyl)-4,5-di- hydro-2H-pyrazolo[3,4-f]iso- quinoline-3-carboxylic acid | 200 | |
| 342 | 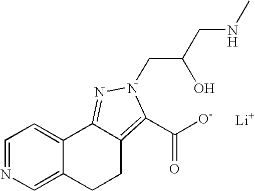 | lithium 2-[2-hydroxy-3-(methyl- amino)propyl]-4,5-di- hydro-2H-pyrazolo[3,4-f]iso- quinoline-3-carboxylate | 200 | |
| 343 | 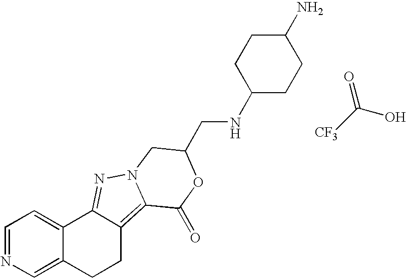 | 9-{[(4-amino- cyclohexyl)amino]meth- yl}-5,6,9,10-tetra- hydro-7H-[1,4]oxa- zino[4′,3′:1,5]pyrazolo[3,4-f]iso- quinolin-7-one trifluoroacetate | 200 | |
| 344 | 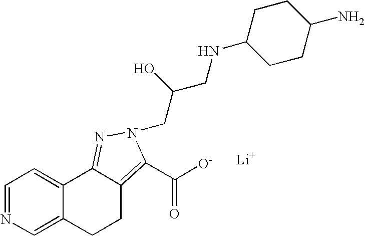 | lithium 2-{3-[(4-amino- cyclohexyl)amino]-2-hydroxy- propyl}-4,5-dihydro-2H-py- razolo[3,4-f]iso- quinoline-3-carboxylate | 200 | |
| 345 | 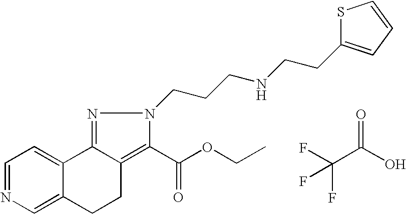 | ethyl2-{3-[(2-thien-2-yleth- yl)amino]propyl}-4,5-di- hydro-2H-pyrazolo[3,4-f]iso- quinoline-3-carboxylate trifluoroacetate | 200 | |
| 346 | 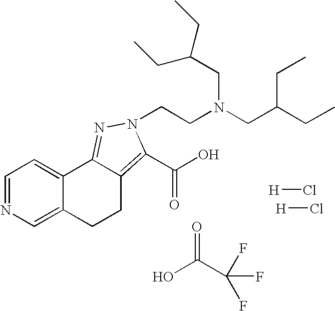 | 2-{2-[bis(2-ethyl- butyl)amino]ethyl}-4,5-di- hydro-2H-pyrazolo[3,4-f]iso- quinoline-3-carboxylic acid dihydrochloride | 200 | |
| 347 | 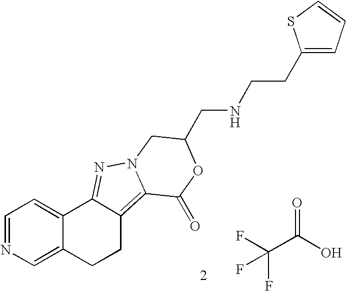 | 9-{[(2-thien-2-yleth- yl)amino]methyl}-5,6,9,10-tetra- hydro-7H-[1,4]oxa- zino[4′,3′:1,5]pyrazolo[3,4-f]iso- quinolin-7-one trifluoroacetate | 200 | |
| 348 | 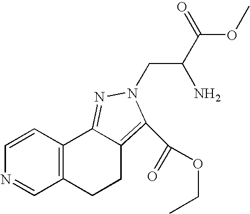 | | 200 | |
| 349 | 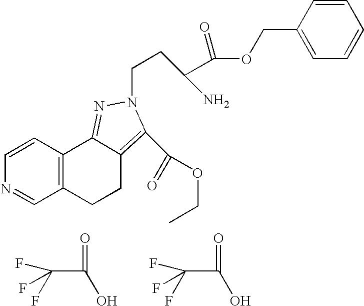 | ethyl 2-[3-amino-4-(benzyl- oxy)-4-oxo- butyl]-4,5-dihydro-2H-py- razolo[3,4-f]iso- quinoline-3-carboxylate bis(trifluoroacetate) | 200 | |
| 350 | 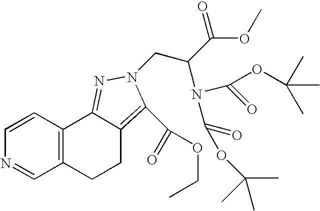 | | 200 | |
| 351 | 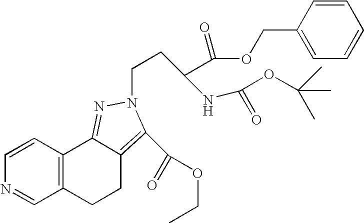 | ethyl 2-{4-(benzyloxy)-3-[(tert- butoxycarbonyl)amino]-4-oxo- butyl}-4,5-dihydro-2H-py- razolo[3,4-f]isoquinoline-3-car- boxylate | 200 | |
| 352 | 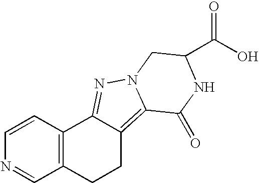 | 7-oxo-5,6,7,8,9,10-hexa- hydropyrazino[1′,2′:1,5]py- razolo[3,4-f]isoquinoline-9-car- boxylic acid | 200 | |
| 353 | 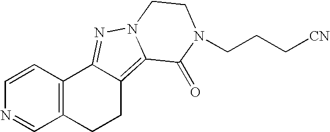 | 4-(7-oxo-6,7,9,10-tetra- hydropyrazino[1′,2′:1,5]pyra- zolo[3,4-f]isoquinolin-8(5H)-yl)bu- tanenitrile | 200 | |
| 354 | 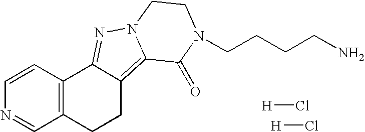 | 8-(4-aminobutyl)-5,6,9,10-tetra- hydropyrazino[1′,2′:1,5]pyra- zolo[3,4-f]isoquinolin-7(8H)-one dihydrochloride | 200 | |
| 355 | 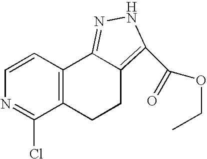 | ethyl 6-chloro-4,5-dihydro-2H-py- razolo[3,4-f]isoquinoline-3-car- boxylate | 200 | |
| 356 | 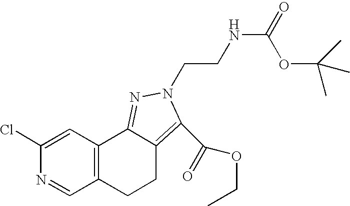 | ethyl 2-{2-[(tert- butoxycarbonyl)amino]eth- yl}-8-chloro-4,5-di- hydro-2H-pyrazolo[3,4-f]iso- quinoline-3-carboxylate | 200 | |
| 357 | 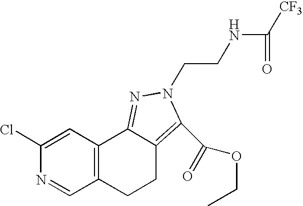 | ethyl 8-chloro-2-{2-[(tri- fluoroacetyl)amino]ethyl}-4,5 dihydro-2H-pyrazolo[3,4-f]iso- quinoline-3-carboxylate | 200 | |
| 358 | 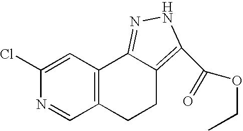 | ethyl 8-chloro-4,5-dihydro-2H-py- razolo[3,4-f]iso- quinoline-3-carboxylate | 200 | |
| 359 | 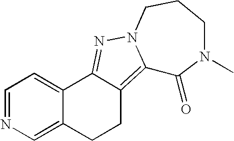 | 8-methyl-5,6,8,9,10,11-hexa- hydro-7H-[1,4]diaze- pino[1′,2′:1,5]pyrazolo[3,4-f]iso- quinolin-7-one | 200 | |
| 360 | 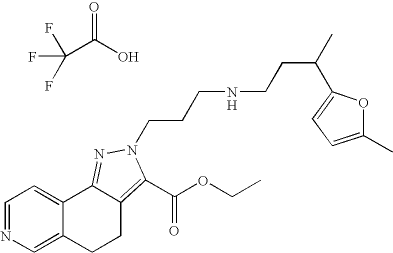 | ethyl 2-(3-{[3-(5-methyl-2-furyl)bu- tyl]amino}propyl)-4,5-di- hydro-2H-pyrazolo[3,4-f]iso- quinoline-3-carboxylate trifluoroacetate | 200 | |
| 361 | 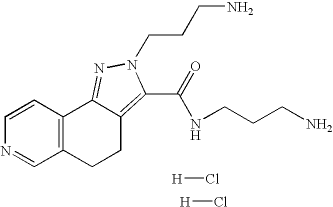 | N,2-bis(3-aminopropyl)-4,5-di- hydro-2H-pyrazolo[3,4-f]iso- quinoline-3-carboxamide dihydrochloride | 200 | 200 |
| 362 | 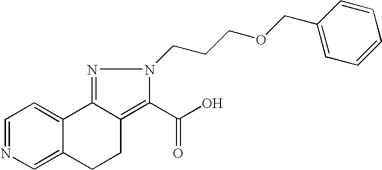 | 2-[3-(benzyloxy)propyl]-4,5-di- hydro-2H-pyrazolo[3,4-f]iso- quinoline-3-carboxylic acid | 200 | |
| 363 | 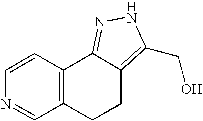 | 4,5-dihydro-2H-pyrazolo[3,4-f]iso- quinolin-3-ylmethanol | 200 | |
| 364 | 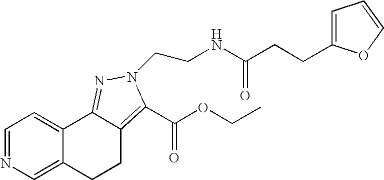 | ethyl 2-(2-{[3-(2-furyl)propa- noyl]amino}ethyl)-4,5 dihydro-2H-pyrazolo[3,4-f]iso- quinoline-3-carboxylate | 200 | |
| 365 | 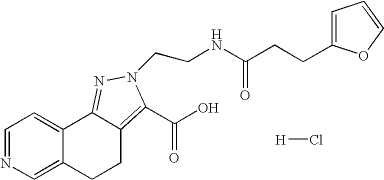 | 2-(2-{[3-(2-furyl)propa- noyl]amino}ethyl)-4,5 dihydro-2H-pyrazolo[3,4-f]iso- quinoline-3-carboxylic acid hydrochloride | 200 | |
| 366 | 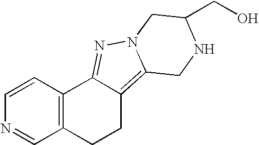 | 5,6,7,8,9,10-hexa- hydropyrazino[1′,2′:1,5]pyra- zolo[3,4-f]isoquinolin-9-ylmethanol | 200 | |
| 367 | 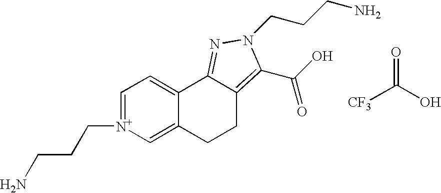 | For multi-component charged structures, a total zero charge is required! | 200 | 200 |
| 368 | 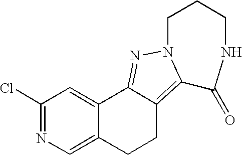 | 2-chloro-5,6,8,9,10,11-hexa- hydro-7H-[1,4]diaze- pino[1′,2′:1,5]pyrazolo[3,4-f]iso- quinolin-7-one | 200 | |
| 369 | 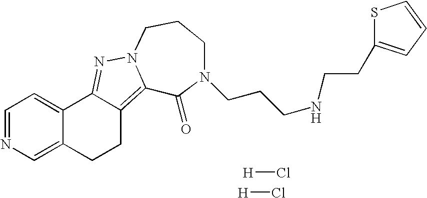 | 8-{3-[(2-thien-2-yleth- yl)amino]propyl}-5,6,8,9,10,11-hexa- hydro-7H-[1,4]diaze- pino[1′,2′:1,5]pyrazolo[3,4-f]iso- quinolin-7-one dihydrochloride | 200 | |
| 370 | 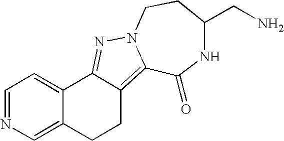 | 9-(aminomethyl)-5,6,8,9,10,11-hexa- hydro-7H-[1,4]diaze- pino[1′,2′:1,5]pyrazolo[3,4-f]iso- quinolin-7-one | 200 | |
| 371 | 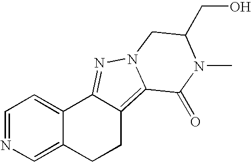 | 9-(hydroxy- methyl)-8-methyl-5,6,9,10-tetra- hydropyrazino[1′,2′:1,5]pyra- zolo[3,4-f]isoquinolin-7(8H)-one | 200 | |
| 372 | 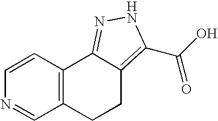 | 4,5-dihydro-2H-pyrazolo[3,4-f]iso- quinoline-3-carboxylic acid | | |
| 373 | 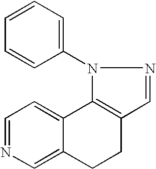 | | | |
| 374 | 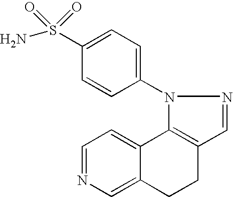 | | | |
| 375 | 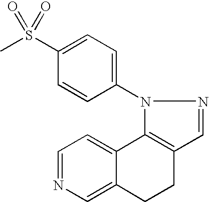 | | | |
| 376 | 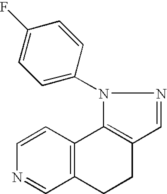 | | | |
| 377 | 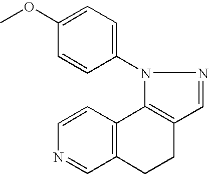 | | | |
| 378 | 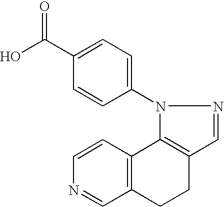 | | | |
| 379 | 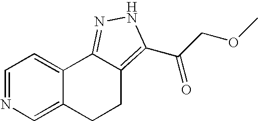 | 1-(4,5-dihydro-2H-pyrazolo[3,4-f]iso- quinolin-3-yl)-2-methoxy- ethanone | | |
| 380 | 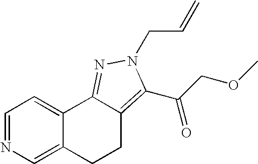 | ethyl 2-allyl-4,5-dihydro-2H-py- razolo[3,4-f]isoquinoline-3-car- boxylate | | |
| 381 | 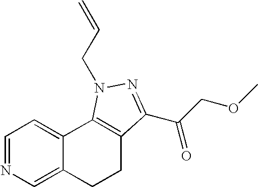 | ethyl 1-allyl-4,5-dihydro-1H-py- razolo[3,4-f]isoquinoline-3-car- boxylate | | |
| 382 | 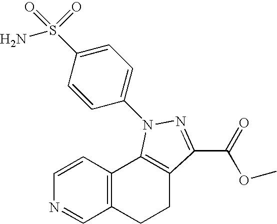 | | | |
| 383 | 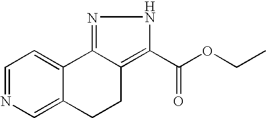 | ethyl 4,5-dihydro-2H-py- razolo[3,4-f]isoquinoline-3-car- boxylate | | |
| 384 | 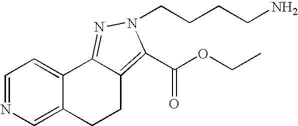 | ethyl 2-(4-aminobutyl)-4,5-di- hydro-2H-pyrazolo[3,4-f]iso- quinoline-3-carboxylate | | |
| 385 | 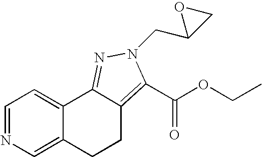 | ethyl 2-(oxiran-2-ylmethyl)-4,5-di- hydro-2H-pyrazolo[3,4-f]iso- quinoline-3-carboxylate | | |
| 386 | 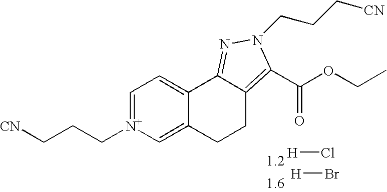 | 2,7-bis(3-cyanopropyl)-3-(ethoxy- carbonyl)-4,5-dihydro-2H-py- razolo[3,4-f]isoquinolin-7-ium | | |
| 387 | 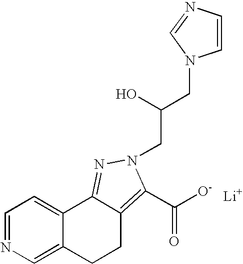 | lithium 2-[2-hydroxy-3-(1H-imi- dazol-1-yl)propyl]-4,5-di- hydro-2H-pyrazolo[3,4-f]iso- quinoline-3-carboxylate | | |
| 388 | 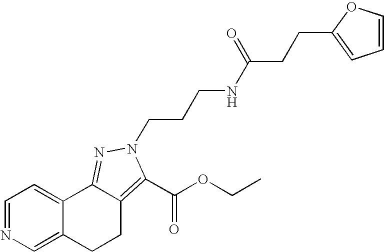 | ethyl 2-(3-{[3-(2-furyl)pro- panoyl]amino}propyl)-4,5-di- hydro-2H-pyrazolo[3,4-f]iso- quinoline-3-carboxylate | | |
| 389 | 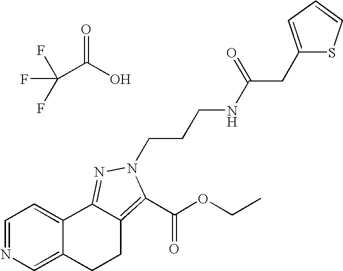 | ethyl 2-{3-[(thien-2-ylace- tyl)amino]propyl}-4,5-di- hydro-2H-pyrazolo[3,4-f]iso- quinoline-3-carboxylate trifluoroacetate | | |
| 390 | 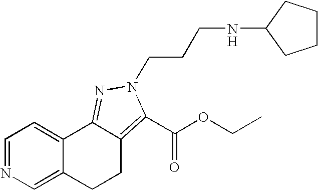 | ethyl 2-[3-(cyclo- pentylamino)propyl]-4,5-di- hydro-2H-pyrazolo[3,4-f]iso- quinoline-3-carboxylate | | |
| 391 | 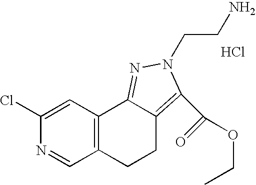 | ethyl 2-(2-amino- ethyl)-8-chloro-4,5-di- hydro-2H-pyrazolo[3,4-f]iso- quinoline-3-carboxylate hydrochloride | | |
| 392 | 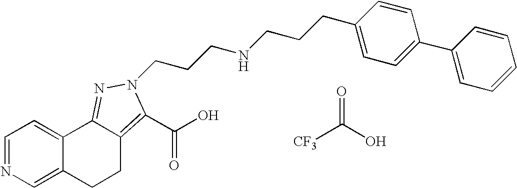 | 2-(3-{[3-(1,1′-bi- phenyl-4-yl)pro- pyl]amino}propyl)-4,5-di- hydro-2H-pyrazolo[3,4-f]iso- quinoline-3-carboxylic acid trifluoroacetate | | |
| 393 | 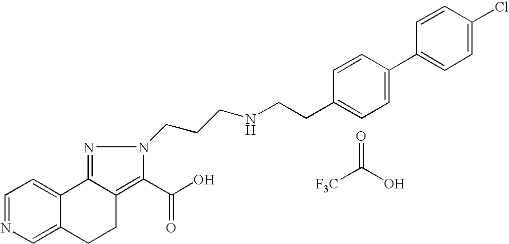 | 2-(3-{[2-(4′-chloro-1,1′-bi- phenyl-4-yl)eth- yl]amino}propyl)-4,5-di- hydro-2H-pyrazolo[3,4-f]iso- quinoline-3-carboxylic acid trifluoroacetate | | |
| 394 | 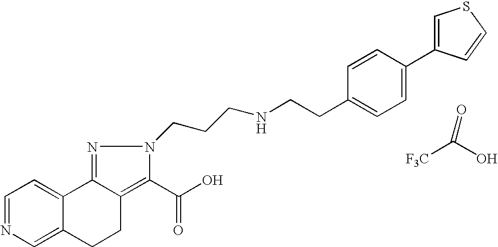 | 2-(3-{[2-(4-thien-3-ylphen- yl)ethyl]amino}propyl)-4,5 dihydro-2H-pyrazolo[3,4-f]iso- quinoline-3-carboxylic acid trilfuoroacetate | | |
| 395 | 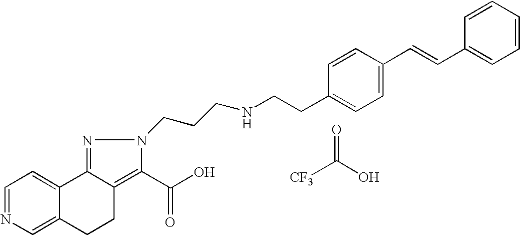 | 2-{3-[(2-{4-[(E)-2-phenyl- ethenyl]phenyl}ethyl)a- mino]propyl}-4,5-dihydro-2H-py- razolo[3,4-f]isoquinoline-3-carboxy- lic acid trifluoroacetate | | |
| 396 | 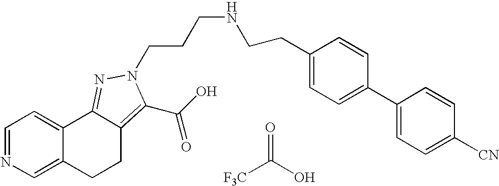 | 2-(3-{[2-(4′-cy- ano-1,1′-biphenyl-4-yl)eth- yl]amino}propyl)-4,5-di- hydro-2H-pyrazolo[3,4-f]iso- quinoline-3-carboxylic acid trifluoroacetate | | |
| 397 | 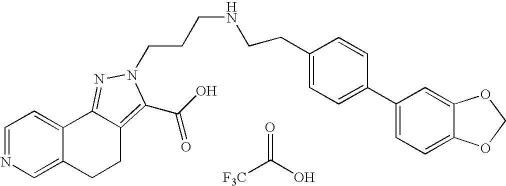 | 2-[3-({2-[4-(1,3-benzo- dioxol-5-yl)phen- yl]ethyl}amino)propyl]-4,5-di- hydro-2H-pyrazolo[3,4-f]iso- quinoline-3-carboxylic acid trifluoroacetate | | |
| 398 | 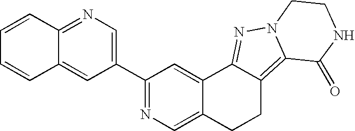 | 2-quinolin-3-yl-5,6,9,10-tetra- hydropyrazino[1′,2′:1,5]pyra- zolo[3,4-f]isoquinolin-7(8H)-one | | |
| 399 | 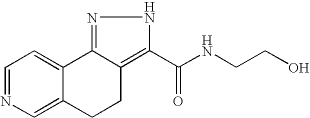 | N-(2-hydroxy- ethyl)-4,5-dihydro-2H-py- razolo[3,4-f]isoquinoline-3 carboxamide | | |
| 400 | 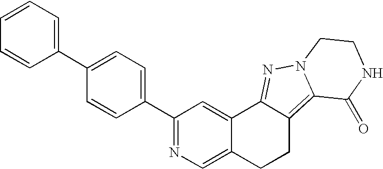 | 2-(1,1′-biphenyl-4-yl)-5,6,9,10-tetra- hydropyrazino[1′,2′:1,5]pyra- zolo[3,4-f]isoquinolin-7(8H)-one | | |
| 401 | 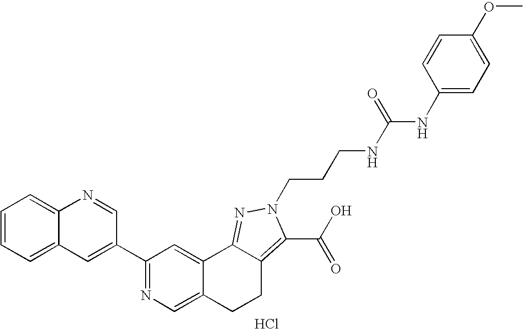 | 2-[3-({[(4-methoxy- phenyl)amino]carbonyl}a- mino)propyl]-8-quinolin-3-yl-4,5-di- hydro-2H-pyrazolo[3,4-f]iso- quinoline-3-carboxylic acid | | |
| 402 | 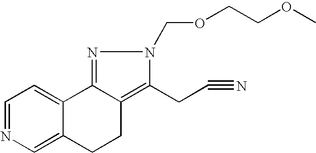 | {2-[(2-methoxy- ethoxy)methyl]-4,5-di- hydro-2H-pyrazolo[3,4-f]iso- quinolin-3-yl}acetonitrile | | |
| 403 | 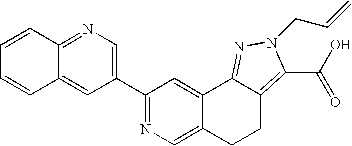 | 2-allyl-8-quinolin-3-yl-4,5-di- hydro-2H-pyrazolo[3,4-f]iso- quinoline-3-carboxylic acid | | |
| 404 | 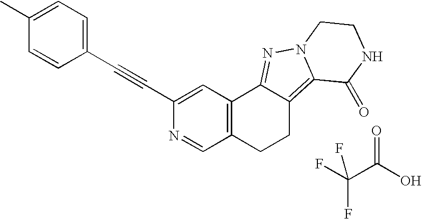 | 2-[(4-methyl- phenyl)ethynyl]-5,6,9,10-tetra- hydropyrazino[1′,2′:1,5]pyra- zolo[3,4-f]isoquinolin-7(8H)-one trifluoroacetate | | |
| 405 | 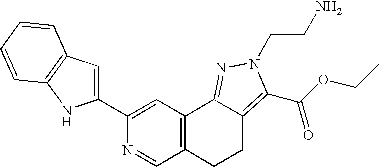 | ethyl 2-(2-aminoethyl)-8-(1H-in- dol-2-yl)-4,5-dihydro-2H-py- razolo[3,4-f]iso- quinoline-3-carboxylate | | |
| 406 | 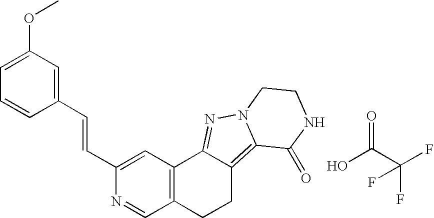 | 2-[(E)-2-(3-methoxy- phenyl)ethenyl]-5,6,9,10-tetra- hydropyrazino[1′,2′:1,5]pyra- zolo[3,4-f]isoquinolin-7(8H)-one trilfuoroacetate | | |
| 407 | 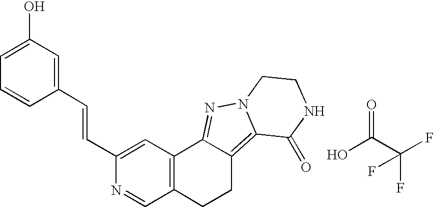 | 2-[(E)-2-(3-hydroxy- phenyl)ethenyl]-5,6,9,10 tetrahydropyrazino[1′,2′:1,5]pyra- zolo[3,4-f]isoquinolin-7(8H)-one trifluoroacetate | | |
| 408 | 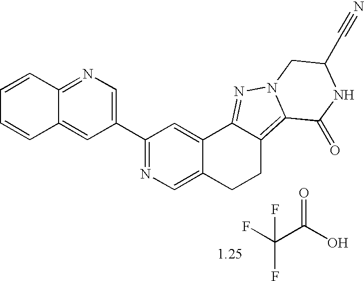 | 12593 or 7-oxo-2-quino- lin-3-yl-5,6,7,8,9,10-hexa- hydropyrazino[1′,2′:1,5]pyra- zolo[3,4-f]isoquinoline-9-carbo- nitrile trifluoroacetate | | |

^{a}The pyridylpyrazole compound may be shown with a solvent, such as, for example, trifluoroacetate, with which it can form a salt. Both the salt and acid forms of the pyridylpyrazole compound are included in the present invention.
^{b}Compound names generated by ACD/Name software.

TABLE 4
Pyrimidylpyrazole MK-2 inhibitors

| | | | MK-2 | NK-3 |
|---|---|---|---|---|
| | | | Avg. IC50 | Avg. IC50 |
| No. | Structure^{a} | Chemical Name^{b} | (uM) | (uM) |
| 1 | 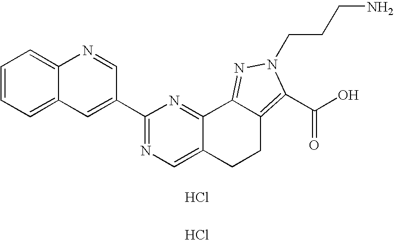 | 2-(3-aminopropyl)-8-quinolin-3-yl- 4,5-dihydro-2H-pyrazolo[4,3- h]quinazoline-3-carboxylic acid dihydrochloride | 0.0761 | 1.93 |
| 2 | 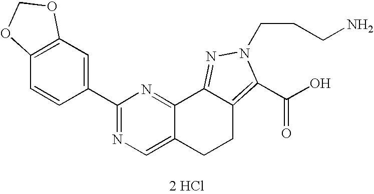 | 2-(3-aminopropyl)-8-(1,3- benzodioxol-5-yl)-4,5-dihydro-2H- pyrazolo[4,3-h]quinazoline-3- carboxylic acid hydrochloride | 0.295 | 4.61 |
| 3 | 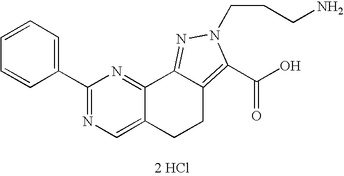 | 2-(3-aminopropyl)-8-phenyl-4,5- dihydro-2H-pyrazolo[4,3- h]quinazoline-3-carboxylic acid hydrochloride | 0.406 | 10.8 |
| 4 | 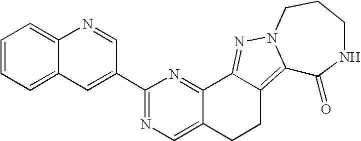 | 2-quinolin-3-yl-5,6,8,9,10,11- hexahydro-7H- [1,4]diazepino[1′,2′:1,5]pyrazolo[4, 4-h]quinazolin-7-one | 1.47 | 200 |
| 5 | 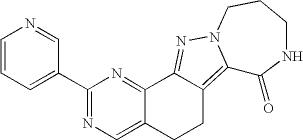 | 2-pyridin-3-yl-5,6,8,9,10,11- hexahydro-7H- [1,4]diazepino[1′,2′:1,5]pyrazolo[4, 3-h]quinazolin-7-one | 1.58 | 168 |
| 6 | 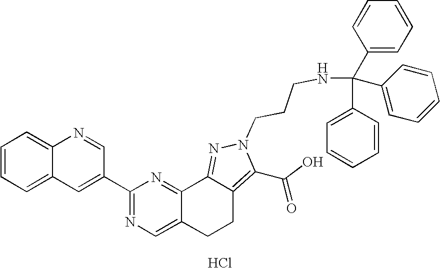 | 8-quinolin-3-yl-2-[3- (tritylamino)propyl]-4,5-dihydro-2H- pyrazolo[4,3-h]quinazoline-3- carboxylic acid hydrochloride | 2.16 | 154 |
| 7 | 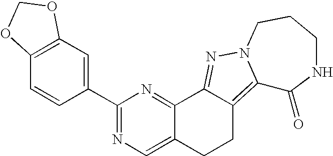 | 2-(1,3-benzodioxol-5-yl)- 5,6,8,9,10,11-hexahydro-7H- [1,4]diazepino[1′,2′:1,5]pyrazolo[4, 3-h]quinazolin-7-one | 3.14 | 200 |
| 8 | 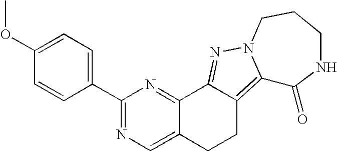 | 2-(4-methoxyphenyl)-5,6,8,9,10,11- hexahydro-7H- [1,4]diazepino[1′,2′:1,5]pyrazolo[4, 3-h]quinazolin-7-one | 4.52 | 200 |
| 9 | 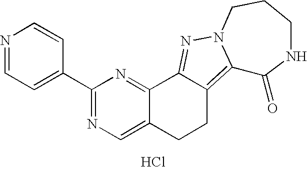 | 2-pyridin-4-yl-5,6,8,9,10,11- hexahydro-7H- [1,4]diazepino[1′,2′:1,5]pyrazolo[4, 3-h]quinazolin-7-one hydrochloride | 8.62 | 200 |
| 10 | 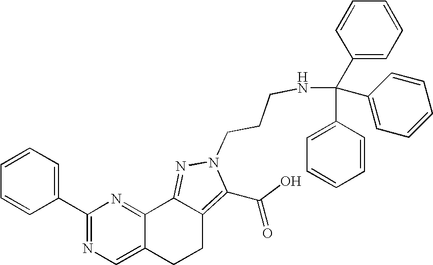 | 8-phenyl-2-[3-(tritylamino)propyl]- 4,5-dihydro-2H-pyrazolo[4,3- h]quinazoline-3-carboxylic acid | 52.2 | |
| 11 | 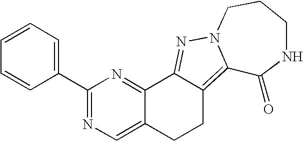 | 2-phenyl-5,6,8,9,10,11-hexahydro- 7H- [1,4]diazepino[1′,2′:1,5]pyrazolo[4, 3-h]quinazolin-7-one | 81.4 | 200 |
| 12 | 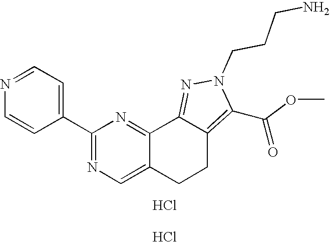 | methyl-2-(3-aminopropyl)-8-pyridin- 4-yl-4,5-dihydro-2H-pyrazolo[4,3- h]quinazoline-3-carboxylate dihydrochloride | 142 | |
| 13 | 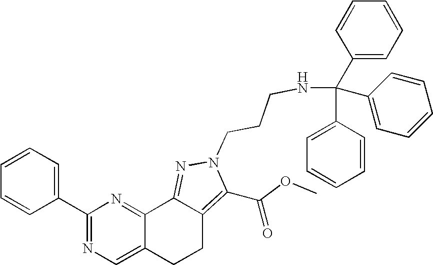 | methyl 8-phenyl-2-[3- (tritylamino)propyl]-4,5-dihydro-2H- pyrazolo[4,3-h]quinazoline-3- carboxylate | 200 | |
| 14 | 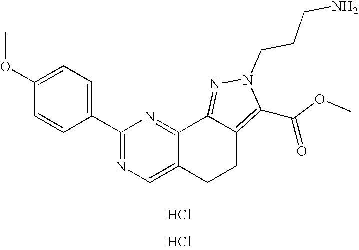 | methyl 2-(3-aminopropyl)-8-(4- methoxyphenyl)-4,5-dihydro-2H- pyrazolo[4,3-h]quinazoline-3- carboxylate dihydrochloride | 200 | |
| 15 | 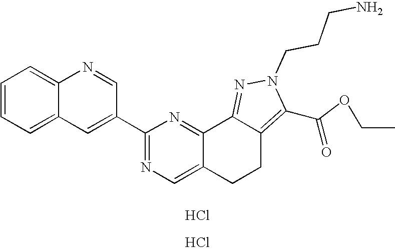 | ethyl 2-(3-aminopropyl)-8-quinolin- 3-yl-4,5-dihydro-2H-pyrazolo[4,3- h]quinazoline-3-carboxylate dihydrochloride | 200 | |

^{a}The pyrimidylpyrazole compound may be shown with a solvent, such as, for example, trifluoroacetate, with which it can form a salt. Both the salt and acid forms of the pyrimidylpyrazole compound are included in the present invention.
^{b}Compound names generated by ACD/Name software.

TABLE 5
Other cyclic pyrazole MK-2 inhibiting compounds

| | | | MK-2 | MK-3 |
|---|---|---|---|---|
| No. | Structure^{a} | Compound Name(s)^{b} | Avg. IC50 (uM) | Avg. IC50 (uM) |
| 1 | 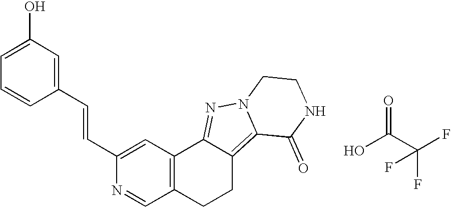 | 2-[(E)-2-(3- hydroxyphenyl)ethenyl]-5,6,9,10- tetrahydropyrazino[1′,2′:1,5]pyra- zolo[3,4-f]isoquinolin-7(8H)-one trifluoroacetate | 0.0211 | 3.62 |
| 2 | 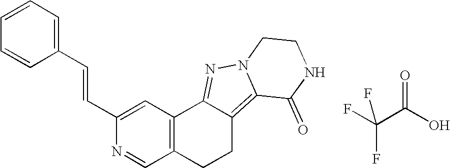 | 2-[(E)-2-phenylethenyl]-5,6,9,10- tetrahydropyrazino[1′,2′:1,5]pyra- zolo[3,4-f]isoquinolin-7(8H)-one trifluoroacetate | 0.147 | 11.5 |
| 3 | 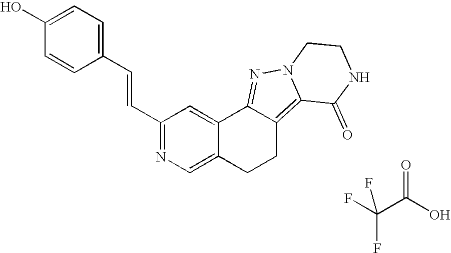 | 2-[(E)-2-(4- hydroxyphenyl)ethenyl]-5,6,9,10- tetrahydropyrazino[1′,2′:1,5]pyra- zolo[3,4-f]isoquinolin-7(8H)-one trifluoroacetate | 0.164 | 27.5 |
| 4 | 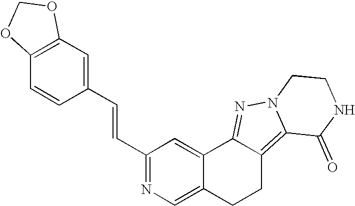 | 2-[(E)-2-(1,3-benzodioxol-5- yl)ethenyl]-5,6,9,10- tetrahydropyrazino[1′,2′:1,5]pyra- zolo[3,4-f]isoquinolin-7(8H)-one | 0.223 | >20.0 |
| 5 | 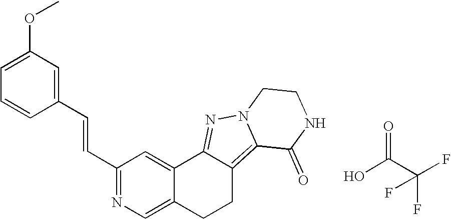 | 2-[(E)-2-(3- methoxyphenyl)ethenyl]-5,6,9,10- tetrahydropyrazino[1′,2′:1,5]pyra- zolo[3,4-f]isoquinolin-7(8H)-one trifluoroacetate | 0.328 | 11.8 |
| 6 | 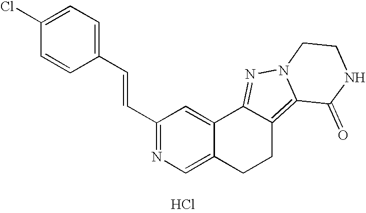 | 2-[(E)-2-(4-chlorophenyl)ethenyl]- 5,6,9,10- tetrahydropyrazino[1′,2′:1,5]pyra- zolo[3,4-f]isoquinolin-7(8H)-one hydrochloride | 0.536 | >20.0 |
| 7 | 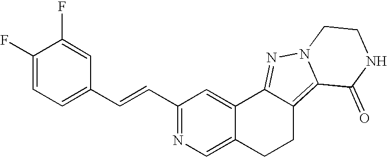 | 2-[(E)-2-(3,4- difluorophenyl)ethenyl]-5,6,9,10- tetrahydropyrazino[1′,2′:1,5]pyra- zolo[3,4-f]isoquinolin-7(8H)-one | 0.623 | >20.0 |
| 8 | 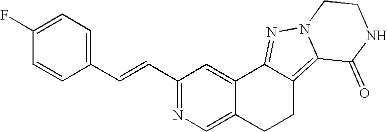 | 2-[(E)-2-(4-fluorophenyl)ethenyl]- 5,6,9,10- tetrahydropyrazino[1′,2′:1,5]pyra- zolo[3,4-f]isoquinolin-7(8H)-one | 0.749 | >20.0 |
| 9 | 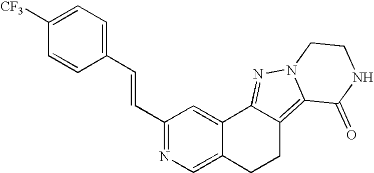 | 2-{(E)-2-[4- (trifluoromethyl)phenyl]ethenyl}- 5,6,9,10- tetrahydropyrazino[1′,2′:1,5]pyra- zolo[3,4-f]isoquinolin-7(8H)-one | 2.26 | >20.0 |
| 10 | 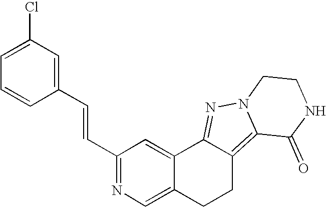 | 2-[(E)-2-(3-chlorophenyl)ethenyl]- 5,6,9,10- tetrahydropyrazino[1′,2′:1,5-pyra- zolo[3,4-f]isoquinolin-7(8H)-one | 2.92 | >20.0 |
| 11 | 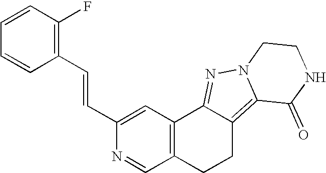 | 2-[(E)-2-(2-fluorophenyl)ethenyl]- 5,6,9,10- tetrahydropyrazino[1′,2′:1,5]pyra- zolo[3,4-f]isoquinolin-7(8H)-one | 4.46 | >200 |
| 12 | 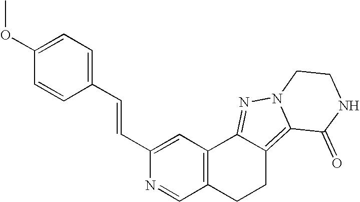 | 2-[(E)-2-(4-methoxyphenyl)vinyl]- 5,6,9,10- tetrahydropyrazino[1′,2′:1,5]pyra- zolo[3,4-f]isoquinolin-7(8H)-one | 4.76 | >200 |
| 13 | 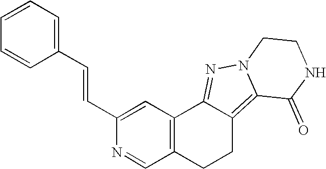 | 2-[(E)-2-phenylethenyl]-5,6,9,10- tetrahydropyrazino[1′,2′:1,5]pyra- zolo[3,4-f]isoquinolin-7(8H)-one | | |
| 14 | 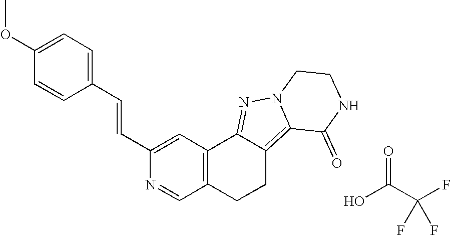 | 2-[(E)-2-(4-methoxyphenyl)vinyl]- 5,6,9,10- tetrahydropyrazino[1′,2′:1,5]pyra- zolo[3,4-f]isoquinolin-7(8H)-one trifluoroacetate | | |
| 15 | 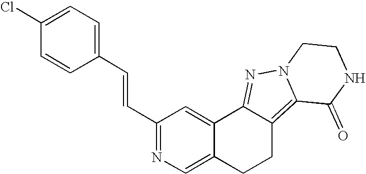 | 2-[(E)-2-(4-chlorophenyl)ethenyl]- 5,6,9,10- tetrahydropyrazino[1′,2′:1,5]pyra- zolo[3,4-f]isoquinolin-7(8H)-one | | |
| 16 | 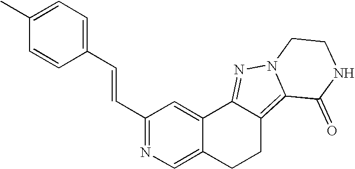 | 2-[(E)-2-(4-methylphenyl)ethenyl]- 5,6,9,10- tetrahydropyrazino[1′,2′:1,5]pyra- zolo[3,4-f]isoquinolin-7(8H)-one | | |
| 17 | 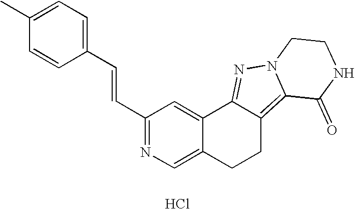 | 2-[(E)-2-(4-methylphenyl)ethenyl]- 5,6,9,10- tetrahydropyrazinop[1′,2′:1,5]pyra- zolo[3,4-f]isoquinolin-7(8H)-one hydrochloride | | |
| 18 | 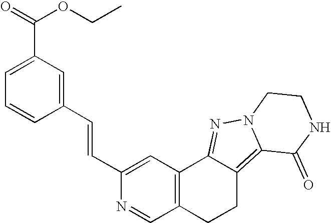 | ethyl 3-[(E)-2-(7-oxo-5,6,7,8,9,10- hexahydropyrazino[1′,2′:1,5]pyra- zolo[3,4-f]isoquinolin-2- yl)ethyl]benzoate | | |
| 19 | 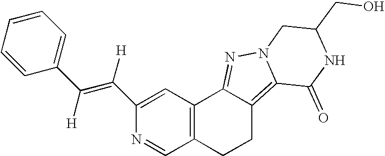 | 9-(hydroxymethyl)-2-[(E)-2- phenylvinyl]-5,6,9,10- tetrahydropyrazino[1′,2′:1,5]pyra- zolo[3,4-f]isoquinolin-7(8H)-one | | |
| 20 | 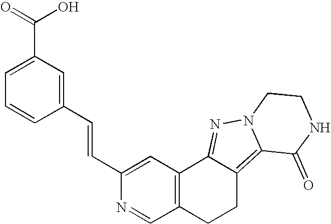 | 3-[(E)-2-(7-oxo-5,6,7,8,9,10- hexahydropyrazino[1′,2′:1,5]pyra- zolo[3,4-f]isoquinolin-2- yl)vinyl]benzoic acid | | |
| 21 | 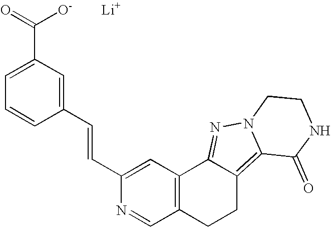 | lithium 3-[(E)-2-(7-oxo- 5,6,7,8,9,10- hexahydropyrazino[1′,2′:1,5]pyra- zolo[3,4-f]isoquinolin-2- yl)vinyl]benzoate | | |
| 22 | 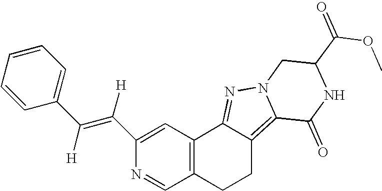 | methyl 7-oxo-2-[(E)-2-phenylvinyl]5,6,7,8,9,10- hexahydropyrazino[1′,2′:1,5]pyra- zolo[3,4-f]isoquinolin-9- carboxylate | | |
| 23 | 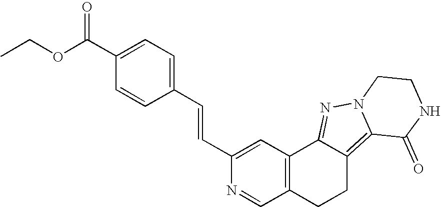 | ethyl 4-[(E)-2-(7-oxo-5,6,7,8,9,10- hexahydropyrazino[1′,2′:1,5]pyra- zolo[3,4-f]isoquinolin-2- yl)vinyl]benzoate | | |
| 24 | 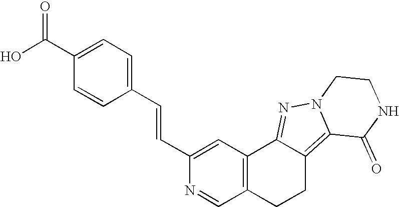 | 4-[(E)-2-(7-oxo-5,6,7,8,9,10- hexahydropyrazino[1′,2′:1,5]pyra- zolo[3,4-f]isoquinolin-2- yl)vinyl]benzoic acid | | |
| 25 | 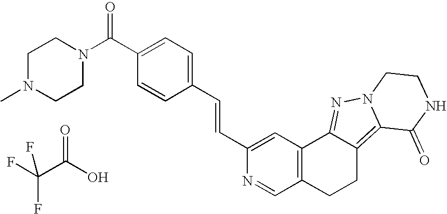 | 2-((E)-2-{4-[(4-methylpiperazin-1- yl)carbonyl]phenyl}vinyl)-5,6,9,10- tetrahydropyrazino[1′,2′:1,5]pyra- zolo[3,4-f]isoquinolin-7(8H)-one trifluoroacetate | | |
| 26 | 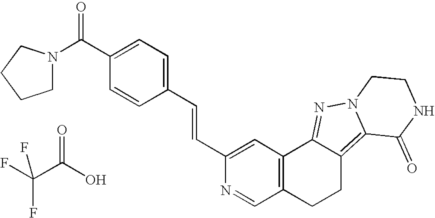 | 2-{(E)-2-[4-(pyrrolidin-1- ylcarbonyl)phenyl]vinyl}-5,6,9,10- tetrahydropyrazino[1′,2′:1,5]pyra- zolo[3,4-f]isoquinolin-7(8H)-one trifluoroacetate | | |
| 27 | 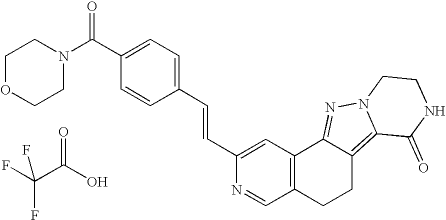 | 2-{(E)-2-[4-(morpholin-4- ylcarbonyl)phenyl]vinyl}-5,6,9,10- tetrahydropyrazino[1′,2′:1,5]pyra- zolo[3,4-f]isoquinolin-7(8H)-one trifluoroacetate | | |
| 28 | 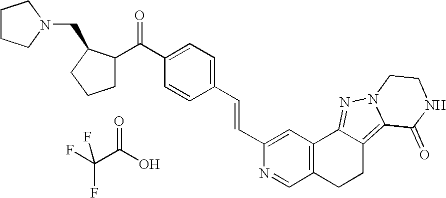 | 2-[(E)-2-(4-{[(2S)-2-(pyrrolidin-1- ylmethyl)pyrrolidin-1- yl]carbonyl}phenyl)vinyl]-5,6,9,10- tetrahydropyrazino[1′,2′:1,5]pyra- zolo[3,4-f]isoquinolin-7(8H)-one trifluoroacetate | | |
| 29 | 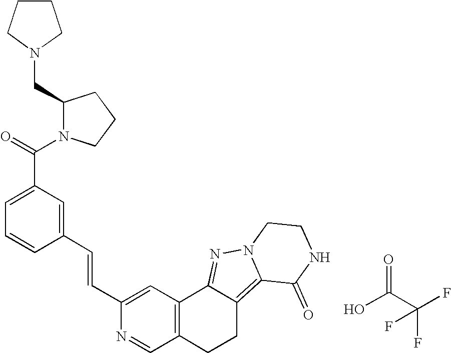 | 2-[(E)-2-(3-{[(2R)-2-(pyrrolidin-1- ylmethyl)pyrrolidin-1- yl]carbonyl}phenyl)vinyl]-5,6,9,10- tetrahydropyrazino[1′,2′:1,5]pyra- zolo[3,4-f]isoquinolin-7(8H)-one trifluoroacetate | | |
| 30 | 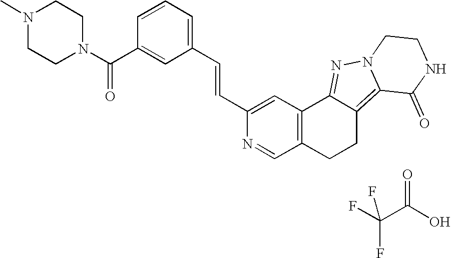 | 2-((E)-2-{3-[(4-methylpiperazin-1- yl)carbonyl]phenyl}vinyl)-5,6,9,10- tetrahydropyrazino[1′,2′:1,5]pyra- zo0lo[3,4-f]isoquinolin-7(8H)-one trifluoroacetate | | |
| 31 | 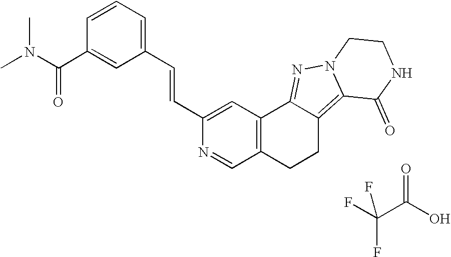 | N,N-dimethyl-3-[(E)-2-(7-oxo- 5,6,7,8,9,10- hexahydropyrazino[1′,2′:1,5]pyra- zolo[3,4-f]isoquinolin-2- yl)vinyl]benzamide trifluoroacetate | | |
| 32 | 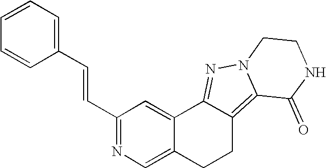 | 2-[(E)-2-phenylvinyl]-5,6,8,9,10,11- hexahydro-7H- [1,4]diazepino[1′,2′:1,5]pyrazolo[3,4- g]isoquinolin-7-one | | |
| 33 | 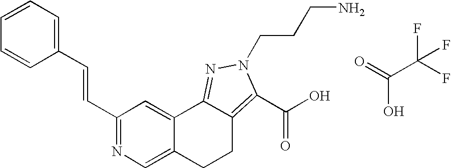 | 2-(3-aminopropyl)-8-[(E)-2- phenylvinyl]-4,5-dihydro-2H- pyrazolo[3,4-f]isoquinoline-3- carboxylic acid trifluoroacetate | 0.0329 | 0.528 |
| 34 | 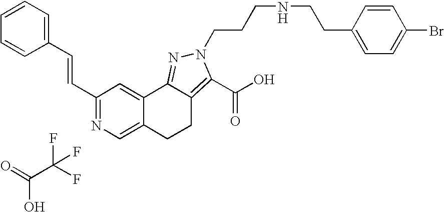 | 2-(3-{[2-(4- bromophenyl)ethyl]amino}propyl)- 8-[(E)-2-phenylvinyl]-4,5-dihydro- 2H-pyrazolo[3,4-f]isoquinoline-3- carboxylic acid trifluoroacetate | 0.28 | 2.02 |
| 35 | 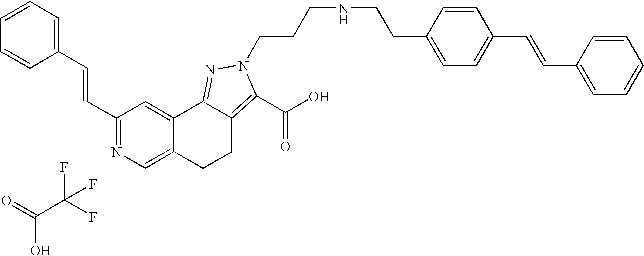 | 8-[(E)-2-phenylvinyl]-2-{3-[(2-{4- [(E)-2- phenylvinyl]phenyl}ethyl)amino]pro- pyl}-4,5-dihydro-2H-pyrazolo[3,4- f]isoquinoline-3-carboxylic acid trifluoroacetate | 0.528 | >20.0 |
| 36 | 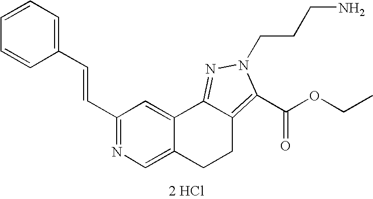 | ethyl 2-(3-aminopropyl)-8-[(E)-2- phenylvinyl]-4,5-dihydro-2H- pyrazolo[3,4-f]isoquinoline-3- carboxylate hydrochloride | | |
| 37 | 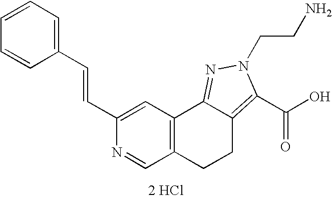 | 2-(2-aminoethyl)-8-[(E)-2- phenylethenyl]-4,5-dihydro-2H- pyrazolo[3,4-f]isoquinoline-3- carboxylic acid hydroxhloride | 0.0413 | 1.21 |
| 38 | 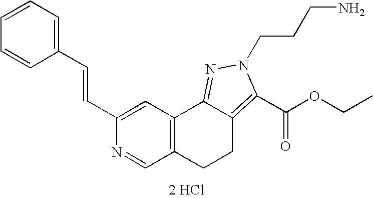 | ethyl 2-(2-aminoethyl)-8-[(E)-2- phenylethenyl]-4,5-dihydro-2H- pyrazolo[3,4-f]isoquinoline-3- carboxylate hydrochloride | 3.61 | >20.0 |

^{a}The cyclic pyrazole compound may be shown with a solvent, such as, for example, trifluoroacetate, with which it can for a salt. Both the salt and acid forms of the cyclic pyrazole compound are included in the present invention.
^{b}Compound names generated by ACD/Name software.

In some embodiments of the present invention, it is preferred that the cyclic pyrazole MK-2 inhibiting compounds have IC_{50 MK-2} values of less than 100, more preferred are those with IC_{50 MK-2} values of less than 50, even more preferred are those with IC_{50 MK-2} values of less than 20, yet more preferred are those with IC_{50 MK-2} values of less than 10, and even more preferred are those having IC_{50 MK-2} values of less than 1. Examples of cyclic pyrazole MK-2 inhibitors having such IC₅₀MK-2 values can be found in the tables above.

It should be understood that salts and prodrugs of the compounds that are described herein, as well as isomeric forms, tautomers, racemic mixtures of the compounds, and the like, which have the same or similar activity as the compounds that are described, are to be considered to be included within the description of the compound.

The MK-2 or MK-3 inhibiting activity of any of the compounds described herein can be determined by any one of several methods that are well known to those having skill in the art of enzyme activity testing. One method of measuring the MK-2 inhibiting activity is described in detail in the general methods section of the examples. A similar method, with appropriate modifications, Can be used to measure MK-3 inhibitory activity. In addition, the efficacy of one of the present MK-2 inhibiting compounds in therapeutic applications can be determined by testing for inhibition of TNFα production in cell culture and in animal model assays. In general, it is preferred that the MK-2 inhibiting compounds of the present invention be capable of inhibiting the production and/or the release of TNFα in cell cultures and in animal models.

In the present method, the MK-2 inhibiting compounds that are described herein can be used as inhibitors of MAPKAP kinase-2. When this inhibition is for a therapeutic purpose, one or more of the present compounds can be administered to a subject that is in need of MK-2 inhibition. As used herein, a “subject in need of MK-2 inhibition” is a subject who has, or who is at risk of contracting a TNFα mediated disease or disorder. TNFα mediated diseases and disorders are described in more detail below.

As described above, in an embodiment of the present method, a subject in need of prevention or treatment of a TNFα mediated disease or disorder is treated with one or more of the present MK-2 inhibiting compounds. In one embodiment, the subject is treated with an effective amount of the MK-2 inhibiting compound. The effective amount can be an amount that is sufficient for preventing or treating the TNFα mediated disease or disorder.

The MK-2 inhibiting compound that is used in the subject method can be any cyclic pyrazole MK-2 inhibiting compound that is described herein.

In the subject method, the MK-2 inhibiting compound can be used in any amount that is an effective amount. It is preferred, however, that the amount of the MK-2 inhibiting compound that is administered is within a range of about 0.1 mg/day per kilogram of the subject to about 1500 mg/day/kg. It is more preferred that the amount of the compound is within a range of about 1 mg/day/kg to about 500 mg/day/kg. An amount that is within a range of about 10 mg/day/kg to about 400 mg/day/kg, is even more preferred.

When the term “about” is used herein in relation to a dosage amount of the MK-2 inhibiting compound, it is to be understood to mean an amount that is within ±0.05 mg. By way of example, “about 0.1-10 mg/day” includes all dosages within 0.05 to 10.05 mg/day.

In another embodiment of the present invention, a pharmaceutical composition that contains one or more of the present cyclic pyrazole MK-2 inhibitors can be administered to a subject for the prevention or treatment of a TNFα mediated disease or disorder. The pharmaceutical composition includes a cyclic pyrazole MK-2 inhibitor of the present invention and a pharmaceutically acceptable carrier.

In another embodiment, a kit can be produced that is suitable for use in the prevention or treatment of a TNFα mediated disease or disorder. The kit comprises a dosage form comprising a cyclic pyrazole MK-2 inhibitor that is described herein in an amount which comprises a therapeutically effective amount.

As used herein, an “effective amount” means the dose or effective amount to be administered to a patient and the frequency of administration to the subject which is readily determined by one or ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used, the nature and severity of the illness to be treated, as well as the sex, age, weight, general health and individual responsiveness of the patient to be treated, and other relevant circumstances.

The phrase “therapeutically-effective” indicates the capability of an agent to prevent, or improve the severity of, the disorder, while avoiding adverse side effects typically associated with alternative therapies. The phrase “therapeutically-effective” is to be understood to be equivalent to the phrase “effective for the treatment, prevention, or inhibition”, and both are intended to qualify the amount of each agent for use in the combination therapy which will achieve the goal of improvement in the severity of pain and inflammation and the frequency of incidence over treatment of each agent by itself, while avoiding adverse side effects typically associated with alternative therapies.

Those skilled in the art will appreciate that dosages may also be determined with guidance from Goodman & Goldman's The Pharmacological Basis of Therapeutics, Ninth Edition (1996), Appendix 11, pp. 1707-1711.

The frequency of dose will depend upon the half-life of the active components of the composition. If the active molecules have a short half life (e.g. from about 2 to 10 hours) it may be necessary to give one or more doses per day. Alternatively, if the active molecules have a long half-life (e.g. from about 2 to about 15 days) it may only be necessary to give a dosage once per day, per week, or even once every 1 or 2 months. A preferred dosage rate is to administer the dosage amounts described above to a subject once per day.

For the purposes of calculating and expressing a dosage rate, all dosages that are expressed herein are calculated on an average amount-per-day basis irrespective of the dosage rate. For example, one 100 mg dosage of an MK-2 inhibitor taken once every two days would be expressed as a dosage rate of 50 mg/day. Similarly, the dosage rate of an ingredient where 50 mg is taken twice per day would be expressed as a dosage rate of 100 mg/day.

For purposes of calculation of dosage amounts, the weight of a normal adult human will be assumed to be 70 kg.

When the MK-2 inhibitor is supplied along with a pharmaceutically acceptable carrier, the pharmaceutical compositions that are described above can be formed. Pharmaceutically acceptable carriers include, but are not limited to, physiological saline, Ringer's, phosphate solution or buffer, buffered saline, and other carriers known in the art. Pharmaceutical compositions may also include stabilizers, anti-oxidants, Colorants, and diluents. Pharmaceutically acceptable carriers and additives are chosen such that side effects from the pharmaceutical compound are minimized and the performance of the compound is not canceled or inhibited to such an extent that treatment is ineffective.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, Calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, Choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in the combination of the invention are the isomeric forms and tautomers and the pharmaceutically-acceptable salts of the present MK-2 inhibitors. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, P-hydroxybutyric, galactaric and galacturonic acids.

Suitable pharmaceutically-acceptable base addition salts of compounds of the present invention include metallic ion salts and organic ion salts. More preferred metallic ion salts include, but are not limited to, appropriate alkali metal (group Ia) salts, alkaline earth metal (group IIa) salts and other physiological acceptable metal ions. Such salts can be made from the ions of aluminum, Calcium, lithium, magnesium, potassium, sodium and zinc. Preferred organic salts can be made from tertiary amines and quaternary ammonium salts, including in part, trifluoroacetate, trimethylamine, diethylamine, N,N′-dibenzylethylenediamine, Chloroprocaine, Choline, diethanolamine, ethylenediamine, meglumine (N-methylglucamine) and procaine. All of the above salts can be prepared by those skilled in the art by conventional means from the corresponding compound of the present invention.

The method of the present invention is useful for, but not limited to, the prevention and treatment of diseases and disorders that are mediated by TNFα. For example, the MK-2 inhibitors of the present invention could be useful to treat arthritis, including, but not limited to, rheumatoid arthritis, spondyloarthopathies, gouty arthritis, osteoarthritis, systemic lupus erythematosus and juvenile arthritis. Such MK-2 inhibiting compounds of the invention would be useful in the treatment of asthma, bronchitis, menstrual cramps, tendinitis, bursitis, connective tissue injuries or disorders, and skin related conditions such as psoriasis, eczema, burns and dermatitis.

The MK-2 inhibiting compounds that are useful in the method of the invention also would be useful to treat gastrointestinal conditions such as inflammatory bowel disease, gastric ulcer, gastric varices, Crohn's disease, gastritis, irritable bowel syndrome and ulcerative colitis and for the prevention or treatment of cancer, such as colorectal cancer. Such MK-2 inhibiting compounds would be useful in treating inflammation in diseases and conditions such as herpes simplex infections, HIV, pulmonary edema, kidney stones, minor injuries, wound healing, vaginitis, candidiasis, lumbar spondylanhrosis, lumbar spondylarthrosis, vascular diseases, migraine headaches, sinus headaches, tension headaches, dental pain, periarteritis nodosa, thyroiditis, aplastic anemia, Hodgkin's disease, sclerodoma, rheumatic fever, type I diabetes, myasthenia gravis, multiple sclerosis, sarcoidosis, nephrotic syndrome, Behcet's syndrome, polymyositis, gingivitis, hypersensitivity, swelling occurring after injury, myocardial ischemia, and the like.

The present MK-2 inhibitors would also be useful in the treatment of ophthalmic diseases, such as retinitis, retinopathies, conjunctivitis, uveitis, ocular photophobia, and of acute injury to the eye tissue. These compounds would also be useful in the treatment of pulmonary inflammation, such as that associated with viral infections and cystic fibrosis. The compounds would also be useful for the treatment of certain central nervous system disorders such as cortical dementias including Alzheimer's disease.

As used herein, the terms “TNFα mediated disease or disorder” are meant to include, without limitation, each of the symptoms or diseases that is mentioned above.

The terms “treating” or “to treat” mean to alleviate symptoms, eliminate the causation either on a temporary or permanent basis, or to prevent or slow the appearance of symptoms. The term “treatment” includes alleviation, elimination of causation of or prevention of pain and/or inflammation associated with, but not limited to, any of the diseases or disorders described herein. Besides being useful for human treatment, these combinations are also useful for treatment of mammals, including horses, dogs, Cats, rats, mice, sheep, pigs, etc.

The term “subject” for purposes of treatment includes any human or animal subject who is in need of the prevention of or treatment of any one of the TNFα mediated diseases or disorders. The subject is typically a mammal. “Mammal”, as that term is used herein, refers to any animal classified as a mammal, including-humans, domestic and farm animals, and zoo, sports, or pet animals, such as dogs, horses, Cats, Cattle, etc., Preferably, the mammal is a human.

For methods of prevention, the subject is any human or animal subject, and preferably is a subject that is in need of prevention and/or treatment of a TNFα mediated diseases or disorders. The subject may be a human subject who is at risk of obtaining a TNFα mediated disease or disorder, such as those described above. The subject may be at risk due to genetic predisposition, sedentary lifestyle, diet, exposure to disorder-causing agents, exposure to pathogenic agents and the like.

The subject pharmaceutical compositions may be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

In particular, the pharmaceutical compositions of the present invention can be administered orally, for example, as tablets, Coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, Coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, Calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, Calcium carbonate, Calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the MK-2 inhibitors in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols; for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel combination may be formulated with sweetening agents, for example glycerol, sorbitol or sucrose. Such formulations may also contain a demulcent, a preservative and flavoring and coloring agents.

The subject compositions can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-, or di-, glycerides. In addition, n-3 polyunsaturated fatty acids may find use in the preparation of injectables.

The subject compositions can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The novel compositions can also be administered topically, in the form of creams, ointments, jellies, collyriums, solutions or suspensions. Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Various delivery systems include capsules, tablets, and gelatin capsules, for example.

The following examples describe preferred embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples all percentages are given on a weight basis unless otherwise indicated.

### General Information for Preparation Methods:

Unless otherwise noted, reagents and solvents were used as received from commercial suppliers.

NMR Analysis:

Proton nuclear magnetic resonance spectra were obtained on a Varian Unity Innova 400, a Varian Unity Innova 300 a Varian Unity 300, a Bruker AMX 500 or a Bruker AV-300 spectrometer. Chemical shifts are given in ppm (δ) and coupling constants, J, are reported in Hertz. Tetramethylsilane was used as an internal standard for proton spectra and the solvent peak was used as the reference peak for carbon spectra. Mass spectra were obtained on a Perkin Elmer Sciex 100 atmospheric pressure ionization (APCI) mass spectrometer, a Finnigan LCQ Duo LCMS ion trap electrospray ionization (ES I) mass spectrometer, a PerSeptive Biosystems Mariner TOF HPLC-MS (ESI), or a Waters ZQ mass spectrometer (ESI).

Determination of MK-2 IC₅₀:

Recombinant MAPKAPK2 was phosphorylated at a concentration of 42-78 μM by incubation with 0.23 μM of active p38α in 50 mM HEPES, 0.1 mM EDTA, 10 mM magnesium acetate, and 0.25 mM ATP, pH 7.5 for one hour at 30° C.

The phosphorylation of HSP-peptide (KKKALSRQLSVAA) by MAPKAPK2 was measured using an anion exchange resin capture assay method. The reaction was carried out in 50 mM β-glycerolphosphate, 0.04% BSA, 10 mM magnesium acetate, 2% DMSO and 0.8 mM dithiotheritol, pH 7.5 in the presence of the HSP-peptide with 0.2 μCi [γ³³P]ATP and 0.03 mM ATP. The reaction was initiated by the addition of 15 nM MAPKAPK2 and was allowed to incubate at 30° C. for 30 min. The reaction was terminated and [γ³³P]ATP was removed from solution by the addition of 150 μl of AG 1×8 ion exchange resin in 900 mM sodium formate pH 3.0. A 50 μl aliquot of head volume was removed from the quenched reaction mixture and added to a 96-well plate, 150 μl of Microscint-40 (Packard) was added and the amount of phosphorylated-peptide was determined. Allow the Microscint to sit in the plates for 60 minutes prior to counting.

Compounds are evaluated as potential inhibitors of the MK-2 kinase by measuring their effects on MK-2 phosphorylation of the peptide substrate. Compounds may be screened initially at two concentrations prior to determination of IC₅₀ values. Screening results are expressed as percent inhibition at the concentrations of compound tested. For IC₅₀ value determinations, Compounds are tested at six concentrations in ten-fold serial dilutions with each concentration tested in triplicate. Results are expressed as IC₅₀ values in micromolar. The assay is performed at a final concentration of 2% DMSO.

U937 Cell TNFα Release Assay

The human monocyte-like cell line, U937 (ATCC #CRL-1593.2), is cultured in RPMI1640 media with 10% heat-inactivated fetal calf serum (GIBCO), glutamine and pen/strep at 37° C. and 5% CO₂. Differentiation of U937 to monocytic/macrophage-like cells is induced by the addition of phorbol12-myristate 13-acetate (Sigma) at final concentration of 20 ng/ml to a culture of U937 cells at ˜0.5 million cells/ml and incubated for 24 hrs. The cells are centrifuged, washed with PBS and resuspended in fresh media without PMA and incubated for 24 hrs. Cells adherent to the culture flask are harvested by scraping, Centrifugation, and resuspended in fresh media to 2 million cells/ml, and 0.2 ml is aliquoted to each of 96 wells in flat-bottom plate. Cells are then incubated for an additional 24 hrs to allow for recovery. The media is removed from the cells, and 0.1 ml of fresh media is added per well. 0.05 ml of serially diluted compound or control vehicle (Media with DMSO) is added to the cells. The final DMSO concentration does not exceed 1%. After 1 hr incubation, 0.05 ml of 400 ng/ml LPS (E Coli serotype 0111:B4, Sigma) in media is added for final concentration of 100 ng/ml. Cells are incubated at 37° C. for 4 hrs. After 4 hrs incubation, supernatants are harvest and assayed by ELISA for the presence of TNFα.

U937 Cell TNFα ELISA

ELISA plates (NUNC-Immuno™ Plate Maxisorb™ Surface) were coated with purified mouse monoclonal IgG1 anti-human TNFα antibody (R&D Systems #MAB610; 1.25 ug/ml in sodium bicarbonate pH 8.0, 0.1 ml/well) and incubated at 4° C. Coating solution was aspirated the following day and wells were blocked with 1 mg/ml gelatin in PBS (plus 1× thimerasol) for 2 days at 4° C. Prior to using, wells were washed 3× with wash buffer (PBS with 0.05% Tween). Cultured media samples were diluted in EIA buffer (5 mg/ml bovine γ-globulin, 1 mg/ml gelatin, 1 ml/l Tween-20, 1 mg/ml thimerasol in PBS), added to wells (0.1 ml/well) in triplicate and allowed to incubate for 1.5 hr at 37° C. in a humidified chamber. Plates were again washed and 0.1 ml/well of a mixture of rabbit anti-human TNFα polyclonal antibodies in EIA buffer (1:400 dilution of Sigma #T8300, and 1:400 dilution of Calbiochem #654250) was added for 1 hr at 37° C. Plates were washed as before and peroxidase-conjugated goat anti-rabbit IgG (H+L) antibody (Jackson ImmunoResearch #111-035-144, 1 ug/ml in EIA buffer, 0.1 ml/well) was added for 45 min. After final washing, plates were developed with peroxidase-ABTS solution (Kirkegaard/Perry #50-66-01, 0.1 ml/well). Enzymatic conversion of ABTS to colored product was measured after 5-30 minutes using a SpectroMax 340 spectrophotometer (Molecular Devices) at 405 nm. TNF levels were quantitated from a recombinant human TNFα (R&D Systems #210-TA-010) standard curve using a quadratic parameter fit generated by SoftMaxPRO software. ELISA sensitivity was approximately 30 pg TNF/ml. IC₅₀ values for compounds were generated using BioAssay Solver.

Lipopolysaccharide (LPS)-Induced TNFα Production.

Adult male 225-250 gram Lewis rats (Harlan Sprague-Dawley) were used. Rats were fasted 18 hr prior to oral dosing, and allowed free access to water throughout the experiment. Each treatment group consisted of 5 animals.

Compounds were prepared as a suspension in a vehicle consisting of 0.5% methylcellulose, 0.025% Tween-20 in PBS. Compounds or vehicle were orally administered in a volume of 1 ml using an 18 gauge gavage needle. LPS (E. coli serotype 0111:B4, Lot #39H4103, Cat. # L-2630, Sigma) was administered 1-4 hr later by injection into the penile vein at a dose of 1 mg/kg in 0.5 ml sterile saline. Blood was collected in serum separator tubes via cardiac puncture 1.5 hr after LPS injection, a time point corresponding to maximal TNFα production. After clotting, serum was withdrawn and stored at −20° C. until assay by ELISA (described below).

Rat LPS TNFα ELISA

ELISA plates (NUNC-Immuno™ Plate Maxisorb™ Surface) were coated with 0.1 ml per well of an Protein G purified fraction of a 2.5 ug/ml of hamster anti-mouse/rat TNFα monoclonal antibody TN19.12 (2.5 ug/ml in PBS, 0.1 ml/well). The hybridoma cell line was kindly provided by Dr. Robert Schreiber, Washington University. Wells were blocked the following day with 1 mg/ml gelatin in PBS. Serum samples were diluted in a buffer consisting of 5 mg/ml bovine γ-globulin, 1 mg/ml gelatin, 1 ml/l Tween-20, 1 mg/ml thimerasol in PBS, and 0.1 ml of diluted serum was added wells in duplicate and allowed to incubate for 2 hr at 37° C. Plates were washed with PBS-Tween, and 0.1 ml per well of a 1:300 dilution of rabbit anti-mouse/rat TNFα antibody (BioSource International, Cat. #AMC3012) was added for 1.5 hr at 37° C. Plates were washed, and a 1:1000 fold dilution of peroxidase-conjugated donkey anti-rabbit IgG antibody (Jackson ImmunoResearch, Cat. #711-035-152) was added for 45 min. After washing, plates were developed with 0.1 ml of ABTS-peroxide solution (Kirkegaard/Perry, Cat. #50-66-01). Enzymatic conversion of ABTS to colored product was measured after 30 minutes using a SpectroMax 340 spectrophotometer (Molecular Devices corp.) at 405 nm. TNF levels in serum were quantitated from a recombinant rat TNFα (BioSource International, Cat. #PRC3014.) standard curve using a quadratic parameter fit generated by SoftMaxPRO software. ELISA sensitivity was approximately 30 pg TNF/ml. Results are expressed in percent inhibition of the production of TNFα as compared to blood collected from control animals dosed only with vehicle.

### Synthesis of MK-2 Inhibiting Compounds of the Present Invention

### EXAMPLE 1

This example illustrates the production of 6-hydroxy-2,4-dihydroindeno[1,2-c]pyrazole-3-carboxylic acid.

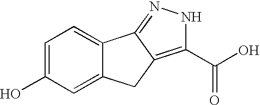

Step 1: The Preparation of Methyl (5-methoxy-1-oxo-2,3-dihydro-1H-inden-2-yl)(oxo)acetate:

5-methoxyindan-1-one (30 mmol, 4.86 g), ether (150 mL), sodium methoxide (25%/methanol, 33 mmol, 7.13 g), and dimethyl oxalate (33 mmol, 3.90 g) were stirred at room temperature for 16 h. Hydrochloric acid (1 N) was added until the mixture was acidic to pH paper. The mixture was extracted with hot ethyl acetate (3×100 mL). The combined organic extracts were washed with brine 2×, dried over magnesium sulfate, and concentrated. This material was used in the next step without further purification or characterization.

Step 2: The Preparation of Methyl 6-methoxy-2,4-dihydroindeno[1,2-c]pyrazole-3-carboxylate.

To methyl (5-methoxy-1-oxo-2,3-dihydro-1H-inden-2-yl)(oxo)acetate from step 1 was added hydrazine hydrochloride, the material from the previous step, (33 mmol, 2.26 g) and methanol (100 mL). This mixture was stirred at reflux for 1 h. After cooling to room temperature, water was added until the mixture became cloudy. After standing for 2 h a yellow solid was filtered (3.75 g). ¹H NMR (DMSO-d₆/300 MHz) 13.6 (brs, 1H), 7.5 (d, 1H), 7.14 (s, 1H), 6.9 (d, 1H), 3.84 (s, 2H), 3.76 (s, 3H) 3.70 (s, 3H). ESHRMS m/z 245.090 (M+H, C₁₃H₁₂N₂O₃ requires 307.130).

Step 3: The Preparation of Methyl 6-hydroxy-2,4-dihydroindeno[1,2-c]pyrazole-3-carboxylate.

Methyl 6-methoxy-2,4-dihydroindeno[1,2-c]pyrazole-3-carboxylate (1 mmol, 244 Mg) and dichloromethane (10 mL) were stirred at −78° under nitrogen. Boron tribromide (1.0 N/dichloromethane, 5 mmol, 5 mL) was added drop wise and the mixture was allowed to slowly warm to room temperature. The mixture was re-cooled to −78° and boron tribromide (1.0 N/dichloromethane, 5 mmol, 5 mL) was added. This sequence was repeated a third time and the mixture was stirred at room temperature 16 h. Methanol (25 mL) was added drop wise with a large exotherm. The mixture was then concentrated to 10 mL, diluted with methanol and re-concentrated three times. Ethyl acetate (100 mL) was added followed by the slow addition of saturated aqueous sodium bicarbonate to neutral pH. Citric acid (1 N) was added to adjust the pH to 4 and the layers were separated. The aqueous layer was then extracted with ethyl acetate (3×30 mL). The combined organics were washed with brine 2×, dried over magnesium sulfate, and concentrated. The product was recrystallized from ethyl acetate/hexanes to yield a tan solid (140 Mg). ¹H NMR (DMSO-d₆/400 MHz) 13.5 (brs, 1H), 7.41 (m, 1H), 6.94 (s, 1H), 6.72 (dd, 1H), 3.82 (s, 3H), 3.64 (s, 2H). ESHRMS m/z 231.076 (M+H, C₁₂H₁₀N₂O₃ requires 231.076).

Step 4: The Preparation of 6-hydroxy-2,4-dihydroindeno[1,2-c]pyrazole-3-carboxylic Acid.

Methyl 6-hydroxy-2,4-dihydroindeno[1,2-c]pyrazole-3-carboxylate (110 Mg), methanol (5 mL), water (1 mL), and sodium hydroxide (2.5 N, 1 mL) were stirred at room temperature overnight. The mixture was then concentrated to 4 mL and citric acid (1 N) was added to adjust the pH to 4. A white solid was filtered (97 Mg). ¹H NMR (DMSO-d₆/400 MHz) 13.2 (brs, 1H), 9.48 (s, 1H), 7.39 (m, 1H), 6.92 (s, 1H), 6.72 (dd, 1H), 3.30 (s, 2H). ESHRMS m/z 217.064 (M+H, C₁₁H₈N₂O₃ requires m.w. 217.061).

### EXAMPLE 2

This example illustrates the production of 2-(3-aminopropyl)-6-hydroxy-2,4-dihydroindeno[1,2-c]pyrazole-3-carboxylic acid hydrobromide.

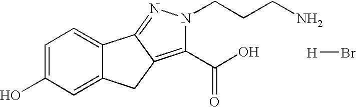

Step 1: The Preparation of tert-butyl 2-{3-[(tert-butoxycarbonyl)amino]propyl}-6-methoxy-2,4-dihydroindeno[1,2-c]pyrazole-3-carboxylate.

Methyl 6-methoxy-2,4-dihydroindeno[1,2-c]pyrazole-3-carboxylate (10 mmol, 2.44 g) and N,N′-dimethylformamide (100 mL) were stirred at 0° under nitrogen. Lithium t-butoxide (1.0 N/tetrahydrofuran, 20 mmol, 20 mL) was slowly added. After 30 min., tert-butyl 3-bromopropylcarbamate (15 mmol, 3.57 g) was added and the mixture was stirred at room temperature for 16 h. Saturated ammonium chloride solution was added and the mixture was extracted with ethyl acetate (3×75 mL). The combined organics were washed with brine 2×, dried over magnesium sulfate, and concentrated. Purification was by flash column chromatography. The appropriate fractions were concentrated to a viscous yellow oil (0.86 g). ¹H NMR (acetone-d₆/400 MHz) 7.55 (d, 1H), 7.14 (d, 1H), 6.92 (dd, 1H), 5.96 (brs, 1H), 4.61 (t, 2H), 3.83 (s, 3H), 3.72 (s, 2H), 3.11 (dd, 2H), 2.03 (m, 2H), 1.62 (s, 9H), 1.38 (s, 9H). ESHRMS m/z 444.250 (M+H, C₂₄H₃₃N₃O₅ requires m.w. 444.249).

Step 2: The Preparation of 2-(3-aminopropyl)-6-hydroxy-2,4-dihydroindeno[1,2-c]pyrazole-3-carboxylic Acid Hydrobromide.

Tert-butyl 2-{3-[(tert-butoxycarbonyl)amino]propyl}-6-methoxy-2,4-dihydroindeno[1,2-c]pyrazole-3-carboxylate (1 mmol, 444 Mg) and dichloromethane (15 mL) were stirred at −78° under nitrogen. Boron tribromide (1.0 N/dichloromethane, 20 mmol, 20 mL) was added drop wise and the mixture was allowed to stir at room temperature for 16 h. Methanol (10 mL) was added drop wise. The mixture was concentrated to 5 mL, diluted with methanol, and re-concentrated 2×. Anhydrous ethyl ether was added to precipitate a white solid, which was rinsed with ether/methanol (340 Mg). ¹H NMR (DMSO-d₆/400 MHz) 7.66 (brs, 2H), 7.39 (d, 1H), 6.94 (d, 1H), 6.73 (dd, 1H), 4.56 (t, 2H), 3.65 (s, 2H), 2.75 (m, 2H), 2.05 (q, 2H). ESHRMS m/z 274.119 (M+H, C₁₄H₁₅N₃O₃ requires 274.119).

### EXAMPLE 3

This example illustrates the production of 2-(3-aminopropyl)-7-hydroxy-2,4-dihydroindeno[1,2-c]pyrazole-3-carboxylic acid hydrobromide.

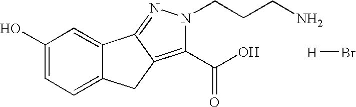

Step 1: The Preparation of Methyl (2Z)-hydroxy(6-methoxy-1-oxo-1,3-dihydro-2H-inden-2-ylidene)ethanoate.

6-methoxyindanone (60 mmol, 9.73 g), ether (200 mL), sodium methoxide (25%/methanol, 66 mmol, 14.26 g), and dimethyl oxalate (66 mmol, 7.79 g) were stirred at room temperature overnight. Hydrochloric acid (1 N, 75 ml) was added and the mixture was extracted with hot ethyl acetate (3×100 mL). The heated organics were washed with brine 2×, dried over magnesium sulfate, concentrated, and recrystallized from ether/hexanes. A yellow solid was collected by filtration (11.6 g). ¹H NMR (acetone-d₆/400 MHz) 7.55 (dd, 1H), 7.29 (dd, 1H), 7.25 (d, 1H), 3.90 (s, 3H), 3.88 (s, 3H), 3.86 (s, 2H) ESHRMS m/z 249.074 (M+H, C₁₃H₁₂O₅ requires m.w. 249.076).

Step 2: The Preparation of Methyl 7-methoxy-2,4-dihydroindeno[1,2-c]pyrazole-3-carboxylate.

Methyl (2Z)-hydroxy(6-methoxy-1-oxo-1,3-dihydro-2H-inden-2-ylidene)ethanoate (46 mmol, 11.5 g), methanol (150 mL), and hydrazine mono hydrochloride (51 mmol, 3.49 g) were heated to reflux for one hour. After cooling to room temperature, saturated aqueous sodium bicarbonate solution was added to neutral pH followed by water (100 mL). A dark yellow solid was collected by filtration (10.9 g). ¹H NMR (acetone-d₆/400 MHz) 12.85 (brs, 1H), 7.43 (d, 1H), 7.25 (d, 1H), 6.87 (dd, 1H), 3.90 (s, 3H), 3.85 (s, 3H), 3.69 (s, 2H). ESHRMS m/z 245.091 (M+H, C₁₃H₁₂N₂O₃ requires m.w. 245.092).

Step 3: The Preparation of tert-butyl 2-{3-[(tert-butoxycarbonyl)amino]propyl}-7-methoxy-2,4-dihydroindeno[1,2-c]pyrazole-3-carboxylate.

Methyl 7-methoxy-2,4-dihydroindeno[1,2-c]pyrazole-3-carboxylate (30 mmol, 7.33 g) and N,N′-dimethylformamide (100 mL) were stirred at 0° under nitrogen. Lithium t-butoxide (1.0 N/tetrahydrofuran, 66 mmol, 66 mL) was slowly added. After 30 min., tert-butyl 3-bromopropylcarbamate (15 mmol, 3.57 g) was added and the mixture was stirred at room temperature for 16 h. Saturated ammonium chloride solution was added and the mixture was extracted with ethyl acetate (3×50 mL). The combined organics were washed with brine 2×, dried over magnesium sulfate, and concentrated. Purification was by flash column chromatography eluting with ethyl acetate/hexanes. The appropriate fractions were concentrated to a viscous yellow oil (4.2 g). ¹H NMR (acetone-d₆/400 MHz) 7.41 (d, 1H), 7.22 (d, 1H), 6.85 (dd, 1H), 5.96 (brs, 1H), 4.64 (t, 2H), 3.85 (s, 3H), 3.67 (s, 2H), 3.12 (dd, 2H), 2.05 (m, 2H), 1.62 (s, 9H), 1.38 (s, 9H). ESHRMS m/z 444.248 (M+H, C₂₄H₃₃N₃O₅ requires m.w. 444.249).

Step 4: The Preparation of 2-(3-aminopropyl)-7-hydroxy-2,4-dihydroindeno[1,2-c]pyrazole-3-carboxylic Acid Hydrobromide.

Tert-butyl 2-{3-[(tert-butoxycarbonyl)amino]propyl}-7-methoxy-2,4-dihydroindeno[1,2-c]pyrazole-3-carboxylate (1 mmol, 444 Mg) and dichloromethane (10 mL) were stirred at −78° under nitrogen. Boron tribromide (1.0 N/dichloromethane, 15 mmol, 15 mL) was added drop wise and the mixture was allowed to stir at room temperature for 6 h when it was cooled to −40° and methanol (20 mL) was added drop wise. The mixture was concentrated to 5 mL, diluted with methanol, and re-concentrated 2×. Anhydrous ethyl ether was added to precipitate a white solid, which was rinsed with ether and air-dried (350 Mg). ¹H NMR (DMSO-d₆/400 MHz) 7.68 (brs, 2H), 7.29 (d, 1H), 6.99 (d, 1H), 6.68 (dd, 1H), 4.59 (t, 2H), 3.60 (s, 2H), 2.76 (m, 2H), 2.07 (q, 2H). ESHRMS m/z 274.118 (M+H, C₁₄H₁₅N₃O₃ requires m.w. 274.119).

### EXAMPLE 4

This example illustrates the production of 2-(3-aminopropyl)-7-hydroxy-2,5-dihydroisothiochromeno[4,3-c]pyrazole-3-carboxylic acid hydrochloride.

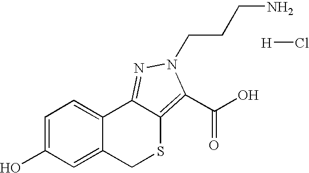

Step 1: The Preparation of [(3-methoxybenzyl)thio]acetic Acid.

3-methoxybenzyl chloride (100 mmol, 15.66 g), methanol (40 mL), and thiourea (100 mmol, 7.61 g) were stirred at reflux for 16 h. After cooling to room temperature, water (300 mL) and saturated aqueous sodium bicarbonate solution was added until neutral to pH paper. This mixture was allowed to stand at room temperature for 16 h. and a white solid was collected by filtration (15.0 g). To the resultant white solid was added sodium chloroacetate (115 mmol, 13.4 g), sodium hydroxide (2.5 N, 306 mmol, 122 mL), and methanol (150 mL). After heating to reflux for 16 h. and cooling to room temperature, concentrated hydrochloric acid was carefully added until the mixture was acidic to pH paper. The mixture was then extracted with ether (3×100 mL). The combined ethereal extracts were washed with brine 2×, dried over magnesium sulfate, and concentrated to a light brown oil (17.3 g). ¹H NMR (acetone-d₆/400 MHz) 7.22 (t, 1H), 6.92 (m, 2H), 6.81 (m, 1H), 3.83 (s, 2H), 3.78 (s, 3H), 3.13 (s, 2H).

Step 2: The Preparation of 7-methoxy-1H-isothiochromen-4(3H)-one.

[(3-methoxybenzyl)thio]acetic acid (81 mmol, 17.3 g) and trifluoroacetic acid (20 mL) were cooled to 0° with stirring. Trifluoroacetic anhydride (40 mL) was carefully added. After stirring for 3 h. at 00, water (100 mL) was added. The mixture was extracted with ethyl acetate (3×65 mL). The combined organics were washed with brine, saturated aqueous sodium bicarbonate, and brine 2×, maintaining an acidic pH throughout the process. The mixture was then dried over magnesium sulfate and concentrated under high vacuum to dryness. Purification was via re-crystallization from ether/ethyl acetate/hexanes to yield a black solid (4.23 g).

Step 3: The Preparation of Methyl 7-methoxy-2,5-dihydroisothiochromeno[4,3-c]pyrazole-3-carboxylate.

7-methoxy-1H-isothiochromen-4(3H)-one (the crude material from step 2, 22 mmol, 4.23), ether (100 mL), sodium methoxide (25%/methanol, 24 mmol, 5.18 g), and dimethyl oxalate (24 mmol, 2.83 g) were stirred at room temperature for 16 h. Hydrochloric acid (1 N) was added to pH 1 and the ethereal layers were separated. The aqueous layer was extracted with ethyl acetate (3×50 mL). The combined organic layers were washed with brine 2×, concentrated and recrystalized from ethyl acetate/hexanes to yield a black solid (3.46 g). To this crude material was added hydrazine mono hydrochloride (13.2 mmol, 0.9 g) and methanol (75 mL). The mixture was heated to reflux with stirring for 16 h. After cooling to room temperature, saturated aqueous sodium bicarbonate solution was added to neutral pH followed by water until the mixture became cloudy. After standing, a brown solid was obtained by filtration (2.92 g). ¹H NMR (acetone-d₆/400 MHz) 12.95 (brs, 1H), 7.75 (s, 1H), 6.95 (m, 2H), 4.00 (s, 2H), 3.87 (s, 3H), 3.83 (s, 3H). ESHRMS m/z 277.064 (M+H, C₁₃H₁₂N₂O₃S requires m.w. 277.064).

Step 4: The Preparation of Methyl 2-{3-[(tert-butoxycarbonyl)amino]propyl}-7-methoxy-2,5-dihydroisothiochromeno[4,3-c]pyrazole-3-carboxylate.

Methyl 7-methoxy-2,5-dihydroisothiochromeno[4,3-c]pyrazole-3-carboxylate (8 mmol, 2.21 g) and N,N′-dimethylformamide (50 mL) were stirred at 0° under nitrogen. Lithium t-butoxide (1.0 N/tetrahydrofuran, 16 mmol, 16 mL) was slowly added. After 30 min., tert-butyl 3-bromopropylcarbamate (16 mmol, 3.81 g) was added and the mixture was stirred at room temperature for 16 h. Saturated ammonium chloride solution was added and the mixture was extracted with ethyl acetate (3×50 mL). The combined organics were washed with brine 2×, dried over magnesium sulfate, and concentrated. Purification was by flash column chromatography eluting with ethyl acetate/hexanes. The appropriate fractions were concentrated to a clear oil (190 Mg). This material was used in the next step without further purification or characterization.

Step 5: The Preparation of 2-(3-aminopropyl)-7-hydroxy-2,5-dihydroisothiochromeno[4,3-c]pyrazole-3-carboxylic Acid Hydrochloride.

Methyl 2-{3-[(tert-butoxycarbonyl)amino]propyl}-7-methoxy-2,5-dihydroisothiochromeno[4,3-c]pyrazole-3-carboxylate, the crude material from the previous step, (0.4 mmol, 173 Mg) and dichloromethane (5 mL) were stirred at −78° under nitrogen. Boron tribromide (1.0 M/dichloromethane, 4 mmol, 4 mL) was slowly added. After stirring at room temperature for 3 h., the mixture was cooled to −10° and methanol was added drop wise to a total volume of 30 mL. This dilute mixture was concentrated to 5 mL, diluted with methanol, and re-concentrated 2×. Sodium hydroxide (2.5 N) was added to adjust the pH to 14. After stirring at room temperature for 16 h., hydrochloric acid (1 N) was to adjust the pH to 1. This dilute mixture was concentrated until a white solid appeared which was collected by filtration (95 Mg). ¹H NMR (DMSO-d₆/300 MHz) 9.79 (brs, 1H), 7.85 (brs, 2H), 7.57 (d, 1H), 6.77 (m, 2H), 4.58 (m, 2H), 3.95 (s, 2H), 2.82 (m, 2H), 2.10 (m, 2H). ESHRMS m/z 306.090 (M+H, C₁₄H₁₅N₃O₃S requires m.w. 306.091).

### EXAMPLE 5

This example illustrates the production of 2-(3-aminopropyl)-8-(sec-butylamino)-7-hydroxy-4,5-dihydro-2H-benzo[g]indazole-3-carboxylic acid.

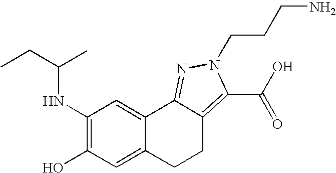

Step 1: The Preparation of Methyl 7-methoxy-4,5-dihydro-2H-benzo[g]indazole-3-carboxylate.

6-methoxytetralone (0.5 mol, 89.12 g) was placed in a 3-neck 5 L roundbottom flask equipped with an overhead stirrer, under nitrogen. Ether (1 L) was added followed by dimethyloxylate (0.55 mol, 64.95 g). The mixture was stirred for 10 min. when sodium methoxide (25%/methanol, 0.55 mol, 118.84 g) was slowly added. Ether (500 mL) was used when rinsing reagents into the flask. The mixture was stirred for 1 h., stirring was stopped for 16 h., and continued for an additional 4 h. when a greenish solid was collected by filtration and air-dried. A stream of nitrogen was passed through the flask to remove the remaining ether. The previously filtered green solid material was returned to the flask using methanol (200 mL) to rinse the filter. Methanol (800 mL) was added followed by methanesulfonic acid (0.55 mol, 52.86 g) and hydrazine mono hydrochloride (0.55 mol, 37.68 g) with a mild exotherm. The mixture was then heated to reflux with stirring for 3 h. After standing for 2 days at room temperature, sodium hydroxide (2.5 N, 200 mL) and water (200 mL) were added slowly. A light pink solid was collected by filtration and dried in vacuo at 80° (117.5 g). ¹H NMR (DMSO-d₆/400 MHz) 13.8 (brs, 1H), 7.6 (dd, 1H), 6.8 (m, 2H), 3.82 (s, 3H), 3.77 (s, 3H), 2.86 (m, 4H). ESHRMS m/z 259.105 (M+H, C₁₄H₁₄N₂O₃ requires 259.108).

Step 2: The Preparation of Methyl 7-methoxy-8-nitro-4,5-dihydro-2H-benzo[g]indazole-3-carboxylate.

Nitric acid (70%, 300 mL) was cooled to −30° and methyl 7-methoxy-4,5-dihydro-2H-benzo[g]indazole-3-carboxylate (0.1 mol, 25.8 g) was added in portions over 10 min. The mixture was stirred at 0° for 2 h. and poured into a mixture of ice (1.3 L) and ethyl acetate (600 mL). After stirring for 1 h., a yellow solid was collected by filtration. The filtrate was extracted with ethyl acetate (3×150 mL), Combined, washed with brine 2×, dried over magnesium sulfate, and concentrated to 300 mL. Hexane (100 mL) was added and the mixture was allowed to stand 16 h. A yellow solid was collected and combined with the previously obtained solid material (28.3 g).

¹H NMR (acetone-d₆/300 MHz) 8.25 (s, 1H), 7.38 (s, 1H), 4.05 (s, 3H), 3.94 (s, 3H), 3.11 (m, 4H). ESHRMS m/z 304.095 (M+H, C₁₄H₁₃N₃O₅ requires 304.093).

Step 3: The Preparation of Methyl 2-{3-[(tert-butoxycarbonyl) amino]propyl}-7-methoxy-8-nitro-4,5-dihydro-2H-benzo[g]indazole-3-carboxylate.

Methyl 7-methoxy-8-nitro-4,5-dihydro-2H-benzo[g]indazole-3-carboxylate (20 mmol, 6.06 g) and N,N′-dimethylformamide (100 mL) were cooled to 0° under nitrogen. Lithium t-butoxide (1.0 N/tetrahydrofuran, 40 mmol, 40 mL) was slowly added. After 30 min., tert-butyl 3-bromopropylcarbamate (16 mmol, 3.81 g) was added and the mixture was stirred at room temperature for 16 h. Hydrochloric acid (1 N, 50 mL) was added and the mixture was extracted with ethyl acetate (3×50 mL). The combined organics were washed with brine 2×, dried over magnesium sulfate, and concentrated. Purification was by flash column chromatography eluting with ethyl acetate/hexanes. The appropriate fractions were concentrated to a yellow solid (3.65 g). ¹H NMR (acetone-d₆/300 MHz) 8.22 (s, 1H), 7.35 (s, 1H), 6.06 (brs, 1H), 4.67 (t, 2H), 4.05 (s, 3H), 3.97 (s, 3H), 3.17 (dd, 2H), 3.09 (m, 4H), 2.0 (m, 2H), 1.44 (s, 9H). ESHRMS m/z 461.201 (M+H, C₂₂H₂₈N₄O₇ requires 461.203).

Step 4: The Preparation of Methyl 8-amino-2-{3-[(tert-butoxycarbonyl) amino]propyl}-7-methoxy-4,5-dihydro-2H-benzo[g]indazole-3-carboxylate.

Methyl 2-{3-[(tert-butoxycarbonyl)amino]propyl}-7-methoxy-8-nitro-4,5-dihydro-2H-benzo[g]indazole-3-carboxylate (3.55 g), methanol (50 mL), and palladium on carbon (10%, 200 Mg) were placed in a Parr shaker apparatus. The mixture was pressurized to 60 psi of hydrogen gas and agitated for 16 h. After the catalyst was removed by filtration, the mixture was concentrated to a pink/purple solid (2.3 g). ¹H NMR (acetone-d₆/300 MHz) 7.21 (s, 1H), 6.78 (s, 1H), 6.0 (brs, 1H), 4.61 (t, 2H), 3.94 (s, 3H), 3.88 (s, 3H), 3.15 (dd, 2H), 2.86 (m, 4H), 2.84 (brs, 2H), 2.05 (m, 2H), 1.44 (s, 9H). ESHRMS m/z 431.229 (M+H, C₂₂H₃₀N₄O₅ requires 431.229).

Step 5: The Preparation of Methyl 2-{3-[(tert-butoxycarbonyl)amino]propyl}-8-(sec-butylamino)-7-methoxy-4,5-dihydro-2H-benzo[g]indazole-3-carboxylate.

Methyl 8-amino-2-{3-[(tert-butoxycarbonyl)amino]propyl}-7-methoxy-4,5-dihydro-2H-benzo[g]indazole-3-carboxylate (1 mmol, 430 Mg), dichloromethane (25 mL), 2-butanone (1.2 mmol, 86 Mg), and acetic acid (2 mL) were stirred at room temperature under nitrogen. Sodium triacetoxyborohydride (2 mmol, 430 Mg) was added and the mixture was allowed to stir for 3 h. After methanol (10 mL) was added, the mixture was concentrated to 5 mL. Purification was by reverse phase HPLC. The appropriate fractions were combined and extracted with ethyl acetate. The combined organic extracts were washed with saturated aqueous sodium bicarbonate solution and brine. After drying over magnesium sulfate, the mixture was concentrated to a brown oil (400 Mg). ¹H NMR (acetone-d₆/300 MHz) 7.09 (s, 1H), 6.78 (s, 1H), 6.0 (brs, 1H), 4.63 (t, 2H), 3.95 (s, 3H), 3.88 (s, 3H), 3.53 (m, 1H), 3.16 (m, 4H), 3.00-2.82 (m, 4H), 2.1 (m, 2H), 1.64 (M, 2H), 1.45 (s, 9H), 1.23 (d, 3H), 1.01 (t, 3H). ESHRMS m/z 487.288 (M+H, C₂₆H₃₈N₄O₅ requires 487.291).

Step 6: The Preparation of 2-(3-aminopropyl)-8-(sec-butylamino)-7-hydroxy-4,5-dihydro-2H-benzo[g]indazole-3-carboxylic Acid.

Methyl 2-{3-[(tert-butoxycarbonyl)amino]propyl}-8-(sec-butylamino)-7-methoxy-4,5-dihydro-2H-benzo[g]indazole-3-carboxylate (300 Mg) and dichloromethane (5 mL) were stirred at −78° under nitrogen. Boron tribromide (1.0 M/dichloromethane, 10 mL) was added dropwise. The mixture was stirred at room temperature 16 h. Water and methanol (1:5) was added dropwise to a volume of 30 mL. The mixture was concentrated to 10 mL and filtered. The filtrate was purified by reverse phase HPLC. The appropriate fractions were concentrated to produce the trifluoroacetic acid salt of the desired product, which was triturated with acetonitrile to produce a white solid (95 Mg). ¹H NMR D₂O/300 MHz) 7.54 (s, 1H), 6.91 (s, 1H), 4.52 (t, 2H), 3.57 (m, 1H), 2.92-2.78 (m, 6H), 2.11 (q, 2H), 1.74 (m, 1H), 1.58 (m, 1H), 1.22 (d, 3H), 0.89 (t, 3H). ESHRMS m/z 359.208 (M+H, C₁₉H₂₆N₄O₃ requires 359.211).

### EXAMPLE 6

This example illustrates the production of 2-(3-aminopropyl)-8-(sec-butylamino)-7-methoxy-4,5-dihydro-2H-benzo[g]indazole-3-carboxylic acid.

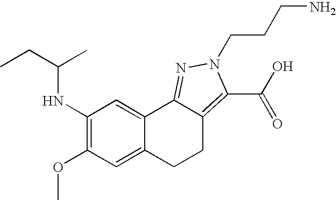

Methyl 2-{3-[(tert-butoxycarbonyl)amino]propyl}-8-(sec-butylamino)-7-methoxy-4,5-dihydro-2H-benzo[g]indazole-3-carboxylate (300 Mg) and dichloromethane (5 mL) were stirred at −78° under nitrogen. Boron tribromide (1.0 M/dichloromethane, 10 mL) was added dropwise. The mixture was stirred at room temperature 16 h. Water and methanol (1:5) was added dropwise to a volume of 30 mL. The mixture was concentrated to 10 mL and filtered. The filtrate was purified by reverse phase HPLC. The appropriate fractions were concentrated to produce the trifluoroacetic acid salt of the desired product as a brown oil (65 Mg). ¹H NMR D₂O/300 MHz) 7.48 (s, 1H), 6.83 (s, 1H), 4.24 (t, 2H), 3.75 (s, 3H), 3.57 (m, 1H), 2.89 (t, 2H), 2.68 (s, 4H), 2.06 (q, 2H), 1.72 (m, 1H), 1.54 (m, 1H), 1.20 (d, 3H), 0.88 (t, 3H). ESHRMS m/z 373.223 (M+H, C₂₀H₂₈N₄O₃ requires 373.222).

### EXAMPLE 7

This example illustrates the production of 2-(3-aminopropyl)-6-chloro-7-hydroxy-8-(3-nitrophenyl)-4,5-dihydro-2H-benzo[g]indazole-3-carboxylic acid.

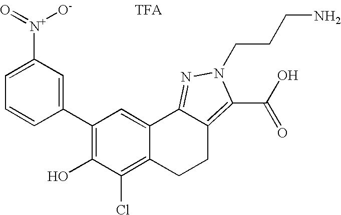

2-(3-aminopropyl)-7-hydroxy-8-(3-nitrophenyl)-4,5-dihydro-2H-benzo[g]indazole-3-carboxylic acid trifluoroacetate (0.2 mmol, 100 Mg) and acetic acid (5 mL) were stirred at room temperature. Chlorine (0.63 N/methanol, (0.2 mmol, 0.32 mL) was added and the mixture was allowed to stir at room temperature for three hours. After concentrating, the mixture was purified by reverse phase HPLC. The appropriate fractions were concentrated to a brown oil, the trifluoroacetic acid salt of the desired product (57 Mg). ¹H NMR (DMSO-d₆/400 MHz) 9.7 (bs, 1H), 8.35 (t, 1H), 8.2 (m, 1H), 7.97 (m, 1H), 7.73 (t, 1H), 7.68 (bs, 2H), 7.61, (s, 1H), 4.55 (t, 2H), 3.00 (m, 4H), 2.76 (m, 2H), 2.04 (m, 2H). ESHRMS m/z 443.113 (M+H, C₂₁H₁₉N₄O₅C[requires 443.112).

### EXAMPLE 8

This example illustrates the production of 2-amino-3-hydroxy-5,6,8,9,10,11-hexahydro-7H-benzo[g][1,4]diazepino[1,2-b]indazol-7-one trifluoroacetate.

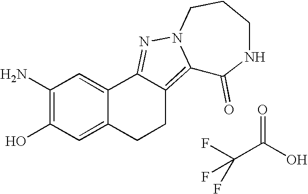

Step 1: The Preparation of 8-amino-2-(3-aminopropyl)-7-methoxy-4,5-dihydro-2H-benzo[g]indazole-3-carboxylate hydrochloride.

Methyl 8-amino-2-{3-[(tert-butoxycarbonyl)amino]propyl}-7-methoxy-4,5-dihydro-2H-benzo[g]indazole-3-carboxylate (3.7 mmol, 1.6 g), methanol (10 mL), and hydrochloric acid (4 N/dioxane, 30 mL) were stirred at room temperature for 2 h. The mixture was then concentrated and triturated with acetonitrile to produce a white solid. (0.1 g) was collected and characterized, the remaining material was used in the next step without further characterization. ¹H NMR (DMSO-d₆/300 MHz) 7.87 (bs, 4H), 7.56 (m, 1H), 7.11 (s, 1H), 4.58 (t, 2H), 3.91 (s, 3H), 3.90 (s, 3H), 2.95 (m, 4H), 2.85 (m, 2H), 2.10 (t, 2H). ESHRMS m/z 331.176 (M+H, C₁₇H₂₂N₄O₃ requires 331.176).

Step 2: The Preparation of 2-amino-3-hydroxy-5,6,8,9,10,11-hexahydro-7H-benzo[g][1,4]diazepino[1,2-b]indazol-7-one trifluoroacetate.

8-amino-2-(3-aminopropyl)-7-methoxy-4,5-dihydro-2H-benzo[g]indazole-3-carboxylate hydrochloride (1.2 mmol, 0.35 g and dichloromethane (20 mL) were stirred at −20° under nitrogen when boron tribromide (1.0 M/dichloromethane, 10 mL) was added drop wise. After stirring for 16 h at room temperature, methanol (10 mL) was added drop wise. The mixture was then concentrated and purified by reverse phase HPLC. The appropriate fractions were concentrated to produce the trifluoroacetic acid salt. ¹H NMR (DMSO-d₆/300 MHz) 10.5 (bs, 1H), 8.2 (bs, 1H), 7.50 (s, 1H), 6.87 (s, 1H), 4.39 (t, 2H), 3.18 (m, 2H), 2.82 (s, 4H), 2.13 (m, 2H). ESHRMS m/z 285.136 (M+H, C₁₅H₁₆N₄O₂ requires 285.135).

### EXAMPLE 9

This example illustrates the production of 7-hydroxy-1-tetralone.

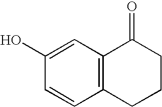

7-Methoxy-1-tetralone (25.0 g, 0.14 mol) and aluminum chloride (47.3 g, 0.355 mol) were refluxed in toluene (400 mL). Contents were allowed to cool, quenched with water (100 mL), extracted with EtOAc, dried over MgSO₄ and concentrated in vacuo leaving a tan solid (15.2 g). Recrystallization from EtOAc gave the desired as tan crystals, 13.9 g (61% yield). ¹H NMR (DMSO-d₆/300 MHz): 9.60 (s, 1H); 7.25-7.10 (m, 3H); 7.00-6.90 (m, 1H); 2.85-2.75 (m, 2H); 2.55-2.45 (m, 2H); 2.05-1.95 (m, 2H).

Anal. Calcd for C₁₀H₁₀O₂ (0.1H₂O): C, 73.24; H, 6.27. Found: C, 73.32; H, 5.88.

### EXAMPLE 10

This example illustrates the production of ethyl 6-bromo-7-[(2 methoxyethoxy)methoxy]-4,5-dihydro-2H-benzo[g]indazole-3-carboxylate.

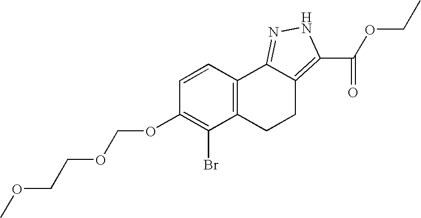

5-bromo-6-(methoxyethoxymethoxy)-1-tetralone [Bioorg & Med Chem Lett 7(12) 1573-1576 (1997)] was reacted according to the procedure for the production of ethyl (7-hydroxy-1-oxo-1,2,3,4-tetrahydronaphthalen-2-yl)(oxo)acetate to give the desired diketo-ester intermediate (86% yield). The diketo-ester intermediate was reacted according to the procedure for the production of ethyl 8-hydroxy-4,5-dihydro-2H-benzo[g]indazole-3-carboxylate to give the desired product as a solid (56% yield). FABHRMS m/z 425.0712, 427.0685 (M+H, C₁₈H₂₁BrN₂O₅ requires 425.0707, 427.0689). ¹H NMR (DMSO-d₆/300 MHz): 7.75 (d, 1H); 7.19 (d, 1H); 5.40 (s, 2H); 4.35 (q, 2H); 3.78-3.82 (m, 2H); 3.52-3.48 (m, 2H); 3.25 (s, 3H); 3.15-2.95 (m, 4H); 1.37 (t, 3H).

Anal. Calcd for C₁₈H₂₁BrN₂O₅ (0.4H₂O): C, 49.99; H, 5.08; N, 6.48. Found: C, 49.64; H, 4.66; N, 6.19.

### EXAMPLE 11

This example illustrates the production of 8-hydroxy-4,5-dihydro-2H-benzo[g]indazole-3-carboxylic acid.

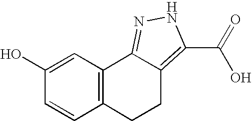

FABHRMS m/z 231.078 (M+H, C₁₂H₁₁N₂O₃ requires 231.0770).

### EXAMPLE 12

This example illustrates the production of 6-bromo-7-[(2-methoxyethoxy)methoxy]-4,5-dihydro-2H-benzo[g]indazole-3-carboxamide.

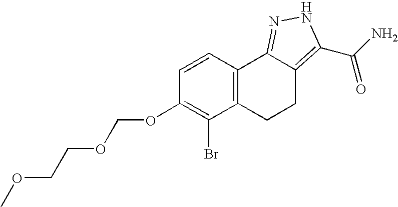

Ethyl 6-bromo-7-[(2-methoxyethoxy)methoxy]-4,5-dihydro-2H-benzo[g]indazole-3-carboxylate was reacted according to the procedure for the production of 8-hydroxy-4,5-dihydro-2H-benzo[g]indazole-3-carboxamide to give the desired product as a light amber solid (77% yield). FABHRMS m/z 396.0546, 398.0549 (M+H, C₁₆H₁₉BrN₃O₄ requires 396.0553, 398.0535).

Anal. Calcd for C₁₆H₁₈BrN₃O₄: C, 48.09; H, 4.39; N, 10.31. Found: C, 48.50; H, 4.58; N, 10.60.

### EXAMPLE 13

This example illustrates the production of 6-bromo-7-[(2-methoxyethoxy)methoxy]-4,5-dihydro-2H-benzo[g]indazole-3-carboxylic acid.

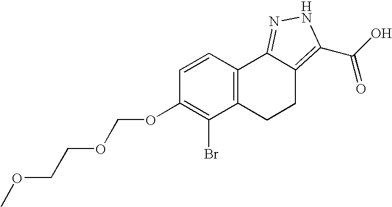

Ethyl 6-bromo-7-[(2-methoxyethoxy)methoxy]-4,5-dihydro-2H-benzo[g]indazole-3-carboxylate was reacted according to the procedure for that of 7-hydroxy-4,5-dihydro-2H-benzo[g]indazole-3-carboxylic acid to give the desired product as a solid (96% yield). FABHRMS m/z 397.0393, 399.0393 (M+H, C₁₆H₁₈BrN₂O₅ requires 397.0394, 399.0375).

Anal. Calcd for C₁₆H₁₈BrN₂O₅ (1.25H₂O): C, 45.78; H, 4.68; N, 6.67. Found: C, 45.71; H, 4.28; N, 6.62.

### EXAMPLE 14

This example illustrates the production of 6-bromo-7-hydroxy-4,5-dihydro-2H-benzo[g]indazole-3-carboxylic acid.

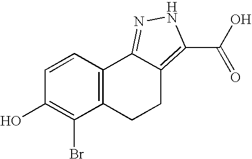

To 6-bromo-7-[(2-methoxyethoxy)methoxy]-4,5-dihydro-2H-benzo[g]indazole-3-carboxylic acid (1.4 g, 0.0035 mol) in CH₂Cl₂ (10 mL) was added TFA (5 mL) and stirred overnight. Contents were concentrated in vacuo and triturated with EtOAc/MeOH and filtered to give the desired product as an amber solid (88% yield). FABHRMS m/z 308.9845, 310.9813 (M+H, C₁₂H₁₀BrN₂O₃ requires 308.9869, 310.9850).

Anal. Calcd for C₁₂H₉BrN₂O₃ (1.1H₂O): C, 43.82; H, 3.43; N, 8.52. Found: C, 43.82; H, 3.16; N, 8.25.

### EXAMPLE 15

This example illustrates the production of ethyl 6-bromo-7-hydroxy-2H-benzo[g]indazole-3-carboxylate.

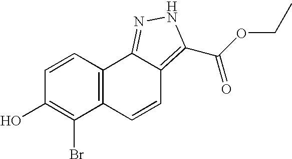

Ethyl 6-bromo-7-[(2-methoxyethoxy)methoxy]-4,5-dihydro-2H-benzo[g]indazole-3-carboxylate (5.0 g, 0.012 mol) and DDQ (2.7 g, 0.012 mol) were refluxed overnight. Contents were allowed to cool and the by-product was filtered. The filtrate was concentrated in vacuo and the residue was purified by silica gel chromatography eluting with 50% EtOAc/hexanes to give an amber solid, 5.2 g. The solid was triturated with CH₂Cl₂ and filtered to give the desired product as a light amber solid, 1.55 g (39% yield). FABHRMS m/z 335.0021, 337.0018 (M+H, C₁₄H₁₂BrN₂O₃ requires 335.0026, 337.0007). ¹H NMR (DMSO-d₆+5%TFA/300 MHz): 8.40 (d, 1H); 8.10 (d, 1H); 7.92 (d, 1H); 7.40 (d, 1H); 4.42 (q, 2H); 1.42 (t, 3H).

Anal. Calcd for C₁₄H₁₁BrN₂O₃(0.4H₂O): C, 49.12; H, 3.47; N, 8.18. Found: C, 49.10; H, 3.36; N, 7.68.

### EXAMPLE 16

This example illustrates the production of 6-bromo-7-hydroxy-2H-benzo[g]indazole-3-carboxylic acid

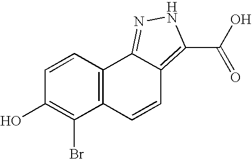

Ethyl 6-bromo-7-hydroxy-2H-benzo[g]indazole-3-carboxylate was reacted according to the procedure of 7-hydroxy-4,5-dihydro-2H-benzo[g]indazole-3-carboxylic acid to give the desired as a light amber solid (90% yield). FABHRMS m/z 306.9692, 308.9714 (M+H, C₁₂H₈BrN₂O₃ requires 306.9713, 308.9693). ¹H NMR (DMSO-d₆/300 MHz): 8.42 (d, 1H); 8.13 (d, 1H); 7.92 (d, 1H); 7.43 (d, 1H).

Anal. Calcd for C₁₂H₇BrN₂O₃ (2.0H₂O): C, 42.00; H, 3.23; N, 8.16. Found: C, 42.17; H, 2.96; N, 8.13.

### EXAMPLE 17

This example illustrates the production of ethyl 7-[(2-methoxyethoxy) methoxy]-4,5-dihydro-2H-benzo[g]indazole-3-carboxylate.

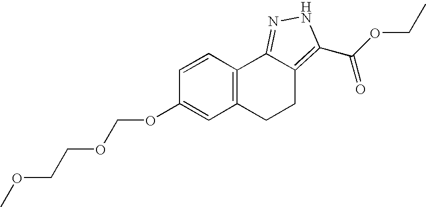

To 6-hydroxy-1-tetralone (26.0 g, 0.16 mol) in THF (200 mL) was added dropwise lithium t-butoxide (1M in THF, 170 mL). Contents were stirred a half hour and MEM chloride (19.4 mL, 0.17 mol) in THF (50 mL) was added dropwise. Contents were stirred overnight, partitioned between water and EtOAc. The EtOAc layer was dried over MgSO₄ and concentrated in vacuo leaving an oil, 39.9 g. The oil was reacted according to the procedure for the production of ethyl (7-hydroxy-1-oxo-1,2,3,4-tetrahydronaphthalen-2-yl)(oxo)acetate to give the desired diketo-ester intermediate as a yellow oil (59% yield). The diketo-ester intermediate was reacted according to the procedure for the production of ethyl 8-hydroxy-4,5-dihydro-2H-benzo[g]indazole-3-carboxylate to give the desired product as a light yellow solid (40% yield). FABHRMS m/z 347.1558 (M+H, C₁₈H₂₃N₂O₅ requires 347.1607). ¹H NMR (CDCl₃/300 MHz): 7.80 (d, 1H); 7.02 (d, 1H); 7.00 (s, 1H); 5.35 (s, 2H); 4.42 (q, 2H); 3.90-3.85 (m, 2H); 3.65-3.55 (m, 2H); 3.40 (s, 3H); 3.10-2.90 (m, 4H); 1.43 (t, 3H).

Anal. Calcd for C₁₈H₂₂N₂O₅: C, 62.42; H, 6.40; N, 8.09. Found: C, 62.20; H, 6.17; N, 7.92.

### EXAMPLE 18

This example illustrates the production of 7-[(2-methoxyethoxy)methoxy]-4,5-dihydro-2H-benzo[g]indazole-3-carboxylic acid.

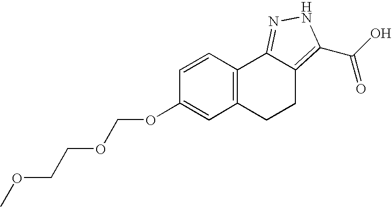

Ethyl 7-[(2-methoxyethoxy)methoxy]-4,5-dihydro-2H-benzo[g]indazole-3-carboxylate was reacted according to the procedure for the production of 7-hydroxy-4,5-dihydro-2H-benzo[g]indazole-3-carboxylic acid, to give the desired product (63% yield). FABHRMS m/z 319.1289 (M+H, C₁₆H₁₉N₂O₅ requires 319.1288).

Anal. Calcd for C₁₆H₁₈N₂O₅ (0.3H₂O): C, 59.36; H, 5.79; N, 8.65. Found: C, 59.35; H, 5.66; N, 8.55.

### EXAMPLE 19

This example illustrates the production of 7-[(2-methoxyethoxy)methoxy]-4,5-dihydro-2H-benzo[g]indazole-3-carboxamide.

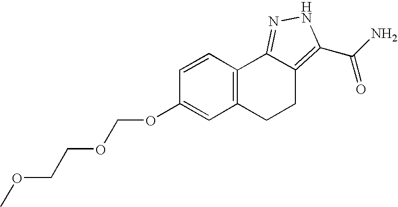

Ethyl 7-[(2-methoxyethoxy)methoxy]-4,5-dihydro-2H-benzo[g]indazole-3-carboxylate was reacted according to the procedure for the production of 8-hydroxy-4,5-dihydro-2H-benzo[g]indazole-3-carboxamide to give the desired product (81% yield). FABHRMS m/z 318.1439 (M+H, C₁₆H₂₀N₃O₄ requires 318.1448).

Anal. Calcd for C₁₆H₁₉N₃O₄ (0.7H₂O): C, 58.24; H, 6.23; N, 12.73. Found: C, 58.05; H, 6.35; N, 12.55.

### EXAMPLE 20

This example illustrates the production of ethyl 2-allyl-7-[(2-methoxyethoxy)methoxy]-4,5-dihydro-2H-benzo[g]indazole-3-carboxylate.

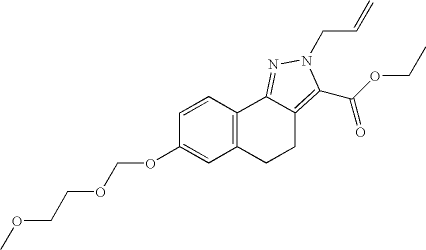

Ethyl 7-[(2-methoxyethoxy)methoxy]-4,5-dihydro-2H-benzo[g]indazole-3-carboxylate and allyl bromide were reacted according to the procedure for the production of ethyl 2-(3-aminopropyl)-2,4,5,6-tetrahydropyrazolo[3,4-e]indazole-3-carboxylate dihydrochloride to give the desired product after silica gel chromatography, eluting with 20% EtOAc/hexanes, as a colorless oil (70% yield). FABHRMS m/z 387.1910 (M+H, C₂₁H₂₇N₂O₅ requires 387.1920).

Anal. Calcd for C₂₁H₂₆N₂O₅: C, 65.27; H, 6.78; N, 7.25. Found: C, 66.13; H, 6.49; N, 7.42.

### EXAMPLE 21

This example illustrates the production of 2-allyl-7-[(2-methoxyethoxy)methoxy]-4,5-dihydro-2H-benzo[g]indazole-3-carboxamide.

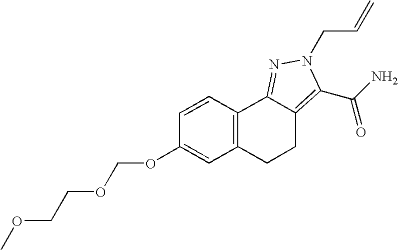

Ethyl 2-allyl-7-[(2-methoxyethoxy)methoxy]-4,5-dihydro-2H-benzo[g]indazole-3-carboxylate was reacted according to the procedure for the production of 8-hydroxy-4,5-dihydro-2H-benzo[g]indazole-3-carboxamide to give the desired as a white solid (40% yield). FABHRMS m/z 358.1756 (M+H, C₁₉H₂₄N₃O₄ requires 358.1761).

Anal. Calcd for C₁₉H₂₃N₃O₄: C, 63.85; H, 6.49; N, 11.76. Found: C, 63.58; H, 6.34; N, 11.65.

### EXAMPLE 22

This example illustrates the production of 2-allyl-7-[(2-methoxyethoxy) methoxy]-4,5-dihydro-2H-benzo[g]indazole-3-carboxylic acid.

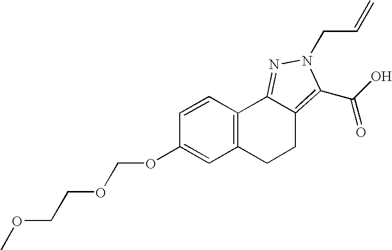

Ethyl 2-allyl-7-[(2-methoxyethoxy)methoxy]-4,5-dihydro-2H-benzo[g]indazole-3-carboxylate was reacted according to the procedure for the production of 7-hydroxy-4,5-dihydro-2H-benzo[g]indazole-3-carboxylic acid to give the desired as a white solid (62% yield). FABHRMS m/z 359.1619 (M+H, C₁₉H₂₃N₂O₅ requires 359.1601).

Anal. Calcd for C₁₉H₂₂N₂O₅ (0.2H₂O): C, 63.04; H, 6.24; N, 7.74. Found: C, 62.96; H, 5.79; N, 7.88.

### EXAMPLE 23

This example illustrates the production of 2-allyl-7-hydroxy-4,5-dihydro-2H-benzo[g]indazole-3-carboxylic acid.

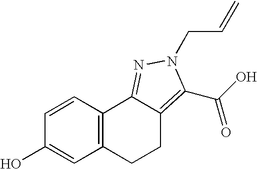

2-allyl-7-[(2-methoxyethoxy)methoxy]-4,5-dihydro-2H-benzo[g]indazole-3-carboxylic acid was reacted according to the procedure for the production of 6-bromo-7-hydroxy-4,5-dihydro-2H-benzo[g]indazole-3-carboxylic acid to give the desired product as an off-white solid (55% yield). FABHRMS m/z 271.1055 (M+H, C₁₅H₁₅N₂O₃ requires 271.1077). ¹H NMR (DMSO-d₆/300 MHz): 9.42 (br s, 1H); 7.50 (d, 2H); 6.65 (s, 2H); 6.10-5.96 (m, 1H); 5.204.95 (m, 4H); 3.00-2.80 (m, 4H).

Anal. Calcd for C₁₅H₁₄N₂O₃ (1.0H₂O): C, 62.49; H, 5.59; N, 9.72. Found: C, 62.79; H, 5.62; N, 9.65.

### EXAMPLE 24

This example illustrates the production of ethyl (7-hydroxy-1-oxo-1,2,3,4-tetrahydronaphthalen-2-yl)(oxo)acetate.

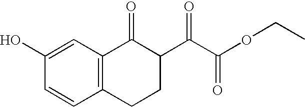

To 7-hydroxy-3,4-dihydronaphthalen-1 (2H)-one (13.9 g, 0.086 mol) and diethyl oxalate (27.2 mL, 0.2 mol) in ether (200 mL) was added dropwise lithium bis(trimethylsilyl)amide (1M in THF, 200 mL). Contents were stirred overnight and a solid was filtered. The solid was dissolved in water, which was made acidic to pH 2 with 1N HCl, precipitating a solid, which was filtered. Recrystallization from EtOAc/hexanes gave the desired as yellow crystals, 13.35 g (59% yield). FABHRMS m/z 263.0947 (M+H, C₁₄H₁₅O₅ requires 263.0919). ¹H NMR (CDCl₃/300 MHz): 7.42 (s, 1H); 7.15-7.06 (m, 1H); 7.03-6.98 (m, 1H); 5.80 (br s, 1H); 4.40 (q, 2H); 2.95-2.80 (m, 4H); 1.40 (t, 3H).

Anal. Calcd for C₁₄H₁₄O₅: C, 64.12; H, 5.38. Found: C, 64.04; H, 5.28

### EXAMPLE 25

This example illustrates the production of ethyl 8-hydroxy-4,5-dihydro-2H-benzo[g]indazole-3-carboxylate.

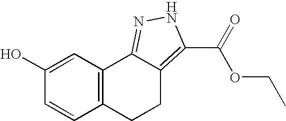

To ethyl (7-hydroxy-1-oxo-1,2,3,4-tetrahydronaphthalen-2-yl)(oxo)acetate (2.25 g, 0.009 mol) in glacial acetic acid (10 mL) was added dropwise hydrazine monohydrate (0.437 mL, 0.009 mol) in glacial acetic acid (2 mL). Contents were stirred overnight, diluted with water and a white solid was filtered, 1.8 g. The solid was triturated with acetonitrile and filtered to give the desired as a white solid, 1.5 g (68% yield). FABHRMS m/z 259.1091 (M+H, C₁₄H₁₅N₂O₃ requires 259.1083). ¹H NMR (DMSO-d₆/300 MHz): 9.43, 9.25 (s, 1H); 7.20-7.00 (m, 2H); 6.65-6.58 (m, 1H); 4.40-4.20 (m, 2H); 2.95-2.70 (m, 4H); 1.4-1.2 (m, 3H).

Anal. Calcd for C₁₄H₁₄N₂O₃: C, 65.11; H, 5.46; N, 10.85. Found: C, 64.87; H, 5.24; N, 10.85.

### EXAMPLE 26

This example illustrates the production of 8-Hydroxy-4,5-dihydro-2H-benzo[g]indazole-3-carboxamide.

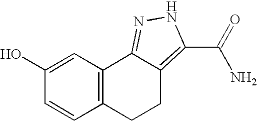

Ethyl 8-hydroxy-4,5-dihydro-2H-benzo[g]indazole-3-carboxylate (1.3 g, 0.005 mol), conc ammonium hydroxide (40 mL) and methanol (20 mL) were stirred in a stoppered flask for 48 hours. Contents were poured into a beaker to let the volatiles evaporate and a solid was filtered to give the desired as a light amber solid, 1.1 g, (96% yield). FABHRMS m/z 230.0935 (M+H, C₁₂H₁₂N₃O₂ requires 230.0930). ¹H NMR (DMSO-d₆/300 MHz): 9.40 (br s, 1H); 7.40 (br s, 1H); 7.20 (br s, 1H); 7.10-7.00 (m, 2H); 6.63-6.58 (m, 1H); 2.90-2.70 (m, 4H).

Anal. Calcd for C₁₂H₁₁N₃O₂ (1H₂O): C, 58.29; H, 5.30; N, 16.99. Found: C, 58.22; H, 5.54; N, 17.06.

### EXAMPLE 27

This example illustrates the production of ethyl 1-[4-(aminosulfonyl) phenyl]-8-hydroxy-4,5-dihydro-1H-benzo[g]indazole-3-carboxylate.

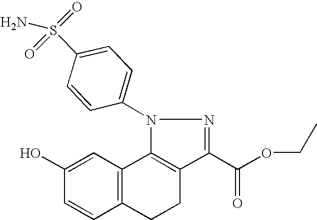

Ethyl 1-[4-(aminosulfonyl)phenyl]-8-hydroxy-4,5-dihydro-1H-benzo[g]indazole-3-carboxylate was prepared according to the procedure of ethyl 8-hydroxy-4,5-dihydro-2H-benzo[g]indazole-3-carboxylate, using 4-aminosulfonylphenyl hydrazine at 55° C. FABHRMS m/z 414.1143 (M+H, C₂₀H₂₀N₃O₅S requires 414.1124).

Anal. Calcd for C₂₀H₁₉N₃O₅S (0.2H₂O): C, 57.60; H, 4.69; N, 10.08. Found: C, 57.69; H, 4.85; N, 10.06.

### EXAMPLE 28

This example illustrates the production of 1-[4(Aminosulfonyl)phenyl]-8-hydroxy-4,5-dihydro-1H-benzo[g]indazole-3-carboxamide.

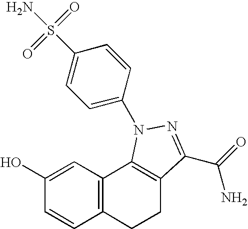

1-[4-(aminosulfonyl)phenyl]-8-hydroxy-4,5-dihydro-1H-benzo[g]indazole-3-carboxamide was prepared according to the procedure for the production of 8-hydroxy-4,5-dihydro-2H-benzo[g]indazole-3-carboxamide to give the desired as a white solid (88% yield). FABHRMS m/z 385.0985 (M+H, C₁₈H₁₇N₄O₄S requires 385.0971).

Anal. Calcd for C₁₈H₁₆N₄O₄S (0.3H₂O): C, 55.46; H, 4.29; N, 14.37. Found: C, 55.27; H, 4.37; N, 14.29.

### EXAMPLE 29

This example illustrates the production of ethyl (6-hydroxy-1-oxo-1,2,3,4-tetrahydronaphthalen-2-yl)(oxo)acetate.

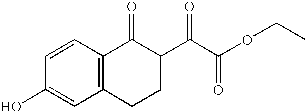

Ethyl (6-hydroxy-1-oxo-1,2,3,4-tetrahydronaphthalen-2-yl)(oxo)acetate was prepared according to the procedure for the production of ethyl (7-hydroxy-1-oxo-1,2,3,4-tetrahydronaphthalen-2-yl)(oxo)acetate to give the desired as a yellow solid (43% yield). FABHRMS m/z 263.0919 (M+H, C₁₄H₁₅O₅ requires 263.0925). ¹H NMR (CDCl₃/300 MHz): 8.02-7.95 (m, 1H); 7.30-7.20 (m, 1H); 6.85-6.70 (m, 1H); 6.65 (s, 1H); 5.40 (br s, 1H); 4.35-4.25 (m, 2H); 3.00-2.80 (m, 4H); 1.45-1.35 (t, 3H).

Anal. Calcd for C₁₄H₁₄O₅: C, 64.12; H, 5.38. Found: C, 63.79; H, 5.35.

### EXAMPLE 30

This example illustrates the production of ethyl 7-hydroxy-4,5-dihydro-2H-benzo[g]indazole-3-carboxylate.

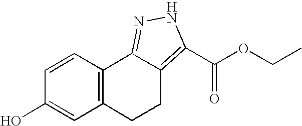

Ethyl 7-hydroxy-4,5-dihydro-2H-benzo[g]indazole-3-carboxylate was prepared according to the procedure of ethyl 8-hydroxy-4,5-dihydro-2H-benzo[g]indazole-3-carboxylate to give the desired product as a yellow solid (75% yield). FABHRMS m/z 259.1059 (M+H, C₁₄H₁₅N₂O₃ requires 259.1083). ¹H NMR (DMSO-d₆+5%TFA/300 MHz): 7.50 (d, 1H); 6.70-6.60 (m, 2H); 4.25 (q, 2H); 2.95-2.75 (m, 4H); 1.30 (t, 3H).

Anal. Calcd for C₁₄H₁₄N₂O₃ (0.85H₂O): C, 61.46; H, 5.78; N, 10.24. Found: C, 61.56; H, 5.70; N, 9.88.

### EXAMPLE 31

This example illustrates the production of 7-hydroxy-4,5-dihydro-2H-benzo[g]indazole-3-carboxylic acid.

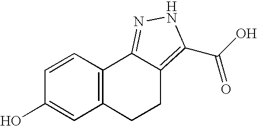

7-hydroxy-4,5-dihydro-2H-benzo[g]indazole-3-carboxylic acid (717 mg, 0.003 mol), aqueous sodium hydroxide (2.5N, 4 mL), water (5 mL) and methanol (5 mL) were stirred overnight. 1N HCl was added to pH 2, precipitating a solid, which was filtered to give the desired product as a white solid of 575 mg (89% yield). FABHRMS m/z 231.0773 (M+H, C₁₂H₁₁N₂O₃ requires 231.0770). ¹H NMR (DMSO-d₆+5%TFA/300 MHz): 7.55 (d, 1H); 6.75-6.65 (m, 2H); 2.90-2.75 (m, 4H).

Anal. Calcd for C₁₂H₁₀N₂O₃ (0.9H₂O): C, 58.49; H, 4.83; N, 11.37. Found: C, 58.69; H, 4.87; N, 11.49.

### EXAMPLE 32

This example illustrates the production of 7-hydroxy-4,5-dihydro-2H-benzo[g]indazole-3-carboxamide.

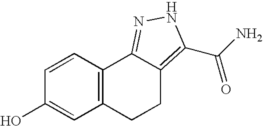

7-hydroxy-4,5-dihydro-2H-benzo[g]indazole-3-carboxylic acid was reacted according to the procedure for the production of 8-hydroxy-4,5-dihydro-2H-benzo[g]indazole-3-carboxamide to give the desired product as a tan solid (67% yield). FABHRMS m/z 230.0914 (M+H, C₁₂H₁₂N₃O₂ requires 230.0930). ¹H NMR (DMSO-d₆+TFA/300 MHz): 7.42 (d, 1H); 6.68-6.60 (m, 2H); 2.90-2.70 (m, 4H).

Anal. Calcd for C₁₂H₁₁N₃O₂ (0.2H₂O): C, 61.90; H, 4.94; N, 18.05. Found: C, 62.07; H, 4.87; N, 18.25.

### EXAMPLE 33

This example illustrates the production of ethyl 7-hydroxy-2-methyl-4,5-dihydro-2H-benzo[g]indazole-3-carboxylate.

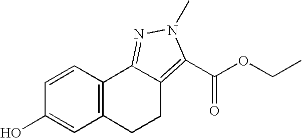

To ethyl (6-hydroxy-1-oxo-1,2,3,4-tetrahydronaphthalen-2-yl)(oxo)acetate (2.6 g, 0.01 mol) in glacial acetic acid (25 mL) was added dropwise methyl hydrazine (0.638 mL, 0.012 mol) in glacial acetic acid (5 mL). Contents were stirred overnight, diluted with water (100 mL) and a yellow solid (2.5 g) was filtered. Analysis of the solid by LC/MS shows two isomers (74:26). The solid was triturated with methanol (75 mL) and filtered to give the desired isomer as light yellow solid, 1.5 g (56% yield). The methanol filtrate was worked up below in Example 12 to obtain the other isomer. FABHRMS m/z 273.1217 (M+H, C₁₅H₁₆N₂O₃ requires 273.1239).

Anal. Calcd for C₁₅H₁₆N₂O₃: C, 66.16; H, 5.92; N, 10.29. Found: C, 65.97; H, 5.78; N, 10.15.

### EXAMPLE 34

This example illustrates the production of 7-hydroxy-2-methyl-4,5-dihydro-2H-benzo[g]indazole-3-carboxamide.

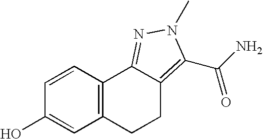

7-hydroxy-2-methyl-4,5-dihydro-2H-benzo[g]indazole-3-carboxamide was prepared according to the procedure for the production of 8-hydroxy-4,5-dihydro-2H-benzo[g]indazole-3-carboxamide FABHRMS m/z 244.1076 (M+H, C₁₃H₁₄N₃O₂ requires 244.1086).

Anal. Calcd for C₁₃H₁₃N₃O₂ (0.4H₂O): C, 62.34; H, 5.55; N, 16.78. Found: C, 62.22; H, 4.93; N, 6.74.

### EXAMPLE 35

This example illustrates the production of 7-hydroxy-2-methyl-4,5-dihydro-2H-benzo[g]indazole-3-carboxylic acid.

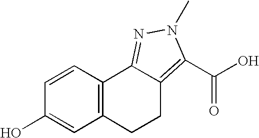

7-hydroxy-2-methyl-4,5-dihydro-2H-benzo[g]indazole-3-carboxylic acid was prepared according to the procedure of 7-hydroxy-4,5-dihydro-2H-benzo[g]indazole-3-carboxylic acid to give the desired as a white solid (98% yield). FABHRMS m/z 245.0906 (M+H, C₁₃H₁₃N₂O₃ requires 245.0926). ¹H NMR (DMSO-d₆/300 MHz): 9.70 (s, 1H); 7.55 (d, 1H); 6.80-6.70 (m, 2H); 2.95-2.75 (m, 4H).

Anal. Calcd for C₁₃H₁₂N₂O₃ (0.3H₂O): C, 62.54; H, 5.09; N, 11.22. Found: C, 62.37; H, 4.86; N, 11.19.

### EXAMPLE 36

This example illustrates the production of ethyl 7-hydroxy-1-methyl-4,5-dihydro-1H-benzo[g]indazole-3-carboxylate.

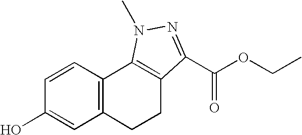

The methanol filtrate from ethyl 7-hydroxy-2-methyl-4,5-dihydro-2H-benzo[g]indazole-3-carboxylate was concentrated in vacuo. The residue was filtered through a pad of silica gel, eluting with 25% EtOAc/hexanes to give a solid (450 mg). The solid was recrystallized from EtOAc/MeOH gave the desired isomer as a white solid, 275 mg (10% yield). FABHRMS m/z 273.1216 (M+H, C₁₅H₁₇N₂O₃ requires 273.1239).

Anal. Calcd for C₁₅H₁₆N₂O₃: C, 66.16; H, 5.92; N, 10.29. Found: C, 65.78; H, 6.07; N, 10.30.

### EXAMPLE 37

This example illustrates the production of 7-hydroxy-1-methyl-4,5-dihydro-1H-benzo[g]indazole-3-carboxamide.

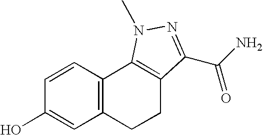

7-hydroxy-1-methyl-4,5-dihydro-1H-benzo[g]indazole-3-carboxamide was prepared according to the procedure for the production of 8-hydroxy-4,5-dihydro-2H-benzo[g]indazole-3-carboxamide to give the desired product as a light tan solid (59% yield). FABHRMS m/z 244.1084 (M+H, C₁₃H₁₄N₃O₂ requires 244.1086).

Anal. Calcd for C₁₃H₁₃N₃O₂ (H₂O): C, 59.68; H, 5.86; N, 16.34. Found: C, 59.64; H, 5.92; N, 16.35.

### EXAMPLE 38

This example illustrates the production of 7-hydroxy-N-methyl-4,5-dihydro-2H-benzo[g]indazole-3-carboxamide.

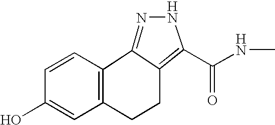

7-hydroxy-N-methyl-4,5-dihydro-2H-benzo[g]indazole-3-carboxamide was prepared according to the procedure for the production of 8-hydroxy-4,5-dihydro-2H-benzo[g]indazole-3-carboxamide, using 40% aqueous methyl amine to give the desired as an off-white solid (93% yield). FABHRMS m/z 244.1069 (M+H, C₁₃H₁₄N₃O₂ requires 244.1086).

Anal. Calcd for C₁₃H₁₃N₃O₂ (0.2H₂O): C, 63.25; H, 5.47; N, 17.02. Found: C, 63.02; H, 5.79; N, 17.01.

### EXAMPLE 39

This example illustrates the production of 2-amino-3-methoxy-5,6,8,9,10,11-hexahydro-7H-benzo[g][1,4]diazepino[1,2-b]indazol-7-one,

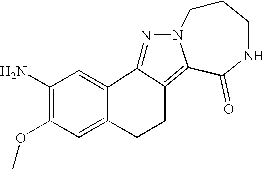

ESHRMS m/z 299.148 (M+H, C₁₆H₁₈N₄O₂ requires 299.150).

### EXAMPLE 40

This example illustrates the production of 6-methoxy-2,4-dihydroindeno[1,2-c]pyrazole-3-carboxylic acid,

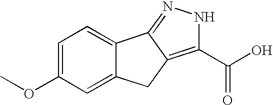

ESHRMS m/z 231.073 (M+H, C₁₂H₁₀N₂O₃ requires 231.076).

### EXAMPLE 41

This example illustrates the production of, methyl 2-{3-[(tert-butoxycarbonyl)amino]propyl}-6-methoxy-2,4-dihydroindeno[1,2-c]pyrazole-3-carboxylate

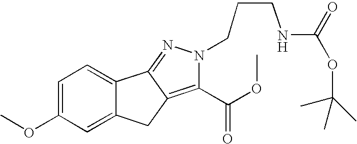

ESHRMS m/z 444.250 (M+H, C₂₄H₃₃N₃O₅ requires 444.249).

### EXAMPLE 42

This example illustrates the production of 7-methoxy-2,4-dihydroindeno[1,2-c]pyrazole-3-carboxylic acid.

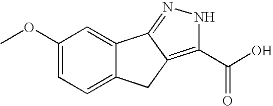

ESHRMS m/z 231.078 (M+H, C₁₂H₁₀N₂O₃ requires 231.076).

### EXAMPLE 43

This example illustrates the production of methyl 7-hydroxy-2,4-dihydroindeno[1,2-c]pyrazole-3-carboxylate.

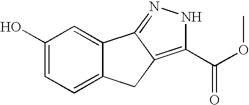

ESHRMS m/z 231.076 (M+H, C₁₂H₁₀N₂O₃ requires 231.076).

### EXAMPLE 44

This example illustrates the production of 7-hydroxy-2,4-dihydroindeno[1,2-c]pyrazole-3-carboxylic acid,

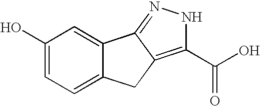

ESHRMS m/z 217.062 (M+H, C₁₁H₈N₂O₃ requires 217.061).

### EXAMPLE 45

This example illustrates the production of methyl 2-{3-[(tert-butoxycarbonyl)amino]propyl}-7-methoxy-2,4-dihydroindeno[1,2-c]pyrazole-3-carboxylate,

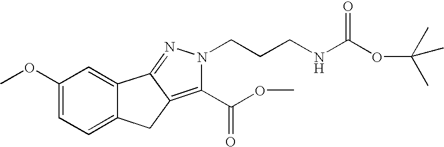

ESHRMS m/z 402.203 (M+H, C₂₁H₂₇N₃O₅ requires 402.202).

### EXAMPLE 46

This example illustrates the production of tert-butyl 2-{3-[(tert-butoxycarbonyl)amino]propyl}-7-methoxy-2,4-dihydroindeno[1,2-c]pyrazole-3-carboxylate,

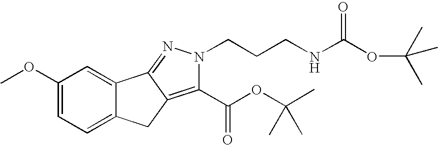

ESHRMS m/z 444.248 (M+H, C₂₄H₂₃N₃O₅ requires 444.249).

### EXAMPLE 47

This example illustrates the production of 7-methoxy-2,5-dihydroisothiochromeno[4,3-c]pyrazole-3-carboxylic acid,

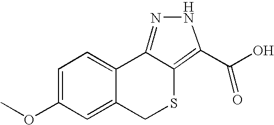

ESHRMS m/z 263.046 (M+H, C₁₂H₁₀N₂O₃S requires 263.048).

### EXAMPLE 48

This example illustrates the production of methyl 7-methoxy-5-[(4-methylphenyl)sulfonyl]-4,5-dihydro-2H-pyrazolo[4,3-c]quinoline-3-carboxylate,

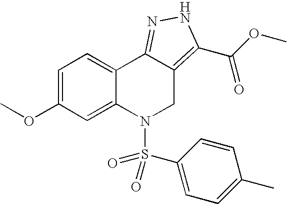

ESHRMS m/z 414.115 (M+H, C₂₀H₁₉N₃O₅S requires 414.112).

### EXAMPLE 49

This example illustrates the production of 2-bromo-3-methoxy-9-methyl-5,6,9,10-tetrahydrobenzo[g]pyrazino[1,2-b]indazol-7(8H)-one,

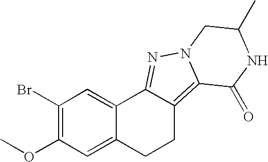

ESHRMS m/z 362.047 (M+H, C₁₆H₁₆N₃O₂Br requires 362.050).

### EXAMPLE 50

This example illustrates the production of 2-bromo-3-hydroxy-9-methyl-5,6,9,10-tetrahydrobenzo[g]pyrazino[1,2-b]indazol-7(8H)-one,

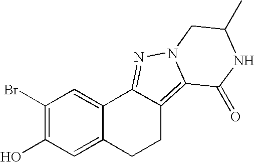

ESHRMS m/z 348.033 (M+H, C₁₅H₁₄N₃O₂Br requires 348.034).

### EXAMPLE 51

This example illustrates the production of methyl 2-{3-[(tert-butoxycarbonyl)amino]propyl}-8-(sec-butylamino)-7-methoxy-4,5-dihydro-2H-benzo[g]indazole-3-carboxylate,

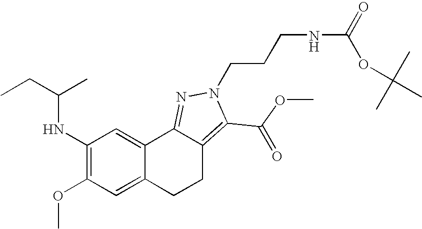

ESHRMS m/z 487.288 (M+H, C₂₆H₃₈N₄O₅ requires 487.291).

### EXAMPLE 52

This example illustrates the production of methyl 8-amino-2-(3-aminopropyl)-7-methoxy-4,5-dihydro-2H-benzo[g]indazole-3-carboxylate hydrochloride,

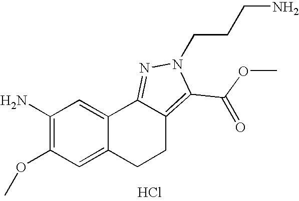

ESHRMS m/z 331.176 (M+H, C₁₇H₂₂N₄O₃ requires 331.176).

### EXAMPLE 53

This example illustrates the production of (7-methoxy-4,5-dihydro-2H-benzo[g]indazol-3-yl)methanol,

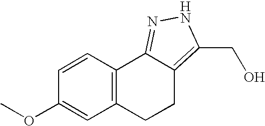

ESHRMS m/z 231.109 (M+H, C₁₃H₁₄N₂O₂ requires 231.113).

### EXAMPLE 54

This example illustrates the production of ethyl 7-hydroxy-1-(4-methoxyphenyl)-4,5-dihydro-1H-benzo[g]indazole-3-carboxylate.

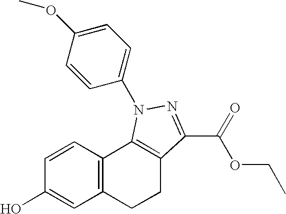

FABHRMS m/z 365.1514 (M+H, C₂₁H₂₁N₂O₄ requires 365.1496).

### EXAMPLE 55

This example illustrates the production of ethyl 7-hydroxy-1-phenyl-4,5-dihydro-1H-benzo[g]indazole-3-carboxylate,

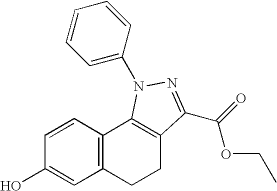

FABHRMS m/z 335.1384 (M+H, C₂₀H₁₉N₂O₃ requires 335.1396).

### EXAMPLE 56

This example illustrates the production of 6-bromo-7-hydroxy-2H-benzo[g]indazole-3-carboxamide,

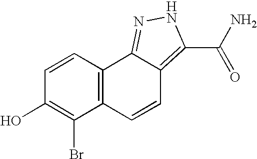

FABHRMS m/z 305.9880 (M+H, C₁₂H₉BrN₃O₂ requires 305.9873).

### EXAMPLE 57

This example illustrates the production of 7-hydroxy-1-(4-methoxyphenyl)-4,5-dihydro-1H-benzo[g]indazole-3-carboxamide,

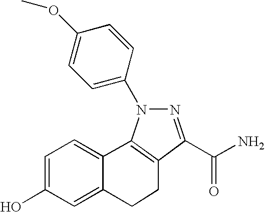

FABHRMS m/z 336.1347 (M+H, C₁₉H₁₈N₃O₃ requires 336.1348).

### EXAMPLE 58

This example illustrates the production of 7-hydroxy-1-phenyl-4,5-dihydro-1H-benzo[g]indazole-3-carboxamide,

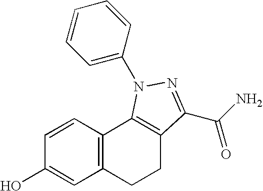

FABHRMS m/z 306.1248 (M+H, C₁₈H₁₆N₃O₂ requires 306.1243).

### EXAMPLE 59

This example illustrates the production of 7-hydroxy-N,N-dimethyl-4,5-dihydro-2H-benzo[g]indazole-3-carboxamide,

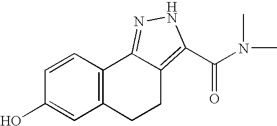

FABHRMS m/z 258.1268 (M+H, C₁₄H₁₆N₃O₂ requires 258.1243).

### EXAMPLE 60

This example illustrates the production of 7,8-dihydroxy-4,5-dihydro-2H-benzo[g]indazole-3-carboxylic acid.

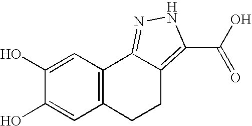

To boron tribromide (1.7 mL) in CH₂Cl₂ (25 mL) at −78° C. was added ethyl 7,8-dimethoxy-4,5-dihydro-2H-benzo[g]indazole-3-carboxylate (1.8 g, 0.006 mol) in CH₂Cl₂ (25 mL). Contents were stored at 0° C. overflight and poured into ice water. Contents were heated on a steam bath to evaporate the CH₂Cl₂ and filtered to give a red solid. Recrystallization from EtOAc/MeOH gave the diketo-acid as an orange solid, 727 mg (49% yield). FABHRMS m/z 249.0425 (M+H, C₁₂H₁₁O₆ requires 249.0399). ¹H NMR (DMSO-d₆+TFA/300 MHz): 7.30 (s, 1H); 6.60 (s, 1H); 2.65 (s, 4H). Anal. Calcd for C₁₂H₁₀O₆: C, 57.60; H, 4.03. Found: C, 57.63; H, 4.14. To the diketo-acid (500 mg, 0.002 mol) in glacial acetic acid (20 mL) was added hydrazine monohydrate (0.218 mL, 0.0045 mol). Contents were stirred overnight, added water and filtered a white solid, to give the desired product, 40 mg (8% yield). FABHRMS m/z 247.0729 (M+H, C₁₂H₁₁N₂O₄ requires 247.0719). ¹H NMR (DMSO-d₆+TFA/300 MHz): 7.10 (s, 1H); 6.63 (s, 1H); 2.90-2.81 (m, 2H); 2.78-2.65 (m, 2H).

Anal. Calcd for C₁₂H₁₀N₂O₄ (0.6H₂O): C, 56.08; H, 4.39; N, 10.90. Found: C, 56.03; H, 4.12; N, 10.73.

### EXAMPLE 61

This example illustrates the production of ethyl 6-bromo-7-hydroxy-4,5-dihydro-2H-benzo[g]indazole-3-carboxylate,

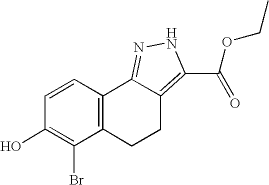

5-bromo-6-hydroxy-1-tetralone [Bioorg & Med Chem Lett 7(12) 1573-1576 (1997)] was reacted according to the procedure for the production of ethyl (7-hydroxy-1-oxo-1,2,3,4-tetrahydronaphthalen-2-yl)(oxo)acetate to give the desired diketo-ester intermediate (51% yield). The diketo-ester intermediate was reacted according to the procedure for the production of ethyl 8-hydroxy-4,5-dihydro-2H-benzo[g]indazole-3-carboxylate to give the desired product as yellow crystals (38% yield). FABHRMS m/z 337.0175, 339.0169 (M+H, C₁₄H₁₄BrN₂O₃ requires 337.0188, 339.0169). ¹H NMR (DMSO-d₆+TFA/300 MHz): 7.58 (d, 1H); 6.90 (d, 1H); 4.30 (q, 2H); 3.05-2.90 (m, 4H); 1.30 (t, 3H).

Anal. Calcd for C₁₄H₁₃BrN₂O₃: C, 49.87; H, 3.89; N, 8.31. Found: C, 51.44; H, 4.65; N, 7.28

### EXAMPLE 62

This example illustrates the production of 6-bromo-7-hydroxy-4,5-dihydro-2H-benzo[g]indazole-3-carboxamide,

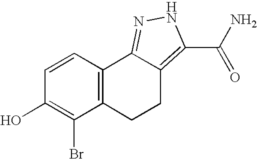

Ethyl 6-bromo-7-hydroxy-4,5-dihydro-2H-benzo[g]indazole-3-carboxylate was reacted according to the procedure for the production of 8-hydroxy-4,5-dihydro-2H-benzo[g]indazole-3-carboxamide to give the desired product as an off-white solid (62% yield). FABHRMS m/z 310.0020 (M+H, C₁₂H₁₁BrN₃O₂ requires 310.0010). ¹H NMR (DMSO-d₆+TFA/300 MHz): 7.55 (d, 1H); 6.92 (d, 1H); 3.10-2.90 (m, 4H).

Anal. Calcd for C₁₂H₁₀BrN₃O₂ (0.6H₂O): C, 45.19; H, 3.54; N, 13.17. Found: C, 45.00; H, 3.48; N, 13.09.

### EXAMPLE 63

This example illustrates the production of 7-hydroxy-N-(2-hydroxyethyl)-4,5-dihydro-2H-benzo[g]indazole-3-carboxamide,

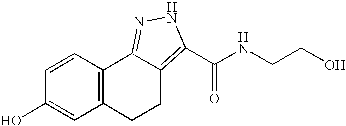

7-hydroxy-4,5-dihydro-2H-benzo[g]indazole-3-carboxylic acid (2.0 g, 0.0077 mol), ethanolamine (5 mL), water (5 mL) and methanol (5 mL) were stirred in a stoppered flask overnight. Contents were concentrated in vacuo and the residue was filtered through a pad of silica gel, eluting with EtOAc to give the desired as an oil. The oil was crystallized under methanol to give white crystals, 664 mg (32% yield). FABHRMS m/z 274.1152 (M+H, C₁₄H₁₆N₃O₃ requires 274.1186).

Anal. Calcd for C₁₄H₁₅N₃O₃: C, 61.53; H, 5.53; N, 15.38. Found: C, 61.45; H, 5.62; N, 15.35.

### EXAMPLE 64

This example illustrates the production of ethyl 2-(3-aminopropyl)-7-hydroxy-4,5-dihydro-2H-benzo[g]indazole-3-carboxylate dihydrochloride,

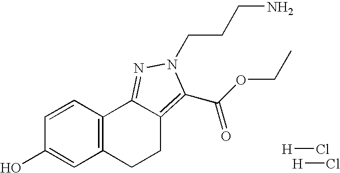

Ethyl 7-[(2-methoxyethoxy)methoxy]-4,5-dihydro-2H-benzo[g]indazole-3-carboxylate was reacted according to the procedure for the production of ethyl 2-(3-aminopropyl)-2,4,5,6-tetrahydropyrazolo[3,4-e]indazole-3-carboxylate dihydrochloride to give an oil, which was mixed with 4N HCl in dioxane and filtered to give the desired product as a white solid. FABHRMS m/z 316.1658 (M+H, C₁₇H₂₂N₃O₃ requires 316.1656). ¹H NMR (DMSO-d₆+5%TFA/300 MHz): 8.00 (br s, 1H); 7.80 (br s, 2H); 7.5 (d, 1H); 6.62 (s, 1H); 6.60 (d, 1H); 4.60-4.50 (m, 2H); 4.25 (q, 2H); 2.90-2.70 (m, 4H); 2.10-2.00 (m, 2H); 1.30 (t, 3H).

Anal. Calcd for C₁₇H₂₁N₃O₃(2H₂O): C, 48.12; H, 6.41; N, 9.90. Found: C, 48.00; H, 6.53; N, 10.89.

### EXAMPLE 65

This example illustrates the production of 2-{3-[(tert-butoxycarbonyl) amino]propyl}-7-hydroxy-4,5-dihydro-2H-benzo[g]indazole-3-carboxylic acid,

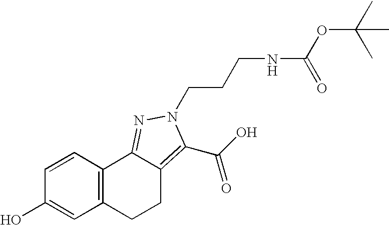

To ethyl 2-(3-aminopropyl)-7-hydroxy-4,5-dihydro-2H-benzo[g]indazole-3-carboxylate dihydrochloride (15.2 g, 0.04 mol) and di-t-butyl-dicarbonate (24.3 g) in CH₂Cl₂ was added triethylamine (10 mL) dropwise. Contents were stirred overnight, washed with water and the CH₂Cl₂ layer was dried over MgSO₄ and concentrated in vacuo leaving the intermediate Boc ester as a light yellow oil, 25.4 g. The intermediate Boc ester was reacted according to the procedure of 7-hydroxy-4,5-dihydro-2H-benzo[g]indazole-3-carboxylic acid to give the desired as a white solid (58% yield). FABHRMS m/z 388.1883 (M+H, C₂₀H₂₆N₃O₅ requires 388.1867). ¹H NMR (DMSO-d₆+5%TFA/300 MHz): 9.45 (s, 1H); 7.55 (d, 1H); 7.83 (br s, 1H); 6.70 (s, 1H); 6.65 (d, 1H); 4.49 (t, 2H); 3.00-2.80 (m, 6H); 2.00-1.80 (m, 2H); 1.40 (s, 9H).

Anal. Calcd for C₂₀H₂₅N₃O₅ (0.2H₂O): C, 61.43; H, 6.55; N, 10.75. Found: C, 61.35; H, 6.76; N, 10.47.

### EXAMPLE 66

This example illustrates the production of 2-(3-aminopropyl)-7-hydroxy-4,5-dihydro-2H-benzo[g]indazole-3-carboxylic acid dihydrochloride,

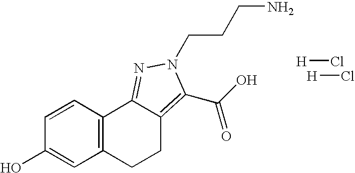

2-{3-[(tert-butoxycarbonyl)amino]propyl}-7-hydroxy-4,5-dihydro-2H-benzo[g]indazole-3-carboxylic acid (702 mg, 0.002 mol) and 4N HCl in dioxane (20 mL) were stirred overnight, diluted with EtOAc and filtered to give the desired product as a white solid, 608 mg (94% yield). FABHRMS m/z 288.1367 (M+H, C₁₅H₁₈N₃O₃ requires 288.1343). ¹H NMR (DMSO-d₆+5%TFA/300 MHz): 7.90 (br s, 3H); 7.52 (d, 1H); 6.70 (s, 1H); 6.65 (d, 1H); 4.60 (t, 2H); 3.00-2.80 (m, 4H); 2.10 (t, 2H).

Anal. Calcd for C₁₅H₁₇N₃O₃ (2.3 HCl): C, 48.54; H, 5.24; N, 11.32. Found: C, 48.53; H, 5.82; N, 11.78.

### EXAMPLE 67

This example illustrates the production of 7-hydroxy-1-methyl-4,5-dihydro-1H-benzo[g]indazole-3-carboxylic acid,

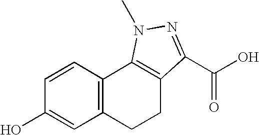

Ethyl 7-hydroxy-1-methyl-4,5-dihydro-1H-benzo[g]indazole-3-carboxylate was reacted according to the procedure of 7-hydroxy-4,5-dihydro-2H-benzo[g]indazole-3-carboxylic acid to give the desired product as a white solid (95% yield). FABHRMS m/z 245.0896 (M+H, C₁₃H₁₃N₂O₃ requires 245.0921). ¹H NMR (DMSO-d₆/300 MHz): 7.50 (d, 1H); 6.70 (s, 1H); 6.68 (d, 1H); 4.10 (s, 3H); 2.95-2.75 (m, 4H).

Anal. Calcd for C₁₃H₁₂N₂O₃ (1.3H₂O): C, 58.33; H, 5.50; N, 10.47. Found: C, 58.36; H, 5.09; N, 10.41.

### EXAMPLE 68

This example illustrates the production of ethyl 7-hydroxy-2H-benzo[g]indazole-3-carboxylate,

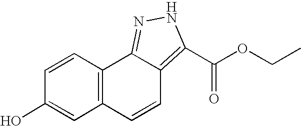

7-hydroxy-4,5-dihydro-2H-benzo[g]indazole-3-carboxylic acid (5.0 g, 0.02 mol) and DDQ (5.5 g, 0.025 mol) were stirred in dioxane (30 mL) for 72 hours. A solid was filtered (by-product) and the filtrate was concentrated in vacuo leaving an amber solid. The solid was recrystallized from methanol to give the desired product as a light amber solid, 246 mg. More desired product was recovered from the methanol filtrate, 1.1 g (26% total yield). FABHRMS m/z 257.0891 (M+H, C₁₄H₁₃N₂O₃ requires 257.0921). ¹H NMR (DMSO-d₆/300 MHz): 9.98 (s, 1H); 8.38 (d, 1H); 7.95 (d, 1H); 7.55 (d, 1H); 7.35 (s, 1H); 7.22 (d, 1H); 4.40 (q, 2H); 1.40 (t, 3H).

Anal. Calcd for C₁₄H₁₂N₂O₃ (0.7H₂O): C, 62.54; H, 5.02; N, 10.42. Found: C, 62.84; H, 4.69; N, 10.39.

### EXAMPLE 69

This example illustrates the production of 3-hydroxy-5,6,8,9,10,11-hexahydro-7H-benzo[g][1,4]diazepino[1,2-b]indazol-7-one,

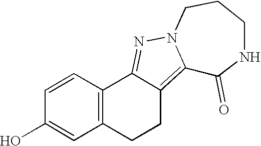

Ethyl 2-(3-aminopropyl)-7-hydroxy-4,5-dihydro-2H-benzo[g]indazole-3-carboxylate dihydrochloride was reacted according to the procedure for the production of 8-hydroxy-4,5-dihydro-2H-benzo[g]indazole-3-carboxamide to give the desired product as a light amber solid (46% yield). FABHRMS m/z 270.1209 (M+H, C₁₅H₁₆N₃O₂ requires 270.1237). ¹H NMR (DMSO-d₆+5%TFA/300 MHz): 8.19 (br s, 1H); 7.50 (d, 1H); 6.68 (s, 1H); 6.62 (d, 1H); 5.75 (s, 1H); 4.40 (t, 2H); 3.24-3.15 (m, 2H); 2.80 (s, 4H); 2.20-2.05 9 (m, 2H).

Anal. Calcd for C₁₅H₁₅N₃O₂ (HCl): C, 58.92; H, 5.27; N, 13.74. Found: C, 59.10; H, 5.18; N, 13.46.

### EXAMPLE 70

This example illustrates the production of 7-hydroxy-2H-benzo[g]indazole-3-carboxylic acid,

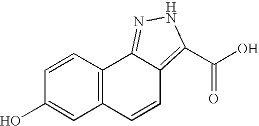

Ethyl 7-hydroxy-2H-benzo[g]indazole-3-carboxylate was reacted according to the procedure of 7-hydroxy-4,5-dihydro-2H-benzo[g]indazole-3-carboxylic acid to give the desired product as a white solid (95% yield). FABHRMS m/z 229.0616 (M+H, C₁₂H₉N₂O₃ requires 229.0608). ¹H NMR (DMSO/300 MHz): 9.95 (br s, 1H) 8.35 (d, 1H); 7.95 (d, 1H); 7.50 (d, 1H); 7.30 (s, 1H); 7.22 (d, 1H).

Anal. Calcd for C₁₂H₈N₂O₃ (H₂O): C, 58.54; H, 4.09; N, 11.38. Found: C, 58.07; H, 4.08; N, 11.32.

### EXAMPLE 71

This example illustrates the production of 7-hydroxy-2H-benzo[g]indazole-3-carboxamide,

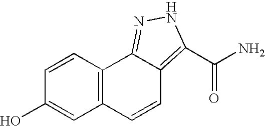

Ethyl 7-hydroxy-2H-benzo[g]indazole-3-carboxylate was reacted according to the procedure for the production of 8-hydroxy-4,5-dihydro-2H-benzo[g]indazole-3-carboxamide to give the desired product (62% yield). FABHRMS m/z 228.0786 (M+H, C₁₂H₁₀N₃O₂ requires 228.0768). ¹H NMR (DMSO-d₆+TFA/300 MHz): 8.32 (d, 1H); 8.03 (d, 1H); 7.40 (d, 1H); 7.28 (s, 1H); 7.20 (d, 1H).

Anal. Calcd for C₁₂H₉N₃O₂ (0.25H₂O): C, 62.20; H, 4.13; N, 18.13. Found: C, 62.67; H, 4.05; N, 17.69.

### EXAMPLE 72

This example illustrates the production of ethyl [6-(acetylamino)-1-oxo-1,2,3,4-tetrahydronaphthalen-2-yl](oxo)acetate,

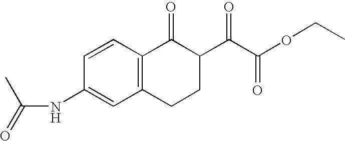

Ethyl [6-(acetylamino)-1-oxo-1,2,3,4-tetrahydronaphthalen-2-yl](oxo)acetate was prepared according to the procedure for the production of ethyl (7-hydroxy-1-oxo-1,2,3,4-tetrahydronaphthalen-2-yl)(oxo)acetate using 6-(acetylamino)-1-tetralone to give the desired product as yellow crystals (42% yield). FABHRMS m/z 304.1216 (M+H, C₁₆H₁₉NO₅ requires 304.1185).

Anal. Calcd for C₁₆H₁₇NO₅: C, 63.36; H, 5.65; N, 4.62. Found: C, 63.47; H, 5.46; N, 4.46.

### EXAMPLE 73

This example illustrates the production of ethyl 7-(acetylamino)-4,5-dihydro-2H-benzo[g]indazole-3-carboxylate,

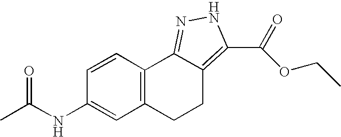

Ethyl [6-(acetylamino)-1-oxo-1,2,3,4-tetrahydronaphthalen-2-yl](oxo)acetate was reacted according to the procedure for ethyl 8-hydroxy-4,5-dihydro-2H-benzo[g]indazole-3-carboxylate to give the desired product as a yellow solid (41% yield). FABHRMS m/z 300.1347 (M+H, C₁₆H₁₈N₃O₃ requires 300.1348).

Anal. Calcd for C₁₆H₁₇N₃O₃ (0.5H₂O): C, 62.31; H, 5.55; N, 13.64. Found: C, 62.31; H, 5.55; N, 13.64.

### EXAMPLE 74

This example illustrates the production of 7-(acetylamino)-4,5-dihydro-2H-benzo[g]indazole-3-carboxamide,

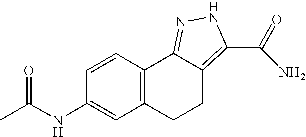

Ethyl 7-(acetylamino)-4,5-dihydro-2H-benzo[g]indazole-3-carboxylate was reacted according to the procedure for the production of 8-hydroxy-4,5-dihydro-2H-benzo[g]indazole-3-carboxamide to give the desired product as a white solid (76% yield). FABHRMS m/z 271.1208 (M+H, C₁₄H₁₅N₄O₂ requires 271.1195).

Anal. Calcd for C₁₄H₁₄N₄O₂: C, 62.21; H, 5.22; N, 20.73. Found: C, 62.27; H, 5.25; N, 20.62.

### EXAMPLE 75

This example illustrates the production of 7-amino-4,5-dihydro-2H-benzo[g]indazole-3-carboxamide,

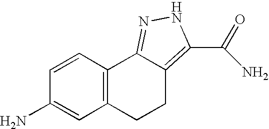

7-(acetylamino)-4,5-dihydro-2H-benzo[g]indazole-3-carboxamide (318 mg, 0.0012 mol) and 1N HCl (20 mL) were refluxed in MeOH (10 mL) overnight giving a mixture of amide and methyl ester. The contents were concentrated in vacuo and mixed with conc ammonium hydroxide (20 mL) and MeOH (5 mL), and stirred in a stoppered flask for 72 hours. Contents were poured into a beaker to let the volatiles evaporate and filtered to give the desired product as a light tan solid, 205 mg (75% yield). FABHRMS m/z 229.1106 (M+H, C₁₂H₁₃N₄O requires 229.1089).

Anal. Calcd for C₁₂H₁₂N₄O (0.25H₂O): C, 61.92; H, 5.41; N, 24.07. Found: C, 62.24; H, 5.2; N, 23.73.

### EXAMPLE 76

This example illustrates the production of 3-ethyl 7-methyl 4,5-dihydro-2H-benzo[g]indazole-3,7-dicarboxylate.

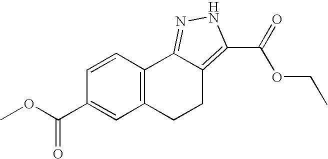

3-ethyl 7-methyl 4,5-dihydro-2H-benzo[g]indazole-3,7-dicarboxylate was prepared according to the procedures for the production for the production of ethyl (7-hydroxy-1-oxo-1,2,3,4-tetrahydronaphthalen-2-yl)(oxo)acetate and for the production of ethyl 8-hydroxy-4,5-dihydro-2H-benzo[g]indazole-3-carboxylate, starting with 6-carboxymethyltetralone [Tet. Lett 33(38) 5499 1992] to give the desired product as an off-white solid (47% yield). FABHRMS m/z 301.1192 (M+H, C₁₆H₁₇N₂O₄ requires 301.1188). ¹H NMR (CDCl₃/300 MHz): 8.20 (brs, 1H); 7.99 (s, 3H); 4.40 (q, 2H); 3.15-3.00 (m, 4H); 1.45 (t, 3H).

Anal. Calcd for C₁₆H₁₆N₂O₄: C, 63.99; H, 5.37; N, 9.33. Found: C, 63.73; H, 5.25; N, 9.29.

### EXAMPLE 77

This example illustrates the production of 4,5-dihydro-2H-benzo[g]indazole-3,7-dicarboxamide.

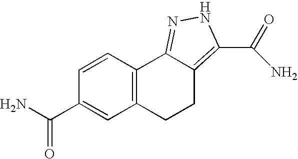

3-ethyl 7-methyl 4,5-dihydro-2H-benzo[g]indazole-3,7-dicarboxylate was reacted according to the procedure for the production of 8-hydroxy-4,5-dihydro-2H-benzo[g]indazole-3-carboxamide to give the desired product as a pale yellow solid (54% yield). FABHRMS m/z 257.1015 (M+H, C₁₃H₁₃N₄O₂ requires 257.1039). ¹H NMR (DMSO-d₆/300 MHz): 7.83-7.68 (m, 3H); 7.30 (br s, 2H); 2.95 (s, 4H).

Anal. Calcd for C₁₃H₁₃N₄O₂ (0.1H₂O): C, 60.51; H, 4.77; N, 21.71. Found: C, 60.56; H, 4.57; N, 21.35.

### EXAMPLE 78

This example illustrates the production of 7-(aminocarbonyl)-4,5-dihydro-2H-benzo[g]indazole-3-carboxylic acid.

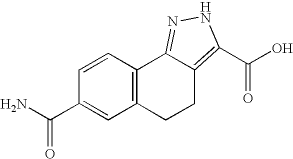

7-(aminocarbonyl)-4,5-dihydro-2H-benzo[g]indazole-3-carboxylic acid was a side product in the preparation of 4,5-dihydro-2H-benzo[g]indazole-3,7-dicarboxamide obtained as a second crop from the aqueous filtrate to give desired product as a white solid. FABHRMS m/z 258.0883 (M+H, C₁₃H₁₂N₃O₃ requires 258.0873).

Anal. Calcd for C₁₃H₁₁N₃O₃(0.7H₂O): C, 57.86; H, 4.63; N, 15.57. Found: C, 57.92; H, 4.71; N, 15.65.

### EXAMPLE 79

This example illustrates the production of 4,5-dihydro-2H-benzo[g]indazole-3,7-dicarboxylic acid.

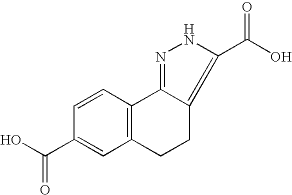

3-ethyl 7-methyl 4,5-dihydro-2H-benzo[g]indazole-3,7-dicarboxylate was reacted according to the procedure of that for the production of 7-hydroxy-4,5-dihydro-2H-benzo[g]indazole-3-carboxylic acid to give the desired product as a white solid (64% yield). FABHRMS m/z 259.0737 (M+H, C₁₃H₁₁N₂O₄ requires 259.0719).

Anal. Calcd for C₁₃H₁₀N₂O₄(0.55H₂O): C, 58.23; H, 4.17; N, 10.45. Found: C, 58.54; H, 4.68; N, 10.12.

### EXAMPLE 80

This example illustrates the production of ethyl (6,7-dimethoxy-1-oxo-1,2,3,4-tetrahydronaphthalen-2-yl)(oxo)acetate.

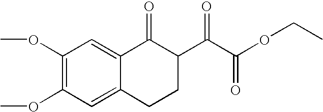

6,7-dimethoxy-1-tetralone was reacted according to the procedure for the production of ethyl (7-hydroxy-1-oxo-1,2,3,4-tetrahydronaphthalen-2-yl)(oxo)acetate to give the desired product as a yellow solid (86% yield). FABHRMS m/z 307.1185 (M+H, C₁₆H₁₉O₆ requires 307.1182). ¹H NMR (CDCl₃/300 MHz): 7.50 (s, 1H); 6.65 (s, 1H); 4.40 (q, 2H); 3.92 (s, 3H); 3.94 (s, 3H); 3.05-2.80 (m, 4H); 1.4 (t, 3H).

Anal. Calcd for C₁₆H₁₈O₆: C, 62.74; H, 5.92. Found: C, 62.54; H, 5.83.

### EXAMPLE 81

This example illustrates the production of ethyl 7,8-dimethoxy-4,5-dihydro-2H-benzo[g]indazole-3-carboxylate.

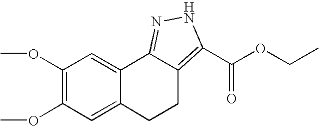

Ethyl 6,7-dimethoxy-1-oxo-1,2,3,4-tetrahydronaphthalen-2-yl](oxo)acetate was reacted according to the procedure for the production of ethyl 8-hydroxy-4,5-dihydro-2H-benzo[g]indazole-3-carboxylate to give the desired product as a light amber solid (67% yield). FABHRMS m/z 303.1334 (M+H, C₁₆H₁₉N₂O₄ requires 303.1345). ¹H NMR (CDCl₃/300 MHz): 7.40 (s, 1H); 6.78 (s, 1H); 4.40 (q, 2H); 3.95 (s, 3H); 3.90 (s, 3H); 3.05-2.85 (m, 4H); 1.40 (t, 3H).

Anal. Calcd for C₁₆H₁₈N₂O₄: C, 63.56; H, 6.00; N, 9.27. Found: C, 63.63; H, 5.97; N, 9.19.

### EXAMPLE 82

This example illustrates the production of 2-(3-Aminopropyl)-7-hydroxy-4-methyl-2H-benzo[g]indazole-3-carboxylic acid trifluoroacetate.

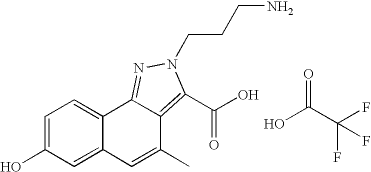

Step 1: Preparation of 2-methyl-7-methoxytetralone.

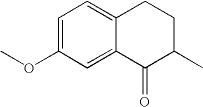

In a 1L 3-neck flask under Ar, lithium bis(TMS) amide (192 mL, 192 mmol of a 1.0 M solution in THF) was cooled to 0° C. 7-Methoxy tetralone (50.0 g, 284 mmol) dissolved in THF (200 mL) was added via addition funnel over one hour. Stirred at 0° C. for 30 minutes. In a 2L 3-neck flask, iodomethane (109 g, 768 mmol) was cooled to −78° C. The contents of the 1L flask were added to the 2L flask via cannula. Stirred at −78° C. for two hours. The reaction was quenched with NH4Cl (aq.). Extracted with ethyl acetate (2×300 mL). The organic phases were combined, washed with brine, dried over MgSO₄, filtered, and evaporated to yield a dark brown oil. Purified by flash column chromatography in 3% ethyl acetate/97% hexane. (30.25 g, 83%): ¹H NMR (CDCl₃/300 MHz) 7.52 (d, 1H), 7.14 (d, 1H), 7.05 (dd, 1H), 3.83 (s, 3H), 3.01-2.87 (m, 2H), 2.61-2.53 (m, 1H), 2.22-2.14 (m, 1H), 1.92-1.83 (m, 1H), 1.22 (d, 3H). ESHRMS m/z 191.1092 (M+H, C₁₂H₁₄O₂ requires 191.1067).

Step 2: Preparation of 2-ethyl-7-methoxy-1,2,3,4-tetrahydronaphthalene.

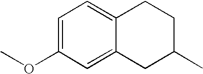

2-Methyl-7-methoxytetralone (29.14 g, 153 mmol) was dissolved in CH₂Cl₂ (150 mL). Triethylsilane (35.5 g, 306 mmol was added and the reaction was cooled to ⁰° C. Trifluoroacetic acid (175 mL, 1.53 mol) was slowly added via addition funnel. The ice bath was removed and the reaction stirred at room temperature overnight. Dichloromethane and trifluoroacetic acid were evaporated. Sodium bicarbonate (aq.) was added to consume any remaining trifluoroacetic acid. Extracted with dichloromethane (2×200 mL). The combined organic phases were washed with brine, dried over MgSO₄, filtered, and evaporated. Purified by flash column chromatography in hexane. (14.67 g, 54%): ¹H NMR (CDCl₃/300 MHz) 6.98 (d, 1H), 6.65 (dd, 1H), 6.59 (s, 1H), 3.76 (s, 3H), 2.82-2.75 (m, 3H), 2.42-2.33 (m, 1H), 1.87-1.76 (m, 2H), 1.42-1.33 (m, 1H), 1.04 (d, 3H). GCHRMS (m/z) 176.1189 (M+H C₁₂H₁₆O, requires 176.1201).

Step 3: Preparation of 6-methoxy-3-methyl-3,4-dihydronaphthalen-1 (2H)-one.

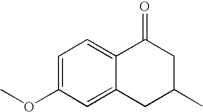

2-Ethyl-7-methoxy-1,2,3,4-tetrahydronaphthalene (10.8 g, 61.3 mmol) was dissolved in acetic acid (200 mL). Chromium trioxide (12.25 g, 122 mmol) was slowly added in small portions over two hours. The reaction mixture was heated to 90° C. for four hours and stirred at room temperature overnight. Acetic acid was evaporated. The remaining residue was neutralized with sodium bicarbonate (aq.). Extracted with ethyl acetate (2×200 mL). The combined organic phases were washed with brine, dried over MgSO₄, filtered, and evaporated. Purified by flash column chromatography in 10% ethyl acetate/90% hexane. The resulting solid was recrystallized from hexane (5.00 g 43%): mp 70.5-70.6° C. ¹H NMR (CDCl₃/300 MHz) 8.00 (d, 1H), 6.82 (dd, 1H), 6.70 (s, 1H), 3.85 (s, 3H), 2.95-2.89 (m, 1H), 2.71-2.61 (m, 2H), 2.30-2.19 (m, 2H), 1.13 (d, 3H). ESHRMS (m/z) 191.1073 (M+H, C₁₂H₁₄O₂ requires 191.1067).

Step 4: Preparation of Methyl (2Z)-hydroxy(6-methoxy-3-methyl-1-oxo-3,4-dihydronaphthalen-2(1H)-ylidene)ethanoate.

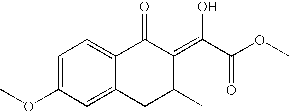

6-Methoxy-3-methyl-3,4-dihydronaphthalen-1 (2H)-one (3.00 g, 15.8 mmol) was dissolved in ether (50 mL). Dimethyl oxalate (2.05 g, 17.4 mmol) and sodium methoxide (3.98 mL, 3.76 g, 17.4 mmol of a 25%/wt. solution in methanol) were added. Stirred at room temperature overnight and added 1 N HCl to pH=1. The solution was transferred to a separatory funnel and the layers were separated. The aqueous phase was washed with ethyl acetate (2×100 mL). The combined organic phases were washed with brine (200 mL), dried over MgSO₄, filtered, and evaporated. The resulting brown solid was filtered through a plug of silica with 20% ethyl acetate/80% hexane to afford a dark yellow solid. (2.34 g, 54%) mp 89.0-89.6° C. ¹H NMR (CDCl₃/300 MHz) 16.13 (s, 1H), 7.98 (d, 1H), 6.87 (d, 1H), 6.72 (s, 1H), 3.92 (s, 3H), 3.88 (s, 3H), 3.52 (t, 1H), 3.15 (dd, 1H), 2.66-2.60 (m, 1H), 1.09 (d, 3H). ESHRMS (m/z) 277.1048 (M+H, C₁₅H₁₆O₅ requires 277.1071).

Step 5: Preparation of Methyl 7-methoxy-4-methyl-4,5-dihydro-2H-benzo[g]indazole-3-carboxylate.

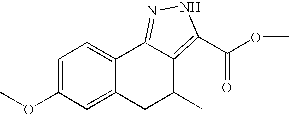

Methyl (2Z)-hydroxy(6-methoxy-3-methyl-1-oxo-3,4-dihydronaphthalen-2(1H)-ylidene)ethanoate (2.00 g, 7.34 mmol) was dissolved in methanol (50 mL). Hydrazine hydrochloride (0.553 g, 8.08 mmol was added. Stirred at reflux for two hours. Cooled to room temperature and neutralized with NaHCO₃ (aq.). Water was added, resulting in formation of a precipitate. Filtered and washed with H₂O. (1.84 g, 92%) mp 162.7-162.8° C.: ¹H NMR (CDCl₃/300 MHz) 7.73 (d, 1H), 6.84 (d, 1H), 6.82 (s, 1H), 3.95 (s, 3H), 3.83 (s, 3H), 3.53 (t, 1H), 3.20 (dd, 1H), 2.73-2.65 (m, 1H), 1.10 (d, 3H). ESHRMS (m/z) 273.1216 (M+H, C₁₅H₁₆N₂O₃ requires 273.1234).

Step 6: Preparation of 2-(3-Aminopropyl)-7-hydroxy-4-methyl-2H-benzo[g]indazole-3-carboxylic Acid Trifluoroacetate.

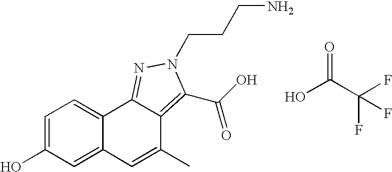

Methyl 7-methoxy-4-methyl-4,5-dihydro-2H-benzo[g]indazole-3-carboxylate (2.02 g, 7.42 mmol) was dissolved in CH₂Cl₂ (100 mL). A mixture of KMnO₄/CuSO₄.5H₂O (1:1 by weight, 15.05 g, 37 mmol) was added. The reaction mixture was heated at reflux for 5 days. An additional 1 g of KMnO₄/CuSO₄.5H₂O was added each day. The heating mantle was removed and ethanol (100 mL) was added to the reaction mixture. Stirred at room temperature for 30 minutes, then filtered through a plug of silica. The solvent was evaporated to yield a yellow solid. 15% of non-oxidized product present by analysis. (0.128 g, 6%). This mixture (0.128 g, 0.474 mmol) was dissolved in DMF (5 mL). Cooled to 0° C. in an ice-water bath. Lithium-t-butoxide (0.6 mL, 0.6 mmol of a 1.0 M solution in THF) was added via syringe. Stirred for one hour. Boc-3-bromopropylamine (0.0.135 g, 0.567 mmol) in DMF (5 mL) was added. The ice-bath was removed and the reaction stirred at room temperature for four hours. Quenched with NH4Cl (aq.) (20 mL), and the mixture was extracted with ethyl acetate (2×50 mL). The combined organic phases were washed with brine, dried over MgSO₄, filtered, and evaporated to yield a orange solid, which was taken on with no further purification or isolation.

The ester (0.203 g, 0.474 mmol) was dissolved in a THF/ethanol/water mixture (10 mL, 7/2/1 respectively). Lithium hydroxide monohydrate (0.040 g, 0.948 mmol was added. Stirred overnight at room temperature. Diluted with ethyl acetate (50 mL) and extracted with 0.1N HCl (2×25 mL), and brine (25 mL). The organic phase was dried over MgSO₄, filtered, and evaporated. The orange solid was taken to the next step with no further purfication or isolation.

The carboxylic acid (0.196 g, 0.474 mmol) was slurried in CH₂Cl₂ (10 mL). The mixture was cooled to −20° C. Boron tribromide (3 mL, 3 mmol of a 1.0 M solution in CH₂Cl₂) was added dropwise via syringe. The mixture stirred at room temperature for three hours. H₂O was slowly added dropwise to quench the reaction. The solvent was evaporated and the resulting solid was purified by preparative reverse phase HPLC to yield a white solid (0.060 g): ¹H NMR (DMSO tfa/300 MHz) 8.21 (d, 1H), 7.72 (s, 2H), 7.17 (s, 1H), 7.09-7.03 (m, 2H), 4.69 (t, 2H), 2.86 (t, 2H), 2.56 (s, 3H) 2.20(qt, 2H). ESHRMS (m/z) 300.1321 (M+H, C₁₆H₁₇N₃O₃ requires 300.1343).

### EXAMPLE 83

This example illustrates the production of 2-(3-aminopropyl)-7-hydroxy-4-methyl-4,5-dihydro-2H-benzo[g]indazole-3-carboxylic acid trifluoroacetate.

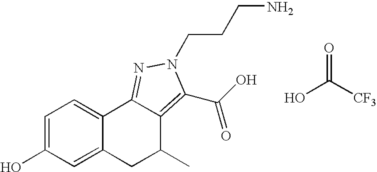

Step 1: Preparation of 2-{3-[(tert-butoxycarbonyl)amino]propyl}-7-methoxy-4-methyl-4,5-dihydro-2H-benzo[g]indazole-3-carboxylic Acid.

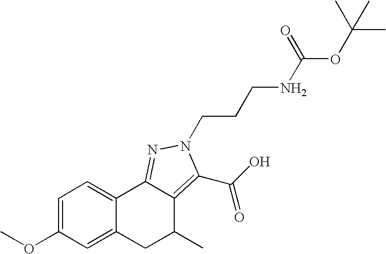

Methyl 7-methoxy-4-methyl-4,5-dihydro-2H-benzo[g]indazole-3-carboxylate (0.500 g, 1.84 mmol) was dissolved in DMF (5 mL). Cooled to 0° C. in an ice-water bath. Lithium-t-butoxide (2.2 mL, 2.2 mmol of a 1.0 M solution in THF) was added via syringe. Stirred for one hour. Boc-3-bromopropylamine (0.524 g, 2.2 mmol) in DMF (5 mL) was added. The ice-bath was removed and the reaction stirred at room temperature for four hours. Quenched with NH₄Cl (aq.) (20 mL), and the mixture was extracted with ethyl acetate (2×50 mL). The combined organic phases were washed with brine, dried over MgSO₄, filtered, and evaporated. Purified by flash column chromatography in 20% ethyl acetate/80% hexane. Obtained a slightly orange oil (0.53 g, 67%). The ester (0.517 g, 1.2 mmol) was dissolved in a THF/ethanol/water mixture (10 mL, 7/2/1 respectively). Lithium hydroxide monohydrate (0.100 g, 2.40 mmol was added. Stirred overnight at room temperature. Diluted with ethyl acetate (50 mL) and extracted with 0.1N HCl (2×25 mL), and brine (25 mL). The organic phase was dried over MgSO₄, filtered, and evaporated. Recrystallized from ethyl acetate/hexane (0.345 g, 45%): ¹H NMR (DMSO tfa/300 MHz) 7.61 (d, 1H), 6.86 (s, 1H), 6.84 (d, 2H), 4.43 (t, 2H), 3.76 (s, 3H), 3.44 (t, 1H), 3.09-2.92 (m, 3H), 2.72-2.65 (m, 1H), 1.87 (qt, 2H), 1.36 (s, 9H), 0.96 (d, 3H). ESHRMS m/z 416.2197 (M+H, C₂₂H₂₉N₃O₅ requires 416.2180).

Step 2: Preparation of 2-(3-aminopropyl)-7-hydroxy-4-methyl-4,5-dihydro-2H-benzo[g]indazole-3-carboxylic Acid Trifluoroacetate.

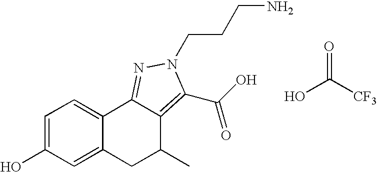

2-{3-[(tert-butoxycarbonyl)amino]propyl}-7-methoxy-4-methyl-4,5-dihydro-2H-benzo[g]indazole-3-carboxylic acid (0.200 g, 0.480 mmol) was slurried in CH₂Cl₂ (10 mL). The mixture was cooled to −20° C. Boron tribromide (5 mL, 5 mmol of a 1.0 M solution in CH₂Cl₂) was added dropwise via syringe. The mixture stirred at room temperature for three hours. Slowly added H₂O dropwise to quench the reaction. The solvent was evaporated and the resulting solid was purified by preparative reverse phase HPLC to yield a white solid (0.079 g): ¹H NMR (DMSO tfa/300 MHz) 7.72 (s, 2H), 7.50 (d, 1H), 6.68 (s, 1H), 6.65 (d, 1H), 4.52 (t, 2H), 3.42 (t, 1H), 3.04-2.97 (m, 1H), 2.85-2.75 (m, 2H), 2.64-2.49 (m, 1H), 2.05 (qt, 2H), 0.96 (d, 3H). ESHRMS (m/z) 302.1527 (M+H, C₁₆H₁₉N₃O₃ requires 302.1499).

### EXAMPLE 84

This example illustrates the production of 2-(3-Aminopropyl)-7-hydroxy-5-methyl-2H-benzo[g]indazole-3-carboxylic acid trifluoroacetate.

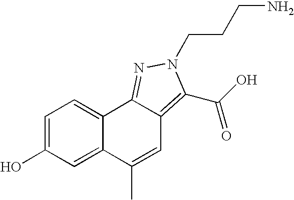

Step 1: Preparation of 6-methoxy-4-methyl-3,4-dihydronaphthalen-1 (2H)-one.

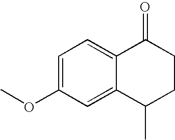

A 2L three-necked round-bottomed flask was equipped with and overhead stirred and two rubber septa. The flask was cooled to 0° C., and charged with anhydrous Cerium (III) chloride 71 g, 288 mmol) and anhydrous THF (500 mL). Stirred overnight at room temperature. The flask was cooled to −78° C. Methyllithium (229 mL, 320 mmol of a 1.4 M solution in ether) was added via cannula. Stirred at −78° C. for 30 minutes, and 7-methoxy tetralone (50.0 g, 288 mmol) in THF (200 mL) was added via addition funnel. The cooling bath was removed and the reaction stirred at room temperature for three hours. Quenched with 5% acetic acid (300 mL). Extracted with ether (2×300 mL). The combined organic phases were washed with brine, dried over MgSO₄, filtered, and evaporated. Obtained a pale orange oil, which contained a mixture of both the tertiary alcohol, and the alkene. (54 g). The mixture (52.23 g) was dissolved in dichloromethane (100 mL). Triethylsilane (63 g, 543 mmol) was added, and the solution was cooled to 0° C. Trifluoroacetic acid (208 mL) was slowly added via addition funnel. The ice bath was removed and the reaction stirred at room temperature overnight. Dichloromethane and trifluoroacetic acid were evaporated. Sodium bicarbonate (aq.) was added to consume any remaining trifluoroacetic acid. Extracted with dichloromethane (2×200 mL). The combined organic phases were washed with brine, dried over MgSO₄, filtered, and evaporated. Purified by flash column chromatography in hexane. Obtained desired product with non-methylated impurity (20%). (37.43 g).

The mixture (8.31 g) was dissolved in acetic acid (60 mL). Chromium trioxide (9.43 g, 94.3 mmol) was slowly added in small portions over two hours. The reaction mixture was heated to 90° C. for four hours. Stirred at room temperature overnight and evaporated the acetic acid. The remaining residue was neutralized with sodium bicarbonate (aq.) and then extracted with ethyl acetate (2×200 mL). The combined organic phases were washed with brine, dried over MgSO₄, filtered, and evaporated. Purified by flash column chromatography in 5% ethyl acetate/95% hexane. The resulting solid was recrystallized from hexane (3.96 g 44%): mp 57.5-58.5° C. ¹H NMR (CDCl₃/300 MHz) 8.02 (d, 1H), 6.82 (d, 1H), 6.78 (s, 1H), 3.87 (s, 3H), 3.08-3.02 (m, 1H), 2.77-2.52 (m, 2H), 2.26-2.19 (m, 1H), 1.92-1.86 (m, 1H), 1.39 d, 3H). ESHRMS (m/z) 191.1092 (M+H, C₁₂H₁₄O₂ requires 191.1067).

Step 2: Preparation of Methyl 7-methoxy-5-methyl-4,5-dihydro-2H-benzo[g]indazole-3-carboxylate,

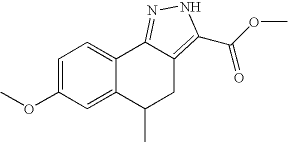

6-Methoxy-4-methyl-3,4-dihydronaphthalen-1 (2H)-one (3.00 g, 15.8 mmol) was dissolved in ether (50 mL). Dimethyl oxalate (2.05 g, 17.4 mmol) and sodium methoxide (3.98 mL, 3.76 g, 17.4 mmol of a 25%/wt. solution in methanol) were added. Stirred at room temperature overnight. Added 1 N HCl to pH=1. The solution was transferred to a separatory funnel and the layers were separated. The aqueous phase was washed with ethyl acetate (2×100 mL). The combined organic phases were washed with brine (200 mL), dried over MgSO₄, filtered, and evaporated. The resulting oil was filtered through a plug of silica with 20% ethyl acetate/80% hexane. Obtained an orange oil (3.48 g, 80%)

The oil (3.48 g, 12.6 mmol) was dissolved in methanol (50 mL). Hydrazine hydrochloride (0.95 g, 13.8 mmol was added. Stirred at reflux for two hours. Cooled to room temperature and neutralized with NaHCO₃ (aq.). Water was added, resulting in formation of a precipitate. Filtered and washed with H₂O. (2.23 g, 65%): ¹H NMR (CDCl₃/300 MHz) 7.76 (d, 1H), 6.86 (s, 1H), 6.82 (d, 1H), 3.95 (s, 3H), 3.85 (s, 3H), 3.14-3.01 (m, 2H), 2.97-2.84 (m, 2H), 1.25 (d, 3H). ESHRMS (m/z) 273.1215 (M+H, C₁₅H₁₆N₂O₃ requires 273.1234).

Step 3: Preparation of Methyl 7-methoxy-5-methyl-2H-benzo[g]indazole-3-carboxylate.

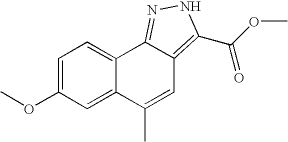

Methyl 7-methoxy-5-methyl-4,5-dihydro-2H-benzo[g]indazole-3-carboxylate (2.58 g, 9.49 mmol) was dissolved in CH₂Cl₂ (100 mL). A mixture of KMnO₄/CuSO₄.5H₂O (1:1 by weight, 7.73 g, 19 mmol was added the reaction mixture was heated at reflux for 5 days. An additional 1 g of KMnO₄/CuSO₄.5H₂O was added each day. The heating mantle was removed and ethanol (100 mL) was added to the reaction mixture. Stirred at room temperature for 30 minutes, then filtered through a plug of silica. The solvent was evaporated to yield a yellow solid. Trituration with CH₂Cl₂ removed any remaining starting material. (0.503 g, 20%): ¹H NMR (CDCl₃/300 MHz) 8.46 (d, 1H), 7.91 (s, 1H), 7.47-7.40 (m, 2H), 3.97 (s, 3H), 3.96 (s, 3H), 2.69 (s, 3H). ESHRMS (m/z) 271.1082 (M+H, C₁₅H₁₄N₂O₃ requires 271.1077).

Step 4: Preparation of Methyl 2-{3-[(tert-butoxy-carbonyl)amino]propyl}-7-methoxy-5-methyl-2H-benzo[g]indazole-3-carboxylate.

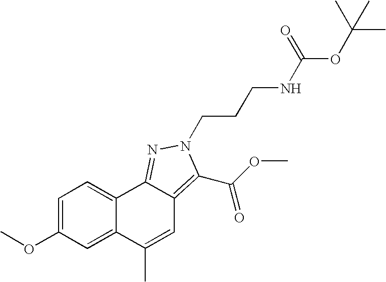

Methyl 7-methoxy-5-methyl-2H-benzo[g]indazole-3-carboxylate (0.401 g, 1.48 mmol) was dissolved in DMF (10 mL). Cooled to 0° C. in an ice-water bath. Lithium-t-butoxide (3 mL, 3 mmol of a 1.0 M solution in THF) was added via syringe. Stirred for one hour. Boc-3-bromopropylamine (0.529 g, 2.22 mmol) in DMF (5 mL) was added. The ice-bath was removed and the reaction stirred at room temperature for four hours. Quenched with NH₄Cl (aq.) (20 mL), and the mixture was extracted with ethyl acetate (2×50 mL). The combined organic phases were washed with brine, dried over MgSO₄, filtered, and evaporated to yield a brown oil. Purified by flash column chromatography in 15% ethyl acetate/85% hexane. The resulting solid was recrystallized from ethanol/water. (0.350 g, 55%): ¹H NMR (CDCl₃/300 MHz) 8.55 (d, 1H), 7.73 (s, 1H), 7.35 (d, 1H), 7.28 (m, 1H), 5.14 (s, 1H), 4.94 (t, 2H), 4.06 (s, 3H), 3.97 (s, 3H), 3.12 (t, 2H), 2.66 (s, 3H), 2.16 (qt, 2H). ESHRMS (m/z) 428.2210 (M+H, C₂₃H₂₉N₃O₅ requires 428.2180).

Step 5: Preparation of 2-{3-[(tert-butoxy-carbonyl)-amino]propyl}-7-methoxy-5-methyl-2H-benzo[g]indazole-3-carboxylic Acid.

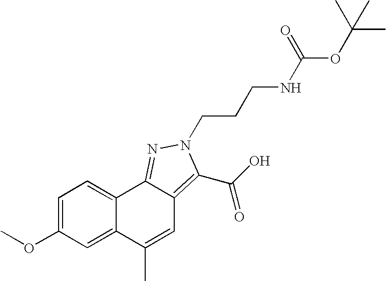

Methyl 2-{3-[(tert-butoxy-carbonyl)amino]propyl}-7-methoxy-5-methyl-2H-benzo[g]indazole-3-carboxylate (0.320 g, 0.748 mmol was dissolved in a THF/ethanol/water mixture (10 mL, 7/2/1 respectively). Lithium hydroxide monohydrate (0.079 g, 1.9 mmol was added. Stirred overnight at room temperature. Diluted with ethyl acetate (50 mL) and extracted with 0.1N HCl (2×25 mL), and brine (25 mL). The organic phase was dried over MgSO₄, filtered, and evaporated. The resulting white solid was recrystallized from ethyl acetate/hexane. (0.220 g, 71%): ¹H NMR (DMSO tfa/300 MHz) 8.44 (d, 1H), 7.71 (d, 1H), 7.41 (d, 1H), 7.31 (dd, 1H), 6.95 (s, 1H), 4.84 (t, 2H), 3.94 (s, 3H), 3.00 (t, 2H), 2.63 (s, 3H), 2.04 (qt, 2H). ESHRMS (m/z) 414.2025 (M+H, C₂₃H₂₉N₃O₅ requires 414.2023).

Step 6: 2-(3-aminopropyl)-7-hydroxy-5-methyl-2H-benzo[g]indazole-3-carboxylic Acid Trifluoroacetate.

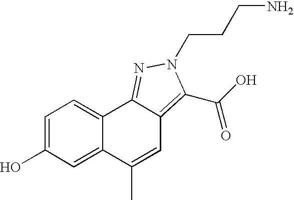

2-{3-[(tert-butoxy-carbonyl)amino]propyl}-7-methoxy-5-methyl-2H-benzo[g]indazole-3-carboxylic acid (0.175 g, 0.424 mmol) was slurried in CH₂Cl₂ (10 mL). The mixture was cooled to −20° C. Boron tribromide (4.5 mL, 4.5 mmol of a 1.0 M solution in CH₂Cl₂) was added dropwise via syringe. The mixture stirred at room temperature for three hours. Slowly added H₂O dropwise to quench the reaction. The solvent was evaporated and the resulting solid was purified by preparative reverse phase HPLC to yield a white solid (0.060 g): ¹H NMR (DMSO tfa/300 MHz) 8.35 (d, 1H), 7.73 (d, 3H), 7.31 (d, 1H), 7.18 (dd, 1H), 4.90 (t, 2H), 2.86 (t, 2H), 2.57 (s, 3H), 2.21 (qt, 2H). ESHRMS (m/z) 300.1347 (M+H, C₁₆H₁₇N₃O₃ requires 300.1343).

### EXAMPLE 85

This example illustrates the production of 2-(3-aminopropyl)-7-hydroxy-5-methyl-4,5-dihydro-2H-benzo[g]indazole-3-carboxylic acid trifluoroacetate.

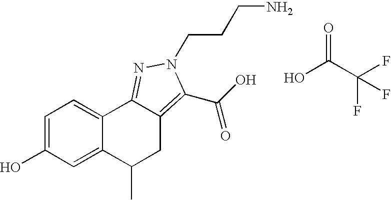

Step 1: Preparation of 2-{3-[(tert-butoxycarbonyl)amino]propyl}-7-methoxy-5-methyl-4,5-dihydro-2H-benzo[g]indazole-3-carboxylic Acid.

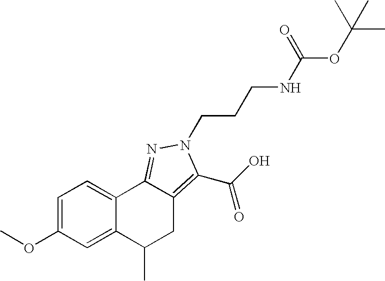

Methyl 7-methoxy-5-methyl-4,5-dihydro-2H-benzo[g]indazole-3-carboxylate (1.00 g, 3.67 mmol) was dissolved in DMF (15 mL). Cooled to ⁰° C. in an ice-water bath. Lithium-t-butoxide (5.5 mL, 5.5 mmol of a 1.0 M solution in THF) was added via syringe. Stirred for one hour. Boc-3-bromopropylamine (1.31 g, 5.5 mmol) in DMF (15 mL) was added. The ice-bath was removed and the reaction stirred at room temperature for four hours. Quenched with NH₄Cl (aq.) (50 mL), and the mixture was extracted with ethyl acetate (2×100 mL). The combined organic phases were washed with brine, dried over MgSO₄, filtered, and evaporated. Purified by flash column chromatography in 15% ethyl acetate/85% hexane. Obtained a slightly orange oil. The ester (1.00 g, 2.33 mmol) was dissolved in a THF/ethanol/water mixture (10 mL, 7/2/1 respectively). Lithium hydroxide monohydrate (0.196 g, 4.66 mmol was added. Stirred overnight at room temperature. Diluted with ethyl acetate (50 mL) and extracted with 0.1N HCl (2×25 mL), and brine (25 mL). The organic phase was dried over MgSO₄, filtered, and evaporated. Recrystallized from ethanol/water (0.734 g, 48%): ¹H NMR (DMSO tfa/300 MHz) 7.65 (d, 1H), 6.90 (s, 1H), 6.87 (d, 1H), 4.49 (t, 2H), 3.79 (s, 3H), 2.99-2.79 (m, 5H), 1.93 (qt, 2H), 1.39 (s, 9H), 1.17 d, 3H). ESHRMS m/z 416.2179 (M+H, C₂₂H₂₉N₃O₅ requires 416.2180).

Step 2: Preparation of 2-(3-aminopropyl)-7-hydroxy-5-methyl-4,5-dihydro-2H-benzo[g]indazole-3-carboxylic Acid Trifluoroacetate.

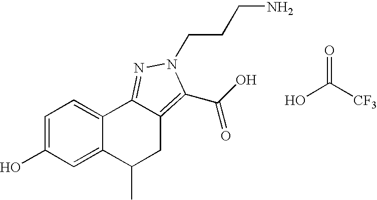

2-{3-[(tert-butoxycarbonyl)amino]propyl}-7-methoxy-5-methyl-4,5-dihydro-2H-benzo[g]indazole-3-carboxylic acid (0.300 g, 0.722 mmol) was slurried in CH₂Cl₂ (10 mL). The mixture was cooled to −20° C. Boron tribromide (7 mL, 7 mmol of a 1.0 M solution in CH₂Cl₂) was added dropwise via syringe. The mixture stirred at room temperature for three hours. Slowly added H₂O dropwise to quench the reaction. The solvent was evaporated and the resulting solid was purified by preparative reverse phase HPLC to yield a white solid (0.100 g): ¹H NMR (DMSO tfa/300 MHz) 7.71 (s, 2H), 7.55 (d, 1H), 6.74 (s, 1H), 6.70 (d, 1H), 4.58 (t, 2H), 3.02-2.85 (m, 5H), 2.08 (qt, 2H), 1.15 (d, 3H). ESHRMS (m/z) 302.1532 (M+H, C₁₆H₁₉N₃O₃ requires 302.1499).

### EXAMPLE 85

This example illustrates the production of 7-hydroxy-5-methyl-4,5-dihydro-2H-benzo[g]indazole-3-carboxylic acid.

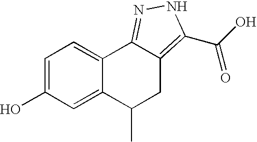

Step 1: Preparation of Methyl 7-hydroxy-5-methyl-4,5-dihydro-2H-benzo[g]indazole-3-carboxylate.

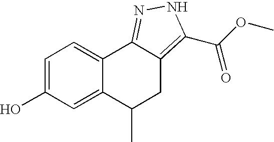

Methyl 7-methoxy-5-methyl-4,5-dihydro-2H-benzo[g]indazole-3-carboxylate (0.400 g, 1.47 mmol) was dissolved in CH₂Cl₂ (10 mL). The mixture was cooled to −20° C. Boron tribromide (7.5 mL, 7.5 mmol of a 1.0 M solution in CH₂Cl₂) was added dropwise via syringe. The mixture stirred at room temperature for three hours. Slowly added methanol dropwise to quench the reaction. The solvent was evaporated. Purified by flash column chromatography in 15:1 CH₂Cl₂:CH₃OH to yield a brown solid which was recrystallized from ethanol/water (0.100 g, 26%): ¹H NMR (DMSO tfa/300 MHz)7.59 (d, 1H), 6.79 (s, 1H), 6.75 (d, 1H), 3.83 (s, 3H), 3.07-2.98 (m, 2H), 2.90-2.75 (m, 1H), 1.16 (d, 3H). ESHRMS (m/z) 259.1051 (M+H, C₁₄H₁₄N₂O₃ requires 259.1077).

Step 2: Preparation of 7-hydroxy-5-methyl-4,5-dihydro-2H-benzo[g]indazole-3-carboxylic Acid.

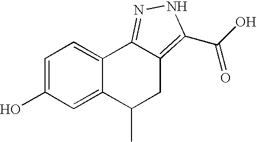

Methyl 7-hydroxy-5-methyl-4,5-dihydro-2H-benzo[g]indazole-3-carboxylate (0.050 g, 0.184 mmol) was dissolved in a THF/ethanol/water mixture (10 mL, 7/2/1 respectively). Lithium hydroxide monohydrate (0.025 g 0.60 mmol) was added. Stirred overnight at room temperature. THF was evaporated. 1N HCl was added until solution turned clouldy. The resulting white precipitate was filtered. (0.037 g, 82%): ¹H NMR (DMSO tfa/300 MHz) 7.71 (d, 1H), 6.88 (s, 1H), 6.82 (d, 1H), 3.83 (s, 3H), 3.13-2.95 (m, 3H), 1.26 (d, 3H). ESHRMS m/z 245.0917 (M+H, C₁₃H₁₂N₂O₃ requires 245.0921).

### EXAMPLE 86

This example illustrates the production of 8-bromo-7-hydroxy-4,5-dihydro-2H-benzo[g]indazole-3-carboxylic acid,

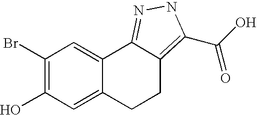

Step 1: Preparation of ethyl 8-bromo-7-methoxy-4,5-dihydro-2H-benzo[g]indazole-3-carboxylate.

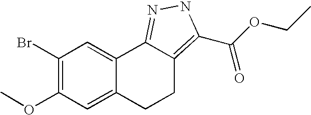

Ethyl 7-methoxy-4,5-dihydro-2H-benzo[g]indazole-3-carboxylate (1.0 g, 3.67 mmol) was dissolved in acetic acid (20 mL). Sodium acetate (0.301 g, 3.67 mmol) was added. Cooled to 15° C. in an ice-water bath, and added bromine (0.586 g, 3.67 mmol) in acetic acid (10 mL). The ice bath was removed, and the reaction stirred at room temperature for three hours. The acetic acid was evaporated, and the resulting solid was dissolved in ethyl acetate (100 mL). Extracted with NaHCO₃ (aq.) (2×50 mL) and brine (50 mL). The organic phase was dried over MgSO₄, filtered, and evaporated. The resulting solid was recrystallized from ethyl acetate/hexane to afford a white solid. (0.5 g, 39%): ¹H NMR (CDCl₃/300 MHz 8.03 (s, 1H), 6.80 (s, 1H), 4.41 (q, 2H), 3.92 (s, 3H), 3.07-2.91 (m, 4H), 1.42 (t, 3H). ESHRMS m/z 353.0321 (M+H, C₁₅H₁₅BrN₂O₃ requires 353.0320).

Step 2: Preparation of 8-bromo-7-hydroxy-4,5-dihydro-2H-benzo[g]indazole-3-carboxylic Acid.

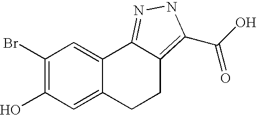

Ethyl 8-bromo-7-methoxy-4,5-dihydro-2H-benzo[g]indazole-3-carboxylate (0.357 g, 1.02 mmol) was dissolved in CH₂Cl₂ (20 mL). The mixture was cooled to −20° C. Boron tribromide (5 mL, 5 mmol of a 1.0 M solution in CH₂Cl₂) was added dropwise via syringe. The mixture stirred at room temperature for three hours. Slowly added ethanol dropwise to quench the reaction. The solvent was evaporated and the resulting solid was washed with water and filtered. The solid was a mixture of ester and carboxylic acid. It was dissolved in a THF/ethanol/water mixture (10 mL, 7/2/1 respectively). Lithium hydroxide monohydrate (0.086 g, 2.04 mmol) was added. Stirred overnight at room temperature. 1N HCl was added to form a white precipitate, which was filtered. (0.250 g, 79%): ¹H NMR (DMSO/300 MHz 7.77 (s, 1H), 6.93 (s, 1H), 2.91-2.80 (m, 4H). ESHRMS m/z 310.9875 (M+H, C₁₂H₉BrN₂O₃ requires 310.9850).

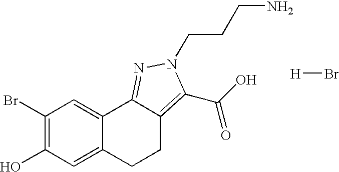

2-(3-aminopropyl)-8-bromo-7-hydroxy-4,5-dihydro-2H-benzo[g]indazole-3-carboxylic acid hydrobromide

Step 1: Preparation of ethyl 8-bromo-2-{3-[(tert-butoxycarbonyl)amino]propyl}-7-methoxy-4,5-dihydro-2H-benzo[g]indazole-3-carboxylate.

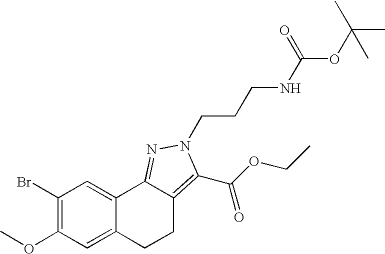

Ethyl 8-bromo-7-methoxy-4,5-dihydro-2H-benzo[g]indazole-3-carboxylate (1.76 g, 5 mmol) was dissolved in DMF (20 mL). Cooled to 0° C., in an ice-water bath. Lithium t-butoxide (11 mL, 11 mmol of a 1.0 M solution in THF) was added dropwise via syringe. Stirred for 2 hours. N-boc-3-bromopropylamine (2.63 g, 11 mmol) in DMF (20 mL) was added via syringe. The ice bath was removed and the reaction stirred at room temperature overnight. Quenched with NH₄Cl (aq.) (50 mL), and the layers were separated. The aqueous phase was extracted with ethyl acetate (2×100 mL). The combined organic phases were washed with brine, (100 mL), dried over MgSO₄, filtered, and evaporated. The resulting brown oil solidified upon standing. Recrystallized from ethanol/water to afford a grey solid (1.48 g, 58%): ¹H NMR (CDCl₃/300 MHz 8.00 (s, 1H), 6.78 (s, 1H), 4.62 (t, 2H) 4.39 (q, 2H), 3.92 (s, 3H), 3.12-2.91 (m, 6H), 2.05 (t, 2H), 1.44 (s, 9H), 1.42 (t, 3H). ESHRMS m/z 508.1469 (M+H, C₂₃H₃₀BrN₃O₅ requires 508.1442).

Step 2: Preparation of 8-bromo-2-{3-[(tert-butoxycarbonyl)amino]propyl}-7-methoxy-4,5-dihydro-2H-benzo[g]indazole-3-carboxylic Acid,

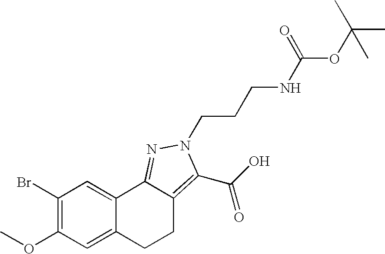

Ethyl 8-bromo-2-{3-[(tert-butoxycarbonyl)amino]propyl}-7-methoxy-4,5-dihydro-2H-benzo[g]indazole-3-carboxylate (0.508 g, 1.00 mmol) was dissolved in a THF/ethanol/water mixture (10 mL, 7/2/1 respectively). Lithium hydroxide monohydrate (0.105 g, 2.5 mmol) was added. Stirred overnight at room temperature. Diluted with ethyl acetate (50 mL) and extracted with 0.1N HCl (2×25 mL), and brine (25 mL). The organic phase was dried over MgSO₄, filtered, and evaporated. The resulting white solid was recrystallized from ethyl acetate/hexane. (0.433 g, 99%): ¹H NMR (DMSO/300 MHz 7.73 (s, 1H), 7.07 (s, 1H), 4.61 (t, 2H), 3.88(s, 3H), 2.95-2.81 (m, 6H), 1.85 (t, 2H), 1.4 (t, 9H). ESHRMS m/z 480.1145 (M+H, C₂₁H₂₆BrN₃O₅ requires 480.1129).

Step 3: Preparation of 2-(3-aminopropyl)-8-bromo-7-hydroxy-4,5-dihydro-2H-benzo[g]indazole-3-carboxylic Acid Hydrobromide.

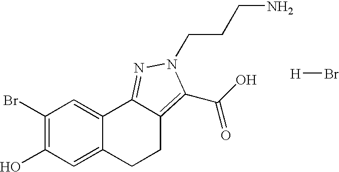

8-bromo-2-{3-[(tert-butoxycarbonyl)amino]propyl}-7-methoxy-4,5-dihydro-2H-benzo[g]indazole-3-carboxylic acid (0.254 g, 0.529 mmol) was slurried in CH₂Cl₂ (5 mL). The mixture was cooled to −20° C. Boron tribromide (2.5 mL, 2.5 mmol of a 1.0 M solution in CH₂Cl₂) was added dropwise via syringe. The mixture stirred at room temperature for three hours. Slowly added H₂O dropwise to quench the reaction. The solvent was evaporated and the resulting solid was triturated with acetonitrile (0.190 g): ¹H NMR (DMSO/300 MHz) 7.75 (s, 1H), 6.91 (s, 1H), 4.59 (t, 2H), 2.94-2.83 (m, 6H), 2.10 (t, 2H). ESHRMS m/z 366.0430 (M+H, C₁₅H₁₆BrN₃O₃ requires 366.0448).

### EXAMPLE 87

This example illustrates the production of 2-(3-aminopropyl)-8-bromo-7-hydroxy-2H-benzo[g]indazole-3-carboxylic acid trifluoroacetate.

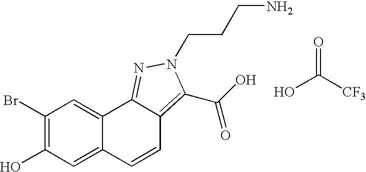

Step 1: Preparation of ethyl 8-bromo-7-methoxy-2H-benzo[g]indazole-3-carboxylate

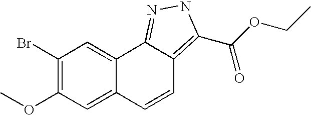

Ethyl 8-bromo-7-methoxy-4,5-dihydro-2H-benzo[g]indazole-3-carboxylate (0.351 g, 1.00 mmol) was dissolved in anhydrous dioxane (20 mL). DDQ (0.227 g, 1.00 mmol) was added. Stirred at reflux for 10 hours. Stirred at room temperature overnight. The resulting solid was filtered, and washed with ethyl acetate (50 mL). The mother liquor was extracted with 1.0 N NaOH until the yellow color dissipated. Checked combined aqueous extracts by TLC for any product. Back extracted with ethyl acetate until the entire product was in organic extracts. The combined organic phases were washed with brine, dried over MgSO₄, filtered and evaporated to yield an orange solid. Recrystallized from ethyl acetate/hexane (0.150 g, 43%). ¹H NMR (DMSO/300 MHz) 8.83 (s, 1H) 8.07 (d, 1H), 7.74 (s, 1H), 7.72 (d, 1H), 4.44 (q, 2H), 4.04 (s, 3H) 1.43 (t, 2H). ESHRMS m/z 349.0182 (M+H, C₁₅H₁₃BrN₂O₃ requires 349.0182).

Step 2: Preparation of ethyl 8-bromo-2-{3-[(tert-butoxycarbonyl)amino]propyl}-7-methoxy-2H-benzo[g]indazole-3-carboxylate.

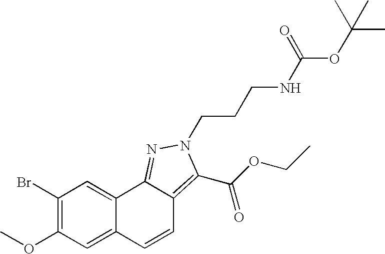

Ethyl 8-bromo-7-methoxy-2H-benzo[g]indazole-3-carboxylate (0.650 g, 1.86 mmol) was dissolved in DMF (20 mL). Cooled to 0° C., in an ice-water bath. Lithium t-butoxide (3.72 mL, 3.72 mmol of a 1.0 M solution in THF) was added dropwise via syringe. Stirred for 2 hours. N-boc-3-bromopropylamine (0.885 g, 3.72 mmol) in DMF (5 mL) was added via syringe. The ice bath was removed and the reaction stirred at room temperature overnight. Quenched with NH₄Cl (aq.) (50 mL), and the layers were separated. The aqueous phase was extracted with ethyl acetate (2×50 mL). The combined organic phases were washed with brine, (50 mL), dried over MgSO₄, filtered, and evaporated. The resulting yellow oil was purified by flash column chromatography in 20% ethyl acetate/80% hexane. (0.690 g, 73%): ¹H NMR (CDCl₃/300 MHz) 8.79 (s, 1H), 7.92 (d, 1H), 7.47 (d, 1H), 7.24 (s, 1H), 5.05 (s, 1H), 4.96 (t, 2H), 4.51 (q, 2H), 4.04 (s, 3H), 3.14 (q, 2H), 2.18 (qt, 2H), 1.53 (t, 3H), 1.44 (s, 9H). ESHRMS m/z 506.1259 (M+H, C₂₃H₂₈BrN₃O₅ requires 506.1296).

Step 3: Preparation of 8-bromo-2-{3-[(tert-butoxy-carbonyl)amino]propyl}-7-methoxy-2H-benzo[g]indazole-3-carboxylic Acid.

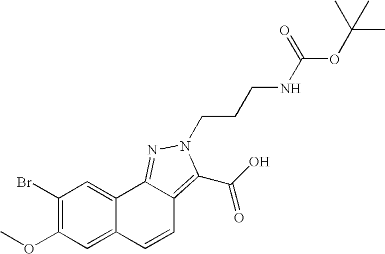

Ethyl 8-bromo-2-{3-[(tert-butoxycarbonyl)amino]propyl}-7-methoxy-2H-benzo[g]indazole-3-carboxylate (0.506 g, 1.0 mmol) was dissolved in a THF/ethanol/water mixture (10 mL, 7/2/1 respectively). Lithium hydroxide monohydrate (0.105 g, 2.50 mmol) was added. Stirred overnight at room temperature. Diluted with ethyl acetate (50 mL) and extracted with 0.1N HCl (2×25 mL), and brine (25 mL). The organic phase was dried over MgSO₄, filtered, and evaporated. The resulting white solid was recrystallized from ethyl acetate/hexane. (0.414 g, 87%): ¹H NMR (DMSO/300 MHz) 8.55 (s, 1H), 8.13 (d, 1H), 7.59 (s, 1H), 7.47 (d, 1H), 7.05 (s, 1H), 4.94 (t, 2H), 4.00 (s, 3H), 2.96 (q, 2H), 2.02 (qt, 2H), 1.39 (s, 9H). ESHRMS m/z 478.0988 (M+H, C₂₁H₂₄BrN₃O₅ requires 478.0983).

Step 4: Preparation of 2-(3-aminopropyl)-8-bromo-7-hydroxy-2H-benzo[g]indazole-3-carboxylic Acid Trifluoroacetate.

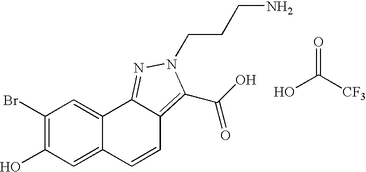

8-bromo-2-{3-[(tert-butoxy-carbonyl)amino]propyl}-7-methoxy-2H-benzo[g]indazole-3-carboxylic acid (0.310 g, 0.648 mmol) was slurried in CH₂Cl₂ (10 mL). The mixture was cooled to −20° C. Boron tribromide (4 mL, 4 mmol of a 1.0 M solution in CH₂Cl₂) was added dropwise via syringe. The mixture stirred at room temperature for three hours. Slowly added H₂O dropwise to quench the reaction. The solvent was evaporated and the resulting solid was purified by preparative reverse phase HPLC to obtain a white solid (0.170 g): ¹H NMR (DMSO tfa/300 MHz) 8.56 (s, 1H), 7.88 (d, 1H), 7.44 (s, 2H), 7.53 (d, 1H), 7.44 (s, 1H), 4.93 (t, 2H), 2.87 (q, 2H), 2.24 (qt, 2H). ESHRMS m/z 366.0275 (M+H, C₁₅H₁₄BrN₃O₃ requires 366.0291).

### EXAMPLE 88

This example illustrates the production of 2-(2-aminoethyl)-8-bromo-7-hydroxy-4,5-dihydro-2H-benzo[g]indazole-3-carboxylic acid trifluoroacetate.

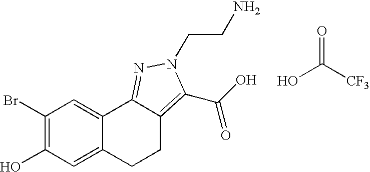

Step 1: Preparation of ethyl 8-bromo-2-{2-[(tert-butoxycarbonyl)amino]ethyl}-7-methoxy-4,5-dihydro-2H-benzo[g]indazole-3-carboxylate.

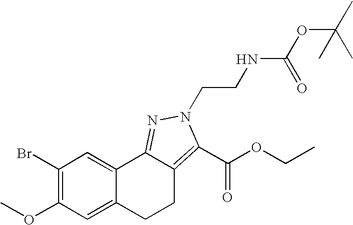

Ethyl 8-bromo-7-methoxy-4,5-dihydro-2H-benzo[g]indazole-3-carboxylate (0.710 g, 2.02 mmol) was dissolved in DMF (10 mL). Cooled to 0° C., in an ice-water bath. Lithium t-butoxide (4.45 mL, 4.45 mmol of a 1.0 M solution in THF) was added dropwise via syringe. Stirred for 2 hours. N-boc-2-bromoethylamine (1.08 g, 4.45 mmol) in DMF (10 mL) was added via syringe. The ice bath was removed and the reaction stirred at room temperature overnight. Quenched with NH₄Cl (aq.) (50 mL), and the layers were separated. The aqueous phase was extracted with ethyl acetate (2×100 mL). The combined organic phases were washed with brine, (100 mL), dried over MgSO₄, filtered, and evaporated to afford a white solid. Recrystallized from ethanol/water. (0.709 g, 72%): ¹H NMR (CDCl₃/300 MHz) 8.02 (s, 1H), 6.78 (s, 1H) 4.95 (s, 1H) 4.68 (t, 2H), 4.35 q, 2H) 3.92 (s, 3H), 3.61 (t, 2H), 3.04-2.88 (m, 4H), 1.42 (t, 3H), 1.41 (s, 9H). ESHRMS m/z 494.1272 (M+H, C₂₂H₂₈BrN₃O₅ requires 494.1285).

Step 2: Preparation of 8-bromo-2-{2-[(tert-butoxy-carbonyl)amino]ethyl}-7-methoxy-4,5-dihydro-2H-benzo-[g]indazole-3-carboxylic Acid.

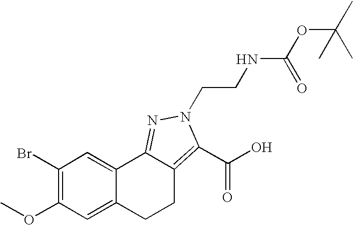

8-bromo-2-{2-[(tert-butoxycarbonyl)amino]ethyl}-7-methoxy-4,5-dihydro-2H-benzo[g]indazole-3-carboxylate (0.494, 1.0 mmol) was dissolved in a THF/ethanol/water mixture (10 mL, 7/2/1 respectively). Lithium hydroxide monohydrate (0.105 g, 2.50 mmol) was added. Stirred overnight at room temperature. Diluted with ethyl acetate (50 mL) and extracted with 0.1N HCl (2×25 mL), and brine (25 mL). The organic phase was dried over MgSO₄, filtered, and evaporated. The resulting white solid was recrystallized from ethyl acetate/hexane. (0.350 g, 75%): ¹H NMR (DMSO tfa/300 MHz) 7.82 (s, 1H), 7.11 (s, 1H), 6.92 (s, 1H), 4.55 (t, 2H), 3.90 (s, 3H), 3.36 (t, 2H), 2.93-2.78 (m, 4H), 1.33 (s, 9H). ESHRMS m/z 466.0991 (M+H, C₂₀H₂₄BrN₃O₅ requires 466.0972).

Step 3: Preparation of 2-(2-aminoethyl)-8-bromo-7-hydroxy-4,5-dihydro-2H-benzo[g]indazole-3-carboxylic Acid Trifluoroacetate.

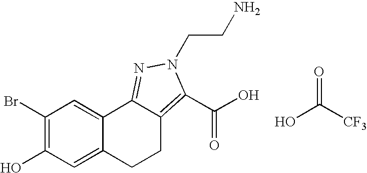

8-bromo-2-{2-[(tert-butoxy-carbonyl)amino]ethyl}-7-methoxy-4,5-dihydro-2H-benzo-[g]indazole-3-carboxylic acid (0.250 g, 0.537 mmol was slurried in CH₂Cl₂ (10 mL). The mixture was cooled to −20° C. Boron tribromide (3 mL, 3 mmol of a 1.0 M solution in CH₂Cl₂) was added dropwise via syringe. The mixture stirred at room temperature for three hours. Slowly added H₂O dropwise to quench the reaction. The solvent was evaporated and the resulting solid was purified by preparative reverse phase HPLC to obtain a white solid (0.170 g): ¹H NMR (DMSO tfa/300 MHz) 7.92 (s, 2H), 7.86 (s, 1H), 6.92 (s, 1H), 4.74 (t, 2H, 3.34 (t, 2H), 2.95-2.83 (m, 4H). ESHRMS m/z 352.0282 (M+H, C₁₄H₁₄BrN₃O₃ requires 352.0291).

### EXAMPLE 89

This example illustrates the production of methyl 8-[(dimethylamino)sulfonyl]-7-hydroxy-4,5-dihydro-2H-benzo[g]indazole-3-carboxylate.

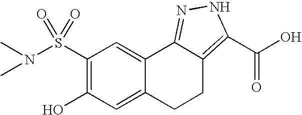

Step 1: Preparation of Methyl (2Z)-hydroxy[6-[(2-methoxyethoxy)methoxy]-1-oxo-3,4-dihydronaphthalen-2(1H)-ylidene]ethanoate.

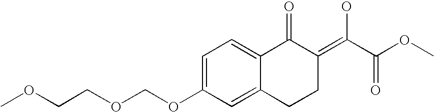

6-Hydroxy-1-tetralone (15.0 g, 92 mmol) was dissolved in THF (200 mL). Lithium-t-butoxide (92 mL, 92 mmol) was slowly added via addition funnel. Stirred for one hour. MEM chloride (12.1 mL, 92 mmol) was addied via addition funnel. Stirred overnight at room temperature. The reaction mixture was washed with H₂O (150 mL), and brine (150 mL). The organic phase was dried over MgSO₄, filtered, and evaporated. Purified by flash column chromatography with 15% ethyl acetate/85% hexane. This compound (15.89 g, 63.6 mmol) was dissolved in ether (300 mL) Dimethyl oxalate (7.51 g, 63.6 mmol) was added. Sodium methoxide (14.53 mL, 63.6 mmol of a 25%/wt. solution in methanol) was slowly added via an addition funnel. Stirred overnight at room temperature. The reaction mixture was treated with 1M HCl to pH=1. The solution was then transferred to a separatory funnel, and the layers were separated. The aqueous phase was extracted with ethyl acetate (2×100 mL). The combined organic phases were washed with brine, dried over MgSO₄, filtered, and evaporated to yield a brown oil, which solidified upon standing. Filtered through a plug of silica with 20% ethyl acetate/80% hexane. The resulting solid was recrystallized from ethyl acetate/hexane. (19.9 g, 64%): ¹H NMR (CDCl₃/300 MHz) 15.92 (s, 1H), 7.97 (d, 1H), 7.01 (dd, 1H), 6.90 (d, 1H), 5.33 (s, 2H), 3.92 (s, 3H), 3.83 (t, 2H), 3.56 (t, 2H), 3.38 (s, 3H), 2.99-2.83 (m 4H). ESHRMS m/z 337.1296 (M+H, C₁₇H₂₀O₇ requires 337.1282)

Step 2: Preparation of Methyl 7-hydroxy-4,5-dihydro-2H-benzo[g]indazole-3-carboxylate.

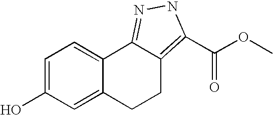

Methyl (2Z)-hydroxy[6-[(2-methoxyethoxy)methoxy]-1-oxo-3,4-dihydronaphthalen-2(1H)-ylidene]ethanoate (19.7 g, 58.6 mmol) was dissolved in methanol (250 mL). Hydrazine monohydrochloride (4.41 g, 64.4 mmol) was added. Heated at reflux for three hours. Cooled to room temperature and neutralized with NaHCO₃ (aq.). Water was added, resulting in the formation of a precipitate. Filtered and washed with H₂O. (10.47 g, 73%): ¹H NMR (acetone/300 MHz) 12.85 (s, 1H), 8.47 (s, 1H), 7.68 (s, 1H), 6.84-6.81 (m 2H), 3.93 (s, 3H), 2.96-2.88 (m 4H). ESHRMS m/z 245.0894 (M+H, C₁₃H₁₂N₂O₃ requires 245.0921).

Step 3: Preparation of Methyl 8-[(dimethylamino)sulfonyl]-7-hydroxy-4,5-dihydro-2H-benzo[g]indazole-3-carboxylate.

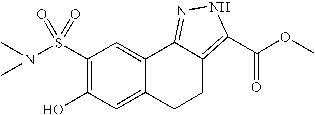

Chlorosulfonic acid (5 mL) was cooled to 0° C. Methyl 7-hydroxy-4,5-dihydro-2H-benzo[g]indazole-3-carboxylate (0.500 g, 2.05 mmol) was added in portions. Stirred at room temperature for two hours. The reaction mixtures was added dropwise to ice. Extracted with ethyl acetate (3×50 mL). The combined organic phases were washed with brine, dried over MgSO₄, filtered, and evaporated to 50 mL. Dimethylamine (40% by weight) was added until the reaction mixture was basic. Stirred overnight at room temperature. Neutralized to pH=7 with 1N HCl. The reaction mixture was extracted with water (50 mL) and brine (50 mL). The organic phase was dried over MgSO₄, filtered, and evaporated to yield a tan solid. Recrystallized from ethyl acetate/hexane (0.160 g, 22%): ¹H NMR (acetone/300 MHz) 12.95 (s, 1H), 9.16 (s, 1H), 8.05 (s, 1H), 7.10 (s 1H), 3.94 (s, 3H), 3.07-3.01 (m 4H) 2.10 (s, 6H). ESHRMS m/z 352.0995 (M+H, C₁₅H₁₇N₃O₅S requires 352.0296)

### EXAMPLE 90

This example illustrates the production of 2-(3-aminopropyl)-7-hydroxy-2H-benzo[g]indazole-3-carboxylic acid trifluoroacetate.

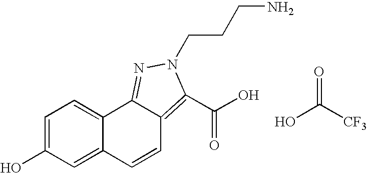

Step 1: Preparation of Methyl (2Z)-hydroxy(6-methoxy-1-oxo-3,4-dihydronaphthalen-2(1H)— ylidene)ethanoate.

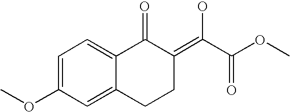

6-Methoxy-1-tetralone (10.0 g, 56.7 mmol) was dissolved in ether (250 mL). Dimethyl oxalate (7.37 g, 62.4 mmol) was added. Sodium methoxide (14.27 mL, 13.48 g, 62.4 mmol of a 25%/wt. solution in methanol) was slowly added via an addition funnel. Stirred overnight at room temperature. The reaction mixture was treated with 1M HCl to pH=1. The solution was then transferred to a separatory funnel, and the layers were separated. The aqueous phase was extracted with ethyl acetate (2×100 mL). The combined organic phases were washed with brine, dried over MgSO₄, filtered, and evaporated to yield a brown oil, which solidified upon standing. Filtered through a plug of silica with 20% ethyl acetate/80% hexane. The resulting solid was recrystallized from ethyl acetate/hexane. (10.19 g, 69%): mp 77.3-77.8° C. ¹H NMR (CDCl₃/300 MHz) 16.02 (s, 1H), 8.03 (d, 1H,), 6.92 (d, 1H), 6.77 (s, 1H), 3.96 (s, 1H), 3.92 (s, 3H), 3.04-2.89 (m, 4H). ESHRMS m/z 263.0907 (M+H, C₁₄H₁₄O₅ requires 263.0914).

Step 2: Preparation of Methyl 7-methoxy-4,5-dihydro-2H-benzo[g]indazole-3-carboxylate,

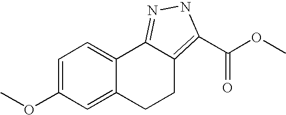

The product from Step 1 (8.77 g, 33.4 mmol) was dissolved in methanol (150 mL). Hydrazine monohydrochloride (2.52 g, 36.8 mmol) was added. Heated at reflux for three hours. Cooled to room temperature and neutralized with NaHCO₃ (aq.). Water was added, resulting in the formation of a precipitate. Filtered and washed with H₂O. The solid was dried in a vacuum oven. (8.4 g, 98%): ¹H NMR (CDCl₃/300 MHz) 10.71 (s, 1H), 7.74 (s, 1H), 6.84-6.82 (m, 2H), 3.94 (s, 1H), 3.83 (s, 1H), 3.05-2.95 (m, 4H). ESHRMS m/z 259.1097 (M+H, C₁₄H₁₄N₂O₃ requires 259.1077).

Step 3: Preparation of Methyl 2-{3-[(tert-butoxy-carbonyl)amino]propyl}-7-methoxy-4,5-dihydro-2H-benzo[g]indazole-3-carboxylate,

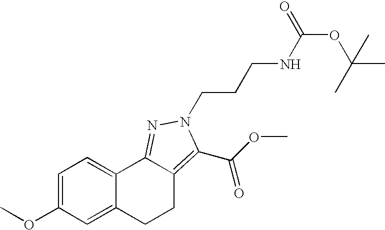

Methyl 7-methoxy-4,5-dihydro-2H-benzo[g]indazole-3-carboxylate (7.00 g, 27 mmol) was dissolved in DMF (100 mL). Cooled to 0° C., in an ice-water bath. Lithium t-butoxide (32 mL, 32 mmol of a 1.0 M solution in THF) was added dropwise via addition funnel. Stirred for 2 hours. N-boc-3-bromopropylamine (7.62 g, 32 mmol) in DMF (20 mL) was added via addition funnel. The ice bath was removed and the reaction stirred at room temperature overnight. Quenched with NH₄Cl (aq.) (200 mL), and the layers were separated. The aqueous phase was extracted with ethyl acetate (2×200 mL). The combined organic phases were washed with brine, (200 mL), dried over MgSO₄, filtered, and evaporated. The resulting brown oil was purified by flash column chromatography in 20% ethyl acetate/80% hexane. The orange solid was recrytstallized from ether/hexane to afford a white solid (8.11 g, 72%): ¹H NMR (CDCl3/300 MHz) 7.75 (d, 1H), 6.83-6.79 (m, 2H), 5.10 (s, 1H), 4.63 (, 2H), 3.91 (s, 3H), 3.83 (s, 3H), 3.13-2.92 (m, 6H), 2.04 (t, 2H), 1.44 (s, 9H). ESHRMS m/z 416.2177 (M+H, C₂₂H₂₉N₃O₅ requires 416.2180).

Step 4: Preparation of 1-methyl 2-{3-[(tert-butoxycarbonyl)amino]propyl}-7-methoxy-2H-benzo[g]indazole-3-carboxylate.

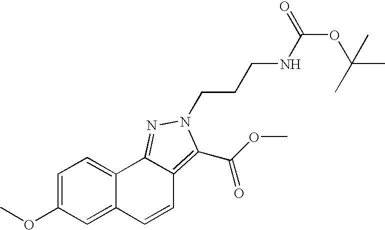

Methyl 2-{3-[(tert-butoxycarbonyl)amino]propyl}-7-methoxy-4,5-dihydro-2H-benzo[g]indazole-3-carboxylate (3.0 g, 7.22 mmol) was dissolved in anhydrous dioxane (50 mL). DDQ (2.05 g, 9.03 mmol) was added. Stirred at reflux for 10 hours. Stirred at room temperature overnight. The resulting solid was filtered, and washed with ethyl acetate (100 mL). The mother liquor was extracted with 1.0 N NaOH until the yellow color dissipated. Checked combined aqueous extracts by TLC for any product. Back extracted with ethyl acetate until the entire product was in organic extracts. The combined organic phases were washed with brine, dried over MgSO₄, filtered and evaporated to yield an orange oil. The oil was filtered through a plug of silica with 20% ethyl acetate/80% hexane. Obtained a clear oil, which solidified upon standing. Recrystallized from ether/hexane (1.94 g, 65%). ¹H NMR (CDCl₃/300 MHz) 8.50 (d, 1H), 7.88 (d, 1H), 7.50 (d, 1H), 7.28-7.25 (m, 2H), 5.19 (s, 1H), 4.96 (t, 2H), 4.05 (s, 3H), 3.95 (s, 3H), 3.14 (t, 2H), 2.18 (qt, 2H), 1.44 (s, 9H). ESHRMS m/z 414.2003 (M+H, C₂₂H₂₇N₃O₅ requires 414.2023).

Step 5: Preparation of 2-{3-[(tert-butoxy-carbonyl)amino]propyl}-7-methoxy-2H-benzo[g]indazole-3-carboxylic Acid.

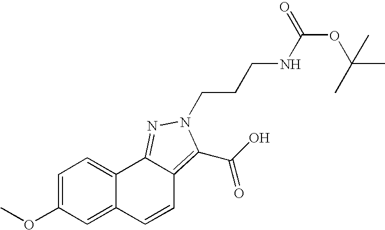

1-methyl 2-{3-[(tert-butoxycarbonyl)amino]propyl}-7-methoxy-2H-benzo[g]indazole-3-carboxylate (0.450 g, 1.09 mmol) was dissolved in a THF/ethanol/water mixture (10 mL, 7/2/1 respectively). Lithium hydroxide monohydrate (0.091 g, 2.18 mmol) was added. Stirred overnight at room temperature. Diluted with ethyl acetate (50 mL) and extracted with 0.1N HCl (2×25 mL), and brine (25 mL). The organic phase was dried over MgSO₄, filtered, and evaporated. The resulting white solid was recrystallized from ethyl acetate/hexane. (0.433 g, 99%): ¹H NMR (DMSO tfa/300 MHz) 8.36 (d, 1H), 7.88 (d, 1H), 7.58 (d, 1H), 7.45 (s, 1H), 7.26 (d, 1H), 6.86 (s, 1H), 4.83 (t, 2H), 3.88 (s, 3H), 2.99 (t, 2H), 2.01 (qt, 2H), 1.35 (s, 9H). ESHRMS m/z 400.1874 (M+H, C₂₁H₂₅N₃O₅ requires 400.1867).

Step 6: Preparation of 2-(3-aminopropyl)-7-hydroxy-2H-benzo[g]indazole-3-carboxylic Acid Trifluoroacetate.

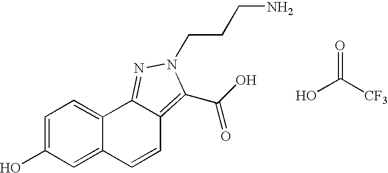

2-{3-[(tert-butoxycarbonyl)amino]propyl)-7-methoxy-2H-benzo[g]indazole-3-carboxylic acid (0.200 g, 0.501 mmol) was slurried in CH₂Cl₂ (5 mL). The mixture was cooled to −20° C. Boron tribromide (5 mL, 5 mmol of a 1.0 M solution in CH₂Cl₂) was added dropwise via syringe. The mixture stirred at room temperature for three hours. Slowly added H₂O dropwise to quench the reaction. The solvent was evaporated and the resulting solid was purified by preparative reverse phase HPLC to obtain a white solid: ¹H NMR (DMSO tfa/300 MHz) 8.28 (d, 1H), 7.82 (d, 1H), 7.70 (s, 1H), 7.48 (d, 1H), 7.22 (d, 1H), 7.14 (dd, 1H), 4.92 (t, 2H), 2.84 (t, 2H), 2.20 (qt, 2H), 1.35 (s, 9H). ESHRMS m/z 286.1148 (M+H, C₁₅H₁₅N₃O₃ requires 286.1186)

### EXAMPLE 91

This example illustrates the production of 2-{3-[(tert-butoxycarbonyl)amino]propyl}-8-(4-chlorophenyl)-7-methoxy-4,5-dihydro-2H-benzo[g]indazole-3-carboxylic acid.

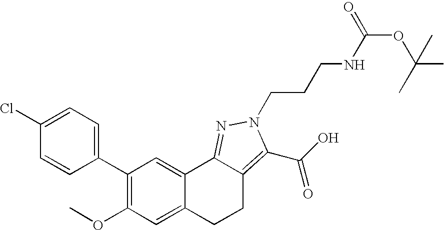

Step 1: Preparation of Methyl 8-bromo-7-methoxy-4,5-dihydro-2H-benzo[g]indazole-3-carboxylate.

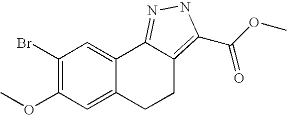

Methyl 7-methoxy-4,5-dihydro-2H-benzo[g]indazole-3-carboxylate (5.36 g, 20.8 mmol) was dissolved in glacial acetic acid (100 mL), and sodium acetate (1.88 g, 22.9 mmol was added. Cooled to 15° C. in an ice-water bath, and added bromine (22.9 mL of a 1.0 M solution in acetic acid) was added via addition funnel. The ice bath was removed, and the reaction stirred at room temperature for three hours. The acetic acid was evaporated, and the resulting solid was dissolved in ethyl acetate (300 mL). Extracted with NaHCO₃ (aq.) (2×100 mL) and brine (100 mL). The organic phase was dried over MgSO₄, filtered, and evaporated. The resulting solid was recrystallized from ethyl acetate/hexane to afford a white solid. (4.7 g, 66%) mp 239.7-241.4° C.: ¹H NMR (CDCl₃/300 MHz) 8.04 (s, 1H), 6.80 (s, 1H), 3.95 (s, 3H), 3.92 (s, 3H), 3.06-2.94 (m, 4H). ESHRMS m/z 337.0195 (M+H, C₁₄H₁₃BrN₂O₃ requires 337.0182).

Step 2: Preparation of Methyl 8-bromo-2-{3-[(tert-butoxycarbonyl)amino]propyl}-7-methoxy-4,5-dihydro-2H-benzo[g]indazole-3-carboxylate.

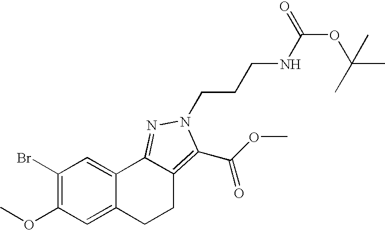

Methyl 8-bromo-7-methoxy-4,5-dihydro-2H-benzo[g]indazole-3-carboxylate (10.10 g, 30 mmol) was dissolved in DMF (150 mL). Cooled to 0° C., in a ice-water bath. Lithium t-butoxide (50 mL, 50 mmol of a 1.0 M solution in THF) was added dropwise via addition funnel. Stirred for 2 hours. N-boc-3-bromopropylamine (10.69 g, 45 mmol) in DMF (25 mL) was added via addition funnel. The ice bath was removed and the reaction stirred at room temperature overnight. Quenched with NH₄Cl (aq.) (200 mL), and the layers were separated. The aqueous phase was extracted with ethyl acetate (2×200 mL). The combined organic phases were washed with brine, (200 mL), dried over MgSO₄, filtered, and evaporated. The resulting orange solid was recrystallized from hot ethanol/water to yield a white solid, which was dried under vacuum. (10.57 g, 72%): ¹H NMR (CDCl₃/300 MHz) 8.00 (s, 1H), 6.78 (s, 1H), 4.99 (s, 1H) 4.63 (t, 2H), 3.92 (s, 3H), 3.12 (t, 2H), 2.99-2.88 (m, 4H), 2.04 (qt, 2H), 1.45 (s, 9H). ESHRMS m/z 494.1267 (M+H, C₂₂H₂₈BrN₃O₅ requires 494.1285).

Step 3: Preparatio of Methyl-2-{3-[(tert-butoxycarbonyl)amino]propyl}-8-(4-chlorophenyl)-7-methoxy-4,5-dihydro-2H-benzo[g]indazole-3-carboxylate.

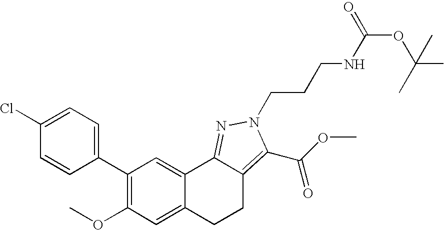

Methyl 8-bromo-2-{3-[(tert-butoxycarbonyl)amino]propyl}-7-methoxy-4,5-dihydro-2H-benzo[g]indazole-3-carboxylate (1.00 g, 2.02 mmol), and 4-chlorophenyl boronic acid (0.632 g, 4.04 mmol) were slurried in 1-propanol (40 mL). Sodium carbonate (0.428 g, 4.06 mmol) in water (2 mL) was added, followed by palladium acetate (0.0136 g, 0.06 mmol), and triphenyl phosphine (0.0477 g, 0.182 mmol). The reaction mixture was heated to reflux for two hours. (Reaction mixture became homogeneous upon heating). The reaction cooled to room temperature, and the propanol was evaporated to approximately 5 mL. The slurry was diluted with ethyl acetate (100 mL). Extracted with H₂O (50 mL), and brine (50 mL). The organic phase was dried over MgSO₄, filtered and evaporated. Purified by flash column chromatography in 15% ethyl acetate/85% hexane. The resulting solid was recrystallized from ethanol/water. (0.632 g, 60%): ¹H NMR (CDCl₃/300 MHz) 7.76 (s, 1H), 7.51 (d, 2H), 7.35 (d, 2H), 6.86 (s, 1H), 4.99 (s, 1H), 4.63 (t, 2H), 3.92 (s, 3H), 3.83 (s, 3H), 3.06 (t, 2H), 3.02-2.96 (m, 4H), 2.04 (qt, 2H), 1.42 (s, 9H). ESHRMS m/z 526.2097 (M+H, C₂₈H₃₂ClN₃O₅ requires 526.2103).

Step 4: Preparation of 2-{3-[(tert-butoxycarbonyl)amino]propyl}-8-(4-chlorophenyl)-7-methoxy-4,5-dihydro-2H-benzo[g]indazole-3-carboxylic Acid.

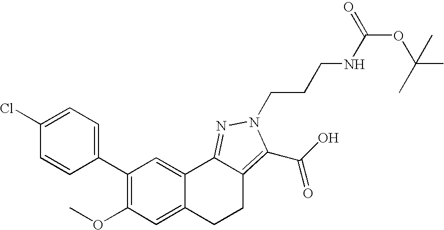

Methyl-2-{3-[(tert-butoxycarbonyl)amino]propyl}-8-(4-chlorophenyl)-7-methoxy-4,5-dihydro-2H-benzo[g]indazole-3-carboxylate (0.526 g, 1.00 mmol) was dissolved in a THF/ethanol/water mixture (10 mL, 7/2/1 respectively). Lithium hydroxide monohydrate (0.105 g, 2.5 mmol was added. Stirred overnight at room temperature. Diluted with ethyl acetate (50 mL) and extracted with 0.1N HCl (2×25 mL), and brine (25 mL). The organic phase was dried over MgSO₄, filtered, and evaporated. The resulting white solid was recrystallized from ethyl acetate/hexane. (0.409 g, 80%): ¹H NMR (CDCl₃/300 MHz) 7.77 (s, 1H), 7.51 (d, 2H), 7.36 (d, 2H), 6.87 (s, 1H), 5.01 (s, 1H), 4.65 (t, 2H), 3.84 (s, 3H), 3.17-2.99 (m, 6H), 2.06 (qt, 2H), 1.42 (s, 9H). ESHRMS m/z 512.1971 (M+H, C₂₇H₃₀ClN₃O₅ requires 512.1947).

Following the procedure described for the production of 2-{3-[(tert-butoxycarbonyl)amino]propyl}-8-(4-chlorophenyl)-7-methoxy-4,5-dihydro-2H-benzo[g]indazole-3-carboxylic acid, the subsequent compounds were prepared.

### EXAMPLE 92

This example illustrates the production of 2-{3-[(tert-butoxycarbonyl) amino]propyl}-7-methoxy-8-(3-nitrophenyl)-4,5-dihydro-2H-benzo[g]indazole-3-carboxylic acid.

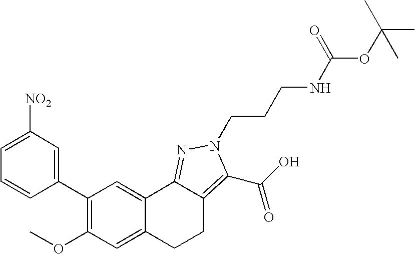

¹H NMR (DMSO tfa/300 MHz) 8.33 (s, 1H), 8.22 (d, 1H), 8.00 (d, 1H), 7.76-7.68 (m, 2H), 7.17 (s, 1H), 6.83 (s, 1H), 4.52 (t, 2H), 3.86 (s, 3H), 3.05-2.95 (m, 6H), 1.90 (qt, 2H), 1.37 (s, 9H). ESHRMS m/z 523.2186 (M+H, C₂₇H₃₀N₄O₇ requires 523.2187)

### EXAMPLE 93

This example illustrates the production of 2-{3-[(tert-butoxycarbonyl) amino]propyl}-8-(2-chlorophenyl)-7-methoxy-4,5-dihydro-2H-benzo[g]indazole-3-carboxylic acid.

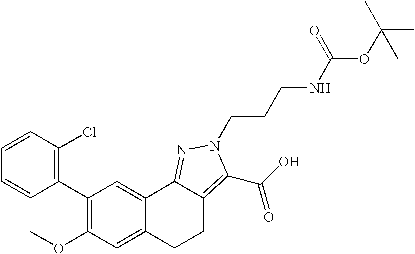

¹H NMR (DMSO tfa/300 MHz) 7.51 (s, 1H), 7.43-7.37 (m, 4H), 7.09 (s, 1H), 6.81 (s, 1H), 4.49 (t, 2H), 3.77 (s, 3H), 2.99-2.93 (m, 6H), 1.88 (qt, 2H), 1.36 (s, 9H). ESHRMS m/z 512.1928 (M+H, C₂₇H₃₀ClN₃O₅ requires 512.1947)

### EXAMPLE 94

This example illustrates the production of 2-{3-[(tert-butoxycarbonyl) amino]propyl}-8-[1-(tert-butoxycarbonyl)-1H-pyrrol-2-yl]-7-methoxy-4,5-dihydro-2H-benzo[g]indazole-3-carboxylic acid.

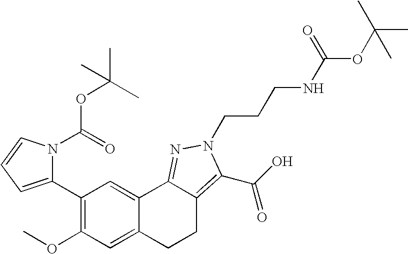

¹H NMR (DMSO tfa/300 MHz) 7.51 (s, 1H), 7.31 (q, 4H), 6.97 (s, 1H), 6.83 (s, 1H), 6.26 (t, 1H), 6.19 (s, 1H), 4.50 (t, 2H), 3.73 (s, 3H), 3.03-2.93 (m, 6H), 1.88 (qt, 2H), 1.38 (s, 9H), 1.32 (s, 9H). ESHRMS m/z 567.2788 (M+H, C₃₀H₃₈N₄O₇ requires 567.2813)

### EXAMPLE 95

This example illustrates the production of 2-{3-[(tert-butoxycarbonyl)amino]propyl}-8-(3-chlorophenyl)-7-methoxy-4,5-dihydro-2H-benzo[g]indazole-3-carboxylic acid,

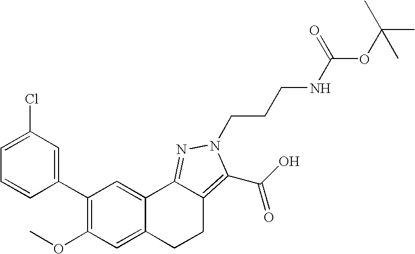

¹H NMR (DMSO tfa/300 MHz) 7.59 (s, 1H), 7.55 (s, 1H), 7.49-7.47 (m, 2H), 7.45-7.40 (m, 1H), 7.12 (s, 1H), 6.83 (s, 1H), 4.51 (t, 2H), 3.83 (s, 3H), 3.03-2.95 (m, 6H), 1.90 (qt, 2H), 1.37 (s, 9H). ESHRMS m/z 512.1933 (M+H, C₂₇H₃₀ClN₃O₅ requires 567.2813)

### EXAMPLE 96

This example illustrates the production of 2-{3-[(tert-butoxycarbonyl)amino]propyl}-8-(3-cyanophenyl)-7-methoxy-4,5-dihydro-2H-benzo[g]indazole-3-carboxylic acid.

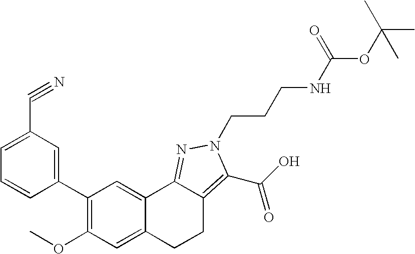

¹H NMR (DMSO tfa/300 MHz) 7.96 (s, 1H), 7.88-7.80 (m, 2H), 7.67-7.62 (m, 2H), 7.14 (s, 1H), 6.83 (s, 1H), 4.51 (t, 2H), 3.84 (s, 3H), 2.99-2.90 (m, 6H), 1.91 (qt, 2H), 1.37 (s, 9H). ESHRMS m/z 503.2259 (M+H, C₂₈H₃₀ClN₄O₅ requires 503.2289)

### EXAMPLE 97

This example illustrates the production of 2-{3-[(tert-butoxycarbonyl)amino]propyl}-7-methoxy-8-(4-nitrophenyl)-4,5-dihydro-2H-benzo[g]indazole-3-carboxylic acid.

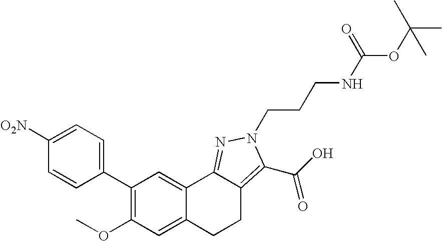

¹H NMR (DMSO tfa/300 MHz) 8.26 (d, 2H), 7.80 (d, 2H), 7.64(s, 1H), 7.15 (s, 1H), 6.81 (s, 1H), 4.48 (t, 2H), 3.82 (s, 3H), 3.06-2.96 (m, 6H), 1.87 (qt, 2H), 1.34 (s, 9H). ESHRMS m/z 523.2212 (M+H, C₂₇H₃₀N₄O₇ requires 523.2187).

### EXAMPLE 98

This example illustrates the production of 2-{3-[(tert-butoxycarbonyl) amino]propyl}-7-methoxy-8-[3-(methylsulfonyl)phenyl]-4,5-dihydro-2H-benzo[g]indazole-3-carboxylic acid.

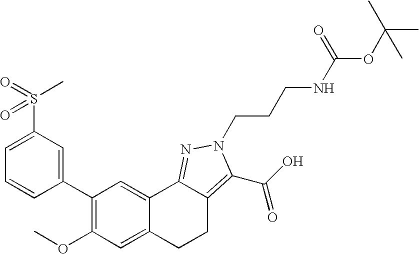

¹H NMR (DMSO tfa/300 MHz) 8.01 (d, 1H), 7.88-7.85(m, 2H), 7.68(d, 1H), 7.64 (s, 1H), 7.11 (s, 1H), 6.81 (s, 1H), 4.49 (t, 2H), 3.81 (s, 3H), 3.25 (s, 3H), 2.96-2.88 (m, 6H), 1.88 (qt, 2H), 1.33 (s, 9H). ESHRMS m/z 556.2137 (M+H, C₂₈H₃₃N₃O₇S requires 556.2112).

### EXAMPLE 99

This example illustrates the production of 2-{3-[(tert-butoxycarbonyl) amino]propyl}-8-(3,4-difluorophenyl)-7-methoxy-4,5-dihydro-2H-benzo[g]-indazole-3-carboxylic acid

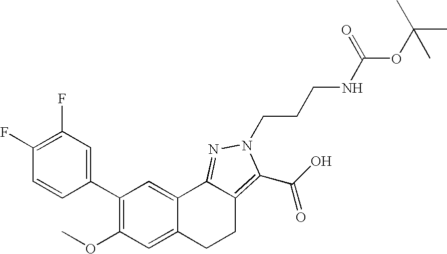

¹H NMR (DMSO tfa/300 MHz) 7.57 (s, 1H), 7.55-7.32(m, 2H), 7.09(s, 1H), 6.80 (s, 1H), 4.48 (t, 2H), 3.80 (s, 3H), 2.95-2.82 (m, 6H), 1.85 (qt, 2H), 1.34 (s, 9H). ESHRMS m/z 514.2170 (M+H, C₂₇H₂₉F₂N₃O₅ requires 556.2112)

### EXAMPLE 100

This example illustrates the production of 2-(3-aminopropyl)-8-(4-chlorophenyl)-7-hydroxy-4,5-dihydro-2H-benzo[g]indazole-3-carboxylic acid trifluoroacetate.

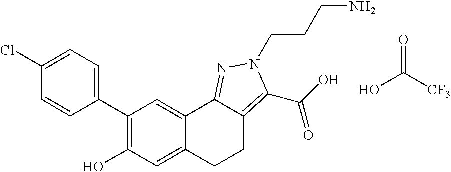

2-{3-[(tert-butoxycarbonyl)amino]propyl}-8-(4-chlorophenyl)-7-methoxy-4,5-dihydro-2H-benzo[g]indazole-3-carboxylic acid (0.300 g, 0.586 mmol) was slurried in CH₂Cl₂ (15 mL). The mixture was cooled to −20° C. Boron tribromide (6 mL, 6 mmol of a 1.0 M solution in CH₂Cl₂) was added dropwise via syringe. The mixture was stirred at room temperature for three hours. Then, H₂O was slowly added dropwise to quench the reaction. The solvent was evaporated and the resulting solid was purified by preparative reverse phase HPLC to yield a light yellow solid (0.094 g): ¹H NMR (DMSO tfa/300 MHz) 7.81 (s, 2H), 7.72 (s, 1H), 7.60 (d, 2H), 7.49 (d, 2H), 6.90 (s, 1H), 4.59 (t, 2H), 2.96-2.84 (m, 6H), 2.11-2.08 (qt, 2H). ESHRMS m/z 398.1262 (M+H, C₂₁H₂₀ClN₃O₃ requires 398.1266).

Following the procedure described for 2-(3-aminopropyl)-8-(4-chlorophenyl)-7-hydroxy-4,5-dihydro-2H-benzo[g]indazole-3-carboxylic acid trifluoroacetate, the subsequent 13 compounds were prepared.

### EXAMPLE 101

This example illustrates the production of 2-(3-Aminopropyl)-7-hydroxy-8-[4-(methylamino)phenyl]-4,5-dihydro-2H-benzo[g]indazole-3-carboxylic acid trifluoroacetate

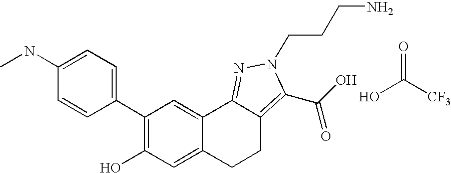

¹H NMR (DMSO tfa/300 MHz) 7.73 (s, 1H), 7.63 (d, 2H), 7.59 (s, 1H), 7.35 (d, 2H), 6.89 (s, 1H), 4.58 (t, 2H), 3.14 (s, 3H) 2.96-2.84 (m, 6H), 2.11-2.08 (qt, 2H). ESHRMS m/z 393.1945 (M+H, C₂₂H₂₄N₄O₃ requires 393.1921).

### EXAMPLE 102

This example illustrates the production of 2-(3-Aminopropyl)-8-(4-tert-butylphenyl)-7-hydroxy-4,5-dihydro-2H-benzo[g]indazole-3-carboxylic acid trifluoroacetate.

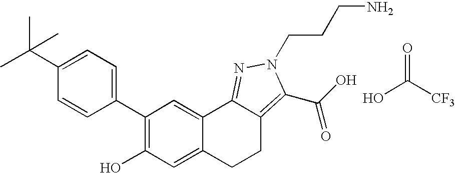

¹H NMR (DMSO ffa/300 MHz) 7.70 (s, 2H), 7.59 (s, 1H), 7.50 (d, 2H), 7.44 (d, 2H), 6.88 (s, 1H), 4.59 (t, 2H), 2.96-2.84 (m, 6H), 2.10-2.05 (qt, 2H), 1.34 (s, 9H). ESHRMS m/z 420.2275 (M+H, C₂₅H₂₉N₃O₃ requires 420.2282).

### EXAMPLE 103

This example illustrates the production of 2-(3-Aminopropyl)-7-hydroxy-8-[3-(trifluoromethyl)phenyl]-4,5-dihydro-2H-benzo[g]indazole-3-carboxylic acid trifluoroacetate.

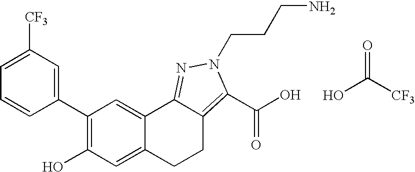

¹H NMR (DMSO tfa/300 MHz) 7.89 (s, 2H), 7.70-7.57 (m, 5H), 6.93 (s, 1H), 4.59 (t, 2H), 3.00-2.84 (m, 6H), 2.07(qt, 2H). ESHRMS m/z 432.1551 (M+H, C₂₂H₂OF₃N₃O₃ requires 432.1530).

### EXAMPLE 104

This example illustrates the production of 2-(3-Aminopropyl)-7-hydroxy-8-pyridin-4-yl-4,5-dihydro-2H-benzo[g]indazole-3-carboxylic acid trifluoroacetate.

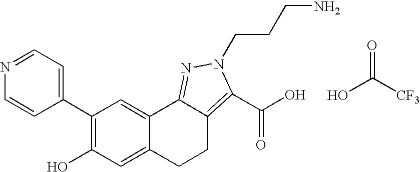

¹H NMR (DMSO tfa/300 MHz) 8.92 (d, 2H), 8.37 (d, 2H), 7.86 (s, 1H), 7.74 (s, 2H), 7.04 (s, 1H), 4.60 (t, 2H), 2.98-2.83 (m, 6H), 2.09(qt, 2H). ESHRMS m/z 365.1654 (M+H, C₂₀H₂₀N₄O₃ requires 365.1608).

### EXAMPLE 105

This example illustrates the production of 2-(3-aminopropyl)-7-hydroxy-8-(3-nitrophenyl)-4,5-dihydro-2H-benzo[g]indazole-3-carboxylic acid trifluoroacetate.

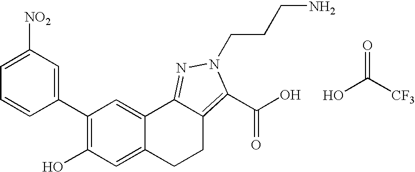

¹H NMR (DMSO tfa/300 MHz) 8.42 (s, 1H), 8.21 (d, 1H), 8.07 (d, 1H), 7.78-7.68 (m, 4H), 6.96 (s, 1H), 4.60 (t, 2H), 2.98-2.82 (m, 6H), 2.09 (qt. 2H). ESHRMS m/z 409.1525 (M+H, C₂₁₅H₂ON₄O₅ requires 409.1506).

### EXAMPLE 106

This example illustrates the production of 2-(3-Aminopropyl)-8-(2-chlorophenyl)-7-hydroxy-4,5-dihydro-2H-benzo[g]indazole-3-carboxylic acid trifluoroacetate.

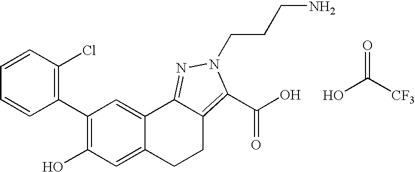

¹H NMR (DMSO tfa/300 MHz) 7.67 (s, 2H), 7.54 (m, 1H), 7.41-7.37 (m, 4H), 6.87 (s, 1H), 4.56 (t, 2H), 2.97-2.81 (m, 6H), 2.07 (qt, 2H). ESHRMS m/z 398.1276 (M+H, C₃₀H₃₆ClN₃O₅ requires 398.1266)

### EXAMPLE 107

This example illustrates the production of 2-(3-Aminopropyl)-8-(3-chlorophenyl)-7-hydroxy-4,5-dihydro-2H-benzo[g]indazole-3-carboxylic acid trifluoroacetate,

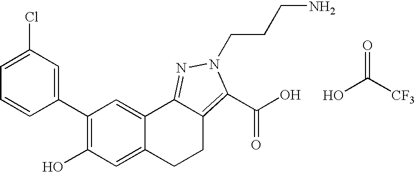

¹H NMR (DMSO tfa/300 MHz) 7.73 (s, 2H), 7.60 (d, 1H) 7.55-7.41 (m, 4H), 6.92 (s, 1H), 4.59 (t, 2H), 2.97-2.85 (m, 6H), 2.09 (qt, 2H). ESHRMS m/z 398.1260 (M+H, C₂₁H₂₀ClN₃O₃ requires 398.1266)

### EXAMPLE 108

This example illustrates the production of 2-(3-Aminopropyl)-8-(3-carboxyphenyl)-7-hydroxy-4,5-dihydro-2H-benzo[g]indazole-3-carboxylic acid trifluoroacetate.

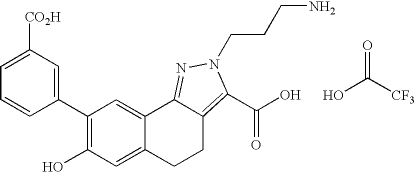

¹H NMR (DMSO ffa/300 MHz) 8.14 (s, 1H), 7.91 (d, 1H) 7.86 (d, 1H), 7.69 (s, 2H), 7.63 (s, 1H), 7.57 (s, 1H), 6.92 (s, 1H), 4.60 (t, 2H), 2.99-2.83 (m, 6H), 2.09 (qt, 2H). ESHRMS m/z 408.1564 (M+H, C₂₂H₂₁N₃O₅ requires 408.1559)

### EXAMPLE 169

This example illustrates the production of 2-(3-Aminopropyl)-8-(3,5-dimethylisoxazol-4-yl)-7-hydroxy-4,5-dihydro-2H-benzo[g]indazole-3-carboxylic acid trifluoroacetate.

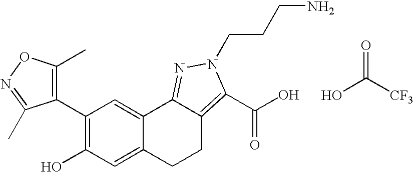

¹H NMR (DMSO tfa/300 MHz) 7.69 (s, 2H), 7.41 (s, 1H) 6.91 (s, 1H), 4.58 (t, 2H), 2.99-2.83 (m, 6H), 2.30 (s, 3H), 2.13 (s, 3H), 2.07 (qt, 2H). ESHRMS m/z 383.1687 (M+H, C₂₀H₂₂N₄O₄ requires 383.1714)

### EXAMPLE 110

This example illustrates the production of 2-(3-Aminopropyl)-8-(3-cyanophenyl)-7-hydroxy-4,5-dihydro-2H-benzo[g]indazole-3-carboxylic acid trifluoroacetate

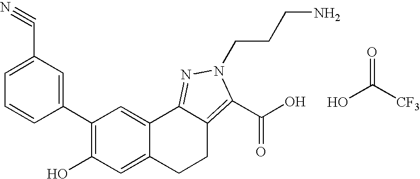

¹H NMR (DMSO tfa/300 MHz) 7.99 (s, 1H), 7.92 (d, 1H) 7.80 (d, 1H), 7.70-7.62 (m, 4H), 6.93 (s, 1H), 4.59 (t, 2H), 2.97-2.82 (m, 6H), 2.08 (qt, 2H). ESHRMS m/z 389.1623 (M+H, C₂₂H₂₀N₄O₃ requires 389.1608)

### EXAMPLE 111

This example illustrates the production of 2-(3-Aminopropyl)-7-hydroxy-8-(4-nitrophenyl)-4,5-dihydro-2H-benzo[g]indazole-3-carboxylic acid trifluoroacetate.

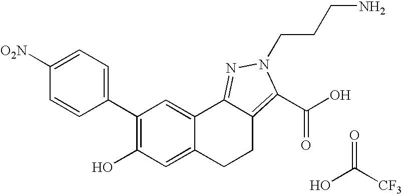

¹H NMR (DMSO tfa/300 MHz) 8.27 (d, 2H), 7.85 (d, 2H) 7.68-7.64 (m, 3H), 6.93 (s, 1H), 4.57 (t, 2H), 2.94-2.81 (m, 6H), 2.08 (qt, 2H). ESHRMS m/z 409.1506 (M+H, C₂₁H₂₀N₄O₅ requires 409.1482)

### EXAMPLE 112

This example illustrates the production of 2-(3-Aminopropyl)-7-hydroxy-8-[3-(methylsulfonyl)phenyl]-4,5-dihydro-2H-benzo[g]indazole-3-carboxylic acid trifluoroacetate.

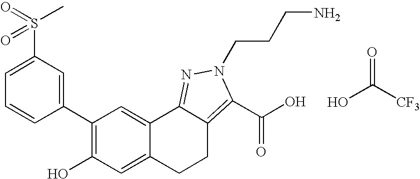

¹H NMR (DMSO tfa/300 MHz) 8.07 (s, 2H), 7.95 (d, 1H) 7.87 (d, 1H), 7.69-7.63 (m, 4H), 6.92 (s, 1H), 4.58 (t, 2H), 3.26 (s, 3H), 2.95-2.82 (m, 6H), 2.06 (qt, 2H). ESHRMS m/z 442.1424 (M+H, C₂₂H₂₃N₃O₅S requires 442.1431)

### EXAMPLE 113

This example illustrates the production of 2-(3-Aminopropyl)-8-(3,4-difluorophenyl)-7-hydroxy-4,5-dihydro-2H-benzo[g]indazole-3-carboxylic acid trifluoroacetate,

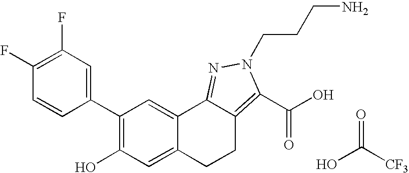

¹H NMR (DMSO tfa/300 MHz) 7.70 (s, 2H), 7.63-7.58(m, 1H), 7.56(s, 1H), 7.48-7.38 (m, 2H), 6.88 (s, 1H), 4.56 (t, 2H), 2.95-2.76 (m, 6H), 2.05 (qt, 2H). ESHRMS m/z 400.1473 (M+H, C₂₁H₁₉F₂N₃O₃ requires 400.1467)

### EXAMPLE 114

This example illustrates the production of 2-{3-[(tert-butoxycarbonyl) amino]propyl}-8-[2-(4-chlorophenyl)ethyl]-7-methoxy-4,5-dihydro-2H-benzo[g]-indazole-3-carboxylic acid,

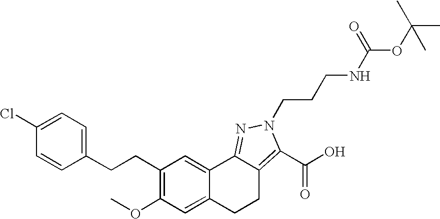

Step 1: Preparation of Methyl 2-{3-[(tert-butoxycarbonyl)amino]propyl}-8-[2-(4-chlorophenyl)ethyl]-7-methoxy-4,5-dihydro-2H-benzo[g]indazole-3-carboxylate.

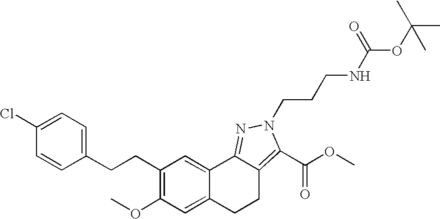

9-BBN (8.1 mL, 4.04 mmol of a 0.5M solution in THF) was added to a 100 mL round bottomed flask under Ar. Cooled to 10° C. in an ice/water bath. 4-Chlorostyrene (0.6 g, 4.04 mmol) was added dropwise via syringe. Stirred at room temperature for four hours. Methyl 8-bromo-2-{3-[(tert-butoxycarbonyl)amino]propyl}-7-methoxy-4,5-dihydro-2H-benzo[g]indazole-3-carboxylate (1.00 g, 2.02 mmol) dissolved into THF (10 mL) was added, followed by Pd{dppf]Cl₂CH₂Cl₂ (0.124 g, 0.101 mmol), and K₃PO₄ (2.28 mL, 4.54 mmol of a 2M solution in H₂O). Heated at 60° C. for 8 hours. Stirred at room temperature overnight. Quenched with NaHCO₃ (aq.) (100 mL). Extracted with ethyl acetate (2×100 mL). The combined organic phases were washed with brine, dried over MgSO₄, filtered, and evaporated. Purified by flash column chromatography in 15% ethyl acetate/85% hexane. The resulting oil solidified upon standing. Recrystallized from ether/hexane (0.607 g, 54%): ¹H NMR (CDCl₃/300 MHz) 7.61 (s, 1H), 7.23-7.14 (m 4H), 6.72 (s, 1H), 4.98 (s, 1H), 4.63 (t, 2H), 3.92 (s, 3H), 3.82 (s, 3H), 3.11 (t, 2H), 2.99-2.88 (m, 8H), 2.05 qt, 2H), 1.44 (s, 9H). ESHRMS m/z 554.2389 M+H, C₃₀H₃₆ClN₃O₅ requires 554.2416).

Step 2: Preparation of 2-{3-[(tert-butoxycarbonyl)amino]propyl}-8-[2-(4-chlorophenyl)ethyl]-7-methoxy-4,5-dihydro-2H-benzo[g]indazole-3-carboxylic acid.

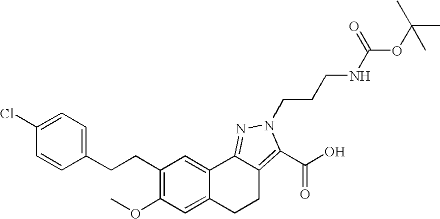

Methyl 2-{3-[(tert-butoxycarbonyl)amino]propyl}-8-[2-(4-chlorophenyl)-ethyl]-7-methoxy-4,5-dihydro-2H-benzo[g]indazole-3-carboxylate (0.400 g, 0.722 mmol) was dissolved in a THF/ethanol/water mixture (10 mL, 7/2/1 respectively). Lithium hydroxide monohydrate (0.060 g, 1.44 mmol was added. Stirred overnight at room temperature. Diluted with ethyl acetate (50 mL) and extracted with 0.1N HCl (2×25 mL), and brine (25 mL). The organic phase was dried over MgSO₄, filtered, and evaporated. The resulting white solid was recrystallized from ethyl acetate/hexane. (0.352 g, 90%): ¹H NMR (DMSO tfa/300 MHz) 7.50 (s, 1H), 7.35-7.26 (m 4H), 6.92 (s, 1H), 6.89 (s, 1H), 4.49 (t, 2H), 3.83 (s, 3H), 3.05-2.91 (m, 10H), 1.89 (qt, 2H), 1.38 (s, 9H). ESHRMS m/z 540.2224 (M+H, C₂₉H₃₄ClN₃O₅ requires 540.2260).

Following the procedure described for the production of 2-{3-[(tert-butoxycarbonyl)amino]propyl}-8-[2-(4-chlorophenyl)ethyl]-7-methoxy-4,5-dihydro-2H-benzo[g]indazole-3-carboxylic acid, the subsequent 2 compounds were prepared.

### EXAMPLE 115

This example illustrates the production of 8-benzyl-2-{3-[(tert-butoxycarbonyl) amino]propyl}-7-methoxy-4,5-dihydro-2H-benzo[g]indazole-3-carboxylic acid.

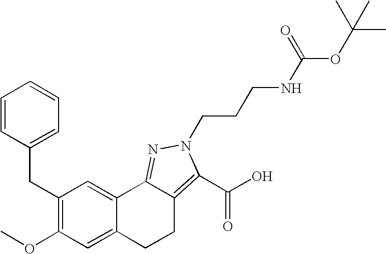

¹H NMR (DMSO tfa/300 MHz) 7.47 (s, 1H), 7.30-7.15 (m 5H), 6.95 (s, 1H), 6.82 (s, 1H), 4.47 (t, 2H), 3.93 (s, 2H), 3.83 (s, 3H), 2.94-2.89 (m, 6H), 1.87 (qt, 2H), 1.39 (s, 9H). ESHRMS m/z 492.2483 (M+H, C₂₈H₃₃N₃O₅ requires 492.2493).

### EXAMPLE 116

This example illustrates the production of 2-{3-[(tert-butoxycarbonyl) amino]propyl}-7-methoxy-8-(3,3,3-trifluoropropyl)-4,5-dihydro-2H-benzo[g]indazole-3-carboxylic acid.

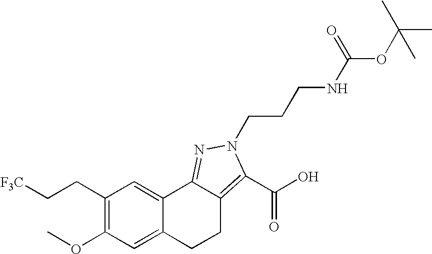

¹H NMR (DMSO tfa/300 MHz) 7.55 (s, 1H), 6.94 (s, 1H), 6.84 (s, 1H), 4.50 (t, 2H), 3.84 (s, 3H), 2.96-2.80 (m, 8H), 2.60-2.52 (m, 2H), 1.90 (qt, 2H), 1.39 (s, 9H). ESHRMS m/z 498.2233 (M+H, C₂₄H₃₀F₃N₃O₅, requires 498.2210).

### EXAMPLE 117

This example illustrates the production of 2-{3-[(tert-butoxycarbonyl) amino]propyl}-8-[2-(4-chlorophenyl)ethyl]-7-methoxy-4,5-dihydro-2H-benzo[g]indazole-3-carboxylic acid (0.275 g, 0.509 mmol) was slurried in CH₂Cl₂ (10 mL).

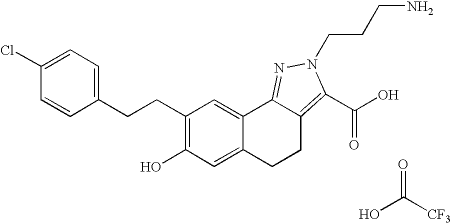

The mixture was cooled to −20° C. Boron tribromide (5 mL, 5 mmol of a 1.0 M solution in CH₂Cl₂) was added dropwise via syringe. The mixture stirred at room temperature for three hours. Slowly added H₂O dropwise to quench the reaction. The solvent was evaporated, and the resulting solid was purified by preparative reverse phase HPLC to yield a white solid (0.170 g): ¹H NMR (DMSO tfa/300 MHz) 7.71 (s, 2H), 7.43 (s, 1H), 7.37-7.27 (m 4H), 6.74 (s, 1H), 4.56 (t, 2H), 2.93-2.80 (m, 10H), 2.08 (qt, 2H. ESHRMS m/z 426.1558 (M+H, C₂₃H₂₄ClN₃O₃ requires 426.1579).

Following the procedure described for the production of 2-(3-aminopropyl)-8-[2-(4-chlorophenyl)ethyl]-7-hydroxy-4,5-dihydro-2H-benzo[g]indazole-3-carboxylic acid trifluoroacetate, the subsequent 4 compounds were prepared.

### EXAMPLE 118

This example illustrates the production of 2-(3-aminopropyl)-8-benzyl-7-hydroxy-4,5-dihydro-2H-benzo[g]indazole-3-carboxylic acid trifluoroacetate.

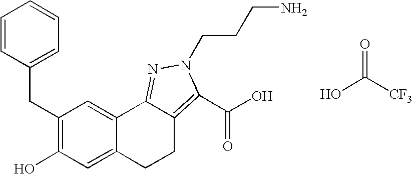

¹H NMR (DMSO tfa/300 MHz) 7.69 (s, 2H), 7.38 (s, 1H), 7.33-7.20 (m 5H), 6.76 (s, 1H), 4.54 (t, 2H), 3.91 (s, 2H), 2.92-2.78 (m, 6H), 2.05 (qt, 2H). ESHRMS m/z 378.1792 (M+H, C₂₂H₂₃N₃O₃, requires 378.1812).

### EXAMPLE 119

This example illustrates the production of 2-(3-aminopropyl)-7-hydroxy-8-isobutyl-4,5-dihydro-2H-benzo[g]indazole-3-carboxylic acid trifluoroacetate

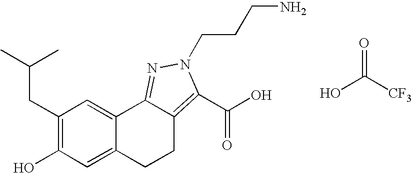

¹H NM R (DMSO tfa/300 MHz) 7.70 (s, 2H), 7.36 (s, 1H), 6.71 (s, 1H), 4.57 (t, 2H), 2.91-2.79 (m 6H), 2.42 (d, 2H), 2.07 (qt, 2H), 1.95 (m, 1H), 0.89 (d, 6H). ESHRMS m/z 344.1957 (M+H, C₁₉H₂₅N₃O₃ requires 344.1969).

### EXAMPLE 120

This example illustrates the production of 2-(3-aminopropyl)-8-(4-carboxy-3,3-dimethylbutyl)-7-hydroxy-4,5-dihydro-2H-benzo[g]indazole-3-carboxylic acid trifluoroacetate.

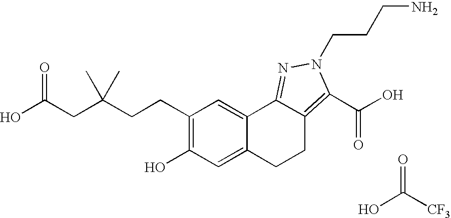

¹H NMR (DMSO tfa/300 MHz) 7.72 (s, 2H), 7.39 (s, 1H), 6.70 (s, 1H), 4.57 (t, 2H), 2.93-2.75 (m, 6H), 2.53-2.49 (m, 2H), 2.20 (s, 2H), 2.08 (qt, 2H), 1.57 (m, 2H), 1.06 (s, 6H). ESHRMS m/z 416.2171 (M+H, C₂₂H₂₉N₃O₅) requires 416.2180)

### EXAMPLE 121

This example illustrates the production of 2-(3-aminopropyl)-7-hydroxy-8-(3,3,3-trifluoropropyl)-4,5-dihydro-2H-benzo[g]indazole-3-carboxylic acid trifluoroacetate.

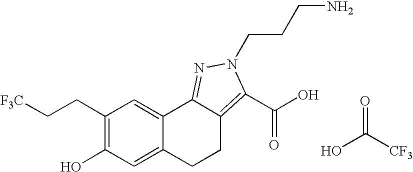

¹H NMR (DMSO tfa/300 MHz) 7.71 (s, 2H), 7.43 (s, 1H), 6.72 (s, 1H), 4.54 (t, 2H), 2.88-2.76 (m, 8H), 2.53-2.44 (m, 2H), 2.05 (qt, 2H). ESHRMS m/z 384.1551 (M+H, C₁₈H₂OF₃N₃O₃ requires 384.1530).

### EXAMPLE 122

This example illustrates the production of 3-hydroxy-2-(3-nitrophenyl)-5,6,8,9,10,11-hexahydro-7H-benzo[g][1,4]diazepino[1,2-b]indazol-7-one trifluoroacetate.

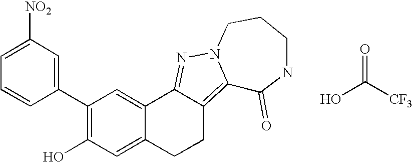

Step 1: Preparation of Methyl 2-{3-[(tert-butoxycarbonyl)amino]propyl}-7-methoxy-8-(3-nitrophenyl)-4,5-dihydro-2H-benzo[g]indazole-3-carboxylate.

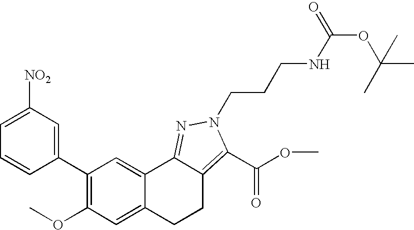

Methyl 8-bromo-2-{3-[(tert-butoxycarbonyl)amino]propyl}-7-methoxy-4,5-dihydro-2H-benzo[g]indazole-3-carboxylate (1.00 g, 2.02 mmol), and 3-nitrophenyl boronic acid (0.674 g, 4.04 mmol) were slurried in 1-propanol (40 mL). Sodium carbonate (0.428 g, 4.06 mmol) in water (2 mL) was added, followed by palladium acetate (0.0136 g, 0.06 mmol), and triphenyl phosphine (0.0477 g, 0.182 mmol). The reaction mixture was heated to reflux for two hours. (Reaction mixture became homogeneous upon heating). The reaction was cooled to room temperature, and the propanol was evaporated to approximately 5 mL. The slurry was diluted with ethyl acetate (100 mL). Extracted with H₂O (50 mL), and brine (50 mL). The organic phase was dried over MgSO₄, filtered and evaporated. Purified by flash column chromatography in 15% ethyl acetate/85% hexane. The resulting solid was recrystallized from ethyl acetate/hexane. (0.704 g, 65%): ¹H NMR (CDCl₃/300 MHz) 8.47 (s, 1H), 8.17 (d, 1H), 7.90 (d, 1H), 7.81 (s, 1H), 7.56 (s, 1H), 7.56 (t, 1H), 6.89 (s, 1H), 4.96 (s, 1H), 4.63 (t, 2H), 3.93 (s, 3H), 3.87 (s, 3H), 3.12-2.99 (m, 6H), 2.03 (qt, 2H), 1.42 (s, 9H). ESHRMS m/z 537.2378 (M+H, C₂₈H₃₂N₄O₇ requires 537.2344.

Step 2: Preparation of 3-methoxy-2-(3-nitrophenyl)-5,6,8,9,10,11-hexahydro-7H-benzo[g][1,4]diazepino[1,2-b]indazol-7-one.

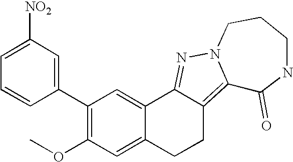

3-Methyl 2-{3-[(tert-butoxycarbonyl)amino]propyl}-7-methoxy-8-(3-nitrophenyl)-4,5-dihydro-2H-benzo[g]indazole-3-carboxylate (0.7 g, 1.3 mmol) was combined with HCl in dioxane (10 mL, of a 4M solution). Stirred at room temperature for two hours. The solvent was then evaporated. A 2:1 mixture of NH₄OH/CH₃OH (20 mL/10 mL respectively) was added and stirred overnight at room temperature. The resulting solid was filtered and washed with methanol. (0.350, 66%): ¹H NMR (CDCl₃/300 MHz) 8.47 (s, 1H), 8.17 (d, 1H), 7.90 (d, 1H), 7.81 (s, 1H), 7.56 (s, 1H), 7.56 (t, 1H), 6.91 (s, 1H), 6.14 (s, 1H), 4.51 (t, 2H), 3.86 (s, 3H), 3.41 (q, 2H), 3.04-2.95 (m, 4H), 2.28 (qt, 2H). ESHRMS m/z 405.1573 (M+H, C₂₂H₂₀N₄O₄ requires 405.1557).

Step 3: Preparatio of 3-hydroxy-2-(3-nitrophenyl)-5,6,8,9,10,11-hexahydro-7H-benzo[g][1,4]diazepino[1,2-b]indazol-7-one trifluoroacetate.

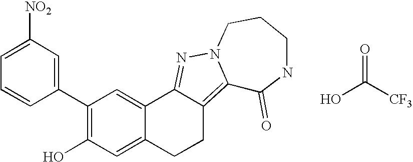

3-methoxy-2-(3-nitrophenyl)-5,6,8,9,10,11-hexahydro-7H-benzo[g][1, 4]diazepino[1,2-b]indazol-7-one (0.300, 0.742 mmol) was slurried in CH₂Cl₂ (10 mL). The mixture was cooled to −20° C. Boron tribromide (7 mL, 7 mmol of a 1.0 M solution in CH₂Cl₂) was added dropwise via syringe. The mixture stirred at room temperature for three hours. Slowly added methanol dropwise to quench the reaction. The solvent was evaporated, and the resulting solid was purified by preparative reverse phase HPLC to yield a white solid (0.170 g): ¹H NMR (DMSO tfa/300 MHz) 8.45 (s, 1H), 8.20-8.17 (m, 2H), 8.07 (d, 1H), 7.75-7.68 (m, 2H), 6.94 (s, 1H), 4.41 (t, 2H), 3.19 (q, 2H), 2.94-2.85 (m, 4H), 2.10 (qt, 2H). ESHRMS m/z 391.1430 (M+H, C₂₁H₁₈N₄O₄ requires 426.1579).

### EXAMPLE 123

This example illustrates the production of 2-(3-aminopropyl)-7-hydroxy-8-(3-nitrophenyl)-2H-benzo[g]indazole-3-carboxylic acid trifluoroacetate.

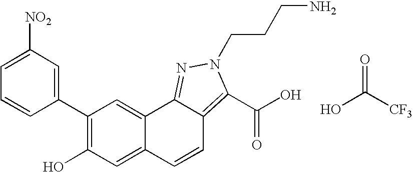

Step 1: Preparation of Methyl 2-{3-[(tert-butoxycarbonyl)amino]propyl}-7-methoxy-8-(3-nitrophenyl)-2H-benzo[g]indazole-3-carboxylate.

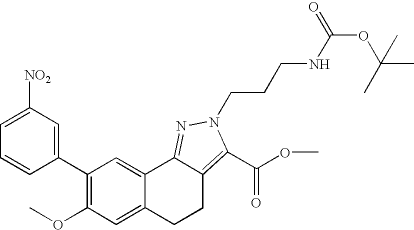

Methyl 2-{3-[(tert-butoxycarbonyl)amino]propyl}-7-methoxy-8-(3-nitrophenyl)-4,5-dihydro-2H-benzo[g]indazole-3-carboxylate (1.00 g, 1.86 mmol) was dissolved in anhydrous dioxane (30 mL). DDQ (0.423 g, 1.86 mmol) was added. Stirred at reflux for 10 hours. Stirred at room temperature overnight. The resulting solid was filtered, and washed with ethyl acetate (50 mL). The mother liquor was extracted with 1.0 N NaOH until the yellow color dissipated. Checked combined aqueous extracts by TLC for any product. Back extracted with ethyl acetate until the entire product was in organic extracts. The combined organic phases were washed with brine, dried over MgSO₄, filtered and evaporated to yield a brown oil. Purified by flash column chromatography with 25% ethyl acetate/75% hexane. Obtained a yellow solid, which was recrystallized from ethanol/water (0.601 g, 61%). ¹H NMR (CDCl₃MHz) 8.57 (s, 1H), 8.55 (s, 1H), 8.24 (d, 1H), 8.03-7.94 (m, 2H), 7.65-7.54 (m, 2H), 7.36 (s, 1H), 4.97 (m, 3H), 4.07 (s, 3H), 3.98 (s, 3H), 3.15 (q, 2H), 2.19 (qt, 2H), 1.41 (s, 9H). ESHRMS m/z 535.2186 (M+H, C₂₂H₂₇N₃O₅ requires 535.2187).

Step 2: Preparation of 2-{3-[(tert-butoxycarbonyl)amino]propyl}-7-methoxy-8-(3-nitrophenyl)-2H-benzo[g]indazole-3-carboxylic Acid.

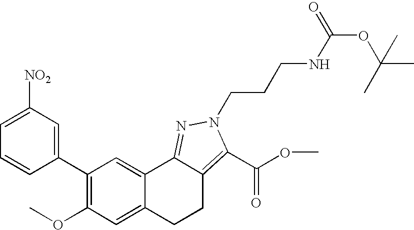

Methyl 2-{3-[(tert-butoxycarbonyl)amino]propyl}-7-methoxy-8-(3-nitrophenyl)-2H-benzo[g]indazole-3-carboxylate (0.304 g, 0.569 mmol) was dissolved in a THF/ethanol/water mixture (10 mL, 7/2/1 respectively). Lithium hydroxide monohydrate (0.048 g, 1.15 mmol was added. Stirred overnight at room temperature. Diluted with ethyl acetate (50 mL) and extracted with 0.1N HCl (2×25 mL), and brine (25 mL). The organic phase was dried over MgSO₄, filtered, and evaporated. The resulting white solid was recrystallized from ethyl acetate/hexane. (0.203 g, 68%): ¹H NMR (DMSO tfa/300 MHz) 8.46 (s, 1H), 8.44 (s, 1H), 8.28 (d, 1H), 8.14 (d, 1H), 7.98 (d, 1H), 7.80 (t, 1H), 7.70-7.60 (m, 2H), 6.88 (s, 1H), 4.87 (t, 2H), 3.98 (s, 3H), 3.01 (q, 2H), 2.03 (qt, 2H), 1.36 (s, 9H). ESHRMS m/z 520.2021 (M+H, C₂₇H₂₈N₄O₇ requires 521.2031).

Step 3: Preparation of 2-(3-aminopropyl)-7-hydroxy-8-(3-nitrophenyl)-2H-benzo[g]indazole-3-carboxylic Acid Trifluoroacetate.

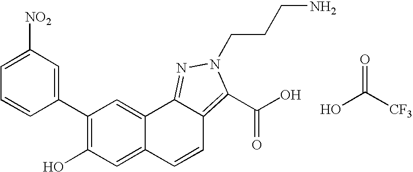

2-f 3-[(tert-butoxycarbonyl)amino]propyl}-7-methoxy-8-(3-nitrophenyl)-2H-benzo[g]indazole-3-carboxylic acid (0.188 g, 0.463 mmol) was slurried in CH₂Cl₂ (10 mL). The mixture was cooled to −20° C. Boron tribromide (5 mL, mmol of a 1.0 M solution in CH₂Cl₂) was added dropwise via syringe. The mixture stirred at room temperature for three hours. Slowly added water dropwise to quench the reaction. The solvent was evaporated, and the resulting solid was purified by preparative reverse phase HPLC to yield a white solid: ¹H NMR (DMSO tfa/300 MHz) 8.54 (s, 1H), 8.44 (s, 1H), 8.28 (d, 1H), 8.19 (d, 1H), 7.92 (d, 1H), 7.81 (t, 1H), 7.74-7.57 (m, 3H), 7.48 (s, 1H), 4.94 (t, 2H), 2.88 (q, 2H), 2.21 (qt, 2H). ESHRMS m/z 407.1384 (M+H, C₂₁H₁₈N₄O₅ requires 407.1350).

### EXAMPLE 124

This example illustrates the production of 3-hydroxy-2-(3-nitrophenyl)-8,9,10,11-tetrahydro-7H-benzo[g][1,4]diazepino[1,2-b]indazol-7-one trifluoroacetate.

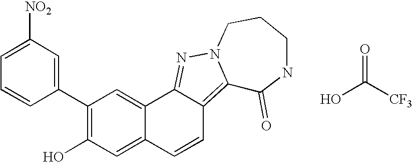

Step 1: Preparation of 3-methoxy-2-(3-nitrophenyl)-8,9,10,11-tetrahydro-7H-benzo[g][1,4]diazepino[1,2-b]indazol-7-one.

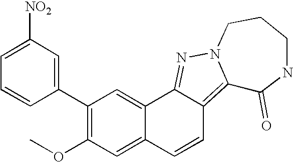

Methyl 2-{3-[(tert-butoxycarbonyl)amino]propyl}-7-methoxy-8-(3-nitrophenyl)-2H-benzo[g]indazole-3-carboxylate (0.270 g, 0.505 mmol) was combined with HCl in dioxane (5 mL, of a 4M solution). Stirred at room temperature for two hours. The solvent was evaporated. A 2:1 mixture of NH₄OH/CH₃OH (6 mL/3 mL respectively) was added. Stirred overnight at room temperature. The resulting solid was filtered and washed with methanol. (0.196, 97%): ¹H NMR (DMSO tfa/300 MHz) 8.46 (s, 1H), 8.42 (s, 1H), 8.26 (d, 1H), 8.13 (d, 1H), 7.91 (d, 1H), 7.78 (t, 1H), 7.66 (s, 1H), 6.60 (d, 1H), 4.80 (t, 2H), 3.97 (s, 3H), 3.28 (q, 2H), 2.31 (qt, 2H). ESHRMS m/z 403.1424 (M+H, C₂₂H₁₈N₄O₄ requires 403.1406).

Step 2: Preparation of 3-hydroxy-2-(3-nitrophenyl)-8,9,10,11-tetrahydro-7H-benzo[g][1,4]diazepino[1,2-b]indazol-7-one trifluoroacetate.

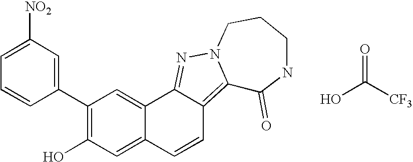

3-Methoxy-2-(3-nitrophenyl)-8,9,10,11-tetrahydro-7H-benzo[g][1,4]-diazepino[1,2-b]indazol-7-one (0.160 g, 0.398 mmol) was slurried in CH₂Cl₂ (10 mL). The mixture was cooled to −20° C. Boron tribromide (4 mL, 4 mmol of a 1.0 M solution in CH₂Cl₂) was added dropwise via syringe. The mixture stirred at room temperature for three hours. Methanol was slowly added dropwise to quench the reaction. The solvent was evaporated, and the resulting solid was purified by preparative reverse phase HPLC to yield a yellow solid: ¹H NMR (DMSO tfa/300 MHz) 8.57 (s, 1H), 8.43 (s, 1H), 8.40 (d, 1H), 8.27-8.20 (m, 2H), 7.82-7.77 (m, 2H), 7.48-7.44(m, 2H), 4.76 (t, 2H), 3.27 (q, 2H), 2.27 (qt, 2H). ESHRMS m/z 389.1255 (M+H, C₂₁H₁₆N₄O₄ requires 389.1250).

### EXAMPLE 125

This example illustrates the production of 2-(2-aminoethyl)-7-hydroxy-8-(3-nitrophenyl)-4,5-dihydro-2H-benzo[g]indazole-3-carboxylic acid trifluoroacetate.

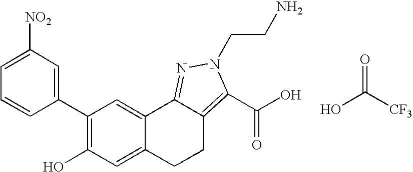

Step 1: Preparation of Methyl-8-bromo-2-{2-[(tert-butoxycarbonyl)amino]ethyl}-7-methoxy-4,5-dihydro-2H-benzo[g]indazole-3-carboxylate.

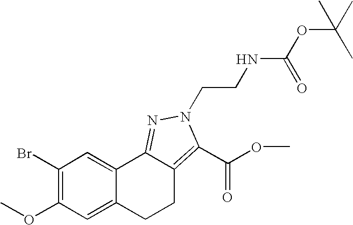

Methyl 8-bromo-7-methoxy-4,5-dihydro-2H-benzo[g]indazole-3-carboxylate (10.0 g, 29.7 mmol) was dissolved in DMF (150 mL). Cooled to 0° C., in a ice-water bath. Lithium t-butoxide (45 mL, 45 mmol of a 1.0 M solution in THF) was added dropwise via addition funnel. Stirred for 2 hours. N-boc-2-bromoethylamine (9.97 g, 44.5 mmol) in DMF (30 mL) was added via addition funnel. The ice bath was removed and the reaction stirred at room temperature overnight. Quenched with NH₄Cl (aq.) (200 mL), and the layers were separated. The aqueous phase was extracted with ethyl acetate (2×200 mL). The combined organic phases were washed with brine, (200 mL), dried over MgSO₄, filtered, and evaporated. The resulting white solid was recrystallized from hot ethanol/water. (11.95 g, 84%): ¹H NMR (CDCl₃/300 MHz) 8.01 (s, 1H), 6.78 (s, 1H), 4.93 (s, 1H), 4.68 (t, 2H), 3.91 (s, 6H), 3.60 (t, 2H), 3.02-2.89 (m, 4H), 1.,41 (s, 9H). ESHRMS m/z 480.1120 (M+H, C₂₁H₂₆BrN₃O₅ requires 480.1134).

Step 2: Preparation of Methyl 2-{2-[(tert-butoxycarbonyl)amino]ethyl}-7-methoxy-8-(3-nitrophenyl)-4,5-dihydro-2H-benzo[g]indazole-3-carboxylate.

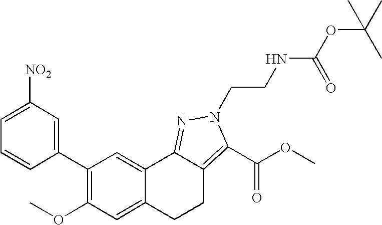

Methyl-8-bromo-2-{2-[(tert-butoxycarbonyl)amino]ethyl}-7-methoxy-4,5-dihydro-2H-benzo[g]indazole-3-carboxylate (4.01 g, 8.35 mmol) and 3-nitrophenyl boronic acid (2.78 g, 16.7 mmol) were slurried in 1-propanol (100 mL). Sodium carbonate (1.77 g, 16.7 mmol) in water (8 mL) was added, followed by palladium acetate (0.056 g, 0.25 mmol), and triphenyl phosphine (0.197 g, 0.750 mmol). The reaction mixture was heated to reflux for four hours. (Reaction mixture became homogeneous upon heating). The reaction cooled to room temperature, and the propanol was evaporated to approximately 10 mL. The slurry was diluted with ethyl acetate (200 mL). Extracted with H₂O (100 mL), and brine (100 mL). The organic phase was dried over MgSO₄, filtered and evaporated. Purified by flash column chromatography in 15% ethyl acetate/85% hexane. The resulting solid was recrystallized from ethyl acetate/hexane. (2.16 g, 50%): ¹H NMR (CDCl₃/300 MHz) 8.46 (s, 1H), 8.17 (d, 1H), 7.90 (s, 1H), 7.82 (s, 1H), 7.56 (t, 1H), 6.90 (s, 1H), 4.94 (s, 1H), 4.69 (t, 2H), 3.93 (s, 3H), 3.87 (s, 3H), 3.61 (t, 2H), 3.07-2.95 (m, 4H), 1.41 (s, 9H). ESHRMS m/z 523.2220 (M+H, C₂₇H₃₀N₄O₇ requires 537.2344).

Step 3: Preparation of 2-{2-[(tert-butoxycarbonyl)amino]ethyl}-7-methoxy-8-(3-nitrophenyl)-4,5-dihydro-2H-benzo[g]indazole-3-carboxylic Acid.

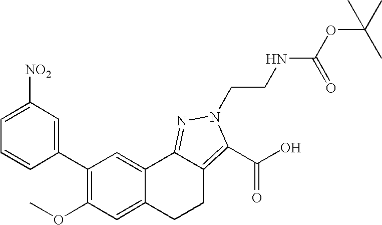

Methyl 2-{2-[(tert-butoxycarbonyl)amino]ethyl}-7-methoxy-8-(3-nitrophenyl)-4,5-dihydro-2H-benzo[g]indazole-3-carboxylate (0.300 g, 0.547 mmol) was dissolved in a THF/ethanol/water mixture (10 mL, 7/2/1 respectively). Lithium hydroxide monohydrate (0.048 g, 1.15 mmol was added. Stirred overnight at room temperature. Diluted with ethyl acetate (50 mL) and extracted with 0.1N HCl (2×25 mL), and brine (25 mL). The organic phase was dried over MgSO₄, filtered, and evaporated. The resulting yellow solid was triturated with hexane. (0.253 g, 87%): ¹H NMR (DMSO tfa/300 MHz) 8.32 (s, 1H), 8.22 (d, 1H), 7.99 (s, 1H), 7.75 (t, 1H), 7.68 (s, 1H), 7.16 (s, 1H), 6.86 (s, 1H), 4.55 (t, 2H), 3.86 (s, 3H), 3.33 (t, 2H), 3.05-2.95 (m, 4H), 1.30 (s, 9H). ESHRMS m/z 509.2071 (M+H, C₂₆H₂₈N₄O₇ requires 509.2031).

Step 4: Preparation of 2-(2-aminoethyl)-7-hydroxy-8-(3-nitrophenyl)-4,5-dihydro-2H-benzo[g]indazole-3-carboxylic Acid Trifluoroacetate.

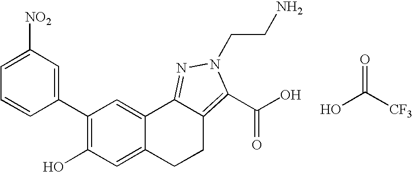

2-{2-[(tert-butoxycarbonyl)amino]ethyl}-7-methoxy-8-(3-nitrophenyl)-4,5-dihydro-2H-benzo[g]indazole-3-carboxylic acid (0.200 g, 0.393 mmol)) was slurried in CH₂Cl₂ (10 mL). The mixture was cooled to −20° C. Boron tribromide (4 mL, 4 mmol of a 1.0 M solution in CH₂Cl₂) was added dropwise via syringe. The mixture stirred at room temperature for three hours. Slowly added water dropwise to quench the reaction. The solvent was evaporated, and the resulting solid was purified by preparative reverse phase HPLC to yield a yellow solid, which was triturated with acetonitrile: ¹H NMR (DMSO tfa/300 MHz) 8.42 (s, 1H), 8.21 (d, 1H), 8.07 (d, 1H), 7.91 (s, 2H), 7.78-7.72 (m, 2H), 6.96 (s, 1H), 4.74 (t, 2H), 3.31 (t, 2H), 2.99-2.90 (m, 4H). ESHRMS m/z 395.1364 (M+H, C₂₀H₁₈N₄O₅ requires 395.1350).

### EXAMPLE 127

This example illustrates the production of 3-hydroxy-2-(3-nitrophenyl)-5,6,9,10-tetrahydrobenzo[g]pyrazino[1,2-b]indazol-7(8H)-one hydrobromide.

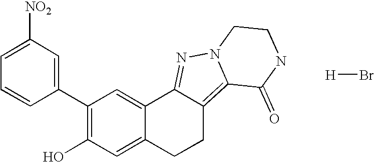

Step 1: Preparation of 3-methoxy-2-(3-nitrophenyl)-5,6,9,10-tetrahydrobenzo[g]pyrazino[1,2-b]indazol-7(8H)-one.

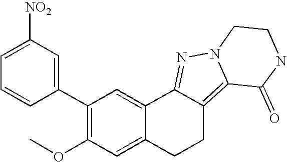

Methyl 2-{2-[(tert-butoxycarbonyl)amino]ethyl}-7-methoxy-8-(3-nitrophenyl)-4,5-dihydro-2H-benzo[g]indazole-3-carboxylate (0.507 g, 0.970 mmol) was combined with HCl in dioxane (6 mL, of a 4M solution). Stirred at room temperature for three hours. The solvent was evaporated. A 2:1 mixture of NH₄OH/CH₃OH (10 mL/5 mL respectively) was added. Stirred overnight at room temperature. The resulting solid was filtered and washed with methanol. (0.330, 87%): ¹H NMR (DMSO tfa/300 MHz) 8.35 (s, 1H), 8.24-8.19(m, 2H), 8.01 (d, 1H), 7.74 (t, 1H)), 7.19 (s, 1H), 4.31 (t, 2H), 3.86 (s, 3H), 3.59(t, 2H), 3.01-2.94 (m, 4H). ESHRMS m/z 391.1424 (M+H, C₂₁H₁₈N₄O₄ requires 391.1401).

Step 2: Preparation of 3-hydroxy-2-(3-nitrophenyl)-5,6,9,10-tetrahydrobenzo[g]pyrazino[1,2-b]indazol-7(8H)-one hydrobromide.

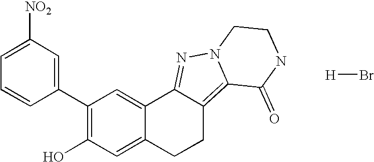

3-Methoxy-2-(3-nitrophenyl)-5,6,9,10-tetrahydro-benzo[g]pyrazino[1,2-b]indazol-7(8H)-one (0.250 g, 0.640 mmol) was slurried in CH₂Cl₂ (5 mL). The mixture was cooled to −20° C. Boron tribromide (2 mL, 2 mmol of a 1.0 M solution in CH₂Cl₂) was added dropwise via syringe. The mixture stirred at room temperature for three hours. Slowly added methanol dropwise to quench the reaction. The solvent was evaporated, and the resulting solid was triturated with acetonitrile: (0.222 g) ¹H NMR (DMSO tfa/300 MHz) 8.45 (s, 1H), 8.21-8.17 (m, 2H), 8.07 (d, 1H), 7.73 (t, 1H), 7.69 (s, 1H), 6.96 (s, 1H), 4.30 (t, 2H), 3.61 (t, 2H), 2.96-2.89 (m, 4H). ESHRMS m/z 377.1276 (M+H, C₂₀H₁₆N₄O₄ requires 377.1244).

### EXAMPLE 128

This example illustrates the production of 2-(2-aminoethyl)-7-hydroxy-8-(3-nitrophenyl)-2H-benzo[g]indazole-3-carboxylic acid trifluoroacetate.

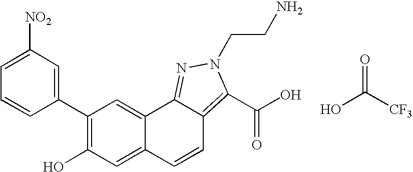

Step 1: Preparation of Methyl 2-{2-[(tert-butoxycarbonyl)amino]ethyl}-7-methoxy-8-(3-nitrophenyl)-2H-benzo[g]indazole-3-carboxylate.

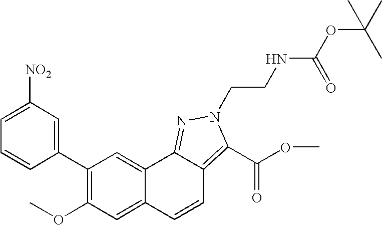

Methyl 2-{2-[(tert-butoxycarbonyl)amino]ethyl}-7-methoxy-8-(3-nitrophenyl)-4,5-dihydro-2H-benzo[g]indazole-3-carboxylate (1.00 g, 1.91 mmol) was dissolved in anhydrous dioxane (30 mL). DDQ (0.652 g, 2.87 mmol) was added. Stirred at reflux overnight. The resulting solid was filtered, and washed with ethyl acetate (50 mL). The mother liquor was extracted with 1.0 N NaOH until the yellow color dissipated. Checked combined aqueous extracts by TLC for any product. Back extracted with ethyl acetate until the entire product was in organic extracts. The combined organic phases were washed with brine, dried over MgSO₄, filtered and evaporated. Purified by flash column chromatography with 20% ethyl acetate/80% hexane. The resulting solid was recrystallized from ethyl acetate/hexane (0.720 g, 72%).

¹H NMR (CDCl3/300 MHz) 8.56 (s, 2H), 8.24 (d, 1H), 8.02-7.94 (m, 2H), 7.64-7.54 (m, 2H), 7.36 (s, 1H), 5.03 (t, 2H), 4.94 (s, 1H), 3.98 (s, 3H), 3.76 (s, 3H), 3.75 (t, 2H), 1.35 (s, 9H). ESHRMS m/z 521.2024 (M+H, C₂₇H₂₈N₄O₇ requires 521.2031).

Step 2: Preparation of 2-{2-[(tert-butoxycarbonyl)amino]ethyl}-7-methoxy-8-(3-nitrophenyl)-2H-benzo[g]indazole-3-carboxylic Acid.

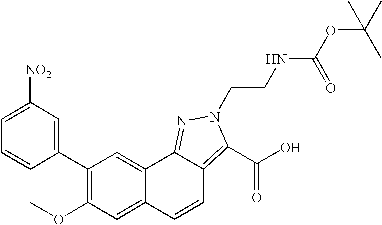

Methyl 2-{2-[(tert-butoxycarbonyl)amino]ethyl}-7-methoxy-8-(3-nitrophenyl)-2H-benzo[g]indazole-3-carboxylate (0.282, 0.542 mmol) was dissolved in a THF/ethanol/water mixture (10 mL, 7/2/1 respectively). Lithium hydroxide monohydrate (0.045 g, 1.08 mmol was added. Stirred overnight at room temperature. Diluted with ethyl acetate (50 mL) and extracted with 0.1N HCl (2×25 mL), and brine (25 mL). The organic phase was dried over MgSO₄, filtered, and evaporated. The resulting white solid was triturated with hexane. (0.225 g, 82%): ¹H NMR (DMSO tfa/300 MHz) 8.44 (s, 2H), 8.29 (d, 1H), 0.812 (d, 1H), 7.98 (d, 1H), 7.80 (t, 1H), 7.69-7.60 (m, 2H), 6.92-(s, 1H); 4:90 (t, 2H), 3.97 (s, 3H), 3.50 (t, 2H), 1.25 (s, 9H). ESHRMS m/z 507.1884 (M+H, C₂₆H₂₆N₄O₇ requires 507.1874).

Step 3: Preparation of 2-(2-aminoethyl)-7-hydroxy-8-(3-nitrophenyl)-2H-benzo[g]indazole-3-carboxylic Acid Trifluoroacetate.

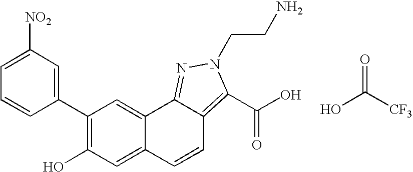

2-{2-[(tert-butoxycarbonyl)amino]ethyl}-7-methoxy-8-(3-nitrophenyl)-2H-benzo[g]indazole-3-carboxylic acid (0.180 g, 0.355 mmol) was slurried in CH₂Cl₂ (5 mL). The mixture was cooled to −20° C. Boron tribromide (2 mL, 2 mmol of a 1.0 M solution in CH₂Cl₂) was added dropwise via syringe. The mixture stirred at room temperature for three hours. Slowly added water dropwise to quench the reaction. The solvent was evaporated, and the resulting solid was purified by preparative reverse phase HPLC to yield a yellow solid triturated with acetonitrile: (0.082 g) ¹H NMR (DMSO tfa/300 MHz) 8.51 (s, 1H), 8.48 (s, 1H), 8.25 (d, 1H), 8.17 (d, 1H), 7.96-7.86(m, 3H), 7.80 (t, 1H), 7.58 (s, 1H), 7.45 (s, 1H), 5.08 (t, 2H), 3.47 (t, 3H). ESHRMS m/z 393.1193 (M+H, C₂₀H₁₆N₄O₅ requires 393.1193).

### EXAMPLE 129

This example illustrates the production of 8-(3-aminophenyl)-2-(3-aminopropyl)-7-hydroxy-4,5-dihydro-2H-benzo[g]indazole-3-carboxylic acid trifluoroacetate.

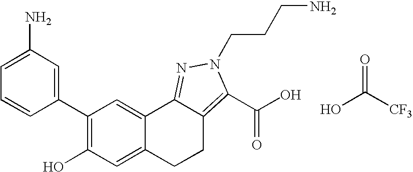

2-(3-aminopropyl)-7-hydroxy-8-(3-nitrophenyl)-4,5-dihydro-2H-benzo[g]indazole-3-carboxylic acid trifluoroacetate (0.115 g) was dissolved in methanol (2 mL). Palladium on carbon (10%)(5 mg) was added. The reaction vessel was sealed and pressurized with H₂ to 40 psi. Heated at 40° C. overnight. The reaction mixture was cooled to room temperature. It was then filtered through celite and evaporated to yield a tan solid, which was triturated with acetonitrile. (0.076 g) ¹H NMR (DMSO tfa/300 MHz) 7.81-7.72 (m, 2H), 7.60-7.48 (m, 4H), 7.28 (s, 1H), 6.93 (s, 1H), 4.58 (t, 2H), 2.97-2.83 (m, 6H), 2.08 (qt, 2H). ESHRMS m/z 379.1749 (M+H, C₂₁H₂₂N₄O₃ requires 379.1765).

### EXAMPLE 130

This example illustrates the production of 2-(3-aminophenyl)-3-hydroxy-8,9,10,1-tetrahydro-7H-benzo[g][1,4]diazepino[1,2-b]indazol-7-one hydrobromide.

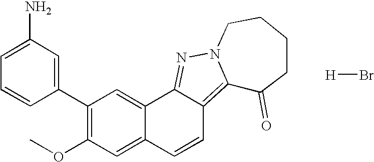

3-methoxy-2-(3-nitrophenyl)-8,9,10,11-tetrahydro-7H-benzo[g][1,4]-diazepino[1,2-b]indazol-7-one (1.13 g, 2.81 mmol) was slurried in CH₂Cl₂ (50 mL). The mixture was cooled to −20° C. Boron tribromide (10 mL, 10 mmol of a 1.0 M solution in CH₂Cl₂) was added dropwise via syringe. The mixture stirred at room temperature for three hours. Slowly added methanol dropwise to quench the reaction. The solvent was evaporated, and the resulting solid was purified by preparative reverse phase HPLC to yield a yellow solid. The solid was dissolved in a THF/methanol mixture (15 mL each). Palladium on carbon (10%)(20 mg) was added. The reaction vessel was sealed and pressurized with H₂ to 40 psi. Heated at 40° C. overnight. The reaction mixture was cooled to room temperature. It was filtered through celite and evaporated to yield a yellow solid. (0.7 g): ¹H NMR (DMSO tfa/300 MHz) 8.36 (s, 1H), 8.29 (s, 1H), 7.77 (d, 1H), 7.42-7.32 (m, 4H), 7.02 (s, 1H), 4.71 (t, 2H), 3.25 (t, 2H), 2.25 (qt, 2H). ESHRMS m/z 359.1534 (M+H, C₂₁H₁₈N₄O₂ requires 359.1503).

### EXAMPLE 131

This example illustrates the production of ethyl 2-(3-aminopropyl)-2,4,5,6-tetrahydropyrazolo[3,4-e]indazole-3-carboxylate.

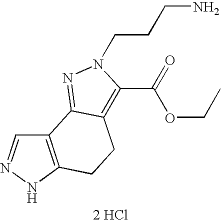

To ethyl 6-trityl-2,4,5,6-tetrahydropyrazolo[3,4-e]indazole-3-carboxylate (5.0 g, 0.01 mol) in DMF (50 mL) at 0° C. was added dropwise lithium t-butoxide (1M in THF, 20 mL). After stirring a half hour at 0° C., added 1-(N-boc-amino)-3-propyl bromide in DMF (10 mL) dropwise. Contents were stirred overnight and partitioned between EtOAc and water. The EtOAc layer was washed with brine, dried over MgSO₄ and concentrated in vacuo leaving an amber oil, 8.9 g. The oil was purified by silica gel chromatography, eluting with 15% EtOAc/hexanes to give ethyl 2-{3-[(tert-butoxycarbonyl)-amino]propyl}-7-trityl-2,4,5,6-tetrahydropyrazolo[3,4-e]indazole-3-carboxylate as a white foam, 2.2 g. The white foam was mixed with 4N HCl in dioxane (20 mL). A white solid was filtered to give the desired product, 2.0 g (61% yield). FABHRMS m/z 290.1617 (M+H, C₁₄H₂₀N₅O₂ requires 290.1612). ¹H NMR (DMSO-d₆+TFA/300 MHz): 8.02 (s, 1H); 7.80 (br s, 3H); 4.55 (t, 2H); 4.37 (q, 2H); 3.10-2.90 (m, 4H); 2.90-2.75 (m, 2H); 2.10-2.00 (m, 2H); 1.38 (t, 3H).

Anal. Calcd for C₁₄H₁₉N₅O₂ (0.4H₂O, 2HCl): C, 45.51; H, 5.95; N, 18.96. Found: C, 45.64; H, 5.89; N, 18.77.

### EXAMPLE 132

This example illustrates the production of ethyl 2-(3-aminopropyl)-2,4,5,6-tetrahydropyrazolo[3,4-e]indazole-3-carboxylate.

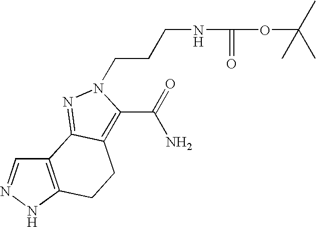

The protected ester from ethyl 2-(3-aminopropyl)-2,4,5,6-tetrahydropyrazolo[3,4-e]indazole-3-carboxylate dihydrochloride was reacted according to the procedure for the production of 8-hydroxy-4,5-dihydro-2H-benzo[g]indazole-3-carboxamide to give the desired as a light amber solid (75% yield). FABHRMS m/z 361.2012 (M+H, C₁₇H₂₅N₆O₃ requires 361.1983).

Anal. Calcd for C₁₇H₂₄N₆O₃ (0.1H₂O): C, 56.37; H. 6.73; N, 23.20. Found: C, 56.03; H, 6.87; N, 22.98.

### EXAMPLE 133

This example illustrates the production of 2-(3-aminopropyl)-2,4,5,6-tetrahydropyrazolo[3,4-e]indazole-3-carboxylic acid dihydrochloride.

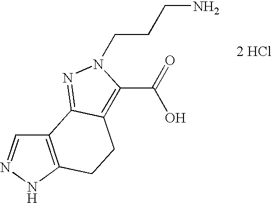

The protected ester from ethyl 2-(3-aminopropyl)-2,4,5,6-tetrahydropyrazolo[3,4-e]indazole-3-carboxylate dihydrochloride was reacted according to the procedure for the production of 7-hydroxy-4,5-dihydro-2H-benzo[g]indazole-3-carboxylic acid to give a white solid which is 2-{3-[(tert-butoxy-carbonyl)amino]propyl}-2,4,5,6-tetrahydropyrazolo[3,4-e]indazole-3-carboxylic acid. The solid was triturated with 4N HCl in dioxane, stirred overnight and filtered to give the desired product as a white solid (61% yield). FABHRMS m/z 262.1284 (M+H, C₁₂H₁₆N₅O₂ requires 262.1299). ¹H NMR (DMSO-d₆+TFA/300 MHz): 8.20 (s, 1H); 7.95 (br s, 3H); 4.58 (t, 2H); 3.10-2.90 (m, 4H); 2.85-2.75 (m, 2H); 2.20-2.00 (m, 2H).

Anal. Calcd for C₁₂H₁₅N₅O₂ (2.5 HCl): C, 40.90; H, 5.00; N, 19.87. Found: C, 40.78; H, 5.15; N, 18.96.

### EXAMPLE 134

This example illustrates the production of 2-(3-aminopropyl)-2,4,5,6-tetrahydro-pyrazolo[3,4-e]indazole-3-carboxamide dihydrochloride.

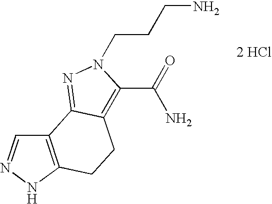

ethyl 2-(3-aminopropyl)-2,4,5,6-tetrahydropyrazolo[3,4-e]indazole-3-carboxylate dihydrochloride was reacted according to the procedure for the production of 8-hydroxy-4,5-dihydro-2H-benzo[g]indazole-3-carboxamide to give a white solid (52% yield). FABHRMS m/z 261.1498 (M+H, C₁₂H₁₇N₆O requires 261.1464).

Anal. Calcd for C₁₂H₁₆N₆O (2.4 HCl): C, 41.44; H, 5.33; N, 24.16. Found: C, 41.84; H, 5.08; N, 23.87.

### EXAMPLE 135

This example illustrates the production of ethyl 1-{3-[(tert-butoxycarbonyl)amino]propyl}-7-trityl-1,4,5,6-tetrahydropyrazolo[3,4-e]indazole-3-carboxylate.

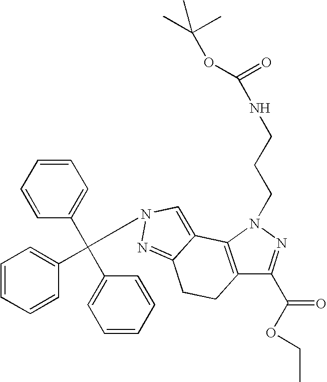

Ethyl 1-{3-[(tert-butoxycarbonyl)amino]propyl}-6-trityl-1,4,5,6-tetrahydropyrazolo[3,4-e]indazole-3-carboxylate was prepared according to the procedure for the production of ethyl 2-(3-aminopropyl)-2,4,5,6-tetrahydropyrazolo[3,4-e]indazole-3-carboxylate dihydrochloride, using THF as the solvent, to give the desired product after silica gel chromatography, eluting with 25% EtOAc/hexanes, as a white solid (30% yield). FABHRMS m/z 632.3209 (M+H, C₃₈H₄₂N₅O₄ requires 632.3237). ¹H NMR (CDCl₃/300 MHz): 7.40-7.20 (m, 16H); 4.72 (br, 1H); 4.43 (q, 2H); 4.20 (t, 2H); 3.22-3.15 (m, 2H); 3.10-2.95 (m, 4H); 1.90 (quintet, 2H); 1.45 (t, 3H); 1.40 (s, 9H).

Anal. Calcd for C₃₈H₄₁N₅O₄: C, 72.24; H, 6.54; N, 11.09. Found: C, 72.22; H, 6.72; N, 10.66.

### EXAMPLE 136

This example illustrates the production of 1-(3-aminopropyl)-1,4,5,6-tetrahydropyrazolo[3,4-e]indazole-3-carboxamide dihydrochloride.

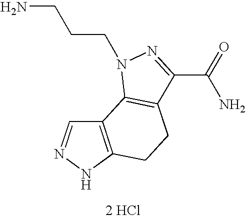

Ethyl 1-{3-[(tert-butoxycarbonyl)amino]propyl}-6-trityl-1,4,5,6-tetrahydropyrazolo[3,4-e]indazole-3-carboxylate, was reacted according to the procedure for the production of 8-hydroxy-4,5-dihydro-2H-benzo[g]indazole-3-carboxamide to give the desired product as a white solid after trituration with 4N HCl in dioxane (70% yield). FABHRMS m/z 261.1434 (M+H, C₁₂H₁₇N₆O requires 261.1458). ¹H NMR (DMSO-d₆+5%TFA/300 MHz): 8.30 (s, 1H); 8.10 (br, 3H); 4.40 (t, 2H); 3.10-3.00 (m, 2H); 3.00-2.80 (m, 4H); 2.20-2.05 (m, 2H).

Anal. Calcd for C₁₂H₁₆N₆O (3 HCl): C, 37.18; —H, 5.46; N, 21.68. Found: C, 37.31; H, 5.88; N, 24.88.

### EXAMPLE 137

This example illustrates the production of ethyl 1-(2,2,2-trifluoroethyl)-7-trityl-1,4,5,6-tetrahydropyrazolo[3,4-e]indazole-3-carboxylate.

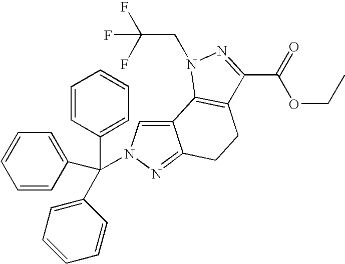

Ethyl oxo(4-oxo-1-trityl-4,5,6,7-tetrahydro-1H-indazol-5-yl)acetate- or ethyl oxo(4-oxo-2-trityl-4,5,6,7-tetrahydro-2H-indazol-5-yl)acetate or ethyl oxo(4-oxo-2-trityl-4,5,6,7-tetrahydro-2H-indazol-5-yl)acetate (12.0 g, 0.025 mol) in glacial acetic acid (100 mL) was added dropwise trifluoroethylhydrazine (70% aqueous, 5.0 g, 0.03 mol). Contents were stirred overnight and diluted with water (100 mL), precipitating a solid, 16.6 g. The solid was triturated with 25% EtOAc/hexanes and filtered to give the desired as a white solid, 5.9 g (42% yield). FABHRMS m/z 557.2186 (M+H, C₃₂H₂₈F₃N₄O₂ requires 557.2159). ¹H NMR (CDCl₃/300 MHz): 7.40-7.15 (m, 15H); 4.70 (q, 2H); 4.44 (q, 2H); 3.20 (t, 2H); 3.00 (t, 2H); 1.45 (t, 3H).

Anal. Calcd for C₃₂H₂₇F₃N₄O₂ (0.8H₂O): C, 67.31; H, 5.05; N, 9.81. Found: C, 67.26; H, 4.93; N, 9.03.

### EXAMPLE 138

This example illustrates the production of ethyl 2-(2,2,2-trifluoroethyl)-2,4,5,6-tetrahydropyrazolo[34-e]indazole-3-carboxylate hydrochloride.

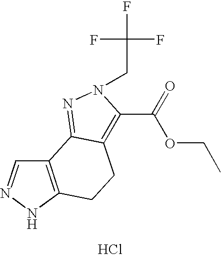

The material obtained in the 25% EtOAc/hexanes filtrate of ethyl 1-(2,2,2-trifluoroethyl)-6-trityl-1,4,5,6-tetrahydropyrazolo[3,4-e]indazole-3-carboxylate by concentration in vacuo was a waxy yellow solid, 3.0 g. The waxy solid was purified by silica gel chromatography, eluting with 10% EtOAc/hexanes to give a white foam, 430 mg. The foam was mixed with 4N HCl in dioxane (5 mL), stirred one hour, diluted with EtOAc and filtered to give the desired product as a white solid, 350 mg (4% yield). FABHRMS m/z 315.1077 (M+H, C₁₃H₁₄F₃N₄O₂ requires 315.1063).

Anal. Calcd for C₁₃H₁₃F₃N₄O₂ (0.9 HCl): C, 44.99; H, 4.04; N, 16.14. Found: C, 45.17; H, 3.86; N, 15.96.

### EXAMPLE 139

This example illustrates the production of ethyl 1-(2,2,2-trifluoroethyl)-1,4,5,6-tetrahydropyrazolo[3,4-e]indazole-3-carboxylate hydrochloride.

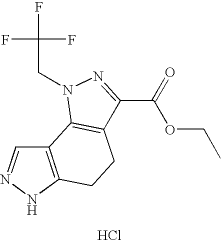

Ethyl 1-(2,2,2-trifluoroethyl)-6-trityl-1,4,5,6-tetrahydropyrazolo[3,4-e]indazole-3-carboxylate (442 mg, 0.8 mmol) was mixed with 4N HCl in dioxane, stirred one hour, diluted with EtOAc and filtered to give the desired product as a white solid, 192 mg (77% yield). FABHRMS m/z 315.1088 (M+H, C₁₃H₁₄F₃N₄O₂ requires 315.1063.

Anal. Calcd for C₁₃H₁₃F₃N₄O₂ (HCl): C, 44.52; H, 4.02; N, 15.97. Found: C, 44.53; H, 3.93; N, 15.96.

### EXAMPLE 140

This example illustrates the production of 1-(2,2,2-trifluoroethyl)-7-trityl-1,4,5,6-tetrahydropyrazolo[3,4-e]indazole-3-carboxylic acid.

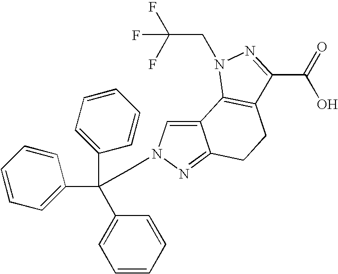

Ethyl 1-(2,2,2-trifluoroethyl)-6-trityl-1,4,5,6-tetrahydropyrazolo[3,4-e]indazole-3-carboxylate was reacted according to the procedure for the production of 7-hydroxy-4,5-dihydro-2H-benzo[g]indazole-3-carboxylic acid to give the desired as a white solid (88% yield). FABHRMS m/z 529.1875 (M+H, C₃₀H₂₃F₃N₄O₂ requires 529.1846). ¹H NMR (DMSO-d₆/300 MHz): 7.85 (s, 1H); 7.42-7.10 (m, 15H); 5.23 (q, 2H); 3.10-2.90 (m, 4H).

Anal. Calcd for C₃₀H₂₃F₃N₄O₂ (0.5H₂O): C, 67.03; H, 4.50; N, 10.42. Found: C, 67.23; H, 4.24; N, 9.92.

### EXAMPLE 141

This example illustrates the production of 1-(2,2,2-trifluoroethyl)-1,4,5,6-tetrahydropyrazolo[3,4-e]indazole-3-carboxylic acid hydrochloride.

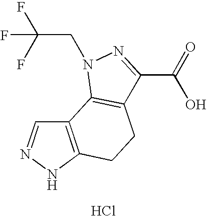

1-(2,2,2-trifluoroethyl)-6-trityl-1,4,5,6-tetrahydropyrazolo[3,4-e]indazole-3-carboxylic acid (1.6 g, 3 mmol) and 4N HCl in dioxane (25 mL) were stirred overnight. Contents were filtered to give the desired product as a white solid, 831 mg (86% yield). FABHRMS m/z 287.0728 (M+H, C₁₁H₁₀F₃N₄O₂ requires 287.0750).

Anal. Calcd for C₁₁H₉F₃N₄O₂ (HCl, 0.2H₂O): C, 40.49; H, 3.21; N, 17.17. Found: C, 40.50; H, 3.40; N, 16.62.

### EXAMPLE 143

This example illustrates the production of 2-(2,2,2-trifluoroethyl)-2,4,5,6-tetrahydropyrazolo[3,4-e]indazole-3-carboxylic acid.

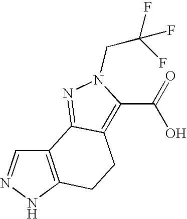

Ethyl 2-(2,2,2-trifluoroethyl)-2,4,5,6-tetrahydropyrazolo[3,4-e]indazole-3-carboxylate hydrochloride was reacted according to the procedure for the production of 7-hydroxy-4,5-dihydro-2H-benzo[g]indazole-3-carboxylic acid to give the desired product as a white solid (93% yield). FABHRMS m/z 287.0745 (M+H, C₁₁H₁₀F₃N₄O₂ requires 287.0750).

Anal. Calcd for C₁₁H₉F₃N₄O₂ (0.4H₂O): C, 45.03; H, 3.37; N, 19.09. Found: C, 45.29; H, 3.00; N, 18.83.

### EXAMPLE 144

This example illustrates the production of ethyl 1-(2-hydroxyethyl)-7-trityl-1,4,5,6-tetrahydropyrazolo[3,4-e]indazole-3-carboxylate.

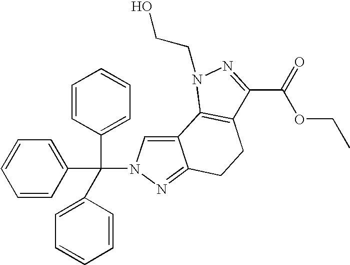

To 2-hydroxyethylhydrazine (8.5 g, 0.1 mol) in methanol (300 mL) at 0° C. was added drop wise ethyl oxo(4-oxo-1-trityl-4,5,6,7-tetrahydro-1H-indazol-5-yl)acetate- or ethyl oxo(4-oxo-2-trityl-4,5,6,7-tetra hydro-2H-indazol-5-yl)acetate or ethyl oxo(4-oxo-2-trityl-4,5,6,7-tetrahydro-2H-indazol-5-yl)acetate (38.2 g, 0.08 mol) in CH₂Cl₂/methanol (1:1,100 mL). Contents were stirred overnight and a solid was filtered to give the desired product as a white solid, 6.6 g. The filtrate was concentrated by half and more solid was filtered, 8.5 g, also desired product (36% yield). FABHRMS m/z 519.2380 (M+H, C₃₂H₃₁N₄O₃ requires 519.2396). ¹H NMR (DMSO-d₆+TFA/300 MHz): 7.77 (s, 1H); 7.40-7.10 (m, 15H); 4.23 (q, 2H); 4.13-4.10 (m, 2H); 3.70-3.60 (m, 2H); 3.05-2.95 (m, 2H); 2.82-2.75 (m, 2H); 1.30 (t, 3H).

Anal. Calcd for C₃₂H₃₀N₄O₃: C, 74.11; H, 5.83; N, 10.80. Found: C, 73.84; H, 5.91; N, 10.85.

### EXAMPLE 145

This example illustrates the production of ethyl 1-(2-hydroxyethyl)-1,4,5,6-tetrahydropyrazolo[3,4-e]indazole-3-carboxylate hydrochloride.

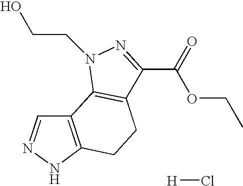

Ethyl 1-(2-hydroxyethyl)-6-trityl-1,4,5,6-tetrahydropyrazolo[3,4-e]indazole-3-carboxylate (3.0 g, 0.006 mol) and 4N HCl in dioxane (20 mL) were stirred overnight and a solid was filtered to give the desired product as a white solid, 1.13 g (71% yield). FABHRMS m/z 277.1290 (M+H, C₁₃H₁₇N₄O₃ requires 277.1295).

Anal. Calcd for C₁₃H₁₇N₄O₃ (1.2 HCl): C, 48.79; H, 5.42; N, 17.51. Found: C, 48.47; H, 5.83; N, 17.56.

### EXAMPLE 146

This example illustrates the production of 1-(2-hydroxyethyl)-1,4,5,6-tetrahydropyrazolo[3,4-e]indazole-3-carboxylic acid.

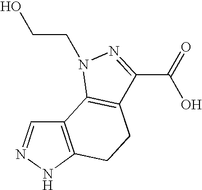

Ethyl 1-(2-hydroxyethyl)-1,4,5,6-tetrahydropyrazolo[3,4-e]indazole-3-carboxylate hydrochloride was reacted according to the procedure for the production of 7-hydroxy-4,5-dihydro-2H-benzo[g]indazole-3-carboxylic acid (76% yield). FABHRMS m/z 249.1021 (M+H, C₁₁H₁₃N₄O₃ requires 249.0982).

Anal. Calcd for C₁₁H₁₂N₄O₃: C, 52.91; H, 4.77; N, 22.90. Found: C, 53.22; H, 4.87; N, 22.57

### EXAMPLE 147

This example illustrates the production of 1-(2-hydroxyethyl)-1,4,5,6-tetrahydropyrazolo[3,4-e]indazole-3-carboxamide.

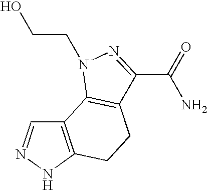

Ethyl 1-(2-hydroxyethyl)-6-trityl-1,4,5,6-tetrahydropyrazolo[3,4-e]indazole-3-carboxylate and 4N HCl in dioxane were stirred overnight and filtered to give ethyl 1-(2-hydroxyethyl)-1,4,5,6-tetrahydropyrazolo[3,4-e]indazole-3-carboxylate as a white solid. The white solid was reacted according to the procedure for the production of 8-hydroxy-4,5-dihydro-2H-benzo[g]indazole-3-carboxamide to give the desired product as a white solid (59% yield).

FABHRMS m/z 248.1151 (M+H, C₁₁H₁₄N₅O₂ requires 248.1147). ¹H NMR (DMSO-d₆+TFA/300 MHz): 8.10 (s, 1H); 4.25 (t, 2H); 3.80 (t, 2H); 3.02 (t, 2H); 2.80 (t, 2H).

Anal. Calcd for C₁₁H₁₃N₅O₂: C, 53.43; H, 5.30; N, 28.32. Found: C, 53.98; H, 5.30; N, 27.99.

### EXAMPLE 148

This example illustrates the production of ethyl 1-benzyl-1,4,5,6-tetrahydropyrazolo[3,4-e]indazole-3-carboxylate.

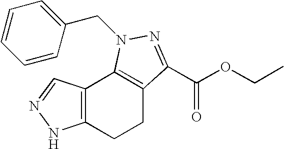

Ethyl oxo(4-oxo-1-trityl-4,5,6,7-tetrahydro-1H-indazol-5-yl)acetate- or ethyl oxo(4-oxo-2-trityl-4,5,6,7-tetrahydro-2H-indazol-5-yl)acetate or ethyl oxo(4-oxo-2-trityl-4,5,6,7-tetrahydro-2H-indazol-5-yl)acetate and benzylhydrazine dihydrochloride were reacted according to the procedure for the production of ethyl 8-hydroxy-4,5-dihydro-2H-benzo[g]indazole-3-carboxylate to give the desired product as a white solid (46% yield). FABHRMS m/z 323.1504 (M+H, C₁₈H₁₉N₄O₂ requires 323.1503).

Anal. Calcd for C₁₈H₁₈N₄O₂: C, 67.07; H, 5.63; N, 17.38. Found: C, 66.82; H, 5.48; N, 17.34.

### EXAMPLE 149

This example illustrates the production of 1-benzyl-1,4,5,6-tetrahydropyrazolo[3,4-e]indazole-3-carboxamide.

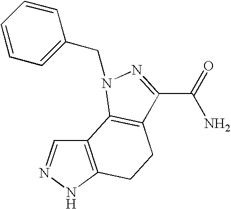

Ethyl 1-benzyl-1,4,5,6-tetrahydropyrazolo[3,4-e]indazole-3-carboxylate was reacted according to the procedure for the production of 8-hydroxy-4,5-dihydro-2H-benzo[g]indazole-3-carboxamide to give the desired product as a tan solid (63% yield). FABHRMS m/z 294.1372 (M+H, C₁₆H₁₆N₅O requires 294.1349).

Anal. Calcd for C₁₆H₁₅N₅O: C, 65.52; H, 5.15; N, 23.88. Found: C, 65.27; H, 4.95; N, 23.85.

### EXAMPLE 150

This example illustrates the production of ethyl 7-trityl-2,4,5,6-tetrahydropyrazolo[3,4-e]indazole-3-carboxylate.

Ethyl oxo(4-oxo-1-trityl-4,5,6,7-tetrahydro-1H-indazol-5-yl)acetate- or ethyl oxo(4-oxo-2-trityl-4,5,6,7-tetrahydro-2H-indazol-5-yl)acetate or ethyl oxo(4-oxo-2-trityl-4,5,6,7-tetrahydro-2H-indazol-5-yl)acetate was reacted according to the procedure for the production of ethyl 8-hydroxy-4,5-dihydro-2H-benzo[g]indazole-3-carboxylate to give the desired product as a white solid (83% yield). ¹H NMR (CDCl₃/300 MHz): 7.65 (s, 1H); 7.40-7.20 (m, 15H); 4.41 (q, 2H); 3.20-3.10 (m, 2H); 3.05-2.95 (m, 2H); 1.43 (t, 3H).

Anal. Calcd for C₃₀H₂₆N₄O₃(0.8H₂O): C, 73.69; H, 5.69; N, 11.46. Found: C, 73.59; H, 6.02; N, 11.03.

### EXAMPLE 151

This example illustrates the production of ethyl 2-{2-[(tert-butoxycarbonyl)amino]ethyl}-7-trityl-2,4,5,6-tetrahydropyrazolo[3,4-e]indazole-3-carboxylate.

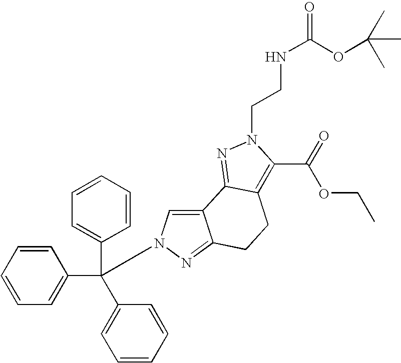

Ethyl 6-trityl-2,4,5,6-tetrahydropyrazolo[3,4-e]indazole-3-carboxylate and 2-(N-Boc-aminoethyl) bromide were reacted according to the procedure for the production of ethyl 2-(3-aminopropyl)-2,4,5,6-tetrahydropyrazolo[3,4-e]indazole-3-carboxylate dihydrochloride to give the desired product as a white solid (60% yield). FABHRMS m/z 618.3076 (M+H, C₃₇H₄₀N₅O₄ requires 618.3080). ¹H NMR (CDCl₃/300 MHz): 7.63 (s, 1H); 7.40-7.20 (m, 15H); 4.90 (br s, 1H); 4.69-4.60 (m, 2H); 4.40 (q, 2H); 3.60-3.55 (m, 2H); 3.20-3.10 (m, 2H); 3.00-2.90 (m, 2H); 1.42 (t, 3H); 1.40 (s, 9H).

Anal. Calcd for C₃₇H₃₉N₅O₄: C, 71.94; H, 6.36; N, 11.34. Found: C, 71.65; H, 6.07; N, 11.32.

### EXAMPLE 152

This example illustrates the production of 2-{2-[(tert-butoxycarbonyl)amino]ethyl}-7-trityl-2,4,5,6-tetrahydropyrazolo[3,4-e]indazole-3-carboxylic acid.

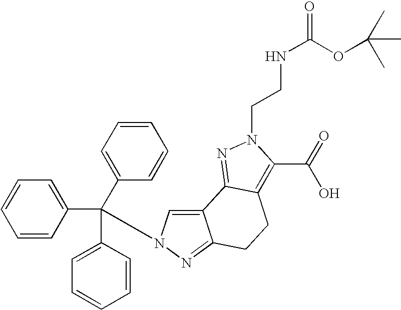

Ethyl 2-{2-[(tert-butoxycarbonyl)amino]ethyl}-6-trityl-2,4,5,6-tetrahydropyrazolo[3,4-e]indazole-3-carboxylate was reacted according to the procedure for the production of 7-hydroxy-4,5-dihydro-2H-benzo[g]indazole-3-carboxylic acid to give the desired product as a white solid (90% yield). FABHRMS m/z 590.2748 (M+H, C₃₅H₃₆N₅O₄ requires 590.2767). ¹H NMR (DMSO-d₆+5%TFA/300 MHz): 7.45-7.30 (m, 10H); 7.20-7.05 (m, 6H); 6.78 (br s, 1H); 4.50-4.40 (m, 2H); 3.35-3.25 (m, 2H); 3.10-3.00 (m, 2H); 2.90-2.80 (m, 2H); 1.14 (s, 9H).

Anal. Calcd for C₃₅H₃₅N₅O₄ (1.0H₂O): C, 67.18; H, 6.28; N, 11.19. Found: C, 66.80; H, 5.93; N. 11.06.

### EXAMPLE 153

This example illustrates the production of 2-(2-aminoethyl)-2,4,5,6-tetrahydropyrazolo [3,4-e]indazole-3-carboxylic acid trihydrochloride:

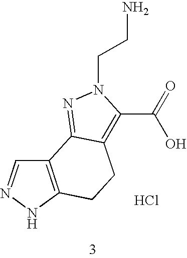

2-{2-[(tert-butoxycarbonyl)amino]ethyl}-6-trityl-2,4,5,6-tetrahydropyrazolo[3,4-e]indazole-3-carboxylic acid (442 mg, 0.75 mmol) and 4N HCl in dioxane (10 mL) were stirred overnight. Contents were filtered to give the desired product as a white solid, 140 mg (58% yield). FABHRMS m/z 248.1100 (M+H, C₁₁H₁₄N₅O₂ requires 248.1147). ¹H NMR (DMSO-d₆/300 MHz): 8.40 (br s, 3H); 8.05 (s, 1H); 4.80-4.70 (m, 2H); 3.30-3.20 (m, 2H); 3.10-3.00 (m, 2H); 2.95-2.85 (m, 2H).

Anal. Calcd for C₁₁H₁₃N₅O₂ (3 HCl, 1.0H₂O): C, 35.26; H, 4.84; N, 18.69. Found: C, 35.40; H, 4.45; N, 18.81.

### EXAMPLE 154

This example illustrates the production of ethyl 2-(2-hydroxyethyl)-7-trityl-2,4,5,6-tetrahydropyrazolo[3,4-e]indazole-3-carboxylate.

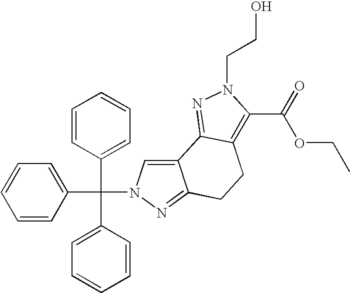

To 2-hydroxyethylhydrazine (8.5 g, 0.1 mol) in methanol (300 mL) at 0° C. was added dropwise ethyl oxo(4-oxo-1-trityl-4,5,6,7-tetrahydro-1H-indazol-5-yl)acetate- or ethyl oxo(4-oxo-2-trityl-4,5,6,7-tetrahydro-2H-indazol-5-yl)acetate or ethyl oxo(4-oxo-2-trityl-4,5,6,7-tetrahydro-2H-indazol-5-yl)acetate (38.2 g, 0.08 mol) in CH₂Cl₂/methanol (1:1,100 mL). Contents were stirred overnight and a solid was filtered to give ethyl 1-(2-hydroxyethyl)-6-trityl-1,4,5,6-tetrahydropyrazolo[3,4-e]indazole-3-carboxylate as a white solid, 6.6 g. The filtrate was concentrated by half and more solid was filtered, 8.5 g, also ethyl 1-(2-hydroxyethyl)-6-trityl-1,4,5,6-tetrahydropyrazolo[3,4-e]indazole-3-carboxylate. The filtrate was purified by silica gel chromatography eluting with 40% EtOAc/hexanes to give the desired product as a white solid, 2.6 g (6% yield). FABHRMS m/z 519.2422 (M+H, C₃₂H₃₁N₄O₃ requires 519.2396).

Anal. Calcd for C₃₂H₃₀N₄O₃ (0.3H₂O): C, 73.35; H, 5.89; N, 10.69. Found: C, 73.01; H, 5.52; N, 10.37.

### EXAMPLE 155

This example illustrates the production of 2-(2-hydroxyethyl)-2,4,5,6-tetrahydropyrazolo[3,4-e]indazole-3-carboxylic acid.

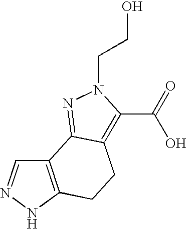

Ethyl 2-(2-hydroxyethyl)-6-trityl-2,4,5,6-tetrahydropyrazolo[3,4-e]indazole-3-carboxylate was reacted according to the procedure for the production of 7-hydroxy-4,5-dihydro-2H-benzo[g]indazole-3-carboxylic acid to give the intermediate carboxylic acid with the trityl group still intact. The intermediate was stirred with 4N HCl in dioxane and stirred overnight, diluted with EtOAc and filtered to give the desired product as a white solid (87% yield).

FABHRMS m/z 249.0975 (M+H, C₁₁H₁₃N₄O₃ requires 249.0982).

Anal. Calcd for C₁₁H₁₂N₄O₃(2 HCl): C, 41.14; H, 4.39; N, 17.44. Found: C, 41.03; H, 4.56; N, 17.39.

### EXAMPLE 156

This example illustrates the production of 4,5,8,9-tetrahydro-3H-pyrazino[1,2-b]pyrazolo[3,4-g]indazol-6(7H)-one.

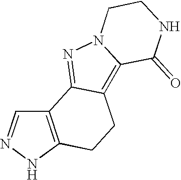

Ethyl 2-{2-[(tert-butoxycarbonyl)amino]ethyl}-6-trityl-2,4,5,6-tetrahydropyrazolo-[3,4-e]indazole-3-carboxylate (1.3 g, 0.002 mol) and 4N HCl in dioxane (20 mL) were stirred overnight and filtered to give a white solid, 630 mg, analysis of which shows deprotection of both the Boc and trityl groups. This solid was reacted according to the procedure for the production of 8-hydroxy-4,5-dihydro-2H-benzo[g]indazole-3-carboxamide to give the desired product as a white solid, 377 mg (82% yield). FABHRMS m/z 230.1033 (M+H, C₁₁H₁₂N₅O requires 230.1036). ¹H NMR (DMSO-d₆+5%TFA/300 MHz): 8.15 (br s, 1H); 8.00 (s, 1H); 4.22 (t, 2H); 3.60 (t, 2H); 3.07-2.83 (m, 4H).

Anal. Calcd for C₁₁H₁₁N₅O (0.2H₂O): C, 56.74; H, 4.93; N, 30.08. Found: C, 56.92; H, 4.61; N, 29.95.

### EXAMPLE 157.

This example illustrates the production of ethyl 2-[3-(tetrahydro-2H-pyran-2-yloxy)propyl]-7-trityl-2,4,5,6-tetrahydropyrazolo[3,4-e]indazole-3-carboxylate.

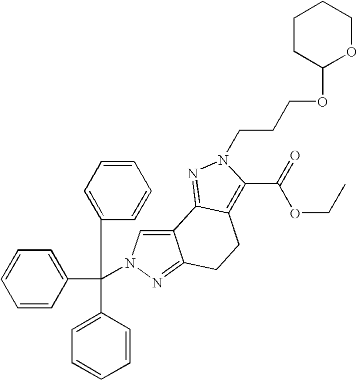

Ethyl 6-trityl-2,4,5,6-tetrahydropyrazolo[3,4-e]indazole-3-carboxylate and 2-(3-bromopropoxy)tetrahydro-2H-pyran were reacted according to the procedure for the production of ethyl 2-(3-aminopropyl)-2,4,5,6-tetrahydropyrazolo[3,4-e]indazole-3-carboxylate dihydrochloride to give the desired product as white crystals, 2.0 g (26% yield). FABHRMS m/z 617.3163 (M+H, C₃₈H₄₁N₄O₄ requires 617.3122).

Anal. Calcd for C₃₈H₄₀N₄O₄: C, 74.00; H, 6.54; N, 9.08. Found: C, 73.60; H, 6.58; N, 9.02.

### EXAMPLE 158

This example illustrates the production of ethyl 2-(3-hydroxypropyl)-2,4,5,6-tetrahydropyrazolo[3,4-e]indazole-3-carboxylate hydrochloride.

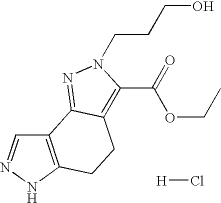

Ethyl 2-[3-(tetrahydro-2H-pyran-2-yloxy)propyl]-6-trityl-2,4,5,6-tetrahydropyrazolo[3,4-e]indazole-3-carboxylate (1.8 g, 0.003 mol) and 4N HCl in dioxane were stirred overnight and a solid was filtered to give the desired product as a white solid, 933 mg (98% yield). FABHRMS m/z 291.1444 (M+H, C₁₄H₁₉N₄O₃ requires 291.1452).

Anal. Calcd for C₁₄H₁₈N₄O₃(0.6 HCl): C, 53.86; H, 6.01; N, 17.95. Found: C, 53.74; H, 5.89; N, 18.11.

### EXAMPLE 159

This example illustrates the production of 2-(3-hydroxypropyl)-2,4,5,6-tetrahydropyrazolo[3,4-e]indazole-3-carboxylic acid.

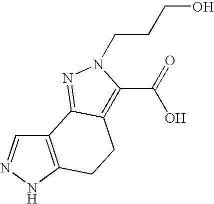

2-(3-hydroxypropyl)-2,4,5,6-tetrahydropyrazolo[3,4-e]indazole-3-carboxylic acid was prepared according to the procedure for the production of 8-hydroxy-4,5-dihydro-2H-benzo[g]indazole-3-carboxamide, using ethyl 2-(3-hydroxypropyl)-2,4,5,6-tetrahydropyrazolo[3,4-e]indazole-3-carboxylate hydrochloride, to give the desired product as a white solid (62% yield). FABHRMS m/z 263.1141 (M+H, C₁₂H₁₅N₄O₃ requires 263.1139).

Anal. Calcd for C₁₂H₁₄N₄O₃: C, 54.58; H, 5.29; N, 21.45. Found: C, 54.96; H, 5.38; N, 21.36.

### EXAMPLE 160

This example illustrates the production of ethyl 2-(3-cyanopropyl)-7-trityl-2,4,5,6-tetrahydropyrazolo[3,4-e]indazole-3-carboxylate.

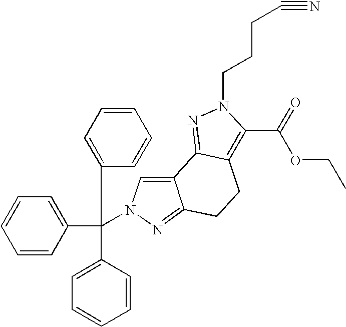

Ethyl 6-trityl-2,4,5,6-tetrahydropyrazolo[3,4-e]indazole-3-carboxylate and 4-bromobutyronitrile were reacted according to the procedure for the production of ethyl 2-(3-aminopropyl)-2,4,5,6-tetrahydropyrazolo[3,4-e]indazole-3-carboxylate dihydrochloride to give the desired product as off-white crystals (44% yield). FABHRMS m/z 542.2578 (M+H, C₃₄H₃₂N₅O₂ requires 542.2551). ¹H NMR (CDCl₃/300 MHz): 7.67 (s, 1H); 7.40-7.20 (m, 15H); 4.65 (t, 2H); 4.42 (q, 2H); 3.20-3.10 (m, 2H); 3.05-2.95 (m, 2H); 2.45-2.38 (m, 2H); 2.30-2.20 (m, 2H); 1.45 (t, 3H).

Anal. Calcd for C₃₄H₃₁N₅O₂ (0.5H₂O): C, 74.16; H, 5.86; N, 12.72. Found: C, 74.14; H, 5.98; N, 12.13.

### EXAMPLE 161

This example illustrates the production of 2-(4-aminobutyl)-2,4,5,6-tetrahydropyrazolo[3,4-e]indazole-3-carboxylic acid dihydrochloride:

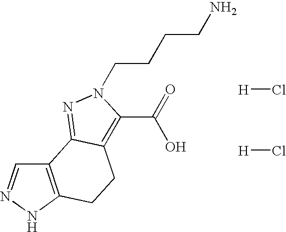

Ethyl 2-(3-cyanopropyl)-6-trityl-2,4,5,6-tetrahydropyrazolo[3,4-e]indazole-3-carboxylate (2.0 g, 0.0037 mol), di-t-butyl-dicarbonate (3.3 g, 0.015 mol), platinum oxide (721 mg) and THF (50 mL) were shaken at 55 psi H₂ on a Parr hydrogenator overnight. Contents were filtered through clay and the filtrate was concentrated in vacuo leaving a colorless oil. The oil, 2.5 N sodium hydroxide (5 mL), water (50 mL) and methanol (50 mL) were refluxed for 4 hours. Contents were allowed to cool and extracted with EtOAc. The EtOAc extract was concentrated in vacuo and the residue was triturated with 4N HCl in dioxane (25 mL) overnight. Contents were filtered to give the desired product as a white solid, 1.48 g (93% yield). FABHRMS m/z 276.1472 (M+H, C₁₃H₁₈N₅O₂ requires 276.1455). ¹H NMR (DMSO-d₆+5%TFA/300 MHz): 8.10 (s, 1H); 7.85 (br s, 3H); 4.50 (t, 2H); 3.10-3.00 (m, 2H); 2.98-2.88 (m 2H); 2.85-2.78 (m, 2H); 1.90-1.80 (m, 2H); 1.60-1.50 (m, 2H).

Anal. Calcd for C₁₃H₁₇N₅O₂ (3 HCl, 2.5H₂O): C, 36.34; H, 5.86; N, 16.30. Found: C, 36.33; H, 5.75; N, 15.72.

### EXAMPLE 162

This example illustrates the production of 2-(3-cyanopropyl)-2,4,5,6-tetrahydro-pyrazolo[3,4-e]indazole-3-carboxylic acid hydrochloride:

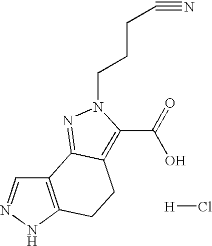

Ethyl 2-(3-cyanopropyl)-6-trityl-2,4,5,6-tetrahydropyrazolo[3,4-e]indazole-3-carboxylate (5.8 g, 0.011 mol) and 4N HCl in dioxane (40 mL) were stirred overnight. Contents were concentrated in vacuo and the residue was triturated with EtOAc and filtered to give a white solid, 3.4 g. The white solid was reacted according to the procedure for the production of 7-hydroxy-4,5-dihydro-2H-benzo[g]indazole-3-carboxylic acid to give the desired as a white solid (55% yield). FABHRMS m/z 272.1122 (M+H, C₁₃H₁₄N₅O₂ requires 272.1142).

Anal. Calcd for C₁₃H₁₃N₅O₂ (HCl, 0.2H₂O): C, 50.15; H, 4.66; N, 22.49. Found: C, 50.34; H, 4.43; N, 21.95.

### EXAMPLE 163

This example illustrates the production of ethyl 7-[(benzyloxy)methyl]-2,4,5,7-tetrahydropyrazolo[3,4-e]indazole-3-carboxylate:

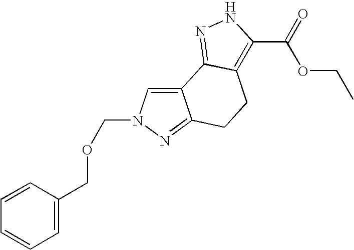

To 1,5,6,7-tetrahydro-4H-indazol-4-one (8.9 g, 0.065 mol) and benzyl chloromethyl ether (9.7 mL, 0.07 mol) in CH₂Cl₂ (500 mL) was added pyridine (10 mL) and DMAP (345 mg). Contents were stirred overnight before filtering through a pad of silica gel, eluting with 50% EtOAc/hexanes to give a mixture of 1-benzyloxymethy-1,5,6,7-tetrahydro-4H-indazol-4-one and 2-benzyloxymethy-1,5,6,7-tetrahydro-4H-indazol-4-one as a colorless oil (55% yield). To the mixture of isomers (9.2 g, 0.036 mol) and diethyl oxalate (4.9 mL, 0.036 mol) in ether (200 mL) was added dropwise lithium bis(trimethylsilyl)amide (1M in THF, 36 mL). Contents were stirred 3 hours and quenched with water. The aqueous layer was made acidic with 1N HCl to pH 2 and extracted with EtOAc, which was dried over MgSO₄ and concentrated in vacuo leaving a mixture of ethyl oxo(4-oxo-1-benzyloxymethyl-4,5,6,7-tetrahydro-1H-indazol-5-yl)acetate and ethyl oxo(4-oxo-2-benzyloxy-methyl-4,5,6,7-tetrahydro-1H-indazol-5-yl)acetate as a light amber oil, 12.8 g (100% yield). To the mixture of isomers (12.8 g, 0.036 mol) in glacial acetic acid (100 mL) was added dropwise hydrazine hydrate (1.7 mL, 0.036 mol) in glacial acetic acid (20 mL). Contents were stirred overnight and concentrated in vacuo. The residue was mixed with aqueous methanol, precipitating a solid which was filtered to give a solid (2.5 g). The solid was recrystallized from EtOAc to give the desired product as an off-white solid, 595 mg (5% yield). The aqueous methanolic filtrate was purified in ethyl 6-[(benzyloxy)methyl]-2,4,5,6-tetrahydropyrazolo[3,4-e]indazole-3-carboxylate below. FABHRMS m/z 353.1619 (M+H, C₁₉H₂₁N₄O₃ requires 353.1614). ¹H NMR (CDCl₃/300 MHz): 9.20 (brs, 1H); 7.90 (s, 1H); 7.40-7.20 (m, 5H); 5.50 (s, 2H); 4.60 (s, 2H); 4.40 (q, 2H); 3.20-2.95 (m, 4H); 1.40 (t, 3H).

Anal. Calcd for C₁₉H₂₀N₄O₃: C, 64.75; H, 5.64; N, 15.90. Found: C, 64.76; H, 5.72; N, 15.90.

### EXAMPLE 164

This example illustrates the production of ethyl 6-[(benzyloxy)methyl]-2,4,5,6-tetrahydropyrazolo[3,4-e]indazole-3-carboxylate:

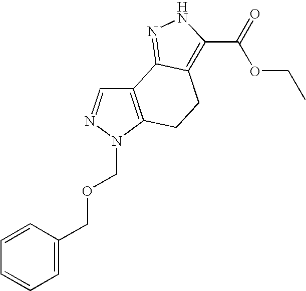

The aqueous methanolic filtrate from ethyl 7-[(benzyloxy)methyl]-2,4,5,7-tetrahydropyrazolo[3,4-e]indazole-3-carboxylate was purified by silica gel chromatography eluting with 35% EtOAc/hexanes to give after recrystallization from EtOAc/hexanes, the desired product as a white solid, 700 mg (6% yield). FABHRMS m/z 353.1614 (M+H, C₁₉H₂₁N₄O₃ requires 353.1623). ¹H NMR (CDCl₃/300 MHz): 9.40 (br s, 1H); 7.92 (s, 1H); 7.40-7.20 (m, 5H); 5.60 (s, 2H); 4.60 (s, 2H); 4.40 (q, 2H); 3.25-3.00 (m, 4H); 1.45 (t, 3H).

Anal. Calcd for C₁₉H₂₁N₄O₃: C, 64.76; H, 5.72; N, 15.90. Found: C, 64.49; H, 5.50; N, 15.84.

EXAMPLE 165.

This example illustrates the production of 2,4,5,6-tetrahydropyrazolo[3,4-e]indazole-3-carboxylic acid hydrochloride

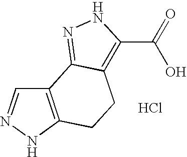

A mixture of ethyl 7-[(benzyloxy)methyl]-2,4,5,7-tetrahydropyrazolo[3,4-e]indazole-3-carboxylate and ethyl 6-[(benzyloxy)methyl]-2,4,5,6-tetrahydropyrazolo[3,4-e]indazole-3-carboxylate (1.1 gm, 0.0032 mol) was heated in 3N HCl overnight. Contents were allowed to cool and evaporated by half under a stream of N₂, then filtered to give the desired product as a white solid, 385 mg (50% yield). FABHRMS m/z 205.0704 (M+H, C₉H₉N₄O₂ requires 205.0726).

Anal. Calcd for C₉H₈N₄O₂ (1 HCl, 0.4H₂O): C, 43.61; H, 3.99; N, 22.60. Found: C, 43.81; H, 4.40; N, 22.75.

### EXAMPLE 166

This example illustrates the production of 2,6-dihydropyrazolo[3,4-e]indazole-3-carboxylic acid hydrochloride:

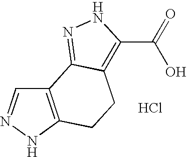

A mixture of ethyl 7-[(benzyloxy)methyl]-2,4,5,7-tetrahydropyrazolo[3,4-e]indazole-3-carboxylate and ethyl 6-[(benzyloxy)methyl]-2,4,5,6-tetrahydropyrazolo[3,4-e]indazole-3-carboxylate (5.3 g, 0.015 mol) and DDQ (3.4 g, 0.015 mol) in dioxane was refluxed 5 hours. Contents were allowed to cool and the by-product was filtered. The filtrate was concentrated in vacuo leaving an amber foam (4.6 g). The amber foam, 3N HCl (50 mL) and methanol (25 mL) were refluxed overnight. Contents were allowed to cool and concentrated in vacuo. The residue was triturated with methanol and filtered to give the desired product as a light amber solid, 1.2 g (41% yield). FABHRMS m/z 203.0578 (M+H, C₉H₇N₄O₂ requires 203.0564). ¹H NMR (DMSO-d₆/300 MHz): 8.30 (s, 1H); 8.00 (d, 1H); 7.43 (d, 1H);

Anal. Calcd for C₉H₆N₄O₂ (0.9 HCl): C, 46.00; H, 2.96; N, 23.84. Found: C, 46.21; H, 3.34; N, 23.94.

### EXAMPLE 167

This example illustrates the production of 1,6-dihydropyrazolo[3,4-e]indazole-3-carboxamide:

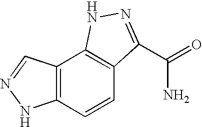

A mixture of ethyl 7-[(benzyloxy)methyl]-2,4,5,7-tetrahydropyrazolo[3,4-e]indazole-3-carboxylate and ethyl 6-[(benzyloxy)methyl]-2,4,5,6-tetrahydropyrazolo[3,4-e]indazole-3-carboxylate (1.7 g, 0.005 mol) and DDQ (1.1 g, 0.005 mol) in dioxane was refluxed 5 hours. Contents were allowed to cool and the by-product was filtered. The filtrate was concentrated in vacuo leaving an amber foam. The amber foam, TFA (20 mL) and CH₂Cl₂ (20 mL) were refluxed overnight. Contents were allowed to cool and concentrated in vacuo leaving an amber solid. The residue was triturated with methanol and filtered to give ethyl 2,6-dihydropyrazolo[3,4-e]indazole-3-carboxylate as a light amber solid, 1.3 g (76% yield). The solid (990 mg, 0.003 mol), conc ammonium hydroxide (30 mL) and methanol (15 mL) were stirred in a stoppered flask for 6 days. Contents were poured into a beaker to let the volatiles evaporate and filtered to give a tan solid. The solid was triturated with methanol and filtered to give the desired product as a tan solid, 577 mg (96% yield). (FABHRMS m/z 202.0725 (M+H, C₉H₈N₅O requires 202.0723).

¹H NMR (DMSO-d₆+TFA/300 MHz): 8.26 (s, 1H); 8.12 (d, 1H); 7.40 (d, 1H).

Anal. Calcd for C₉H₇N₅O (0.7H₂O): C, 50.56; H, 3.96; N, 32.76. Found: C, 50.87; H, 4.11; N, 29.74.

### EXAMPLE 168

This example illustrates the production of 2-allyl-2,4,5,6-tetrahydropyrazolo[3,4-e]indazole-3-carboxylic acid hydrochloride:

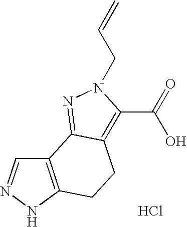

To ethyl 7-[(benzyloxy)methyl]-2,4,5,7-tetrahydropyrazolo[3,4-e]indazole-3-carboxylate (1.8 g, 0.005 mol) in DMF (15 mL) was added a 60% dispersion of sodium hydride in mineral oil (204 mg). Contents were stirred 2 hours and allyl bromide (0.512 mL, 0.006 mol) was added. Contents were stirred overnight, quenched with water, extracted with EtOAc, dried over MgSO₄ and concentrated in vacuo leaving an oil. The oil was purified by silica gel chromatography eluting with 50% EtOAc/hexanes. Fr 2-3 gave ethyl 2-allyl-2,4,5,6-tetrahydropyrazolo-[3,4-e]indazole-3-carboxylate as a yellow oil (344 mg). Fr 4-6 gave ethyl 1-allyl-2,4,5,6-tetrahydropyrazolo[3,4-e]indazole-3-carboxylate as a yellow oil (671 mg). Fr 2-3 and 6N HCl were refluxed overnight, allowed to cool and concentrated in vacuo. The residue was triturated with methanol and filtered to give the desired product as a white solid (80% yield). FABHRMS m/z 245.1050 (M+H, C₁₂H₁₃N₄O₂ requires 245.1033). ¹H NMR (DMSO-d₆+TFA/300 MHz): 8.15 (s, 1H); 6.10-5.90 (m, 1H); 5.20-4.93 (m, 4H); 3.10-2.90 (m, 4H).

Anal. Calcd for C₁₂H₁₂N₄O₂ (HCl, H₂O): C, 48.25; H, 5.06; N, 18.75. Found: C, 48.28; H, 4.90; N, 18.67.

### EXAMPLE 169

This example illustrates the production of 2-allyl-2,4,5,6-tetrahydropyrazolo[3,4-e]indazole-3-carboxamide:

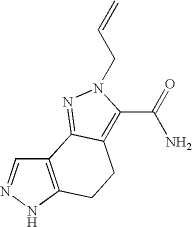

Ethyl 2-allyl-2,4,5,6-tetrahydropyrazolo[3,4-e]indazole-3-carboxylate, prepared in 2-allyl-2,4,5,6-tetrahydropyrazolo[3,4-e]indazole-3-carboxylic acid hydrochloride was reacted according to the procedure for the production of 8-hydroxy-4,5-dihydro-2H-benzo[g]indazole-3-carboxamide, to give the desired product as a white solid (60% yield). FABHRMS m/z 244.1159 (M+H, C₁₂H₁₄N₅O requires 244.1193).

Anal. Calcd for C₁₂H₁₃N₅O (0.25H₂O): C, 58.17; H, 5.49; N, 28.27. Found: C, 58.54; H, 5.86; N, 27.81.

### EXAMPLE 170

This example illustrates the production of ethyl 2-allyl-7-trityl-2,4,5,6-tetrahydropyrazolo[3,4-e]indazole-3-carboxylate:

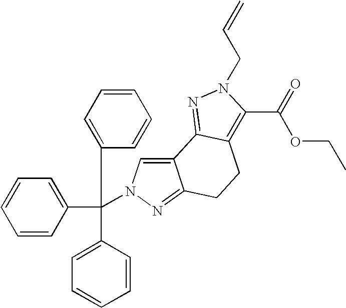

Ethyl 6-trityl-2,4,5,6-tetrahydropyrazolo[3,4-e]indazole-3-carboxylate was reacted according to the procedure for the production of ethyl 2-(3-aminopropyl)-2,4,5,6-tetrahydropyrazolo[3,4-e]indazole-3-carboxylate dihydrochloride to give the desired product as a white solid (33% yield).

Anal. Calcd for C₃₃H₃₀N₄O₂: C, 77.02; H, 5.88; N, 10.89. Found: C, 76.98; H, 5.88; N, 10.72.

### EXAMPLE 171

This example illustrates the production of 1-allyl-1,4,5,6-tetrahydro-pyrazolo[3,4-e]indazole-3-carboxylic acid hydrochloride:

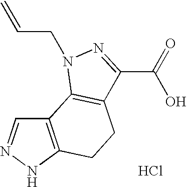

Ethyl 1-allyl-2,4,5,6-tetrahydropyrazolo[3,4-e]indazole-3-carboxylate, obtained from Fr 4-6 of 2-allyl-2,4,5,6-tetrahydropyrazolo[3,4-e]indazole-3-carboxylic acid hydrochloride and 6N HCl were refluxed overnight, allowed to cool and concentrated in vacuo. The residue was triturated with methanol and filtered to give the desired product as a white solid (87% yield). FABHRMS m/z 245.1052 (M+H, C₁₂H₁₃N₄O₂ requires 245.1033).

Anal. Calcd for C₁₂H₁₂N₄O₂ (HCl): C, 51.35; H, 4.67; N, 19.96. Found: C, 51.23; H, 4.77; N, 19.52.

### EXAMPLE 172

This example illustrates the production of 1-allyl-1,4,5,6-tetrahydro-pyrazolo[3,4-e]indazole-3-carboxamide:

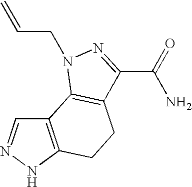

Ethyl 1-allyl-2,4,5,6-tetrahydropyrazolo[3,4-e]indazole-3-carboxylate, prepared in 1-allyl-1,4,5,6-tetrahydropyrazolo[3,4-e]indazole-3-carboxylic acid hydrochloride was reacted according to the procedure for the production of 8-hydroxy-4,5-dihydro-2H-benzo[g]indazole-3-carboxamide, to give the desired product as a white solid (65% yield). FABHRMS m/z 244.1179 (M+H, C₁₂H₁₄N₅O requires 244.1193). ¹HNMR (DMSO-d₆+TFA/300 MHz): 8.00 (s, 1H); 6.10-5.95 (m, 1H); 5.20-4.85 (m, 4H); 3.10-2.80 (m, 4H).

Anal. Calcd for C₁₂H₁₃N₅O (0.3H₂O): C, 57.96; H, 5.51; N, 28.16. Found: C, 57.95; H, 5.22; N, 26.32.

### EXAMPLE 173

This example illustrates the production of 4,5-dibromo-2,6-dihydropyrazolo[3,4-e]indazole-3-carboxylic acid:

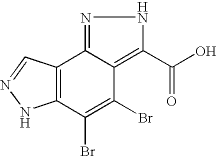

To ethyl 6-trityl-2,4,5,6-tetrahydropyrazolo[3,4-e]indazole-3-carboxylate (1.0 g, 0.002 mol) and sodium acetate (230 mg) in glacial acetic acid was added dropwise, bromine (0.380 mL, 0.0074 mol). Contents were heated at 90° C. for 3 hours, allowed to cool, and filtered to yield a yellow solid. The yellow solid was triturated with CH₂Cl₂ and filtered to give ethyl 4,5-dibromo-2,6-dihydropyrazolo[3,4-e]indazole-3-carboxylate as a white solid. Ethyl 4,5-dibromo-2,6-dihydropyrazolo[3,4-e]indazole-3-carboxylate, 2.5N sodium hydroxide (0.5 mL), water (0.5 mL) and methanol (5 mL) were stirred overnight. Contents were made acidic to pH 2 with 1N HCl and filtered to give the desired product as a white solid, 310 mg (60% yield). FABHRMS m/z 358.8586 (M+H, C₉H₅Br₂N₄O₂ requires 358.8597). ¹H NMR (DMSO-d₆+TFA/300 MHz): 8.20 (s, 1H).

Anal. Calcd for C₉H₄Br₂N₄O₂ (0.8H₂O): C, 28.87; H, 1.51; N, 14.97. Found: C, 28.96; H, 1.73; N, 14.77.

### EXAMPLE 174

This example illustrates the production of 2-[bis(benzylthio) methylenelcyclohexane-1,3-dione:

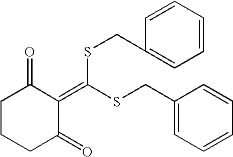

To 1,3-cyclohexanedione (11.2 g, 0.1 mol) in DMF (100 mL) was added CS₂ (10 mL, 0.17 mol), and potassium carbonate (41.4 g, 0.3 mol). Contents were stirred for 3 hours and benzyl chloride (23 mL, 0.2 mol) was added dropwise. Contents were stirred overnight. Contents were partitioned between water and EtOAc. The EtOAc layer was dried over MgSO₄ and concentrated in vacuo. The residue was filtered through a plug of silica gel, eluting with 25% EtOAc/hexanes. Crystals grew in the filtrate and were filtered to give the desired product as orange flakes, 9.4 g (26% yield). FABHRMS m/z 369.0972 (M+H, C₂₁H₂₁O₂S requires 369.0978). ¹H NMR (CDCl₃/300 MHz): 7.40-7.25 (m, 10H); 4.20 (s, 4H); 2.60 (t, 4H); 1.95 (quintet, 2H).

Anal. Calcd for C₂₁H₂₀O₂S: C, 68.44; H, 5.47. Found: C, 68.29; H, 5.37.

### EXAMPLE 175

This example illustrates the production of 3-(benzylthio)-1-[(4-methylphenyl)sulfonyl]-1,5,6,7-tetrahydro-4H-indazol-4-one:

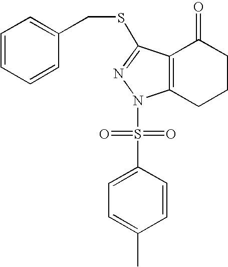

2-[bis(benzylthio)methylene]cyclohexane-1,3-dione (7.0 g, 0.019 mol) and p-toluenesulfonhydrazide (3.9 g, 0.021 mol) in methanol (100 mL) were stirred overnight. Contents were filtered to give the desired product as a white solid, 6.2 g (79% yield). FABHRMS m/z 413.0970 (M+H, C₂₁H₂₁N₂O₃S₂ requires 413.0988). ¹H NMR (CDCl₃/300 MHz): 7.88 (d, 2H); 7.43-7.27 (m, 7H); 4.35 (s, 3H); 3.24 (t, 2H); 2.45 (s, 3H); 2.43 (t, 2H); 2.20 (quintet, 2H).

Anal. Calcd for C₂₁H₂₀N₂O₃S₂: C, 60.98; H, 4.75; N, 6.79. Found: C, 61.14; H, 4.89; N, 6.79.

### EXAMPLE 176

This example illustrates the production of ethyl {3-(benzylthio)-1-[(4-methylphenyl)sulfonyl]-4-oxo-4,5,6,7-tetrahydro-1H-indazol-5-yl}(oxo)acetate:

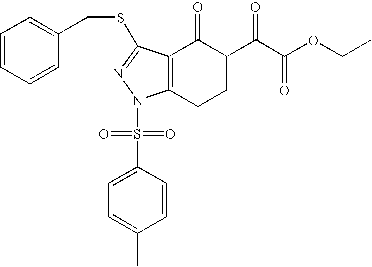

3-(benzylthio)-1-[(4-methylphenyl)sulfonyl]-1,5,6,7-tetrahydro-4H-indazol-4-one was reacted according to the procedure for the production of ethyl (7-hydroxy-1-oxo-1,2,3,4-tetrahydronaphthalen-2-yl)(oxo)acetate to give the desired product as a yellow solid (87% yield). FABHRMS m/z 513.1179 (M+H, C₂₅H₂₅N₂O₆S₂ requires 513.1149). ¹H NMR (CDCl₃/300 MHz): 7.90 (d, 2H); 7.45-7.25 (m, 7H); 4.42-4.35 (m, 4H); 3.30-3.15 (m, 4H); 2.48 (s, 3H); 1.40 (t, 3H).

Anal. Calcd for C₂₅H₂₄N₂O₆S₂: C, 58.58; H, 4.72; N, 5.46. Found: C, 58.63; H, 4.51; N, 5.83.

### EXAMPLE 177

This example illustrates the production of ethyl 8-(benzylthio)-2,4,5,6-tetrahydropyrazolo[3,4-e]indazole-3-carboxylate:

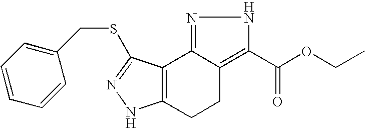

Ethyl {3-(benzylthio)-1-[(4-methylphenyl)sulfonyl]-4-oxo-4,5,6,7-tetrahydro-1H-indazol-5-yl}(oxo)acetate and hydrazine monohydrochloride were reacted according to the procedure for the production of ethyl 8-hydroxy-4,5-dihydro-2H-benzo[g]indazole-3-carboxylate, refluxing in methanol, to give the desired product as yellow solid (38% yield). FABHRMS m/z 355.1220 (M+H, C₁₈H₁₉N₄O₂S requires 355.1223). ¹H NMR (DMSO-d₆+5%TFA/300 MHz): 7.30-7.20 (m, 5H); 4.38 (s, 2H); 4.35 (q, 2H); 3.05 (t, 2H); 2.85 (t, 2H); 1.35 (t, 3H).

Anal. Calcd for C₁₈H₁₈N₄O₂S: C, 61.00; H, 5.12; N, 15.81. Found: C, 60.53; H, 4.91; N, 15.38.

### EXAMPLE 178

This example illustrates the production of 8-(benzylthio)-2,4,5,6-tetrahydropyrazolo [3,4-e]indazole-3-carboxamide:

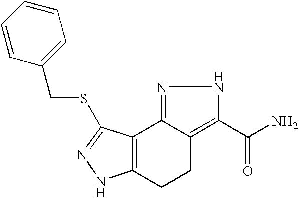

Ethyl 8-(benzylthio)-2,4,5,6-tetrahydropyrazolo[3,4-e]indazole-3-carboxylate was reacted according to the procedure for the production of 8-hydroxy-4,5-dihydro-2H-benzo[g]indazole-3-carboxamide to give the desired as an off-white solid (60% yield). FABHRMS m/z 326.1065 (M+H, C₁₆H₁₆N₅OS requires 326.1070). ¹H NMR (DMSO-d₆+5%TFA/300 MHz): 7.40 (br, 1H); 7.30-7.15 (m, 5H); 4.35 (s, 2H); 3.05 (t, 2H); 2.80 (t, 2H).

Anal. Calcd for C₁₆H₁₅N₅OS (0.6H₂O): C, 57.16; H, 4.86; N, 20.83. Found: C, 57.11; H, 4.73; N, 20.68.

### EXAMPLE 179

This example illustrates the production of 8-(benzylthio)-2,4,5,6-tetrahydropyrazolo[3,4-e]indazole-3-carboxylic acid:

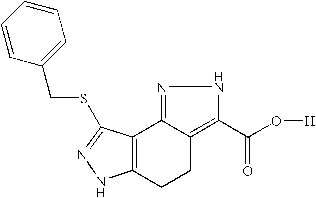

Ethyl 8-(benzylthio)-2,4,5,6-tetrahydropyrazolo[3,4-e]indazole-3-carboxylate was reacted according to the procedure for the production of 7-hydroxy-4,5-dihydro-2H-benzo[g]indazole-3-carboxylic acid to give the desired as an off-white solid (53% yield). FABHRMS m/z 327.0930 (M+H, C₁₆H₁₅N₄O₂S requires 327.0910). ¹H NMR (CDCl₃/300 MHz)

Anal. Calcd for C₁₆H₁₄N₄O₂S: C, 58.88; H, 4.32; N, 17.17. Found: C, 58.34; H, 4.62; N, 15.97.

### EXAMPLE 180

This example illustrates the production of 2-(3-t-butoxycarbonylaminopropyl)-8-(benzylthio)-2,4,5,6-tetrahydropyrazolo[3,4-e]indazole-3-carboxylic acid:

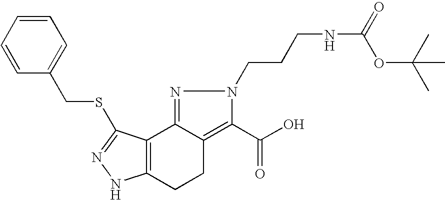

Ethyl 8-(benzylthio)-6-[(4-methylphenyl)sulfonyl]-2,4,5,6-tetrahydropyrazolo[3,4-e]indazole-3-carboxylate was prepared according to the procedure for the production of ethyl 8-hydroxy-4,5-dihydro-2H-benzo[g]indazole-3-carboxylate, using ethyl {3-(benzylthio)-1-[(4-methylphenyl)sulfonyl]-4-oxo-4,5,6,7-tetrahydro-1H-indazol-5-yl}(oxo)acetate and reacted according to the procedure for the production of ethyl 2-(3-aminopropyl)-2,4,5,6-tetrahydropyrazolo[3,4-e]indazole-3-carboxylate dihydrochloride, to give a red oil. The red oil was reacted according to the procedure for the production of 7-hydroxy-4,5-dihydro-2H-benzo[g]indazole-3-carboxylic acid to give the desired product after trituration with EtOAc, as a white solid (50% yield). FABHRMS m/z 484.1999 (M+H, C₂₄H₃₀N₅O₄S requires 484.2013). ¹H NMR (DMSO-d₆+TFA/300 MHz): 7.30-7.15 (m, 5H); 4.60-4.45 (m, 2H); 4.38 (s, 2H); 3.10-2.93 (m, 4H); 2.90-2.80 (m, 2H); 2.00-1.85 (m, 2H); 1.40 (s, 9H).

Anal. Calcd for C₂₄H₂₉N₅O₄S: C, 59.61; H, 6.04; N, 14.48. Found: C, 59.34; H, 6.09; N, 14.02.

### EXAMPLE 181

This example illustrates the production of 2-(3-aminopropyl)-8-(benzylthio)-2,4,5,6-tetrahydropyrazolo[3,4-e]indazole-3-carboxylic acid dihydrochloride:

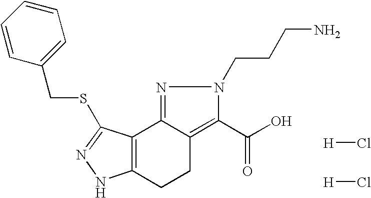

Ethyl 2-{3-[(tert-butoxycarbonyl)amino]propyl}-8-(benzylthio)-2,4,5,6-tetrahydropyrazolo[3,4-e]indazole-3-carboxylate (171 mg, 0.35 mmol) and 4N HCl in dioxane (10 mL) were stirred overnight, diluted with EtOAc and filtered to give a white solid, 137 mg (86% yield). FABHRMS m/z 384.1457 (M+H, C₁₉H₂₂N₅O₂S requires 384.1489). ¹H NMR (DMSO-d₆+TFA/300 MHz): 7.80 (br, 3H); 7.30-7.20 (m, 5H); 4.60 (t, 2H); 4.40 (s, 2H); 3.10-3.00 (m, 2H); 2.90-2.80 (m, 4H); 2.20-2.00 (m, 2H).

Anal. Calcd for C₁₉H₂₁N₅O₂S (2HCl): C, 50.00; H, 5.08; N, 15.35. Found: C, 49.66; H, 5.02; N, 15.00.

### EXAMPLE 182

This example illustrates the production of ethyl 2-(3-aminopropyl)-8-(benzylthio)-2,4,5,6-tetrahydropyrazolo[3,4-e]indazole-3-carboxylate dihydrochloride:

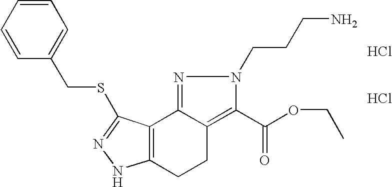

Ethyl 8-(benzylthio)-6-[(4-methylphenyl)sulfonyl-2,4,5,6-tetrahydropyrazolo[3,4-e]indazole-3-carboxylate was prepared according to the procedure for the production of ethyl 8-hydroxy-4,5-dihydro-2H-benzo[g]indazole-3-carboxylate using ethyl 2-{3-[(tert-butoxycarbonyl)amino]propyl}-8-(benzylthio)-2,4,5,6-tetrahydropyrazolo[3,4-e]indazole-3-carboxylate and reacted according to the procedure for the production of ethyl 2-(3-aminopropyl)-2,4,5,6-tetrahydropyrazolo[3,4-e]indazole-3-carboxylate dihydrochloride, to give a red oil. The red oil was mixed with 4N HCl in dioxane and stirred overnight, then filtered to give the desired product after trituration with EtOAc, as a white solid (80% yield). FABHRMS m/z 412.1796 (M+H, C₂₁H₂₆N₅O₂S requires 412.1802). ¹H NMR (DMSO-d₆+TFA/300 MHz): 7.80 (br, 3H); 7.35-7.10 (m, 5H); 4.60-4.50 (m, 2H); 4.35-4.25 (m, 4H); 3.10-3.00 (m, 2H); 2.90-2.80 (m, 4H); 2.20-2.00 (m, 2H); 1.40-1.30 (m, 3H).

Anal. Calcd for C₂₁H₂₅N₅O₄S (2HCl): C, 52.06; H, 5.62; N, 14.46. Found: C, 52.22; H, 6.18; N, 14.62.

### EXAMPLE 183

This example illustrates the production of 1-(benzylthio)-4,5,7,8,9,10-hexahydro[1,4]diazepino[1,2-b]pyrazolo[3,4-g]indazol-6(3H)-one:

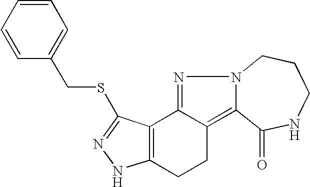

Ethyl 2-(3-aminopropyl)-8-(benzylthio)-2,4,5,6-tetrahydropyrazolo[3,4-e]indazole-3-carboxylate dihydrochloride (1.0 g, 0.0024 mol), conc ammonium hydroxide (30 mL) and methanol (15 mL) were stirred in a stoppered flask for 72 hours. Contents were poured into a beaker to allow volatiles to evaporate and then filtered to give the desired product as a pale green solid, 310 mg (35% yield). FABHRMS m/z 366.1386 (M+H, C₁₉H₂₀N₅O₂S requires 366.1383). ¹H NMR (DMSO-d₆+TFA/300 MHz): 8.20 (br s, 1H); 7.35-7.20 (m, 5H); 4.45-4.35 (m, 4H); 3.25-3.15 (m, 2H); 3.00-2.80 (m, 4H); 2.20-2.10 (m, 2H).

Anal. Calcd for C₁₉H₁₉N₅O₄S(H₂O): C, 59.51; H, 5.52; N, 18.26. Found: C, 59.77; H, 5.19; N, 18.26.

### EXAMPLE 184

This example illustrates the production of 1-[(4-methylphenyl)sulfonyl]-3-(methylthio)-1,5,6,7-tetrahydro-4H-indazol-4-one:

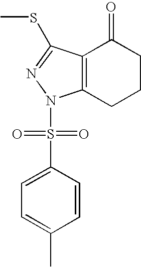

2-[bis(methylthio)methylene]cyclohexane-1,3-dione was prepared according to the procedure of 2-[bis(benzylthio)methylene]cyclohexane-1,3-dione, using methyl iodide (28% yield) and reacted according to the procedure of 3-(benzylthio)-1-[(4-methylphenyl)sulfonyl]-1,5,6,7-tetrahydro-4H-indazol-4-one, to give the desired product as a white solid (39% yield). FABHRMS m/z 337.0658 (M+H, C₁₅H₁₇N₂O₃S₂ requires 337.0675). ¹H NMR (CDCl₃/300 MHz): 7.95 (d, 2H); 7.39 (d, 2H); 3.24 (t, 2H); 2.52 (s, 3H); 2.58-2.44 (m, 2H); 2.46 (s, 3H); 2.23-2.10 (m, 2H).

Anal. Calcd for C₁₅H₁₆N₂O₃S₂: C, 53.55; H, 4.79; N, 8.33. Found: C, 53.38; H, 4.80; N, 8.48.

### EXAMPLE 185

This example illustrates the production of ethyl [1-[(4-methylphenyl)sulfonyl]-3-(methylthio)-4-oxo-4,5,6,7-tetrahydro-1H-indazol-5-yl](oxo)acetate:

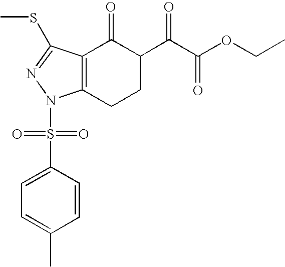

1-[(4-methylphenyl)sulfonyl]-3-(methylthio)-1,5,6,7-tetrahydro-4H-indazol-4-one was reacted according to the procedure for the production of ethyl (7-hydroxy-1-oxo-1,2,3,4-tetra hydronaphthalen-2-yl)(oxo)acetate to give the desired as a yellow solid (79% yield). ¹H NMR (CDCl₃/300 MHz): 7.95 (d, 1H); 7.40 (d, 1H); 4.40 (q, 2H); 3.35-3.20 (m, 4H); 2.55 (s, 3H); 2.50 (s, 3H); 1.44 (t, 3H).

Anal. Calcd for C₁₉H₂₀N₂O₆S₂ (0.5H₂O): C, 51.22; H, 4.75; N, 6.29. Found: C, 51.10; H, 5.08; N, 6.52.

### EXAMPLE 186

This example illustrates the production of ethyl 2-{3-[(tert-butoxycarbonyl) amino]propyl}-6-[(4-methylphenyl)sulfonyl]-8-(methylthio)-2,4,5,6-tetrahydropyrazolo[3,4-e]indazole-3-carboxylate:

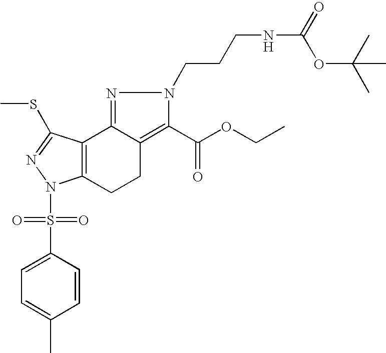

Ethyl 8-(methylthio)-6-[(4-methylphenyl)sulfonyl]-2,4,5,6-tetrahydropyrazolo[3,4-e]indazole-3-carboxylate was prepared according to the procedure for the production of ethyl 8-hydroxy-4,5-dihydro-2H-benzo[g]indazole-3-carboxylate, using ethyl [1-[(4-methylphenyl)sulfonyl]-3-(methylthio)-4-oxo-4,5,6,7-tetrahydro-1H-indazol-5-yl](oxo)acetate, and hydrazine monohydrochloride and reacted according to the procedure for the production of ethyl 2-(3-aminopropyl)-2,4,5,6-tetrahydropyrazolo[3,4-e]indazole-3-carboxylate dihydrochloride to give the desired product as a white solid (31% yield). FABHRMS m/z 590.2120 (M+H, C₂₇H₃₅N₅O₆S₂ requires 590.2102). ¹H NMR (DMSO-d₆+5%TFA/300 MHz): 7.85 (d, 2H); 7.42 (d, 2H); 6.79 (br, 1H); 4.45-4.40 (m, 2H); 4.30 (q, 2H); 3.35-3.30 (m, 2H); 3.10-3.05 (m, 2H); 2.98-2.85 (m, 2H); 2.40 (s, 3H); 1.85-1.80 (m, 2H); 1.35 (s, 9H); 1.33 (t, 3H).

Anal. Calcd for C₂₇H₃₅N₅O₆S₂: C, 54.66; H, 5.88; N, 11.69. Found: C, 54.99; H, 5.98; N, 11.88.

### EXAMPLE 187

This example illustrates the production of ethyl 2-(3-aminopropyl)-8-(methylthio)-2,4,5,6-tetrahydropyrazolo[3,4-e]indazole-3-carboxylate dihydrochloride:

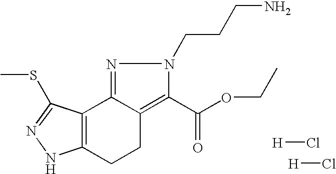

Ethyl 2-{3-[(tert-butoxycarbonyl)amino]propyl}-6-[(4-methylphenyl)-sulfonyl]-8-(methylthio)-2,4,5,6-tetrahydropyrazolo[3,4-e]indazole-3-carboxylate (900 mg, 0.0015 mol) and 4 N HCl in dioxane (10 mL) were stirred overnight, diluted with ether (10 mL) and filtered to give the desired as an off-white solid, 628 mg (95% yield). FABHRMS m/z 336.1476 (M+H, C₁₅H₂₂N₅O₂S requires 336.1489). ¹H NMR (DMSO-d₆+5%TFA/300 MHz): 4.55 (t, 2H); 4.35 (q, 2H); 3.05-3.00 (m, 2H); 2.90-2.80 (m, 4H); 2.55 (s, 3H); 2.15-2.00 (m, 2H); 1.37 (t, 3H).

Anal. Calcd for C₁₅H₂₁N₅O₂S (2HCl): C, 44.12; H, 5.68; N, 17.15. Found: C, 42.97; H, 5.60; N, 16.70.

### EXAMPLE 188

This example illustrates the production of 1-(methylthio)-4,5,7,8,9,10-hexahydro [1,4]diazepino[1,2-b]pyrazolo[3,4-g]indazol-6(3H)-one:

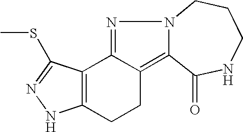

Ethyl 2-(3-aminopropyl)-8-(methylthio)-2,4,5,6-tetrahydropyrazolo[3,4-e]indazole-3-carboxylate dihydrochloride (400 mg, 1.0 mmol), conc. ammonium hydroxide (30 mL) and methanol (15 mL) were stirred in a stoppered flask overnight. Contents were poured into a beaker to let the volatiles evaporate and filtered to give the desired product as a light amber solid, 183 mg (63% yield). FABHRMS m/z 290.1074 (M+H, C₁₃H₁₆N₅OS requires 290.1070). ¹H NMR (DMSO-d₆+5%TFA/30O MHz): 8.19 (br, 1H); 4.38 (t, 2H); 3.20 (q, 2H); 2.95-2.80 (m, 4H); 2.55 (s, 3H); 2.10 (quintet, 2H).

Anal. Calcd for C₁₃H₁₅N₅OS: C, 53.96; H, 5.23; N, 24.20. Found: C, 53.72; H, 5.15; N, 24.19.

### EXAMPLE 189

This example illustrates the production of ethyl 8-(methylthio)-2,4,5,6-tetrahydro-pyrazolo[3,4-e]indazole-3-carboxylate:

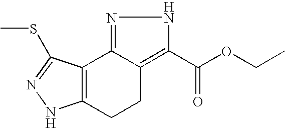

Ethyl [1-[(4-methylphenyl)sulfonyl]-3-(methylthio)-4-oxo-4,5,6,7-tetrahydro-1H-indazol-5-yl](oxo)acetate and hydrazine monohydrochloride were reacted according to the procedure for the production of ethyl 8-hydroxy-4,5-dihydro-2H-benzo[g]indazole-3-carboxylate, in refluxing methanol, to give the desired product (90% yield). FABHRMS m/z 279.0881 (M+H, C₁₂H₁₅N₄O₂S requires 279.0910). ¹H NMR (DMSO-d₆+5%TFA/300 MHz): 4.36 (q, 2H); 3.05 (t, 2H); 2.90 (t, 2H); 2.55 (s, 3H); 1.38 (t, 3H).

Anal. Calcd for C₁₂H₁₄N₄O₂S(HCl): C, 45.79; H, 4.80; N, 17.80. Found: C, 45.70; H, 4.65; N, 17.72.

### EXAMPLE 190

This example illustrates the production of 8-(methylthio)-2,4,5,6-tetrahydro-pyrazolo[3,4-e]indazole-3-carboxamide:

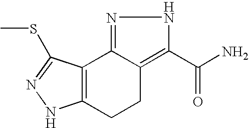

Ethyl 8-(methylthio)-2,4,5,6-tetrahydropyrazolo[3,4-e]indazole-3-carboxylate was reacted according to the procedure for the production of 8-hydroxy-4,5-dihydro-2H-benzo[g]indazole-3-carboxamide to give the desired product as a white solid (85% yield). FABHRMS m/z 250.0774 (M+H, C₁₀H₁₂N₅OS requires 250.0757). ¹H NMR (DMSO-d₆+5%TFA/300 MHz): 7.40 (br, 2H); 3.05 (t, 2H); 2.85 (t, 2H); 2.55 (s, 3H).

Anal. Calcd for C₁₀H₁₁N₅OS: C, 48.18; H, 4.45; N, 28.09. Found: C, 47.87; H, 4.36; N, 27.78.

### EXAMPLE 191

This example illustrates the production of 8-(methylthio)-2,4,5,6-tetrahydro-pyrazolo[3,4-e]indazole-3-carboxylic acid:

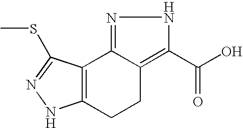

Ethyl 8-(methylthio)-2,4,5,6-tetrahydropyrazolo[3,4-e]indazole-3-carboxylate was reacted according to the procedure for the production of 7-hydroxy-4,5-dihydro-2H-benzo[g]indazole-3-carboxylic acid to give the desired as a light amber solid (82% yield). FABHRMS m/z 251.0571 (M+H, C₁₀H₁₁N₄O₂S requires 251.0597).

Anal. Calcd for C₁₀H₁₀N₄O₂S (1.6H₂O): C, 43.03; H, 4.77; N, 20.07. Found: C, 42.73; H, 4.46; N, 20.40.

### EXAMPLE 192

This example illustrates the production of ethyl 8-(methylsulfinyl)-2,4,5,6-tetrahydropyrazolo[3,4-e]indazole-3-carboxylate:

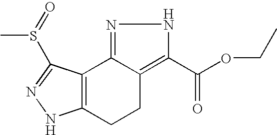

To a slurry of ethyl 8-(methylthio)-2,4,5,6-tetrahydropyrazolo[3,4-e]indazole-3-carboxylate (838 mg, 0.003 mol) in THF (20 mL) was added dropwise m-chloroperbenzoic acid (68%, 764 mg, 0.003 mol) in THF (5 mL). Contents were stirred overnight and filtered to give the desired product as a white solid, 455 mg (52% yield). FABHRMS m/z 295.0842 (M+H, C₁₂H₁₅N₄O₃S requires 295.0859).

Anal. Calcd for C₁₂H₁₄N₄O₃S: C, 48.97; H, 4.79; N, 19.04. Found: C, 45.11; H, 4.68; N, 16.96.

### EXAMPLE 193

This example illustrates the production of 8-(methylsulfinyl)-2,4,5,6 tetrahydro-pyrazolo[3,4-e]indazole-3-carboxylic acid:

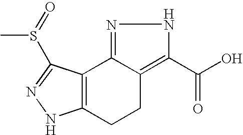

Ethyl 8-(methylsulfinyl)-2,4,5,6-tetrahydropyrazolo[3,4-e]indazole-3-carboxylate was reacted according to the procedure for the production of 7-hydroxy-4,5-dihydro-2H-benzo[g]indazole-3-carboxylic acid to give the desired product as a white solid (71% yield). FABHRMS m/z 267.0541 (M+H, C₁₀H₁₁N₄O₃S requires 267.0546).

Anal. Calcd for C₁₀H₁₀N₄O₃S(H₂O): C, 42.25; H, 4.25; N, 19.71. Found: C, 42.36; H, 3.99; N, 19.71.

### EXAMPLE 194

This example illustrates the production of ethyl 8-(methylsulfonyl)-2,4,5,6-tetrahydropyrazolo[3,4-e]indazole-3-carboxylate:

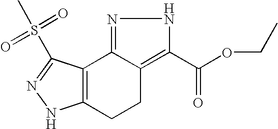

Ethyl 8-(methylsulfonyl)-2,4,5,6-tetrahydropyrazolo[3,4-e]indazole-3-carboxylate was prepared according to the procedure of ethyl 8-(methylsulfinyl)-2,4,5,6-tetrahydropyrazolo[3,4-e]indazole-3-carboxylate to give the desired product as a white solid (62% yield). FABHRMS m/z 311.0807 (M+H, C₁₂H₁₅N₄O₄S requires 311.0809).

Anal. Calcd for C₁₂H₁₄N₄O₄S (1H₂O): C, 43.90; H, 4.91; N, 17.06. Found: C, 43.98; H, 4.62; N, 17.02.

### EXAMPLE 195

This example illustrates the production of 8-(methylsulfonyl)-2,4,5,6-tetrahydro-pyrazolo[3,4-e]indazole-3-carboxylic acid:

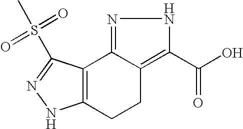

Ethyl 8-(methylsulfonyl)-2,4,5,6-tetrahydropyrazolo[3,4-e]indazole-3-carboxylate was reacted according to the procedure for the production of 7-hydroxy-4,5-dihydro-2H-benzo[g]indazole-3-carboxylic acid to give the desired product as a white solid (93% yield). FABHRMS m/z 283.0505 (M+H, C₁₀H₁₁N₄O₄S requires 283.0496).

Anal. Calcd for C₁₀H₁₀N₄O₄S (1.3H₂O): C, 39.29; H, 4.15; N, 18.33. Found: C, 39.16; H, 3.76; N, 18.29.

### EXAMPLE 196

This example illustrates the production of 8-(methylsulfonyl)-2,4,5,6-tetrahydro-pyrazolo[3,4-e]indazole-3-carboxamide.

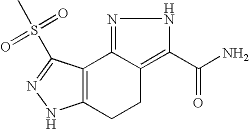

Ethyl 8-(methylsulfonyl)-2,4,5,6-tetrahydropyrazolo[3,4-e]indazole-3-carboxylate was reacted according to the procedure for the production of 8-hydroxy-4,5-dihydro-2H-benzo[g]indazole-3-carboxamide to give the desired product as a white solid (93% yield). FABHRMS m/z 282.0665 (M+H, C₁₀H₁₂N₅O₃S requires 282.0655).

Anal. Calcd for C₁₀H₁₁N₅O₃S(H₂O): C, 40.13; H, 4.38; N, 23.40. Found: C, 40.07; H, 4.20; N, 23.53.

### EXAMPLE 197

This example illustrates the production of 2-(3-aminopropyl)-2,6-dihydro-pyrazolo[3,4-e]indazole-3-carboxylic acid dihydrochloride:

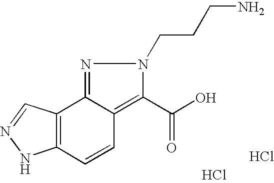

Ethyl 2-{3-[(tert-butoxycarbonyl)amino]propyl}-7-trityl-2,4,5,6-tetrahydropyrazolo[3,4-e]indazole-3-carboxylate (1.0 g, 0.0016 mol) prepared according to the procedure for the production of ethyl 2-(3-aminopropyl)-2,4,5,6-tetrahydropyrazolo[3,4-e]indazole-3-carboxylate dihydrochloride and DDQ (360 mg, 0.0016 mol) in dioxane (25 mL) were refluxed 4 hours. Contents were allowed to cool and the by-product was filtered. The filtrate was partitioned between EtOAc and water. The EtOAc layer was dried over MgSO₄ and concentrated in vacuo leaving a yellow oil, 900 mg. The oil was reacted according to the procedure for the production of 7-hydroxy-4,5-dihydro-2H-benzo[g]indazole-3-carboxylic acid to give 2-(3-aminopropyl)-7-trityl2,6-dihydropyrazolo[3,4-e]indazole-3-carboxylic acid as a white solid, 870 mg. The white solid was mixed with 4N HCl in dioxane (10 mL) and stirred overnight. Contents were filtered to give the desired product as a white solid, 417 mg (78% yield). FABHRMS m/z 260.1142 (M+H, C₁₂H₁₄N₅O₂ requires 260.1135). ¹H NMR (DMSO-d₆+5%TFA/300 MHz): 8.40 (s, 1H); 7.92 (d, 1H); 7.85 (br, 3H); 7.52 (d, 1H); 4.90 (t, 2H); 2.90-2.80 (m, 2H); 2.35-2.20 (m, 2H).

Anal. Calcd for C₁₂H₁₃N₅O₂ (2HCl, 2H₂O): C, 39.14; H, 5.20; N, 19.02. Found: C, 39.41; H, 4.63; N, 18.75.

### EXAMPLE 198

This example illustrates the production of 7,8,9,10-tetrahydro[1,4]-diazepino[1,2-b]pyrazolo[3,4-g]indazol-6(3H)-one:

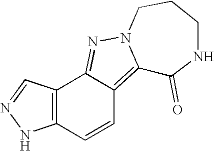

Ethyl 2-{3-[(tert-butoxycarbonyl)amino]propyl}-7-trityl-2,4,5,6-tetrahydropyrazolo-[3,4-e]indazole-3-carboxylate (2.0 g, 0.0032 mol) prepared according to the procedure for the production of ethyl 2-(3-aminopropyl)-2,4,5,6-tetrahydropyrazolo[3,4-e]indazole-3-carboxylate dihydrochloride and DDQ (720 mg, 0.0032 mol) in dioxane (25 mL) were refluxed 4 hours. Contents were allowed to cool and the by-product was filtered. The filtrate was partitioned between EtOAc and water. The EtOAc layer was dried over MgSO₄ and concentrated in vacuo leaving a yellow oil, 1.58 g. The yellow oil was mixed with 4N HCl in dioxane (25 mL) and stirred overnight. Contents were filtered to give a white solid, 530 mg, which is ethyl 2-(3-aminopropyl)-2,4,5,6-tetrahydropyrazolo[3,4-e]indazole-3-carboxylate dihydrochloride. The white solid was mixed with conc. ammonium hydroxide (30 mL) and methanol (15 mL) and stirred in a stoppered flask overnight. Contents were poured into a beaker to allow volatiles to evaporate and then filtered to give the desired product as a white solid, 384 mg (50% yield). FABHRMS m/z 242.1047 (M+H, C₁₂H₁₂N₅O requires 242.1036). ¹H NMR (DMSO-d₆+5%TFA/300 MHz): 8.40 (br, 1H), 8.35 (s, 1H); 7.80 (d, 1H); 7.40 (d, 1H); 4.73 (t, 2H); 3.30-3.20 (m, 2H); 2.35-2.20 (m, 2H).

Anal. Calcd for C₁₂H₁₁N₅O (0.3H₂O): C, 58.43; H, 4.74; N, 28.39. Found: C, 58.05; H, 4.56; N, 28.59.

### EXAMPLE 199

This example illustrates the production of 2-(3-aminopropyl)-8-(methylthio)-2,4,5,6-tetrahydropyrazolo[3,4-e]indazole-3-carboxylic acid dihydrochloride:

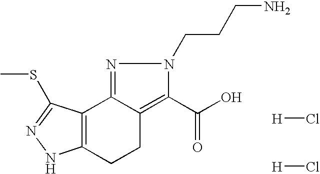

Ethyl 2-{3-[(tert-butoxycarbonyl)amino]propyl}-6-[(4-methylphenyl)sulfonyl]-8-(methylthio)-2,4,5,6-tetra hydropyrazolo[3,4-e]indazole-3-carboxylate (260 mg, 0.44 mmol) and 6N HCl (8 mL) were refluxed overnight. Contents were evaporated and the residue was triturated with EtOAc to give the desired product as a white solid, 70 mg (42% yield). FABHRMS m/z 308.1184 (M+H, C₁₃H₁₈N₅O₂S requires 308.1176). ¹H NMR (DMSO-d₆+5%TFA/300 MHz): 7.80 (br, 3H); 4.60-4.50 (m, 2H); 3.10-3.00 (m, 2H); 2.90-2.80 (m, 4H); 2.55 (s, 3H); 2.10-2.00 (m, 2H).

Anal. Calcd for C₁₇H₁₇N₅O₂S (2HCl, 1H₂O): C, 39.20; H, 5.31; N, 17.58. Found: C, 39.23; H, 5.03; N, 17.24.

### EXAMPLE 200

This example illustrates the production of 3-(allylthio)-1-[(4-methylphenyl)-sulfonyl]-1,5,6,7-tetrahydro-4H-indazol-4-one:

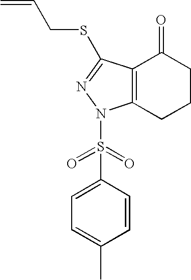

2-[bis(allylthio)methylene]cyclohexane-1,3-dione was prepared according to the procedure of 2-[bis(benzylthio)methylene]cyclohexane-1,3-dione, using allyl bromide (48% yield) and reacted according to the procedure for the production of 3-(benzylthio)-1-[(4-methylphenyl)sulfonyl]-1,5,6,7-tetrahydro-4H-indazol-4-one, to give the desired product after silica gel chromatography, eluting with 25% EtOAc/hexanes, as a white solid (41% yield). FABHRMS m/z 363.0843 (M+H, C₁₇H₁₉N₂O₃S₂ requires 363.0832). ¹H NMR (CDCl₃/300 MHz): 7.95 (d, 2H); 7.39 (d, 2H); 6.00-5.85 (m, 1H); 5.27 (d, 1H); 5.10 (d, 1H); 3.80 (d, 2H); 3.25 (t, 2H); 2.55-2.40 (m, 5H); 2.25-2.15 (m, 2H).

Anal. Calcd for C₁₅H₁₈N₂O₃S₂: C, 56.33; H, 5.01; N, 7.73. Found: C, 56.12; H, 4.57; N, 7.52.

### EXAMPLE 201

This example illustrates the production of ethyl {3-(allylthio)-1-[(4-methylphenyl) sulfonyl]-4-oxo-4,5,6,7-tetrahydro-1H-indazol-5-yl}(oxo)acetate:

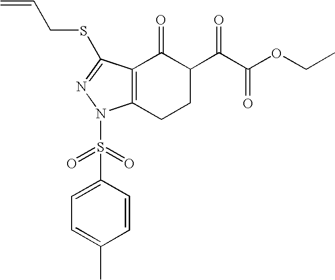

3-(allylthio)-1-[(4-methylphenyl)sulfonyl]-1,5,6,7-tetrahydro-4H-indazol-4-one was reacted according to the procedure for the production of ethyl (7-hydroxy-1-oxo-1,2,3,4-tetrahydronaphthalen-2-yl)(oxo)acetate to give the desired as a yellow solid (34% yield). FABHRMS m/z 463.0999 (M+H, C₂₁H₂₃N₂O₆S₂ requires 463.0992). OH NMR (CDCl₃/300 MHz): 7.90 (d, 2H); 7.40 (d, 2H); 6.00-5.85 (m, 1H); 5.30 (d, 1H); 5.10 (d, 1H); 4.35-4.24 (m, 2H); 3.80-3.75 (m, 2H) 3.35-3.20 (m, 4H); 2.50 (s, 3H); 1.40 (t, 3H).

Anal. Calcd for C₂₁H₂₃N₂O₆S₂: C, 54.53; H, 4.79; N, 6.06. Found: C, 54.64; H, 4.55; N, 5.69.

### EXAMPLE 202

This example illustrates the production of ethyl 8-(allylthio)-6-[(4-methylphenyl) sulfonyl]-2,4,5,6-tetrahydropyrazolo[3,4-e]indazole-3-carboxylate:

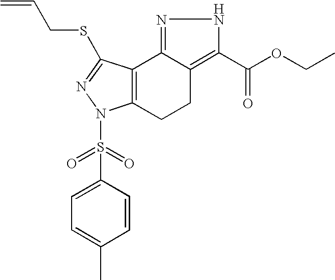

Ethyl {3-(allylthio)-1-[(4-methylphenyl)sulfonyl]-4-oxo-4,5,6,7-tetrahydro-1H-indazol-5-yl}(oxo)acetate was reacted according to the procedure for the production of ethyl 8-hydroxy-4,5-dihydro-2H-benzo[g]indazole-3-carboxylate, to give the desired product, after EtOAc recrystallization as yellow crystals (68% yield). FABHRMS m/z 459.1127 (M+H, C₂₁H₂₃N₄O₄S₂ requires 459.1155). ¹H NMR (CDCl₃/300 MHz): 7.85 (d, 2H); 7.35 (d, 2H); 6.00-5.85 (m, 1H); 5.25 (d, 2H); 5.05 (d, 2H); 4.40 (q, 2H); 3.80 (d, 2H); 3.40 (t, 2H); 3.20 (t, 2H); 2.45 (s, 3H).

Anal. Calcd for C₂₁H₂₂N₄O₄S₂: C, 55.00; H, 4.84; N, 12.22. Found: C, 54.93; H, 4.55; N, 12.13.

### EXAMPLE 203

This example illustrates the production of ethyl 8-(allylthio)-2-(3-t-butoxycarbonyl-aminopropyl)-6-[(4-methylphenyl)sulfonyl]-2,4,5,6-tetrahydropyrazolo[3,4-e]indazole-3-carboxylate:

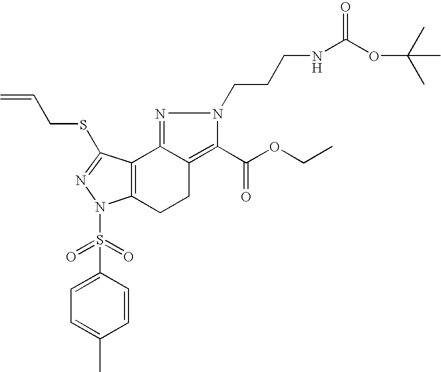

Ethyl 8-(allylthio)-6-[(4-methylphenyl)sulfonyl]-2,4,5,6-tetrahydropyrazolo-[3,4-e]indazole-3-carboxylate was reacted according to the procedure for the production of ethyl 2-(3-aminopropyl)-2,4,5,6-tetrahydropyrazolo[3,4-e]indazole-3-carboxylate dihydrochloride to give the desired product after silica gel chromatography eluting with 25% EtOAc/hexanes, as a white solid (70% yield). FABHRMS m/z 616.2264 (M+H, C₂₉H₃₈N₅O₆S₂ requires 616.2258). ¹H NMR (DMSO-d₆+TFA/300 M/Hz): 7.82 (d, 2H); 7.42 (d, 2H); 6.78 (br, 1H); 5.85-5.75 (m, 1H); 5.20 (d, 1H); 5.00 (d, 1H); 4.40 (t, 2H); 4.30 (q, 2H); 3.78 (d, 2H); 3.40-3.25 (m, 2H); 3.15-3.00 (m, 1H); 2.95-2.85 (m, 2H); 2.40 (s, 3H); 1.85-1.75 (m, 2H); 1.35 (s, 9H); 1.33 (t, 3H).

Anal. Calcd for C₂₉H₃₇N₅O₆S₂: C, 56.57; H. 6.06; N. 11.37. Found: C, 56.55; H. 6.21; N. 11.27.

### EXAMPLE 204

This example illustrates the production of 8-(allylthio)-2-(3-aminopropyl)-2,4,5,6-tetrahydropyrazolo[3,4-e]indazole-3-carboxylic acid dihydrochloride:

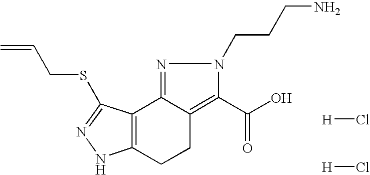

Ethyl 2-{3-[(tert-butoxycarbonyl)amino]propyl}-6-[(4-methylphenyl)sulfonyl]-8-(allylthio)-2,4,5,6-tetrahydropyrazolo[3,4-e]indazole-3-carboxylate was reacted according to the procedure for the production of 7-hydroxy-4,5-dihydro-2H-benzo[g]indazole-3-carboxylic acid and after treatment with 4N HCl in dioxane, was filtered to give the desired product as a white solid (33% yield). FABHRMS m/z 334.1336 (M+H, C₁₅H₂₀N₅O₂S requires 334.1332). ¹H NMR (DMSO-d₆+TFA/300 MHz): 7.80 (br, 3H); 5.90-5.80 (m, 1H); 5.10-4.95 (m, 2H); 4.60-4.50 (m, 2H); 3.80-3.70 (m, 2H); 3.10-3.00 (m, 2H); 2.90-2.80 (m, 4H); 2.10-2.00 (m, 2H).

Anal. Calcd for C₁₅H₁₉N₅O₂S (2HCl): C, 44.34; H, 5.21; N, 17.24. Found: C, 44.37; H, 4.89; N, 17.02.

### EXAMPLE 205

This example illustrates the production of ethyl 2-(3-aminopropyl)-8-(allylthio)-2,4,5,6-tetrahydropyrazolo[3,4-e]indazole-3-carboxylate dihydrochloride:

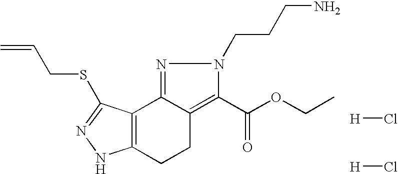

Ethyl 2-{3-[(tert-butoxycarbonyl)amino]propyl}-6-[(4-methylphenyl)sulfonyl]-8-(allylthio)-2,4,5,6-tetrahydropyrazolo[3,4-e]indazole-3-carboxylate (1.3 g, 0.002 mol) and 4 N HCl in dioxane (10 mL) were stirred overnight. Contents were diluted with EtOAc and filtered to give the desired as a white solid, 870 mg (95% yield). FABHRMS m/z 362.1632 (M+H, C₁₇H₂₄N₅O₂S requires 362.1645). ¹H NMR (DMSO-d₆+TFA/300 MHz): 7.80 (br s, 3H); 6.00-5.80 (m, 1H); 5.10-4.95 (m, 2H); 4.60-4.50 (m, 2H); 4.35 (q, 2H); 3.78 (d, 2H); 3.08-3.00 (m, 2H); 2.90-2.80 (m, 4H); 2.20-2.00 (m, 2H); 1.36 (t, 3H).

Anal. Calcd for C₁₇H₂₃N₅O₂S (2 HCl): C, 47.00; H, 5.80; N, 16.12. Found: C, 47.28; H, 5.52; N, 15.78.

### EXAMPLE 206

This example illustrates the production of 1-(allylthio)-4,5,7,8,9,10-hexahydro[1,4]diazepino[1,2-b]pyrazolo[3,4-g]indazol-6(3H)-one:

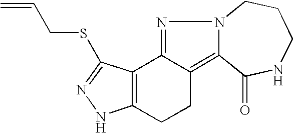

Ethyl 2-(3-aminopropyl)-8-(allylthio)-2,4,5,6-tetrahydropyrazolo[3,4-e]indazole-3-carboxylate dihydrochloride (800 mg, 0.002 mol), conc ammonium hydroxide (30 mL) and methanol (15 mL) were stirred in a stoppered flask overnight. Contents were poured into a beaker to let the volatiles evaporate and filtered to give the desired product as a pale green solid, 283 mg (49% yield). FABHRMS m/z 316.1231 (M+H, C₁₅H₁₈N₅OS requires 316.1227). ¹H NMR (DMSO-d₆+TFA/300 MHz): 8.20 (br s, 1H); 6.00-5.80 (m, 1H); 5.15-4.90 (m, 2H); 4.40 (t, 2H); 3.80 (d, 2H); 3.22-3.10 (m, 2H); 2.95-2.80 (m, 4H); 2.20-2.00 (m, 2H).

Anal. Calcd for C₁₅H₁₇N₅OS (0.5H₂O): C, 55.54; H, 5.59; N, 21.59. Found: C, 55.28; H, 5.25; N, 21.36.

### EXAMPLE 207

This example illustrates the production of 4,5,7,8,9,10-hexahydro[1,4-diazepino[1,2-b]pyrazolo[3,4-g]indazol-6(3H)-one:

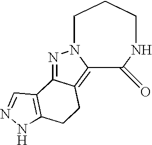

Ethyl 2-(3-aminopropyl)-2,4,5,6-tetrahydropyrazolo[3,4-e]indazole-3-carboxylate dihydrochloride (500 mg, 0.0014 mol), conc ammonium hydroxide (30 mL) and methanol (15 mL) were stirred in a stoppered flask overnight. Contents were poured into a beaker to let the volatiles evaporate and filtered to give the desired product as a light amber solid, 325 mg (97% yield). FABHRMS m/z 244.1204 (M+H, C₁₂H₁₄N₅O requires 244.1193). ¹H NMR (DMSO-d₆/300 MHz): 8.20 (br s, 1H); 7.82 (br s, 1H); 4.38 (t, 2H); 3.20 (br s, 2H); 2.98-2.70 (m, 4H); 2.20-2.00 (m, 2H).

Anal. Calcd for C₁₂H₁₃N₅O (0.6H₂O): C, 56.73; H, 5.63; N, 27.56. Found: C, 56.56; H, 5.82; N, 27.95.

### EXAMPLE 208

This example illustrates the production of ethyl 2-(3-{[2-(4-bromophenyl) ethyl]amino}propyl)-2,4,5,6-tetrahydropyrazolo[3,4-e]indazole-3-carboxylate:

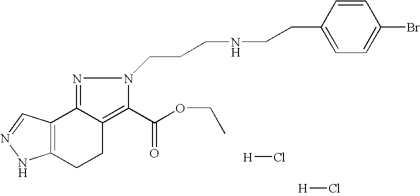

To 4-bromophenethylalcohol (29.5 g, 0.147 mol) and triethylamine (20.9 mL, 0.15 mol) in CH₂Cl₂ (250 ml) at 0° C. was added dropwise methansulfonylchloride (11.6 mL, 0.15 mol) in CH₂Cl₂ (50 mL). Contents were stirred overnight, Coming to room temperature, washed with water, dried over MgSO₄, and concentrated in vacuo leaving the mesylate intermediate as a white solid, 51.6 g. The mesylate intermediate (51.6 g, 0.185 mol), 3-amino-1-propanol (20.8 g, 0.28 mol) and potassium carbonate (38.7 g, 0.28 mol) in acetonitrile (500 mL) were refluxed for 8 hours. Contents were allowed to cool, quenched with water and extracted with EtOAc. The EtOAc layer was washed with brine, dried over MgSO₄, and concentrated in vacuo leaving 4-bromophenethylaminopropanol as a light yellow oil, which solidified, 40.1 g. 4-Bromophenethylaminopropanol (40.1 g, 0.16 mol) and di-t-butyl-dicarbonate (35.0 g, 0.16 mol) in CH₂Cl₂ (500 mL) were stirred overnight. Contents were washed with water, brine, dried over MgSO₄ and concentrated in vacuo leaving 4-bromophenethyl-Boc-aminopropanol as a light yellow oil, 53.7 g. To 4-bromophenethyl-boc-aminopropanol (51.8 g; 0.145 mol) and triethylamine (21 mL, 0.15 mol) in CH₂Cl₂ (500 mL) at 0° C. was added dropwise methansulfonylchloride (11.6 mL, 0.15 mol) in CH₂Cl₂ (50 mL). Contents were stirred 5 hours, Coming to room temperature, then washed with water, brine, dried over MgSO₄ and concentrated in vacuo leaving 3-[[2-(4-bromophenyl)ethyl](tert-butoxycarbonyl)amino]propyl methanesulfonate as a yellow oil, 48.0 g. To ethyl 6-trityl-2,4,5,6-tetrahydropyrazolo[3,4-e]indazole-3-carboxylate (11.9 g, 0.025 mol) in DMF (100 mL) at 0° C. was added dropwise lithium t-butoxide (1M in THF, 50 mL). Contents were stirred one hour at 0° C. and added 3-[[2-(4-bromophenyl)ethyl](tert-butoxycarbonyl)amino]propyl methanesulfonate (22.1 g, 0.05 mol) in DMF (25 mL) dropwise. Contents were stirred overnight, Coming to room temperature and partitioned between EtOAc and water. The EtOAc layer was washed with brine, dried over MgSO₄ and concentrated in vacuo leaving ethyl 2-(3-{[2-(4-bromophenyl)ethyl]amino}propyl)-7-trityl-2,4,5,6-tetra hydropyrazolo[3,4-e]indazole-3-carboxylate as an amber oil, 26.8 g. Ethyl 2-(3-{[2-(4-bromophenyl)ethyl]amino}propyl)-7-trityl-2,4,5,6-tetrahydropyrazolo[3,4-e]indazole-3-carboxylate (9.8 g, 0.014 mol) and 4N HCl in dioxane (100 mL) were stirred overnight. Contents were diluted with EtOAc and filtered to give the desired as a white solid, 3.6 g (55% yield). FABHRMS m/z 472.1342 (M+H, C₂₂H₂₇BrN₅O₂ requires 472.1343). ¹H NMR (DMSO-d₆+TFA/300 MHz): 9.00 (br, 2H); 8.00 (s, 1H); 7.50 (d, 2H); 7.20 (d, 2H); 4.60-4.50 (m, 2H); 4.35 (q, 2H); 3.20-2.90 (m, 10H); 2.20-2.10 (m, 2H); 1.38 (t, 3H).

Anal. Calcd for C₂₂H₂₆BrN₅O₂ (2HCl, H₂O): C, 46.99; H, 5.20; N, 12.45. Found: C, 46.93; H, 5.41; N, 12.37.

### EXAMPLE 209

This example illustrates the production of 2-(3-{[2-(4-bromophenyl)ethyl]amino}propyl)-2,4,5,6-tetrahydropyrazolo[3,4-e]indazole-3-carboxylic acid hydrochloride:

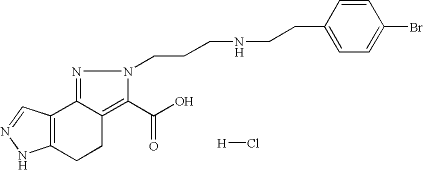

Ethyl 2-(3-{[2-(4-bromophenyl)ethyl]amino}propyl)-2,4,5,6-tetrahydropyrazolo[3,4-e]indazole-3-carboxylate, was reacted according to the procedure for the production of 7-hydroxy-4,5-dihydro-2H-benzo[g]indazole-3-carboxylic acid to give the desired product as a white solid (52% yield). FABHRMS m/z 444.1015 (M+H, C₂₀H₂₃BrN₅O₂ requires 444.1030). ¹H NMR (DMSO-d₆+TFA/300 MHz): 8.80 (br, 2H); 8.10 (s, 1H); 7.50 (d, 2H); 7.20 (d, 2H); 4.60-4.55 (m, 2H); 3.20-2.90 (m, 10H); 2.20-2.10 (m, 2H).

Anal. Calcd for C₂₀H₂₂BrN₅O₂(2HCl, 2H₂O): C, 43.42; H, 5.10; N, 12.66. Found: C, 43.06; H, 5.25; N, 12.68.

### EXAMPLE 210

This example illustrates the production of ethyl 2-{3-[(2-thien-3-ylethyl)amino]propyl}-2,4,5,6-tetrahydropyrazolo[3,4-e]indazole-3-carboxylate dihydrochloride:

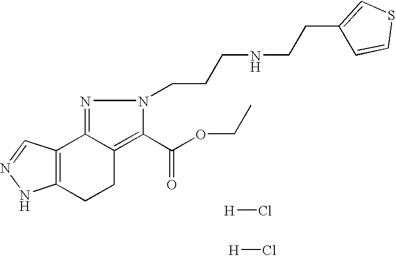

Ethyl 2-{3-[(2-thien-3-ylethyl)amino]propyl}-2,4,5,6-tetrahydropyrazolo[3,4-e]indazole-3-carboxylate dihydrochloride was prepared according to the procedure of ethyl 2-(3-{[2-(4-bromophenyl)ethyl]amino}propyl)-2,4,5,6-tetrahydropyrazolo[3,4-e]indazole-3-carboxylate, using 2-(3-theinyl)ethanol, to give the desired product as a white solid (45% yield). FABHRMS m/z 400.1797 (M+H, C₂₀H₂₆N₅O₂S requires 400.1802). ¹H NMR (DMSO-d₆+TFA/300 MHz): 8.80 (br, 2H); 8.10 (s, 1H); 7.50 (s, 1H); 7.30 (s, 1H); 7.00 (d, 1H); 4.60-4.55 (m, 2H); 4.30 (q, 2H); 3.25-2.90 (m, 10H) 2.20-2.10 (m, 2H); 1.35 (t, 3H).

Anal. Calcd for C₂₀H₂₅N₅O₂S (2HCl): C, 50.85; H, 5.76; N, 14.82. Found: C, 51.43; H, 6.04; N, 14.48.

### EXAMPLE 211

This example illustrates the production of 2-{3-[(2-thien-3-ylethyl)amino]propyl}-2,4,5,6-tetrahydropyrazolo[3,4-e]indazole-3-carboxylic acid hydrochloride:

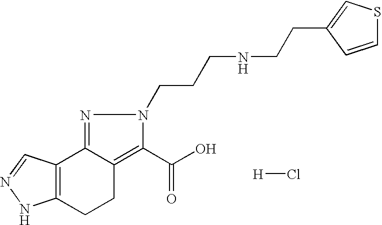

Ethyl 2-{3-[(2-thien-3-ylethyl)amino]propyl}-2,4,5,6-tetrahydropyrazolo[3,4-e]indazole-3-carboxylate dihydrochloride was reacted according to the procedure for the production of 7-hydroxy-4,5-dihydro-2H-benzo[g]indazole-3-carboxylic acid to give the desired as a white solid (28% yield). FABHRMS m/z 372.1451 (M+H, C₁₈H₂₂N₅O₂S requires 372.1489). ¹H NMR (DMSO-d₆+TFA/300 MHz): 8.55 (br, 2H); 8.10 (s, 1H); 7.48 (s, 1H); 7.30 (s, 1H); 7.00 (d, 1H); 4.60-4.50 (m, 2H); 3.30-2.90 (m, 10H); 2.20-2.05 (m, 2H).

### EXAMPLE 212

This example illustrates the production of 2-{3-[(2-thien-2-ylethyl)amino]-propyl}-2,4,5,6-tetrahydropyrazolo[3,4-e]indazole-3-carboxylic acid:

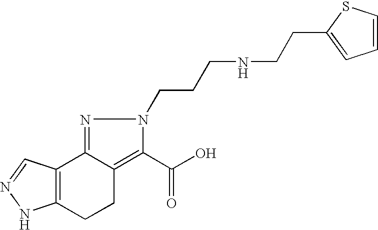

2-{3-[(2-thien-2-ylethyl)amino]propyl}-2,4,5,6-tetrahydropyrazolo[3,4-e]indazole-3-carboxylic acid was prepared according to the procedures of ethyl 2-(3-{[2-(4-bromophenyl)ethyl]amino}propyl)-2,4,5,6-tetrahydropyrazolo[3,4-e]indazole-3-carboxylate and 7-hydroxy-4,5-dihydro-2H-benzo[g]indazole-3-carboxylic acid to give the desired product as a white solid (73% yield). FABHRMS m/z 372.1466 (M+H, C₁₈H₂₂N₅O₂S requires 372.1489). ¹H NMR (DMSO-d₆+TFA/300 MHz): 8.55 (br, 2H); 7.95 (s, 1H); 7.40 (d, 2H); 7.00-6.95 (m, 2H); 4.60-4.50 (m, 2H); 3.30-2.90 (m, 10H); 2.20-2.00 (m, 2H).

Anal. Calcd for C₁₈H₂₁N₅O₂S (0.3H₂O): C, 57.37; H, 5.78; N, 18.58. Found: C, 57.38; H, 5.99; N, 18.55.

### EXAMPLE 213

This example illustrates the production of ethyl 2-{3-[(2-thien-2-ylethyl)amino]propyl}-2,6-dihydropyrazolo[3,4-e]indazole-3-carboxylate:

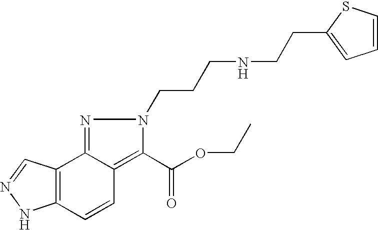

Ethyl 6-trityl-2,4,5,6-tetrahydropyrazolo[3,4-e]indazole-3-carboxylate was reacted with 3-[[2-(2-thienylethyl](tert-butoxycarbonyl)-amino]propyl methanesulfonate according to the procedure for the production of ethyl 2-(3-{[2-(4-bromophenyl)ethyl]amino}propyl)-2,4,5,6-tetrahydropyrazolo[3,4-e]indazole-3-carboxylate to give ethyl 2-(3-{[2-(2-thienyl)ethyl]amino}propyl)-7-trityl-2,4,5,6-tetrahydropyrazolo[3,4-e]indazole-3-carboxylate as a yellow oil. The oil (9.9 g, 0.013 mol) and DDQ (3.0 g, 0.013 mol) were refluxed in dioxane (100 mL) for 5 hours. Contents were allowed to cool and a by-product was filtered. The filtrate was concentrated in vacuo and the dark oily residue was filtered through a pad of silica gel, eluting with 25% EtOAc/hexanes to give a pale yellow oil, 4.1 g. The oil was mixed with 4N HCl in dioxane and stirred overnight and filtered to give the desired product as a white solid, 1.4 g (27% yield). FABHRMS m/z 398.1654 (M+H, C₂₀H₂₄N₅O₂S requires 398.1645). ¹H NMR (DMSO-d₆+TFA/300 MHz): 8.80 (br, 2H); 8.35 (s, 1H); 7.90 (d, 1H); 7.50 (d, 1H); 7.35 (d, 1H); 7.00-6.95 (m, 2H); 4.92 (t, 2H); 4.43 (q, 2H); 3.25-3.00 (m, 6H); 2.40-2.25 (m, 2H); 1.42 (t, 3H).

Anal. Calcd for C₂₀H₂₃N₅O₂S (2HCl): C, 51.07; H, 5.36; N, 14.89. Found: C, 51.29; H, 5.77; N, 14.78.

### EXAMPLE 214

This example illustrates the production of 2-{3-[(2-thien-2-ylethyl)amino]propyl}-2,6-dihydropyrazolo[3,4-e]indazole-3-carboxylic acid.

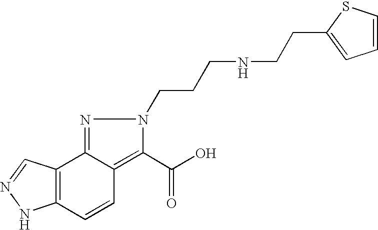

Ethyl 2-{3-[(2-thien-2-ylethyl)amino]propyl}-2,6-dihydropyrazolo[3,4-e]indazole-3-carboxylate was reacted according to the procedure for the production of 7-hydroxy-4,5-dihydro-2H-benzo[g]indazole-3-carboxylic acid to give the desired product as a white solid (84% yield). FABHRMS m/z 370.1327 (M+H, C₁₈H₂₀N₅O₂S requires 370.1332). ¹H NMR (DMSO-d₆+TFA/300 MHz): 8.60 (br, 2H); 8.35 (s, 1H); 7.90 (d, 1H); 7.50 (d, 1H); 7.40 (s, 1H); 7.00-6.95 (m, 2H); 5.00-4.90 (m, 2H); 3.30-3.00 (m, 6H); 2.40-2.25 (m, 2H).

Anal. Calcd for C₁₈H₁₉N₅O₂S (0.25H₂O): C, 57.82; H, 5.26; N, 18.73. Found: C, 57.78; H, 5.48; N, 18.87.

### EXAMPLE 215

This example illustrates the production of ethyl 1-{1-[(benzyloxy) carbonyl]piperidin-4-yl}-7-trityl-1,4,5,6-tetrahydropyrazolo[3,4-e]indazole-3-carboxylate.

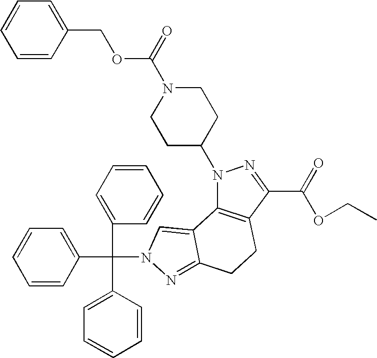

Benzyl 4-oxopiperidine carboxylate (29.3 g, 0.126 mol) and t-butyl carbazate (16.6 g, 0.126 mol) in ethanol (180 mL) were stirred overnight. Contents were concentrated in vacuo leaving benzyl 4-[2-t-butoxycarbonyl)hydrazono]-piperidine-1-carboxylate as a colorless oil, 49.3 g. To benzyl 4-[2-t-butoxy-carbonyl)hydrazono]piperidine-1-carboxylate (48 g, 0.14 mole) in THF (150 mL) at 0° C. was added BH₃-THF (1M, 200 mL) over 30 minutes. Contents were stirred 2 hours, Coming to room temperature and carefully quenched with methanol (50 mL). Contents were concentrated in vacuo, diluted with EtOAc, washed with 50% aqueous sodium bicarbonate (200 mL), brine, dried over MgSO₄, concentrated in vacuo to about 50 mL, and filtered to give benzyl 4-[2-t-butoxycarbonyl)hydrazino]piperidine-1-carboxylate as a white solid, 36.2 g. Benzyl 4-[2-t-butoxycarbonyl)hydrazono]piperidine-1-carboxylate (2.3 g, 0.0066 mol) and 4N HCl in dioxane (5 mL) were stirred for a half hour and concentrated in vacuo leaving a white foam. The white foam was dissolved in methanol (10 mL) and 0.5M sodium methoxide (13 mL) was added dropwise. Contents were stirred a half hour and ethyl oxo(4-oxo-1-trityl-4,5,6,7-tetrahydro-1H-indazol-5-yl)acetate- or ethyl oxo(4-oxo-2-trityl-4,5,6,7-tetrahydro-2H-indazol-5-yl)acetate or ethyl oxo(4-oxo-2-trityl-4,5,6,7-tetrahydro-2H-indazol-5-yl)acetate (3.15 g, 0.0066 mol) in CH₂Cl₂/methanol (1:1, 10 mL) was added dropwise. Contents were stirred 72 hours and concentrated in vacuo leaving a foam, 5.2 g. The foam was purified by silica gel chromatography, eluting with 15% EtOAc/hexanes. Fractions 5-11 contained a light amber foam, 786 mg used in 2-piperidin-4-yl-2,4,5,6-tetrahydropyrazolo[3,4-e]indazole-3-carboxylic acid hydrochloride. Fractions 14-28 were concentrated in vacuo leaving a light amber foam, 2.5 g, which crystallized from methanol to give the desired as a white solid, 1.7 g (74% yield). FABHRMS m/z 692.3237 (M+H, C₄₃H₄₂N₅O₄ requires 692.3231). ¹H NMR (CDCl₃/300 MHz): 7.45-7.20 (m, 21H); 5.19 (s, 2H); 4.45 (q, 2H); 4.40-4.30 (m, 2H); 4.05 (m, 1H); 3.25-3.15 (m, 2H); 3.05-2.95 (m, 2H); 2.80-2.55 (m, 2H); 2.35-2.10 (m, 2H); 2.00-1.90 (m, 2H); 1.42 (t, 3H).

Anal. Calcd for C₄₃H₄₁N₅O₄: C, 74.65; H, 5.97; N, 10.12. Found: C, 74.39; H, 5.69; N, 9.99.

### EXAMPLE 216

This example illustrates the production of 2-piperidin-4-yl-2,4,5,6-tetrahydropyrazolo[3,4-e]indazole-3-carboxylic acid hydrochloride.

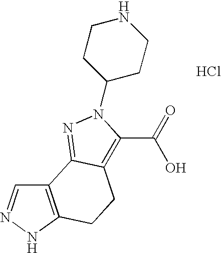

Ethyl 2-{1-[(benzyloxy)carbonyl]piperidin-4-yl}-7-trityl-1,4,5,6-tetrahydropyrazolo[3,4-e]indazole-3-carboxylate was obtained in ethyl 1-{1-[(benzyloxy)carbonyl]piperidin-4-yl}-6-trityl-1,4,5,6-tetrahydropyrazolo[3,4-e]indazole-3-carboxylate in Fractions 5-11 as a light amber foam (17% yield). The foam (786 mg), palladium/carbon (10%, 93 mg) and methanol (20 mL) were shaken at 55 psi H₂ on a Parr hydrogenator for 45 minutes. Contents were filtered through clay and the filtrate was concentrated in vacuo. The residue (670 mg), 2.5N sodium hydroxide (4 mL), and methanol (20 mL) were refluxed for 2 hours. Contents were diluted with water (20 mL) and extracted with EtOAc. The aqueous layer was made acidic with 1N HCl to pH 2 and extracted with EtOAc. The EtOAc layer was dried over MgSO₄ and concentrated in vacuo leaving a yellow solid (129 mg). The solid was mixed with 4N HCl in dioxane for 3 hours, concentrated in vacuo and the residue triturated with EtOAc and filtered to give the desired product as a light tan solid, 18 mg (6% yield). FABHRMS m/z 288.1489 (M+H, C₄₃H₄₂N₅O₄ requires 288.1455). ¹H NMR (DMSO-d₆+5%TFA/300 MHz): 8.80 (brd, 1H); 8.45 (br d, 1H); 8.02 (s, 1H); 5.42-5.35 (m, 1H); 3.50-3.40 (m, 2H); 3.20-2.80 (m, 6H); 2.35-2.05 (m, 4H).

### EXAMPLE 217

This example illustrates the production of 1-{1-[(benzyloxy)carbonyl]-piperidin-4-yl}-7-trityl-1,4,5,6-tetrahydropyrazolo[3,4-e]indazole-3-carboxylic acid.

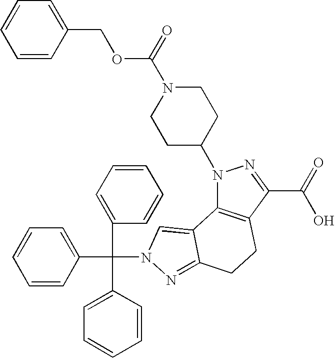

Ethyl 1-{1-[(benzyloxy)carbonyl]piperidin-4-yl}-6-trityl-1,4,5,6-tetrahydropyrazolo[3,4-e]indazole-3-carboxylate was reacted according to the procedure for the production of 7-hydroxy-4,5-dihydro-2H-benzo[g]indazole-3-carboxylic acid to give the desired as a white solid (77% yield). FABHRMS m/z 664.2947 (M+H, C₄₁H₃₈N₅O₄ requires 664.2918). ¹H NMR (DMSO-d₆+5%TFA/300 MHz): 7.62 (s, 1H); 7.40-7.10 (m, 20H); 5.12 (s, 2H); 4.47-4.37 (m, 1H); 4.08 (d, 2H); 3.10-3.00 (m, 2H); 2.85-2.80 (m, 4H); 2.00-1.80 (m, 4H).

Anal. Calcd for C₄₁H₃₇N₅O₄(1.8H₂O): C, 70.73; H, 5.88; N, 10.06. Found: C, 70.70; H, 5.71; N, 9.68.

### EXAMPLE 218

This example illustrates the production of 1-piperidin-4-yl-7-trityl-1,4,5,6-tetrahydropyrazolo[3,4-e]indazole-3-carboxylic acid.

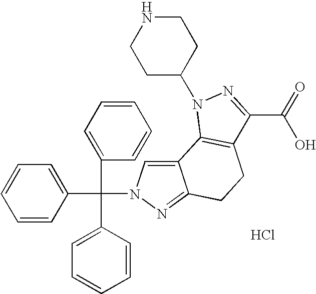

Ethyl 1-{1-[(benzyloxy)carbonyl]piperidin-4-yl}-6-trityl-1,4,5,6-tetrahydropyrazolo[3,4-e]indazole-3-carboxylate (1.4 g, 0.002 mol), 10% palladium/carbon (300 mg) and DMF (50 mL) were shaken at 55 psi H₂ on a Parr hydrogenator for 45 minutes. Contents were filtered through clay and the filtrate was concentrated in vacuo leaving an oil (1.4 g). The oil, 2.5N sodium hydroxide (4 mL), and methanol (15 mL) were refluxed 3 hours. Contents were concentrated in vacuo to about half and the residue made acidic with 1N HCl to pH 2. Contents were filtered to give the desired product as a white solid, 1.0 g (93% yield). FABHRMS m/z 530.2573 (M+H, C₃₃H₃₂N₅O₂ requires 530.2551).

Anal. Calcd for C₃₃H₃₁N₅O₂(2HCl): C, 65.78; H, 5.52; N, 11.62. Found: C, 65.81; H, 5.81; N, 11.53.

### EXAMPLE 219

This example illustrates the production of 1-piperidin-4-yl-1,4,5,6-tetrahydropyrazolo[3,4-e]indazole-3-carboxylic acid.

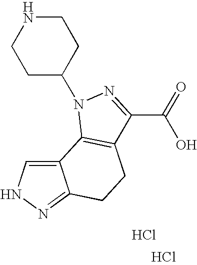

1-piperidin-4-yl-6-trityl-1,4,5,6-tetrahydropyrazolo[3,4-e]indazole-3-carboxylic acid hydrochloride (841 mg, 0.0016 mol) and 4N HCl in dioxane (20 mL) were stirred overnight and filtered to give the desired product as a white solid, 578 mg (96% yield). FABHRMS m/z 288.1437 (M+H, C₁₄H₁₈N₅O₂ requires 288.1455).

Anal. Calcd for C₁₄H₁₇N₅O₂(2HCl, H₂O): C, 44.45; H, 5.60; N, 18.51. Found: C, 44.72; H, 5.31; N, 18.28.

### EXAMPLE 220

This example illustrates the production of ethyl 7-oxo-4,5,6,7-tetrahydro-1H-indazole-3-carboxylate.

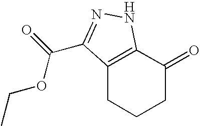

Acetyl chloride (50 mL) was added dropwise to ethanol (250 mL) at 0° C. Contents were stirred for 2 hours. 1,2-cyclohexanedione (26.5 g, 0.24 mol) was added and stirred 2 hours. Contents were concentrated in vacuo to about half and diluted with ether and water. Saturated aqueous sodium bicarbonate was added to pH 7. The ether layer was dried over MgSO₄ and concentrated in vacuo leaving 2-ethoxy-2-cyclohexene-1-one as an oil, 24.0 g (71% yield). 2-Ethoxy-2-cyclohexene-1-one was reacted according to the procedure for the production of ethyl (7-hydroxy-1-oxo-1,2,3,4-tetrahydronaphthalen-2-yl)(oxo)acetate to give the diketo-ester as a yellow oil (32% yield). The diketo-ester was reacted according to the procedure for the production of ethyl 8-hydroxy-4,5-dihydro-2H-benzo[g]indazole-3-carboxylate, using hydrazine monohydro-chloride to give the desired product as a yellow solid (95% yield). ¹H NMR (CDCl₃/300 MHz): 4.40 (q, 2H); 3.05 (t, 2H); 2.61 (t, 2H); 2.18 (quintet, 2H); 1.40 (t, 3H).

### EXAMPLE 221

This example illustrates the production of ethyl (6E)-6-[(dimethylamino)-methylene]-7-oxo-2-[3-(tritylamino)propyl]-4,5,6,7-tetrahydro-2H-indazole-3-carboxylate.

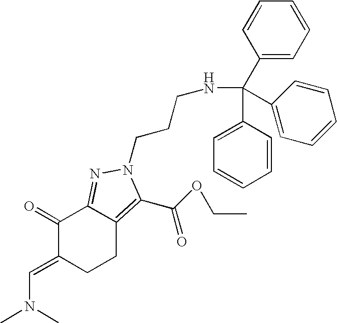

To 3-aminopropyl bromide hydrobromide (50.0 g, 0.228 mol) and trityl chloride (66.9 g, 0.24 mol) in CH₂Cl₂ (600 mL) was added dropwise triethylamine (70 mL, 0.5 mol). Contents were stirred overnight, washed with water, brine, dried over MgSO₄ and concentrated in vacuo leaving a white solid. The solid was triturated with EtOAc and filtered to give 3-(N-tritylamino)propylbromide as a white solid (91% yield). To ethyl 7-oxo-4,5,6,7-tetrahydro-1H-indazole-3-carboxylate (24.9 g, 0.12 mol) in THF (700 mL) at 0° C. was added dropwise potassium t-butoxide (1M in THF, 0.13 mol) and contents were stirred 3 hours. 3-(N-tritylamino)propyl bromide (49.4 g, 0.13 mol) in DMF (100 mL) was added and refluxed 3 hours. Contents were partitioned between EtOAc and water. The EtOAc layer was washed with brine, dried over MgSO₄ and concentrated in vacuo leaving an oil. The oil was purified by silica gel chromatography eluting first with 10% EtOAc/hexanes, then 25% EtOAc/hexanes to give ethyl 7-oxo-2-[3-(tritylamino)propyl]-4,5,6,7-tetrahydro-2H-indazole-3-carboxylate as an oil (19% yield). To ethyl 7-oxo-2-[3-(tritylamino)propyl]-4,5,6,7-tetrahydro-2H-indazole-3-carboxylate (12.3 g, 0.024 mol) in THF (500 mL) was added dropwise Bredereck's reagent (10.0 mL, 0.048 mol) in THF (10 mL). Contents were refluxed for 5 hours, allowed to cool and concentrated in vacuo leaving a dark oil. The oil was purified by silica gel chromatography eluting with 50% EtOAc/hexanes to give the desired product as a yellow solid, 6.5 g (48% yield). ¹H NMR (CDCl₃/300 MHz): 7.65 (s, 1H); 7.55-7.10 (m, 16H); 4.80-4.70 (m, 2H); 4.30 (q, 2H); 3.12 (s, 6H); 3.00-2.85 (m, 4H); 2.20-2.00 (m, 4H); 1.35 (t, 3H).

Anal. Calcd for C₃₅H₃₈N₄O₃(0.2H₂O): C, 74.23; H, 6.83; N, 9.89. Found: C, 74.16; H, 6.63; N, 9.77.

### EXAMPLE 222

This example illustrates the production of 2-(3-aminopropyl)-8-(4-methoxyphenyl)-2,4,5,8-tetrahydropyrazolo[4,3-g]indazole-3-carboxylic acid hydrochloride.

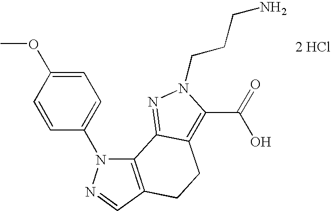

To 4-methoxyphenylhydrazine hydrochloride (786 mg, 0.0045 mol) in methanol (10 mL) was added dropwise 0.5M sodium methoxide (9 mL). Contents were stirred a half hour and added to ethyl (6E)-6-[(dimethylamino)methylene]-7-oxo-2-[3-(tritylamino)propyl]-4,5,6,7-tetrahydro-2H-indazole-3-carboxylate (2.3 g, 0.004 mol) in methanol (25 mL). Contents were refluxed 2 hours, allowed to cool and concentrated in vacuo leaving a red oil. The oil was purified by silica gel chromatography eluting with 25% EtOAc/hexanes, to give ethyl 2-(3-tritylaminopropyl)-8-(4-methoxyphenyl)-2,4,5,8-tetrahydropyrazolo[4,3-g]indazole-3-carboxylate as a red oil (1.5 g). The red oil, 2.5N sodium hydroxide (5 mL), water (5 mL) and methanol (20 mL) were refluxed 2 hours. Contents were partitioned between EtOAc and water. The water layer was made acidic to pH 2.5 with 1N HCl and filtered to give 2-(3-tritylaminopropyl)-8-(4-methoxyphenyl)-2,4,5,8-tetrahydropyrazolo[4,3-g]indazole-3-carboxylic acid as a light tan solid (702 mg). 2-(3-tritylaminopropyl)-8-(4-methoxyphenyl)-2,4,5,8-tetrahydropyrazolo[4,3-g]indazole-3-carboxylic acid (563 mg) and TFA (2 mL) in CH₂Cl₂ (5 mL) were stirred overnight. Contents were concentrated in vacuo and the residue mixed with 4N HCl in dioxane (5 mL) for 3 hours. Contents were filtered and the amber solid (315 mg) was triturated with acetonitrile/methanol to give the desired product as a light amber solid, 263 mg (15% yield. FABHRMS m/z 368.1696 (M+H, C₁₉H₂₂N₅O₃ requires 368.1717). ¹H NMR (DMSO-d₆+TFA/300 MHz): 7.90 (br, 2H); 7.60 (d, 2H); 7.55 (s, 1H); 7.05 (d, 2H); 4.45 (t, 2H); 3.80 (s, 3H); 3.05-2.95 (m, 2H); 2.80-2.70 (m, 4H); 2.10-1.95 (m, 2H).

Anal. Calcd for C₁₉H₂₁N₅O₃(1.2H₂O): C, 49.40; H, 5.54; N, 15.16. Found: C, 49.70; H, 5.67; N, 14.81.

### EXAMPLE 223

This example illustrates the production of ethyl 7-(acetylamino)-1-[4-(aminosulfonyl)phenyl]-4,5-dihydro-1H-benzo[g]indazole-3-carboxylate.

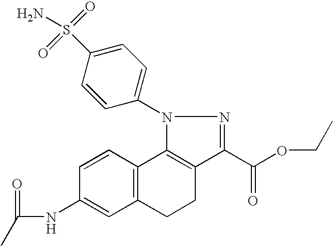

FABHRMS m/z 455.1393 (M+H, C₂₂H₂₃N₄O₅S requires 455.1389).

### EXAMPLE 224

This example illustrates the production of methyl 7-amino-1-[4-(aminosulfonyl) phenyl]-4,5-dihydro-1H-benzo[g]indazole-3-carboxylate hydrochloride.

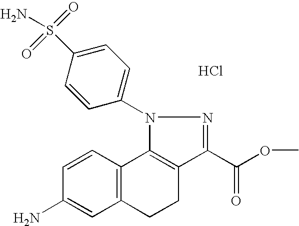

FABHRMS m/z 399.1150 (M+H, C₁₉H₁₉N₄O₄S requires 399.1127).

### EXAMPLE 225

This example illustrates the production of ethyl 6-methyl-2,4,5,6-tetrahydropyrazolo[3,4-e]indazole-3-carboxylate.

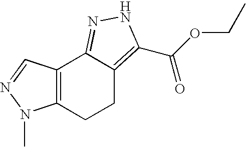

FABHRMS m/z 247.1208 (M+H, C₁₂H₁₅N₄O₂ requires 247.1195).

### EXAMPLE 226

This example illustrates the production of 7-amino-1-[4-(aminosulfonyl)phenyl]-4,5-dihydro-1H-benzo[g]indazole-3-carboxamide.

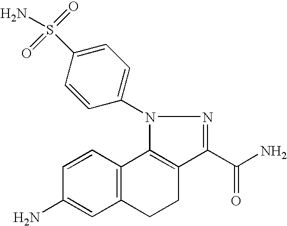

FABHRMS m/z 384.1135 (M+H, C₁₈H₁₈N₅O₃S requires 384.1130).

### EXAMPLE 227

This example illustrates the production of 8-amino-1-[4-(aminosulfonyl)phenyl]-4,5-dihydro-1H-benzo[g]indazole-3-carboxylic acid.

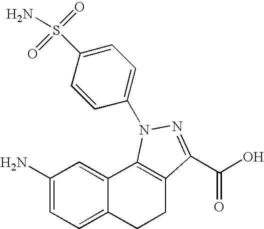

FABHRMS m/z 385.0999 (M+H, C₁₈H₁₇N₄O₄S requires 385.0965).

### EXAMPLE 228

This example illustrates the production of 1-[4-(aminosulfonyl)phenyl]-6-benzyl-N-methyl-1,4,5,6-tetrahydropyrazolo[3,4-e]indazole-3-carboxamide.

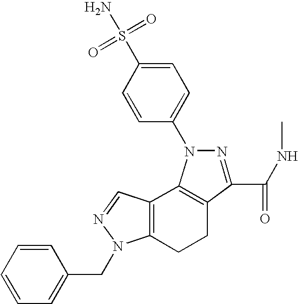

FABHRMS m/z 463.1566 (M+H, C₂₃H₂₃N₆O₃S requires 463.1552).

### EXAMPLE 229

This example illustrates the production of 8-amino-1-[4-(aminosulfonyl)phenyl]-N-methyl-4,5-dihydro-1H-benzo[g]indazole-3-carboxamide.

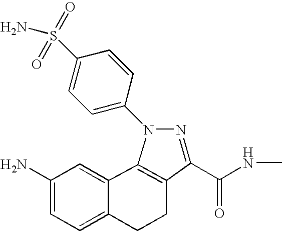

FABHRMS m/z 398.1266 (M+H, C₁₉H₂₀N₅O₃S requires 398.1287).

### EXAMPLE 230

This example illustrates the production of ethyl 6-phenyl-2,4,5,6-tetrahydro-pyrazolo[3,4-e]indazole-3-carboxylate.

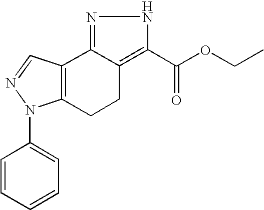

FABHRMS m/z 309.1330 (M+H, C₁₇H₁₇N₄O₂ requires 309.1352).

### EXAMPLE 231

This example illustrates the production of 1-[4-(aminosulfonyl)phenyl]-N-methyl-1,4,5,6-tetrahydropyrazolo[3,4-e]indazole-3-carboxamide.

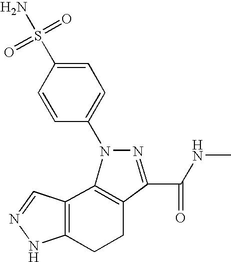

FABHRMS m/z 373.1087 (M+H, C₁₆H₁₇N₆O₃S requires 373.1083).

### EXAMPLE 232

This example illustrates the production of 6-phenyl-2,4,5,6-tetrahydropyrazolo[3,4-e]indazole-3-carboxamide.

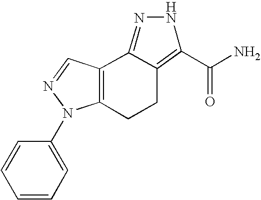

FABHRMS m/z 280.1166 (M+H, C₁₅H₁₄N₅O requires 280.1198).

### EXAMPLE 233

This example illustrates the production of 1-[4-(aminosulfonyl)phenyl]-8-[methyl(methylsulfonyl)amino]-4,5-dihydro-1H-benzo[g]indazole-3-carboxamide.

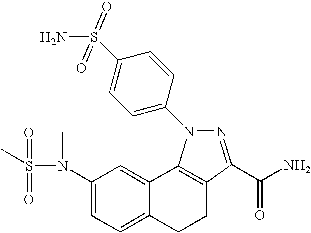

FABHRMS m/z 232.1061 (M+H, C₁₂H₁₄N₃O₂ requires 232.1086).

### EXAMPLE 234

This example illustrates the production of 1-[4-(aminosulfonyl)phenyl]-8-[methyl(methylsulfonyl)amino]-4,5-dihydro-1H-benzo[g]indazole-3-carboxamide.

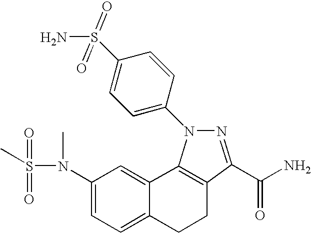

FABHRMS m/z 476.1063 (M+H, C₂₀H₂₂N₅O₅S₂ requires 476.1062).

### EXAMPLE 235

This example illustrates the production of 6-benzyl-1-phenyl-1,4,5,6-tetrahydropyrazolo[3,4-e]indazole-3-carboxamide.

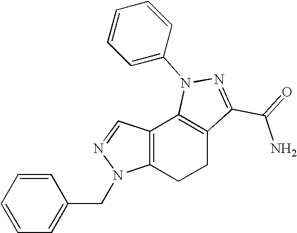

FABHRMS m/z 370.1680 (M+H, C₂₂H₂₀N₅O requires 370.1668).

### EXAMPLE 236

This example illustrates the production of 7-(acetylamino)-4,5-dihydro-2H-benzo[g]indazole-3-carboxylic acid.

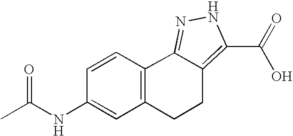

FABHRMS m/z 272.1043 (M+H, C₁₄H₁₄N₃O₃ requires 272.1035).

### EXAMPLE 237

This example illustrates the production of 1-phenyl-1,4,5,6-tetrahydropyrazolo[3,4-e]indazole-3-carboxylic acid.

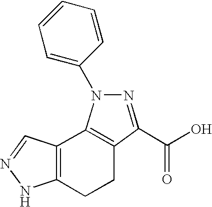

FABHRMS m/z 281.1044 (M+H, C₁₅H₁₃N₄O₂ requires 281.1039).

### EXAMPLE 238

This example illustrates the production of 6-(2-hydroxyethyl)-2,4,5,6-tetrahydropyrazolo[3,4-e]indazole-3-carboxamide.

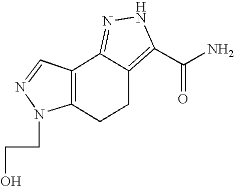

FABHRMS m/z 248.1172 (M+H, C₁₁H₁₄N₅O₂ requires 248.1147).

### EXAMPLE 239

This example illustrates the production of 7-amino-4,5-dihydro-2H-benzo[g]indazole-3-carboxylic acid.

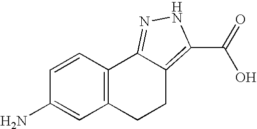

FABHRMS m/z 230.0940 (M+H, C₁₂H₁₂N₃O₂ requires 230.0930).

### EXAMPLE 240

This example illustrates the production of 1-[4-(methylsulfonyl)phenyl]-1,4,5,6-tetrahydropyrazolo[3,4-e]indazole-3-carboxylic acid.

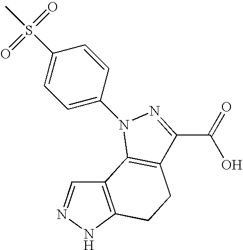

FABHRMS m/z 359.0833 (M+H, C₁₆H₁₅N₄O₄S requires 359.0814).

### EXAMPLE 241

This example illustrates the production of 6-[(benzyloxy)methyl]-2,4,5,6-tetrahydropyrazolo[3,4-e]indazole-3-carboxamide.

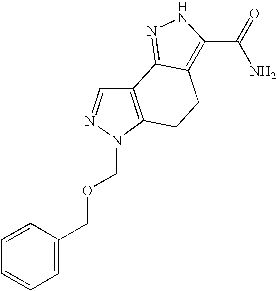

FABHRMS m/z 324.1460 (M+H, C₁₇H₁₈N₅O₂ requires 324.1461).

### EXAMPLE 242

This example illustrates the production of 7-[(benzyloxy)methyl]-2,4,5,7-tetrahydro-pyrazolo[3,4-e]indazole-3-carboxamide.

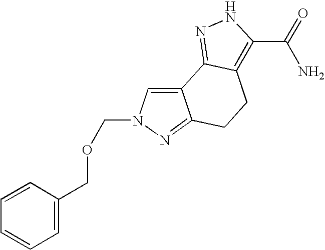

FABHRMS m/z 324.1460 (M+H, C₁₇H₁₈N₅O₂ requires 324.1461).

### EXAMPLE 243

This example illustrates the production of ethyl 6-(4-methylphenyl)-2,4,5,6-tetrahydropyrazolo[3,4-e]indazole-3-carboxylate.

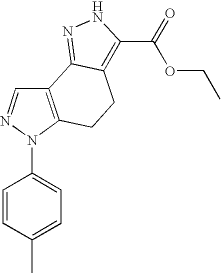

FABHRMS m/z 323.1539 (M+H, C₁₈H₁₉N₄O₂ requires 323.1508).

### EXAMPLE 244

This example illustrates the production of 6-(4-methylphenyl)-2,4,5,6-tetrahydropyrazolo[3,4-e]indazole-3-carboxylic acid.

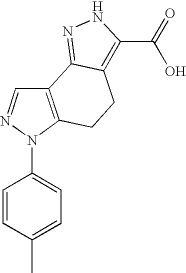

FABHRMS m/z 295.1193 (M+H, C₁₆H₁₅N₄O₂ requires 295.1195).

### EXAMPLE 245

This example illustrates the production of ethyl 7-allyl-2,4,5,7-tetrahydropyrazolo[3,4-e]indazole-3-carboxylate.

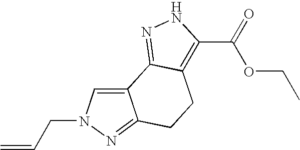

FABHRMS m/z 273.1340 (M+H, C₁₄H₁₇N₄O₂ requires 273.1346).

### EXAMPLE 246

This example illustrates the production of 7-allyl-2,4,5,7-tetrahydro-pyrazolo[3,4-e]indazole-3-carboxamide.

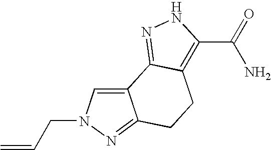

FABHRMS m/z 244.1206 (M+H, C₁₂H₁₄N₅O requires 244.1193).

### EXAMPLE 247

This example illustrates the production of ethyl 6-allyl-2,4,5,6-tetrahydropyrazolo[3,4-e]indazole-3-carboxylate.

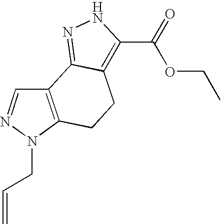

FABHRMS m/z 273.1338 (M+H, C₁₄H₁₇N₄O₂ requires 273.1346).

### EXAMPLE 248

This example illustrates the production of 6-allyl-2,4,5,6-tetrahydro-pyrazolo[3,4-e]indazole-3-carboxamide.

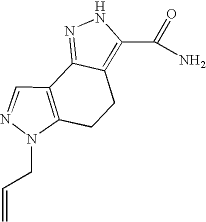

FABHRMS m/z 244.1226 (M+H, C₁₂H₁₄N₅O requires 244.1193).

### EXAMPLE 249

This example illustrates the production of 6-(ethoxymethyl)-2,4,5,6-tetrahydropyrazolo[3,4-e]indazole-3-carboxamide.

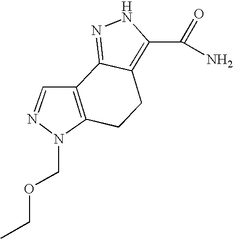

FABHRMS m/z 262.1282 (M+H, C₁₂H₁₆N₅O₂ requires 262.1299).

### EXAMPLE 250

This example illustrates the production of ethyl 7-isobutyl-2,4,5,7-tetrahydropyrazolo[3,4-e]indazole-3-carboxylate.

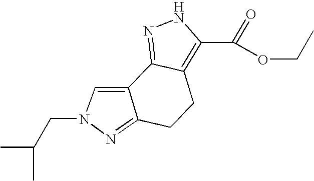

FABHRMS m/z 289.1624 (M+H, C₁₅H₂₁N₄O₂ requires 289.1659).

### EXAMPLE 251

This example illustrates the production of 7-isobutyl-2,4,5,7-tetrahydro-pyrazolo[3,4-e]indazole-3-carboxamide.

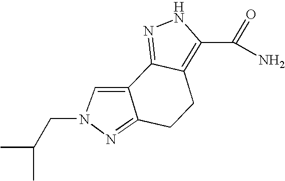

FABHRMS m/z 260.1505 (M+H, C₁₃H₁₈N₅O requires 260.1506).

### EXAMPLE 252

This example illustrates the production of 1-(2,2,2-trifluoroethyl)-1,4,5,6-tetrahydropyrazolo[3,4-e]indazole-3-carboxamide.

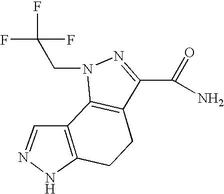

FABHRMS m/z 286.0902 (M+H, C₁₁H₁₁F₃N₅O requires 286.0910).

### EXAMPLE 253

This example illustrates the production of ethyl 1-(3-aminopropyl)-1,4,5,6-tetrahydropyrazolo[3,4-e]indazole-3-carboxylate.

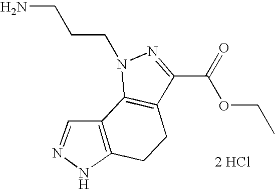

FABHRMS m/z 290.1585 (M+H, C₁₄H₂₀N₅O₂ requires 290.1612).

### EXAMPLE 254

This example illustrates the production of 1-(3-aminopropyl)-1,4,5,6-tetrahydropyrazolo[3,4-e]indazole-3-carboxylic acid.

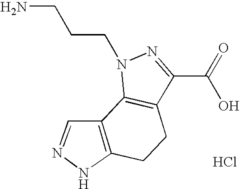

FABHRMS m/z 262.1278 (M+H, C₁₂H₁₆N₅O₂ requires 262.1299).

### EXAMPLE 255

This example illustrates the production of 1-(2-carboxyethyl)-1,4,5,6-tetrahydropyrazolo[3,4-e]indazole-3-carboxylic acid.

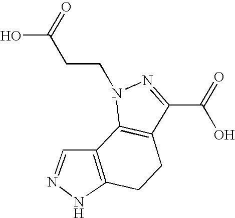

FABHRMS m/z 277.0931 (M+H, C₁₂H₁₃N₄O₄ requires 277.0931).

### EXAMPLE 256

This example illustrates the production of 1-(2-cyanoethyl)-1,4,5,6-tetrahydropyrazolo[3,4-e]indazole-3-carboxamide.

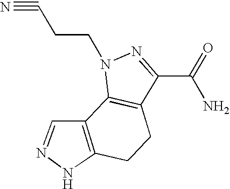

FABHRMS m/z 257.1174 (M+H, C₁₂H₁₃N₆O requires 257.1151).

### EXAMPLE 257

This example illustrates the production of ethyl 1-(2-cyanoethyl)-1,4,5,6-tetrahydropyrazolo[3,4-e]indazole-3-carboxylate hydrochloride.

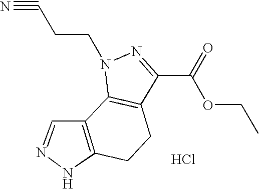

FABHRMS m/z 286.1304 (M+H, C₁₄H₁₆N₅O₂ requires 286.1345).

### EXAMPLE 258

This example illustrates the production of ethyl 1-(2-cyanoethyl)-7-trityl-1,4,5,6-tetrahydropyrazolo[3,4-e]indazole-3-carboxylate.

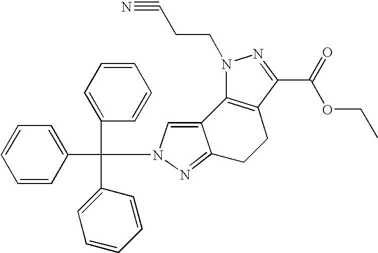

FABHRMS m/z 550.2219 (M+H, C₃₃H₃₀N₅O₂ requires 550.2202).

### EXAMPLE 259

This example illustrates the production of ethyl 1-(2-ethoxy-2-oxoethyl)-7-trityl-1,4,5,6-tetrahydropyrazolo[3,4-e]indazole-3-carboxylate.

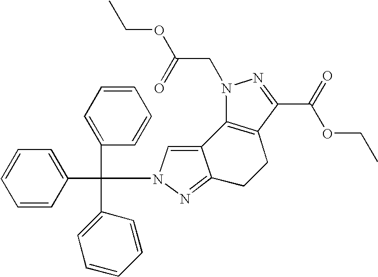

FABHRMS m/z 561.2454 (M+H, C₃₄H₃₃N₄O₄ requires 561.2502).

### EXAMPLE 260

This example illustrates the production of ethyl 2-(2-ethoxy-2-oxoethyl)-7-trityl-2,4,5,6-tetrahydropyrazolo[3,4-e]indazole-3-carboxylate.

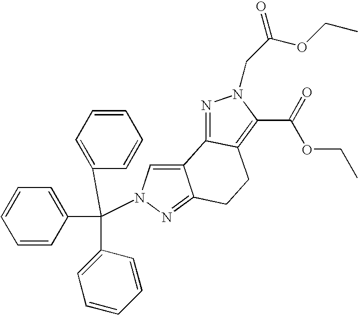

FABHRMS m/z 561.2495 (M+H, C₃₄H₃₃N₄O₄ requires 561.2496).

### EXAMPLE 261

This example illustrates the production of 2-(carboxymethyl)-2,4,5,6-tetrahydro-pyrazolo[3,4-e]indazole-3-carboxylic acid.

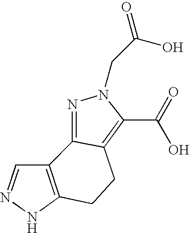

FABHRMS m/z 263.0787 (M+H, C₁₁H₁₁N₄O₄ requires 263.0775).

### EXAMPLE 262

This example illustrates the production of ethyl 2-(2-ethoxy-2-oxoethyl)-2,4,5,6-tetrahydropyrazolo[3,4-e]indazole-3-carboxylate hydrochloride.

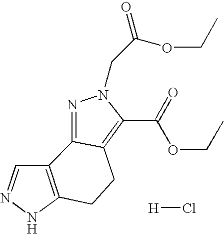

FABHRMS m/z 319.1422 (M+H, C₁₅H₁₉N₄O₄ requires 319.1401).

### EXAMPLE 263

This example illustrates the production of 3-(ethoxycarbonyl)-5,6-dihydropyrazolo[3,4-e]indazol-1 (4H)-yl]acetic acid.

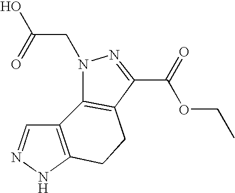

FABHRMS m/z 291.1051 (M+H, C₁₃H₁₅N₄O₄ requires 291.1088).

### EXAMPLE 264

This example illustrates the production of 1-(3-amino-3-oxopropyl)-1,4,5,6-tetrahydropyrazolo[3,4-e]indazole-3-carboxylic acid.

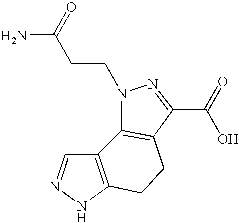

FABHRMS m/z 276.1107 (M+H, C₁₂H₁₄N₅O₃ requires 276.1091).

### EXAMPLE 265

This example illustrates the production of [3-(methoxycarbonyl)-5,6-dihydropyrazolo[3,4-e]indazol-2(4H)-yl]acetic acid.

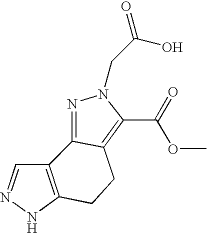

FABHRMS m/z 277.0901 (M+H, C₁₂H₁₃N₄O₄ requires 277.0931).

### EXAMPLE 266

This example illustrates the production of [3-(ethoxycarbonyl)-5,6-dihydro-pyrazolo[3,4-e]indazol-2(4H)-yl]acetic acid.

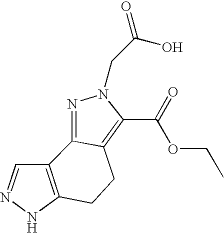

FABHRMS m/z 291.1066 (M+H, C₁₃H₁₅N₄O₄ requires 291.1088).

### EXAMPLE 267

This example illustrates the production of ethyl 2-(4-aminobutyl)-2,4,5,6-tetrahydro-pyrazolo[3,4-e]indazole-3-carboxylate.

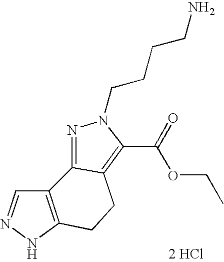

FABHRMS m/z 304.1785 (M+H, C₁₅H₂₂N₅O₂ requires 304.1768).

### EXAMPLE 268

This example illustrates the production of 1-(2-amino-2-oxoethyl)-1,4,5,6-tetrahydropyrazolo[3,4-e]indazole-3-carboxamide.

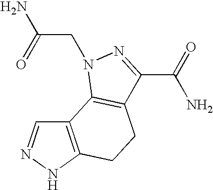

FABHRMS m/z 261.1101 (M+H, C₁₁H₁₃N₆O₂ requires 261.1095).

### EXAMPLE 269

This example illustrates the production of 1-(3-amino-3-oxopropyl)-1,4,5,6-tetrahydropyrazolo[3,4-e]indazole-3-carboxamide.

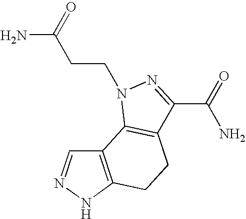

FABHRMS m/z 275.1268 (M+H, C₁₂H₁₅N₆O₂ requires 275.1251).

### EXAMPLE 270

This example illustrates the production of ethyl 8-(tert-butylamino)-2,4,5,6-tetrahydropyrazolo[3,4-e]indazole-3-carboxylate.

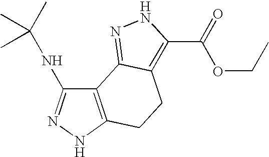

FABHRMS m/z 304.1739 (M+H, C₁₅H₂₂N₅O₂ requires 304.1768).

### EXAMPLE 271

This example illustrates the production of 2-(3-aminopropyl)-2,4,5,8-tetrahydropyrazolo[4,3-g]indazole-3-carboxylic acid hydrochloride.

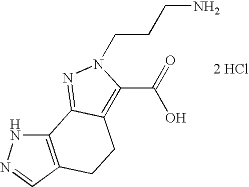

FABHRMS m/z 262.1304 (M+H, C₁₂H₁₆N₅O₂ requires 262.1299).

### EXAMPLE 272

This example illustrates the production of 1-(3-aminopropyl)-1,4,5,8-tetrahydro-pyrazolo[4,3-g]indazole-3-carboxylic acid hydrochloride.

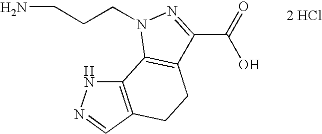

FABHRMS m/z 262.1288 (M+H, C₁₂H₁₆N₅O₂ requires 262.1299).

### EXAMPLE 273

This example illustrates the production of ethyl 7-trityl-2,4,5,7-tetrahydropyrazolo[4,3-g]indazole-3-carboxylate.

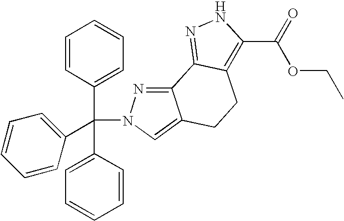

FABHRMS m/z 497.1899 (M+H, C₃₀H₂₇N₄O₂ requires 497.1948).

### EXAMPLE 274

This example illustrates the production of ethyl 1-[3-(tritylamino)propyl]-1,4,5,8-tetrahydropyrazolo[4,3-g]indazole-3-carboxylate.

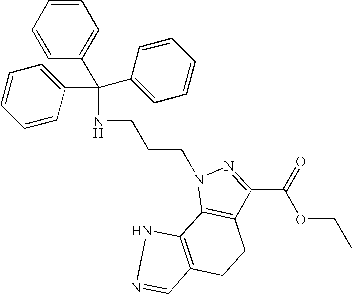

FABHRMS m/z 532.2707 (M+H, C₃₃H₃₄N₅O₂ requires 532.2672).

### EXAMPLE 275

This example illustrates the production of 1-(3-aminopropyl)-1,4,5,8-tetrahydropyrazolo[4,3-g]indazole-3-carboxamide.

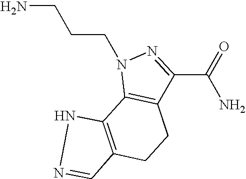

FABHRMS m/z 261.1449 (M+H, C₁₂H₁₇N₆O requires 261.1458).

### EXAMPLE 276 This example illustrates the production of ethyl 6-benzyl-1-[4-(methylsulfonyl)phenyl]-1,4,5,6-tetrahydropyrazolo[3,4-e]indazole-3-carboxylate.

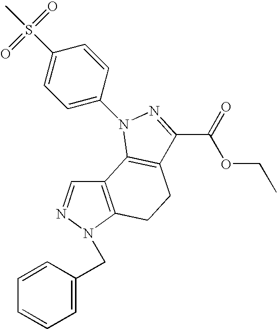

To N,N-dimethylacetamide dimethylacetal (250 mL) was added 1,3-cyclohexanedione (100 g, 0.89 mol). Contents were refluxed 2 hours, allowed to cool and concentrated in vacuo leaving an amber solid. After recrystallizing twice from EtOAc, the desired eneamine intermediate was obtained as light amber crystals, 64.2 g. The eneamine (21.7 g, 0.13 mol) and benzylhydrazine hydrochloride (25.5 g, 0.13 mol) were mixed in glacial acetic acid (200 mL) and stirred overnight. Contents were diluted-with water, extracted with EtOAc, dried over MgSO₄, and concentrated in vacuo leaving the desired tetralone intermediate as an amber oil, 37.2 g. The tetralone was reacted with diethyl oxalate according to the procedure for the production of ethyl (7-hydroxy-1-oxo-1,2,3,4-tetrahydronaphthalen-2-yl)(oxo)acetate, to give the desired diketoester intermediate as an amber oil. The diketoester was reacted with 4-methylsulfonylphenylhydrazine according to the procedure for the production of ethyl 8-hydroxy-4,5-dihydro-2H-benzo[g]indazole-3-carboxylate, to give the desired product as white crystals (53% yield). FABHRMS m/z 477.1587 (M+H, C₂₅H₂₅N₄O₄S requires 477.1597).

Anal. Calcd for C₂₄H₂₄N₄O₄S: C, 63.01; H, 5.08; N, 11.76. Found: C, 62.93; H, 4.92; N, 11.77.

### EXAMPLE 277

This example illustrates the production of ethyl 1-[4-(methylsulfonyl)-phenyl]-1,4,5,6-tetrahydropyrazolo[3,4-e]indazole-3-carboxylate.

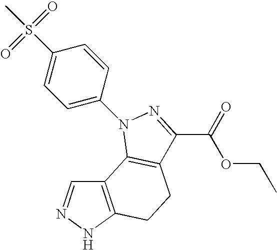

Ethyl 6-benzyl-1-[4-(methylsulfonyl)phenyl]-1,4,5,6-tetrahydropyrazolo[3,4-e]indazole-3-carboxylate (2.5 g, 0.005 mol), Pearleman's catalyst (1.0 g) and glacial acetic acid (30 mL) were shaken at 55 psi H₂ and 55° C., overnight. Contents were allowed to cool and filtered through clay. The filtrate was concentrated in vacuo and the residue was triturated with methanol and filtered to give the desired product as a white solid, 609 mg (32% yield). FABHRMS m/z 387.1161 (M+H, C₁₈H₁₉N₄O₄S requires 387.1127).

Anal. Calcd for C₁₈H₁₈N₄O₄S (0.8H₂O): C, 53.94; H, 4.93; N, 13.98. Found: C, 53.97; H, 4.71; N, 13.71.

### EXAMPLE 278

This example illustrates the production of 1-[4-(methylsulfonyl)phenyl]-1,4,5,6-tetrahydropyrazolo[3,4-e]indazole-3-carboxamide.

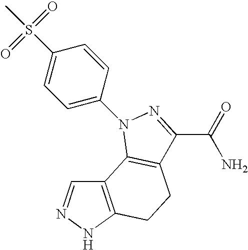

Ethyl 1-[4-(methylsulfonyl)phenyl]-1,4,5,6-tetrahydropyrazolo[3,4-e]indazole-3-carboxylate was reacted according to the procedure for the production of 8-hydroxy-4,5-dihydro-2H-benzo[g]indazole-3-carboxamide to give the desired product as a white solid (58% yield).

Anal. Calcd for C₁₆H₁₅N₅O₃S (0.5H₂O): C, 52.45; H, 4.40; N, 19.11. Found: C, 52.42; H, 4.41; N, 18.63.

### EXAMPLE 279

This example illustrates the production of 1,5,6,7-tetrahydro-4H-indazol-4-one.

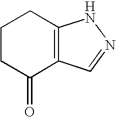

To N,N-dimethylacetamide dimethylacetal (250 mL) was added 1,3-cyclohexanedione (100 g, 0.89 mol). Contents were refluxed 2 hours, allowed to cool and concentrated in vacuo leaving amber solid. After recrystallizing twice from EtOAc, the desired eneamine intermediate was obtained as light amber crystals, 64.2 g. The eneamine (5.0 g, 0.03 mol) and p-tosylhydrazine hydro-chloride (5.6 g, 0.3 mol) were stirred in methanol overnight. 4N HCl in dioxane (100 mL) was added and contents were stirred overnight. A white solid was filtered to give the desired product, 4.5 g (87% yield). FABHRMS m/z 137.0738 (M+H, C₇H₉N₂O requires 137.0715). ¹H NMR (DMSO-d₆/300 MHz): 7.90 (s, 1H); 2.90-2.80 (m, 2H); 2.40-2.30 (m, 2H); 2.10-2.00 (m, 3H).

Anal. Calcd for C₇H₈N₂O (0.05H₂O): C, 61.35; H, 5.96; N, 20.44. Found: C, 61.25; H, 5.98; N, 20.53.

### EXAMPLE 280

This example illustrates the production of 2-trityl-2,5,6,7-tetrahydro-4H-indazol-4-one.

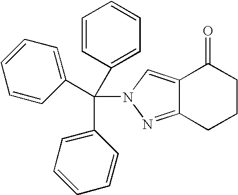

To 7-oxo-4,5,6,7-tetrahydro-1H-indazole (2.0 g, 0.012 mol) and triethylamine (4.4 mL, 0.031 mol) in CH₂Cl₂ (25 mL) was added dropwise trityl chloride (4.3 g, 0.0155 mol) in CH₂Cl₂ (20 mL). Contents were stirred overnight, washed with water and the CH₂Cl₂ layer was concentrated in vacuo. The residue was partitioned between ether and water. The ether layer was washed with brine, dried over MgSO₄ and concentrated in vacuo leaving an amber oil. Trituration with hexanes gave the desired product as a white solid, 3.6 g (79% yield). FABHRMS m/z 401.1608 (M+H, C₂₆H₂₃N₂O requires 401.1630). ¹H NMR (CDCl₃/300 MHz): 7.90 (s, 1H); 7.40-7.10 (m, 15H); 2.90-2.80 (m, 2H); 2.55-2.45 (m, 2H); 2.20-2.10 (m, 2H).

Anal. Calcd for C₂₆H₂₂N₂O: C, 82.51; H, 5.86; N, 7.4. Found: C, 82.36; H, 6.10; N, 7.32.

### EXAMPLE 281

This example illustrates the production of 6-methyl-2,4,5,6-tetrahydro-pyrazolo[3,4-e]indazole-3-carboxamide.

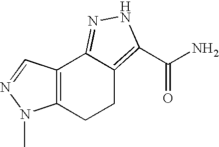

FABHRMS m/z 218.1051 (M+H, C₁₀H₁₂N₅O requires 218.1042).

### EXAMPLE 282

This example illustrates the production of ethyl 2-trityl-4-oxo-4,5,6,7-tetrahydro-2H-indazol-5-yl](oxo)acetate.

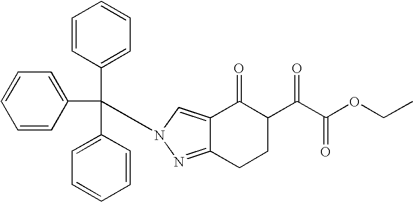

1-trityl-1,5,6,7-tetrahydro-4H-indazol-4-one- or 1-trityl-1,5,6,7-tetrahydro-4H-indazol-4-one was reacted according to the procedure for the production of ethyl (7-hydroxy-1-oxo-1,2,3,4-tetrahydronaphthalen-2-yl)(oxo)acetate to give the desired product as yellow crystals (82% yield). FABHRMS m/z 479.1971 (M+H, C₃₀H₂₇N₂O₄ requires 479.1965). ¹H NMR (CDCl₃/300 MHz): 7.97 (s, 1H); 7.40-7.10 (m, 15H); 4.40 (q, 2H); 3.15-3.05 (m, 2H); 2.95-2.85 (m, 2H); 1.45 (t, 3H).

Anal. Calcd for C₃₀H₂₆N₂O₄: C, 75.30; H, 5.48; N, 5.85. Found: C, 75.11; H, 5.38; N, 5.73.

### EXAMPLE 283

This example illustrates the production of ethyl 2,4,5,6-tetrahydro-pyrazolo[3,4-e]indazole-3-carboxylate.

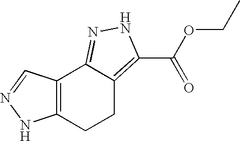

Ethyl oxo(4-oxo-1-trityl-4,5,6,7-tetrahydro-1H-indazol-5-yl)acetate- or ethyl oxo(4-oxo-2-trityl-4,5,6,7-tetrahydro-2H-indazol-5-yl)acetate or ethyl oxo(4-oxo-2-trityl-4,5,6,7-tetrahydro-2H-indazol-5-yl)acetate was reacted according to the procedure for the production of ethyl 8-hydroxy-4,5-dihydro-2H-benzo[g]indazole-3-carboxylate, at 70° C. to give the desired product (67% yield). ¹H NMR (DMSO-d₆/300 MHz): 7.78 (s, 1H); 4.30 (q, 2H); 3.05-2.95 (m, 2H); 2.90-2.80 (m, 2H); 1.35 (t, 3H).

Anal. Calcd for C₁₁H₁₃N₄O₂ (2HOAc): C, 51.13; H, 5.72; N, 15.90. Found: C, 51.09; H, 5.80; N, 16.09.

### EXAMPLE 284

This example illustrates the production of 2,4,5,6-tetrahydropyrazolo[3,4-e]indazole-3-carboxamide.

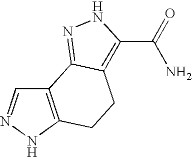

Ethyl 2,4,5,6-tetrahydropyrazolo[3,4-e]indazole-3-carboxylate was reacted according to the procedure for the production of 8-hydroxy-4,5-dihydro-2H-benzo[g]indazole-3-carboxamide to give the desired as an off-white solid (97% yield). FABHRMS m/z 204.0877 (M+H, C₉H₁₀N₅O requires 204.0885).

¹H NMR (DMSO-d₆+TFA/300 MHz): 7.85 (s, 1H); 7.30 (brs, 2H); 3.05-2.95 (m, 2H); 2.90-2.80 (m, 2H).

Anal. Calcd for C₉H₉N₅O: C, 53.20; H, 4.46; N, 34.47. Found: C, 53.14; H, 4.47; N, 34.57.

### EXAMPLE 285

This example illustrates the production of ethyl [1-(2-hydroxyethyl)-4-oxo-4,5,6,7-tetrahydro-1H-indazol-5-yl](oxo)acetate.

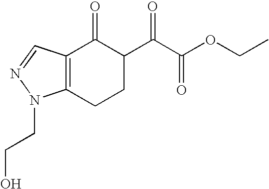

To N,N-dimethylacetamide dimethylacetal (250 mL) was added 1,3-cyclohexanedione (100 g, 0.89 mol). Contents were refluxed 2 hours, allowed to cool and concentrated in vacuo leaving amber solid. After recrystallizing twice from EtOAc, the desired eneamine intermediate was obtained as light amber crystals, 64.2 g. The eneamine (20.0 g, 0.12 mol) and hydroxyethylhydrazine (7.2 g, 0.12 mol) were mixed in methanol (50 mL) and stirred 72 hours. Contents were concentrated in vacuo leaving an oil, 13.4 g which was purified by silica gel chromatography eluting with 5% MeOH/EtOAc to give the desired tetralone intermediate as a yellow solid, 17.6 g (81% yield). The tetralone was reacted according to the procedure for the production of ethyl (7-hydroxy-1-oxo-1,2,3,4-tetrahydronaphthalen-2-yl)(oxo)acetate to give the desired product as a white solid (38% yield). FABHRMS m/z 281.1148 (M+H, C₁₃H₁₇N₂O₅ requires 281.1137).

Anal. Calcd for C₁₃H₁₆N₂O₅: C, 55.71; H, 5.75; N, 9.99. Found: C, 55.63; H, 5.67; N, 9.96.

### EXAMPLE 286

This example illustrates the production of ethyl 6-(2-hydroxyethyl)-2,4,5,6-tetrahydropyrazolo[3,4-e]indazole-3-carboxylate acetate.

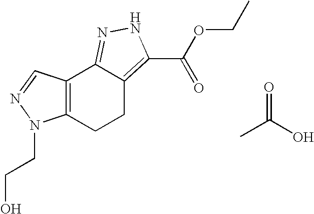

Ethyl 6-(2-hydroxyethyl)-2,4,5,6-tetrahydropyrazolo[3,4-e]indazole-3-carboxylate acetate was prepared according to the procedure for the production of ethyl 8-hydroxy-4,5-dihydro-2H-benzo[g]indazole-3-carboxylate, to give the desired product as a white solid (80% yield). FABHRMS m/z 277.1296 (M+H, C₁₃H₁₇N₄O₃ requires 277.1301).

Anal. Calcd for C₁₃H₁₆N₄O₃ (HOAc):C, 53.56; H, 5.99; N, 16.66. Found: C, 53.45; H, 5.71; N, 16.60.

### EXAMPLE 287

This example illustrates the production of 6-(2-hydroxyethyl)-2,4,5,6-tetrahydro-pyrazolo[3,4-e]indazole-3-carboxylic acid.

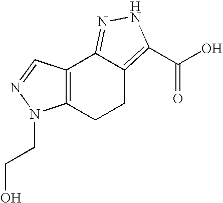

6-(2-hydroxyethyl)-2,4,5,6-tetrahydropyrazolo[3,4-e]indazole-3-carboxylic acid was prepared according to the procedure for the production of 7-hydroxy-4,5-dihydro-2H-benzo[g]indazole-3-carboxylic acid to give the desired product as a white solid (83% yield). FABHRMS m/z 249.0988 (M+H, C₁₁H₁₃N₄O₃ requires 249.0988).

Anal. Calcd for C₁₁H₁₂N₄O₃: C, 53.22; H, 4.87; N, 22.57. Found: C, 52.86; H, 4.83; N, 22.49.

### EXAMPLE 288

This example illustrates the production of ethyl 1-[4-(aminosulfonyl)-phenyl]-6-benzyl-1,4,5,6-tetrahydropyrazolo[3,4-e]indazole-3-carboxylate.

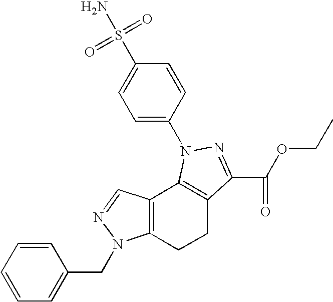

Ethyl 1-[4-(aminosulfonyl)phenyl]-6-benzyl-1,4,5,6-tetrahydropyrazolo[3,4-e]indazole-3-carboxylate- or ethyl 1-[4-(aminosulfonyl)phenyl]-6-benzyl-1,4,5,6-tetrahydropyrazolo[3,4-e]indazole-3-carboxylate was prepared according to the procedures for the production of ethyl (7-hydroxy-1-oxo-1,2,3,4-tetrahydronaphthalen-2-yl)(oxo)acetate and ethyl 1-[4-(aminosulfonyl)phenyl]-8-hydroxy-4,5-dihydro-1H-benzo[g]indazole-3-carboxylate to give the desired as a light tan solid (61% yield). FABHRMS m/z 478.1549 (M+H, C₂₄H₂₄N₅O₄S requires 478.1551). ¹H NMR (DMSO-d₆+5%TFA/300 MHz): 7.98 (AB quartet, 4H); 7.38-7.20 (m, 3H); 7.20-7.10 (m, 2H); 5.38 (s, 2H); 4.25 (q, 2H); 3.20-2.90 (m, 4H); 1.25 (t, 3H).

Anal. Calcd for C₂₄H₂₃N₅O₄S: C, 60.36; H, 4.85; N, 14.67. Found: C, 60.60; H, 4.86; N, 14.71.

### EXAMPLE 289

This example illustrates the production of ethyl 1-[4-(aminosulfonyl)-phenyl]-1,4,5,6-tetrahydropyrazolo[3,4-e]indazole-3-carboxylate.

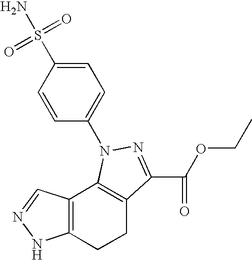

Ethyl 1-[4-(aminosulfonyl)phenyl]-6-benzyl-1,4,5,6-tetrahydropyrazolo[3,4-e]indazole-3-carboxylate- or ethyl 1-[4-(aminosulfonyl)phenyl]-6-benzyl-1,4,5,6-tetrahydropyrazolo[3,4-e]indazole-3-carboxylate (4.2 g, 0.009 mol), Pearleman's catalyst (1.5 g), glacial acetic acid (20 mL) and DMF (40 mL) were shaken at 55 psi H₂ for 72 hours. Contents were filtered through clay and the filtrate was concentrated in vacuo leaving a grey solid. The grey solid was triturated with acetonitrile to give an off-white solid, 3.0 g (86% yield). FABHRMS m/z 388.1080 (M+H, C₁₇H₁₈N₅O₄S requires 388.1124). ¹H NMR (DMSO-d₆/300 MHz): 7.96 (AB quartet, 4H); 7.55 (br s, 2H); 7.35 (br s, 1H); 4.35 (q, 2H); 3.30 (s, 2H); 3.15-3.05 (m, 2H); 3.00-2.85 (m, 2H); 1.35 (t, 3H).

Anal. Calcd for C₁₇H₁₇N₅O₄S: C, 52.70; H, 4.42; N, 18.08. Found: C, 52.47; H, 4.18; N, 17.89.

### EXAMPLE 290

This example illustrates the production of 1-[4-(aminosulfonyl)phenyl]-1,4,5,6-tetrahydropyrazolo[3,4-e]indazole-3-carboxamide.

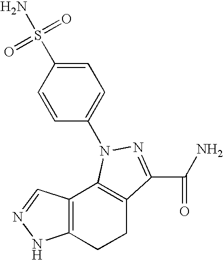

Ethyl 1-[4-(aminosulfonyl)phenyl]-1,4,5,6-tetrahydropyrazolo[3,4-e]indazole-3-carboxylate- or ethyl 1-[4-(aminosulfonyl)phenyl]-1,4,5,6-tetrahydropyrazolo[3,4-e]indazole-3-carboxylate was reacted according to the procedure for the production of 8-hydroxy-4,5-dihydro-2H-benzo[g]indazole-3-carboxamide to give the desired product as a white solid (86% yield). FABHRMS m/z 359.0939 (M+H, C₁₅H₁₅N₆O₃S requires 359.0926). ¹H NMR (DMSO-d₆/300 MHz): 7.99 (AB quartet, 4H); 7.58 (s, 1H); 7.50 (s, 1H); 7.35 (s, 1H); 3.35 (s, 2H); 3.10-3.00 (m, 2H); 2.90-2.80 (m, 2H).

Anal. Calcd for C₁₅H₁₄N₆O₃S: C, 50.27; H, 3.94; N, 23.45. Found: C, 50.07; H, 3.73; N, 23.08.

### EXAMPLE 291

This example illustrates the production of 1-[4-(aminosulfonyl)phenyl]-1,4,5,6-tetrahydropyrazolo[3,4-e]indazole-3-carboxylic acid.

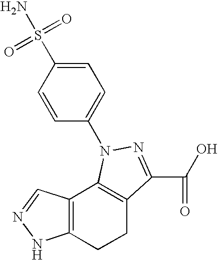

Ethyl 1-[4-(aminosulfonyl)phenyl]-1,4,5,6-tetrahydropyrazolo[3,4-e]indazole-3-carboxylate- or ethyl 1-[4-(aminosulfonyl)phenyl]-1,4,5,6-tetrahydropyrazolo[3,4-e]indazole-3-carboxylate was reacted according to the procedure of 7-hydroxy-4,5-dihydro-2H-benzo[g]indazole-3-carboxylic acid to give the desired product as a white solid (85% yield). FABHRMS m/z 360.0762 (M+H, C₁₅H₁₄N₅O₄S requires 360.0767).

Anal. Calcd for C₁₅H₁₃N₅O₄S: C, 46.03; H, 3.35; N, 17.89. Found: C, 46.67; H, 3.64; N, 17.93.

### EXAMPLE 292

This example illustrates the production of ethyl 6-benzyl-1-phenyl-1,4,5,6-tetrahydropyrazolo[3,4-e]indazole-3-carboxylate.

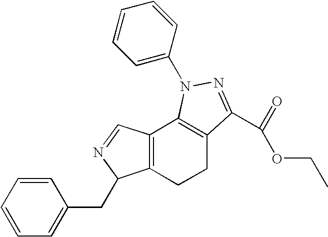

To N,N-dimethylacetamide dimethylacetal (250 mL) was added 1,3-cyclohexanedione (100 g, 0.89 mol). Contents were refluxed 2 hours, allowed to cool and concentrated in vacuo leaving an amber solid. After recrystallizing twice from EtOAc, the desired eneamine intermediate was obtained as light amber crystals, 64.2 g. The eneamine (21.7 g, 0.13 mol) and benzylhydrazine hydrochloride (25.5 g, 0.13 mol) were mixed in glacial acetic acid (200 mL) and stirred overnight. Contents were diluted with water, extracted with EtOAc, dried over MgSO₄, and concentrated in vacuo leaving the desired tetralone intermediate as an amber oil, 37.2 g. The tetralone was reacted with diethyl oxalate according to the procedure for the production of ethyl (7-hydroxy-1-oxo-1,2,3,4-tetrahydronaphthalen-2-yl)(oxo)acetate, to give the desired diketoester intermediate as an amber oil. The diketoester was reacted with phenylhydrazine according to the procedure for the production of ethyl 8-hydroxy-4,5-dihydro-2H-benzo[g]indazole-3-carboxylate, to give the desired product as white crystals (14% yield). FABHRMS m/z 399.1826 (M+H, C₂₄H₂₃N₄O₂ requires 399.1821).

Anal. Calcd for C₂₄H₂₂N₄O₂: C, 72.34; H, 5.57; N, 14.06. Found: C, 72.14; H, 5.41; N, 13.98.

### EXAMPLE 293

This example illustrates the production of ethyl 1-phenyl-1,4,5,6-tetrahydro-pyrazolo[3,4-e]indazole-3-carboxylate.

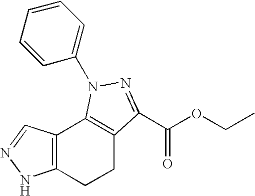

Ethyl 6-benzyl-1-phenyl-1,4,5,6-tetrahydropyrazolo[3,4-e]indazole-3-carboxylate (3.4 g, 0.009 mol) and Pearleman's catalyst (1.0 g) in ethanol (100 mL) were shaken at 55 psi H₂ and 55° C. for 72 hours. Contents were allowed to cool and filtered through clay. The filtrate was concentrated in vacuo leaving a white solid. Triteration of the solid with methanol gave the desired as a white solid, 1.0 g (36% yield). FABHRMS m/z 309.1343 (M+H, C₁₇H₁₇N₄O₂ requires 309.1352).

Anal. Calcd for C₁₇H₁₆N₄O₂: C, 66.22; H, 5.23; N, 18.17. Found: C, 66.06; H, 5.41; N, 17.58.

### EXAMPLE 294

This example illustrates the production of 1-phenyl-1,4,5,6-tetrahydro-pyrazolo[3,4-e]indazole-3-carboxamide.

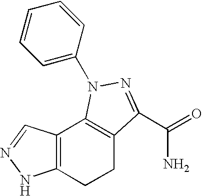

1-phenyl-1,4,5,6-tetrahydropyrazolo[3,4-e]indazole-3-carboxamide was prepared according to the procedure for the production of 8-hydroxy-4,5-dihydro-2H-benzo[g]indazole-3-carboxamide, at 100° C., to give the desired product as a grey solid (72% yield). FABHRMS m/z 280.1208 (M+H, C₁₅H₁₄N₅O requires 280.1198).

Anal. Calcd for C₁₅H₁₃N₅O (0.5H₂O): C, 62.49; H, 4.89; N, 24.29. Found: C, 62.94; H, 4.83; N, 23.76.

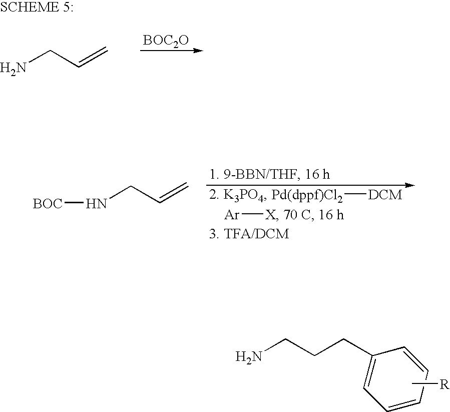

### EXAMPLE 295

This example illustrates the production of Boc-allyl amine. Boc-anhydride (1M THF, 70 mmol, 70 mL) was added to allyl amine (70 mmol, 4.0 g, 5.26 mL) slowly with stirring. The reaction solution stirred at 23° C. for 16 hours. The THF was removed in vacuo and the residue was reconstituted in dichloromethane (20 mL) and washed with water and brine (1×25 mL). The organic layer was dried (MgSO₄) and concentrated in vacuo. The product was isolated as a white low melting solid (10.25 g, 99% yield): ¹HNMR (409 MHz, CDCl₃) δ 5.853-5.770 (m, 1H), 5.278-5.069 (qm, 2H), 3.723 (bs, 2H), 1.429 (s, 9H).

### EXAMPLE 296

This example illustrates the production of 3-(4-tert-butylphenyl)-propylamine.

Tert-butyl allylcarbamate (7.0 mmol, 1.10 g) was weighed into a 100 mL flask with-stir bar and 9-BBN solution (0.5M, THF, 7.7 mmol, 15.4 mL) was syringed. Carefully into the neat amine. The reaction stirred at 23° C. for 16 hours. The potassium phosphate (2M, 10.5 mmol, 5.25 mL) was added to the reaction mixture and stirred at 23° C. for 10 minutes. [1,1′-Bis(diphenylphosphino)ferrocene]dichloropalladium(II)/DCM complex (0.21 mmol, 171 mg) and the 4-t-butyl-phenylbromide (7.0 mmol, 1.49 g, 1.21 mL) were added to the basic reaction mixture and the slurry was heated to relfux for 16 hours. The crude reaction mixture was purified using a 20 g flash silica plug (eluting with 100% hexane to 50% ethylacetate/hexane). The isolated pale brown oil (2.96 g) was dissolved in 25%TFA/DCM (15 mL) and stirred for 30 minutes at 23° C. The reaction mixture was concentrated in vacuo, reconstituted in dichloromethane (20 mL) and washed with saturated bicarbonate solution (3×30 mL). The organic layer was concentrated in vacuo. The crude product was purified using a 20 g flash silica plug (eluting with 100% hexane to 100% ethyl acetate to 100% methanol). Isolated the product as a light tan solid (1.25 g, 94% yield): ¹HNMR (400 MHz, DMSO) δ 7.911 (bs, 2H), 7.200 (q, (a,b), 4H), 2.762 (m, 2H), 2.579 (t, 2H), 1.811 (m, 2H), 1.240 (s, 9H): m/z 192 (M+H).

### EXAMPLE 297

This example illustrates the production of 3-(4-chlorophenyl)propylamine.

The product was isolated as a tan semi-solid (231 mg, 45% yield): ¹HNMR (400 MHz, DMSO) δ 7.300 (q, a,b, 4H), 2.631 (m, 4H), 1.724 (m, 2H): m/z 170 (M+H).

### EXAMPLE 298

This example illustrates the production of 3-(4-bromophenyl)propylamine.

The product was isolated as a tan semi-solid (310 mg, 48% yield): ¹HNMR (400 MHz, DMSO) δ 7.380 (q, a,b, 4H), 2.639 (q, 4H), 1.724 (m, 2H): m/z 216(M+H).

### EXAMPLE 299

This example illustrates the production of 3-(3-chlorophenyl)propylamine.

The product was isolated as a tan semi-solid (244 mg, 48% yield): ¹HNMR (400 MHz, DMSO) δ 7.323 (m, 3H), 7.220 (d, 1H), 2.664 (m, 4H), 1.796 (t, 2H): m/z 170 (M+H).

### EXAMPLE 300

This example illustrates the production of 3-[4-(methylsulfonyl) phenyl]propylamine. The product was isolated as a tan semi-solid (188 mg, 29% yield): ¹HNMR (400 MHz, DMSO) δ 7.610 (q, a,b, 4H), 3.220 (s, 3H), 2.783 (m, 4H), 1.873 (m, 2H): m/z 214 (M+H).

### EXAMPLE 301

This example illustrates the production of 3-(1,1′-biphenyl-4-yl)propylamine.

The product was isolated as a tan semi-solid (485 mg, 76% yield): ¹HNMR (400 MHz, DMSO) δ 7.649(m, 4H), 7.483 (t, 2H), 7.374 (m, 3H), 2.842 (t, 2H), 2.686 (t, 2H), 1.894 (t, 2H): m/z 212 (M+H).

The following compounds were synthesized in the same manner as 2-(3-{[2-(2-chlorophenyl)ethyl]amino}propyl)-4,5-dihydro-2H-pyrazolo[3,4-f]isoquinoline-3-carboxylic acid trifluoroacetate (cyclic pyrazole Bromide alkylation of amines) using the previously described amines.

### EXAMPLE 302

This example illustrates the production-of 2-(3-{[3-(4-tert-butylphenyl)propyl]amino}propyl)-4,5-dihydro-2H-pyrazolo[3,4-f]isoquinoline-3-carboxylic acid trifluoroacetate.

The product was isolated as a white amorphous solid (148 mg, 30% yield):

¹HNMR (400 MHz, DMSO) δ 8.685 (s, 1H), 8.608 (d, 1H), 7.840 (d, 1H), 7.284 (d, 2H), 7.094 (d, 2H), 4.627 (t, 2H), 3.010 (s, 4H), 2.931 (m, 2H), 2.860 (m 2H0, 2.551 (t, 2H), 2.126 (m, 2H), 1.807 (m, 2H), 1.222 (s, 9H): m/z 447 (M+H).

### EXAMPLE 303

This example illustrates the production of 2-(3-{[3-(4-chlorophenyl)propyl]amino}propyl)-4,5-dihydro-2H-pyrazolo[3,4-f]isoquinoline-3-carboxylic acid trifluoroacetate.

The product was isolated as a white amorphous solid (84 mg, 18% yield):

¹HNMR (400 MHz, DMSO) δ 8.726 (bs, 1H), 8.644 (d, 1H), 7.895 (d, 1H), 7.334 (d, 2H), 7.211 (d, 2H), 4.632 (t, 2H), 3.025 (s, 4H), 2.931 (m, 2H), 2.860 (m, 2H), 2.593 (t, 2H), 2.126 (m, 2H), 1.807 (m, 2H): m/z 425 (M+H).

### EXAMPLE 304

This example illustrates the production of 2-(3-{[3-(4-bromophenyl)propyl]amino}propyl)-4,5-dihydro-2H-pyrazolo[3,4-f]isoquinoline-3-carboxylic acid trifluoroacetate.

The product was isolated as a white amorphous solid (71 mg, 14% yield):

¹HNMR (400 MHz, DMSO) δ 8.726 (bs, 1H), 8.644 (d, 1H), 7.895 (d, 1H), 7.449 (d, 2H), 7.154 (d, 2H), 4.632 (t, 2H), 3.006 (s, 4H), 2.931 (m, 2H), 2.860 (m, 2H), 2.593 (t, 2H), 2.126 (m, 2H), 1.807 (m, 2H): m/z 471 (M+H).

### EXAMPLE 305

This example illustrates the production of 2-(3-{[3-(3-chlorophenyl)propyl]-amino}propyl)-4,5-dihydro-2H-pyrazolo[3,4-f]isoquinoline-3-carboxylic acid trifluoroacetate.

The product was isolated as a white amorphous solid (58 mg, 12% yield):

¹HNMR (400 MHz, DMSO) δ 8.727 (s, 1H), 8.644 (d, 1H), 7.899 (d, 1H), 7.268 (m, 3H), 7.156 (d, 1H), 4.634 (t, 2H), 3.027 (s, 4H), 2.931 (m, 2H), 2.862 (m, 2H), 2.613 (t, 2H), 2.153 (m, 2H), 1.830 (m, 2H): m/z 425 (M+H).

### EXAMPLE 306

This example illustrates the production of 2-[3-({3-[4-(methylsulfonyl)phenyl]propyl}amino)propyl]-4,5-dihydro-2H-pyrazolo[3,4-f]isoquinoline-3-carboxylic acid trifluoroacetate.

The product was isolated as a white amorphous solid (52 mg, 12% yield):

¹HNMR (400 MHz, DMSO) δ 8.662 (bs, 1H), 8.591 (d, 1H), 7.841 (d, 2H), 7.806 (d, 1H), 7.466 (d, 2H), 4.626 (t, 2H), 3.460 (dq, 4H), 3.159 (s, 3H), 2.931 (m, 2H), 2.860 (m, 2H), 2.718 (t, 2H), 2.126 (m, 2H), 1.857 (m, 2H): m/z 469 (M+H).

### EXAMPLE 307

This example illustrates the production of 2-(3-{[3-(1,1′-biphenyl-4-yl)propyl]amino}propyl)-4,5-dihydro-2H-pyrazolo[3,4-f]isoquinoline-3-carboxylic acid trifluoroacetate.

The product was isolated as a white amorphous solid (52 mg, 12% yield):

¹HNMR (400 MHz, DMSO) δ 8.684 (s, 1H), 8.600 (d, 1H), 7.839 (d, 1H), 7.613-7.555 (m, 4H), 7.422 (t, 2H), 7.320 (t, 1H), 7.277 (d, 2H), 4.634 (t, 2H), 3.008 (s, 4H), 2.940 (m, 4H), 2.645 (t, 2H), 2.140 (m, 2H), 1.865 (m, 2H): m/z 467 (M+H).

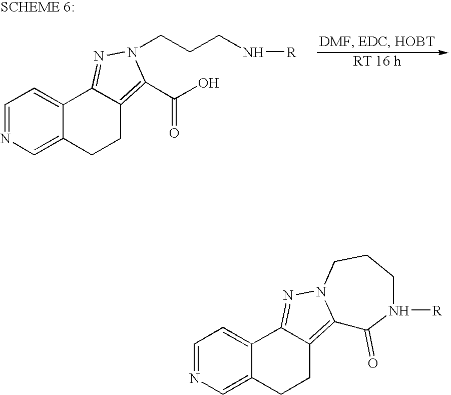

### EXAMPLE 308

This example illustrates the production of 8-[2-(2-chlorophenyl)ethyl]-5,6,8,9,10,11-hexahydro-7H-[1,4]diazepino[1′,2′:1,5]pyrazolo[3,4-f]isoquinolin-7-one trifluoroacetate.

2-(3-{[2-(2-chlorophenyl)ethyl]amino}propyl)-4,5-dihydro-2H-pyrazolo[3,4-f]isoquinoline-3-carboxylic acid trifluoroacetate (0.38 mmol, 200 mg) was dissolved in DMF (5.0 mL) and EDC-HCl (0.57 mmol, 109 mg), and HOBT (0.46 mmol, 62 mg) were added. The reaction mixture stirred at 23° C. for 16 hours. The reaction mixture was diluted with ethyl acetate (20 mL) and washed with water (2×25 mL). The organic layer was concentrated in vacuo, reconstituted in acetonitrile and purified using reverse phase chromatography. The product was isolated as a white amorphous solid (36.0 mg, 24% yield).

¹HNMR (400 MHz, DMSO) δ 8.805 (s, 1H), 8.726 (d, 1H), 8.045 (d, 1H), 7.434 (t, 2H), 7.319 (m, 2H), 4.406 (t, 2H), 3.789 (t, 2H), 3.378 (t, 2H), 3.089 (t, 4H), 2.917 (t, 2H), 2.149 (t, 2H): m/z 394 (M+H).

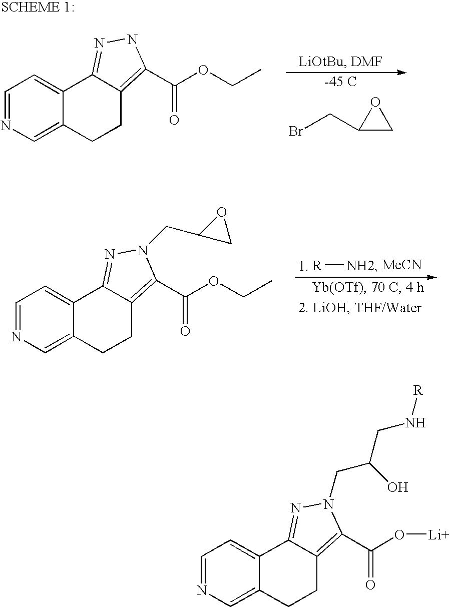

### EXAMPLE 309

This example illustrates the production of ethyl 2-(oxiran-2-ylmethyl)-4,5-dihydro-2H-pyrazolo[3,4-f]isoquinoline-3-carboxylate.

The pyrazole ester (1.23 mmol, 300 mg) was dissolved in DMF (10 mL) and cooled to −45° C. in a dry ice/acetonitrile bath. Lithium tert-butoxide (1M THF, 1.85 mmol, 1.85 mL) was added slowly being sure to maintain −45° C. The cold solution stirred for 1.5 h. Epibromohydrin (1.85 mmol, 0.253 g, 159 μL) was added via syringe to the cold DMF solution and the ice bath was removed. The reaction was allowed to warm to 23° C. over 16 h. The crude reaction was diluted with ethyl acetate (20 mL) and washed with water (1×10 mL) and brine (1×20 mL). The organic layer was dried (MgSO₄) and concentrated in vacuo. The crude oil was purified via gravity feed chromatography using 60% ethyl acetate/hexane. The product was isolated as a clear oil (70 mg, 19% yield):

¹HNMR (400 MHz, CDCl₃) δ 8.602 (bs, 2H), 7.811(d, 1H), 4.825 (qd, 2H), 4.397 (q, 2H), 3.411 (bs, 1H), 3.035 (qd, 4H), 2.833 (t, 1H), 2.633 (m, 1H), 1.432 (t, 3H): m/z 300 (M+H).

### EXAMPLE 310

This example illustrates the production of 9-(morpholin-4-5,6,9,10-tetrahydro-7H-[1,4]oxazino[4′,3′:1,5]pyrazolo[3,4-f]isoquinolin-7-one.

Ethyl 2-(oxiran-2-ylmethyl)-4,5-dihydro-2H-pyrazolo[3,4-f]isoquinoline-3-carboxylate (1.34 mmol, 400 mg) was dissolved in acetonitrile (4 mL) and the morpholine (2.0 mmol, 175 mg, 175 μL) and the ytterbium triflate (0.2 mmol, 124 mg) were added. The vial was capped and heated to 70° C. for 4 h with stirring. After four hours of heating, the reaction vessel was allowed to cool and upon cooling the desired product precipitated and was collected via filtration. The product was isolated as a white solid (55 mg, 12% yield):

¹HNMR (400 MHz, CDCl₃) δ 8.538 (bs, 2H), 7.662 (d, 1H), 4.916 (m, 1H), 4.501(qd, 2H), 3.708 (bs, 4H), 3.178-3.079 (m, 2H), 2.995 (d, 2H), 2.872-2.803 (m, 2H), 2.606 (bs, 4H): m/z 341 (M+H). The following lactones were made (using the appropriate amine) in the same manor as 9-(morpholin-4-ylmethyl)-5,6,9,10-tetrahydro-7H-[1,4]oxazino[4′,3′:1,5] pyrazolo[3,4-f]isoquinolin-7-one:

### EXAMPLE 311

This example illustrates the production of 9-{[(2-thien-2-ylethyl)amino]-methyl}-5,6,9,10-tetrahydro-7H-[1,4]oxazino[4′,3′:1,5]pyrazolo[3,4-f]isoquinolin-7-one trifluoroacetate.

The product was isolated as a pale yellow solid (129 mg, 23% yield): ¹HNMR (400 MHz, DMSO) δ 8.750 (s 1H), 8.655(d, 1H), 7.985 (d, 1H), 7.337 (d, 1H), 6.936 (m, 2H), 4.358 (dd, 2H), 4.220 (dd, 2H), 3.82 (m, 1H), 3.582 (m, 1H), 3.460 (dd, 1H), 3.124 (t, 2H), 3.041 (m, 4H), 2.901 (t, 2H),: m/z 381 (M+H).

### EXAMPLE 312

This example illustrates the production for the production of 9-{[(4-aminocyclohexyl)amino]methyl}-5,6,9,10-tetrahydro-7H-[1,4]oxazino-[4′,3′:1,5]pyrazolo[3,4-f]isoquinolin-7-one trifluoroacetate.

The product was isolated as a tan solid (338 mg, 61% yield): ¹HNMR (400 MHz, DMSO) δ 8.682 (s, 1H), 8.612 (d, 1H), 7.880(d, 1H), 4.502 (dd, 2H), 3.454 (t, 2H), 3.381 (t, 2H), 2.979 (t, 2H), 1.941 (d, 4H), 1.326 (t, 4H): m/z 368 (M+H).

### EXAMPLE 313

This example illustrates the production of 9-(aminomethyl)-5,6,9,10-tetrahydro-7H-[1,4]oxazino[4′,3′:1,5]pyrazolo[3,4-f]isoquinolin-7-one trifluoroacetate.

The product was isolated as a yellow solid (85.5 mg, 21% yield): ¹HNMR (400 MHz, DMSO) δ 8.726 (s, 1H), 8.651 (d, 1-H), 7.968 (d, 1H), 4.588 (dd, 2H), 4.282-4.244 (3H), 3.430 (dd, 4H): m/z 271 (M+H).

### EXAMPLE 314

This example illustrates the production of lithium 2-(2-hydroxy-3-morpholin-4-ylpropyl)-4,5-dihydro-2H-pyrazolo[3,4-f]isoquinoline-3-carboxylate.

9-(morpholin-4-ylmethyl)-5,6,9,10-tetrahydro-7H-[1,4]oxazino-[4′,3′:1,5]pyrazolo[3,4-f]isoquinolin-7-one (0.41 mmol, 139.5 mg) was dissolved in 50% THF/water (4.0 mL) and lithium hydroxide (2M, 0.21 mL) was added. The reaction mixture stirred at 23° C. for 3hours. The sample was concentrated under nitrogen stream. The residue was triturated with acetonitrile and sonicated to produce a fine powder. The product was collected via filtration and isolated as a light tan solid (140.4 mg, 95% yield):

¹HNMR (400 MHz, DMSO) δ 8.468 (d, 2H), 7.655 (s, 1H), 7.544 (s, 1H), 4.601 (bs, 2H), 3.947 (m, 1H), 3.633 (s, 4H), 2.931-2.88 (d, 4H), 2.555 (m, 6H): m/z 359 (M+H).

The following compounds were hydrolyzed from the lactone to the acid in the same manner as lithium 2-(2-hydroxy-3-morpholin-4-ylpropyl)-4,5-dihydro-2H-pyrazolo[3,4-f]isoquinoline-3-carboxylate

### EXAMPLE 315

This example illustrates the production of 2-(2-hydroxy-3-piperazin-1-ylpropyl)-4,5-dihydro-2H-pyrazolo[3,4-f]isoquinoline-3-carboxylic acid dihydrochloride.

The product was isolated as a tan amorphous solid (254 mg, 95% yield):

¹HNMR (400 MHz, DMSO) δ 9.055 (d, 1H), 8.941 (s, 1H), 7.211 (d, 1H), 4.742 (m, 1H), 4.421 (dd, 2H), 3.380 (t, 2H), 3.251 (t, 2H), 2.875 (m, 4H), 2.587 (t, 2H), 2.232-2.172 (m, 4H): m/z 358 (M+H).

### EXAMPLE 316

This example illustrates the production of lithium 2-(2-hydroxy-3-piperidin-1-ylpropyl)-4,5-dihydro-2H-pyrazolo[3,4-f]isoquinoline-3-carboxylate.

The product was isolated as a dark tan solid (120.7 mg, 96% yield): ¹HNMR (400 MHz, DMSO) δ 9.068 (d, 1H), 8.946 (s, 1H), 7.222 (d, 1H), 4.499 (m, 2H), 4.321 (t, 1H), 3.661 (t, 2H), 3.551 (t, 2H), 2.553 (t, 2H), 2.321-2.130 (m, 4H), 1.431-1.256 (m, 6H): m/z 357 (M+H).

### EXAMPLE 317

This example illustrates the production of lithium 2-[2-hydroxy-3-(3-oxopiperazin-1-yl)propyl]-4,5-dihydro-2H-pyrazolo[3,4-f]isoquinoline-3-carboxylate.

The product was isolated as a tan solid (193.7 mg, 98% yield): ¹HNMR (400 MHz, DMSO) δ 9.063 (d, 1H), 8.941 (s, 1H), 7.218 (d, 1H), 4.605 (m, 1H), 4.492 (d, 1H), 4.326 (m, 1H), 3.661 (t, 2H), 3.390-3.223 (m, 2H), 3.055 (d, 1H), 2.723-2.500 (m, 5H), 1.921-1.756 (m, 2H): m/z 372 (M+H).

### EXAMPLE 318

This example illustrates the production of lithium 2-(2-hydroxy-3-pyrrolidin-1-ylpropyl)-4,5-dihydro-2H-pyrazolo[3,4-f]isoquinoline-3-carboxylate.

The product was isolated as a tan solid (104.8 mg, 92% yield): ¹HNMR (400 MHz, DMSO) δ 9.063 (d, 1H), 8.941 (s, 1H), 7.218 (d, 1H), 4.661-4.491 (m, 2H), 4.322 (t, 1H), 3.665 (t, 2H), 2.731-2.448 (m, 8H), 1.881-1.664 (m, 4H): m/z 343 (M+H).

### EXAMPLES 319-325

| | | | Isolated | Percent | Descrip- | (M + H) |
|---|---|---|---|---|---|---|
| EXAMPLE | Name | Lot # | Amount | Yield | tion | m/z |
| 319 | lithium 2-[2- | 11934-163-2 | 124.7 mg | 98% | Tan | 302 |
| | hydroxy-3- | | | | solid | |
| | (methylamino | | | | | |
| | propyl]-4,5- | | | | | |
| | dihydro-2H- | | | | | |
| | pyrazolo[3,4- | | | | | |
| | f]isoquinoline- | | | | | |
| | 3-carboxylate | | | | | |
| 320 | 2-[3-(4- | 11934-163-4 | 802.7 mg | 98% | Yellow | 372 |
| | aminopiperidi | | | | oil | |
| | n-1-yl)-2- | | | | | |
| | hydroxypropyl] | | | | | |
| | -4,5-dihydro-2H- | | | | | |
| | pyrazolo[3,4- | | | | | |
| | f]isoquinoline- | | | | | |
| | 3-carboxylic acid | | | | | |
| | bis(trifluoroac | | | | | |
| | etate) | | | | | |
| 321 | lithium 2-{3-[4- | 11934-163-5 | 342.6 mg | 95% | Tan | 399 |
| | (aminocarbonyl) | | | | solid | |
| | piperidin-1-yl]-2- | | | | | |
| | hydroxypropyl}-4,5- | | | | | |
| | dihydro-2H- | | | | | |
| | pyrazolo[3,4- | | | | | |
| | f]isoquinoline- | | | | | |
| | 3-carboxylate | | | | | |
| 322 | lithium 2-{3-[(4- | 11934-163-6 | 150.6 mg | 70% | Off | 385 |
| | aminocyclohexyl) | | | | white | |
| | amino]-2- | | | | solid | |
| | hydroxypropyl}-4,5- | | | | | |
| | dihydro-2H- | | | | | |
| | pyrazolo[3,4- | | | | | |
| | f]isoquinoline | | | | | |
| | 3-carboxylate | | | | | |
| 323 | lithium 2-{2- | 11934-163-8 | 201.5 mg | 97% | Off | 384 |
| | hydroxy-3- | | | | white | |
| | [(thien-2- | | | | solid | |
| | ylmethyl)amin | | | | | |
| | o]propyl}-4,5- | | | | | |
| | dihydro-2H- | | | | | |
| | pyrazolo[3,4- | | | | | |
| | f]isoquinoline- | | | | | |
| | 3-carboxylate | | | | | |
| 324 | lithium 2-{2- | 11934-163-9 | 85.0 mg | 95% | Off | 398 |
| | hydroxy-3-[(2- | | | | white | |
| | thien-2- | | | | solid | |
| | ylethyl)amino] | | | | | |
| | propyl}-4,5- | | | | | |
| | dihydro-2H- | | | | | |
| | pyrazolo[3,4- | | | | | |
| | f]isoquinoline- | | | | | |
| | 3-carboxylate | | | | | |
| 325 | lithium 2-[3- | 11934-163-10 | 216.5 mg | 99% | Tan | 404 |
| | (2,3-dihydro- | | | | solid | |
| | 1H-inden-2- | | | | | |
| | ylamino)-2- | | | | | |
| | hydroxypropyl]- | | | | | |
| | 4,5-dihydro-2H- | | | | | |
| | pyrazolo[3,4- | | | | | |
| | f]isoquinoline- | | | | | |
| | 3-carboxylate | | | | | |

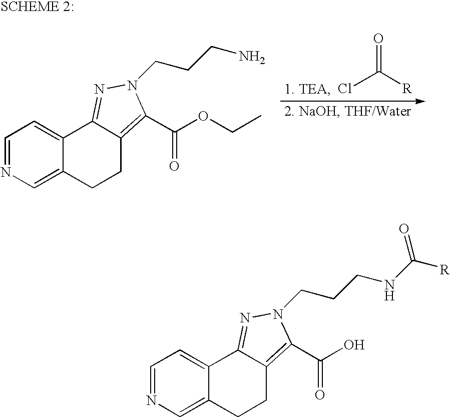

EXAMPLE 326.

This example illustrates the production of 2-[3-(2-furoylamino)propyl]-4,5-dihydro-2H-pyrazolo[3,4-f]isoquinoline-3-carboxylic acid trifluoroacetate.

Ethyl 2-(3-aminopropyl)-4,5-dihydro-2H-pyrazolo[3,4-f]isoquinoline-3-10 carboxylate hydrochloride (0.54 mmol, 200 mg) was suspended in DMF (3.0 mL) and triethylamine (2.16 mmol, 301 μL) was added. 2-Furoyl chloride (0.65 mmol, 64.1 μL) was added via syringe to the DMF solution in one portion. The reaction stirred at 23° C. for 2 hours. LC/MS indicates starting material is consumed and the desired M+H is seen for the ester. Sodium hydroxide (2.5 mmol, 100 mg) was added to the DMF solution and stirred at 23° C. for 4 hours. The DMF was removed in vacuo and the residue was reconstituted in 50% water/acetonitrile and acidified to pH=3 with trifluoroacetic acid and purified via reverse phase chromatography. The product was isolated as a white amorphous solid (76.9 mg, 49% yield): ¹HNMR (400 MHz, DMSO) δ 8.687 (s, 1H), 8.615 (d, 1H), 8.330 (t, 1H), 7.921 (m, 1H), 7.757 (m, 1H), 7.009 (dd, 1H), 6.560 (m, 1H), 4.609 (t, 2H), 3.244 (q, 2H), 2.099 (m, 2H): m/z 367.

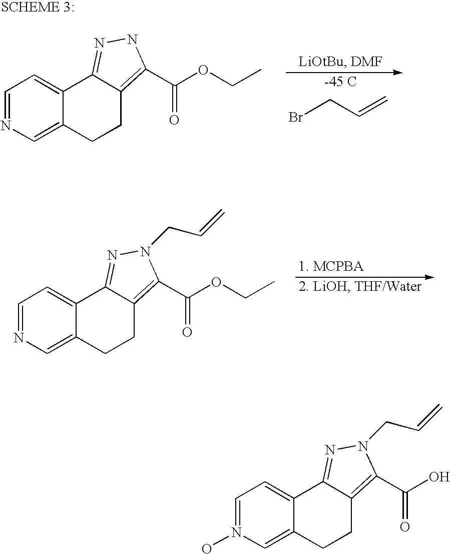

### EXAMPLE 327

This example illustrates the production of ethyl 2-allyl-4,5-dihydro-2H-pyrazolo[3,4-f]isoquinoline-3-carboxylate (allyl, ester). The pyrazole ester (1.23 mmol, 300 mg) was dissolved in DMF (10 mL) and cooled to −45° C. in a dry ice/acetonitrile bath. Lithium tert-butoxide (1M THF, 1.85 mmol, 1.85 mL) was added slowly being sure to maintain −45° C. The cold solution stirred for 1.5 h. Allyl bromide (1.85 mmol, 0.224 g) was added via syringe to the cold DMF solution and the ice bath was removed. The reaction was allowed to warm to 23° C. over 16 h. The crude reaction was diluted with ethyl acetate (20 mL) and washed with water (1×10 mL) and brine (1×20 mL). The organic layer was dried (MgSO₄) and concentrated in vacuo. The crude oil was purified via gravity feed chromatography using 60% ethyl acetate/hexane. The product was isolated as a clear oil (180 mg, 52% yield): ¹HNMR (400 MHz, CDCl₃) δ 8.480 (bs, 2H), 7.749 (d, 1H), 6.400-5.990 (m, 1H), 5.207 (dd, 2 h), 4.459-4.338 (m, 3H), 3.060 (t, 2H), 2.942 (t, 2H), 1.397 (t, 3H): m/z 284.

### EXAMPLE 328

Ethyl 2-(oxiran-2-ylmethyl)-4,5-dihydro-2H-pyrazolo[3,4-f]isoquinoline-3-carboxylate (Oxidation)-(ethyl 2-allyl-4,5-dihydro-2H-pyrazolo[3,4-f]isoquinoline-3-carboxylate) (0.64 mmol, 180 mg) was dissolved in dichloromethane (4.0 mL) and meta-chloro-per-benzoic acid (0.96 mmol, 195 mg, 57-86% purity) was added in one portion at 23° C. The reaction stirred for 30 minutes. LC/MS shows starting material is consumed and desired product M+H is evident. The organic layer was washed with water ((1×2 mL), saturated bicarbonate (2×2 mL) and brine (1×2 mL). The organic layer was dried (MgSO₄) and concentrated to dryness. Isolated 145 mg (76% yield) of crude product, used without further purification.

### EXAMPLE 329

This example illustrates the production of 2-allyl-4,5-dihydro-2H-pyrazolo[3,4-f]isoquinoline-3-carboxylic acid 7-oxide). (0.50 mmol, 145 mg) was dissolved in 50% THF/water (2.0 mL) and lithium hydroxide (1.0 mmol, 42 mg) was added. The reaction mixture stirred at 23° C. for 2 hours. LC/MS confirms reaction is done. The reaction was concentrated under nitrogen stream, reconstituted in water/acetonitrile and acidified to pH=3 using trifluoroacetic acid, then purified via reverse phase chromatography. The product was isolated as a pale pink solid (84.0 mg, 62% yield): ¹HNMR (400 MHz, DMSO) δ 8.209 (bs, 1H), 8.052 (d, 1H), 7.530 (d, 1H), 6.039-5.947 (m, 1H), 5.170 (d, 2H), 5.060 (qd, 2H), 2.950 (q, 2H), 2.850 (q, 2H): m/z 272 (M+H).

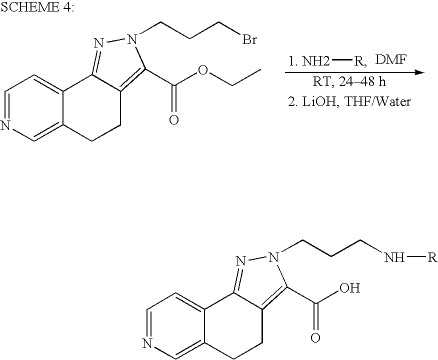

### EXAMPLE 330

This example illustrates the production of 2-(3-{[2-(2-chlorophenyl)ethyl]-amino}propyl)-4,5-dihydro-2H-pyrazolo[3,4-f]isoquinoline-3-carboxylic acid trifluoroacetate

The product was isolated as a white amorphous solid (416.9 mg, 92% yield):

¹HNMR (400 MHz, DMSO) δ 8.680 (d, 1H), 8.595 (d, 1H), 7.815 (bs, 1H), 7.430 (dd, 1H), 7.360-7.245 (m, 3H), 4.644 (t, 4H), 3.490 (m, 1H), 3.245 (m, 2H), 2.983 (m, 4H), 2.550 (m, 1H), 2.252 (t, 2H): m/z 411 (M+H).

### EXAMPLE 331

This example illustrates the production of 2-(3-{[2-(3-chlorophenyl)ethyl]-amino}propyl)-4,5-dihydro-2H-pyrazolo[3,4-f]isoquinoline-3-carboxylic acid trifluoroacetate

The product was isolated as a white amorphous solid (397.8 mg, 88% yield):

¹HNMR (400 MHz, DMSO) δ 8.689 (s, 1H), 8.614 (d, 2H), 7.820 (d, 1H), 7.326-7.295 (m, 2H), 7.200 (d, 1H), 4.636 (t, 4H), 3.165 (bs, 2H), 2.983 (m, 4H), 2.868 (t, 2H), 2.160 (t, 2H): m/z 411 (M+H).

### EXAMPLE 332

This example illustrates the production of 2-(3-{[2-(4-chlorophenyl)ethyl]amino}propyl)-4,5-dihydro-2H-pyrazolo[3,4-f]isoquinoline-3-carboxylic acid trifluoroacetate.

The product was isolated as a white amorphous solid (455.7 mg, 98% yield):

¹HNMR (400 MHz, DMSO) δ 8.680 (bs, 2H), 8.635 (d, 1H), 7.889 (d, 1H), 7.359 (d, 2H), 7.257 (d, 2H), 4.638 (t, 4H), 3.140 (m, 2H), 2.991(m, 4H), 2.851 (t, 2H), 2.160 (t, 2H): m/z 411 (M+H).

### EXAMPLE 333

This example illustrates the production of 2-{3-[(2-furylmethyl)amino]-propyl}-4,5-dihydro-2H-pyrazolo[3,4-f]isoquinoline-3-carboxylic acid trifluoroacetate.

The product was isolated as an off white amorphous solid (208.5 mg, 54% yield): ¹HNMR (400 MHz, DMSO) δ 8.712 (s, 1H), 8.639 (d, 1H), 7.860 (d, 1H), 7.724 (s, 1H), 6.545 (d, 1H), 6.467 (m, 1H), 4.628 (t, 2H), 4.220 (bs, 2H), 3.016 (s, 4H), 2.923 (bs, 2H), 2.139 (m, 2H): m/z 353 (M+H).

### EXAMPLE 334

This example illustrates the production of 2-{3-[(2-chlorobenzyl)amino]-propyl}-4,5-dihydro-2H-pyrazolo[3,4-f]isoquinoline-3-carboxylic acid trifluoro-acetate.

The product was isolated as a white amorphous solid (210.4 mg, 64% yield): —HNMR (400 MHz, DMSO) δ 8.777 (s, 1H), 8.713-(d, 1H), 7.929 (d, 1H), 7.630 (d, 2H), 7.461 (m, 2H), 4.717 (t, 4H), 4.325 (s, 2H), 3.019 (m, 4H), 2.264 (t, 2H): m/z 397 (M+H).

### EXAMPLE 335

This example illustrates the production of 2-{3-[(3-chlorobenzyl)amino]propyl}-4,5-dihydro-2H-pyrazolo[3,4-f]isoquinoline-3-carboxylic acid trifluoroacetate.

The product was isolated as a white amorphous solid (259 mg, 79% yield):

¹HNMR (400 MHz, DMSO) δ 8.769 (s, 1H), 8.702 (d, 1H), 7.912 (d, 1H), 7.604 (s, 1H), 7.477 (m, 3H), 4.702 (t, 4H), 4.206 (s, 2H), 3.052 (m, 4H), 2.264 (t, 2H): m/z 397 (M+H).

### EXAMPLE 336

This example illustrates the production of 2-{3-[(4-chlorobenzyl)amino]-propyl}-4,5-dihydro-2H-pyrazolo[3,4-f]isoquinoline-3-carboxylic acid trifluoroacetate.

The product was isolated as a white amorphous solid (250 mg, 77% yield):

¹HNMR (400 MHz, DMSO) δ 8.742 (s, 1H), 8.678 (d, 1H), 7.867(d, 1H), 7.510 (s, 4H), 4.690 (t, 4H), 4.189 (s, 2H), 3.063 (m, 4H), 2.214 (t, 2H): m/z 397 (M+H).

### EXAMPLE 337

This example illustrates the production of 2-(3-{[2-(4-bromophenyl)ethyl]-amino}propyl)-4,5-dihydro-2H-pyrazolo[3,4-f]isoquinoline-3-carboxylic acid trifluoroacetate.

The product was isolated as a white amorphous solid (345 mg, 92% yield):

¹HNMR (400 MHz, DMSO) δ 8.732 (s, 1H), 8.662 (d, 1H), 7.888-(d, 1H), 7:564 (d, 2H), 7.262 (d, 2H), 4.689 (t, 4H), 3.200 (bs, 2H), 3.063 (m, 4H), 2.884 (t, 2H), 2.214 (t, 2H): m/z 456 (M+H).

### EXAMPLE 338

This example illustrates the production of 2-{3-[(2,2,2-trifluoroethyl)amino]-propyl}-4,5-dihydro-2H-pyrazolo[3,4-f]isoquinoline-3-carboxylic acid trifluoroacetate.

The product was isolated as a white amorphous solid (213 mg, 73% yield):

¹HNMR (400 MHz, DMSO) δ 8.698 (s, 1H), 8.627 (d, 1H), 7.870 (d, 1H), 4.635 (t, 2H), 3.581 (q, 2H), 3.016 (s, 4H), 2.992 (t, 2H), 2.135 (t, 2H): m/z 355 (M+H).

### EXAMPLE 339

This example illustrates the production of 2-(3-{[2-(4-fluorophenyl) ethyl]amino}propyl)-4,5-dihydro-2H-pyrazolo[3,4-f]isoquinoline-3-carboxylic acid trifluoroacetate.

The product was isolated as a white amorphous solid (118 mg, 36% yield):

¹HNMR (400 MHz, DMSO) δ 8.716 (s, 1H), 8.644 (d, 1H), 7.886 (d, 1H), 7.278 (t, 2H), 7.133 (t, 2H), 4.655 (t, 2H), 3.139 (m, 2H), 3.001 (m, 6H), 2.861 (t, 2H), 2.172 (t, 2H): m/z 395 (M+H).

### EXAMPLE 340

This example illustrates the production of 2-(3-{[2-(4-methylphenyl)ethyl]amino}propyl)-4,5-dihydro-2H-pyrazolo[3,4-f]isoquinoline-3-carboxylic acid trifluoroacetate.

The product was isolated as a white amorphous solid (153 mg, 48% yield):

¹HNMR (400 MHz, DMSO) δ 8.715 (s, 1H), 8:642 (d, 1H), 7.886 (d, 1H), 7.110 (s, 4H), 4.655 (t, 2H), 3.129 (m, 2H), 3.031 (m, 6H), 2.816 (t, 2H), 2.249 (s, 3H), 2.171 (t, 2H): m/z 391 (M+H).

### EXAMPLE 341

This example illustrates the production of 2-(3-{[2-(4-ethylphenyl) ethyl]amino}propyl)-4,5-dihydro-2H-pyrazolo[3,4-f]isoquinoline-3-carboxylic acid trifluoroacetate.

The product was isolated as a white amorphous solid (137 mg, 41% yield):

¹HNMR (400 MHz, DMSO) δ 8.706 (s, 1H), 8.635 (d, 1H), 7.875 (d, 1H), 7.137 (s, 4H), 4.655 (t, 2H), 3.120 (m, 2H), 3.028 (m, 6H), 2.825 (t, 2H), 2.536 (q, 2H), 2.199 (t, 2H), 1.133 (t, 3H): m/z 405 (M+H).

### EXAMPLE 342

This example illustrates the production of 2-{3-[(2-phenylpropyl)amino]-propyl}-4,5-dihydro-2H-pyrazolo[3,4-f]isoquinoline-3-carboxylic acid trifluoroacetate.

The product was isolated as a white amorphous solid (168 mg, 52% yield):

¹HNMR (400 MHz, DMSO) δ 8.707 (s, 1H), 8.636 (d, 1H0, 7.859 (d, 1H), 7.270 (m, 5H), 4.627 (t, 2H), 3.145 (m, 2H), 3.105 (m, 1H), 3.026 (s, 4H), 2.935 (m, 2H), 2.158 (m, 2H0, 1.241 (d, 3H): m/z 391 (M+H).

### EXAMPLE 343

This example illustrates the production of 2-(3-{[2-(4-chlorophenyl)propyl]-amino}propyl)-4,5-dihydro-2H-pyrazolo[3,4-f]isoquinoline-3-carboxylic acid trifluoroacetate.

The product was isolated as a white amorphous solid (35 mg, 10% yield):

¹HNMR (400 MHz, DMSO) δ 8.670 (s, 1H), 8.604 (d, 1H), 7.802 (d, 1H), 7.342 (q (a,b), 4H), 4.618 (t, 2H), 3.140 (m, 2H), 3.087 (m, 1H), 3.011 (s, 4H), 2.930 (m, 2H), 2.142 (t, 2H), 1.220 (d, 3H): m/z 425 (M+H).

### EXAMPLE 344

This example illustrates the production of 2-{3-[(3,3-diphenylpropyl)amino]propyl}-4,5-dihydro-2H-pyrazolo[3,4-f]isoquinoline-3-carboxylic acid trifluoroacetate.

The product was isolated as a white amorphous solid (253 mg, 66% yield):

¹HNMR (400 MHz, DMSO) δ 8.671 (s, 1H), 8.599 (d, 1H), 7.799 (d, 1H), 7.252 (m, 7H), 7.155(m, 3H), 4.618 (t, 2H), 4.014 (t, 1H), 3.006 (s, 4H), 2.951 (m, 2H), 2.784 (m, 2H), 2.319 (m, 2H), 2.104 (m, 2H): m/z 467 (M+H).

### EXAMPLE 345

This example illustrates the production of ethyl 2-{2-[(tert-butoxycarbonyl)amino]ethyl}-4,5-dihydro-2H-pyrazolo[3,4-f]isoquinoline-3-carboxylate.

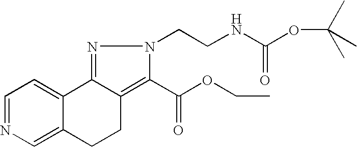

To a cooled (0° C.) solution of ethyl 4,5-dihydro-2H-pyrazolo[3,4-f]isoquinoline-3-carboxylate (2.00 g, 8.22 mmol) in anhydrous DMF (100 mL) was added lithium t-butoxide (1M in THF, 12.3 mL) dropwise. The reaction stirred for 30 min, then a solution of tert-butyl 2-bromoethylcarbamate [tert-butyl 2-bromoethylcarbamate] (2.75 g, 12.3 mmol,) and sodium iodide (1.84 g, 12.3 mmol) in anhydrous DMF (10 mL) was added dropwise. The reaction was allowed to stir and warm to room temperature for 26 h. The reaction was poured into water and brine, extracted with ethyl acetate, dried over MgSO₄, and concentrated. The brown residue was chromatographed (4% ethanol/CH₂Cl₂). The appropriate fractions were concentrated to a yellow solid, then rinsed with diethyl ether to yield a white powder (0.778 g, 24.6% yield): mp 130.6-131.9° C.; ¹H NMR (DMSO-d₆/300 MHz) ? 1.32 (s, 9H), 1.39 (t, 3H), 2.94-3.02 (m, 4H), 3.37 (m, 2H), 4.37 (q, 2H), 4.60 (t, 2H), 6.93 (t, 1H), 7.61 (d, 1H), 8.49 (d, 1H), 8.52 (s, 1H); HRMS calcd for (M+H) 387.2027, found 387.2011.

### EXAMPLE 346

This example illustrates the production of lithium 2-{2-[(tert-butoxycarbonyl)amino]ethyl}-4,5-dihydro-2H-pyrazolo[3,4-f]-isoquinoline-3-carboxylate

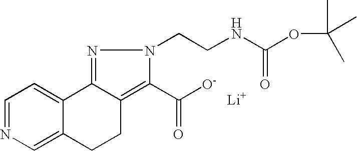

A solution of ethyl 2-{2-[(tert-butoxycarbonyl)amino]ethyl}-4,5-dihydro-2H-pyrazolo[3,4-f]isoquinoline-3-carboxylate (0.701 g, 1.81 mmol) and LiOH.H₂O (0.235 g, 5.60 mmol) in 40 mL of THF/H₂O (1:1) was stirred at room temperature for 1 h. The reaction mixture was concentrated to a white solid which was used in the next step without further purification (0.653 g, quantitative yield): mp>300° C.; ¹H NMR (DMSO-d₆/300 MHz) ??1.37 (s, 9H), 2.83-2.99 (m, 4H), 3.43 (m, 2H), 4.65 (t, 2H), 7.53 (d, 1H), 7.74 (br s, 1H), 8.41 (d, 1H), 8.45 (s, 1H); HRMS calcd for (M+H) 359.1714, found 359.1700.

### EXAMPLE 347

This example illustrates the production of 2-(2-aminoethyl)-4,5-dihydro-2H-pyrazolo[3,4-f]isoquinoline-3-carboxylic acid dihydrochloride.

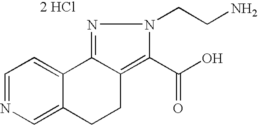

To a flask charged with lithium 2-{2-[(tert-butoxycarbonyl)amino]ethyl}-4,5-dihydro-2H-pyrazolo[3,4-f]isoquinoline-3-carboxylate (0.520 g, 1.43 mmol) was added 4N HCl/dioxane. After 1 h the reaction mixture was concentrated to a white solid (0.457 g, 96.4% yield): mp 266.6-270.9° C.; ¹H NMR (DMSO-d₆/300 MHz) ? 3.09-3.11 (m, 4H), 3.34 (m, 2H), 4.91 (t, 2H), 8.16 (d, 1H), 8.60 (br s, 3H), 8.74 (d, 1H), 8.86 (s, 1H); HRMS calcd for (M+H) 259.1190, found 259.1173.

### EXAMPLE 348

This example illustrates the production of ethyl 2-(2-aminoethyl)-4,5-dihydro-2H-pyrazolo[3,4-f]isoquinoline-3-carboxylate dihydrochloride.

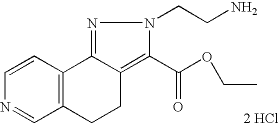

To a flask charged with ethyl 2-{2-[(tert-butoxycarbonyl)amino]ethyl}-4,5-dihydro-2H-pyrazolo[3,4-f]isoquinoline-3-carboxylate (3.90 g, 10.09 mmol) was added 4N HCl/dioxane. After 1 h the reaction mixture was concentrated to a white solid (3.580 g, 91.4% yield): mp 220.7-223.7° C.; ¹H NMR (DMSO-d₆/300 MHz) 1.40 (t, 3H), 3.10-3.19 (m, 4H), 3.41-3.36 (m, 2H), 4.41 (q, 2H), 4.91 (t, 2H), 8.24 (d, 1H), 8.45 (br s, 3H), 8.81 (d, 1H), 8.92 (s, 1H); HRMS calcd for (M+H) 287.1503, found 287.1502.

### EXAMPLE 349

This example illustrates the production of 5,6,9,10-tetrahydropyrazino[1′,2′:1,5]pyrazolo[3,4-f]isoquinolin-7(8H)-one.

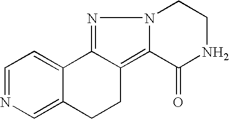

To a flask charged with ethyl 2-(2-aminoethyl)-4,5-dihydro-2H-pyrazolo[3,4-f]isoquinoline-3-carboxylate dihydrochloride (1.005 g, 2.80 mmol) was added NH₄OH (20 mL) and ethanol (10 mL). After 5 min a white precipitate formed. The reaction was filtered, and the white solid rinsed with water, ethanol, and diethyl ether to yield the desired lactam (0.653 g, 97.1% yield): mp>300° C.;

¹H NMR (DMSO-d₆/300 MHz) 2.94-3.01 (m, 4H), 3.64-3.68 (m, 2H), 4.39 (t, 2H), 7.60 (d, 1H), 8.28 (br s, 1H), 8.49 (d, 1H), 8.53 (s, 1H); HRMS calcd for (M+H) 241.1084, found 241.1082.

### EXAMPLE 350

This example illustrates the production of ethyl 2-(3-cyanopropyl)-4,5-dihydro-2H-pyrazolo[3,4-f]isoquinoline-3-carboxylate

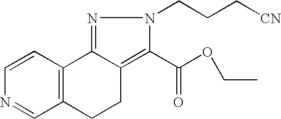

To a cooled (0° C.) solution of ethyl 4,5-dihydro-2H-pyrazolo[3,4-f]isoquinoline-3-carboxylate (5.60 g, 23.0 mmol) in anhydrous DMF (200 mL) was added lithium t-butoxide (1M in THF, 34.5 mL) dropwise. The reaction stirred for 30 min, then a solution of 4-bromo-butyronitrile (5.11 g, 34.5 mmol) and sodium iodide (5.18 g, 34.5 mmol) in anhydrous DMF (15 mL) was added dropwise. The reaction was allowed to warm to ambient temperature. After 2.5 h, the reaction was poured into water and brine, extracted with ethyl acetate, dried over MgSO₄, and concentrated. The brown residue was passed thru a silica plug, eluting with CH₂Cl₂. The eluant was concentrated, triturated with diethyl ether, and filtered to give the alkylated product as a tan solid (4.5869, 64.3% yield): mp 100.1-102.5° C.; ¹H NMR (DMSO-d₆/300 MHz) 1.38 (t, 3H), 2.15 (dd, 2H), 2.59 (t, 2H), 2.94-3.06 (m, 4H), 4.38 (q, 2H), 4.65 (t, 2H), 7.62 (d, 1H), 8.50 (d, 1H), 8.54 (s, 1H); HRMS calcd for (M+H) 311.1503, found 311.1462.

### EXAMPLE 351

This example illustrates the production of 2-(3-cyanopropyl)-4,5-dihydro, 2H-pyrazolo[3,4-f]isoquinoline-3-carboxylic acid

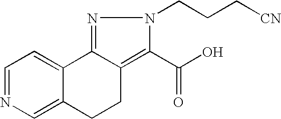

A solution of ethyl 2-(3-cyanopropyl)-4,5-dihydro-2H-pyrazolo[3,4-f]isoquinoline-3-carboxylate (0.339 g, 1.09 mmol) and LiOH.H₂O (0.056 g, 1.33 mmol) in 25 mL of THF/H₂O (1:1) was stirred at room temperature for 1.5 h. The reaction mixture was concentrated to the aqueous layer, and 3N HCl was added until a precipitate was formed (pH=6). The suspension was filtered and the solid dried in a vacuum oven overnight to yield an off-white solid (0.218 g, 70.8% yield): mp 267.7-267.8° C.; ¹H NMR (DMSO-d₆/300 MHz) 2.15 (dd, 2H), 2.58 (t, 2H), 2.95-3.03 (m, 4H), 4.66 (t, 2H), 7.62 (d, 1H), 8.49 (d, 1H), 8.53 (s, 1H); HRMS calcd for (M+H) 283.1190, found 283.1193.

### EXAMPLE 352

This example illustrates the production of ethyl 2-(4-aminobutyl)-4,5-dihydro-2H-pyrazolo[3,4-f]isoquinoline-3-carboxylate.

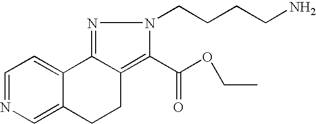

A pressure tube was charged with ethyl 2-(3-cyanopropyl)-4,5-dihydro-2H-pyrazolo[3,4-f]isoquinoline-3-carboxylate (1.001 g, 3.23 mmol) in ethanol (30 mL). The solution was saturated with NH₃ (g), and then approximately 1 mL of Raney Ni (50% by weight slurry in water) was added. The system was charged with H₂ (55 psi) and stirred for 7 days. The reaction mixture was filtered through Celite, rinsed with ethanol, and the filtrate concentrated to a yellow oil (0.820 g, 80.1% yield) which was used in the next reaction without further purification: ¹H NMR (DMSO-d₆/300 MHz) 1.34-1.41 (m, 5H), 1.83 (m, 2H), 2.57 (t, 2H), 2.94-3.04 (m, 4H), 4.37 (q, 2H), 4.56 (t, 2H), 7.61 (d, 1H), 8.48 (d, 1H), 8.53 (s, 1H); HRMS calcd for (M+H) 315.1816, found 315.1802.

### EXAMPLE 353

This example illustrates the production of 2-(4-aminobutyl)-4,5-dihydro-2H-pyrazolo[3,4-f]isoquinoline-3-carboxylic acid dihydrochloride.

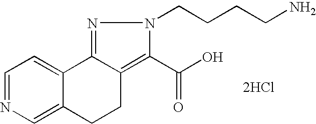

A solution of ethyl 2-(4-aminobutyl)-4,5-dihydro-2H-pyrazolo[3,4-f]isoquinoline-3-carboxylate (0.354 g, 1.13 mmol) and LiOH.H₂O (0.142 g, 3.38 mmol) in 40 mL of THF/H₂O (1:1) was stirred at room temperature for 1 h. The reaction mixture was concentrated to the aqueous layer and purified on a Gilson Reverse Phase High Performance Liquid Chromatography (RP HPLC) apparatus (5-95% acetonitrile/water). The appropriate fractions were concentrated. To this TFA salt residue was added 4N HCl/dioxane and methanol. After 1 h the suspension was filtered and the solid rinsed with diethyl ether to yield a yellow solid (0.0443 g, 10.9% yield): mp 255.2-260.3° C.; ¹H NMR (DMSO-d₆/300 MHz) 1.59-1.64 (m, 2H), 1.89-1.94 (m, 2H), 2.82 (m, 2H), 3.09-3.13 (m, 4H), 4.67 (t, 2H), 8.12-8.15 (m, 3H), 8.76 (d, 1H), 8.90 (s, 1H); HRMS calcd for (M+H) 287.1503, found 287.1483.

### EXAMPLE 354

This example illustrates the production of 2-(4-amino-4-oxobutyl)-4,5-dihydro-2H-pyrazolo[3,4-f]isoquinoline-3-carboxylic acid trifluoroacetate.

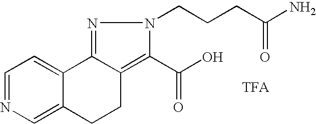

2-(3-cyanopropyl)-4,5-dihydro-2H-pyrazolo[3,4-f]isoquinoline-3-carboxylic acid (0.501 g, 1.77 mmol) was dissolved in conc. H₂SO₄ (5 mL) and heated on a steam bath for 0.5 h. Ice was then added and the solution brought to pH 3.5, at which point a precipitate formed. The solid was a mixture of the amide and acid. This mixture was purified on the Gilson RP HPLC (5-95% acetonitrile/water). The fractions corresponding to the amide were concentrated to a pale yellow solid (0.201 g, 28.6% yield): mp 201.6-206.7° C.; ¹H NMR (DMSO-d₆/300 MHz)???2.04-2.17 (m, 4H), 3.09-3.18 (m, 4H), 4.65 (t, 2H), 6.82 (br s, 1H), 7.35 (br s, 1H), 8.19 (d, 1H), 8.79 (d, 1H), 8.89 (s, 1H); HRMS calcd for (M+H) 301.1295, found 301.1274.

### EXAMPLE 355

This example illustrates the production of 2-(3-carboxypropyl)-4,5-dihydro-2H-pyrazolo[3,4-f]isoquinoline-3-carboxylic acid trifluoroacetate.

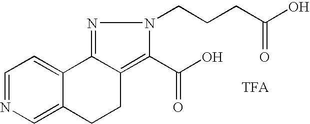

The fractions corresponding to the acid in example 2-(4-amino-4-oxobutyl)-4,5-dihydro-2H-pyrazolo[3,4-f]isoquinoline-3-carboxylic acid trifluoroacetate were concentrated to a yellow solid. HRMS calcd for (M+H) 302.1135, found 302.1147.

### EXAMPLE 356

This example illustrates the production of 2-{4-[bis(2-ethylbutyl)amino]butyl}-4,5-dihydro-2H-pyrazolo[3,4-f]isoquinoline-3-carboxylic acid hydrochloride.

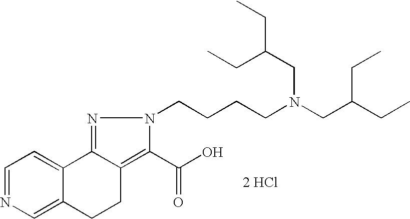

2-{4-[bis(2-ethylbutyl)amino]butyl}-4,5-dihydro-2H-pyrazolo[3,4-f]isoquinoline-3-carboxylic acid hydrochloride was obtained following example lithium 2-{4-[(2-ethylbutyl)amino]butyl}-4,5-dihydro-2H-pyrazolo[3,4-f]isoquinoline-3-carboxylate, but using 12.7 eq of 2-ethylbutyraldehyde. HRMS calcd for (M+H) 455.3381, found 455.3363.

### EXAMPLE 357

This example illustrates the production of 2-{2-[(2-ethylbutyl)amino]ethyl}-4,5-dihydro-2H-pyrazolo[3,4-f]isoquinoline-3-carboxylic acid.

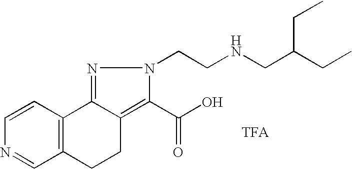

ethyl 2-(2-aminoethyl)-4,5-dihydro-2H-pyrazolo[3,4-f]isoquinoline-3-carboxylate dihydrochloride (0.502 g, 1.40 mmol) was neutralized by stirring with morpholino-methylpolystyrene resin (2.40 g, ˜3.5 mmol base/g resin) in CH₂Cl₂/methanol (20:1) for 0.5 h. The resin was filtered, rinsed with methanol, and the filtrates concentrated. The resulting white residue was dissolved in CH₂Cl₂/methanol (20:1). Six drops of glacial acetic acid were added with stirring, followed by 2-ethylbutyraldehyde (225 uL, 1.68 mmol). After 5 min, sodium triacetoxyborohydride (0.596 g, 2.80 mmol) was added. The reaction stirred overnight, both the mono- and dialkylated products formed. The reaction was quenched with water and extracted with CH₂Cl₂. The organic layers were concentrated and purified by Gilson RP HPLC (5-95% acetonitrile/water). The fractions corresponding to the monoalkylated product were concentrated. The residue was subjected to hydrolysis conditions [3 eq LiOH.H₂O, THF/H₂O (1:1)] for 18 h. The resulting product mixture was purified by Gilson RP HPLC (5-95% acetonitrile/water) and the appropriate fractions concentrated to a white solid (0.141 g, 20.6% yield): mp 200.3-202.4° C.; ¹H NMR (DMSO-d₆/300 MHz) 0.87 (t, 6H), 1.32-1.43 (m, 4H), 1.60-1.64 (m, 1H), 2.93-3.07 (m, 6H), 3.51 (t, 2H), 4.91 (t, 2H), 7.63 (d, 1H), 8.52 (d, 1H), 8.57 (s, 1H), 8.70 (br s, 1H); HRMS calcd for (M+H) 343.2129, found 343.2131.

### EXAMPLE 358

This example illustrates the production of 2-{2-[bis(2-ethylbutyl)amino]ethyl}-4,5-dihydro-2H-pyrazolo[3,4-f]isoquinoline-3-carboxylic acid dihydrochloride

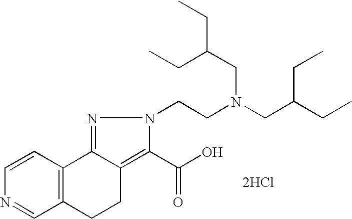

The fractions corresponding to the dialkylated product in the example which illustrates the production of 2-{2-[(2-ethylbutyl)amino]ethyl}-4,5-dihydro-2H-pyrazolo[3,4-f]isoquinoline-3-carboxylic acid, were concentrated to a white solid. HRMS calcd for (M+H) 427.3068, found 427.3083.

### EXAMPLE 359

This example illustrates the production of lithium 2-{4-[(2-ethylbutyl)amino]butyl}-4,5-dihydro-2H-pyrazolo[3,4-f]isoquinoline-3-carboxylate.

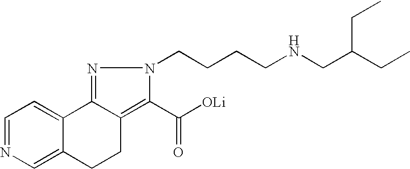

Synthesis was performed as in the example for the production of 2-{2-[(2-ethylbutyl)amino]ethyl}-4,5-dihydro-2H-pyrazolo[3,4-f]isoquinoline-3-carboxylic acid (omitting the initial neutralization step) using ethyl 2-(4-aminobutyl)-4,5-dihydro-2H-pyrazolo[3,4-f]isoquinoline-3-carboxylate (0.471 g, 1.5 mmol), 2-ethylbutyraldehyde (241 uL, 1.80 mmol), and sodium triacetoxyborohydride (0.636 g, 3.00 mmol). The reaction was monitored by LC-MS and stopped after 2 h. The residue obtained from the chromatographic purification was subjected to hydrolysis conditions [3 eq LiOH.H₂O, THF/H₂O (1:1)] for 18 h. The reaction mixture was concentrated and triturated with diethyl ether to yield a white solid (0.299 g, 52.7% yield): mp 125.1-128.7° C.; ¹H NMR (DMSO-d₆/300 MHz) 0.83 (t, 6H), 1.26-1.42 (m, 8H), 1.76-1.80 (m, 3H), 2.35-2.39 (m, 2H), 2.81-2.99 (m, 4H), 4.65 (t, 2H), 7.52 (d, 1H), 8.39 (d, 1H), 8.44 (s, 1H); HRMS calcd for (M+H) 371.2442, found 371.2458.

### EXAMPLE 360

This example illustrates the production of ethyl 6-benzyl-1-[4-(methylsulfonyl)-phenyl]-1,4,5,6-tetrahydropyrazolo[3,4-e]indazole-3-carboxylate.

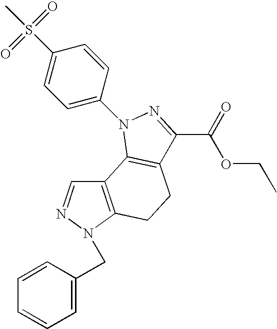

To N,N-dimethylacetamide dimethylacetal (250 mL) was added 1,3-cyclohexanedione (100 g, 0.89 mol). Contents were refluxed 2 hours, allowed to cool and concentrated in vacuo leaving an amber solid. After recrystallizing twice from EtOAc, the desired eneamine intermediate was obtained as light amber crystals, 64.2 g. The eneamine (21.7 g, 0.13 mol) and benzylhydrazine hydrochloride (25.5 g, 0.13 mol) were mixed in glacial acetic acid (200 mL) and stirred overnight. Contents were diluted with water, extracted with EtOAc, dried over MgSO₄, and concentrated in vacuo leaving the desired tetralone intermediate as an amber oil, 37.2 g. The tetralone was reacted with diethyl oxalate according to the procedure for the production of ethyl (7-hydroxy-1-oxo-1,2,3,4-tetrahydronaphthalen-2-yl)(oxo)acetate, to give the desired diketoester intermediate as an amber oil. The diketoester was reacted with 4-methylsulfonylphenylhydrazine according to the procedure for the production of ethyl 8-hydroxy-4,5-dihydro-2H-benzo[g]indazole-3-carboxylate, to give the desired product as white crystals (53% yield). FABHRMS m/z 477.1587 (M+H, C₂₅H₂₅N₄O₄S requires 477.1597).

Anal. Calcd for C₂₄H₂₄N₄O₄S: C, 63.01; H, 5.08; N, 11.76. Found: C, 62.93; H, 4.92; N, 11.77.

### EXAMPLE 361

This example illustrates the production of ethyl 1-[4-(methylsulfonyl)phenyl]-1,4,5,6-tetrahydropyrazolo[3,4-e]indazole-3-carboxylate.

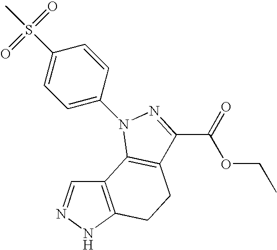

Ethyl 6-benzyl-1-[4-(methylsulfonyl)phenyl]-1,4,5,6-tetrahydropyrazolo[3,4-e]indazole-3-carboxylate (2.5 g, 0.005 mol), Pearleman's catalyst (1.0 g) and glacial acetic acid (30 mL) were shaken at 55 psi H₂ and 55° C., overnight. Contents were allowed to cool and filtered through clay. The filtrate was concentrated in vacuo and the residue was triturated with methanol and filtered to give the desired product as a white solid, 609 mg (32% yield). FABHRMS m/z 387.1161 (M+H, C₁₈H₁₉N₄O₄S requires 387.1127).

Anal. Calcd for C₁₈H₁₈N₄O₄S (0.8H₂O): C, 53.94; H, 4.93; N, 13.98. Found: C, 53.97; H, 4.71; N, 13.71.

### EXAMPLE 362

This example illustrates the production of 1-[4-(methylsulfonyl)phenyl]-1,4,5,6-tetrahydropyrazolo[3,4-e]indazole-3-carboxamide.

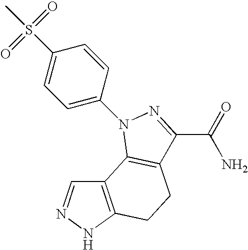

Ethyl 1-[4-(methylsulfonyl)phenyl]-1,4,5,6-tetrahydropyrazolo[3,4-e]indazole-3-carboxylate was reacted according to the procedure for the production of 8-hydroxy-4,5-dihydro-2H-benzo[g]indazole-3-carboxamide to give the desired product as a white solid (58% yield).

Anal. Calcd for C₁₆H₁₅N₅O₃S (0.5H₂O): C, 52.45; H, 4.40; N, 19.11. Found: C, 52.42; H, 4.41; N, 18.63.

### EXAMPLE 363

This example illustrates the production of 1,5,6,7-tetrahydro-4H-indazol-4-one.

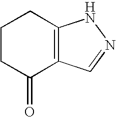

To N,N-dimethylacetamide dimethylacetal (250 mL) was added 1,3-cyclohexanedione (100 g, 0.89 mol). Contents were refluxed 2 hours, allowed to cool and concentrated in vacuo leaving amber solid. After recrystallizing twice from EtOAc, the desired eneamine intermediate was obtained as light amber crystals, 64.2 g. The eneamine (5.0 g, 0.03 mol) and p-tosylhydrazine hydrochloride (5.6 g, 0.3 mol) were stirred in methanol overnight. 4N HCl in dioxane (100 mL) was added and contents were stirred overnight. A white solid was filtered to give the desired product, 4.5 g (87% yield). FABHRMS m/z 137.0738 (M+H, C₇H N₂O requires 137.0715). ¹H NMR (DMSO-d₆/300 MHz): 7.90 (s, 1H); 2.90-2.80 (m, 2H); 2.40-2.30 (m, 2H); 2.10-2.00 (m, 3H).

Anal. Calcd for C₇H₈N₂O (0.05H₂O): C, 61.35; H, 5.96; N, 20.44. Found: C, 61.25; H, 5.98; N, 20.53.

### EXAMPLE 364

This example illustrates the production of 2-trityl-2,5,6,7-tetrahydro-4H-indazol-4-one.

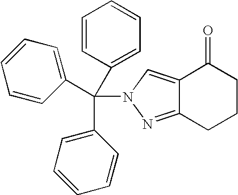

To 1,5,6,7-tetrahydro-4H-indazol-4-one (2.0 g, 0.012 mol) and triethylamine (4.4 mL, 0.031 mol) in CH₂Cl₂ (25 mL) was added dropwise trityl chloride (4.3 g, 0.0155 mol) in CH₂Cl₂ (20 mL). Contents were stirred overnight, washed with water and the CH₂Cl₂ layer was concentrated in vacuo. The residue was partitioned between ether and water. The ether layer was washed with brine, dried over MgSO₄ and concentrated in vacuo leaving an amber oil. Trituration with hexanes gave the desired product as a white solid, 3.6 g (79% yield). FABHRMS m/z 401.1608 (M+H, C₂₆H₂₃N₂O requires 401.1630). ¹H NMR (CDCl₃/300 MHz): 7.90 (s, 1H); 7.40-7.10 (m, 15H); 2.90-2.80 (m, 2H); 2.55-2.45 (m, 2H); 2.20-2.10 (m, 2H).

Anal. Calcd for C₂₆H₂₂N₂O: C, 82.51; H, 5.86; N, 7.4. Found: C, 82.36; H, 6.10; N, 7.32.

### EXAMPLE 365

This example illustrates the production of 6-methyl-2,4,5,6-tetrahydropyrazolo-[3,4-e]indazole-3-carboxamide.

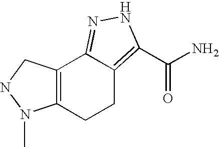

FABHRMS m/z 218.1051 (M+H, C₁₀H₁₂N₅O requires 218.1042).

### EXAMPLE 366

This example illustrates the production of ethyl 2-trityl-4-oxo-4,5,6,7-tetrahydro-2H-indazol-5-yl](oxo)acetate.

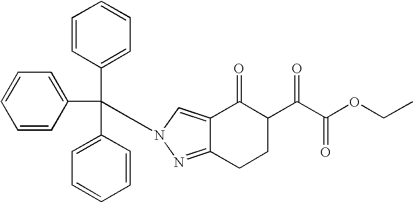

1-trityl-1,5,6,7-tetrahydro-4H-indazol-4-one- or 1-trityl-1,5,6,7-tetrahydro-4H-indazol-4-one was reacted according to the procedure for the production of ethyl (7-hydroxy-1-oxo-1,2,3,4-tetrahydronaphthalen-2-yl)(oxo)acetate to give the desired product as yellow crystals (82% yield). FABHRMS m/z 479.1971 (M+H, C₃₀H₂₇N₂O₄ requires 479.1965). ¹H NMR (CDCl₃/300 MHz): 7.97 (s, 1H); 7.40-7.10 (m, 15H); 4.40 (q, 2H); 3.15-3.05 (m, 2H); 2.95-2.85 (m, 2H); 1.45 (t, 3H).

Anal. Calcd for C₃₀H₂₆N₂O₄: C, 75.30; H, 5.48; N, 5.85. Found: C, 75.11; H, 5.38; N, 5.73.

### EXAMPLE 367

This example illustrates the production of ethyl 2,4,5,6-tetrahydropyrazolo[3,4-e]indazole-3-carboxylate.

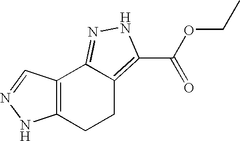

Ethyl oxo(4-oxo-1-trityl-4,5,6,7-tetrahydro-1H-indazol-5-yl)acetate- or ethyl oxo(4-oxo-2-trityl-4,5,6,7-tetrahydro-2H-indazol-5-yl)acetate or ethyl oxo(4-oxo-2-trityl-4,5,6,7-tetrahydro-2H-indazol-5-yl)acetate was reacted according to the procedure for the production of ethyl 8-hydroxy-4,5-dihydro-2H-benzo[g]indazole-3-carboxylate at 70° C. to give the desired product (67% yield). ¹H NMR (DMSO-d₆/300 MHz): 7.78 (s, 1H); 4.30 (q, 2H); 3.05-2.95 (m, 2H); 2.90-2.80 (m, 2H); 1.35 (t, 3H).

Anal. Calcd for C₁₁H₁₃N₄O₂ (2HOAc): C, 51.13; H, 5.72; N, 15.90. Found: C, 51.09; H, 5.80; N, 16.09.

### EXAMPLE 368

This example illustrates the production of 2,4,5,6-tetrahydropyrazolo[3,4-e]indazole-3-carboxamide.

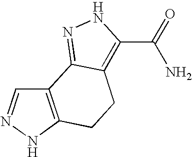

Ethyl 2,4,5,6-tetrahydropyrazolo[3,4-e]indazole-3-carboxylate was reacted according to the procedure for the production of 8-hydroxy-4,5-dihydro-2H-benzo[g]indazole-3-carboxamide to give the desired as an off-white solid (97% yield). FABHRMS m/z 204.0877 (M+H, C₉H₁₀N₅O requires 204.0885).

¹H NMR (DMSO-d₆+TFA/300 MHz): 7.85 (s, 1H); 7.30 (br s, 2H); 3.05-2.95 (m, 2H); 2.90-2.80 (m, 2H).

Anal. Calcd for C₉H₉N₅O: C, 53.20; H, 4.46; N, 34.47. Found: C, 53.14; H, 4.47; N, 34.57.

### EXAMPLE 369

This example illustrates the production of ethyl [1-(2-hydroxyethyl)-4-oxo-4,5,6,7-tetrahydro-1H-indazol-5-yl](oxo)acetate.

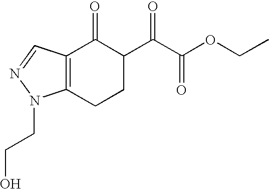

To N,N-dimethylacetamide dimethylacetal (250 mL) was added 1,3-cyclohexanedione (100 g, 0.89 mol). Contents were refluxed 2 hours, allowed to cool and concentrated in vacuo leaving amber solid. After recrystallizing twice from EtOAc, the desired eneamine intermediate was obtained as light amber crystals, 64.2 g. The eneamine (20.0 g, 0.12 mol) and hydroxyethylhydrazine (7.2 g, 0.12 mol) were mixed in methanol (50 mL) and stirred 72 hours. Contents were concentrated in vacuo leaving an oil, 13.4 g which was purified by silica gel chromatography eluting with 5% MeOH/EtOAc to give the desired tetralone intermediate as a yellow solid, 17.6 g (81% yield). The tetralone was reacted according to the procedure for the production of ethyl (7-hydroxy-1-oxo-1,2,3,4-tetrahydronaphthalen-2-yl)(oxo)acetate to give the desired product as a white solid (38% yield). FABHRMS m/z 281.1148 (M+H, C₁₃H₁₇N₂O₅ requires 281.1137).

Anal. Calcd for C₁₃H₁₆N₂O₅: C, 55.71; H, 5.75; N, 9.99. Found: C, 55.63; H, 5.67; N, 9.96.

### EXAMPLE 370

This example illustrates the production of ethyl 6-(2-hydroxyethyl)-2,4,5,6-tetrahydropyrazolo[3,4-e]indazole-3-carboxylate acetate

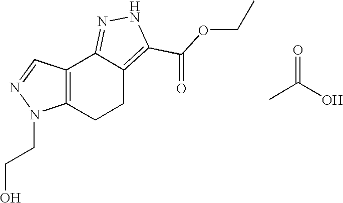

Ethyl 6-(2-hydroxyethyl)-2,4,5,6-tetrahydropyrazolo[3,4-e]indazole-3-carboxylate acetate was prepared according to the procedure for the production of ethyl 8-hydroxy-4,5-dihydro-2H-benzo[g]indazole-3-carboxylate to give the desired product as a white solid (80% yield). FABHRMS m/z 277.1296 (M+H, C₁₃H₁₇N₄O₃ requires 277.1301).

Anal. Calcd. for C₁₃H₁₆N₄O₃ (HOAc):C, 53.56; H, 5.99; N, 16.66. Found: C, 53.45; H, 5.71; N, 16.60.

### EXAMPLE 371

This example illustrates the production of 6-(2-hydroxyethyl)-2,4,5,6-tetrahydro-pyrazolo[3,4-e]indazole-3-carboxylic acid.

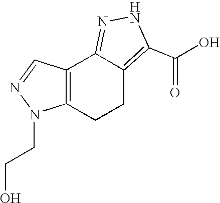

6-(2-hydroxyethyl)-2,4,5,6-tetrahydropyrazolo[3,4-e]indazole-3-carboxylic acid was prepared according to the procedure of 7-hydroxy-4,5-dihydro-2H-benzo[g]indazole-3-carboxylic acid to give the desired product as a white solid (83% yield). FABHRMS m/z 249.0988 (M+H, C₁₁H₁₃N₄O₃ requires 249.0988).

Anal. Calcd for C₁₁H₁₂N₄O₃: C, 53.22; H, 4.87; N, 22.57. Found: C, 52.86; H, 4.83; N, 22.49.

### EXAMPLE 372

This example illustrates the production of ethyl 1-[4-(aminosulfonyl)-phenyl]-6-benzyl-1,4,5,6-tetrahydropyrazolo[3,4-e]indazole-3-carboxylate.

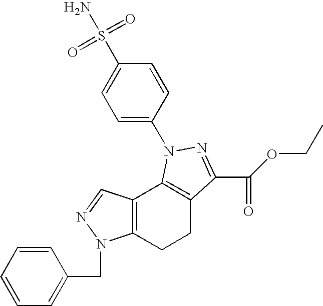

Ethyl 1-[4-(aminosulfonyl)phenyl]-6-benzyl-1,4,5,6-tetrahydropyrazolo[3,4-e]indazole-3-carboxylate- or ethyl 1-[4-(aminosulfonyl)phenyl]-6-benzyl-1,4,5,6-tetrahydropyrazolo[3,4-e]indazole-3-carboxylate was prepared according to the procedures for the production of ethyl (7-hydroxy-1-oxo-1,2,3,4-tetrahydronaphthalen-2-yl)(oxo)acetate and ethyl 1-[4-(aminosulfonyl)phenyl]-8-hydroxy-4,5-dihydro-1H-benzo[g]indazole-3-carboxylate to give the desired as a light tan solid (61% yield). FABHRMS m/z 478.1549 (M+H, C₂₄H₂₄N₅O₄S requires 478.1551). ¹H NMR (DMSO-d₆+5%TFA/300 MHz): 7.98 (AB quartet, 4H); 7.38-7.20 (m, 3H); 7.20-7.10 (m, 2H); 5.38 (s, 2H); 4.25 (q, 2H); 3.20-2.90 (m, 4H); 1.25 (t, 3H).

Anal. Calcd. for C₂₄H₂₃N₅O₄S: C, 60.36; H, 4.85; N, 14.67. Found: C, 60.60; H, 4.86; N, 14.71.

### EXAMPLE 373

This example illustrates the production of ethyl 1-[4-(aminosulfonyl)-phenyl]-1,4,5,6-tetrahydropyrazolo[3,4-e]indazole-3-carboxylate.

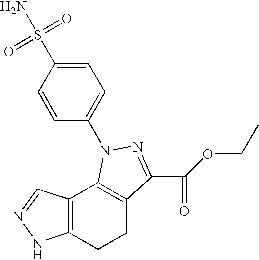

Ethyl 1-[4-(aminosulfonyl)phenyl]-6-benzyl-1,4,5,6-tetrahydropyrazolo[3,4-e]indazole-3-carboxylate- or ethyl 1-[4-(aminosulfonyl)phenyl]-6-benzyl-1,4,5,6-tetrahydropyrazolo[3,4-e]indazole-3-carboxylate (4.2 g, 0.009 mol), Pearleman's catalyst (1.5 g), glacial acetic acid (20 mL) and DMF (40 mL) were shaken at 55 psi H₂ for 72 hours. Contents were filtered through clay and the filtrate was concentrated in vacuo leaving a grey solid. The grey solid was triturated with acetonitrile to give an off-white solid, 3.0 g (86% yield). FABHRMS m/z 388.1080 (M+H, C₁₇H₁₈N₅O₄S requires 388.1124). ¹H NMR (DMSO-d₆/300 MHz): 7.96 (AB quartet, 4H); 7.55 (br s, 2H); 7.35 (br s, 1H); 4.35 (q, 2H); 3.30 (s, 2H); 3.15-3.05 (m, 2H); 3.00-2.85 (m, 2H); 1.35 (t, 3H).

Anal. Calcd for C₁₇H₁₇N₅O₄S: C, 52.70; H, 4.42; N, 18.08. Found: C, 52.47; H, 4.18; N, 17.89.

### EXAMPLE 374

This example illustrates the production of 1-[4-(aminosulfonyl)phenyl]-1,4,5,6-tetrahydropyrazolo[3,4-e]indazole-3-carboxamide.

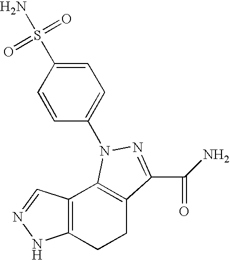

Ethyl 1-[4-(aminosulfonyl)phenyl]-1,4,5,6-tetrahydropyrazolo[3,4-e]indazole-3-carboxylate- or ethyl 1-[4-(aminosulfonyl)phenyl]-1,4,5,6-tetrahydropyrazolo[3,4-e]indazole-3-carboxylate was reacted according to the procedure for the production of 8-hydroxy-4,5-dihydro-2H-benzo[g]indazole-3-carboxamide to give the desired product as a white solid (86% yield). FABHRMS m/z 359.0939 (M+H, C₁₅H₁₅N₆O₃S requires 359.0926). ¹H NMR (DMSO-d₆/300 MHz): 7.99 (AB quartet, 4H); 7.58 (s, 1H); 7.50 (s, 1H); 7.35 (s, 1H); 3.35 (s, 2H); 3.10-3.00 (m, 2H); 2.90-2.80 (m, 2H).

Anal. Calcd for C₁₅H₁₄N₆O₃S: C, 50.27; H, 3.94; N, 23.45. Found: C, 50.07; H, 3.73; N, 23.08.

### EXAMPLE 375

This example illustrates the production of 1-[4-(aminosulfonyl)phenyl]-1,4,5,6-tetrahydropyrazolo[3,4-e]indazole-3-carboxylic acid.

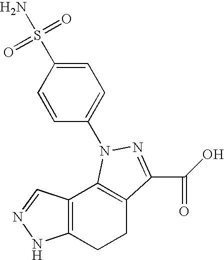

Ethyl 1-[4-(aminosulfonyl)phenyl]-1,4,5,6-tetrahydropyrazolo[3,4-e]indazole-3-carboxylate- or ethyl 1-[4-(aminosulfonyl)phenyl]-1,4,5,6-tetrahydropyrazolo[3,4-e]indazole-3-carboxylate was reacted according to the procedure of 7-hydroxy-4,5-dihydro-2H-benzo[g]indazole-3-carboxylic acid to give the desired product as a white solid (85% yield). FABHRMS m/z 360.0762 (M+H, C₁₅H₁₄N₅O₄S requires 360.0767).

Anal. Calcd for C₁₅H₁₃N₅O₄S: C, 46.03; H, 3.35; N, 17.89. Found: C, 46.67; H, 3.64; N, 17.93.

### EXAMPLE 376

This example illustrates the production of ethyl 6-benzyl-1-phenyl-1,4,5,6-tetrahydropyrazolo[3,4-e]indazole-3-carboxylate.

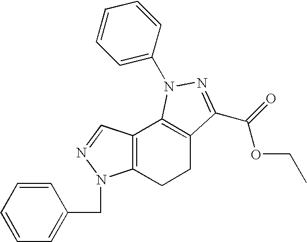

To N,N-dimethylacetamide dimethylacetal (250 mL) was added 1,3-cyclohexanedione (100 g, 0.89 mol). Contents were refluxed 2 hours, allowed to cool and concentrated in vacuo leaving an amber solid. After recrystallizing twice from EtOAc, the desired eneamine intermediate was obtained as light amber crystals, 64.2 g. The eneamine (21.7 g, 0.13 mol) and benzylhydrazine hydrochloride (25.5 g, 0.13 mol) were mixed in glacial acetic acid (200 mL) and stirred overnight. Contents were diluted with water, extracted with EtOAc, dried over MgSO₄, and concentrated in vacuo leaving the desired tetralone intermediate as an amber oil, 37.2 g. The tetralone was reacted with diethyl oxalate according to the procedure for the production of ethyl (7-hydroxy-1-oxo-1,2,3,4-tetrahydronaphthalen-2-yl)(oxo)acetate, to give the desired diketoester intermediate as an amber oil. The diketoester was reacted with phenylhydrazine according to the procedure for the production of ethyl 8-hydroxy-4,5-dihydro-2H-benzo[g]indazole-3-carboxylate, to give the desired product as white crystals (14% yield). FABHRMS m/z 399.1826 (M+H, C₂₄H₂₃N₄O₂ requires 399.1821).

Anal. Calcd for C₂₄H₂₂N₄O₂: C, 72.34; H, 5.57; N, 14.06. Found: C, 72.14; H, 5.41; N, 13.98.

### EXAMPLE 377

This example illustrates the production of ethyl 1-phenyl-1,4,5,6-tetrahydro-pyrazolo[3,4-e]indazole-3-carboxylate.

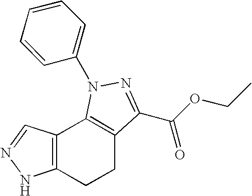

Ethyl 6-benzyl-1-phenyl-1,4,5,6-tetrahydropyrazolo[3,4-e]indazole-3-carboxylate (3.4 g, 0.009 mol) and Pearleman's catalyst (1.0 g) in ethanol (100 mL) were shaken at 55 psi H₂ and 55° C. for 72 hours. Contents were allowed to cool and filtered through clay. The filtrate was concentrated in vacuo leaving a white solid. Triteration of the solid with methanol gave the desired as a white solid, 1.0 g (36% yield). FABHRMS m/z 309.1343 (M+H, C₁₇H₁₇N₄O₂ requires 309.1352).

Anal. Calcd for C₁₇H₁₆N₄O₂: C, 66.22; H, 5.23; N, 18.17. Found: C, 66.06; H, 5.41; N, 17.58.

### EXAMPLE 378

This example illustrates the production of 1-phenyl-1,4,5,6-tetrahydro-pyrazolo[3,4-e]indazole-3-carboxamide.

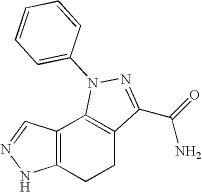

1-phenyl-1,4,5,6-tetrahydropyrazolo[3,4-e]indazole-3-carboxamide was prepared according to the procedure for the production of 8-hydroxy-4,5-dihydro-2H-benzo[g]indazole-3-carboxamide, at 100° C., to give the desired product as a grey solid (72% yield). FABHRMS m/z 280.1208 (M+H, C₁₅H₁₄N₅O requires 280.1198).

Anal. Calcd for C₁₅H₁₃N₅O (0.5H₂O): C, 62.49; H, 4.89; N, 24.29. Found: C, 62.94; H, 4.83; N, 23.76.

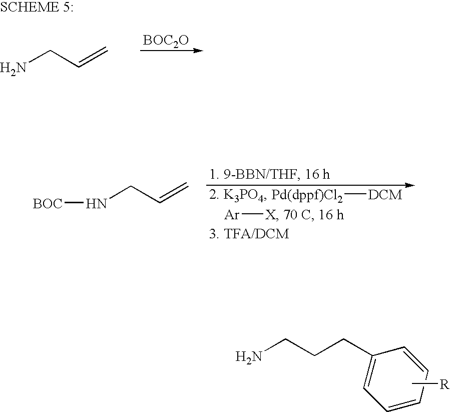

### EXAMPLE 379

This example illustrates the production of Boc-allyl amine. Boc-anhydride (1M THF, 70 mmol, 70 mL) was added to allyl amine (70 mmol, 4.0 g, 5.26 mL) slowly with stirring. The reaction solution stirred at 23° C. for 16 hours. The THF was removed in vacuo and the residue was reconstituted in dichloromethane (20 mL) and washed with water and brine (1×25 mL). The organic layer was dried (MgSO₄) and concentrated in vacuo. The product was isolated as a white low melting solid (10.25 g, 99% yield): ¹HNMR (400 MHz, CDCl₃) δ 5.853-5.770 (m, 1H), 5.278-5.069 (qm, 2H), 3.723 (bs, 2H), 1.429 (s, 9H).

### EXAMPLE 380

This example illustrates the production of 3-(4-tert-butylphenyl)-propylamine.

Tert-butyl allylcarbamate (7.0 mmol, 1.10 g) was weighed into a 100 mL flask with stir bar and 9-BBN solution (0.5M, THF, 7.7 mmol, 15.4 mL) was syringed carefully into the neat amine. The reaction stirred at 23° C. for 16 hours. The potassium phosphate (2M, 10.5 mmol, 5.25 mL) was added to the reaction mixture and stirred at 23° C. for 10 minutes. [1,1′-Bis(diphenylphosphino)ferrocene]dichloropalladium(II)/DCM complex (0.21 mmol, 171 mg) and the 4-t-butyl-phenylbromide (7.0 mmol, 1.49 g, 1.21 mL) were added to the basic reaction mixture and the slurry was heated to relfux for 16 hours. The crude reaction mixture was purified using a 20 g flash silica plug (eluting with 100% hexane to 50% ethylacetate/hexane). The isolated pale brown oil (2.96 g) was dissolved in 25%TFA/DCM (15 mL) and stirred for 30 minutes at 23° C. The reaction mixture was concentrated in vacuo, reconstituted in dichloromethane (20 mL) and washed with saturated bicarbonate solution (3×30 mL). The organic layer was concentrated in vacuo. The crude product was purified using a 20 g flash silica plug (eluting with 100% hexane to 100% ethyl acetate to 100% methanol). Isolated the product as a light tan solid (1.25 g, 94% yield): ¹HNMR (400 MHz, DMSO) δ 7.911 (bs, 2H), 7.200 (q, (a,b), 4H), 2.762 (m, 2H), 2.579 (t, 2H), 1.811 (m, 2H), 1.240 (s, 9H): m/z 192 (M+H).

### EXAMPLE 381

This example illustrates the production of 3-(4-chlorophenyl)propylamine.

The product was isolated as a tan semi-solid (231 mg, 45% yield): ¹HNMR (400 MHz, DMSO) δ 7.300 (q, a,b, 4H), 2.631 (m, 4H), 1.724 (m, 2H): m/z 170 (M+H).

### EXAMPLE 382

This example illustrates the production of 3-(4-bromophenyl)propylamine.

The product was isolated as a tan semi-solid (310 mg, 48% yield): ¹HNMR (400 MHz, DMSO) δ 7.380 (q, a,b, 4H), 2.639 (q, 4H), 1.724 (m, 2H): m/z 216 (M+H).

### EXAMPLE 383

This example illustrates the production of 3-(3-chlorophenyl)propylamine.

The product was isolated as a tan semi-solid (244 mg, 48% yield): ¹HNMR (400 MHz, DMSO) δ 7.323 (m, 3H), 7.220 (d, 1H), 2.664 (m, 4H), 1.796 (t, 2H): m/z 170 (M+H).

### EXAMPLE 384

This example illustrates the production of 3-[4-(methylsulfonyl) phenyl]propylamine. The product was isolated as a tan semi-solid (188 mg, 29% yield): ¹HNMR (400 MHz, DMSO) δ 7.610 (q, a,b, 4H), 3.220 (s, 3H), 2.783 (m, 4H), 1.873 (m, 2H): m/z 214 (M+H).

### EXAMPLE 385

This example illustrates the production of 3-(1,1′-biphenyl-4-yl)propylamine.

The product was isolated as a tan semi-solid (485 mg, 76% yield): ¹HNMR (400 MHz, DMSO) δ 7.649(m, 4H), 7.483 (t, 2H), 7.374 (m, 3H), 2.842 (t, 2H), 2.686 (t, 2H), 1.894 (t, 2H): m/z 212 (M+H).

The following compounds were synthesized in the same manner as 2-(3-{[2-(2-chlorophenyl)ethyl]amino}propyl)-4,5-dihydro-2H-pyrazolo[3,4-f]isoquinoline-3-carboxylic acid trifluoroacetate (cyclic pyrazole Bromide alkylation of amines) using the previously described amines.

### EXAMPLE 386

This example illustrates the production of 2-(3-{[3-(4-tert-butylphenyl)propyl]amino}propyl)-4,5-dihydro-2H-pyrazolo[3,4-f]isoquinoline-3-carboxylic acid trifluoroacetate.

The product was isolated as a white amorphous solid (148 mg, 30% yield):

¹HNMR (400 MHz, DMSO) δ 8.685 (s, 1H), 8.608 (d, 1H), 7.840 (d, 1H), 7.284 (d, 2H), 7.094 (d, 2H), 4.627 (t, 2H), 3.010 (s, 4H), 2.931 (m, 2H), 2.860 (m 2H0, 2.551 (t, 2H), 2.126 (m, 2H), 1.807 (m, 2H), 1.222 (s, 9H): m/z 447 (M+H).

### EXAMPLE 387

This example illustrates the production of 2-(3-{[3-(4-chlorophenyl)propyl]amino}propyl)-4,5-dihydro-2H-pyrazolo[3,4-f]isoquinoline-3-carboxylic acid trifluoroacetate.

The product was isolated as a white amorphous solid (84 mg, 18% yield):

¹HNMR (400 MHz, DMSO) δ 8.726 (bs, 1H), 8.644 (d, 1H), 7.895 (d, 1H), 7.334 (d, 2H), 7.211 (d, 2H), 4.632 (t, 2H), 3.025 (s, 4H), 2.931 (m, 2H), 2.860 (m, 2H), 2.593 (t, 2H), 2.126 (m, 2H), 1.807 (m, 2H): m/z 425 (M+H).

### EXAMPLE 388

This example illustrates the production of 2-(3-{[3-(4-bromophenyl)propyl]amino}propyl)-4,5-dihydro-2H-pyrazolo[3,4-f]isoquinoline-3-carboxylic acid trifluoroacetate.

The product was isolated as a white amorphous solid (71 mg, 14% yield):

¹HNMR (400 MHz, DMSO) δ 8.726 (bs, 1H), 8.644 (d, 1H), 7.895 (d, 1H), 7.449 (d, 2H), 7.154 (d, 2H), 4.632 (t, 2H), 3.006 (s, 4H), 2.931 (m, 2H), 2.860 (m, 2H), 2.593 (t, 2H), 2.126 (m, 2H), 1.807 (m, 2H): m/z 471 (M+H).

### EXAMPLE 389

This example illustrates the production of 2-(3-{[3-(3-chlorophenyl)propyl]amino}propyl)-4,5-dihydro-2H-pyrazolo[3,4-f]isoquinoline-3-carboxylic acid trifluoroacetate.

The product was isolated as a white amorphous solid (58 mg, 12% yield):

¹HNMR (400 MHz, DMSO) δ 8.727 (s, 1H), 8.644 (d, 1H), 7.899 (d, 1H), 7.268 (m, 3H), 7.156 (d, 1H), 4.634 (t, 2H), 3.027 (s, 4H), 2.931 (m, 2H), 2.862 (m, 2H), 2.613 (t, 2H), 2.153 (m, 2H), 1.830 (m, 2H): m/z 425 (M+H).

### EXAMPLE 390

This example illustrates the production of 2-[3-({3-[4-(methylsulfonyl)phenyl]propyl}amino)propyl]-4,5-dihydro-2H-pyrazolo[3,4-f]isoquinoline-3-carboxylic acid trifluoroacetate.

The product was isolated as a white amorphous solid (52 mg, 12% yield):

¹HNMR (400 MHz, DMSO) δ 8.662 (bs, 1H), 8.591 (d, 1H), 7.841 (d, 2H), 7.806 (d, 1H), 7.466 (d, 2H), 4.626 (t, 2H), 3.460 (dq, 4H), 3.159 (s, 3H), 2.931 (m, 2H), 2.860 (m, 2H), 2.718 (t, 2H), 2.126 (m, 2H), 1.857 (m, 2H): m/z 469 (M+H).

### EXAMPLE 391

This example illustrates the production of 2-(3-{[3-(1,1′-biphenyl-4-yl)propyl]amino}propyl)-4,5-dihydro-2H-pyrazolo[3,4-f]isoquinoline-3-carboxylic acid trifluoroacetate.

The product was isolated as a white amorphous solid (52 mg, 12% yield):

¹HNMR (400 MHz, DMSO) δ 8.684 (s, 1H), 8.600 (d, 1H), 7.839 (d, 1H), 7.613-7.555 (m, 4H), 7.422 (t, 2H), 7.320 (t, 1H), 7.277 (d, 2H), 4.634 (t, 2H), 3.008 (s, 4H), 2.940 (m, 4H), 2.645 (t, 2H), 2.140 (m, 2H), 1.865 (m, 2H): m/z 467 (M+H).

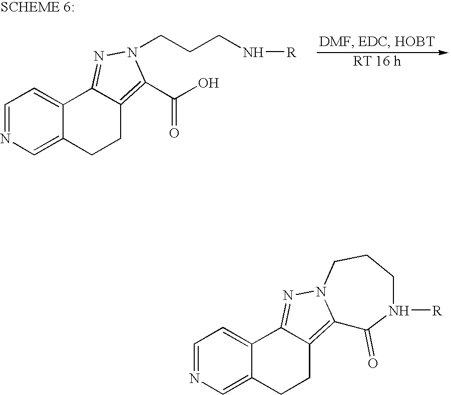

### EXAMPLE 392

This example illustrates the production of 8-[2-(2-chlorophenyl)ethyl]-5,6,8,9,10,11-hexahydro-7H-[1,4]diazepino[1′,2′:1,5]pyrazolo[3,4-f]isoquinolin-7-one trifluoroacetate.

2-(3-{[2-(2-chlorophenyl)ethyl]amino}propyl)-4,5-dihydro-2H-pyrazolo[3,4-f]isoquinoline-3-carboxylic acid trifluoroacetate (0.38 mmol, 200 mg) was dissolved in DMF (5.0 mL) and EDC-HCl (0.57 mmol, 109 mg), and HOBT (0.46 mmol, 62 mg) were added. The reaction mixture was stirred at 23° C. for 16 hours. The reaction mixture was diluted with ethyl acetate (20 mL) and washed with water (2×25 mL). The organic layer was concentrated in vacuo, reconstituted in acetonitrile and purified using reverse phase chromatography. The product was isolated as a white amorphous solid (36.0 mg, 24% yield).

¹HNMR (400 MHz, DMSO) δ 8.805 (s, 1H), 8.726 (d, 1H), 8.045 (d, 1H), 7.434 (t, 2H), 7.319 (m, 2H), 4.406 (t, 2H), 3.789 (t, 2H), 3.378 (t, 2H), 3.089 (t, 4H), 2.917 (t, 2H), 2.149 (t, 2H): m/z 394 (M+H).

A general method for the synthesis of pyrazoles described in the following examples are provided below.

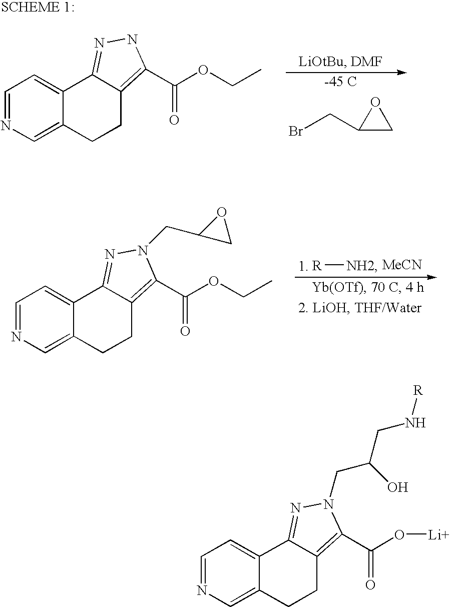

### EXAMPLE 393

This example illustrates the production of ethyl 2-(oxiran-2-ylmethyl)-4,5-dihydro-2H-pyrazolo[3,4-f]isoquinoline-3-carboxylate.

The pyrazole ester (1.23 mmol, 300 mg) was dissolved in DMF (10 mL) and cooled to −45° C. in a dry ice/acetonitrile bath. Lithium tert-butoxide (1M THF, 1.85 mmol, 1.85 mL) was added slowly being sure to maintain −45° C. The cold solution stirred for 1.5 h. Epibromohydrin (1.85 mmol, 0.253 g, 159 μL) was added via syringe to the cold DMF solution and the ice bath was removed. The reaction was allowed to warm to 23° C. over 16 h. The crude reaction was diluted with ethyl acetate (20 mL) and washed with water (1×10 mL) and brine (1×20 mL). The organic layer was dried (MgSO₄) and concentrated in vacuo. The crude oil was purified via gravity feed chromatography using 60% ethyl acetate/hexane. The product was isolated as a clear oil (70 mg, 19% yield):

¹HNMR (400 MHz, CDCl₃) δ 8.602 (bs, 2H), 7.811(d, 1H), 4.825 (qd, 2H), 4.397 (q, 2H), 3.411 (bs, 1H), 3.035 (qd, 4H), 2.833 (t, 1H), 2.633 (m, 1H), 1.432 (t, 3H): m/z 300 (M+H).

### EXAMPLE 394

This example illustrates the production of 9-(morpholin-4-5,6,9,10-tetrahydro-7H-[1,4]oxazino[4′,3′:1,5]pyrazolo[3,4-f]isoquinolin-7-one.

Ethyl 2-(oxiran-2-ylmethyl)-4,5-dihydro-2H-pyrazolo[3,4-f]isoquinoline-3-carboxylate (1.34 mmol, 400 mg) was dissolved in acetonitrile (4 mL) and the morpholine (2.0 mmol, 175 mg, 175 μL) and the ytterbium triflate (0.2 mmol, 124 mg) were added. The vial was capped and heated to 70° C. for 4 h with stirring. After four hours of heating, the reaction vessel was allowed to cool and upon cooling the desired product precipitated and was collected via filtration. The product was isolated as a white solid (55 mg, 12% yield):

¹HNMR (400 MHz, CDCl₃) δ 8.538 (bs, 2H), 7.662 (d, 1H), 4.916 (m, 1H), 4.501(qd, 2H), 3.708 (bs, 4H), 3.178-3.079 (m, 2H), 2.995 (d, 2H), 2.872-2.803 (m, 2H), 2.606 (bs, 4H): m/z 341 (M+H). The following lactones were made (using the appropriate amine) in the same manner as 9-(morpholin-4-ylmethyl)-5,6,9,10-tetrahydro-7H-[1,4]oxazino[4′,3′:1,5]pyrazolo[3,4-f]isoquinolin-7-one:

### EXAMPLE 395

This example illustrates the production of 9-{[(2-thien-2-ylethyl)amino]methyl}-5,6,9,10-tetrahydro-7H-[1,4]oxazino[4′,3′:1,5]pyrazolo[3,4-f]isoquinolin-7-one trifluoroacetate.

The product was isolated as a pale yellow solid (129 mg, 23% yield): ¹HNMR (400 MHz, DMSO) δ 8.750 (s 1H), 8.655(d, 1H), 7.985 (d, 1H), 7.337 (d, 1H), 6.936 (m, 2H), 4.358 (dd, 2H), 4.220 (dd, 2H), 3.82 (m, 1H), 3.582 (m, 1H), 3.460 (dd, 1H), 3.124 (t, 2H), 3.041 (m, 4H), 2.901 (t, 2H),: m/z 381 (M+H).

### EXAMPLE 396

This example illustrates the production of 9-{[(4-aminocyclohexyl)amino]methyl}-5,6,9,10-tetrahydro-7H-[1,4]oxazino-[4′,3′:1,5]pyrazolo[3,4-f]isoquinolin-7-one trifluoroacetate.

The product was isolated as a tan solid (338 mg, 61% yield): ¹HNMR (400 MHz, DMSO) δ 8.682 (s, 1H), 8.612 (d, 1H), 7.880(d, 1H), 4.502 (dd, 2H), 3.454 (t, 2H), 3.381 (t, 2H), 2.979 (t, 2H), 1.941 (d, 4H), 1.326 (t, 4H): m/z 368 (M+H).

### EXAMPLE 397

This example illustrates the production of 9-(aminomethyl)-5,6,9,10-tetrahydro-7H-[1,4]oxazino[4′,3′:1,5]pyrazolo[3,4-f]isoquinolin-7-one trifluoroacetate.

The product was isolated as a yellow solid (85.5 mg, 21% yield): ¹HNMR (400 MHz, DMSO) δ 8.726 (s, 1H), 8.651 (d, 1H), 7.968 (d, 1H), 4.588 (dd, 2H), 4.282-4.244 (3H), 3.430 (dd, 4H): m/z 271 (M+H).

### EXAMPLE 398

This example illustrates the production of lithium 2-(2-hydroxy-3-morpholin-4-ylpropyl)-4,5-dihydro-2H-pyrazolo[3,4-f]isoquinoline-3-carboxylate.

9-(morpholin-4-ylmethyl)-5,6,9,10-tetrahydro-7H-[1,4]oxazino-[4′,3′:1,5]pyrazolo[3,4-f]isoquinolin-7-one (0.41 mmol, 139.5 mg) was dissolved in 50% THF/water (4.0 mL) and lithium hydroxide (2M, 0.21 mL) was added. The reaction mixture stirred at 23° C. for 3hours. The sample was concentrated under nitrogen stream. The residue was triturated with acetonitrile and sonicated to produce a fine powder. The product was collected via filtration and isolated as a light tan solid (140.4 mg, 95% yield):

¹HNMR (400 MHz, DMSO) δ 8.468 (d, 2H), 7.655 (s, 1H), 7.544 (s, 1H), 4.601 (bs, 2H), 3.947 (m, 1H), 3.633 (s, 4H), 2.931-2.88 (d, 4H), 2.555 (m, 6H): m/z 359 (M+H). The following compounds were hydrolyzed from the lactone to the acid in the same manner as lithium 2-(2-hydroxy-3-morpholin-4-ylpropyl)-4,5-dihydro-2H-pyrazolo[3,4-f]isoquinoline-3-carboxylate

### EXAMPLE 399

This example illustrates that MK2 knock-out mice (MK2 (−/−)) are resistant to the formation of K/BN serum-induced arthritis and that compounds that inhibit MK-2 should be effective for the prevention and treatment of TNFα-mediated diseases or disorders.

A strain of mice has been reported that develops symptoms similar to human rheumatoid arthritis. The mice were designated K/B×N mice. See, Wipke, B. T. and P. M. Allen, J. of Immunology, 167:1601-1608 (2001). Serum from the mice can be injected into host animals to provoke a typical RA response. The progression of the RA symptoms in the mice is measured by measuring paw thickness as a function of time.

In the present example, host mice having normal MK-2 production (MK2 (+/+)) were genetically altered by disabling the gene encoding MK-2 to produce mice having no capability of endogenous synthesis of active MK-2 (MK2 (−/−)). Normal host mice (MK2 (+/+)) and MK-2 knock-out mice (MK2 (−/−), were separated into four groups with each group containing both male and female mice. All groups of mice were treated similarly, except that one group (Normal), Composed of MK2 (+/+) mice that served as the control group, was not injected with serum from K/B×N mice, while the other three groups were injected with K/B×N serum at day 0. The other three groups of mice were MK2 (+/+), MK2 (−/−), and Anti-TNF. The Anti-TNF group was composed of MK2 (+/+) mice which were also injected at day) with anti-TNF antibody. The paw thickness of all mice was measured immediately after the injections on day 0, and then on each successive day thereafter for 7 days.

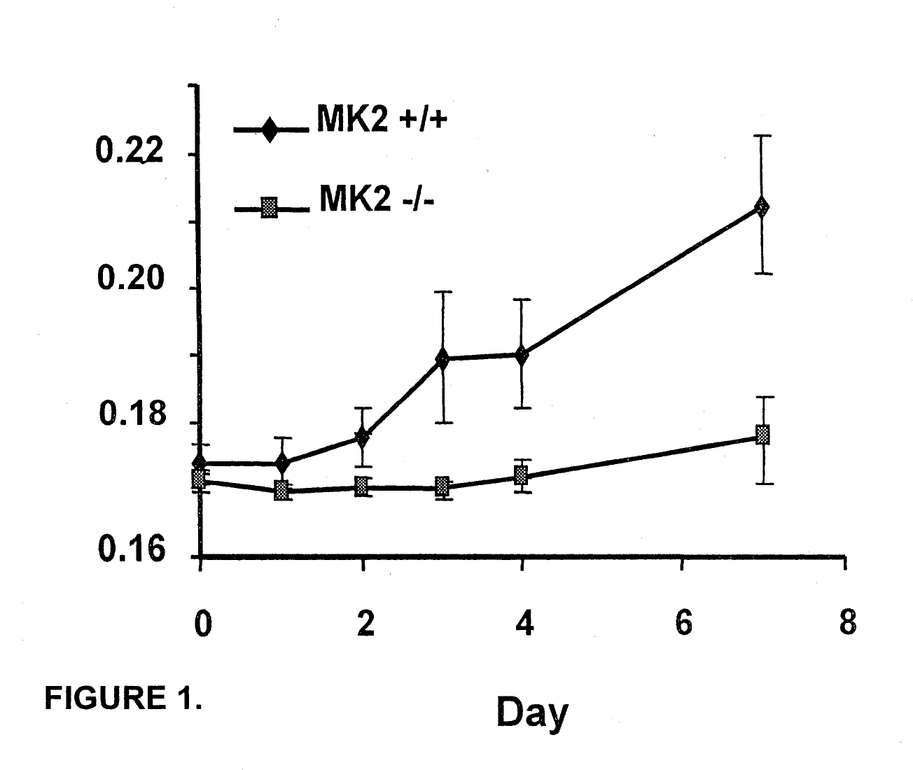

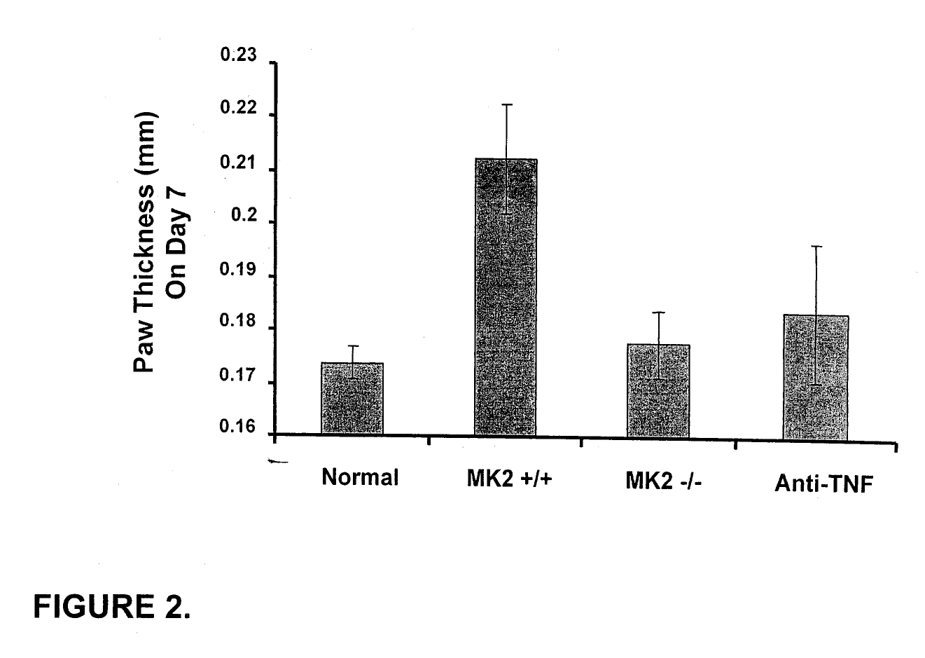

FIG. 1 is a graph that shows paw thickness as a function of time from day 0 to day 7 for MK2 (+/+) and MK2 (−/−) mice, which have received serum injection. It can be seen that paw thickness increased significantly for MK2(+/+) mice, whereas there was substantially no increase in paw thickness for MK2 knock-out mice. This indicated the requirement for a functioning MK2 regulatory system to the inflammatory response caused by the serum challenge. When anti-TNF antibody was administered to the MK2 (+/+) mice along with the serum injection, the swelling response was significantly reduced. This can be seen in FIG. 2, which is a bar chart showing paw thickness at seven days after injection for normal mice, MK2 (+/+) mice receiving serum, MK2 (−/−) mice receiving serum, and MK2 (+/+) mice receiving serum and anti-TNF antibody.

This data shows that the MK-2 knock-out mice show no arthritic response to a serum challenge, whereas MK2 (+/+) mice show a normal response. Treatment of MK2 (+/+) mice that receive a serum challenge with anti-TNF antibody reduces the response back to near-normal levels. This illustrates the utility of the MK-2 regulatory system as a potential control point for the modulation of TNFα production, and indicates that such regulation could serve as a treatment for inflammation—such as that caused by arthritis, for example. It further shows that MK-2 inhibition can have a beneficial effect on inflammation, and indicates that administration of an MK-2 inhibitor can be an effective method of preventing or treating TNFα modulated diseases or disorders.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the-several advantages of the invention are achieved and other advantageous results obtained.

As various changes could be made in the above methods and compositions without departing from the scope of the invention, it is intended that all matter contained in the above description shall be interpreted as illustrative and not in a limiting sense.

What is claimed is:

1. A cyclic pyrazole MK-2 inhibiting compound having the structure: 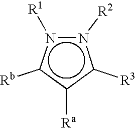
where:
R^{a} and R^{b} join to form a ring structure selected from the group consisting of: 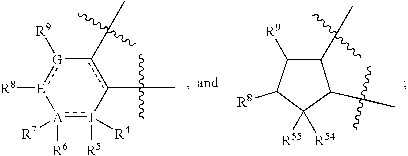
A, E, G and J are carbon, or optionally, one of A, E, G and J is selected from nitrogen, sulfur, or oxygen;
when A, E, G, or J is oxygen or sulfur, no substituent groups are bonded to the oxygen or the sulfur;
when A, E, G, or J is nitrogen, the nitrogen is optionally unsubstituted, or is substituted with R⁹;
each of R¹, R², R⁴ and R⁶ is optionally absent, or is independently selected from the R³ groups;
R³, R⁵, R⁷, R⁸, R⁹, R⁴⁰, R⁵⁴ and R⁵⁵ are each independently selected from —H, C₁-C₆ alkyl, C₂-C₆ alkenyl, C₂-C₆ alkynyl, nitro C₁-C₆ alkyl, azido C₁-C₆ alkyl, amino, NHR¹⁰, NR¹¹R¹², C₁-C₆ alkyl-NHR¹⁰, C₁-C₆-alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁵;
R¹⁰, R¹¹, are each independently selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, CO₂R¹⁹, CR²⁰, CONHR¹⁹, CON(R¹⁹)₂, SR¹⁸, SOR²⁰, SO₂R²⁰, C₁-C₆alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, arylaryl, arylheteroaryl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹², R¹³ are each independently selected from —H, C₁-C₆ alkyl, C₂-C₆ alkenyl, C₂-C₆ alkynyl, C₁-C₆ alkyl-NHR¹⁶, C₁-C₆ alkyl-NR¹⁶R¹⁷, C₁-C₄ alkyl-OR¹⁸, aryl, heteroaryl, heterocyclyl, CO₂R¹⁹, COR²⁰, CONHR¹⁹, CON(R¹⁹)₂, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆-alkyl-SOR²⁹, C₁-C₆ alkyl-SO₂R²⁰, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁴ is selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, SR¹⁸, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁵ is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁶ and R¹⁷ are each independently selected from —H, C₁-C₆ alkyl, C₂-C₆ alkenyl, C₂-C₆ alkynyl, amino, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁶, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R¹⁸, R¹⁹ are each independently selected from —H, C₁-C₆ alkyl, C₂-C₆ alkenyl, C₂-C₆ alkynyl, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁷, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²⁰ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, O—R²⁴, C₁-C₄ alkyl-OR²⁴, aryl, heteroaryl, heterocyclyl, SR²⁴, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, arylalkoxyalkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²¹ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, nitro, cyano, O—R²⁴, C₁-C₄ alkyl-OR²⁴, aryl, heteroaryl, heterocyclyl, COR²⁶, SR²⁴, SOR²⁶, SO₂R²⁶, C₁-C₆ alkyl-COR²⁶, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²² and R²³ are each independently selected from —H, C₁-C₆ alkyl, C₂-C₆ alkenyl, C₂-C₆ alkynyl, amino, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁴ and R²⁵ are each independently selected from —H, C₁-C₆ alkyl, C₂-C₆ alkenyl, C₂-C₆ alkynyl, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁶ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, O—R³⁰, C₁-C₄ alkyl-OR³⁰, aryl, heteroaryl, heterocyclyl, SR³⁰, C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁷ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, nitro, cyano, O—R³⁰, C₁-C₄ alkyl-OR³⁰, aryl, heteroaryl, heterocyclyl, COR³², SR³⁰, SOR³², SO₂R³², C₁-C₆ alkyl-COR³², C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁸ and R²⁹ are each independently selected from —H, C₁-C₆ alkyl, C₂-C₆ alkenyl, C₂-C₆ alkynyl, amino, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁰ and R³¹ are each independently selected from —H, C₁-C₆ alkyl, C₂—, C₆ alkenyl, C₂-C₆ alkynyl, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³² is selected from —H, C₁-C₆ alkyl, C₂-C₆ alkenyl, C₂-C₆ alkynyl, amino, NHR³⁴, NR³⁴R³⁵, C₁-C₆ alkyl-NHR³⁴, C₁-C₆ alkyl-NR³⁴R³⁵, O—R³⁶, C₁-C₄ alkyl-OR³⁶, aryl, heteroaryl, heterocyclyl, SR³⁵, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³³ is selected from —H, C₁-C₆ alkyl, C₂-C₆ alkenyl, C₂-C₆ alkynyl, amino, NHR³⁴, NR³⁴R³⁵, C₁-C₆ alkyl-NHR³¹, C₁-C₆ alkyl-NR³⁴R³⁵, nitro, cyano, O—R³⁶, C₁-C₄ alkyl-OR³⁶, aryl, heteroaryl, heterocyclyl, COR³⁸, SR³⁵, SOR³⁸, SO₂R³⁸, C₁-C₆ alkyl-COR³⁸, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁴, R³⁵, R³⁶ and R³⁷ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁸ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁹ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, arylalkenyl, heterocyclylalkenyl, arylheterocyclylalkenyl, heterocyclylarylalkenyl, or heteroarylalkyl; and
R² and R³, or R⁸ and R⁹, optionally join to form a saturated or unsaturated ring of 5, 6, 7, or 8 atoms, where the atoms in the ring are independently selected from CR⁴⁰, C(R⁴⁰)₂, C═O, N, NR⁴⁰, O, S, C═S, S═O, or SO₂.

2. A cyclic pyrazole MK-2 inhibiting compound having the structure: 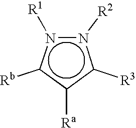
where:
R^{a} and R^{b} join to form a ring structure selected from the group consisting of: 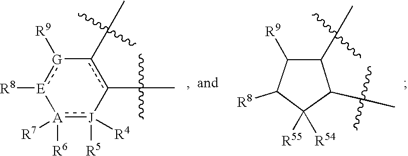
A, E, G and J are carbon, or optionally, one of A, E, G and J is selected from nitrogen, sulfur, or oxygen;
when A, E, G, or J is oxygen or sulfur, no substituent groups are bonded to the oxygen or the sulfur;
when A, E, G, or J is nitrogen, the nitrogen is optionally unsubstituted, or is substituted with R⁹;
each of R¹, R², R⁴ and R⁶ is optionally absent, or is independently selected from the R³ groups;
R³, R⁵, R⁷, R⁸, R⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴ and R⁵⁵ are each independently selected from —H, C₁-C₆ alkyl, C₂-C₆ alkenyl, C₂-C₆ alkynyl, nitro C₁-C₆ alkyl, azido C₁-C₆ alkyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁵, or R⁸ and R⁹ join to form a ring structure selected from: 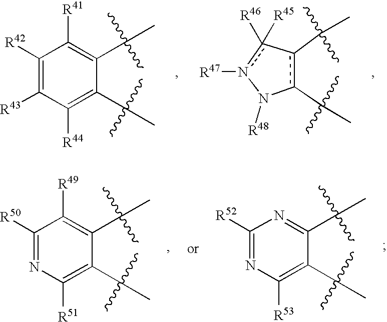
R¹⁰, R¹¹, are each independently selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, CO₂R¹⁹, COR²⁰, CONHR¹⁹, CON(R¹⁹)₂, SR¹⁸, SOR²⁰, SO₂R²⁰, C₁-C₆alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, arylaryl, arylheteroaryl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹², R¹³ are each independently selected from —H, C₁-C₆ alkyl, C₂-C₆ alkenyl, C₂-C₆ alkynyl, C₁-C₆ alkyl-NHR¹⁶, C₁-C₆ alkyl-NR¹⁶R¹⁷, C₁-C₄ alkyl-OR¹⁸, aryl, heteroaryl, heterocyclyl, CO₂R¹⁹, COR²⁰, CONHR¹⁹, CON(R¹⁹)₂, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁴ is selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, SR¹⁸, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁵ is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁶ and R¹⁷ are each independently selected from —H, C₁-C₆ alkyl, C₂-C₆ alkenyl, C₂-C₆ alkynyl, amino, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁶, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R¹⁸, R¹⁹ are each independently selected from —H, C₁-C₆ alkyl, C₂-C₆ alkenyl, C₂-C₆ alkynyl, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁷, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²⁰ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, O—R²⁴, C₁-C₄ alkyl-OR²⁴, aryl, heteroaryl, heterocyclyl, SR²⁴, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, arylalkoxyalkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²¹ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, nitro, cyano, O—R²⁴, C₁-C₄ alkyl-OR²⁴, aryl, heteroaryl, heterocyclyl, COR²⁶, SR²⁴, SOR²⁶, SO₂R²⁶, C₁-C₆ alkyl-COR²⁶, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²² and R²³ are each independently selected from —H, C₁-C₆ alkyl, C₂-C₆ alkenyl, C₂-C₆ alkynyl, amino, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁴ and R²⁵ are each independently selected from —H, C₁-C₆ alkyl, C₂-C₆ alkenyl, C₂-C₆ alkynyl, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁶ is selected from —H, C₁-C₆ alkyl, C₂-C₆—alkenyl, C₂-C₆ alkynyl, amino, NHR²⁸, NR²⁸R²⁹, C₁-C₆ alkyl-NHR²⁸, C₁-C₆ alkyl-NR²⁸R²⁹, O—R³⁰, C₁-C₄ alkyl-OR³⁰, aryl, heteroaryl, heterocyclyl, SR³⁰, C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-S R³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁷ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, nitro, cyano, O—R³⁰, C₁-C₄ alkyl-OR³⁰, aryl, heteroaryl, heterocyclyl, COR³², SR³⁰, SOR³², SO₂R³², C₁-C₆ alkyl-COR³², C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁸ and R²⁹ are each independently selected from —H, C₁-C₆ alkyl, C₂-C₆ alkenyl, C₂-C₆ alkynyl, amino, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups-defined by R³⁹;
R³⁰ and R³¹ are each independently selected from —H, C₁-C₆ alkyl, C₂-C₆ alkenyl, C₂-C₆ alkynyl, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³² is selected from —H, C₁-C₆ alkyl, C₂-C₆ alkenyl, C₂-C₆ alkynyl, amino, NHR³⁴, NR³⁴R³⁵, C₁-C₆ alkyl-NHR³⁴, C₁-C₆ alkyl-NR³⁴R³⁵, O—R³⁶, C₁-C₄ alkyl-OR³⁶, aryl, heteroaryl, heterocyclyl, SR³⁶, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³³ is selected from —H, C₁-C₆ alkyl, C₂-C₆ alkenyl, C₂-C₆ alkynyl, amino, NHR³⁴, NR³⁴R³⁵, C₁-C₆ alkyl-NHR³¹, C₁-C₆ alkyl-NR³⁴R³⁵, nitro, cyano, O—R³⁶, C₁-C₄ alkyl-OR³⁶, aryl, heteroaryl, heterocyclyl, COR³⁸, SR³⁵, SOR³⁸, SO₂R³⁸, C₁-C₆ alkyl-COR³⁸, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁴, R³⁵, R³⁶ and R³⁷ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁸ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁹ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, arylalkenyl, heterocyclylalkenyl, arylheterocyclylalkenyl, heterocyclylarylalkenyl, or heteroarylalkyl; and
R² and R³ optionally join to form a saturated or unsaturated ring of 5, 6, 7, or 8 atoms, where the atoms in the ring are independently selected from CR⁴⁰, C(R⁴⁰)₂, C═O, N, NR⁴⁰, O, S, C═S, S═O, or SO₂.

3. A cyclic pyrazole MK-2 inhibiting compound having the structure: 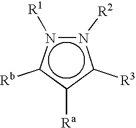
where:
R^{a} and R^{b} join to form a ring structure selected from the group consisting of: 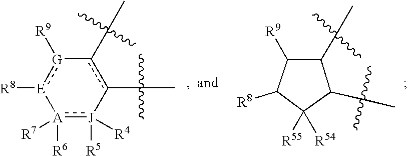
A, E, G and J are carbon, or optionally, one of A, E, G and J is selected from nitrogen, sulfur, or oxygen;
when A, E, G, or J is oxygen or sulfur, no substituent groups are bonded to the oxygen or the sulfur;
when A, E, G, or J is nitrogen, the nitrogen is optionally unsubstituted, or is substituted with R⁹;
R¹ is optionally absent, or is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, or C₇-C₁₀ mono- and bicyclic cycloalkyl, wherein mono- and bicyclic cycloalkyl are optionally substituted with one or more of the groups defined by R¹⁵;
each of R², R⁴ and R⁶ is optionally absent, or is independently selected from the R³ groups;
R³, R⁵, R⁷, R⁸, R⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴ and R⁵⁵ are each independently selected from —H, C₁-C₆ alkyl, C₂-C₆ alkenyl, C₂-C₆ alkynyl, nitro C₁-C₆ alkyl, azido C₁-C₆ alkyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁵; or R⁸ and R⁹ join to form a ring structure selected from: 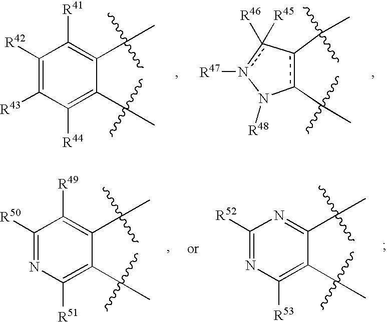
R¹⁰, R¹¹, are each independently selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, CO₂R¹⁹, CR²⁰, CONHR¹⁹, CON(R¹⁹)₂, SR¹⁸, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, arylaryl, arylheteroaryl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹², R¹³ are each independently selected from —H, C₁-C₆ alkyl, C₂-C₆ alkenyl, C₂-C₆ alkynyl, C₁-C₆ alkyl-NHR¹⁶, C₁-C₆ alkyl-NR¹⁶R¹⁷, C₁-C₄ alkyl-OR¹⁸, aryl, heteroaryl, heterocyclyl, CO₂R¹⁹, COR²⁰, CONHR¹⁹, CON(R¹⁹)₂, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁴ is selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, SR¹⁸, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁵ is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁶ and R¹⁷ are each independently selected from —H, C₁-C₆ alkyl, C₂-C₆ alkenyl, C₂-C₆ alkynyl, amino, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁶, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R¹⁸, R¹⁹ are each independently selected from —H, C₁-C₆ alkyl, C₂-C₆ alkenyl, C₂-C₆ alkynyl, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁷, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²⁰ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R?³, O—R²⁴, C₁-C₄ alkyl-OR²⁴, aryl, heteroaryl, heterocyclyl, SR²⁴, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, arylalkoxyalkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²¹ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, nitro, cyano, O—R²⁴, C₁-C₄ alkyl-OR²⁴, aryl, heteroaryl, heterocyclyl, COR²⁶, SR²⁴, SOR²⁶, SO₂R²⁶, C₁-C₆ alkyl-COR²⁶, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²² and R²³ are each independently selected from —H, C₁-C₆ alkyl, C₂-C₆ alkenyl, C₂-C₆ alkynyl, amino, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁴ and R²⁵ are each independently selected from —H, C₁-C₆ alkyl, C₂-C₆ alkenyl, C₂-C₆ alkynyl, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁶ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, O—R³⁰, C₁-C₄ alkyl-OR³⁰, aryl, heteroaryl, heterocyclyl, SR³⁰, C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁷ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, nitro, cyano, O—R³⁰, C₁-C₄ alkyl-OR³⁰, aryl, heteroaryl, heterocyclyl, COR³², SR³⁰, SOR³², SO₂R³², C₁-C₆ alkyl-COR³², C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁸ and R²⁹ are each independently selected from —H, C₁-C₆ alkyl, C₂-C₆ alkenyl, C₂-C₆ alkynyl, amino, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁵, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁵, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁰ and R³¹ are each independently selected from —H, C₁-C₆ alkyl, C₂-C₆ alkenyl, C₂-C₆ alkynyl, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³² is selected from —H, C₁-C₆ alkyl, C₂-C₆ alkenyl, C₂-C₆ alkynyl, amino, NHR³⁴, NR³⁴R³⁵, C₁-C₆ alkyl-NHR³⁴, C₁-C₆ alkyl-NR³⁴R³⁵, O—R³⁶, C₁-C₄ alkyl-OR³⁶, aryl, heteroaryl, heterocyclyl, SR³⁶, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³³ is selected from —H, C₁-C₆ alkyl, C₂-C₆ alkenyl, C₂-C₆ alkynyl, amino, NHR³⁴, NR³⁴R³⁵, C₁-C₆ alkyl-NHR³¹, C₁-C₆ alkyl-NR³⁴R³⁵, nitro, cyano, O—R³⁶, C₁-C₄ alkyl-OR³⁶, aryl, heteroaryl, heterocyclyl, COR³⁸, SR³⁵, SOR³⁸, SO₂R³⁸, C₁-C₆ alkyl-COR³⁸, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁴, R³⁵, R³⁶ and R³⁷ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁸ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁹ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, arylalkenyl, heterocyclylalkenyl, arylheterocyclylalkenyl, heterocyclylarylalkenyl, or heteroarylalkyl; and
R² and R³ optionally join to form a saturated or unsaturated ring of 5, 6, 7, or 8 atoms, where the atoms in the ring are independently selected from CR⁴⁰, C(R⁴⁰)₂, C═O, N, NR⁴⁰, O, S, C═S, S═O, or SO₂.

4. A cyclic pyrazole MK-2 inhibiting compound having the structure: 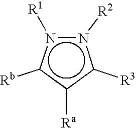
where:
R^{a} and R^{b} join to form a ring structure selected from the group consisting of: 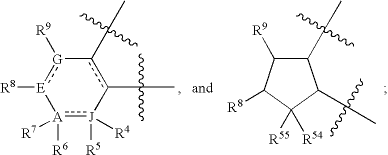
A, E, G and J are carbon, or optionally, one of A, E, G and J is selected from nitrogen, sulfur, or oxygen;
when A, E, G, or J is oxygen or sulfur, no substituent groups are bonded to the oxygen or the sulfur;
when A, E, G, or J is nitrogen, the nitrogen is optionally unsubstituted, or is substituted with R⁹;
each of R¹, R², R⁴ and R⁶ is optionally absent, or is independently selected from the R³ groups;
R³, R⁵, R⁷, R⁴⁰, R⁴¹, R⁴², R⁴³, R⁴⁴, R⁵⁴ and R⁵⁵ are each independently selected from —H, C₁-C₆ alkyl, C₂-C₆ alkenyl, C₂-C₆ alkynyl, nitro C₁-C₆ alkyl, azido C₁-C₆ alkyl, amino, NHR¹⁰, NR¹¹R¹², C₁-C₆ alkyl-NHR¹⁰, C₁-C₆ alkyl-NR¹¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁵;
R⁸ and R⁹ join to form the ring structure: 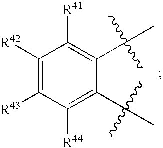
R¹⁰, R¹¹, are each independently selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C6 alkyl-NR¹⁶R¹⁷, O—R¹⁸, C₁-C₄ alkyl-OR¹⁸, aryl, heteroaryl, heterocyclyl, CO₂R¹⁹, COR²⁰, CONHR¹⁹, CON(R¹⁹)₂, SR¹⁸, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, arylaryl, arylheteroaryl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹², R¹³ are each independently selected from —H, C₁-C₆ alkyl, C₂-C₆ alkenyl, C₂-C₆ alkynyl, C₁-C₆ alkyl-NHR¹⁵, C₁-C₆ alkyl-NR¹⁶R¹⁷, C₁-C₄ alkyl-OR¹⁸, aryl, heteroaryl, heterocyclyl, CO₂R¹⁹, COR²⁰, CONHR¹⁹, CON(R¹⁹)₂, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR₁₃, C₁-C₆ alkyl-SOR²⁰, C₁-C₆ alkyl-SO₂R²⁰, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁴ is selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR aryl, heteroaryl, heterocyclyl, SR₁₃, C₁-C₆ alkyl-CC₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁵ is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally-substituted with one or more of the groups defined by R²¹;
R¹⁶ and R¹⁷ are each independently selected from —H, C₁-C₆ alkyl, C₂-C₆ alkenyl, C₂-C₆ alkynyl, amino, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁶, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R¹⁸, R¹⁹ are each independently selected from —H, C₁-C₆ alkyl, C₂-C₆ alkenyl, C₂-C₆ alkynyl, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁷, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²⁰ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, O—R²⁴, C₁-C₄ alkyl-OR²⁴, aryl, heteroaryl, heterocyclyl, SR²⁴, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, arylalkoxyalkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²¹ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, nitro, cyano, O—R²⁴, C₁-C₄ alkyl-OR²⁶, aryl, heteroaryl, heterocyclyl, COR²⁶, SR²⁴, SOR²⁶, SO₂R²²⁶, C₁-C₆ alkyl-COR^{a}-R CC₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²² and R²³ are each independently selected from —H, C₁-C₆ alkyl, C₂-C₆ alkenyl, C₂-C₆ alkynyl, amino, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁴ and R²⁵ are each independently selected from —H, C₁-C₆ alkyl, C₂-C₆ alkenyl, C₂-C₆ alkynyl, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁶ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, O—R³⁰, C₁-C₄ alkyl-OR³⁰, aryl, heteroaryl, heterocyclyl, SR³⁰, C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁷ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, nitro, cyano, O—R³⁰, C₁-C₄ alkyl-OR³⁰, aryl, heteroaryl, heterocyclyl, COR³², SR³⁰, SOR³², SO₂R³², C₁-C₆ alkyl-COR³², C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁸ and R²⁹ are each independently selected from —H, C₁-C₆ alkyl, C₂-C₆ alkenyl, C₂-C₆ alkynyl, amino, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁰ and R³¹ are each independently selected from —H, C₁-C₆ alkyl, C₂-C₆ alkenyl, C₂-C₆ alkynyl, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³² is selected from —H, C₁-C₆ alkyl, C₂-C₆ alkenyl, C₂-C₆ alkynyl, amino, NHR³⁴, NR³⁴R³⁵, C₁-C₆ alkyl-NHR³⁴, C₁-C₆ alkyl-NR³⁴R³⁵, O—R³⁶, C₁-C₄ alkyl-OR³⁶, aryl, heteroaryl, heterocyclyl, SR³⁶, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₃-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³³ is selected from —H, C₁-C₆ alkyl, C₂-C₆ alkenyl, C₂-C₆ alkynyl, amino, NHR³⁴, NR³⁴R³⁵, C₁-C₆ alkyl-NHR³¹, C₁-C₆ alkyl-NR³⁴R³⁵, nitro, cyano, O—R³⁶, C₁-C₄ alkyl-OR³⁶, aryl, heteroaryl, heterocyclyl, COR³⁸, SR³⁵, SOR³⁸, SO₂R³⁸, C₁-C₆ alkyl-COR³⁸, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl-are optionally substituted with one or more of the groups defined by R³⁹;
R³⁴, R³⁵, R³⁶ and R³⁷ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁸ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁹ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, arylalkenyl, heterocyclylalkenyl, arylheterocyclylalkenyl, heterocyclylarylalkenyl, or heteroarylalkyl; and
R² and R³ optionally join to form a saturated or unsaturated ring of 5, 6, 7, or 8 atoms, where the atoms in the ring are independently selected from CR⁴⁰, C(R⁴⁰)₂, C═O, N, NR⁴⁰, O, S, C═S, S═O, or SO₂.

5. A cyclic pyrazole MK-2 inhibiting compound having the structure: 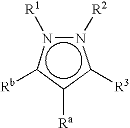
where:
R^{a} and R^{b} join to form a ring structure selected from the group consisting of: 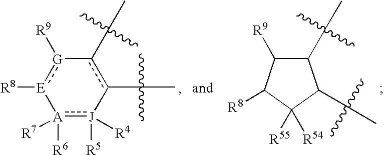
A, E, G and J are carbon, or optionally, one of A, E, G and J is selected from nitrogen, sulfur, or oxygen;
when A, E, G, or J is oxygen or sulfur, no substituent groups are bonded to the oxygen or the sulfur;
when A, E, G, or J is nitrogen, the nitrogen is optionally unsubstituted, or is substituted with R⁹;
each of R¹, R², R⁴ and R⁶ is optionally absent, or is independently selected from the R³ groups;
R³, R⁵, R⁷, R⁴⁰, R⁴⁵, R⁴⁶, R⁴⁷, R⁴⁸, R⁵⁴ and R⁵⁵ are each independently selected from —H, C₁-C₆ alkyl, C₂-C₆ alkenyl, C₂-C₆ alkynyl, nitro C₁-C₆ alkyl, azido C₁-C₆ alkyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁵;
R⁸ and R⁹ join to form the ring structure: 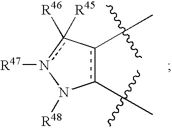
R¹⁰, R¹¹, are each independently selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, CO₂R¹⁹, COR²⁰, CONHR¹⁹, CON(R¹⁹)₂, SR¹³, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, arylaryl, arylheteroaryl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹², R¹³ are each independently selected from —H, C₁-C₆ alkyl, C₂-C₆ alkenyl, C₂-C₆ alkynyl, C₁-C₆ alkyl-NHR¹⁶, C₁-C₆ alkyl-NR¹⁶R¹⁷, C₁-C₄ alkyl-OR¹⁸, aryl, heteroaryl, heterocyclyl, CO₂R¹⁹, COR²⁰, CONHR¹⁹, CON(R¹⁹)₂, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁴ is selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹³, aryl, heteroaryl, heterocyclyl, SR¹⁸, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁵ is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁶ and R¹⁷ are each independently selected from —H, C₁-C₆ alkyl, C₂-C₆ alkenyl, C₂-C₆ alkynyl, amino, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁶, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R¹⁸, R¹⁹ are each independently selected from —H, C₁-C₆ alkyl, C₂-C₆ alkenyl, C₂-C₆ alkynyl, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁷, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²⁰ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, O—R²⁴, C₁-C₄ alkyl-OR²⁴, aryl, heteroaryl, heterocyclyl, SR²⁴, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, arylalkoxyalkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²¹ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, nitro, cyano, O—R²⁴, C₁-C₄ alkyl-OR²⁴, aryl, heteroaryl, heterocyclyl, COR²⁶, SR²⁴, SOR²⁶, SO₂R²⁶, C₁-C₆ alkyl-COR²⁶, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²² and R²³ are each independently selected from —H, C₁-C₆ alkyl, C₂-C₆ alkenyl, C₂-C₆ alkynyl, amino, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁴ and R²⁵ are each independently selected from —H, C₁-C₆ alkyl, C₂-C₆ alkenyl, C₂-C₆ alkynyl, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁶ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, O—R³⁰, C₁-C₄ alkyl-OR³⁰, aryl, heteroaryl, heterocyclyl, SR³⁰, C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁷ is selected from —H, C₁-C₆ alkyl, C₂-C₆ alkenyl, C₂-C₆ alkynyl, amino, NHR²¹, NR²⁸R²⁹, C₁-C₆ alkyl-NHR²⁸, C₁-C₆ alkyl-NR²⁸R²⁹, nitro, cyano, O—R³⁰, C₁-C₄ alkyl-OR³⁰, aryl, heteroaryl, heterocyclyl, COR³², SR³⁰, SOR³², SO₂R³², C₁-C₆ alkyl-COR³², C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁸ and R²⁹ are each independently selected from —H, C₁-C₆ alkyl, C₂-C₆ alkenyl, C₂-C₆ alkynyl, amino, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁰ and R³¹ are each independently selected from —H, C₁-C₆ alkyl, C₂-C₆ alkenyl, C₂-C₆ alkynyl, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³² is selected from —H, C₁-C₆ alkyl, C₂-C₆ alkenyl, C₂-C₆ alkynyl, amino, NHR³⁴, NR³⁴R³⁵, C₁-C₆ alkyl-NHR³⁴, C₁-C₆ alkyl-NR³⁴R³⁵, O—R³⁶, C₁-C₄ alkyl-OR³⁶, aryl, heteroaryl, heterocyclyl, SR³⁶, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³³ is selected from —H, C₁-C₆ alkyl, C₂-C₆ alkenyl, C₂-C₆ alkynyl, amino, NHR³⁴, NR³⁴R³⁵, C₁-C₆ alkyl-NHR³¹, C₁-C₆ alkyl-NR³⁴R³⁵, nitro, cyano, O—R³⁶, C₁-C₄ alkyl-OR³⁶, aryl, heteroaryl, heterocyclyl, COR³⁸, SR³⁵, SOR³⁸, SO₂R³⁸, C₁-C₆ alkyl-COR³⁸, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁴, R³⁵, R³⁶ and R³⁷ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁸ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁹ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, arylalkenyl, heterocyclylalkenyl, arylheterocyclylalkenyl, heterocyclylarylalkenyl, or heteroarylalkyl; and
R² and R³ optionally join to form a saturated or unsaturated ring of 5, 6, 7, or 8 atoms, where the atoms in the ring are independently selected from CR⁴⁰, C(R⁴⁰)₂, C═O, N, NR⁴⁰, O, S, C═S, S═O, or SO₂.

6. A cyclic pyrazole MK-2 inhibiting compound having the structure: 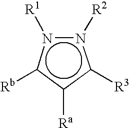
where:
R^{a} and R^{b} join to form a ring structure selected from the group consisting of: 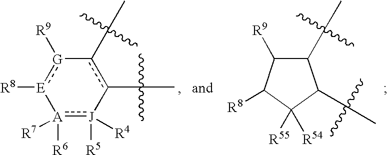
A, E, G and J are carbon, or optionally, one of A, E, G and J is selected from nitrogen, sulfur, or oxygen;
when A, E, G, or J is oxygen or sulfur, no substituent groups are bonded to the oxygen or the sulfur;
when A, E, G, or J is nitrogen, the nitrogen is optionally unsubstituted, or is substituted with R⁹;
each of R¹, R², R⁴ and R⁶ is optionally absent, or is independently selected from the R³ groups;
R³, R⁵, R⁷, R⁴⁰, R⁴⁹, R⁵⁰, R⁵¹, R⁵⁴ and R⁵⁵ are each independently selected from —H, C₁-C₆ alkyl, C₂-C₆ alkenyl, C₂-C₆ alkynyl, nitro C₁-C₆ alkyl, azido C₁-C₆ alkyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁵;
R⁸ and R⁹ join to form the ring structure: 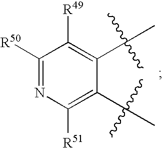
R¹⁰, R¹¹, are each independently selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, CO₂R¹⁹, COR²⁰, CONHR¹⁹, CON(R¹⁹)₂, SR¹⁸, SOR²⁰, SO₂R²⁰, C₁-C₆alkyl-CO₂R C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, arylaryl, arylheteroaryl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹², R¹³ are each independently selected from —H, C₁-C₆ alkyl, C₂-C₆ alkenyl, C₂-C₆ alkynyl, C₁-C₆ alkyl-NHR¹⁶, C₁-C₆ alkyl-NR¹⁶R¹⁷, C₁-C₄ alkyl-OR¹⁸, aryl, heteroaryl, heterocyclyl, CO₂R¹⁹, COR²⁰, CONHR¹⁹, CON(R¹⁹)₂, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁴ is selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, SR¹⁸, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁵ is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁶ and R¹⁷ are each independently selected from —H, C₁-C₆ alkyl, C₂-C₆ alkenyl, C₂-C₆ alkynyl, amino, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁶, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R¹⁸, R¹⁹ are each independently selected from —H, C₁-C₆ alkyl, C₂-C₆ alkenyl, C₂-C₆ alkynyl, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁷, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²⁰ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, O—R²⁴, C₁-C₄ alkyl-OR²⁴, aryl, heteroaryl, heterocyclyl, SR²⁴, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, arylalkoxyalkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²¹ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, nitro, cyano, O—R²⁴, C₁-C₄ alkyl-OR²⁴, aryl, heteroaryl, heterocyclyl, COR²⁶, SR²⁴, SOR²⁶, SO₂R²⁶, C₁-C₆ alkyl-COR²⁶, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²² and R²³ are each independently selected from —H, C₁-C₆ alkyl, C₂-C₆ alkenyl, C₂-C₆ alkynyl, amino, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³¹, C₁-C₆-alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁴ and R²⁵ are each independently selected from —H, C₁-C₆ alkyl, C₂-C₆ alkenyl, C₂-C₆ alkynyl, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁶ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, O—R³⁰, C₁-C₄ alkyl-OR³⁰, aryl, heteroaryl, heterocyclyl, SR³⁰, C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁷ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, nitro, cyano, O—R³⁰, C₁-C₄alkyl-OR³⁰, aryl, heteroaryl, heterocyclyl, COR³², SR³⁰, SOR³², SO₂R³², C₁-C₆ alkyl-COR³², C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁸ and R²⁹ are each independently selected from —H, C₁-C₆ alkyl, C₂-C₆ alkenyl, C₂-C₆ alkynyl, amino, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁰ and R³¹ are each independently selected from —H, C₁-C₆ alkyl, C₂-C₆ alkenyl, C₂-C₆ alkynyl, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³² is selected from —H, C₁-C₆ alkyl, C₂-C₆ alkenyl, C₂-C₆ alkynyl, amino, NHR³⁴, NR³⁴R³⁵, C₁-C₆ alkyl-NHR³⁴, C₁-C₆ alkyl-NR³⁴R³⁵, O—R³⁶, C₁-C₄ alkyl-OR³⁵, aryl, heteroaryl, heterocyclyl, SR³⁶, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³³ is selected from —H, C₁-C₆ alkyl, C₂-C₆ alkenyl, C₂-C₆ alkynyl, amino, NHR³⁴, NR³⁴R³⁵, C₁-C₆ alkyl-NHR³¹, C₁-C₆ alkyl-NR³⁴R³⁵, nitro, cyano, O—R³⁶, C₁-C₄ alkyl-OR³⁶, aryl, heteroaryl, heterocyclyl, COR³⁸, SR³⁵, SOR³⁸, SO₂R³⁸, C₁-C₆ alkyl-COR³⁸, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁴, R³⁵, R³⁶ and R³⁷ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁸ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and-bicyclic cycloalkyl, wherein aryl, heteroaryl, heterocyclyl, mono- and bicyclic cycloalkyl are optionally substituted with one or more of the groups defined by R³⁹;
R³⁹ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, arylalkenyl, heterocyclylalkenyl, arylheterocyclylalkenyl, heterocyclylarylalkenyl, or heteroarylalkyl; and
R² and R³ optionally join to form a saturated or unsaturated ring of 5, 6, 7, or 8 atoms, where the atoms in the ring are independently selected from CR⁴⁰, C(R⁴⁰)₂, C═O, N, NR⁴⁰, O, S, C═S, S═O, or SO₂.

7. A cyclic pyrazole MK-2 inhibiting compound having the structure: 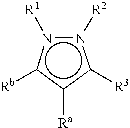
where:
R^{a} and R^{b} join to form a ring structure selected from the group consisting of: 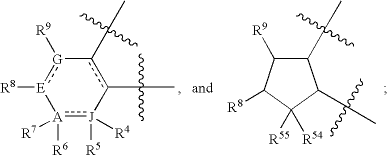
A, E, G and J are carbon, or optionally, one of A, E, G and J is selected from nitrogen, sulfur, or oxygen;
when A, E, G, or J is oxygen or sulfur, no substituent groups are bonded to the oxygen or the sulfur;
when A, E, G, or J is nitrogen, the nitrogen is optionally unsubstituted, or is substituted with R⁹;
each of R¹, R², R⁴ and R⁶ is optionally absent, or is independently selected from the R³ groups;
R³, R⁵, R⁷, R⁴⁰, R⁵², R⁵³, R⁵⁴ and R⁵⁵ are each independently selected from —H, C₁-C₆ alkyl, C₂-C₆ alkenyl, C₂-C₆ alkynyl, nitro C₁-C₆ alkyl, azido C₁-C₆ alkyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁵;
R⁸ and R⁹ join to form a ring structure selected from: 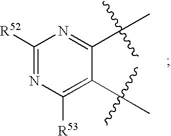
R¹⁰, R¹¹, are each independently selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁵, aryl, heteroaryl, heterocyclyl, CO₂R¹⁹, COR²⁰, CONHR¹⁹, CON(R¹⁹)₂, SR¹⁸, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, arylaryl, arylheteroaryl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹², R¹³ are each independently selected from —H, C₁-C₆ alkyl, C₂-C₆ alkenyl, C₂-C₆ alkynyl, C₁-C₆ alkyl-NHR¹⁶, C₁-C₆ alkyl-NR¹⁶R¹⁷, C₁-C₄ alkyl-OR¹⁸, aryl, heteroaryl, heterocyclyl, CO₂R¹⁹, COR²⁰, CONHR¹⁹, CON(R¹⁹)₂, SOR²⁰, SO₂R²⁰, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁴ is selected from —H, C₁-C₆ alkyl, C₂-C₆ alkenyl, C₂-C₆ alkynyl, amino, NHR¹⁶, NR¹⁶R¹⁷, C₁-C₆ alkyl-NHR¹⁶, C₁-C₆ alkyl-NR¹⁶R¹⁷, O—R¹⁸, C₁-C₄ alkyl-OR¹⁸, aryl, heteroaryl, heterocyclyl, SR¹⁸, C₁-C₆ alkyl-CO₂R¹⁹, C₁-C₆ alkyl-COR²⁰, C₁-C₆ alkyl-CONHR¹⁹, C₁-C₆ alkyl-CON(R¹⁹)₂, C₁-C₆ alkyl-SR¹⁸, C₁-C₆ alkyl-SOR²⁰, C₁-C₆ alkyl-SO₂R²⁰, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁵ is selected from —H, C₁-C₆ alkyl, C₂-C₆ alkenyl, C₂-C₆ alkynyl, amino, NHR¹⁰, NR¹¹R¹², C₁-C₆ alkyl-NHR¹⁰, C₁-C₆ alkyl-NR¹¹R¹², nitro, cyano, O—R¹³, C₁-C₄ alkyl-OR¹³, aryl, heteroaryl, heterocyclyl, COR¹⁴, SR¹³, SOR¹⁴, SO₂R¹⁴, C₁-C₆ alkyl-COR¹⁴, C₁-C₆ alkyl-SR¹³, C₁-C₆ alkyl-SOR¹⁴, C₁-C₆ alkyl-SO₂R¹⁴, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²¹;
R¹⁶ and R¹⁷ are each independently selected from —H, C₁-C₆ alkyl, C₂-C₆ alkenyl, C₂-C₆ alkynyl, amino, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁶, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R¹⁸, R¹⁹ are each independently selected from —H, C₁-C₆ alkyl, C₂-C₆ alkenyl, C₂-C₆ alkynyl, C₁-C₆ alkyl-NHR²², C₁-C₆ alkyl-NR²²R²³, C₁-C₄ alkyl-OR²⁴, aryl, heteroaryl, heterocyclyl, CO₂R²⁵, COR²⁶, CONHR²⁵, CON(R²⁵)₂, SOR²⁶, SO₂R²⁷, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²⁰ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, O—R²⁴, C₁-C₄ alkyl-OR²⁴, aryl, heteroaryl, heterocyclyl, SR²⁴, C₁-C₆ alkyl-CO₂R²⁵, C₁-C₆ alkyl-COR²⁶, C₁-C₆ alkyl-CONHR²⁵, C₁-C₆ alkyl-CON(R²⁵)₂, C₁-C₆ alkyl-SR²⁴, C₁-C₆ alkyl-SOR²⁶, C₁-C₆ alkyl-SO₂R²⁶, halo, halo C₁-C₆ alkyl, arylalkoxyalkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²¹ is selected from —H, C₁-C₆ alkyl, C₂-C₆ alkenyl, C₂-C₆ alkynyl, amino, NHR²², NR²²R²³, C₁-C₆ alkyl-NHR²², C₁-C₆ alkyl-NR²²R²³, nitro, cyano, O—R²⁴, C₁-C₄ alkyl-OR²⁴, aryl, heteroaryl, heterocyclyl, COR²⁶, SR²⁴, SOR²⁶, SO₂R²⁶, C₁-C₆ alkyl-COR²⁶, C₁-C₆ alkyl-SR²⁴, C₁-C₆ alkyl-SOR²⁶, C₁-C₆ alkyl-SO₂R²⁶, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁷;
R²² and R²³ are each independently selected from —H, C₁-C₆ alkyl, C₂-C₆ alkenyl, C₂-C₆ alkynyl, amino, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups-defined by R³³;
R²⁴ and R²⁵ are each independently selected from —H, C₁-C₆ alkyl, C₂-C₆ alkenyl, C₂-C₆ alkynyl, C₁-C₆ alkyl-NHR²⁸, C₁-C₆ alkyl-NR²⁸R²⁹, C₁-C₄ alkyl-OR³⁰, aryl, heteroaryl, heterocyclyl, CO₂R³¹, COR³², CONHR³¹, CON(R³¹)₂, SOR³², SO₂R³², C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁶ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, O—R³⁰, C₁-C₄ alkyl-OR³⁰, aryl, heteroaryl, heterocyclyl, SR³⁰, C₁-C₆ alkyl-CO₂R³¹, C₁-C₆ alkyl-COR³², C₁-C₆ alkyl-CONHR³¹, C₁-C₆ alkyl-CON(R³¹)₂, C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁷ is selected from —H, C₁-C₆ alkyl, C₂-C₆ alkenyl, C₂-C₆ alkynyl, amino, NHR²⁸, NR²⁸R²⁹, C₁-C₆ alkyl-NHR²⁸, C₁-C₆ alkyl-NR²⁸R²⁹, nitro, cyano, O—R³⁰, C₁-C₄ alkyl-OR³⁰, aryl, heteroaryl, heterocyclyl, COR³², SR³⁰, SOR³², SO₂R³², C₁-C₆ alkyl-COR³², C₁-C₆ alkyl-SR³⁰, C₁-C₆ alkyl-SOR³², C₁-C₆ alkyl-SO₂R³², halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³³;
R²⁸ and R²⁹ are each independently-selected from —H, —C₁-C₆ alkyl, C₂-C₆ alkenyl, C₂-C₆ alkynyl, amino, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁰ and R³¹ are each independently selected from —H, C₁-C₆ alkyl, C₂-C₆ alkenyl, C₂-C₆ alkynyl, C₁-C₆ alkyl-NHR³⁴, C₁-C₆ alkyl-NR³⁴R³⁵, C₁-C₄ alkyl-OR³⁶, aryl, heteroaryl, heterocyclyl, CO₂R³⁷, COR³⁸, CONHR³⁷, CON(R³⁷)₂, SOR³⁸, SO₂R³⁸, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo C₁-C₆ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³² is selected from —H, C₁-C₆ alkyl, C₂-C₆ alkenyl, C₂-C₆ alkynyl, amino, NHR³⁴, NR³⁴R³⁵, C₁-C₆ alkyl-NHR³⁴, C₁-C₆ alkyl-NR³⁴R³⁵, O—R³⁶, C₁-C₄ alkyl-OR³⁶, aryl, heteroaryl, heterocyclyl, SR³⁶, C₁-C₆ alkyl-CO₂R³⁷, C₁-C₆ alkyl-COR³⁸, C₁-C₆ alkyl-CONHR³⁷, C₁-C₆ alkyl-CON(R³⁷)₂, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³³ is selected from —H, C₁-C₆ alkyl, C₂-C₆ alkenyl, C₂-C₆ alkynyl, amino, NHR³⁴, NR³⁴R³⁵, C₁-C₆ alkyl-NHR³¹, C₁-C₆ alkyl-NR³⁴R³⁵, nitro, cyano, O—R³⁶, C₁-C₄ alkyl-OR³⁶, aryl, heteroaryl, heterocyclyl, COR³⁸, SR³⁵, SOR³⁸, SO₂R³⁸, C₁-C₆ alkyl-COR³⁸, C₁-C₆ alkyl-SR³⁶, C₁-C₆ alkyl-SOR³⁸, C₁-C₆ alkyl-SO₂R³⁸, halo, halo C₁-C₆ alkyl, alkylaryl, alkylheterocyclyl, alkylheteroaryl, arylalkyl, heteroarylalkyl, heterocyclylalkyl, arylalkenyl, heterocyclylalkenyl, arylheterocyclylalkenyl, heterocyclylarylalkenyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁴, R³⁵, R³⁶ and R³⁷ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁸ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁹;
R³⁹ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, arylalkenyl, heterocyclylalkenyl, arylheterocyclylalkenyl, heterocyclylarylalkenyl, or heteroarylalkyl; and
R² and R³ optionally join to form a saturated or unsaturated ring of 5, 6, 7, or 8 atoms, where the atoms in the ring are independently selected from CR⁴⁰, C(R⁴⁰)₂, C═O, N, NR⁴⁰, O, S, C═S, S═O, or SO₂.

8. A cyclic pyrazole MK-2 inhibiting compound having the structure: 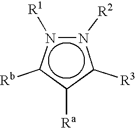
where:
R^{a} and R^{b} join to form the ring structure: 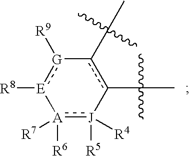
A, E, J and G are carbon;
R⁴, R⁵, and R⁶ are hydrogen;
R⁷ is selected from hydrogen or alkyl;
R⁸ and R⁹ join to form a ring structure that is selected from: 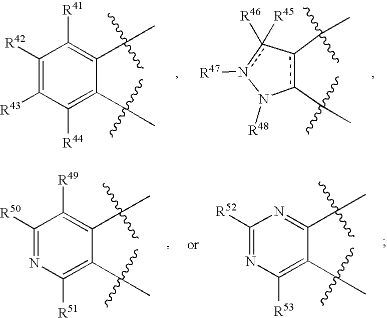
R¹ is optionally absent, or is selected from alkyl, alkenyl, haloalkyl, hydroxyalkyl, carbamylaryl, or arylalkoxycarbonylpiperidyl;
R² is optionally absent, or is selected from hydrogen, alkyl, alkenyl, aminoalkyl, alkoxyarylalkylaminoalkyl, alkylarylalkylaminoalkyl, haloalkylarylarylalkylaminoalkyl, nitroarylalkylaminoalkyl, aminoalkylarylarylalkylaminoalkyl, alkylsulfonylarylalkylaminoalkyl, alkylcarbonylarylalkylaminoalkyl, benzothienylarylalkylaminoalkyl, alkylfurylalkylaminoalkyl, cyanoarylalkylaminoalkyl, halothienylalkylaminoalkyl, haloarylalkylaminoalkyl, pyridylarylalkylaminoalkyl, haloarylalkylsulfonylaminoalkyl, arylalkylsulfonylaminoalkyl, arylarylalkylaminoalkyl, thienylalkylaminoalkyl, alkylarylarylalkylaminoalkyl, alkylarylarylalkylaminoalkyl, haloarylarylalkylaminoalkyl, haloalkylarylarylalkylaminoalkyl, furylarylalkylaminoalkyl, carboxylarylarylalkylaminoaryl, hydroxyalkyl, alkoxy(halo)arylalkylaminoalkyl, isoquinolinylaminoalkyl, hydroxyalkylcarbonylaminoalkyl, alkylaminoalkyl, alkoxycarbonylaminoalkyl, pyridylalkylaminoalkyl, aminoalkylarylaminoalkyl, alkenylaminoalkyl, aminoarylalkylaminoalkyl, alkoxyarylaminocarbonylaminoalkyl, cyclopropylalkylaminoalkyl, pyrroylalkylaminoalkyl, furylalkylaminoalkyl, piperidylalkyl, imidazolylalkylaminoalkyl, aminoarylaminoalkyl, pyridylcarbonylaminoalkyl, cyclopentylaminoalkyl, isoquinolinylalkylaminoalkyl, isoquinolinylamino(hydroxy)alkyl, pyrroylalkylaminoalkyl, arylalkylaminocarbonylaminoalkyl, cyanoalkyl, cyanoarylaminocarbonylaminoalkyl, haloalkyl, carboxy(amino)alkyl, aminocarbonylaminoalkyl, alkylsulfonylaminoalkyl, alkoxyarylalkylcarbonylaminoalkyl, thienylalkylamino(hydroxy)alkyl, cyano(amino)alkyl, pyridylaminoalkyl, alkoxycarbonylaminocarbonylaminoalkyl, carbamylpiperidylalkyl, carbamylalkyl, piperidyl, or arylalkylaminoalkyl;
R³ is selected from hydrogen, carboxyl, alkoxycarbonyl, carbamide, tetrazolyl, alkylthio, or R² and R³ optionally join to form a 6 or 7 membered heterocycle having 2 nitrogen atoms in the ring and with at least one ring nitrogen or carbon being substituted with a group selected from hydrogen, alkyl, oxo, haloalkyl, thienylalkylaminoalkyl, hydroxyalkyl, azidoalkyl, haloarylalkylaminoalkyl, nitroalkyl, carbamyl, alkoxycarbonyl, haloarylalkylsulfonylamino, or aminoalkyl;
R⁴¹, R⁴⁹ and R⁵³ are hydrogen;
R⁴² is selected from hydrogen, nitroaryl, haloalkyl, haloaryl, cyanoaryl, nitroaryl, alkylsulfonylaryl, aminoaryl, alkylamino, alkylaryl, pyridyl, carboxyaryl, arylalkyl, hydroxyl, amino, carboxyalkyl, alkyl, haloarylalkyl, aminosulfonyl, imidazolyl, indazolyl, pyridyl, indolyl, pyrazolyl, or halo;
R⁴³ is selected from hydroxy or alkoxy;
R⁴⁴ is selected from hydrogen or halo;
R⁴⁵ and R⁴⁷ are absent;
R⁴⁶ is selected from hydrogen, arylalkylthio, cyanoalkylthio, alkenylthio, imidazolyl, indazolyl, pyridyl, indolyl, pyrazolyl, or alkylthio;
R⁴⁸ is selected from hydrogen or arylalkyl;
R⁴⁹ and R⁵¹ are hydrogen;
R⁵⁰ is selected from hydrogen, halo, alkyl, benzodioxol, alkoxy, alkylaminoalkyl, isoquinolinyl, nitroaryl, alkoxyaryl, alkylaminoaryl, arylalkyl, hydroxyaryl, quinolinyl, hydroxyarylalkenyl, alkylaryl, haloaryl, haloalkylaryl, pyrazolyl, alkylsulfonylaryl, cyanoaryl, benzofuranyl, arylamino, haloarylalkyl, alkenyl, alkoxy, aminoaryl, haloarylalkenyl, alkoxyarylalkenyl, alkylthio, heterocyclyl, alkoxyarylalkylamino, arylalkylamino, hydroxypiperidyl, pyrrolidyl, tetrahydropyrazinoyl, arylalkoxyaryl, aminoalkylamino, cyano, piperizinyl, alkylpiperidinyl, alkylpiperizinyl, imidazolyl, indazolyl, pyridyl, indolyl, pyrazolyl, or naphthyl;
R⁵¹ is selected from hydrogen, aryl, halo, alkyl, arylamino, alkylthio, or alkoxy;
R⁵² is selected from hydrogen, quinolinyl, benzodioxyl, alkoxyaryl, morpholinyl, dihydroisoquinolinyl, imidazolyl, indazolyl, pyridyl, indolyl, pyrazolyl, or aryl; and
R⁵³ is selected from hydrogen, or halo.

9. A cyclic pyrazole MK-2 inhibiting compound having the structure: 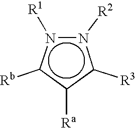
where:
R^{a} and R^{b} join to form the ring structure: 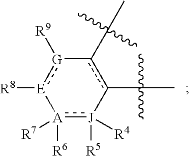
A, E, J and G are carbon;
R⁴, R⁵, and R⁶ are hydrogen;
R⁷ is selected from hydrogen or alkyl;
R⁸ and R⁹ join to form a ring structure that is selected from: 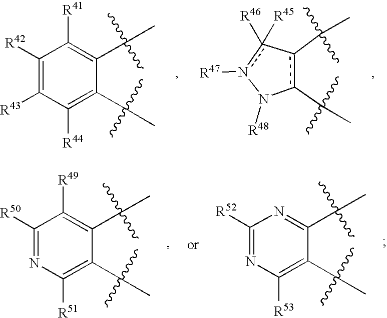
R¹ is absent, or is optionally arylalkoxycarbonylpiperidyl;
R² is selected from hydrogen, alkyl, alkenyl, aminoalkyl, alkoxyarylalkylaminoalkyl, alkylarylalkylaminoalkyl, haloalkylarylarylalkylaminoalkyl, nitroarylalkylaminoalkyl, aminoalkylarylarylalkylaminoalkyl, alkylsulfonylarylalkylaminoalkyl, alkylcarbonylarylalkylaminoalkyl, benzothienylarylalkylaminoalkyl, alkylfurylalkylaminoalkyl, cyanoarylalkylaminoalkyl, halothienylalkylaminoalkyl, haloarylalkylaminoalkyl, pyridylarylalkylaminoalkyl, haloarylalkylsulfonylaminoalkyl, arylalkylsulfonylaminoalkyl, arylarylalkylaminoalkyl, thienylalkylaminoalkyl, alkylarylarylalkylaminoalkyl, alkylarylarylalkylaminoalkyl, haloarylarylalkylaminoalkyl, haloalkylarylarylalkylaminoalkyl, furylarylalkylaminoalkyl, carboxylarylarylalkylaminoaryl, hydroxyalkyl, alkoxy(halo)arylalkylaminoalkyl, isoquinolinylaminoalkyl, hydroxyalkylcarbonylaminoalkyl, alkylaminoalkyl, alkoxycarbonylaminoalkyl, pyridylalkylaminoalkyl, aminoalkylarylaminoalkyl, alkenylaminoalkyl, aminoarylalkylaminoalkyl, alkoxyarylaminocarbonylaminoalkyl, cyclopropylalkylaminoalkyl, pyrroylalkylaminoalkyl, furylalkylaminoalkyl, piperidylalkyl, imidazolylalkylaminoalkyl, aminoarylaminoalkyl, pyridylcarbonylaminoalkyl, cyclopentylaminoalkyl, isoquinolinylalkylaminoalkyl, isoquinolinylamino(hydroxy)alkyl, pyrroylalkylaminoalkyl, arylalkylaminocarbonylaminoalkyl, cyanoalkyl, cyanoarylaminocarbonylaminoalkyl, haloalkyl, carboxy(amino)alkyl, aminocarbonylaminoalkyl, alkylsulfonylaminoalkyl, alkoxyarylalkylcarbonylaminoalkyl, thienylalkylamino(hydroxy)alkyl, cyano(amino)alkyl, pyridylaminoalkyl, alkoxycarbonylaminocarbonylaminoalkyl, carbamylpiperidylalkyl, carbamylalkyl, or arylalkylaminoalkyl;
R³ is selected from carboxyl, alkoxycarbonyl, carbamide, tetrazolyl, or R² and R³ optionally join to form a 6 or 7 membered heterocycle having 2 nitrogen atoms in the ring and with at least one ring nitrogen or carbon being substituted with a group selected from hydrogen, alkyl, oxo, haloalkyl, thienylalkylaminoalkyl, hydroxyalkyl, azidoalkyl, haloarylalkylaminoalkyl, nitroalkyl, carbamyl, alkoxycarbonyl, or aminoalkyl;
R⁴¹, R⁴⁹ and R⁵³ are hydrogen;
R⁴² is selected from hydrogen, haloalkyl, haloaryl, cyanoaryl, nitroaryl, alkylsulfonylaryl, aminoaryl, alkylamino, alkylaryl, carboxyaryl, arylalkyl, hydroxyl, amino, carboxyalkyl, alkyl, imidazolyl, indazolyl, pyridyl, indolyl, pyrazolyl, or halo;
R⁴³ is selected from hydroxy or alkoxy;
R⁴⁴ is selected from hydrogen or halo;
R⁴⁵ and R⁴⁷ are absent;
R⁴⁶ is selected from hydrogen, arylalkylthio, cyanoalkylthio, alkenylthio, imidazolyl, indazolyl, pyridyl, indolyl, pyrazolyl, or alkylthio;
R⁴⁸ is selected from hydrogen or arylalkyl;
R⁴⁹ and R⁵¹ are hydrogen;
R⁵⁰ is selected from hydrogen, halo, alkyl, benzodioxol, alkoxy, alkylaminoalkyl, isoquinolinyl, nitroaryl, alkoxyaryl, alkylaminoaryl, arylalkyl, hydroxyaryl, quinolinyl, hydroxyarylalkenyl, alkylaryl, haloaryl, haloalkylaryl, pyrazolyl, alkylsulfonylaryl, cyanoaryl, benzofuranyl, arylamino, haloalkylaryl, alkenyl, alkoxy, aminoaryl, haloarylalkenyl, alkoxyarylalkenyl, alkylthio, heterocyclyl, alkoxyarylalkylamino, arylalkylamino, hydroxypiperidyl, pyrrolidyl, tetrhydropyrazinoyl, arylalkoxyaryl, imidazolyl, indazolyl, pyridyl, indolyl, pyrazolyl, or naphthyl;
R⁵¹ is selected from hydrogen, halo, alkyl, or alkoxy;
R⁵² is selected from hydrogen, quinolinyl, benzodioxyl, alkoxyaryl, morpholinyl, dihydroisoquinolinyl, imidazolyl, indazolyl, pyridyl, indolyl, pyrazolyl, or aryl; and
R⁵³ is selected from hydrogen or halo.

10. A cyclic pyrazole MK-2 inhibiting compound having the structure: 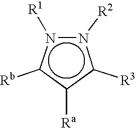
where:
R^{a} and R^{b} join to form the ring structure: 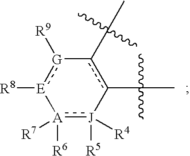
A, E, J and G are carbon;
R⁴, R⁵, and R⁶ are hydrogen;
R⁷ is selected from hydrogen or alkyl;
R⁸ and R⁹ join to form a ring structure that is selected from: 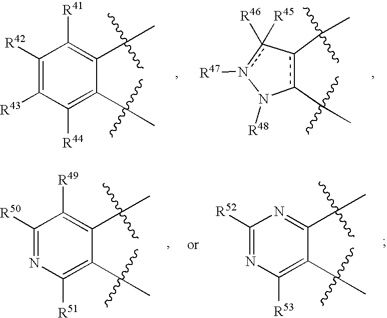
R¹ is absent;
R² is selected from alkyl, alkenyl, aminoalkyl, alkoxyarylalkylaminoalkyl, alkylarylalkylaminoalkyl, haloalkylarylarylalkylaminoalkyl, nitroarylalkylaminoalkyl, aminoalkylarylarylalkylaminoalkyl, alkylsulfonylarylalkylaminoalkyl, alkylcarbonylarylalkylaminoalkyl, benzothienylarylalkylaminoalkyl, alkylfurylalkylaminoalkyl, cyanoarylalkylaminoalkyl, halothienylalkylaminoalkyl, haloarylalkylaminoalkyl, pyridylarylalkylaminoalkyl, haloarylalkylsulfonylaminoalkyl, arylalkylsulfonylaminoalkyl, arylarylalkylaminoalkyl, thienylalkylaminoalkyl, alkylarylarylalkylaminoalkyl, alkylarylarylalkylaminoalkyl, haloarylarylalkylaminoalkyl, haloalkylarylarylalkylaminoalkyl, furylarylalkylaminoalkyl, carboxylarylarylalkylaminoaryl, hydroxyalkyl, alkoxy(halo)arylalkylaminoalkyl, isoquinolinylaminoalkyl, hydroxyalkylcarbonylaminoalkyl, hydrogen, alkylaminoalkyl, alkoxycarbonylaminoalkyl, pyridylalkylaminoalkyl, aminoalkylarylaminoalkyl, alkenylaminoalkyl, aminoarylalkylaminoalkyl, alkoxyarylaminocarbonylaminoalkyl, cyclopropylalkylaminoalkyl, pyrroylalkylaminoalkyl, furylalkylaminoalkyl, piperidylalkyl, imidazolylalkylaminoalkyl, aminoarylaminoalkyl, pyridylcarbonylaminoalkyl, cyclopentylaminoalkyl, isoquinolinylalkylaminoalkyl, isoquinolinylamino(hydroxy)alkyl, pyrroylalkylaminoalkyl, arylalkylaminocarbonylaminoalkyl, cyanoalkyl, or arylalkylaminoalkyl;
R³ is selected from carboxyl, alkoxycarbonyl, carbamide, tetrazolyl, or R² and R³ optionally join to form a 6 or 7 membered heterocycle having 2 nitrogen atoms in the ring and with at least one ring nitrogen or carbon being substituted with a group selected from hydrogen, alkyl, oxo, haloalkyl, thienylalkylaminoalkyl, hydroxyalkyl, azidoalkyl, haloarylalkylaminoalkyl, nitroalkyl, carbamyl, alkoxycarbonyl, or aminoalkyl;
R⁴¹, R⁴⁹ and R⁵³ are hydrogen;
R⁴² is selected from hydrogen, haloalkyl, haloaryl, cyanoaryl, nitroaryl, alkylsulfonylaryl, aminoaryl, alkylamino, alkylaryl, imidazolyl, indazolyl, pyridyl, indolyl, pyrazolyl, or halo;
R⁴³ is hydroxy;
R⁴⁴ is selected from hydrogen or halo;
R⁴⁵ and R⁴⁷ are absent;
R⁴⁶ is selected from hydrogen, arylalkylthio, cyanoalkylthio, alkenylthio, imidazolyl, indazolyl, pyridyl, indolyl, pyrazolyl, or alkylthio;
R⁴⁸, R⁴⁹ and R⁵¹ are hydrogen;
R⁵⁰ is selected from hydrogen, halo, benzodioxol, alkoxy, alkylaminoalkyl, isoquinolinyl, nitroaryl, alkoxyaryl, alkylaminoaryl, arylalkyl, hydroxyaryl, quinolinyl, hydroxyarylalkenyl, alkylaryl, haloaryl, haloalkylaryl, pyrazolyl, alkylsulfonylaryl, cyanoaryl, benzofuranyl, arylamino, haloalkylaryl, alkenyl, alkoxy, aminoaryl, haloarylalkenyl, alkoxyarylalkenyl, alkylthio, heterocyclyl, alkoxyarylalkylamino, arylalkylamino, hydroxypiperidyl, imidazolyl, indazolyl, pyridyl, indolyl, pyrazolyl, or naphthyl;
R⁵¹ is selected from hydrogen, or alkoxy;
R⁵² is selected from hydrogen, quinolinyl, benzodioxyl, alkoxyaryl, morpholinyl, dihydroisoquinolinyl, imidazolyl, indazolyl, pyridyl, indolyl, pyrazolyl, or aryl; and
R⁵³ is selected from hydrogen or halo.

11. A cyclic pyrazole MK-2 inhibiting compound having the structure: 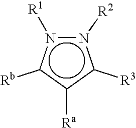
where:
R^{a} and R^{b} join to form the ring structure: 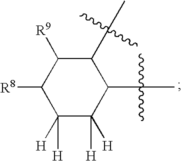
R⁸ and R⁹ join to form a ring structure that is selected from: 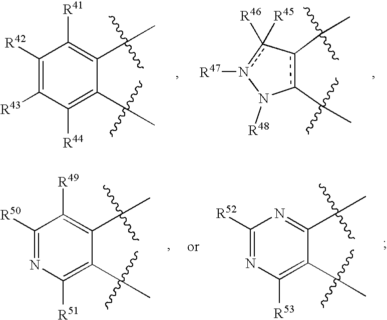
R¹ is absent;
R² is selected from alkyl, aminoalkyl, alkoxyarylalkylaminoalkyl, alkylarylalkylaminoalkyl, haloalkylarylarylalkylaminoalkyl, nitroarylalkylaminoalkyl, aminoalkylarylarylalkylaminoalkyl, alkylsulfonylarylalkylaminoalkyl, alkylcarbonylarylalkylaminoalkyl, benzothienylarylalkylaminoalkyl, alkylfurylalkylaminoalkyl, cyanoarylalkylaminoalkyl, halothienylalkylaminoalkyl, haloarylalkylaminoalkyl, pyridylarylalkylaminoalkyl, haloarylalkylsulfonylaminoalkyl, arylalkylsulfonylaminoalkyl, arylarylalkylaminoalkyl, thienylalkylaminoalkyl, alkylarylarylalkylaminoalkyl, alkylarylarylalkylaminoalkyl, haloarylarylalkylaminoalkyl, haloalkylarylarylalkylaminoalkyl, furylarylalkylaminoalkyl, carboxylarylarylalkylaminoaryl, hydroxyalkyl, alkoxy(halo)arylalkylaminoalkyl, isoquinolinylaminoalkyl, hydroxyalkylcarbonylaminoalkyl, hydrogen, alkylaminoalkyl, alkoxycarbonylaminoalkyl, pyridylalkylaminoalkyl, aminoalkylarylaminoalkyl, alkenylaminoalkyl, aminoarylalkylaminoalkyl, alkoxyarylaminocarbonylaminoalkyl, cyclopropylalkylaminoalkyl, pyrroylalkylaminoalkyl, furylalkylaminoalkyl, piperidylalkyl, imidazolylalkylaminoalkyl, aminoarylaminoalkyl, or arylalkylaminoalkyl;
R³ is selected from carboxyl, alkoxycarbonyl, carbamide, tetrazolyl, or R² and R³ optionally join to form a 6 or 7 membered heterocycle having 2 nitrogen atoms in the ring and with at least one ring nitrogen or carbon being substituted with a group selected from hydrogen, alkyl, oxo, haloalkyl, thienylalkylaminoalkyl, hydroxyalkyl, azidoalkyl, haloarylalkylaminoalkyl, nitroalkyl, carbamyl, or aminoalkyl;
R⁴¹, R⁴⁴, R⁴⁹ and R⁵³ are hydrogen;
R⁴² is selected from hydrogen, haloalkyl, haloaryl, cyanoaryl, nitroaryl, imidazolyl, indazolyl, pyridyl, indolyl, pyrazolyl, or halo;
R⁴³ is hydroxy;
R⁴⁵ and R⁴⁷ are absent;
R⁴⁶ is selected from hydrogen, arylalkylthio, cyanoalkylthio, imidazolyl, indazolyl, pyridyl, indolyl, pyrazolyl, or alkylthio;
R⁴⁸, R⁴⁹, R⁵¹ and R⁵³ are hydrogen;
R⁵⁰ is selected from hydrogen, halo, benzodioxol, alkoxy, alkylaminoalkyl, isoquinolinyl, nitroaryl, alkoxyaryl, alkylaminoaryl, arylalkyl, hydroxyaryl, quinolinyl, hydroxyarylalkenyl, alkylaryl, haloaryl, haloalkylaryl, pyrazolyl, alkylsulfonylaryl, cyanoaryl, benzofuranyl, arylamino, haloalkylaryl, alkenyl, alkoxy, aminoaryl, haloarylalkenyl, alkoxyarylalkenyl, alkylthio, heterocyclyl, alkoxyarylalkylamino, arylalkylamino, hydroxypiperidyl, imidazolyl, indazolyl, pyridyl, indolyl, pyrazolyl, or naphthyl;
R⁵¹ is selected from hydrogen, or alkoxy; and
R⁵² is selected from hydrogen, quinolinyl, benzodioxyl, alkoxyaryl, imidazolyl, indazolyl, pyridyl, indolyl, pyrazolyl, or aryl.

12. A cyclic pyrazole MK-2 inhibiting compound having the structure: 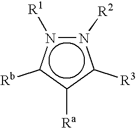
where:
R^{a} and R^{b} join to form the ring structure: 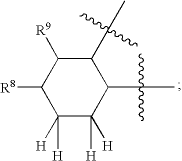
R⁸ and R⁹ join to form a ring structure that is selected from: 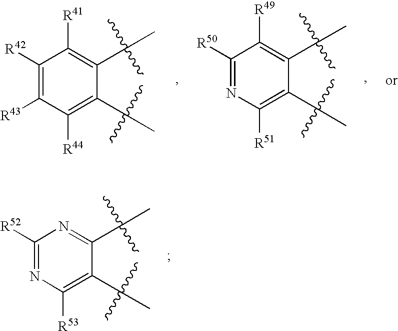
R¹ is absent;
R² is selected from alkyl, aminoalkyl, alkoxyarylalkylaminoalkyl, alkylarylalkylaminoalkyl, haloalkylarylarylalkylaminoalkyl, nitroarylalkylaminoalkyl, aminoalkylarylarylalkylaminoalkyl, alkylsulfonylarylalkylaminoalkyl, alkylcarbonylarylalkylaminoalkyl, benzothienylarylalkylaminoalkyl, alkylfurylalkylaminoalkyl, cyanoarylalkylaminoalkyl, halothienylalkylaminoalkyl, haloarylalkylaminoalkyl, pyridylarylalkylaminoalkyl, haloarylalkylsulfonylaminoalkyl, arylalkylsulfonylaminoalkyl, arylarylalkylaminoalkyl, thienylalkylaminoalkyl, alkylarylarylalkylaminoalkyl, alkylarylarylalkylaminoalkyl, haloarylarylalkylaminoalkyl, haloalkylarylarylalkylaminoalkyl, furylarylalkylaminoalkyl, carboxylarylarylalkylaminoaryl, or hydroxyalkyl;
R³ is selected from carboxyl, carbamide, or R² and R³ optionally join to form a 6 or 7 membered heterocycle having 2 nitrogen atoms in the ring and with at least one ring carbon being substituted with a group selected from hydrogen, oxo, thienylalkylaminoalkyl, hydroxyalkyl, or aminoalkyl;
R⁴¹, R⁴⁴, R⁴⁹ and R⁵³ are hydrogen;
R⁴² is selected from imidazolyl, indazolyl, pyridyl, indolyl, pyrazolyl, or nitroaryl;
R⁴³ is hydroxy;
R⁵⁰ is selected from hydrogen, halo, benzodioxol, alkoxy, alkylaminoalkyl, isoquinolinyl, nitroaryl, alkoxyaryl, alkylaminoaryl, arylalkyl, hydroxyaryl, quinolinyl, hydroxyarylalkenyl, alkylaryl, haloaryl, haloalkylaryl, pyrazolyl, alkylsulfonylaryl, cyanoaryl, imidazolyl, indazolyl, pyridyl, indolyl, pyrazolyl, or naphthyl;
R⁵¹ is selected from hydrogen, or alkoxy; and
R⁵² is selected from hydrogen, quinolinyl, benzodioxyl, imidazolyl, indazolyl, pyridyl, indolyl, pyrazolyl, or aryl.

13. A phenoxy pyrazole MK-2 inhibitor that is selected from the group consisting of:
2-(3-aminopropyl)-7-hydroxy-8-(3-nitrophenyl)-4,5-dihydro-2H-benzo[g]indazole-3-carboxylic acid trifluoroacetate,
2-(2-aminoethyl)-7-hydroxy-8-(3-nitrophenyl)-4,5-dihydro-2H-benzo[g]indazole-3-carboxylic acid trifluoroacetate,
3-hydroxy-2-(3-nitrophenyl)-5,6,8,9,10,11-hexahydro-7H-benzo[g][1,4]diazepino[1,2-b]indazol-7-one trifluoroacetate,
2-(3-aminopropyl)-7-hydroxy-4,5-dihydro-2H-benzo[g]indazole-3-carboxylic acid dihydrochloride,
2-(2-aminoethyl)-8-bromo-7-hydroxy-4,5-dihydro-2H-benzo[g]indazole-3-carboxylic acid trifluoroacetate,
2-(3-aminopropyl)-8-[2-(4-chlorophenyl)ethyl]-7-hydroxy-4,5-dihydro-2H-benzo[g]indazole-3-carboxylic acid trifluoroacetate,
3-hydroxy-2-(3-nitrophenyl)-5,6,9,10-tetrahydrobenzo[g]pyrazino[1,2-b]indazol-7(8H)-one hydrobromide,
2-(3-aminopropyl)-8-bromo-7-hydroxy-4,5-dihydro-2H-benzo[g]indazole-3-carboxylic acid hydrobromide,
2-(3-aminopropyl)-7-hydroxy-8-(4-nitrophenyl)-4,5-dihydro-2H-benzo[g]indazole-3-carboxylic acid trifluoroacetate,
2-(3-aminopropyl)-8-(3-cyanophenyl)-7-hydroxy-4,5-dihydro-2H-benzo[g]indazole-3-carboxylic acid trifluoroacetate,
2-(3-aminopropyl)-7-hydroxy-8-[3-(trifluoromethyl) phenyl]-4,5-dihydro-2H-benzo[g]indazole-3-carboxylic acid trifluoroacetate,
2-(3-aminopropyl)-7-hydroxy-8-(3,3,3-trifluoropropyl)-4,5-dihydro-2H-benzo[g]indazole-3-carboxylic acid trifluoroacetate,
2-(3-aminopropyl)-8-(3-chlorophenyl)-7-hydroxy-4,5-dihydro-2H-benzo[g]indazole-3-carboxylic acid trifluoroacetate,
2-(3-aminopropyl)-7-hydroxy-5-methyl-4,5-dihydro-2H-benzo[g]indazole-3-carboxylic acid trifluoroacetate,
2-(3-aminopropyl)-8-(4-chlorophenyl)-7-hydroxy-4,5-dihydro-2H-benzo[g]indazole-3-carboxylic acid trifluoroacetate,
2-(3-aminopropyl)-7-hydroxy-8-[3-(methylsulfonyl)phenyl]-4,5-dihydro-2H-benzo[g]indazole-3-carboxylic acid trifluoroacetate,
2-(3-aminopropyl)-8-(3,4-difluorophenyl)-7-hydroxy-4,5-dihydro-2H-benzo[g]indazole-3-carboxylic acid trifluoroacetate,
3-hydroxy-5,6,8,9,10,11-hexahydro-7H-benzo[g][1,4]diazepino[1,2-b]indazol-7-one,
8-(3-aminophenyl)-2-(3-aminopropyl)-7-hydroxy-4,5-dihydro-2H-benzo[g]indazole-3-carboxylic acid trifluoroacetate,
6-bromo-7-hydroxy-4,5-dihydro-2H-benzo[g]indazole-3-carboxamide,
2-(3-aminopropyl)-7-hydroxy-8-[4-(methylamino)phenyl]-4,5-dihydro-2H-benzo[g]indazole-3-carboxylic acid trifluoroacetate,
2-(3-aminopropyl)-8-(4-tert-butylphenyl)-7-hydroxy-4,5-dihydro-2H-benzo[g]indazole-3-carboxylic acid trifluoroacetate,
2-(3-aminopropyl)-7-hydroxy-8-pyridin-4-yl-4,5-dihydro-2H-benzo[g]indazole-3-carboxylic acid trifluoroacetate,
7-hydroxy-4,5-dihydro-2H-benzo[g]indazole-3-carboxylic acid,
2-(3-aminopropyl)-7-hydroxy-8-isobutyl-4,5-dihydro-2H-benzo[g]indazole-3-carboxylic acid trifluoroacetate,
2-(3-aminopropyl)-8-(4-carboxy-3,3-dimethylbutyl)-7-hydroxy-4,5-dihydro-2H-benzo[g]indazole-3-carboxylic acid trifluoroacetate,
8-amino-2-(3-aminopropyl)-7-hydroxy-4,5-dihydro-2H-benzo[g]indazole-3-carboxylic acid,
7,8-dihydroxy-4,5-dihydro-2H-benzo[g]indazole-3-carboxylic acid,
2-(3-aminopropyl)-8-benzyl-7-hydroxy-4,5-dihydro-2H-benzo[g]indazole-3-carboxylic acid trifluoroacetate,
2-(3-aminopropyl)-8-(2-chlorophenyl)-7-hydroxy-4,5-dihydro-2H-benzo[g]indazole-3-carboxylic acid trifluoroacetate,
2-allyl-7-hydroxy-4,5-dihydro-2H-benzo[g]indazole-3-carboxylic acid,
2-{3-[(tert-butoxycarbonyl)amino]propyl}-7-methoxy-8-(4-nitrophenyl)-4,5-dihydro-2H-benzo[g]indazole-3-carboxylic acid,
7-hydroxy-2-methyl-4,5-dihydro-2H-benzo[g]indazole-3-carboxylic acid,
2-(3-aminopropyl)-8-(sec-butylamino)-7-hydroxy-4,5-dihydro-2H-benzo[g]indazole-3-carboxylic acid,
2-(3-aminopropyl)-8-(3-carboxyphenyl)-7-hydroxy-4,5-dihydro-2H-benzo[g]indazole-3-carboxylic acid trifluoroacetate,
2-{3-[(tert-butoxycarbonyl)amino]propyl}-7-methoxy-8-(3-nitrophenyl)-4,5-dihydro-2H-benzo[g]indazole-3-carboxylic acid,
2-{3-[(tert-butoxycarbonyl)amino]propyl}-8-(3-cyanophenyl)-7-methoxy-4,5-dihydro-2H-benzo[g]indazole-3-carboxylic acid,
7-hydroxy-1-methyl-4,5-dihydro-1H-benzo[g]indazole-3-carboxylic acid,
2-{3-[(tert-butoxycarbonyl)amino]propyl}-8-[2-(4-chlorophenyl)ethyl]-7-methoxy-4,5-dihydro-2H-benzo[g]indazole-3-carboxylic acid,
2-{3-[(tert-butoxycarbonyl)amino]propyl}-7-methoxy-8-[3-(trifluoromethyl)phenyl]-4,5-dihydro-2H-benzo[g]indazole-3-carboxylic acid,
2-{3-[(tert-butoxycarbonyl)amino]propyl}-8-(4-tert-butylphenyl)-7-methoxy-4,5-dihydro-2H-benzo[g]indazole-3-carboxylic acid,
2-(3-aminopropyl)-6-chloro-7-hydroxy-8-(3-nitrophenyl)-4,5-dihydro-2H-benzo[g]indazole-3-carboxylic acid,
2-{2-[(tert-butoxycarbonyl)amino]ethyl}-7-methoxy-8-(3-nitrophenyl)-4,5-dihydro-2H-benzo[g]indazole-3-carboxylic acid,
2-amino-3-hydroxy-5,6,8,9,10,11-hexahydro-7H-benzo[g][1,4]diazepino[1,2-b]indazol-7-one trifluoroacetate,
2-{3-[(tert-butoxycarbonyl)amino]propyl}-8-(3,4-difluorophenyl)-7-methoxy-4,5-dihydro-2H-benzo[g]indazole-3-carboxylic acid,
methyl 8-[(dimethylamino)sulfonyl]-7-hydroxy-4,5-dihydro-2H-benzo[g]indazole-3-carboxylate,
7-hydroxy-5-methyl-4,5-dihydro-2H-benzo[g]indazole-3-carboxylic acid,
2-{3-[(tert-butoxycarbonyl)amino]propyl}-8-(4-chlorophenyl)-7-methoxy-4,5-dihydro-2H-benzo[g]indazole-3-carboxylic acid,
2-{3-[(tert-butoxycarbonyl)amino]propyl}-8-(3-chlorophenyl)-7-methoxy-4,5-dihydro-2H-benzo[g]indazole-3-carboxylic acid,
2-(3-aminophenyl)-3-hydroxy-5,6,8,9,10,11-hexahydro-7H-benzo[g][1,4]diazepino[1,2-b]indazol-7-one trifluoroacetate,
2-{3-[(tert-butoxycarbonyl)amino]propyl}-8-(2-chlorophenyl)-7-methoxy-4,5-dihydro-2H-benzo[g]indazole-3-carboxylic acid,
ethyl 6-bromo-7-hydroxy-4,5-dihydro-2H-benzo[g]indazole-3-carboxylate,
2-{3-[(tert-butoxycarbonyl)amino]propyl}-7-methoxy-8-(3,3,3-trifluoropropyl)-4,5-dihydro-2H-benzo[g]indazole-3-carboxylic acid,
2-(3-aminopropyl)-8-(3,5-dimethylisoxazol-4-yl)-7-hydroxy-4,5-dihydro-2H-benzo[g]indazole-3-carboxylic acid trifluoroacetate,
5-allyl-7-hydroxy-4,5-dihydro-1H-benzo[g]indazole-3-carboxamide,
2-{3-[(tert-butoxycarbonyl)amino]propyl}-7-methoxy-8-[3-(methylsulfonyl)phenyl]-4,5-dihydro-2H-benzo[g]indazole-3-carboxylic acid,
2-{3-[(tert-butoxycarbonyl)amino]propyl}-8-[1-(tert-butoxycarbonyl)-1H-pyrrol-2-yl]-7-methoxy-4,5-dihydro-2H-benzo[g]indazole-3-carboxylic acid,
7-hydroxy-4-methyl-4,5-dihydro-2H-benzo[g]indazole-3-carboxylic acid,
5-allyl-7-hydroxy-4,5-dihydro-1H-benzo[g]indazole-3-carboxylic acid,
7-[(2-methoxyethoxy)methoxy]-4,5-dihydro-2H-benzo[g]indazole-3-carboxylic acid,
6-bromo-7-hydroxy-4,5-dihydro-2H-benzo[g]indazole-3-carboxylic acid,
8-benzyl-2-{3-[(tert-butoxycarbonyl)amino]propyl}-7-methoxy-4,5-dihydro-2H-benzo[g]indazole-3-carboxylic acid,
methyl 7,8-dihydroxy-4,5-dihydro-2H-benzo[g]indazole-3-carboxylate,
7-hydroxy-1-methyl-4,5-dihydro-1H-benzo[g]indazole-3-carboxamide,
8-bromo-7-hydroxy-4,5-dihydro-2H-benzo[g]indazole-3-carboxylic acid,
2-{3-[(tert-butoxycarbonyl)amino]propyl}-7-methoxy-8-pyridin-4-yl-4,5-dihydro-2H-benzo[g]indazole-3-carboxylic acid,
7-hydroxy-4-methyl-4,5-dihydro-2H-benzo[g]indazole-3-carboxamide,
7-hydroxy-N-methyl-4,5-dihydro-2H-benzo[g]indazole-3-carboxamide,
7-hydroxy-2-methyl-4,5-dihydro-2H-benzo[g]indazole-3-carboxamide,
2-{3-[(tert-butoxycarbonyl)amino]propyl}-7-hydroxy-4,5-dihydro-2H-benzo[g]indazole-3-carboxylic acid,
7-hydroxy-N-(2-hydroxyethyl)-4,5-dihydro-2H-benzo[g]indazole-3-carboxamide,
2-(3-aminopropyl)-8-(sec-butylamino)-7-methoxy-4,5-dihydro-2H-benzo[g]indazole-3-carboxylic acid,
ethyl 7-methoxy-4,5-dihydro-2H-benzo[g]indazole-3-carboxylate,
7-methoxy-4,5-dihydro-2H-benzo[g]indazole-3-carboxamide,
ethyl 7-hydroxy-1-phenyl-4,5-dihydro-1H-benzo[g]indazole-3-carboxylate,
ethyl 7-hydroxy-1-(4-methoxyphenyl)-4,5-dihydro-1H-benzo[g]indazole-3-carboxylate,
2-allyl-7-[(2-methoxyethoxy)methoxy]-4,5-dihydro-2H-benzo[g]indazole-3-carboxamide,
2-allyl-7-[(2-methoxyethoxy)methoxy]-4,5-dihydro-2H-benzo[g]indazole-3-carboxylic acid,
ethyl 7-methoxy-5-phenyl-4,5-dihydro-1H-benzo[g]indazole-3-carboxylate,
7-[(2-methoxyethoxy)methoxy]-4,5-dihydro-2H-benzo[g]indazole-3-carboxamide,
6-bromo-7-[(2-methoxyethoxy)methoxy]-4,5-dihydro-2H-benzo[g]indazole-3-carboxamide,
6-bromo-7-[(2-methoxyethoxy)methoxy]-4,5-dihydro-2H-benzo[g]indazole-3-carboxylic acid,
ethyl 6-bromo-7-[(2-methoxyethoxy)methoxy]-4,5-dihydro-2H-benzo[g]indazole-3-carboxylate,
ethyl 2-allyl-7-[(2-methoxyethoxy)methoxy]-4,5-dihydro-2H-benzo[g]indazole-3-carboxylate,
7-hydroxy-N,N-dimethyl-4,5-dihydro-2H-benzo[g]indazole-3-carboxamide,
8-bromo-2-{2-[(tert-butoxycarbonyl)amino]ethyl}-7-methoxy-4,5-dihydro-2H-benzo[g]indazole-3-carboxylic acid,
ethyl 8-bromo-2-{2-[(tert-butoxycarbonyl)amino]ethyl}-7-methoxy-4,5-dihydro-2H-benzo[g]indazole-3-carboxylate,
8-bromo-2-{3-[(tert-butoxycarbonyl)amino]propyl}-7-methoxy-4,5-dihydro-2H-benzo[g]indazole-3-carboxylic acid,
8-[(dimethylamino)sulfonyl]-7-hydroxy-4,5-dihydro-2H-benzo[g]indazole-3-carboxylic acid,
ethyl 8-bromo-2-{3-[(tert-butoxycarbonyl)amino]propyl}-7-methoxy-4,5-dihydro-2H-benzo[g]indazole-3-carboxylate,
7-hydroxy-8-[(isopropylamino)sulfonyl]-4,5-dihydro-2H-benzo[g]indazole-3-carboxylic acid,
methyl 7-hydroxy-8-[(isopropylamino)sulfonyl]-4,5-dihydro-2H-benzo[g]indazole-3-carboxylate,
ethyl 8-bromo-7-methoxy-4,5-dihydro-2H-benzo[g]indazole-3-carboxylate,
8-(aminosulfonyl)-7-hydroxy-4,5-dihydro-2H-benzo[g]indazole-3-carboxylic acid,
methyl 7-hydroxy-5-methyl-4,5-dihydro-2H-benzo[g]indazole-3-carboxylate,
methyl 7-methoxy-5-methyl-4,5-dihydro-2H-benzo[g]indazole-3-carboxylate,
methyl 7-methoxy-4-methyl-4,5-dihydro-2H-benzo[g]indazole-3-carboxylate,
ethyl 8-(aminosulfonyl)-7-methoxy-4,5-dihydro-2H-benzo[g]indazole-3-carboxylate,
ethyl 2-(3-aminopropyl)-7-hydroxy-4,5-dihydro-2H-benzo[g]indazole-3-carboxylate dihydrochloride,
ethyl 7-[(2-methoxyethoxy)methoxy]-4,5-dihydro-2H-benzo[g]indazole-3-carboxylate,
5-butyl-7-hydroxy-4,5-dihydro-2H-benzo[g]indazole-3-carboxamide,
5-ethyl-7-hydroxy-4,5-dihydro-2H-benzo[g]indazole-3-carboxamide,
5-butyl-7-hydroxy-4,5-dihydro-2H-benzo[g]indazole-3-carboxylic acid,
5-ethyl-7-hydroxy-4,5-dihydro-2H-benzo[g]indazole-3-carboxylic acid,
5-benzyl-7-hydroxy-4,5-dihydro-1H-benzo[g]indazole-3-carboxamide,
7-hydroxy-5-phenyl-4,5-dihydro-1H-benzo[g]indazole-3-carboxamide,
5-benzyl-7-hydroxy-4,5-dihydro-1H-benzo[g]indazole-3-carboxylic acid,
ethyl 5-allyl-7-methoxy-4,5-dihydro-1H-benzo[g]indazole-3-carboxylate,
ethyl 5-benzyl-7-methoxy-4,5-dihydro-1H-benzo[g]indazole-3-carboxylate,
7-hydroxy-5-phenyl-4,5-dihydro-1H-benzo[g]indazole-3-carboxylic acid,
methyl 2-{3-[(tert-butoxycarbonyl)amino]propyl}-8-(3,4-difluorophenyl)-7-methoxy-4,5-dihydro-2H-benzo[g]indazole-3-carboxylate,
methyl 2-{3-[(tert-butoxycarbonyl)amino]propyl}-7-methoxy-8-[3-(methylsulfonyl)phenyl]-4,5-dihydro-2H-benzo[g]indazole-3-carboxylate,
methyl 2-{3-[(tert-butoxycarbonyl)amino]propyl}-7-methoxy-8-(4-nitrophenyl)-4,5-dihydro-2H-benzo[g]indazole-3-carboxylate,
methyl 2-{3-[(tert-butoxycarbonyl)amino]propyl}-7-methoxy-8-(3,3,3-trifluoropropyl)-4,5-dihydro-2H-benzo[g]indazole-3-carboxylate,
3-methoxy-2-(3-nitrophenyl)-5,6,9,10-tetrahydrobenzo[g]pyrazino[1,2-b]indazol-7(8H)-one,
methyl 2-{3-[(tert-butoxycarbonyl)amino]propyl}-8-(3-cyanophenyl)-7-methoxy-4,5-dihydro-2H-benzo[g]indazole-3-carboxylate,
methyl 2-{2-[(tert-butoxycarbonyl)amino]ethyl}-7-methoxy-8-(3-nitrophenyl)-4,5-dihydro-2H-benzo[g]indazole-3-carboxylate,
2-amino-3-methoxy-5,6,8,9,10,11-hexahydro-7H-benzo[g][1,4]diazepino[1,2-b]indazol-7-one,
methyl 8-amino-2-(3-aminopropyl)-7-methoxy-4,5-dihydro-2H-benzo[g]indazole-3-carboxylate hydrochloride,
2-{3-[(tert-butoxycarbonyl)amino]propyl}-7-methoxy-8-(5-nitropentyl)-4,5-dihydro-2H-benzo[g]indazole-3-carboxylic acid,
2-{3-[(tert-butoxycarbonyl)amino]propyl}-8-(4-carboxy-3,3-dimethylbutyl)-7-methoxy-4,5-dihydro-2H-benzo[g]indazole-3-carboxylic acid,
2-{3-[(tert-butoxycarbonyl)amino]propyl}-8-(3,5-dimethylisoxazol-4-yl)-7-methoxy-4,5-dihydro-2H-benzo[g]indazole-3-carboxylic acid,
methyl 2-{3-[(tert-butoxycarbonyl)amino]propyl}-8-(3,5-dimethylisoxazol-4-yl)-7-methoxy-4,5-dihydro-2H-benzo[g]indazole-3-carboxylate,
2-{3-[(tert-butoxycarbonyl)amino]propyl}-8-(3-carboxyphenyl)-7-methoxy-4,5-dihydro-2H-benzo[g]indazole-3-carboxylic acid,
methyl 2-{3-[(tert-butoxycarbonyl)amino]propyl}-7-methoxy-8-[3-(methoxycarbonyl)phenyl]-4,5-dihydro-2H-benzo[g]indazole-3-carboxylate,
2-{3-[(tert-butoxycarbonyl)amino]propyl}-8-isobutyl-7-methoxy-4,5-dihydro-2H-benzo[g]indazole-3-carboxylic acid,
methyl 2-{3-[(tert-butoxycarbonyl)amino]propyl}-8-(3-chlorophenyl)-7-methoxy-4,5-dihydro-2H-benzo[g]indazole-3-carboxylate,
methyl 2-{3-[(tert-butoxycarbonyl)amino]propyl}-8-(sec-butylamino)-7-methoxy-4,5-dihydro-2H-benzo[g]indazole-3-carboxylate,
methyl 2-{3-[(tert-butoxycarbonyl)amino]propyl}-8-isobutyl-7-methoxy-4,5-dihydro-2H-benzo[g]indazole-3-carboxylate,
methyl 8-benzyl-2-{3-[(tert-butoxycarbonyl)amino]propyl}-7-methoxy-4,5-dihydro-2H-benzo[g]indazole-3-carboxylate,
3-methoxy-2-(3-nitrophenyl)-5,6,8,9,10,11-hexahydro-7H-benzo[g][1,4]diazepino[1,2-b]indazol-7-one,
2-bromo-3-methoxy-5,6,9,10-tetrahydrobenzo[g]pyrazino[1,2-b]indazol-7(8H)-one,
methyl 7-methoxy-6,8-dinitro-4,5-dihydro-2H-benzo[g]indazole-3-carboxylate,
methyl 7-methoxy-8-nitro-4,5-dihydro-2H-benzo[g]indazole-3-carboxylate,
methyl 2-{3-[(tert-butoxycarbonyl)amino]propyl}-7-methoxy-8-nitro-4,5-dihydro-2H-benzo[g]indazole-3-carboxylate,
methyl 8-amino-2-{3-[(tert-butoxycarbonyl)amino]propyl}-7-methoxy-4,5-dihydro-2H-benzo[g]indazole-3-carboxylate,
8-amino-2-(3-aminopropyl)-7-methoxy-4,5-dihydro-2H-benzo[g]indazole-3-carboxylic acid,
methyl 2-{3-[(tert-butoxycarbonyl)amino]propyl}-8-[2-(4-chlorophenyl)ethyl]-7-methoxy-4,5-dihydro-2H-benzo[g]indazole-3-carboxylate,
methyl 2-{3-[(tert-butoxycarbonyl)amino]propyl}-8-[1-(tert-butoxycarbonyl)-1H-pyrrol-2-yl]-7-methoxy-4,5-dihydro-2H-benzo[g]indazole-3-carboxylate,
2-{3-[(tert-butoxycarbonyl)amino]propyl}-7-methoxy-5-methyl-4,5-dihydro-2H-benzo[g]indazole-3-carboxylic acid,
methyl 2-{3-[(tert-butoxycarbonyl)amino]propyl}-8-(2-chlorophenyl)-7-methoxy-4,5-dihydro-2H-benzo[g]indazole-3-carboxylate,
2-(3-aminopropyl)-8-(4-chlorophenyl)-7-methoxy-4,5-dihydro-2H-benzo[g]indazole-3-carboxylic acid hydrochloride,
methyl 2-{3-[(tert-butoxycarbonyl)amino]propyl}-7-methoxy-8-(3-nitrophenyl)-4,5-dihydro-2H-benzo[g]indazole-3-carboxylate,
methyl 2-{3-[(tert-butoxycarbonyl)amino]propyl}-8-(4-tert-butylphenyl)-7-methoxy-4,5-dihydro-2H-benzo[g]indazole-3-carboxylate,
methyl 2-{3-[(tert-butoxycarbonyl)amino]propyl}-7-methoxy-8-[3-(trifluoromethyl)phenyl]-4,5-dihydro-2H-benzo[g]indazole-3-carboxylate,
methyl 2-{3-[(tert-butoxycarbonyl)amino]propyl}-8-(4-chlorophenyl)-7-methoxy-4,5-dihydro-2H-benzo[g]indazole-3-carboxylate,
2-bromo-3-methoxy-9-methyl-5,6,9,10-tetrahydrobenzo[g]pyrazino[1,2-b]indazol-7(8H)-one,
2-bromo-3-hydroxy-9-methyl-5,6,9,10-tetrahydrobenzo[g]pyrazino[1,2-b]indazol-7(8H)-one,
methyl 2-{3-[(tert-butoxycarbonyl)amino]propyl}-8-[4-(dimethylamino)phenyl]-7-methoxy-4,5-dihydro-2H-benzo[g]indazole-3-carboxylate,
2-(2-aminoethyl)-8-bromo-7-methoxy-4,5-dihydro-2H-benzo[g]indazole-3-carboxylic acid hydrochloride,
ethyl 7-hydroxy-1-methyl-4,5-dihydro-1H-benzo[g]indazole-3-carboxylate,
7-hydroxy-1-phenyl-4,5-dihydro-1H-benzo[g]indazole-3-carboxamide,
ethyl 7-hydroxy-2-methyl-4,5-dihydro-2H-benzo[g]indazole-3-carboxylate.

14. A pyrazolyl pyrazole MK-2 inhibitor that is selected from the group consisting of:
2-(3-aminopropyl)-8-(methylthio)-2,4,5,6-tetrahydropyrazolo[3,4-e]indazole-3-carboxylic acid dihydrochloride,
2-(3-aminopropyl)-2,4,5,6-tetrahydropyrazolo[3,4-e]indazole-3-carboxylic acid hydrochloride,
2-(3-{[2-(4-bromophenyl)ethyl]amino}propyl)-2,4,5,6-tetrahydropyrazolo[3,4-e]indazole-3-carboxylic acid hydrochloride,
2-(2-aminoethyl)-2,4,5,6-tetrahydropyrazolo[3,4-e]indazole-3-carboxylic acid hydrochloride,
8-(allylthio)-2-(3-aminopropyl)-2,4,5,6-tetrahydropyrazolo[3,4-e]indazole-3-carboxylic acid dihydrochloride,
2-(3-aminopropyl)-8-(benzylthio)-2,4,5,6-tetrahydropyrazolo[3,4-e]indazole-3-carboxylic acid dihydrochloride,
2-{3-[(2-thien-2-ylethyl)amino]propyl}-2,4,5,6-tetrahydropyrazolo[3,4-e]indazole-3-carboxylic acid,
2-{3-[(2-thien-3-ylethyl)amino]propyl}-2,4,5,6-tetrahydropyrazolo[3,4-e]indazole-3-carboxylic acid hydrochloride,
ethyl 2-(3-{[2-(4-bromophenyl)ethyl]amino}propyl)-2,4,5,6-tetra hydropyrazolo[3,4-e]indazole-3-carboxylate,
2-(4-aminobutyl)-2,4,5,6-tetrahydropyrazolo[3,4-e]indazole-3-carboxylic acid dihydrochloride,
1-(methylthio)-4,5,7,8,9,10-hexahydro[1,4]diazepino[1,2-b]pyrazolo[3,4-g]indazol-6(3H)-one,
1-(allylthio)-4,5,7,8,9,10-hexahydro[1,4]diazepino[1,2-b]pyrazolo[3,4-g]indazol-6(3H)-one,
4,5,7,8,9,10-hexahydro[1,4]diazepino[1.,2-b]pyrazolo[3,4-g]indazol-6(3H)-one,
4,5,8,9-tetrahydro-3H-pyrazino[1,2-b]pyrazolo[3,4-g]indazol-6(7H)-one,
2-allyl-2,4,5,6-tetrahydropyrazolo[3,4-e]indazole-3-carboxylic acid hydrochloride,
1-(benzylthio)-4,5,7,8,9,10-hexahydro[1,4]diazepino[1,2-b]pyrazolo[3,4-g]indazol-6(3H)-one,
2,4,5,6-tetrahydropyrazolo[3,4-e]indazole-3-carboxamide,
8-(methylthio)-2,4,5,6-tetrahydropyrazolo[3,4-e]indazole-3-carboxamide,
1-{1-[(benzyloxy)carbonyl]piperidin-4-yl}-6-trityl-1,4,5,6-tetrahydropyrazolo[3,4-e]indazole-3-carboxylic acid,
ethyl 2-(3-aminopropyl)-8-(methylthio)-2,4,5,6-tetrahydropyrazolo[3,4-e]indazole-3-carboxylate dihydrochloride,
2-piperidin-4-yl-2,4,5,6-tetrahydropyrazolo[3,4-e]indazole-3-carboxylic acid hydrochloride,
1-(2,2,2-trifluoroethyl)-6-trityl-1,4,5,6-tetrahydropyrazolo[3,4-e]indazole-3-carboxylic acid,
1-(2-hydroxyethyl)-1,4,5,6-tetrahydropyrazolo[3,4-e]indazole-3-carboxamide,
[4-(aminocarbonyl)phenyl]-1,4,5,6-tetrahydropyrazolo[3,4-e]indazole-3-carboxamide or (1-[4-(aminocarbonyl)phenyl]-1,4,5,6-tetrahydropyrazolo[3,4-e]indazole-3-carboxamide),
8-(benzylthio)-2,4,5,6-tetrahydropyrazolo[3,4-e]indazole-3-carboxamide,
1-allyl-1,4,5,6-tetrahydropyrazolo[3,4-e]indazole-3-carboxamide,
2-(3-hydroxypropyl)-2,4,5,6-tetrahydropyrazolo[3,4-e]indazole-3-carboxylic acid,
1-(3-aminopropyl)-1,4,5,6-tetrahydropyrazolo[3,4-e]indazole-3-carboxamide dihydrochloride,
1-phenyl-1,4,5,6-tetrahydropyrazolo[3,4-e]indazole-3-carboxamide,
6-(2-hydroxyethyl)-2,4,5,6-tetrahydropyrazolo[3,4-e]indazole-3-carboxylic acid,
2-(3-cyanopropyl)-2,4,5,6-tetrahydropyrazolo[3,4-e]indazole-3-carboxylic acid hydrochloride,
ethyl 2-(3-aminopropyl)-2,4,5,6-tetrahydropyrazolo[3,4-e]indazole-3-carboxylate dihydrochloride,
1-benzyl-1,4,5,6-tetrahydropyrazolo[3,4-e]indazole-3-carboxamide,
1-[4-(methylsulfonyl)phenyl]-1,4,5,6-tetrahydropyrazolo[3,4-e]indazole-3-carboxamide,
ethyl 2-{3-[(2-thien-3-ylethyl)amino]propyl}-2,4,5,6-tetrahydropyrazolo[3,4-e]indazole-3-carboxylate dihydrochloride,
2-(2-hydroxyethyl)-2,4,5,6-tetrahydropyrazolo[3,4-e]indazole-3-carboxylic acid,
2-{2-[(tert-butoxycarbonyl)amino]ethyl}-6-trityl-2,4,5,6-tetrahydropyrazolo[3,4-e]indazole-3-carboxylic acid,
N-methoxy-2,4,5,6-tetrahydropyrazolo[3,4-e]indazole-3-carboxamide hydrochloride,
1-[4-(aminosulfonyl)phenyl]-1,4,5,6-tetrahydropyrazolo[3,4-e]indazole-3-carboxylic acid,
2-(2,2,2-trifluoroethyl)-2,4,5,6-tetrahydropyrazolo[3,4-e]indazole-3-carboxylic acid,
1-(2,2,2-trifluoroethyl)-1,4,5,6-tetrahydropyrazolo[3,4-e]indazole-3-carboxylic acid hydrochloride,
2-allyl-2,4,5,6-tetrahydropyrazolo[3,4-e]indazole-3-carboxamide,
ethyl 2-(3-aminopropyl)-8-(benzylthio)-2,4,5,6-tetrahydropyrazolo[3,4-e]indazole-3-carboxylate dihydrochloride,
2,4,5,6-tetrahydropyrazolo[3,4-e]indazole-3-carboxylic acid hydrochloride,
6-(ethoxymethyl)-2,4,5,6-tetrahydropyrazolo[3,4-e]indazole-3-carboxamide,
ethyl 6-phenyl-2,4,5,6-tetrahydropyrazolo[3,4-e]indazole-3-carboxylate,
1-[4-(aminosulfonyl)phenyl]-6-benzyl-N-methyl-1,4,5,6-tetrahydropyrazolo[3,4-e]indazole-3-carboxamide,
6-methyl-2,4,5,6-tetrahydropyrazolo[3,4-e]indazole-3-carboxamide,
ethyl 6-methyl-2,4,5,6-tetrahydropyrazolo[3,4-e]indazole-3-carboxylate,
6-phenyl-2,4,5,6-tetrahydropyrazolo[3,4-e]indazole-3-carboxamide,
1-[4-(aminosulfonyl)phenyl]-N-methyl-1,4,5,6-tetrahydropyrazolo[3,4-e]indazole-3-carboxamide,
6-benzyl-1-phenyl-1,4,5,6-tetrahydropyrazolo[3,4-e]indazole-3-carboxamide,
ethyl 1-phenyl-1,4,5,6-tetrahydropyrazolo[3,4-e]indazole-3-carboxylate,
ethyl 6-benzyl-1-phenyl-1,4,5,6-tetrahydropyrazolo[3,4-e]indazole-3-carboxylate,
1-(3-amino-3-oxopropyl)-1,4,5,6-tetrahydropyrazolo[3,4-e]indazole-3-carboxamide,
1-(2-amino-2-oxoethyl)-1,4,5,6-tetrahydropyrazolo[3,4-e]indazole-3-carboxamide,
8-(methylsulfonyl)-2,4,5,6-tetrahydropyrazolo[3,4-e]indazole-3-carboxamide,
ethyl 2-{2-[(tert-butoxycarbonyl)amino]ethyl}-6-trityl-2,4,5,6-tetrahydropyrazolo[3,4-e]indazole-3-carboxylate,
8-(benzylthio)-2,4,5,6-tetrahydropyrazolo[3,4-e]indazole-3-carboxylic acid,
ethyl 8-(benzylthio)-2,4,5,6-tetrahydropyrazolo[3,4-e]indazole-3-carboxylate,
8-(methylsulfinyl)-2,4,5,6-tetrahydropyrazolo[3,4-e]indazole-3-carboxylic acid,
ethyl 8-(methylsulfinyl)-2,4,5,6-tetrahydropyrazolo[3,4-e]indazole-3-carboxylate,
8-(methylthio)-2,4,5,6-tetrahydropyrazolo[3,4-e]indazole-3-carboxylic acid,
ethyl 8-(methylthio)-2,4,5,6-tetrahydropyrazolo[3,4-e]indazole-3-carboxylate,
tert-butyl 3-[3-(aminocarbonyl)-5,6-dihydropyrazolo[3,4-e]indazol-2(4H)yl]propylcarbamate,
1-(2-cyanoethyl)-1,4,5,6-tetrahydropyrazolo[3,4-e]indazole-3-carboxamide,
6-[(benzyloxy)methyl]-2,4,5,6-tetrahydropyrazolo[3,4-e]indazole-3-carboxamide,
ethyl 6-(2-hydroxyethyl)-2,4,5,6-tetrahydropyrazolo[3,4-e]indazole-3-carboxylate acetate,
6-(2-hydroxyethyl)-2,4,5,6-tetrahydropyrazolo[3,4-e]indazole-3-carboxamide,
1-phenyl-1,4,5,6-tetrahydropyrazolo[3,4-e]indazole-3-carboxylic acid,
ethyl 2-(2,2,2-trifluoroethyl)-2,4,5,6-tetrahydropyrazolo[3,4-e]indazole-3-carboxylate hydrochloride,
ethyl 1-(2,2,2-trifluoroethyl)-6-trityl-1,4,5,6-tetrahydropyrazolo[3,4-e]indazole-3-carboxylate,
ethyl 2-allyl-6-trityl-2,4,5,6-tetrahydropyrazolo[3,4-e]indazole-3-carboxylate,
ethyl 7-isobutyl-2,4,5,7-tetrahydropyrazolo[3,4-e]indazole-3-carboxylate,
6-(4-methylphenyl)-2,4,5,6-tetrahydropyrazolo[3,4-e]indazole-3-carboxylic acid,
ethyl 6-(4-methylphenyl)-2,4,5,6-tetrahydropyrazolo[3,4-e]indazole-3-carboxylate,
7-[(benzyloxy)methyl]-2,4,5,7-tetrahydropyrazolo[3,4-e]indazole-3-carboxamide,
7-isobutyl-2,4,5,7-tetrahydropyrazolo[3,4-e]indazole-3-carboxamide,
ethyl 1-(2,2,2-trifluoroethyl)-1,4,5,6-tetrahydropyrazolo[3,4-e]indazole-3-carboxylate hydrochloride,
1-[4-(methylsulfonyl)phenyl]-1,4,5,6-tetrahydropyrazolo[3,4-e]indazole-3-carboxylic acid,
ethyl 1-[4-(methylsulfonyl)phenyl]-1,4,5,6-tetrahydropyrazolo[3,4-e]indazole-3-carboxylate,
ethyl 6-benzyl-1-[4-(methylsulfonyl)phenyl]-1,4,5,6-tetrahydropyrazolo[3,4-e]indazole-3-carboxylate,
ethyl 6-[(benzyloxy)methyl]-2,4,5,6-tetrahydropyrazolo[3,4-e]indazole-3-carboxylate,
ethyl 7-[(benzyloxy)methyl]-2,4,5,7-tetrahydropyrazolo[3,4-e]indazole-3-carboxylate,
1-(2,2,2-trifluoroethyl)-1,4,5,6-tetrahydropyrazolo[3,4-e]indazole-3-carboxamide,
ethyl 1-(2-cyanoethyl)-1,4,5,6-tetrahydropyrazolo[3,4-e]indazole-3-carboxylate hydrochloride,
ethyl 1-(3-aminopropyl)-1,4,5,6-tetrahydropyrazolo[3,4-e]indazole-3-carboxylate,
ethyl 1-{3-[(tert-butoxycarbonyl)amino]propyl}-6-trityl-1,4,5,6-tetrahydropyrazolo[3,4-e]indazole-3-carboxylate,
1-(3-aminopropyl)-1,4,5,6-tetrahydropyrazolo[3,4-e]indazole-3-carboxylic acid,
2-(carboxymethyl)-2,4,5,6-tetrahydropyrazolo[3,4-e]indazole-3-carboxylic acid,
ethyl 1-(2-ethoxy-2-oxoethyl)-6-trityl-1,4,5,6-tetrahydropyrazolo[3,4-e]indazole-3-carboxylate,
ethyl 1-(2-cyanoethyl)-6-trityl-1,4,5,6-tetrahydropyrazolo[3,4-e]indazole-3-carboxylate,
2-(3-aminopropyl)-2,4,5,6-tetrahydropyrazolo[3,4-e]indazole-3-carboxamide,
1-allyl-1,4,5,6-tetrahydropyrazolo[3,4-e]indazole-3-carboxylic acid hydrochloride,
6-allyl-2,4,5,6-tetrahydropyrazolo[3,4-e]indazole-3-carboxamide,
7-allyl-2,4,5,7-tetrahydropyrazolo[3,4-e]indazole-3-carboxamide,
ethyl 2-(2-ethoxy-2-oxoethyl)-6-trityl-2,4,5,6-tetrahydropyrazolo[3,4-e]indazole-3-carboxylate,
ethyl 2-(2-hydroxyethyl)-6-trityl-2,4,5,6-tetrahydropyrazolo[3,4-e]indazole-3-carboxylate,
1-(2-carboxyethyl)-1,4,5,6-tetrahydropyrazolo[3,4-e]indazole-3-carboxylic acid,
6-benzyl-1,4,5,6-tetrahydropyrazolo[3,4-e]indazole-3-carboxamide,
ethyl 2-(3-hydroxypropyl)-2,4,5,6-tetrahydropyrazolo[3,4-e]indazole-3-carboxylate hydrochloride,
ethyl 2-[3-(tetrahydro-2H-pyran-2-yloxy)propyl]-6-trityl-2,4,5,6-tetrahydropyrazolo[3,4-e]indazole-3-carboxylate,
[3-(ethoxycarbonyl)-5,6-dihydropyrazolo[3,4-e]indazol-2(4H)-yl]acetic acid,
[3-(methoxycarbonyl)-5,6-dihydropyrazolo[3,4-e]indazol-2(4H)-yl]acetic acid,
1-piperidin-4-yl-1,4,5,6-tetrahydropyrazolo[3,4-e]indazole-3-carboxylic acid dihydrochloride,
1-piperidin-4-yl-6-trityl-1,4,5,6-tetrahydropyrazolo[3,4-e]indazole-3-carboxylic acid hydrochloride,
ethyl 1-{1-[(benzyloxy)carbonyl]piperidin-4-yl}-6-trityl-1,4,5,6-tetrahydropyrazolo[3,4-e]indazole-3-carboxylate,
ethyl 2-(3-cyanopropyl)-6-trityl-2,4,5,6-tetrahydropyrazolo[3,4-e]indazole-3-carboxylate,
ethyl 2-(4-aminobutyl)-2,4,5,6-tetrahydropyrazolo[3,4-e]indazole-3-carboxylate hydrochloride,
1-(3-amino-3-oxopropyl)-1,4,5,6-tetrahydropyrazolo[3,4-e]indazole-3-carboxylic acid,
[3-(ethoxycarbonyl)-5,6-dihydropyrazolo[3,4-e]indazol-[(4H)-yl]acetic acid,
ethyl 2-(2-ethoxy-2-oxoethyl)-2,4,5,6-tetrahydropyrazolo[3,4-e]indazole-3-carboxylate hydrochloride,
1-(2-hydroxyethyl)-1,4,5,6-tetrahydropyrazolo[3,4-e]indazole-3-carboxylic acid,
ethyl 1-(2-hydroxyethyl)-1,4,5,6-tetrahydropyrazolo[3,4-e]indazole-3-carboxylate hydrochloride,
8-(methylsulfonyl)-2,4,5,6-tetrahydropyrazolo[3,4-e]indazole-3-carboxylic acid,
ethyl 8-(methylsulfonyl)-2,4,5,6-tetrahydropyrazolo[3,4-e]indazole-3-carboxylate,
ethyl 6-allyl-2,4,5,6-tetrahydropyrazolo[3,4-e]indazole-3-carboxylate,
ethyl 7-allyl-2,4,5,7-tetrahydropyrazolo[3,4-e]indazole-3-carboxylate,
ethyl 1-(2-hydroxyethyl)-6-trityl-1,4,5,6-tetrahydropyrazolo[3,4-e]indazole-3-carboxylate,
ethyl 1-benzyl-1,4,5,6-tetrahydropyrazolo[3,4-e]indazole-3-carboxylate,
ethyl 2,4,5,6-tetrahydropyrazolo[3,4-e]indazole-3-carboxylate,
ethyl 6-trityl-2,4,5,6-tetrahydropyrazolo[3,4-e]indazole-3-carboxylate,
8-amino-2,4,5,6-tetrahydropyrazolo[3,4-e]indazole-3-carboxylic acid dihydrochloride,
ethyl 2-{3-[(tert-butoxycarbonyl)amino]propyl}-6-[(4-methylphenyl)sulfonyl]-8-(methylthio)-2,4,5,6-tetrahydropyrazolo[3,4-e]indazole-3-carboxylate,
ethyl 8-(tert-butylamino)-2,4,5,6-tetrahydropyrazolo[3,4-e]indazole-3-carboxylate,
8-(tert-butylamino)-2,4,5,6-tetrahydropyrazolo[3,4-e]indazole-3-carboxylic acid hydrochloride,
ethyl 8-(allylthio)-6-[(4-methylphenyl)sulfonyl]-2,4,5,6-tetrahydropyrazolo[3,4-e]indazole-3-carboxylate,
8-Allylsulfanyl-2-(3-tert-butoxycarbonylamino-propyl)-6-(toluene-4-sulfonyl)-2,4,5,6-tetrahydro-1,2,6,7-tetraaza-a s-indacene-3-carboxylic acid ethyl ester

15. A pyridyl pyrazole MK-2 inhibitor that is selected from the group consisting of:
2-(3-aminopropyl)-8-quinolin-3-yl-4,5-dihydro-2H-pyrazolo[3,4-f]isoquinoline-3-carboxylic acid trifluoroacetate,
2-(3-aminopropyl)-8-(3-nitrophenyl)-4,5-dihydro-2H-pyrazolo[3,4-f]isoquinoline-3-carboxylic acid trifluoroacetate,
2-(3-aminopropyl)-8-(4-hydroxyphenyl)-4,5-dihydro-2H-pyrazolo[3,4-f]isoquinoline-3-carboxylic acid hydrobromide,
2-(3-aminopropyl)-8-(3-hydroxyphenyl)-4,5-dihydro-2H-pyrazolo[3,4-f]isoquinoline-3-carboxylic acid hydrobromide,
2-(3-aminopropyl)-8-(2-naphthyl)-4,5-dihydro-2H-pyrazolo[3,4-f]isoquinoline-3-carboxylic acid trifluoroacetate,
2-(3-aminopropyl)-8-(3,5-difluorophenyl)-4,5-dihydro-2H-pyrazolo[3,4-f]isoquinoline-3-carboxylic acid trifluoroacetate,
2-(3-aminopropyl)-8-(1,3-benzodioxol-5-yl)-4,5-dihydro-2H-pyrazolo[3,4-f]isoquinoline-3-carboxylic acid trifluoroacetate,
2-(3-aminopropyl)-8-(3-cyanophenyl)-4,5-dihydro-2H-pyrazolo[3,4-f]isoquinoline-3-carboxylic acid trifluoroacetate,
9-(hydroxymethyl)-2-quinolin-3-yl-5,6,9,10-tetrahydropyrazino[1′,2′:1,5]pyrazolo[3,4-f]isoquinolin-7(8H)-one trifluoroacetate,
2-(3-aminopropyl)-8-(4-methoxyphenyl)-4,5-dihydro-2H-pyrazolo[3,4-f]isoquinoline-3-carboxylic acid trifluoroacetate,
2-(3-aminopropyl)-8-[3-(methylsulfonyl)phenyl]-4,5-dihydro-2H-pyrazolo[3,4-f]isoquinoline-3-carboxylic acid trifluoroacetate,
2-(3-aminopropyl)-8-[3-(trifluoromethyl)phenyl]-4., 5-dihydro-2H-pyrazolo[3,4-f]isoquinoline-3-carboxylic acid trifluoroacetate,
2-(1H-imidazol-1-yl)-5,6,9,10-tetrahydropyrazino[1′,2′:1,5]pyrazolo[3,4-f]isoquinolin-7(8H)-one trifluoroacetate,
2-(3-aminopropyl)-8-(3-methoxyphenyl)-4,5-dihydro-2H-pyrazolo[3,4-f]isoquinoline-3-carboxylic acid trifluoroacetate,
2-(3-aminopropyl)-8-[4-(trifluoromethyl)phenyl]-4,5-dihydro-2H-pyrazolo[3,4-f]isoquinoline-3-carboxylic acid trifluoroacetate,
2-(3-aminopropyl)-8-anilino-4,5-dihydro-2H-pyrazolo[3,4-f]isoquinoline-3-carboxylic acid,
2-(3-aminopropyl)-8-(3,4-difluorophenyl)-4,5-dihydro-2H-pyrazolo[3,4-f]isoquinoline-3-carboxylic acid trifluoroacetate,
2-(3-{[2-(4′-carboxy-1,1′-biphenyl-4-yl)ethyl]amino}propyl)-4,5-dihydro-2H-pyrazolo[3,4-f]isoquinoline-3-carboxylic acid trifluoroacetate,
2-(4-hydroxyphenyl)-5,6,9,10-tetrahydropyrazino[1′,2′:1,5]pyrazolo[3,4-f]isoquinolin-7(8H)-one trifluoroacetate,
2-propyl-8-quinolin-3-yl-4,5-dihydro-2H-pyrazolo[3,4-f]isoquinoline-3-carboxylic acid,
2-[3-({2-[3′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
2-(3-aminopropyl)-8-(4-tert-butylphenyl)-4,5-dihydro-2H-pyrazolo[3,4-f]isoquinoline-3-carboxylic acid trifluoroacetate,
2-[3-({2-[4-(3-furyl)phenyl]ethyl}amino)propyl]-4,5-dihydro-2H-pyrazolo[3,4-f]isoquinoline-3-carboxylic acid trifluoroacetate,
2-(3-{[2-(3′-chloro-1,1′-biphenyl-4-yl)ethyl]amino}propyl)-4,5-dihydro-2H-pyrazolo[3,4-f]isoquinoline-3-carboxylic acid trifluoroacetate,
2-(4-methoxyphenyl)-5,6,8,9,10,11-hexahydro-7H-[1,4]diazepino[1′,2′:1,5]pyrazolo[3,4-f]isoquinolin-7-one,
2-[3-({2-[4′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
2-(3-hydroxyphenyl)-5,6,9,10-tetrahydropyrazino[1′,2′:1,5]pyrazolo[3,4-f]isoquinolin-7(8H)-one,
2-(3-aminopropyl)-N-hydroxy-8-quinolin-3-yl-4,5-dihydro-2H-pyrazolo[3,4-f]isoquinoline-3-carboxamide hydrochloride,
2-[(E)-2-(4-hydroxyphenyl)ethenyl]-5,6,9,10-tetrahydropyrazino[1′,2′:1,5]pyrazolo[3,4-f]isoquinolin-7(8H)-one trifluoroacetate,
2-quinolin-3-yl-5,6,8,9,10,11-hexahydro-7H-[1,4]diazepino[1′,2′:1,5]pyrazolo[3,4-f]isoquinolin-7-one,
2-(4-hydroxyphenyl)-5,6,8,9,10,11-hexahydro-7H-[1,4]diazepino[1′,2′:1,5]pyrazolo[3,4-q]isoquinolin-7-one,
2-(3-{[2-(2′-chloro-1,1′-biphenyl-4-yl)ethyl]amino}propyl)-4,5-dihydro-2H-pyrazolo[3,4-f]isoquinoline-3-carboxylic acid trifluoroacetate,
2-(3-{[2-(4′-tert-butyl-1,1′-biphenyl-4-yl)ethyl]amino}propyl)-4,5-dihydro-2H-pyrazolo[3,4-f]isoquinoline-3-carboxylic acid trifluoroacetate,
2-(3-{[2-(3,4-dichlorophenyl)ethyl]amino}propyl)-4,5-dihydro-2H-pyrazolo[3,4-f]isoquinoline-3-carboxylic acid trifluoroacetate,
2-(3-{[3-(3-chlorophenyl)propyl]amino}propyl)-4,5-dihydro-2H-pyrazolo[3,4-f]isoquinoline-3-carboxylic acid trifluoroacetate,
2-[(E)-2-phenylethenyl]-5,6,9,10-tetrahydropyrazino[1′,2′:1,5]pyrazolo[3,4-f]isoquinolin-7(8H)-one trifluoroacetate,
2-(3-{[2-(4-pyridin-4-ylphenyl)ethyl]amino}propyl)-4,5-dihydro-2H-pyrazolo[3,4-f]isoquinoline-3-carboxylic acid trifluoroacetate,
2-(3-{[3-(4-bromophenyl)propyl]amino}propyl)-4,5-dihydro-2H-pyrazolo[3,4-f]isoquinoline-3-carboxylic acid trifluoroacetate,
2-(3-{[3-(4-tert-butylphenyl)propyl]amino}propyl)-4,5-dihydro-2H-pyrazolo[3,4-f]isoquinoline-3-carboxylic acid trifluoroacetate,
2-(3-{[2-(3′-isopropyl-1,1′-biphenyl-4-yl)ethyl]amino}propyl)-4,5-dihydro-2H-pyrazolo[3,4-f]isoquinoline-3-carboxylic acid trifluoroacetate,
2-{3-[(2-thien-2-ylethyl)amino]propyl}-4,5-dihydro-2H-pyrazolo[3,4-f]isoquinoline-3-carboxylic acid trifluoroacetate,
2-[4-(dimethylamino)phenyl]-5,6,9,10-tetrahydropyrazino[1′,2′:1,5]pyrazolo[3,4-f]isoquinolin-7(8H)-one,
2-(3-{[2-(1,1′-biphenyl-4-yl)ethyl]amino}propyl)-4,5-dihydro-2H-pyrazolo[3,4-f]isoquinoline-3-carboxylic acid trifluoroacetate,
2-(3-methoxyphenyl)-5,6,9,10-tetrahydropyrazino[1′,2′:1,5]pyrazolo[3,4-f]isoquinolin-7(8H)-one,
2-(3-{[2-(4-bromophenyl)ethyl]amino}propyl)-4,5-dihydro-2H-pyrazolo[3,4-f]isoquinoline-3-carboxylic acid trifluoroacetate,
2-(3-{[2-(2,4-dichlorophenyl)ethyl]amino}propyl)-4,5-dihydro-2H-pyrazolo[3,4-f]isoquinoline-3-carboxylic acid trifluoroacetate,
2-{3-[(benzylsulfonyl)amino]propyl}-8-quinolin-3-yl-4,5-dihydro-2H-pyrazolo[3,4-f]isoquinoline-3-carboxylic acid,
9-(aminomethyl)-5,6,9,10-tetrahydropyrazino[1′,2′:1,5]pyrazolo[3,4-f]isoquinolin-7(8H)-one trifluoroacetate,
2-(3-nitrophenyl)-5,6,8,9,10,11-hexahydro-7H-[1,4]diazepino[1′,2′:1,5]pyrazolo[3,4-f]isoquinolin-7-one,
2-(3-aminopropyl)-8-quinolin-3-yl-4,5-dihydro-2H-pyrazolo[3,4-f]isoquinoline-3-carboxamide hydrochloride,
2-(3-{[3-(4-chlorophenyl)propyl]amino}propyl)-4,5-dihydro-2H-pyrazolo[3,4-f]isoquinoline-3-carboxylic acid trifluoroacetate,
2-[3-(dimethylamino)phenyl]-5,6,8,9,10,11-hexahydro-7H [1,4]diazepino[1′,2′:1,5]pyrazolo[3,4-f]isoquinolin-7-one,
2-(3-{[(4-chlorobenzyl)sulfonyl]amino}propyl)-8-quinolin-3-yl-4,5-dihydro-2H-pyrazolo[3,4-f]isoquinoline-3-carboxylic acid,
2-(3-{[2-(4-pyridin-3-ylphenyl)ethyl]amino}propyl)-4,5-dihydro-2H-pyrazolo[3,4-f]isoquinoline-3-carboxylic acid trifluoroacetate,
2-(3-{[2-(4-chlorophenyl)ethyl]amino}propyl)-4,5-dihydro-2H-pyrazolo[3,4-f]isoquinoline-3-carboxylic acid trifluoroacetate,
2-(3-{[2-(5-chlorothien-2-yl)ethyl]amino}propyl)-4,5-dihydro-2H-pyrazolo[3,4-f]isoquinoline-3-carboxylic acid trifluoroacetate,
2-(3-{[3-(4-cyanophenyl)propyl]amino}propyl)-4,5-dihydro-2H-pyrazolo[3,4-f]isoquinoline-3-carboxylic acid trifluoroacetate,
2-(3-{[3-(5-methyl-2-furyl)butyl]amino}propyl)-4,5-dihydro-2H-pyrazolo[3,4-f]isoquinoline-3-carboxylic acid trifluoroacetate,
2-[3-({2-[4-(1-benzothien-3-yl)phenyl]ethyl}amino)propyl]-4,5-dihydro-2H-pyrazolo[3,4-f]isoquinoline-3-carboxylic acid trifluoroacetate,
2-(3-ammoniopropyl)-3-carboxy-4,5-dihydro-2H-pyrazolo[3,4-f]isoquinolin-7-ium dichloride,
2-(4-methoxyphenyl)-5,6,9,10-tetrahydropyrazino[1′,2′:1,5]pyrazolo[3,4-f]isoquinolin-7(8H)-one,
2-(3-{[3-(4-acetylphenyl)propyl]amino}propyl)-4,5-dihydro-2H-pyrazolo[3,4-f]isoquinoline-3-carboxylic acid trifluoroacetate,
2-(3-aminopropyl)-4,5-dihydro-2H-pyrazolo[3,4-f]isoquinoline-3-carboxylic acid trifluoroacetate,
2-[3-({3-[4-(methylsulfonyl)phenyl]propyl}amino)propyl]-4,5-dihydro-2H-pyrazolo[3,4-f]isoquinoline-3-carboxylic acid trifluoroacetate,
2-(3-aminopropyl)-8-(2-methoxyphenyl)-4,5-dihydro-2H-pyrazolo[3,4-f]isoquinoline-3-carboxylic acid trifluoroacetate,
2-[3-({2-[3′-(aminomethyl)-1,1′-biphenyl-4-yl]ethyl}amino)propyl]-4,5-dihydro-2H-pyrazolo[3,4-f]isoquinoline-3-carboxylic acid trifluoroacetate,
2-(2-aminoethyl)-4,5-dihydro-2H-pyrazolo[3,4-f]isoquinoline-3-carboxylic acid dihydrochloride,
2-(3-{[2-(4-nitrophenyl)ethyl]amino}propyl)-4,5-dihydro-2H-pyrazolo[3,4-f]isoquinoline-3-carboxylic acid trifluoroacetate,
2-[3-({2-[2′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
9-(hydroxymethyl)-5,6,9,10-tetrahydropyrazino[1′,2′:1,5]pyrazolo[3,4-f]isoquinolin-7(8H)-one,
2-(3-{[2-(4-methylphenyl)ethyl]amino}propyl)-4,5-dihydro-2H-pyrazolo[3,4-f]isoquinoline-3-carboxylic acid trifluoroacetate,
9-{[(2-thien-2-ylethyl)amino]methyl}-5,6,9,10-tetrahydropyrazino[1′,2′:1,5]pyrazolo[3,4-f]isoquinolin-7(8H)-one,
2-(3-{[2-(4-ethoxyphenyl)ethyl]amino}propyl)-4,5-dihydro-2H-pyrazolo[3,4-f]isoquinoline-3-carboxylic acid trifluoroacetate,
2-(1,3-benzodioxol-5-yl)-5,6,8,9,10,11-hexahydro-7H-[1,4]diazepino[1′,2′:1,5]pyrazolo[3,4-f]isoquinolin-7-one,
2-(3-{[2-(4-methoxyphenyl)ethyl]amino}propyl)-4,5-dihydro-2H-pyrazolo[3,4-f]isoquinoline-3-carboxylic acid trifluoroacetate,
3-[3-(1H-tetraazol-5-yl)-4,5-dihydro-2H-pyrazolo[3,4-f]isoquinolin-2-yl]propan-1-amine hydrochloride,
2-(3-aminopropyl)-8-chloro-4,5-dihydro-2H-pyrazolo[3,4-f]isoquinoline-3-carboxylic acid trifluoroacetate,
2-(3-{[(1R,2S)-2-phenylcyclopropyl]amino}propyl)-4,5-dihydro-2H-pyrazolo[3,4-f]isoquinoline-3-carboxylic acid trifluoroacetate,
2-{3-[(3,3-diphenylpropyl)amino]propyl}-4,5-dihydro-2H-pyrazolo[3,4-f]isoquinoline-3-carboxylic acid trifluoroacetate,
2-(3-{[2-(3-bromo-4-methoxyphenyl)ethyl]amino}propyl)-4,5-dihydro-2H-pyrazolo[3,4-f]isoquinoline-3-carboxylic acid trifluoroacetate,
2-{3-[(4-phenylbutyl)amino]propyl}-4,5-dihydro-2H-pyrazolo[3,4-f]isoquinoline-3-carboxylic acid trifluoroacetate,
2-[3-(2,3-dihydro-1H-inden-2-ylamino)propyl]-4,5-dihydro-2H-pyrazolo[3,4-f]isoquinoline-3-carboxylic acid trifluoroacetate;
2-(2-naphthyl)-5,6,8,9,10,11-hexahydro-7H-[1,4]diazepino[1′,2′:1,5]pyrazolo[3,4-f]isoquinolin-7-one,
2-{3-[(3-phenylpropyl)amino]propyl}-4,5-dihydro-2H-pyrazolo[3,4-f]isoquinoline-3-carboxylic acid trifluoroacetate,
2-(3-{[2-(4-fluorophenyl)ethyl]amino}propyl)-4,5-dihydro-2H-pyrazolo[3,4-f]isoquinoline-3-carboxylic acid trifluoroacetate,
2-{3-[(2-thien-3-ylethyl)amino]propyl}-4,5-dihydro-2H-pyrazolo[3,4-f]isoquinoline-3-carboxylic acid dihydrochloride,
5,6,9,10-tetrahydropyrazino[1′,2′:1,5]pyrazolo[3,4-f]isoquinolin-7(8H)-one,
2-[3-(glycoloylamino)propyl]-8-quinolin-3-yl-4,5-dihydro-2H-pyrazolo[3,4-f]isoquinoline-3-carboxylic acid hydrochloride,
8-quinolin-3-yl-4,5-dihydro-2H-pyrazolo[3,4-f]isoquinoline-3-carboxylic acid,
2-(3-{[2-(4-ethylphenyl)ethyl]amino}propyl)-4,5-dihydro-2H-pyrazolo[3,4-f]isoquinoline-3-carboxylic acid trifluoroacetate,
2-(3-{[2-(2-chlorophenyl)ethyl]amino}propyl)-4,5-dihydro-2H-pyrazolo[3,4-f]isoquinoline-3-carboxylic acid trifluoroacetate,
2-{3-[(2-ethylbutyl)amino]propyl}-4,5-dihydro-2H-pyrazolo[3,4-f]isoquinoline-3-carboxylic acid trifluoroacetate,
8-quinolin-3-yl-4,5-dihydro-2H-pyrazolo[3,4-f]isoquinoline-3-carboxamide,
2-{3-[(2-pyridin-4-ylethyl)amino]propyl}-4,5-dihydro-2H-pyrazolo[3,4-f]isoquinoline-3-carboxylic acid trifluoroacetate,
2-(3-{[2-(4-chlorophenyl)propyl]amino}propyl)-4,5-dihydro-2H-pyrazolo[3,4-f]isoquinoline-3-carboxylic acid trifluoroacetate,
2-(3-{[2-(3-chlorophenyl)ethyl]amino}propyl)-4,5-dihydro-2H-pyrazolo[3,4-f]isoquinoline-3-carboxylic acid trifluoroacetate,
2-[3-(glycoloylamino)propyl]-4,5-dihydro-2H-pyrazolo[3,4-f]isoquinoline-3-carboxylic acid trifluoroacetate,
2-(3-{[2-(3,4-dimethoxyphenyl)ethyl]amino}propyl)-4,5-dihydro-2H pyrazolo[3,4-f]isoquinoline-3-carboxylic acid trifluoroacetate,
2-(1-benzofuran-2-yl)-5,6,8,9,10,11-hexahydro-7H-[1,4]diazepino[1′,2′:1,5]pyrazolo[3,4-f]isoquinolin-7-one,
2-(3-{[4-(2-aminoethyl)phenyl]amino}propyl)-4,5-dihydro-2H-pyrazolo[3,4-f]isoquinoline-3-carboxylic acid trifluoroacetate,
2-(1-naphthyl)-5,6,9,10-tetrahydropyrazino[1′,2′:1,5]pyrazolo[3,4-f]isoquinolin-7(8H)-one,
8-(3-aminopropyl)-5,6,8,9,10,11-hexahydro-7H-[1,4]diazepino[1′,2′:1,5]pyrazolo[3,4-f]isoquinolin-7-one dihydrochloride,
2-anilino-5,6,9,10-tetrahydropyrazino[1′,2′:1,5]pyrazolo[3,4-f]isoquinolin-7(8H)-one,
2-(3-aminopropyl)-8-[2-(trifluoromethyl)phenyl]-4,5-dihydro-2H-pyrazolo[3,4-f]isoquinoline-3-carboxylic acid trifluoroacetate,
9-(azidomethyl)-5,6,9,10-tetrahydropyrazino[1′,2′:1,5]pyrazolo[3,4-f]isoquinolin-7(8H)-one,
9-({[2-(4-chlorophenyl)ethyl]amino}methyl)-5,6,9,10-tetrahydropyrazino[1′,2′:1,5]pyrazolo[3,4-f]isoquinolin-7(8H)-one,
2-phenyl-5,6,8,9,10,11-hexahydro-7H-[1,4]diazepino[1′,2′:1,5]pyrazolo[3,4-f]isoquinolin-7-one,
2-[3-(pentylamino)propyl]-4,5-dihydro-2H-pyrazolo[3,4-f]isoquinoline-3-carboxylic acid trifluoroacetate,
10-(2-aminoethyl)-5,6,9,10-tetrahydropyrazino[1′,2′:1,5]pyrazolo[3,4-f]isoquinolin-7(8H)-one,
2-[3-(allylamino)propyl]-4,5-dihydro-2H-pyrazolo[3,4-f]isoquinoline-3-carboxylic acid trifluoroacetate,
2-(4-aminobutyl)-4,5-dihydro-2H-pyrazolo[3,4-q]isoquinoline-3-carboxylic acid dihydrochloride,
2-(3-{[2-(4-aminophenyl)ethyl]amino}propyl)-4,5-dihydro-2H-pyrazolo[3,4-f]isoquinoline-3-carboxylic acid trifluoroacetate,
2-[(1E)-3,3-dimethylbut-1-enyl]-5,6,9,10-tetrahydropyrazino[1′,2′:1,5]pyrazolo[3,4-f]isoquinolin-7(8H)-one trifluoroacetate,
10-(nitromethyl)-5,6,9,10-tetrahydropyrazino[1′,2′:1,5]pyrazolo[3,4-f]isoquinolin-7(8H)-one,
3-carboxy-2-[3-(methylammonio)propyl]-4,5-dihydro-2H-pyrazolo[3,4-f]isoquinolin-7-ium dichloride,
2-[3-({[(4-butoxyphenyl)amino]carbonyl}amino)propyl]-4,5-dihydro-2H-pyrazolo[3,4-q]isoquinoline-3-carboxylic acid,
2-{3-[(2-pyridin-3-ylethyl)amino]propyl}-4,5-dihydro-2H-pyrazolo[3,4-f]isoquinoline-3-carboxylic acid trifluoroacetate,
2-{3-[(2-pyridin-2-ylethyl)amino]propyl}-4,5-dihydro-2H-pyrazolo[3,4-f]isoquinoline-3-carboxylic acid trifluoroacetate,
2-{3-[(cyclopropylmethyl)amino]propyl}-4,5-dihydro-2H-pyrazolo[3,4-f]isoquinoline-3-carboxylic acid trifluoroacetate,
2-{3-[(2-thien-2-ylpropyl)amino]propyl}-4,5-dihydro-2H-pyrazolo[3,4-q]isoquinoline-3-carboxylic acid trifluoroacetate,
2-methoxy-5,6,9,10-tetrahydropyrazino[1′,2′:1,5]pyrazolo[3,4-f]isoquinolin-7(8H)-one,
2-[3-(dimethylamino)phenyl]-5,6,9,10-tetrahydropyrazino[1′,2′:1,5]pyrazolo[3,4-f]isoquinolin-7(8H)-one,
2-(3-{[2-(1H-pyrrol-1-yl)ethyl]amino}propyl)-4,5-dihydro-2H-pyrazolo[3,4-f]isoquinoline-3-carboxylic acid trifluoroacetate,
2-[3-(benzyloxy)propyl]-8-quinolin-3-yl-4,5-dihydro-2H-pyrazolo[3,4-f]isoquinoline-3-carboxylic acid,
2-{3-[(4-butoxybenzyl)amino]propyl}-4,5-dihydro-2H-pyrazolo[3,4-f]isoquinoline-3-carboxylic acid trifluoroacetate,
2-(1,3-benzodioxol-5-yl)-5,6,9,10-tetrahydropyrazino[1′,2′:1,5]pyrazolo[3,4-q]isoquinolin-7(8H)-one,
2-[(E)-2-(2-fluorophenyl)ethenyl]-5,6,9,10-tetrahydropyrazino[1′,2′:1,5]pyrazolo[3,4-f]isoquinolin-7(8H)-one,
2-[(1E)-hex-1-enyl]-5,6,9,10-tetrahydropyrazino[1′,2′:1,5]pyrazolo[3,4-q]isoquinolin-7(8H)-one trifluoroacetate,
2-anilino-5,6,8,9,10,11-hexahydro-7H-[1,4]diazepino[1′,2′:1,5]pyrazolo[3,4-f]isoquinolin-7-one,
5,6,8,9,10,11-hexahydro-7H-[1,4]diazepino[1′,2′:1,5]pyrazolo[3,4-f]isoquinolin-7-one,
2-chloro-5,6,9,10-tetrahydropyrazino[1′,2′:1,5]pyrazolo[3,4-f]isoquinolin-7(8H)-one,
2-[(E)-2-(4-methoxyphenyl)ethenyl]-5,6,9,10-tetrahydropyrazino[1′,2′:1,5]pyrazolo[3,4-f]isoquinolin-7(8H)-one,
2-(methylthio)-5,6,9,10-tetrahydropyrazino[1′,2′:1,5]pyrazolo[3,4-f]isoquinolin-7(8H)-one,
2-{3-[(2-furylmethyl)amino]propyl}-4,5-dihydro-2H-pyrazolo[3,4-f]isoquinoline-3-carboxylic acid trifluoroacetate,
2-azepan-1-yl-5,6,9,10-tetrahydropyrazino[1′,2′:1,5]pyrazolo[3,4-f]isoquinolin-7(8H)-one trifluoroacetate,
2-(3,6-dihydropyridin-1 (2H)-yl)-5,6,9,10-tetrahydropyrazino[1′,2′:1,5]pyrazolo[3,4-f]isoquinolin-7(8H)-one trifluoroacetate,
9-methyl-5,6,9,10-tetrahydropyrazino[1′,2′:1,5]pyrazolo[3,4-f]isoquinolin-7(8H)-one,
2-(piperidin-3-ylmethyl)-4,5-dihydro-2H-pyrazolo[3,4-f]isoquinoline-3-carboxylic acid hydrochloride,
9-(chloromethyl)-5,6,9,10-tetrahydropyrazino[1′,2′:1,5]pyrazolo[3,4-f]isoquinolin-7(8H)-one,
2-[(4-methoxybenzyl)amino]-5,6,9,10-tetrahydropyrazino[1′,2′:1,5]pyrazolo[3,4-f]isoquinolin-7(8H)-one trifluoroacetate,
2-(3-{[2-(1H-imidazol-4-yl)ethyl]amino}propyl)-4,5-dihydro-2H-pyrazolo[3,4-f]isoquinoline-3-carboxylic acid trifluoroacetate,
2-(benzylamino)-5,6,9,10-tetrahydropyrazino[1′,2′:1,5]pyrazolo[3,4-f]isoquinolin-7(8H)-one trifluoroacetate,
2-(methylthio)-5,6,8,9,10,11-hexahydro-7H-[1,4]diazepino[1′,2′:1,5]pyrazolo[3,4-f]isoquinolin-7-one,
2-{3-[(2-chlorobenzyl)amino]propyl}-4,5-dihydro-2H-pyrazolo[3,4-f]isoquinoline-3-carboxylic acid trifluoroacetate,
2-[3-(benzylamino)propyl-4,5-dihydro-2H-pyrazolo[3,4-f]isoquinoline-3-carboxylic acid trifluoroacetate,
2-[3-({[(4-methoxyphenyl)amino]carbonyl}amino)propyl]-4,5-dihydro-2H-pyrazolo[3,4-f]isoquinoline-3-carboxylic acid,
2-{3-[(2-phenylethyl)amino]propyl}-4,5-dihydro-2H-pyrazolo[3,4-f]isoquinoline-3-carboxylic acid trifluoroacetate,
2-{3-[(thien-2-ylmethyl)amino]propyl}-4,5-dihydro-2H-pyrazolo[3,4-f]isoquinoline-3-carboxylic acid trifluoroacetate,
2-benzyl-5,6,9,10-tetrahydropyrazino[1′,2′:1,5]pyrazolo[3,4-f]isoquinolin-7(8H)-one,
5,6,8,9,10,11-hexahydro-7H-[1,4]diazepino[1′,2′:1,5]pyrazolo[3,4-f]isoquinolin-7-one,
2-{3-[(4-chlorobenzyl)amino]propyl}-4,5-dihydro-2H-pyrazolo[3,4-f]isoquinoline-3-carboxylic acid trifluoroacetate,
2-{3-[(2-phenylpropyl)amino]propyl}-4,5-dihydro-2H-pyrazolo[3,4-f]isoquinoline-3-carboxylic acid trifluoroacetate,
7-oxo-5,6,7,8,9,10-hexahydropyrazino[1′,2′:1,5)pyrazolo[3,4-f]isoquinoline-9-carboxamide trifluoroacetate,
2-(3-hydroxypropyl)-4,5-dihydro-2H-pyrazolo[3,4-f]isoquinoline-3-carboxylic acid,
2-(1,3-dihydro-2H-isoindol-2-yl)-5,6,9,10-tetrahydropyrazino[1′,2′:1,5]pyrazolo[3,4-f]isoquinolin-7(8H)-one trifluoroacetate,
2-{3-[(4-aminophenyl)amino]propyl}-4,5-dihydro-2H-pyrazolo[3,4-f]isoquinoline-3-carboxylic acid trifluoroacetate,
2-(4-hydroxypiperidin-1-yl)-5,6,9,10-tetrahydropyrazino[1′,2′:1,5]pyrazolo[3,4-f]isoquinolin-7(8H)-one trifluoroacetate,
2-{3-[(1H-imidazol-5-ylmethyl)amino]propyl}-4,5-dihydro-2H-pyrazolo[3,4-f]isoquinoline-3-carboxylic acid trifluoroacetate,
2-{3-[(3-chlorobenzyl)amino]propyl}-4,5-dihydro-2H-pyrazolo[3,4-f]isoquinoline-3-carboxylic acid trifluoroacetate,
2-{3-[(pyridin-3-ylcarbonyl)amino]propyl}-4,5-dihydro-2H-pyrazolo[3,4-f]isoquinoline-3-carboxylic acid trifluoroacetate,
4,5-dihydro-2H-pyrazolo[3,4-f]isoquinoline-3-carboxamide,
2-[3-(cyclopentylamino)propyl]-4,5-dihydro-2H-pyrazolo[3,4-f]isoquinoline-3-carboxylic acid dihydrochloride,
2-(3-{[2-(1H-indol-3-yl)ethyl]amino}propyl)-4,5-dihydro-2H-pyrazolo[3,4-f]isoquinoline-3-carboxylic acid,
10-(3-aminopropyl)-5,6,9,10-tetrahydropyrazino[1′,2′:1,5]pyrazolo[3,4-f]isoquinolin-7(8H)-one,
9-(1,2-dihydroxyethyl)-5,6,9,10-tetrahydropyrazino[1′,2′:1,5]pyrazolo[3,4-f]isoquinolin-7(8H)-one,
2-morpholin-4-yl-5,6,9,10-tetrahydropyrazino[1′,2′:1,5]pyrazolo[3,4-f]isoquinolin-7(8H)-one trifluoroacetate,
ethyl 2-(2-aminoethyl)-4,5-dihydro-2H-pyrazolo[3,4-f]isoquinoline-3-carboxylate dihydrochloride,
2-allyl-4,5-dihydro-2H-pyrazolo[3.,4-f]isoquinoline-3-carboxylic acid,
2-quinolin-8-yl-5,6,9,10-tetrahydropyrazino[1′,2′:1,5]pyrazolo[3,4-f]isoquinolin-7(8H)-one,
lithium 2-[3-(2,3-dihydro-1H-inden-2-ylamino)-2-hydroxypropyl]-4,5-dihydro-2H-pyrazolo[3,4-f]isoquinoline-3-carboxylate,
2-(2-hydroxyethyl)-4,5-dihydro-2H-pyrazolo[3,4-f]isoquinoline-3-carboxylic acid,
2-{3-[(2-pyrrolidin-1-ylethyl)amino]propyl}-4,5-dihydro-2H-pyrazolo[3,4-f]isoquinoline-3-carboxylic acid trifluoroacetate,
2-(3,4-dihydroisoquinolin-2(1H)-yl)-5,6,9,10-tetrahydropyrazino[1′,2′:1,5]pyrazolo[3,4-f]isoquinolin-7(8H)-one trifluoroacetate,
2-(3-{[(benzylamino)carbonyl]amino}propyl)-4,5-dihydro-2H-pyrazolo[3,4-f]isoquinoline-3-carboxylic acid,
2-[3-(dimethylamino)propyl]-4,5-dihydro-2H-pyrazolo[3,4-f]isoquinoline-3-carboxylic acid trifluoroacetate,
10-(3-{[2-(4-chlorophenyl)ethyl]amino}propyl)-5,6,9,10-tetrahydropyrazino[1′,2′:1,5]pyrazolo[3,4-f]isoquinolin-7(8H)-one,
2-(3-aminopropyl)-6-chloro-4,5-dihydro-2H-pyrazolo[3,4-f]isoquinoline-3-carboxylic acid trifluoroacetate,
methyl 7-oxo-5,6,7,8,9,10-hexahydropyrazino[1′,2′:1,5]pyrazolo[3,4-f]isoquinoline-9-carboxylate,
2-[2-(glycoloylamino)ethyl]-4,5-dihydro-2H-pyrazolo[3,4-f]isoquinoline-3-carboxylic acid hydrochloride,
2-[2-(isopropylamino)ethyl]-4,5-dihydro-2H-pyrazolo[3,4-f]isoquinoline-3-carboxylic acid hydrochloride,
2-(3-cyanopropyl)-4,5-dihydro-2H-pyrazolo[3,4-f]isoquinoline-3-carboxylic acid,
2-[3-({[(3-cyanophenyl)amino]carbonyl}amino)propyl]-4,5-dihydro-2H-pyrazolo[3,4-f]isoquinoline-3-carboxylic acid,
9-(aminomethyl)-5,6,9,10-tetrahydro-7H-[1,4]oxazino[4′, 3′:1,5]pyrazolo[3,4-f]isoquinolin-7-one trifluoroacetate,
4-methyl-5,6,8,9,10,11-hexahydro-7H-[1,4]diazepino[1′,2′:1,5]pyrazolo[3,4-f]isoquinolin-7-one trifluoroacetate,
2-propyl-4,5-dihydro-2H-pyrazolo[3,4-f]isoquinoline-3-carboxylic acid,
2-(3-bromopropyl)-4,5-dihydro-2H-pyrazolo[3,4-f]isoquinoline-3-carboxylic acid trifluoroacetate,
2-pyrrolidin-1-yl-5,6,9,10-tetrahydropyrazino[1′,2′:1,5]pyrazolo[3,4-f]isoquinolin-7(8H)-one,
2-[3-(isopropylamino)propyl]-4,5-dihydro-2H-pyrazolo[3,4-f]isoquinoline-3-carboxylic acid hydrochloride,
2-(3-amino-3-carboxypropyl)-4,5-dihydro-2H-pyrazolo[3,4-q]isoquinoline-3-carboxylic acid trifluoroacetate,
2-{3-[(aminocarbonyl)amino]propyl}-4,5-dihydro-2H-pyrazolo[3,4-f]isoquinoline-3-carboxylic acid,
9-{[(4-chlorobenzyl)amino]methyl}-5,6,9,10-tetrahydropyrazino[1′,2′:1,5]pyrazolo[3,4-f]isoquinolin-7(8H)-one,
4-chloro-5,6,9,10-tetrahydropyrazino[1′,2′:1,5]pyrazolo[3,4-f]isoquinolin-7(8H)-one,
7-oxo-2-quinolin-3-yl-5,6,7,8,9,10-hexahydropyrazino[1′,2′:1,5]pyrazolo[3,4-f]isoquinoline-9-carboxamide,
2-{3-[(methylsulfonyl)amino]propyl}-4,5-dihydro-2H-pyrazolo[3,4-f]isoquinoline-3-carboxylic acid trifluoroacetate,
lithium 2-{4-[(2-ethylbutyl)amino]butyl}-4,5-dihydro-2H-pyrazolo[3,4-f]isoquinoline-3-carboxylate,
2-(3-{[(4-methoxyphenyl)acetyl]amino}propyl)-4,5-dihydro-2H-pyrazolo[3,4-f]isoquinoline-3-carboxylic acid trifluoroacetate,
7-oxo-6,7,8,9,10,11-hexahydro-5H-[11,4]diazepino[1′,2′:1,5]pyrazolo[3,4-f]isoquinoline-9-carbonitrile,
2-{3-[(isopropylsulfonyl)amino]propyl}-4,5-dihydro-2H-pyrazolo[3,4-f]isoquinoline-3-carboxylic acid trifluoroacetate,
3-[3-(chloromethyl)-4,5-dihydro-2H-pyrazolo[3,4-f]isoquinolin-2-yl]propan-1-amine hydrochloride,
2-(3,3-dimethylbutyl)-5,6,9,10-tetrahydropyrazino[1′,2′:1,5]pyrazolo[3,4-f]isoquinolin-7(8H)-one trifluoroacetate,
2-thiomorpholin-4-yl-5,6,9,10-tetrahydropyrazino[1′,2′:1,5]pyrazolo[3,4-f]isoquinolin-7(8H)-one trifluoroacetate,
ethyl 2-(2-aminoethyl)-8-chloro-4,5-dihydro-2H-pyrazolo[3,4-f]isoquinoline-3-carboxylate,
2-[4-(benzyloxy)phenyl]-5,6,9,10-tetrahydropyrazino[1′,2′:1,5]pyrazolo[3,4-f]isoquinolin-7(8H)-one,
lithium 2-{2-hydroxy-3-[(thien-2-ylmethyl)amino]propyl}-4,5-dihydro-2H-pyrazolo[3,4-f]isoquinoline-3-carboxylate,
2-(3-amino-3-cyanopropyl)-4,5-dihydro-2H-pyrazolo[3,4-f]isoquinoline-3-carboxylic acid trifluoroacetate,
2-[3-(pyridin-3-ylamino)propyl]-4,5-dihydro-2H-pyrazolo[3,4-f]isoquinoline-3-carboxylic acid trifluoroacetate,
2-[3-({[(ethoxycarbonyl)amino]carbonyl}amino)propyl]-4,5-dihydro-2H-pyrazolo[3,4-f]isoquinoline-3-carboxylic acid trifluoroacetate,
2-{3-[4-(aminocarbonyl)piperidin-1-yl]propyl}-4,5-dihydro-2H-pyrazolo[3,4-f]isoquinoline-3-carboxylic acid trifluoroacetate,
2-(4-amino-4-oxobutyl)-4,5-dihydro-2H-pyrazolo[3,4-f]isoquinoline-3-carboxylic acid trifluoroacetate,
8-quinolin-3-yl-4,5-dihydro-2H-pyrazolo[3,4-f]isoquinoline,
2-(3-{[(butylamino)carbonyl]amino}propyl)-4,5-dihydro-2H-pyrazolo[3,4-f]isoquinoline-3-carboxylic acid trifluoroacetate,
3-(2-furyl)-4,5-dihydro-2H-pyrazolo[3,4-f]isoquinoline,
4-anilino-5,6,9,10-tetrahydropyrazino[1′,2′:1,5]pyrazolo[3,4-f]isoquinolin-7(8H)-one,
8-(3-aminopropyl)-5,6,9,10-tetrahydropyrazino[1′,2′:1,5]pyrazolo[3,4-f]isoquinolin-7(8H)-one dihydrochloride,
2-(3-{[(allylamino)carbonyl]amino}propyl)-4,5-dihydro-2H-pyrazolo[3,4-f]isoquinoline-3-carboxylic acid trifluoroacetate,
2-{3-[(2,2,2-trifluoroethyl)amino]propyl}-4,5-dihydro-2H-pyrazolo[3,4-f]isoquinoline-3-carboxylic acid trifluoroacetate,
5,6,9,10-tetrahydropyrido[1′,2′:1,5]pyrazolo[3,4-f]isoquinolin-7(8H)-one,
2-{[2-(dimethylamino)ethyl]amino}-5,6,9,10-tetrahydropyrazino[1′,2′:1,5]pyrazolo[3,4-f]isoquinolin-7(8H)-one trifluoroacetate,
2-{2-[(2-ethylbutyl)amino]ethyl}-4,5-dihydro-2H-pyrazolo[3,4-f]isoquinoline-3-carboxylic acid,
ethyl 2-(2-aminoethyl)-8-ethynyl-4,5-dihydro-2H-pyrazolo[3,4-f]isoquinoline-3-carboxylate hydrochloride,
2-[2-(4-chlorophenyl)ethyl]-5,6,9,10-tetrahydropyrazino[1′,2′:1,5]pyrazolo[3,4-f]isoquinolin-7(8H)-one trifluoroacetate,
4,5-dihydro-2H-pyrazolo[3,4-f]isoquinoline-3-carboxylic acid,
2-piperazin-1-yl-5,6,9,10-tetrahydropyrazino[1′,2′:1,5]pyrazolo[3,4-f]isoquinolin-7(8H)-one trifluoroacetate,
2-{3-[(thien-2-ylacetyl)amino]propyl}-4,5-dihydro-2H-pyrazolo[3,4-f]isoquinoline-3-carboxylic acid trifluoroacetate hydrochloride,
2-[3-(benzoylamino)propyl]-4,5-dihydro-2H-pyrazolo[3,4-f]isoquinoline-3-carboxylic acid trifluoroacetate,
2-[3-(dimethylamino)-2-hydroxypropyl]-N,N-dimethyl-4,5-dihydro-2H-pyrazolo[3,4-f]isoquinoline-3-carboxamide trifluoroacetate,
2-[(2E)-2-(hydroxyimino)ethyl]-4,5-dihydro-2H-pyrazolo[3,4-f]isoquinoline-3-carboxylic acid,
2-allyl-4,5-dihydro-2H-pyrazolo[3,4-f]isoquinoline-3-carboxylic acid 7-oxide,
2-{3-[({[(4-methylphenyl)sulfonyl]amino}carbonyl)amino]propyl}-4,5-dihydro-2H-pyrazolo[3,4-f]isoquinoline-3-carboxylic acid trifluoroacetate,
2-(3-aminopropyl)-6-(methylthio)-4,5-dihydro-2H-pyrazolo[3,4-f]isoquinoline-3-carboxylic acid trifluoroacetate,
2-(3-methylpiperidin-1-yl)-5,6,9,10-tetrahydropyrazino[1′,2′:1,5]pyrazolo[3,4-f]isoquinolin-7(8H)-one trifluoroacetate,
3-(methylthio)-4,5-dihydro-2H-pyrazolo[3,4-f]isoquinoline,
2-(3-aminopropyl)-6-phenyl-4,5-dihydro-2H-pyrazolo[3,4-f]isoquinoline-3-carboxylic acid trifluoroacetate,
lithium 2-{3-[4-(aminocarbonyl)piperidin-1-yl]-2-hydroxypropyl}-4,5-d hydro-2H-pyrazolo[3,4-f]isoquinoline-3-carboxylate,
2-(3-{bis[3-(5-methyl-2-furyl)butyl]amino}propyl)-4,5-dihydro-2H-pyrazolo[3,4-f]isoquinoline-3-carboxylic acid dihydrochloride,
2-[4-(benzyloxy)phenyl]-5,6,8,9,10,11-hexahydro-7H-[1,4]diazepino[1′,2′:1,5]pyrazolo[3,4-f]isoquinolin-7-one,
2-(4-chlorobenzyl)-5,6,9,10-tetrahydropyrazino[1′,2′:1,5]pyrazolo[3,4-f]isoquinolin-7(8H)-one trifluoroacetate,
3-(difluoromethyl)-4,5-dihydro-2H-pyrazolo[3,4-f]isoquinoline,
2-(4-methylpiperazin-1-yl)-5,6,9,10-tetrahydropyrazino[1′,2′:1,5]pyrazolo[3,4-f]isoquinolin-7(8H)-one trifluoroacetate,
2-quinolin-5-yl-5,6,9,10-tetrahydropyrazino[1′,2′:1,5]pyrazolo[3,4-f]isoquinolin-7(8H)-one,
3-[3-(1H-tetraazol-5-yl)-4,5-dihydro-1H-pyrazolo[3,4-f]isoquinolin-1-yl]propan-1-amine hydrochloride,
2-(4-tert-butylphenyl)-5,6,9,10-tetrahydropyrazino[1′,2′:1,5]pyrazolo[3,4-q]isoquinolin-7(8H)-one,
2-[3-(pyridin-4-ylamino)propyl]-4,5-dihydro-2H-pyrazolo[3,4-f]isoquinoline-3-carboxylic acid,
2-[methyl(phenyl)amino]-5,6,9,10-tetrahydropyrazino[1′,2′:1,5]pyrazolo[3,4-f]isoquinolin-7(8H)-one trifluoroacetate,
8-(3-phenylpropyl)-5,6,9,10-tetrahydropyrazino[1′,2′:1,5]pyrazolo[3,4-f]isoquinolin-7(8H)-one trifluoroacetate,
2-propyl-8-quinolin-3-yl-4,5-dihydro-2H-pyrazolo[3,4-f]isoquinoline-3-carboxamide,
2-[3-(isonicotinoylamino)propyl]-4,5-dihydro-2H-pyrazolo[3,4-f]isoquinoline-3-carboxylic acid trifluoroacetate,
2-{3-[(tert-butoxycarbonyl)amino]propyl}-8-chloro-4,5-dihydro-2H-pyrazolo[3,4-f]isoquinoline-3-carboxylic acid,
2-(3-{[(4-butoxyphenyl)sulfonyl]amino}propyl)-4,5-dihydro-2H-pyrazolo[3,4-f]isoquinoline-3-carboxylic acid trifluoroacetate,
2-(3-{[3-(2-furyl)propanoyl]amino}propyl)-4,5-dihydro-2H-pyrazolo[3,4-f]isoquinoline-3-carboxylic acid trifluoroacetate hydrochloride,
2-(4-phenoxyphenyl)-5,6,9,10-tetrahydropyrazino[1′,2′:1,5]pyrazolo[3,4-f]isoquinolin-7(8H)-one,
2-allyl-8-quinolin-3-yl-4,5-dihydro-2H-pyrazolo[3,4-f]isoquinoline-3-carboxamide,
2-(2-cyclohexylethyl)-5,6,9,10-tetrahydropyrazino[1′,2′:1,5]pyrazolo[3,4-f]isoquinolin-7(8H)-one trifluoroacetate,
lithium 2-(2-hydroxy-3-morpholin-4-ylpropyl)-4,5-dihydro-2H-pyrazolo[3,4-f]isoquinoline-3-carboxylate,
2-{3-[(pyridin-2-ylcarbonyl)amino]propyl}-4,5-dihydro-2H-pyrazolo[3,4-f]isoquinoline-3-carboxylic acid trifluoroacetate,
2-(3-{[(4-cyanophenyl)sulfonyl]amino}propyl)-4,5-dihydro-2H-pyrazolo[3,4-f]isoquinoline-3-carboxylic acid trifluoroacetate,
(7E)-5,6,9,10-tetrahydropyrido[1′,2′:1,5]pyrazolo[3,4-f]isoquinolin-7(8H)-one oxime,
2-hexyl-5,6,9,10-tetrahydropyrazino[1′,2′:1,5]pyrazolo[3,4-f]isoquinolin-7(8H)-one trifluoroacetate,
N-methyl-4,5-dihydro-2H-pyrazolo[3,4-f]isoquinoline-3-carboxamide,
2-[3-(4-aminopiperidin-1-yl)-2-hydroxypropyl]-4,5-dihydro-2H-pyrazolo[3,4-f]isoquinoline-3-carboxylic acid bis(trifluoroacetate),
2-(3-{[(3-methoxyphenyl)sulfonyl]amino}propyl)-4,5-dihydro-2H-pyrazolo[3,4-f]isoquinoline-3-carboxylic acid trifluoroacetate,
2-(3-morpholin-4-ylpropyl)-4,5-dihydro-2H-pyrazolo[3,4-q]isoquinoline-3-carboxylic acid,
2-(3-{4-[(tert-butoxycarbonyl)amino]piperidin-1-yl}propyl)-4,5-dihydro-2H-pyrazolo[3,4-f]isoquinoline-3-carboxylic acid trifluoroacetate,
8-methyl-5,6,9,10-tetrahydropyrazino[1′,2′:1,5]pyrazolo[3,4-f]isoquinolin-7(8H)-one,
methyl 2-(2-hydroxyethyl)-4,5-dihydro-2H-pyrazolo[3,4-f]isoquinoline-3-carboxylate,
lithium 2-(2-hydroxy-3-pyrrolidin-1-ylpropyl)-4,5-dihydro-2H-pyrazolo[3,4-f]isoquinoline-3-carboxylate,
lithium 2-{2-hydroxy-3-[(2-thien-2-ylethyl)amino]propyl}-4,5-dihydro-2H-pyrazolo[3,4-f]isoquinoline-3-carboxylate,
2-(3-{[(1-methyl-1H-imidazol-4-yl)sulfonyl]amino}propyl)-4,5-dihydro-2H-pyrazolo[3,4-f]isoquinoline-3-carboxylic acid trifluoroacetate,
2-[3-({[4-(acetylamino)phenyl]sulfonyl}amino)propyl]-4,5-dihydro-2H-pyrazolo[3,4-f]isoquinoline-3-carboxylic acid trifluoroacetate,
[2-(3-aminopropyl)-4,5-dihydro-2H-pyrazolo[3,4-f]isoquinolin-3-yl]methanol hydrochloride,
N-(3-aminopropyl)-4,5-dihydro-2H-pyrazolo[3,4-f]isoquinoline-3-carboxamide dihydrochloride,
2-[3-(2-furoylamino)propyl]-4,5-dihydro-2H-pyrazolo[3,4-f]isoquinoline-3-carboxylic acid trifluoroacetate,
8-[2-(2-chlorophenyl)ethyl]-5,6,8,9,10,11-hexahydro-7H-[1,4]diazepino[1′,2′:1,5]pyrazolo[3,4-f]isoquinolin-7-one trifluoroacetate,
5,6,10,11,12,13-hexahydro-7H,9H-8,12-methano[1,4]diazonino[1′,2′:1,5]pyrazolo[3,4-f]isoquinolin-7-one dihydrochloride,
2-{3-[(1,3-benzodioxol-5-ylcarbonyl)amino]propyl}-4,5-dihydro-2H-pyrazolo[3,4-f]isoquinoline-3-carboxylic acid trifluoroacetate,
2-(2-hydroxy-3-piperazin-1-ylpropyl)-4,5-dihydro-2H-pyrazolo[3,4-f]isoquinoline-3-carboxylic acid dihydrochloride,
2-[3-(7-oxo-5,6,7,8,9,10-hexahydropyrazino[1′,2′:1,5]pyrazolo[3,4-f]isoquinolin-10-yl)propyl]-1H-isoindole-1,3(2H)-dione,
2-(3-{4-[3-(dimethylamino)propyl]piperazin-1-yl}propyl)-4,5-dihydro-2H-pyrazolo[3,4-f]isoquinoline-3-carboxylic acid,
2-[3-(4-methylpiperazin-1-yl)propyl]-4,5-dihydro-2H-pyrazolo[3,4-f]isoquinoline-3-carboxylic acid,
2-(3-piperidin-1-ylpropyl)-4,5-dihydro-2H-pyrazolo[3,4-f]isoquinoline-3-carboxylic acid,
3-[3-(difluoromethyl)-4,5-dihydro-1H-pyrazolo[3,4-f]isoquinolin-1-yl]propan-1-amine hydrochloride,
tert-butyl 3-[3-(difluoromethyl)-4,5-dihydro-1H-pyrazolo[3,4-f]isoquinolin-1-yl]propylcarbamate,
3-[3-(difluoromethyl)-4,5-dihydro-2H-pyrazolo[3,4-f]isoquinolin-2-yl]propan-1-amine hydrochloride,
tert-butyl 3-[3-(difluoromethyl)-4,5-dihydro-2H-pyrazolo[3,4-f]isoquinolin-2-yl]propylcarbamate,
9-(morpholin-4-ylmethyl)-5,6,9,10-tetrahydro-7H-[1,4]oxazino[4′,3′:1,5]pyrazolo[3,4-f]isoquinolin-7-one,
ethyl 2-[1-(aminomethyl)-4-(1,3-dioxo-1,3-dihydro-2H-isoindol-2-yl)butyl]-4,5-dihydro-2H-pyrazolo[3,4-f]isoquinoline-3-carboxylate,
8-(3-phenylpropyl)-5,6,8,9,10,11-hexahydro-7H-[1,4]diazepino[1′,2′-1,5]pyrazolo[3,4-f]isoquinolin-7-one hydrochloride,
ethyl 2-[2-(glycoloylamino)ethyl]-4,5-dihydro-2H-pyrazolo[3,4-f]isoquinoline-3-carboxylate trifluoroacetate,
2-{3-[(tert-butoxycarbonyl)amino]propyl}-6-chloro-4,5-dihydro-2H-pyrazolo[3,4-f]isoquinoline-3-carboxylic acid,
4-chloro-5,6,8,9,10,11-hexahydro-7H-[1,4]diazepino[1′,2′:1,5]pyrazolo[3,4-f]isoquinolin-7-one,
tert-butyl 3-[3-(1H-tetraazol-5-yl)-4,5-dihydro-1H-pyrazolo[3,4-f]isoquinolin-1-yl]propylcarbamate,
tert-butyl 3-[3-(1H-tetraazol-5-yl)-4,5-dihydro-2H-pyrazolo[3,4-f]isoquinolin-2-yl]propylcarbamate,
tert-butyl 3-(3-cyano-4,5-dihydro-2H-pyrazolo[3,4-f]isoquinolin-2-yl)propylcarbamate,
4,5-dihydro-2H-pyrazolo[3,4-f]isoquinoline-3-carbonitrile,
2-[3-(benzyloxy)propyl]-8-quinolin-3-yl-4,5-dihydro-2H-pyrazolo[3,4-f]isoquinoline-3-carboxamide,
3-[3-(aminomethyl)-4,5-dihydro-2H-pyrazolo[3,4-f]isoquinolin-2-yl]propan-1-amine hydrochloride,
ethyl 2-{2-[(tert-butoxycarbonyl)amino]ethyl}-8-ethynyl-4,5-dihydro-2H-pyrazolo[3,4-f]isoquinoline-3-carboxylate,
ethyl 2-{2-[(tert-butoxycarbonyl)amino]ethyl}-8-[(E)-2-(4-methoxyphenyl)ethenyl]-4,5-dihydro-2H-pyrazolo[3,4-f]isoquinoline-3-carboxylate,
ethyl 2-{2-[(tert-butoxycarbonyl)amino]ethyl}-8-[(E)-2-(2-fluorophenyl)ethenyl]-4,5-dihydro-2H-pyrazolo[3,4-f]isoquinoline-3-carboxylate,
ethyl 2-[(2E)-2-(hydroxyimino)ethyl]-4,5-dihydro-2H-pyrazolo[3,4-f]isoquinoline-3-carboxylate,
2-(3-aminopropyl)-4,5-dihydro-2H-pyrazolo[3,4-f]isoquinoline-3-carbonitrile hydrochloride,
2-(2-phenylethyl)-5,6,9,10-tetrahydropyrazino[1′,2′-: 1,5]pyrazolo[3,4-f]isoquinolin-7(8H)-one trifluoroacetate,
{2-[(2-methoxyethoxy)methyl]-4,5-dihydro-2H-pyrazolo[3,4-f]isoquinolin-3-yl}methanol,
ethyl 2-{2-[(tert-butoxycarbonyl)amino]ethyl}-8-[(E)-2-phenylethenyl]-4,5-dihydro-2H-pyrazolo[3,4-f]isoquinoline-3-carboxylate,
ethyl 8-benzyl-2-{2-[(tert-butoxycarbonyl)amino]ethyl}-4,5-dihydro-2H-pyrazolo[3,4-f]isoquinoline-3-carboxylate,
ethyl 2-[(2-methoxyethoxy)methyl]-4,5-dihydro-2H-pyrazolo[3,4-f]isoquinoline-3-carboxylate,
lithium 2-{2-[(tert-butoxycarbonyl)amino]ethyl}-4,5-dihydro-2H-pyrazolo[3,4-f]isoquinoline-3-carboxylate,
ethyl 2-{2-[(tert-butoxycarbonyl)amino]ethyl}-4,5-dihydro-2H-pyrazolo[3,4-f]isoquinoline-3-carboxylate,
methyl 2-methyl-4,5-dihydro-2H-pyrazolo[3,4-f]isoquinoline-3-carboxylate,
methyl 1-methyl-4,5-dihydro-1H-pyrazolo[3,4-f]isoquinoline-3-carboxylate,
2-[3-(propionylamino)propyl]-4,5-dihydro-2H-pyrazolo[3,4-f]isoquinoline-3-carboxylic acid trifluoroacetate,
2-{3-[(cyclohexylcarbonyl)amino]propyl}-4,5-dihydro-2H-pyrazolo[3,4-f]isoquinoline-3-carboxylic acid trifluoroacetate,
2-(3-{[(1-methyl-1H-pyrrol-2-yl)carbonyl]amino}propyl)-4,5-dihydro-2H-pyrazolo[3,4-f]isoquinoline-3-carboxylic acid trifluoroacetate,
ethyl 2-(3-bromopropyl)-4,5-dihydro-2H-pyrazolo[3,4-f]isoquinoline-3-carboxylate,
2-allyl-4,5-dihydro-2H-pyrazolo[3,4-f]isoquinoline-3-carboxamide,
8-(2-oxo-2-thien-3-ylethyl)-5,6,9,10-tetrahydropyrazino[1′,2′:1,5]pyrazolo[3,4-f]isoquinolin-7(8H)-one hydrochloride,
methyl 1-(2-hydroxyethyl)-4,5-dihydro-1H-pyrazolo[3,4-f]isoquinoline-3-carboxylate,
tert-butyl 3-[3-(methylthio)-4,5-dihydro-2H-pyrazolo[3,4-f]isoquinolin-2-yl]propylcarbamate,
3-(4,5-dihydro-2H-pyrazolo[3,4-f]isoquinolin-2-yl)propan-1-amine hydrochloride,
3-[3-(methylthio)-4,5-dihydro-2H-pyrazolo[3,4-f]isoquinolin-2-yl]propan-1-amine hydrochloride,
ethyl 2-(3-aminopropyl)-4,5-dihydro-2H-pyrazolo[3,4-f]isoquinoline-3-carboxylate hydrochloride,
ethyl 2-{3-[(tert-butoxycarbonyl)amino]propyl}-4,5-dihydro-2H-pyrazolo[3,4-f]isoquinoline-3-carboxylate,
tert-butyl 7-oxo-6,7,10,11-tetrahydro-5H-[1,4]diazepino[1′,2′:1,5]pyrazolo[3,4-f]isoquinoline-8(9H)-carboxylate,
ethyl 2-(3-cyanopropyl)-4,5-dihydro-2H-pyrazolo[3,4-f]isoquinoline-3-carboxylate,
3-(methylsulfonyl)-4,5-dihydro-2H-pyrazolo[3,4-f]isoquinoline,
2-{4-[bis(2-ethylbutyl)amino]butyl}-4,5-dihydro-2H-pyrazolo[3,4-f]isoquinoline-3-carboxylic acid hydrochloride,
ethyl 2-{3-[(4-butoxybenzyl)amino]propyl}-4,5-dihydro-2H-pyrazolo[3,4-f]isoquinoline-3-carboxylate,
2-[3-(4-aminopiperidin-1-yl)propyl]-4,5-dihydro-2H-pyrazolo[3,4-f]isoquinoline-3-carboxylic acid trifluoroacetate,
lithium 2-[2-hydroxy-3-(3-oxopiperazin-1-yl)propyl]-4,5-dihydro-2H-pyrazolo[3,4-f]isoquinoline-3-carboxylate,
lithium 2-(2-hydroxy-3-piperidin-1-ylpropyl)-4,5-dihydro-2H-pyrazolo[3,4-f]isoquinoline-3-carboxylate,
2-(3-piperazin-1-ylpropyl)-4,5-dihydro-2H-pyrazolo[3,4-f]isoquinoline-3-carboxylic acid trifluoroacetate,
2-(3-carboxypropyl)-4,5-dihydro-2H-pyrazolo[3,4-f]isoquinoline-3-carboxylic acid trifluoroacetate,
2-(3-pyrrolidin-1-ylpropyl)-4,5-dihydro-2H-pyrazolo[3,4-f]isoquinoline-3-carboxylic acid,
lithium 2-[2-hydroxy-3-(methylamino)propyl]-4,5-dihydro-2H-pyrazolo[3,4-f]isoquinoline-3-carboxylate,
9-{[(4-aminocyclohexyl)amino]methyl}-5,6,9,10-tetrahydro-7H-[1,4]oxazino[4′, 3′:1,5]pyrazolo[3,4-f]isoquinolin-7-one trifluoroacetate,
lithium 2-{3-[(4-aminocyclohexyl)amino]-2-hydroxypropyl}-4,5-dihydro-2H-pyrazolo[3,4-f]isoquinoline-3-carboxylate,
ethyl 2-{3-[(2-thien-2-ylethyl)amino]propyl}-4,5-dihydro-2H-pyrazolo[3,4-f]isoquinoline-3-carboxylate trifluoroacetate,
2-{2-[bis(2-ethylbutyl)amino]ethyl}-4,5-dihydro-2H-pyrazolo[3,4-f]isoquinoline-3-carboxylic acid dihydrochloride,
9-{[(2-thien-2-ylethyl)amino]methyl}-5,6,9,10-tetrahydro-7H-[1,4]oxazino[4′, 3′:1,5]pyrazolo[3,4-f]isoquinolin-7-one trifluoroacetate,
ethyl 2-[3-amino-4-(benzyloxy)-4-oxobutyl]-4,5-dihydro-2H-pyrazolo[3,4-f]isoquinoline-3-carboxylate bis(trifluoroacetate),
ethyl 2-{4-(benzyloxy)-3-[(tert-butoxycarbonyl)amino]-4-oxobutyl}-4,5-dihydro-2H-pyrazolo[3,4-f]isoquinoline-3-carboxylate,
7-oxo-5,6,7,8,9,10-hexahydropyrazino[1′,2′:1,5]pyrazolo[3,4-f]isoquinoline-9-carboxylic acid,
4-(7-oxo-6,7,9,10-tetrahydropyrazino[1′,2′:1,5]pyrazolo[3,4-f]isoquinolin-8(5H)-yl)butanenitrile,
8-(4-aminobutyl)-5,6,9,10-tetrahydropyrazino[1′,2′:1,5]pyrazolo[3,4-f]isoquinolin-7(8H)-one dihydrochloride,
ethyl 6-chloro-4,5-dihydro-2H-pyrazolo[3,4-f]isoquinoline-3-carboxylate,
ethyl 2-{2-[(tert-butoxycarbonyl)amino]ethyl}-8-chloro-4,5-dihydro-2H-pyrazolo[3,4-f]isoquinoline-3-carboxylate,
ethyl 8-chloro-2-{2-[(trifluoroacetyl)amino]ethyl}-4,5-dihydro-2H-pyrazolo[3,4-q]isoquinoline-3-carboxylate,
ethyl 8-chloro-4,5-dihydro-2H-pyrazolo[3,4-f]isoquinoline-3-carboxylate,
8-methyl-5,6,8,9,10,11-hexahydro-7H-[1,4]diazepino[1′,2′:1,5]pyrazolo[3,4-f]isoquinolin-7-one,
ethyl 2-(3-{[3-(5-methyl-2-furyl)butyl]amino}propyl)-4,5-dihydro-2H-pyrazolo[3,4-f]isoquinoline-3-carboxylate trifluoroacetate,
N,2-bis(3-aminopropyl)-4,5-dihydro-2H-pyrazolo[3,4-f]isoquinoline-3-carboxamide dihydrochloride,
2-[3-(benzyloxy)propyl]-4,5-dihydro-2H-pyrazolo[3,4-f]isoquinoline-3-carboxylic acid,
4,5-dihydro-2H-pyrazolo[3,4-f]isoquinolin-3-ylmethanol,
ethyl 2-(2-{[3-(2-furyl)propanoyl]amino}ethyl)-4,5-dihydro-2H-pyrazolo[3,4-f]isoquinoline-3-carboxylate,
2-(2-{[3-(2-furyl)propanoyl]amino}ethyl)-4,5-dihydro-2H-pyrazolo[3,4-f]isoquinoline-3-carboxylic acid hydrochloride,
5,6,7,8,9,10-hexahydropyrazino[1′,2′:1,5]pyrazolo[3,4-f]isoquinolin-9-ylmethanol,
2-chloro-5,6,8,9,10,11-hexahydro-7H-[1,4]diazepino[1′,2′:1,5]pyrazolo[3,4-f]isoquinolin-7-one,
8-{3-[(2-thien-2-ylethyl)amino]propyl}-5,6,8,9,10,11-hexahydro-7H-[1,4]diazepino[1′,2′:1,5]pyrazolo[3,4-f]isoquinolin-7-one dihydrochloride,
9-(aminomethyl)-5,6,8,9,10,11-hexahydro-7H-[1,4]diazepino[1′,2′:1,5]pyrazolo[3,4-f]isoquinolin-7-one,
9-(hydroxymethyl)-8-methyl-5,6,9,10-tetrahydropyrazino[1′,2′-11,5]pyrazolo[3,4-f]isoquinolin-7(8H)-one,
4,5-dihydro-2H-pyrazolo[3,4-f]isoquinoline-3-carboxylic acid,
1-(4,5-dihydro-2H-pyrazolo[3,4-f]isoquinolin-3-yl)-2-methoxyethanone,
ethyl 2-allyl-4,5-dihydro-2H-pyrazolo[3,4-f]isoquinoline-3-carboxylate,
ethyl 1-allyl-4,5-dihydro-1H-pyrazolo[3,4-f]isoquinoline-3-carboxylate,
ethyl 4,5-dihydro-2H-pyrazolo[3,4-f]isoquinoline-3-carboxylate,
ethyl 2-(4-aminobutyl)-4,5-dihydro-2H-pyrazolo[3,4-f]isoquinoline-3-carboxylate,
ethyl 2-(oxiran-2-ylmethyl)-4,5-dihydro-2H-pyrazolo[3,4-f]isoquinoline-3-carboxylate,
2,7-bis(3-cyanopropyl)-3-(ethoxycarbonyl)-4,5-dihydro-2H-pyrazolo[3,4-f]isoquinolin-7-ium,
lithium 2-[2-hydroxy-3-(1H-imidazol-1-yl)propyl]-4,5-dihydro-2H-pyrazolo[3,4-f]isoquinoline-3-carboxylate,
ethyl 2-(3-{[3-(2-furyl)propanoyl]amino}propyl)-4,5-dihydro-2H-pyrazolo[3,4-f]isoquinoline-3-carboxylate,
ethyl 2-{3-[(thien-2-ylacetyl)amino]propyl}-4,5-dihydro-2H-pyrazolo[3,4-f]isoquinoline-3-carboxylate trifluoroacetate,
ethyl 2-[3-(cyclopentylamino)propyl]-4,5-dihydro-2H-pyrazolo[3,4-f]isoquinoline-3-carboxylate,
ethyl 2-(2-aminoethyl)-8-chloro-4,5-dihydro-2H-pyrazolo[3,4-f]isoquinoline-3-carboxylate hydrochloride,
2-(3-{[3-(1,1′-biphenyl-4-yl)propyl]amino}propyl)-4,5-dihydro-2H-pyrazolo[3,4-f]isoquinoline-3-carboxylic acid trifluoroacetate,
2-(3-{[2-(4′-chloro-1,1′-biphenyl-4-yl)ethyl]amino}propyl)-4,5-dihydro-2H-pyrazolo[3,4-f]isoquinoline-3-carboxylic acid trifluoroacetate,
2-(3-{[2-(4-thien-3-ylphenyl)ethyl]amino}propyl)-4,5-dihydro-2H-pyrazolo[3,4-f]isoquinoline-3-carboxylic acid trifluoroacetate,
2-{3-[(2-{4-[(E)-2-phenylethenyl]phenyl}ethyl)amino]propyl}-4,5-dihydro-2H-pyrazolo[3,4-f]isoquinoline-3-carboxylic acid trifluoroacetate,
2-(3-{[2-(4′-cyano-1,1′-biphenyl-4-yl)ethyl]amino}propyl)-4,5-dihydro-2H-pyrazolo[3,4-f]isoquinoline-3-carboxylic acid trifluoroacetate,
2-[3-({2-[4-(1,3-benzodioxol-5-yl)phenyl]ethyl}amino)propyl]-4,5-dihydro-2H-pyrazolo[3,4-f]isoquinoline-3-carboxylic acid trifluoroacetate,
2-quinolin-3-yl-5,6,9,10-tetrahydropyrazino[1′,2′:1,5]pyrazolo[3,4-f]isoquinolin-7(8H)-one,
N-(2-hydroxyethyl)-4,5-dihydro-2H-pyrazolo[3,4-f]isoquinoline-3-carboxamide,
2-(1,1′-biphenyl-4-yl)-5,6,9,10-tetrahydropyrazino[1′,2′:1,5]pyrazolo[3,4-f]isoquinolin-7(8H)-one,
2-[3-({[(4-methoxyphenyl)amino]carbonyl}amino)propyl]-8-quinolin-3-yl-4,5-dihydro-2H-pyrazolo[3,4-f]isoquinoline-3-carboxylic acid,
{2-[(2-methoxyethoxy)methyl]-4,5-dihydro-2H-pyrazolo[3,4-f]isoquinolin-3-yl}acetonitrile,
2-allyl-8-quinolin-3-yl-4,5-dihydro-2H-pyrazolo[3,4-f]isoquinoline-3-carboxylic acid,
2-[(4-methylphenyl)ethynyl]-5,6,9,10-tetrahydropyrazino[1′,2′:1,5]pyrazolo[3,4-f]isoquinolin-7(8H)-one trifluoroacetate,
ethyl 2-(2-aminoethyl)-8-(1H-indol-2-yl)-4,5-dihydro-2H-pyrazolo[3,4-f]isoquinoline-3-carboxylate,
2-[(E)-2-(3-methoxyphenyl)ethenyl]-5,6,9,10-tetrahydropyrazino[1′,2′:1,5]pyrazolo[3,4-f]isoquinolin-7(8H)-one trifluoroacetate,
2-[(E)-2-(3-hydroxyphenyl)ethenyl]-5,6,9,10-tetrahydropyrazino[1′,2′:1,5]pyrazolo[3,4-f]isoquinolin-7(8H)-one trifluoroacetate,
12593 or 7-oxo-2-quinolin-3-yl-5,6,7,8,9,10-hexahydropyrazino[1′,2′:1,5]pyrazolo[3,4-f]isoquinoline-9-carbonitrile trifluoroacetate.

16. A pyrimidyl pyrazole MK-2 inhibitor that is selected from the group consisting of:
2-(3-aminopropyl)-8-quinolin-3-yl-4,5-dihydro-2H-pyrazolo[4,3-h]quinazoline-3-carboxylic acid dihydrochloride,
2-(3-aminopropyl)-8-(1,3-benzodioxol-5-yl)-4,5-dihydro-2H-pyrazolo[4,3-h]quinazoline-3-carboxylic acid hydrochloride,
2-(3-aminopropyl)-8-phenyl-4,5-dihydro-2H-pyrazolo[4,3-h]quinazoline-3-carboxylic acid hydrochloride,
2-quinolin-3-yl-5,6,8,9,10,11-hexahydro-7H-[1,4]diazepino[1′,2′:1,5]pyrazolo[4,3-h]quinazolin-7-one,
2-pyridin-3-yl-5,6,8,9,10,11-hexahydro-7H-[1,4]diazepino[1′,2′:1,5]pyrazolo[4,3-h]quinazolin-7-one,
8-quinolin-3-yl-2-[3-(tritylamino)propyl]-4,5-dihydro-2H-pyrazolo[4,3-h]quinazoline-3-carboxylic acid hydrochloride,
2-(1,3-benzodioxol-5-yl)-5,6,8,9,10,11-hexahydro-7H-[1,4]diazepino[1′,2′:1,5]pyrazolo[4,3-h]quinazolin-7-one,
2-(4-methoxyphenyl)-5,6,8,9,10,11-hexahydro-7H-[1,4]diazepino[1′,2′:1,5]pyrazolo[4,3-h]quinazolin-7-one,
2-pyridin-4-yl-5,6,8,9,10,11-hexahydro-7H-[1,4]diazepino[1′,2′:1,5]pyrazolo[4,3-h]quinazolin-7-one hydrochloride,
8-phenyl-2-[3-(tritylamino)propyl]-4,5-dihydro-2H-pyrazolo[4,3-h]quinazoline-3-carboxylic acid,
2-phenyl-5,6,8,9,10,11-hexahydro-7H-[1,4]diazepino[1′,2′:1,5]pyrazolo[4,3-h]quinazolin-7-one,
methyl 2-(3-aminopropyl)-8-pyridin-4-yl-4,5-dihydro-2H-pyrazolo[4,3-h]quinazoline-3-carboxylate dihydrochloride,
methyl 8-phenyl-2-[3-(tritylamino)propyl]-4,5-dihydro-2H-pyrazolo[4,3-h]quinazoline-3-carboxylate,
methyl 2-(3-aminopropyl)-8-(4-methoxyphenyl)-4,5-dihydro-2H-pyrazolo[4,3-h]quinazoline-3-carboxylate dihydrochloride,
ethyl 2-(3-aminopropyl)-8-quinolin-3-yl-4,5-dihydro-2H-pyrazolo[4,3-h]quinazoline-3-carboxylate dihydrochloride

17. The cyclic pyrazole MK-2 inhibitor according to claim 1, wherein the MK-2 inhibitor has an MK-2 IC₅₀ of less than 100 and is selected from the group consisting of:
2-(3-aminopropyl)-8-(methylthio)-2,4,5,6-tetrahydropyrazolo[3,4-e]indazole-3-carboxylic acid dihydrochloride,
2-(3-aminopropyl)-2,4,5,6-tetrahydropyrazolo[3,4-e]indazole-3-carboxylic acid hydrochloride,
2-(3-{[2-(4-bromophenyl)ethyl]amino}propyl)-2,4,5,6-tetrahydropyrazolo[3,4-e]indazole-3-carboxylic acid hydrochloride,
2-(2-aminoethyl)-2,4,5,6-tetrahydropyrazolo[3,4-e]indazole-3-carboxylic acid hydrochloride,
8-(allylthio)-2-(3-aminopropyl)-2,4,5,6-tetrahydropyrazolo[3,4-e]indazole-3-carboxylic acid dihydrochloride,
2-(3-aminopropyl)-8-(benzylthio)-2,4,5,6-tetrahydropyrazolo[3,4-e]indazole-3-carboxylic acid dihydrochloride,
2-{3-[(2-thien-2-ylethyl)amino]propyl}-2,4,5,6-tetrahydropyrazolo[3,4-e]indazole-3-carboxylic acid,
2-{3-[(2-thien-3-ylethyl)amino]propyl}-2,4,5,6-tetrahydropyrazolo[3,4-e]indazole-3-carboxylic acid hydrochloride,
ethyl 2-(3-{[2-(4-bromophenyl)ethyl]amino}propyl)-2,4,5,6-tetrahydropyrazolo[3,4-e]indazole-3-carboxylate,
2-(4-aminobutyl)-2,4,5,6-tetrahydropyrazolo[3,4-e]indazole-3-carboxylic acid dihydrochloride,
1-(methylthio)-4,5,7,8,9,10-hexahydro[1,4]diazepino[1,2-b]pyrazolo[3.,4-g]indazol-6(3H)-one,
1-(allylthio)-4,5,7,8,9,10-hexahydro[1,4]diazepino[1,2-b]pyrazolo[3,4-g]indazol-6(3H)-one,
4,5,7,8,9,10-hexahydro[1,4]diazepino[1,2-b]pyrazolo[3,4-g]indazol-6(3H)-one 4,5,8,9-tetrahydro-3H-pyrazino[1,2-b]pyrazolo[3,4-g]indazol-6(7H)-one,
2-allyl-2,4,5,6-tetrahydropyrazolo[3,4-e]indazole-3-carboxylic acid hydrochloride,
1-(benzylthio)-4,5,7,8,9,10-hexahydro[1,4]diazepino[1,2-b]pyrazolo[3,4-g]indazol-6(3H)-one,
2,4,5,6-tetrahydropyrazolo[3,4-e]indazole-3-carboxamide,
8-(methylthio)-2,4,5,6-tetrahydropyrazolo[3,4-e]indazole-3-carboxamide,
1-{1-[(benzyloxy)carbonyl]piperidin-4-yl}-6-trityl-1,4,5,6-tetrahydropyrazolo[3,4-e]indazole-3-carboxylic acid,
ethyl 2-(3-aminopropyl)-8-(methylthio)-2,4,5,6-tetrahydropyrazolo[3,4-e]indazole-3-carboxylate dihydrochloride,
2-piperidin-4-yl-2,4,5,6-tetrahydropyrazolo[3,4-e]indazole-3-carboxylic acid hydrochloride,
1-(2,2,2-trifluoroethyl)-6-trityl-1,4,5,6-tetrahydropyrazolo[3,4-e]indazole-3-carboxylic acid,
1-(2-hydroxyethyl)-1,4,5,6-tetrahydropyrazolo[3,4-e]indazole-3-carboxamide,
1-[4-(aminocarbonyl)phenyl]-1,4,5,6-tetrahydropyrazolo[3,4-e]indazole-3-carboxamide (or 1-[4-(aminocarbonyl)phenyl]-1,4,5,6-tetrahydropyrazolo[3,4-e]indazole-3-carboxamide),
8-(benzylthio)-2,4,5,6-tetrahydropyrazolo[3,4-e]indazole-3-carboxamide,
1-allyl-1,4,5,6-tetrahydropyrazolo[3,4-e]indazole-3-carboxamide,
2-(3-aminopropyl)-8-quinolin-3-yl-4,5-dihydro-2H-pyrazolo[4,3-h]quinazoline-3-carboxylic acid dihydrochloride,
2-(3-aminopropyl)-8-(1,3-benzodioxol-5-yl)-4,5-dihydro-2H-pyrazolo[4,3-h]quinazoline-3-carboxylic acid hydrochloride,
2-(3-aminopropyl)-8-phenyl-4,5-dihydro-2H-pyrazolo[4,3-h]quinazoline-3-carboxylic acid hydrochloride,
2-quinolin-3-yl-5,6,8,9,10,11-hexahydro-7H-[1,4]diazepino[1′,2′:1,5]pyrazolo[4,3-h]quinazolin-7-one,
2-pyridin-3-yl-5,6,8,9,10,11-hexahydro-7H-[1,4]diazepino[1′,2′:1,5]pyrazolo[4,3-h]quinazolin-7-one,
8-quinolin-3-yl-2-[3-(tritylamino)propyl]-4,5-dihydro-2H-pyrazolo[4,3-h]quinazoline-3-carboxylic acid hydrochloride,
2-(1,3-benzodioxol-5-yl)-5,6,8,9,10,11-hexahydro-7H-[1,4]diazepino[1′,2′:1,5]pyrazolo[4,3-h]quinazolin-7-one,
2-(4-methoxyphenyl)-5,6,8,9,10,11-hexahydro-7H-[1,4]diazepino[1′,2′:1,5]pyrazolo[4,3-h]quinazolin-7-one,
2-pyridin-4-yl-5,6,8,9,10,11-hexahydro-7H-[1,4]diazepino[1′,2′:1,5]pyrazolo[4,3-h]quinazolin-7-one hydrochloride,
8-phenyl-2-[3-(tritylamino)propyl]-4,5-dihydro-2H-pyrazolo[4,3-h]quinazoline-3-carboxylic acid,
2-phenyl-5,6,8,9,10,11-hexahydro-7H-[1,4]diazepino[1′,2′:1,5]pyrazolo[4,3-h]quinazolin-7-one,
2-(3-aminopropyl)-8-quinolin-3-yl-4,5-dihydro-2H-pyrazolo[3,4-f]isoquinoline-3-carboxylic acid trifluoroacetate,
2-(3-aminopropyl)-8-(3-nitrophenyl)-4,5-dihydro-2H-pyrazolo[3,4-f]isoquinoline-3-carboxylic acid trifluoroacetate,
2-(3-aminopropyl)-8-(4-hydroxyphenyl)-4,5-dihydro-2H-pyrazolo[3,4-f]isoquinoline-3-carboxylic acid hydrobromide,
2-(3-aminopropyl)-8-(3-hydroxyphenyl)-4,5-dihydro-2H-pyrazolo[3,4-f]isoquinoline-3-carboxylic acid hydrobromide,
2-(3-aminopropyl)-8-(2-naphthyl)-4,5-dihydro-2H-pyrazolo[3,4-f]isoquinoline-3-carboxylic acid trifluoroacetate,
2-(3-aminopropyl)-8-(3,5-difluorophenyl)-4,5-dihydro-2H-pyrazolo[3,4-f]isoquinoline-3-carboxylic acid trifluoroacetate,
2-(3-aminopropyl)-8-(1,3-benzodioxol-5-yl)-4,5-dihydro-2H-pyrazolo[3,4-f]isoquinoline-3-carboxylic acid trifluoroacetate,
2-(3-aminopropyl)-8-(3-cyanophenyl)-4,5-dihydro-2H-pyrazolo[3,4-f]isoquinoline-3-carboxylic acid trifluoroacetate,
9-(hydroxymethyl)-2-quinolin-3-yl-5,6,9,10-tetrahydropyrazino[1′,2′:1,5]pyrazolo[3,4-f]isoquinolin-7(8H)-one trifluoroacetate,
2-(3-aminopropyl)-8-(4-methoxyphenyl)-4,5-dihydro-2H-pyrazolo[3,4-f]isoquinoline-3-carboxylic acid trifluoroacetate,
2-(3-aminopropyl)-8-[3-(methylsulfonyl)phenyl]-4,5-dihydro-2H-pyrazolo[3,4-f]isoquinoline-3-carboxylic acid trifluoroacetate,
2-(3-aminopropyl)-8-[3-(trifluoromethyl)phenyl]-4,5-dihydro-2H-pyrazolo[3,4-f]isoquinoline-3-carboxylic acid trifluoroacetate,
2-(1H-imidazol-1-yl)-5,6,9,10-tetrahydropyrazino[1′,2′:1,5]pyrazolo[3,4-f]isoquinolin-7(8H)-one trifluoroacetate,
2-(3-aminopropyl)-8-(3-methoxyphenyl)-4,5-dihydro-2H-pyrazolo[3,4-f]isoquinoline-3-carboxylic acid trifluoroacetate,
2-(3-aminopropyl)-8-[4-(trifluoromethyl)phenyl]-4,5-dihydro-2H-pyrazolo[3,4-f]isoquinoline-3-carboxylic acid trifluoroacetate,
2-(3-aminopropyl)-8-anilino-4,5-dihydro-2H-pyrazolo[3,4-f]isoquinoline-3-carboxylic acid,
2-(3-aminopropyl)-8-(3,4-difluorophenyl)-4,5-dihydro-2H-pyrazolo[3,4-f]isoquinoline-3-carboxylic acid trifluoroacetate,
2-(3-{[2-(4′-carboxy-1,1′-biphenyl-4-yl)ethyl]amino}propyl)-4,5-dihydro-2H-pyrazolo[3,4-f]isoquinoline-3-carboxylic acid trifluoroacetate,
2-(4-hydroxyphenyl)-5,6,9,10-tetrahydropyrazino[1′,2′:1,5]pyrazolo[3,4-f]isoquinolin-7(8H)-one trifluoroacetate,
2-propyl-8-quinolin-3-yl-4,5-dihydro-2H-pyrazolo[3,4-f]isoquinoline-3-carboxylic acid,
2-[3-({2-[3′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
2-(3-aminopropyl)-8-(4-tert-butylphenyl)-4,5-dihydro-2H-pyrazolo[3,4-f]isoquinoline-3-carboxylic acid trifluoroacetate,
2-[3-({2-[4-(3-furyl)phenyl]ethyl}amino)propyl]-4,5-dihydro-2H-pyrazolo[3,4-f]isoquinoline-3-carboxylic acid trifluoroacetate,
2-(3-{[2-(3′-chloro-1,1′-biphenyl-4-yl)ethyl]amino}propyl)-4,5-dihydro-2H-pyrazolo[3,4-f]isoquinoline-3-carboxylic acid trifluoroacetate,
2-(4-methoxyphenyl)-5,6,8,9,10,11-hexahydro-7H-[1,4]diazepino[1′,2′:1,5]pyrazolo[3,4-f]isoquinolin-7-one,
2-[3-({2-[4′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
2-(3-hydroxyphenyl)-5,6,9,10-tetrahydropyrazino[1′,2′:1,5]pyrazolo[3,4-f]isoquinolin-7(8H)-one,
2-(3-aminopropyl)-N-hydroxy-8-quinolin-3-yl-4,5-dihydro-2H-pyrazolo[3,4-f]isoquinoline-3-carboxamide hydrochloride,
2-[(E)-2-(4-hydroxyphenyl)ethenyl]-5,6,9,10-tetrahydropyrazino[1′,2′:1,5]pyrazolo[3,4-f]isoquinolin-7(8H)-one trifluoroacetate,
2-quinolin-3-yl-5,6,8,9,10,11-hexahydro-7H-[1,4]diazepino[1′,2′:1,5]pyrazolo[3,4-f]isoquinolin-7-one,
2-(4-hydroxyphenyl)-5,6,8,9,10,11-hexahydro-7H-[1,4]diazepino[1′,2′:1,5]pyrazolo[3,4-f]isoquinolin-7-one,
2-(3-{[2-(2′-chloro-1,1′-biphenyl-4-yl)ethyl]amino}propyl)-4,5-dihydro-2H-pyrazolo[3,4-f]isoquinoline-3-carboxylic acid trifluoroacetate,
2-(3-{[2-(4′-tert-butyl-1,1′-biphenyl-4-yl)ethyl]amino}propyl)-4,5-dihydro-2H-pyrazolo[3,4-f]isoquinoline-3-carboxylic acid trifluoroacetate,
2-(3-{[2-(3,4-dichlorophenyl)ethyl]amino}propyl)-4,5-dihydro-2H-pyrazolo[3,4-f]isoquinoline-3-carboxylic acid trifluoroacetate,
2-(3-{[3-(3-chlorophenyl)propyl]amino}propyl)-4,5-dihydro-2H-pyrazolo[3,4-f]isoquinoline-3-carboxylic acid trifluoroacetate,
2-[(E)-2-phenylethenyl]-5,6,9,10-tetrahydropyrazino[1′,2′:1,5]pyrazolo[3,4-f]isoquinolin-7(8H)-one trifluoroacetate,
2-(3-{[2-(4-pyridin-4-ylphenyl)ethyl]amino}propyl)-4,5-dihydro-2H-pyrazolo[3,4-f]isoquinoline-3-carboxylic acid trifluoroacetate,
2-(3-{[3-(4-bromophenyl)propyl]amino}propyl)-4,5-dihydro-2H-pyrazolo[3,4-f]isoquinoline-3-carboxylic acid trifluoroacetate,
2-(3-{[3-(4-tert-butylphenyl)propyl]amino}propyl)-4,5-dihydro-2H-pyrazolo[3,4-f]isoquinoline-3-carboxylic acid trifluoroacetate,
2-(3-{[2-(3′-isopropyl-1,1′-biphenyl-4-yl)ethyl]amino}propyl)-4,5-dihydro-2H-pyrazolo[3,4-f]isoquinoline-3-carboxylic acid trifluoroacetate,
2-{3-[(2-thien-2-ylethyl)amino]propyl}-4,5-dihydro-2H-pyrazolo[3,4-f]isoquinoline-3-carboxylic acid trifluoroacetate,
2-[4-(dimethylamino)phenyl]-5,6,9,10-tetrahydropyrazino[1′,2′:1,5]pyrazolo[3,4-f]isoquinolin-7(8H)-one,
2-(3-{[2-(1,1′-biphenyl-4-yl)ethyl]amino}propyl)-4,5-dihydro-2H-pyrazolo[3,4-f]isoquinoline-3-carboxylic acid trifluoroacetate,
2-(3-methoxyphenyl)-5,6,9,10-tetrahydropyrazino[1′,2′:1,5]pyrazolo[3,4-f]isoquinolin-7(8H)-one,
2-(3-{[2-(4-bromophenyl)ethyl]amino}propyl)-4,5-dihydro-2H-pyrazolo[3,4-f]isoquinoline-3-carboxylic acid trifluoroacetate,
2-(3-{[2-(2,4-dichlorophenyl)ethyl]amino}propyl)-4,5-dihydro-2H-pyrazolo[3,4-q]isoquinoline-3-carboxylic acid trifluoroacetate,
2-{3-[(benzylsulfonyl)amino]propyl}-8-quinolin-3-yl-4,5-dihydro-2H--pyrazolo[3,4-f]isoquinoline-3-carboxylic acid,
9-(aminomethyl)-5,6,9,10-tetrahydropyrazino[1′,2′:1,5]pyrazolo[3,4-f]isoquinolin-7(8H)-one trifluoroacetate,
2-(3-nitrophenyl)-5,6,8,9,10,11-hexahydro-7H-[1,4]diazepino[1′,2′:1,5]pyrazolo[3,4-f]isoquinolin-7-one,
2-(3-aminopropyl)-8-quinolin-3-yl-4,5-dihydro-2H-pyrazolo[3,4-f]isoquinoline-3-carboxamide hydrochloride,
2-(3-{[3-(4-chlorophenyl)propyl]amino}propyl)-4,5-dihydro-2H-pyrazolo[3,4-f]isoquinoline-3-carboxylic acid trifluoroacetate,
2-[3-(dimethylamino)phenyl]-5,6,8,9,10,11-hexahydro-7H-[1,4]diazepino[1′,2′:1,5]pyrazolo[3,4-f]isoquinolin-7-one,
2-(3-{[(4-chlorobenzyl)sulfonyl]amino}propyl)-8-quinolin-3-yl-4,5-dihydro-2H-pyrazolo[3,4-f]isoquinoline-3-carboxylic acid,
2-(3-{[2-(4-pyridin-3-ylphenyl)ethyl]amino}propyl)-4,5-dihydro-2H-pyrazolo[3,4-f]isoquinoline-3-carboxylic acid trifluoroacetate,
2-(3-{[2-(4-chlorophenyl)ethyl]amino}propyl)-4,5-dihydro-2H-pyrazolo[3,4-f]isoquinoline-3-carboxylic acid trifluoroacetate,
2-(3-{[2-(5-chlorothien-2-yl)ethyl]amino}propyl)-4,5-dihydro-2H-pyrazolo[3,4-f]isoquinoline-3-carboxylic acid trifluoroacetate,
2-(3-{[3-(4-cyanophenylypropyl]amino}propyl)-4,5-dihydro-2H-pyrazolo[3,4-f]isoquinoline-3-carboxylic acid trifluoroacetate,
2-(3-{[3-(5-methyl-2-furyl)butyl]amino}propyl)-4,5-dihydro-2H-pyrazolo[3,4-f]isoquinoline-3-carboxylic acid trifluoroacetate,
2-[3-({2-[4-(1-benzothien-3-yl)phenyl]ethyl}amino)propyl]-4,5-dihydro-2H-pyrazolo[3,4-f]isoquinoline-3-carboxylic acid trifluoroacetate,
2-(3-ammoniopropyl)-3-carboxy-4,5-dihydro-2H-pyrazolo[3,4-f]isoquinolin-7-ium dichloride,
2-(4-methoxyphenyl)-5,6,9,10-tetrahydropyrazino[1′,2′:1,5]pyrazolo[3,4-f]isoquinolin-7(8H)-one,
2-(3-{[3-(4-acetylphenyl)propyl]amino}propyl)-4,5-dihydro-2H-pyrazolo[3,4-f]isoquinoline-3-carboxylic acid trifluoroacetate,
2-(3-aminopropyl)-4,5-dihydro-2H-pyrazolo[3,4-f]isoquinoline-3-carboxylic acid trifluoroacetate,
2-[3-({3-[4-(methylsulfonyl)phenyl]propyl}amino)propyl]-4,5-dihydro-2H-pyrazolo[3,4-f]isoquinoline-3-carboxylic acid trifluoroacetate,
2-(3-aminopropyl)-8-(2-methoxyphenyl)-4,5-dihydro-2H-pyrazolo[3,4-f]isoquinoline-3-carboxylic acid trifluoroacetate,
2-[3-({2-[3′-(aminomethyl)-1,1′-biphenyl-4-yl]ethyl}amino)propyl]-4,5-dihydro-2H-pyrazolo[3,4-f]isoquinoline-3-carboxylic acid trifluoroacetate,
2-(2-aminoethyl)-4,5-dihydro-2H-pyrazolo[3,4-f]isoquinoline-3-carboxylic acid dihydrochloride,
2-(3-{[2-(4-nitrophenyl)ethyl]amino}propyl)-4,5-dihydro-2H-pyrazolo[3,4-f]isoquinoline-3-carboxylic acid trifluoroacetate,
2-[3-({2-[2′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
9-(hydroxymethyl)-5,6,9,10-tetrahydropyrazino[1′,2′:1,5]pyrazolo[3,4-f]isoquinolin-7(8H)-one,
2-(3-{[2-(4-methylphenyl)ethyl]amino}propyl)-4,5-dihydro-2H-pyrazolo[3,4-f]isoquinoline-3-carboxylic acid trifluoroacetate,
9-{[(2-thien-2-ylethyl)amino]methyl}-5,6,9,10-tetrahydropyrazino[1′,2′:1,5]pyrazolo[3,4-f]isoquinolin-7(8H)-one,
2-(3-{[2-(4-ethoxyphenyl)ethyl]amino}propyl)-4,5-dihydro-2H-pyrazolo[3,4-f]isoquinoline-3-carboxylic acid trifluoroacetate,
2-(1,3-benzodioxol-5-yl)-5,6,8,9,10,11-hexahydro-7H-[1,4]diazepino[1′,2′-: 1,5]pyrazolo[3,4-f]isoquinolin-7-one,
2-(3-{[2-(4-methoxyphenyl)ethyl]amino}propyl)-4,5-dihydro-2H-pyrazolo[3,4-f]isoquinoline-3-carboxylic acid trifluoroacetate,
3-[3-(1H-tetraazol-5-yl)-4,5-dihydro-2H-pyrazolo[3,4-f]isoquinolin-2-yl]propan-1-amine hydrochloride,
2-(3-aminopropyl)-8-chloro-4,5-dihydro-2H-pyrazolo[3,4-f]isoquinoline-3-carboxylic acid trifluoroacetate,
2-(3-{[(1R,2S)-2-phenylcyclopropyl]amino}propyl)-4,5-dihydro-2H-pyrazolo[3,4-f]isoquinoline-3-carboxylic acid trifluoroacetate,
2-{3-[(3,3-diphenylpropyl)amino]propyl}-4,5-dihydro-2H-pyrazolo[3,4-f]isoquinoline-3-carboxylic acid trifluoroacetate,
2-(3-{[2-(3-bromo-4-methoxyphenyl)ethyl]amino}propyl)-4,5-dihydro-2H-pyrazolo[3,4-f]isoquinoline-3-carboxylic acid trifluoroacetate,
2-{3-[(4-phenylbutyl)amino]propyl}-4,5-dihydro-2H-pyrazolo[3,4-f]isoquinoline-3-carboxylic acid trifluoroacetate,
2-[3-(2,3-dihydro-1H-inden-2-ylamino)propyl]-4,5-dihydro-2H-pyrazolo[3,4-f]isoquinoline-3-carboxylic acid trifluoroacetate,
2-(2-naphthyl)-5,6,8,9,10,11-hexahydro-7H-[1,4]diazepino[1′,2′:1,5]pyrazolo[3,4-f]isoquinolin-7-one,
2-{3-[(3-phenylpropyl)amino]propyl}-4,5-dihydro-2H-pyrazolo[3,4-f]isoquinoline-3-carboxylic acid trifluoroacetate,
2-(3-{[2-(4-fluorophenyl)ethyl]amino}propyl)-4,5-dihydro-2H-pyrazolo[3,4-f]isoquinoline-3-carboxylic acid trifluoroacetate,
2-{3-[(2-thien-3-ylethyl)amino]propyl}-4,5-dihydro-2H-pyrazolo[3,4-f]isoquinoline-3-carboxylic acid dihydrochloride,
5,6,9,10-tetrahydropyrazino[1′,2′:1,5]pyrazolo[3,4-f]isoquinolin-7(8H)-one,
2-[3-(glycoloylamino)propyl]-8-quinolin-3-yl-4,5-dihydro-2H-pyrazolo[3,4-f]isoquinoline-3-carboxylic acid hydrochloride,
8-quinolin-3-yl-4,5-dihydro-2H-pyrazolo[3,4-f]isoquinoline-3-carboxylic acid,
2-(3-{[2-(4-ethylphenyl)ethyl]amino}propyl)-4,5-dihydro-2H-pyrazolo[3,4-f]isoquinoline-3-carboxylic acid trifluoroacetate,
2-(3-{[2-(2-chlorophenyl)ethyl]amino}propyl)-4,5-dihydro-2H-pyrazolo[3,4-f]isoquinoline-3-carboxylic acid trifluoroacetate,
2-{3-[(2-ethylbutyl)amino]propyl}-4,5-dihydro-2H-pyrazolo[3,4-f]isoquinoline-3-carboxylic acid trifluoroacetate,
8-quinolin-3-yl-4,5-dihydro-2H-pyrazolo[3,4-f]isoquinoline-3-carboxamide,
2-{3-[(2-pyridin-4-ylethyl)amino]propyl}-4,5-dihydro-2H-pyrazolo[3,4-f]isoquinoline-3-carboxylic acid trifluoroacetate,
2-(3-{[2-(4-chlorophenyl)propyl]amino}propyl)-4,5-dihydro-2H-pyrazolo[3,4-f]isoquinoline-3-carboxylic acid trifluoroacetate,
2-(3-{[2-(3-chlorophenyl)ethyl]amino}propyl)-4,5-dihydro-2H-pyrazolo[3,4-f]isoquinoline-3-carboxylic acid trifluoroacetate,
2-[3-(glycoloylamino)propyl]-4,5-dihydro-2H-pyrazolo[3,4-f]isoquinoline-3-carboxylic acid trifluoroacetate,
2-(3-{[2-(3,4-dimethoxyphenyl)ethyl]amino}propyl)-4,5-dihydro-2H-pyrazolo[3,4-f]isoquinoline-3-carboxylic acid trifluoroacetate,
2-(1-benzofuran-2-yl)-5,6,8,9,0,11-hexahydro-7H-[1,4]diazepino[1′,2′:1,5]pyrazolo[3,4-f]isoquinolin-7-one,
2-(3-{[4-(2-aminoethyl)phenyl]amino}propyl)-4,5-dihydro-2H-pyrazolo[3,4-f]isoquinoline-3-carboxylic acid trifluoroacetate,
2-(1-naphthyl)-5,6,9,10-tetrahydropyrazino[1′,2′:1,5]pyrazolo[3,4-f]isoquinolin-7(8H)-one,
8-(3-aminopropyl)-5,6,8,9,10,11-hexahydro-7H-[1,4]diazepino[1′,2′:1,5]pyrazolo[3,4-f]isoquinolin-7-one dihydrochloride,
2-anilino-5,6,9,10-tetrahydropyrazino[1′,2′:1,5]pyrazolo[3,4-f]isoquinolin-7(8H)-one,
2-(3-aminopropyl)-8-[2-(trifluoromethyl)phenyl]-4,5-dihydro-2H-pyrazolo[3,4-f]isoquinoline-3-carboxylic acid trifluoroacetate,
9-(azidomethyl)-5,6,9,10-tetrahydropyrazino[1′,2′:1,5]pyrazolo[3,4-f]isoquinolin-7(8H)-one,
9-({[2-(4-chlorophenyl)ethyl]amino}methyl)-5,6,9,10-tetrahydropyrazino[1′,2′:1,5]pyrazolo[3,4-f]isoquinolin-7(8H)-one,
2-phenyl-5,6,8,9,10,11-hexahydro-7H-[1,4]diazepino[1′,2′:1,5]pyrazolo[3,4-f]isoquinolin-7-one,
2-[3-(pentylamino)propyl]-4,5-dihydro-2H-pyrazolo[3,4-f]isoquinoline-3-carboxylic acid trifluoroacetate,
10-(2-aminoethyl)-5,6,9,10-tetrahydropyrazino[1′,2′:1,5]pyrazolo[3,4-f]isoquinolin-7(8H)-one,
2-[3-(allylamino)propyl]-4,5-dihydro-2H-pyrazolo[3,4-f]isoquinoline-3-carboxylic acid trifluoroacetate,
2-(4-aminobutyl)-4,5-dihydro-2H-pyrazolo[3,4-f]isoquinoline-3-carboxylic acid dihydrochloride,
2-(3-{[2-(4-aminophenyl)ethyl]amino}propyl)-4,5-dihydro-2H-pyrazolo[3,4-f]isoquinoline-3-carboxylic acid trifluoroacetate,
2-[(1E)-3,3-dimethylbut-1-enyl]-5,6,9,10-tetrahydropyrazino[1′,2′:1,5]pyrazolo[3,4-f]isoquinolin-7(8H)-one trifluoroacetate,
10-(nitromethyl)-5,6,9,10-tetrahydropyrazino[1′,2′:1,5]pyrazolo[3,4-f]isoquinolin-7(8H)-one,
3-carboxy-2-[3-(methylammonio)propyl]-4,5-dihydro-2H-pyrazolo[3,4-f]isoquinolin-7-ium dichloride,
2-[3-({[(4-butoxyphenyl)amino]carbonyl}amino)propyl]-4,5-dihydro-2H-pyrazolo[3,4-f]isoquinoline-3-carboxylic acid,
2-{3-[(2-pyridin-3-ylethyl)amino]propyl}-4,5-dihydro-2H-pyrazolo[3,4-f]isoquinoline-3-carboxylic acid trifluoroacetate,
2-{3-[(2-pyridin-2-ylethyl)amino]propyl}-4,5-dihydro-2H-pyrazolo[3,4-f]isoquinoline-3-carboxylic acid trifluoroacetate,
2-{3-[(cyclopropylmethylamino]propyl}-4,5-dihydro-2H-pyrazolo[3,4-f]isoquinoline-3-carboxylic acid trifluoroacetate,
2-{3-[(2-thien-2-ylpropyl)amino]propyl}-4,5-dihydro-2H-pyrazolo[3,4-f]isoquinoline-3-carboxylic acid trifluoroacetate,
2-methoxy-5,6,9,10-tetrahydropyrazino[1′,2′:1,5]pyrazolo[3,4-f]isoquinolin-7(8H)-one,
2-[3-(dimethylamino)phenyl]-5,6,9,10-tetrahydropyrazino[1′,2′:1,5]pyrazolo[3,4-f]isoquinolin-7(8H)-one,
2-(3-{[2-(1H-pyrrol-1-yl)ethyl]amino}propyl)-4,5-dihydro-2H-pyrazolo[3,4-f]isoquinoline-3-carboxylic acid trifluoroacetate,
2-[3-(benzyloxy)propyl]-8-quinolin-3-yl-4,5-dihydro-2H-pyrazolo[3,4-f]isoquinoline-3-carboxylic acid,
2-{3-[(4-butoxybenzyl)amino]propyl}-4,5-dihydro-2H-pyrazolo[3,4-f]isoquinoline-3-carboxylic acid trifluoroacetate,
2-(1,3-benzodioxol-5-yl)-5,6,9,10-tetrahydropyrazino[1′,2′:1,5]pyrazolo[3,4-f]isoquinolin-7(8H)-one,
2-[(E)-2-(2-fluorophenyl)ethenyl]-5,6,9,10-tetrahydropyrazino[1′,2′:1,5]pyrazolo[3,4-f]isoquinolin-7(8H)-one,
2-[(1E)-hex-1-enyl]-5,6,9,10-tetrahydropyrazino[1′,2′:1,5]pyrazolo[3,4-f]isoquinolin-7(8H)-one trifluoroacetate,
2-anilino-5,6,8,9,10,11-hexahydro-7H-[1,4]diazepino[1′,2′:1,5]pyrazolo[3,4-f]isoquinolin-7-one,
5,6,8,9,10,11-hexahydro-7H-[1,4]diazepino[1′,2′:1,5]pyrazolo[3,4-f]isoquinolin-7-one,
2-chloro-5,6,9,10-tetrahydropyrazino[1′,2′:1,5]pyrazolo[3,4-f]isoquinolin-7(8H)-one,
2-[(E)-2-(4-methoxyphenyl)ethenyl]-5,6,9,10-tetrahydropyrazino[1′,2′:1,5]pyrazolo[3,4-f]isoquinolin-7(8H)-one,
2-(methylthio)-5,6,9,10-tetrahydropyrazino[1′,2′:1,5]pyrazolo[3,4-f]isoquinolin-7(8H)-one,
2-{3-[(2-furylmethyl)amino]propyl}-4,5-dihydro-2H-pyrazolo[3,4-f]isoquinoline-3-carboxylic acid trifluoroacetate,
2-azepan-1-yl-5,6,9,10-tetrahydropyrazino[1′,2′:1,5]pyrazolo[3,4-f]isoquinolin-7(8H)-one trifluoroacetate,
2-(3,6-dihydropyridin-1 (2H)-yl)-5,6,9,10-tetrahydropyrazino[1′,2′:1,5]pyrazolo[3,4-f]isoquinolin-7(8H)-one trifluoroacetate,
9-methyl-5,6,9,10-tetrahydropyrazino[1′,2′:1,5]pyrazolo[3,4-f]isoquinolin-7(8H)-one,
2-(piperidin-3-ylmethyl)-4,5-dihydro-2H-pyrazolo[3,4-f]isoquinoline-3-carboxylic acid hydrochloride,
9-(chloromethyl)-5,6,9,10-tetrahydropyrazino[1′,2′:1,5]pyrazolo[3,4-f]isoquinolin-7(8H)-one,
2-[(4-methoxybenzyl)amino]-5,6,9,10-tetrahydropyrazino[1′,2′:1,5]pyrazolo[3,4-f]isoquinolin-7(8H)-one trifluoroacetate,
2-(3-{[2-(1H-imidazol-4-yl)ethyl]amino}propyl)-4,5-dihydro-2H-pyrazolo[3,4-f]isoquinoline-3-carboxylic acid trifluoroacetate,
2-(benzylamino)-5,6,9,10-tetrahydropyrazino[1′,2′:1,5]pyrazolo[3,4-f]isoquinolin-7(8H)-one trifluoroacetate,
2-(methylthio)-5,6,8,9,10,11-hexahydro-7H-[1,4]diazepino[1′,2′:1,5]pyrazolo[3,4-f]isoquinolin-7-one,
2-{3-[(2-chlorobenzyl)amino]propyl}-4,5-dihydro-2H-pyrazolo[3,4-f]isoquinoline-3-carboxylic acid trifluoroacetate,
2-[3-(benzylamino)propyl]-4,5-dihydro-2H-pyrazolo[3,4-f]isoquinoline-3-carboxylic acid trifluoroacetate,
2-[3-({[(4-methoxyphenyl)amino]carbonyl}amino)propyl]-4,5-dihydro-2H-pyrazolo[3,4-f]isoquinoline-3-carboxylic-acid,
2-{3-[(2-phenylethyl)amino]propyl}-4,5-dihydro-2H-pyrazolo[3,4-f]isoquinoline-3-carboxylic acid trifluoroacetate,
2-{3-[(thien-2-ylmethyl)amino]propyl}-4,5-dihydro-2H-pyrazolo[3,4-f]isoquinoline-3-carboxylic acid trifluoroacetate,
2-benzyl-5,6,9,10-tetrahydropyrazino[1′,2′:1,5]pyrazolo[3,4-f]isoquinolin-7(8H)-one,
5,6,8,9,10,11-hexahydro-7H-[1,4]diazepino[1′,2′:1,5]pyrazolo[3,4-f]isoquinolin-7-one,
2-{3-[(4-chlorobenzyl)amino]propyl}-4,5-dihydro-2H-pyrazolo[3,4-f]isoquinoline-3-carboxylic acid trifluoroacetate,
2-{3-[(2-phenylpropyl)amino]propyl}-4,5-dihydro-2H-pyrazolo[3,4-f]isoquinoline-3-carboxylic acid trifluoroacetate,
7-oxo-5,6,7,8,9,10-hexahydropyrazino[1′,2′:1,5]pyrazolo[3,4-f]isoquinoline-9-carboxamide trifluoroacetate,
2-(3-hydroxypropyl)-4,5-dihydro-2H-pyrazolo[3,4-f]isoquinoline-3-carboxylic acid,
2-(1,3-dihydro-2H-isoindol-2-yl)-5,6,9,10-tetrahydropyrazino[1′,2′:1,5]pyrazolo[3,4-f]isoquinolin-7(8H)-one trifluoroacetate,
2-{3-[(4-aminophenyl)amino]propyl}-4,5-dihydro-2H-pyrazolo[3,4-f]isoquinoline-3-carboxylic acid trifluoroacetate,
2-(4-hydroxypiperidin-1-yl)-5,6,9,10-tetrahydropyrazino[1′,2′:1,5]pyrazolo[3,4-f]isoquinolin-7(8H)-one trifluoroacetate,
2-{3-[(1H-imidazol-5-ylmethyl)amino]propyl}-4,5-dihydro-2H-pyrazolo[3,4-f]isoquinoline-3-carboxylic acid trifluoroacetate,
2-{3-[(3-chlorobenzyl)amino]propyl}-4,5-dihydro-2H-pyrazolo[3,4-f]isoquinoline-3-carboxylic acid trifluoroacetate,
2-{3-[(pyridin-3-ylcarbonyl)amino]propyl}-4,5-dihydro-2H-pyrazolo[3,4-f]isoquinoline-3-carboxylic acid trifluoroacetate,
5-dihydro-2H-pyrazolo[3,4-f]isoquinoline-3-carboxamide,
2-[3-(cyclopentylamino)propyl]-4,5-dihydro-2H-pyrazolo[3,4-f]isoquinoline-3-carboxylic acid dihydrochloride,
2-(3-{[2-(1H-indol-3-yl)ethyl]amino}propyl)-4,5-dihydro-2H-pyrazolo[3,4-f]isoquinoline-3-carboxylic acid,
10-(3-aminopropyl)-5,6,9,10-tetrahydropyrazino[1′,2′:1,5]pyrazolo[3,4-f]isoquinolin-7(8H)-one,
9-(1,2-dihydroxyethyl)-5,6,9,10-tetrahydropyrazino[1′,2′:1,5]pyrazolo[3,4-f]isoquinolin-7(8H)-one,
2-morpholin-4-yl-5,6,9,10-tetrahydropyrazino[1′,2′:1,5]pyrazolo[3,4-f]isoquinolin-7(8H)-one trifluoroacetate,
ethyl 2-(2-aminoethyl)-4,5-dihydro-2H-pyrazolo[3,4-f]isoquinoline-3-carboxylate dihydrochloride,
2-allyl-4,5-dihydro-2H-pyrazolo[3,4-f]isoquinoline-3-carboxylic acid,
2-quinolin-8-yl-5,6,9,10-tetrahydropyrazino[1′,2′:1,5]pyrazolo[3,4-f]isoquinolin-7(8H)-one,
lithium 2-[3-(2,3-dihydro-1H-inden-2-ylamino)-2-hydroxypropyl]-4,5-dihydro-2H-pyrazolo[3,4-f]isoquinoline-3-carboxylate,
2-(2-hydroxyethyl)-4,5-dihydro-2H-pyrazolo[3,4-f]isoquinoline-3-carboxylic acid,
2-{3-[(2-pyrrolidin-1-ylethyl)amino]propyl}-4,5-dihydro-2H-pyrazolo[3,4-f]isoquinoline-3-carboxylic acid trifluoroacetate,
2-(3,4-dihydroisoquinolin-2(1H)-yl)-5,6,9,10-tetrahydropyrazino[1′,2′:1,5]pyrazolo[3,4-f]isoquinolin-7(8H)-one trifluoroacetate,
2-(3-{[(benzylamino)carbonyl]amino}propyl)-4,5-dihydro-2H-pyrazolo[3,4-f]isoquinoline-3-carboxylic acid,
2-[3-(dimethylamino)propyl]-4,5-dihydro-2H-pyrazolo[3,4-f]isoquinoline-3-carboxylic acid trifluoroacetate,
10-(3-{[2-(4-chlorophenyl)ethyl]amino}propyl)-5,6,9,10-tetrahydropyrazino[1′,2′:1,5]pyrazolo[3,4-f]isoquinolin-7(8H)-one,
2-(3-aminopropyl)-6-chloro-4,5-dihydro-2H-pyrazolo[3,4-f]isoquinoline-3-carboxylic acid trifluoroacetate,
methyl 7-oxo-5,6,7,8,9,10-hexahydropyrazino[1′,2′:1,5]pyrazolo[3,4-f]isoquinoline-9-carboxylate,
2-[2-(glycoloylamino)ethyl]-4,5-dihydro-2H-pyrazolo[3,4-f]isoquinoline-3-carboxylic acid hydrochloride,
2-[2-(isopropylamino)ethyl]-4,5-dihydro-2H-pyrazolo[3,4-f]isoquinoline-3-carboxylic acid hydrochloride,
2-(3-cyanopropyl)-4,5-dihydro-2H-pyrazolo[3,4-f]isoquinoline-3-carboxylic acid,
2-[3-({[(3-cyanophenyl)amino]carbonyl}amino)propyl]-4,5-dihydro-2H-pyrazolo[3,4-f]isoquinoline-3-carboxylic acid,
9-(aminomethyl)-5,6,9,10-tetrahydro-7H-[1,4]oxazino[4′,3′:1,5]pyrazolo[3,4-f]isoquinolin-7-one trifluoroacetate,
4-methyl-5,6,8,9,10,11-hexahydro-7H-[1,4]diazepino[1′,2′:1,5]pyrazolo[3,4-f]isoquinolin-7-one trifluoroacetate,
2-propyl-4,5-dihydro-2H-pyrazolo[3,4-f]isoquinoline-3-carboxylic acid,
2-(3-bromopropyl)-4,5-dihydro-2H-pyrazolo[3,4-f]isoquinoline-3-carboxylic acid trifluoroacetate,
2-pyrrolidin-1-yl-5,6,9,10-tetrahydropyrazino[1′,2′:1,5]pyrazolo[3,4-f]isoquinolin-7(8H)-one,
2-[3-(isopropylamino)propyl]-4,5-dihydro-2H-pyrazolo[3,4-f]isoquinoline-3-carboxylic acid hydrochloride,
2-(3-amino-3-carboxypropyl)-4,5-dihydro-2H-pyrazolo[3,4-f]isoquinoline-3-carboxylic acid trifluoroacetate,
2-{3-[(aminocarbonyl)amino]propyl}-4,5-dihydro-2H-pyrazolo[3,4-f]isoquinoline-3-carboxylic acid,
9-{[(4-chlorobenzyl)amino]methyl}-5,6,9,10-tetrahydropyrazino[1′,2′:1,5]pyrazolo[3,4-f]isoquinolin-7(8H)-one,
4-chloro-5,6,9,10-tetrahydropyrazino[1′,2′:1,5]pyrazolo[3,4-f]isoquinolin-7(8H)-one,
7-oxo-2-quinolin-3-yl-5,6,7,8,9,10-hexahydropyrazino[1′,2′:1,5]pyrazolo[3,4-f]isoquinoline-9-carboxamide,
2-{3-[(methylsulfonyl)amino]propyl}-4,5-dihydro-2H-pyrazolo[3,4-f]isoquinoline-3-carboxylic acid trifluoroacetate,
lithium 2-{4-[(2-ethylbutyl)amino]butyl}-4,5-dihydro-2H-pyrazolo[3,4-f]isoquinoline-3-carboxylate,
2-(3-{[(4-methoxyphenyl)acetyl]amino}propyl)-4,5-dihydro-2H-pyrazolo[3,4-f]isoquinoline-3-carboxylic acid trifluoroacetate,
7-oxo-6,7,8,9,10,11-hexahydro-5H-[1,4]diazepino[1′,2′:1,5]pyrazolo[3,4-f]isoquinoline-9-carbonitrile,
2-{3-[(isopropylsulfonyl)amino]propyl}-4,5-dihydro-2H-pyrazolo[3,4-f]isoquinoline-3-carboxylic acid trifluoroacetate,
3-[3-(chloromethyl)-4,5-dihydro-2H-pyrazolo[3,4-f]isoquinolin-2-yl]propan-1-amine hydrochloride,
2-(3,3-dimethylbutyl)-5,6,9,10-tetrahydropyrazino[1′,2′:1,5]pyrazolo[3,4-f]isoquinolin-7(8H)-one trifluoroacetate,
2-thiomorpholin-4-yl-5,6,9,10-tetrahydropyrazino[1′,2′:1,5]pyrazolo[3,4-f]isoquinolin-7(8H)-one trifluoroacetate,
ethyl 2-(2-aminoethyl)-8-chloro-4,5-dihydro-2H-pyrazolo[3,4-f]isoquinoline-3-carboxylate,
2-[4-(benzyloxy)phenyl]-5,6,9,10-tetrahydropyrazino[1′,2′:1,5]pyrazolo[3,4-f]isoquinolin-7(8H)-one-lithium 2-{2-hydroxy-3-[(thien-2-ylmethyl)amino]propyl}-4,5-dihydro-2H-pyrazolo[3,4-f]isoquinoline-3-carboxylate,
2-(3-amino-3-cyanopropyl)-4,5-dihydro-2H-pyrazolo[3,4-f]isoquinoline-3-carboxylic acid trifluoroacetate,
2-[3-(pyridin-3-ylamino)propyl]-4,5-dihydro-2H-pyrazolo[3,4-f]isoquinoline-3-carboxylic acid trifluoroacetate,
2-[3-({[(ethoxycarbonyl)amino]carbonyl}amino)propyl]-4,5-dihydro-2H-pyrazolo[3,4-f]isoquinoline-3-carboxylic acid trifluoroacetate,
2-{3-[4-(aminocarbonyl)piperidin-1-yl]propyl}-4,5-dihydro-2H-pyrazolo[3,4-f]isoquinoline-3-carboxylic acid trifluoroacetate,
2-(4-amino-4-oxobutyl)-4,5-dihydro-2H-pyrazolo[3,4-f]isoquinoline-3-carboxylic acid trifluoroacetate,
8-quinolin-3-yl-4,5-dihydro-2H-pyrazolo[3,4-f]isoquinoline,
2-(3-{[(butylamino)carbonyl]amino}propyl)-4,5-dihydro-2H-pyrazolo[3,4-f]isoquinoline-3-carboxylic acid trifluoroacetate,
3-(2-furyl)-4,5-dihydro-2H-pyrazolo[3,4-f]isoquinoline,
4-anilino-5,6,9,10-tetrahydropyrazino[1′,2′:1,5]pyrazolo[3,4-f]isoquinolin-7(8H)-one,
8-(3-aminopropyl)-5,6,9,10-tetrahydropyrazino[1′,2′:1,5]pyrazolo[3,4-f]isoquinolin-7(8H)-one dihydrochloride,
2-(3-{[(allylamino)carbonyl]amino}propyl)-4,5-dihydro-2H-pyrazolo[3,4-f]isoquinoline-3-carboxylic acid trifluoroacetate,
2-{3-[(2,2,2-trifluoroethyl)amino]propyl}-4,5-dihydro-2H-pyrazolo[3,4-f]isoquinoline-3-carboxylic acid trifluoroacetate,
5,6,9,10-tetrahydropyrido[1′,2′:1,5]pyrazolo[3,4-f]isoquinolin-7(8H)-one,
2-{[2-(dimethylamino)ethyl]amino}-5,6,9,10-tetrahydropyrazino[1′,2′:1,5]pyrazolo[3,4-f]isoquinolin-7(8H)-one trifluoroacetate,
2-{2-[(2-ethylbutyl)amino]ethyl}-4,5-dihydro-2H-pyrazolo[3,4-f]isoquinoline-3-carboxylic acid,
ethyl 2-(2-aminoethyl)-8-ethynyl-4,5-dihydro-2H-pyrazolo[3,4-f]isoquinoline-3-carboxylate hydrochloride,
2-[2-(4-chlorophenyl)ethyl]-5,6,9,10-tetrahydropyrazino[1′,2′:1,5]pyrazolo[3,4-f]isoquinolin-7(8H)-one trifluoroacetate,
4,5-dihydro-2H-pyrazolo[3,4-f]isoquinoline-3-carboxylic acid,
2-piperazin-1-yl-5,6,9,10-tetrahydropyrazino[1′,2′:1,5]pyrazolo[3,4-f]isoquinolin-7(8H)-one trifluoroacetate,
2-{3-[(thien-2-ylacetyl)amino]propyl}-4,5-dihydro-2H-pyrazolo[3,4-f]isoquinoline-3-carboxylic acid trifluoroacetate hydrochloride,
2-[3-(benzoylamino)propyl]-4,5-dihydro-2H-pyrazolo[3,4-f]isoquinoline-3-carboxylic acid trifluoroacetate,
2-[3-(dimethylamino)-2-hydroxypropyl]-N,N-dimethyl-4,5-dihydro-2H-pyrazolo[3,4-f]isoquinoline-3-carboxamide trifluoroacetate,
2-[(2E)-2-(hydroxyimino)ethyl]-4,5-dihydro-2H-pyrazolo[3,4-f]isoquinoline-3-carboxylic acid,
2-allyl-4,5-dihydro-2H-pyrazolo[3,4-f]isoquinoline-3-carboxylic acid 7-oxide,
2-{3-[({[(4-methylphenyl)sulfonyl]amino}carbonyl)amino]propyl}-4,5-dihydro-2H-pyrazolo[3,4-f]isoquinoline-3-carboxylic acid trifluoroacetate,
2-(3-aminopropyl)-6-(methylthio)-4,5-dihydro-2H-pyrazolo[3,4-f]isoquinoline-3-carboxylic acid trifluoroacetate,
2-(3-methylpiperidin-1-yl)-5,6,9,10-tetrahydropyrazino[1′,2′-: 1,5]pyrazolo[3,4-f]isoquinolin-7(8H)-one trifluoroacetate,
3-(methylthio)-4,5-dihydro-2H-pyrazolo[3,4-f]isoquinoline,
2-(3-aminopropyl)-6-phenyl-4,5-dihydro-2H-pyrazolo[3,4-q]isoquinoline-3-carboxylic acid trifluoroacetate,
lithium 2-{3-[4-(aminocarbonyl)piperidin-1-yl]-2-hydroxypropyl}-4,5-dihydro-2H-pyrazolo[3,4-f]isoquinoline-3-carboxylate,
2-(3-{bis[3-(5-methyl-2-furyl)butyl]amino}propyl)-4,5-dihydro-2H-pyrazolo[3,4-f]isoquinoline-3-carboxylic acid dihydrochloride,
2-[4-(benzyloxy)phenyl]-5,6,8,9,10,11-hexahydro-7H-[1,4]diazepino[1′,2′:1,5]pyrazolo[3,4-f]isoquinolin-7-one,
2-(4-chlorobenzyl)-5,6,9,10-tetrahydropyrazino[1′,2′:1,5]pyrazolo[3,4-f]isoquinolin-7(8H)-one trifluoroacetate,
3-(difluoromethyl)-4,5-dihydro-2H-pyrazolo[3,4-f]isoquinoline,
2-(4-methylpiperazin-1-yl)-5,6,9,10-tetrahydropyrazino[1′,2′:1,5]pyrazolo[3,4-f]isoquinolin-7(8H)-one trifluoroacetate,
2-quinolin-5-yl-5,6,9,10-tetrahydropyrazino[1′,2′:1,5]pyrazolo[3,4-f]isoquinolin-7(8H)-one,
2-(3-aminopropyl)-7-hydroxy-8-(3-nitrophenyl)-4,5-dihydro-2H-benzo[g]indazole-3-carboxylic acid trifluoroacetate,
2-(2-aminoethyl)-7-hydroxy-8-(3-nitrophenyl)-4,5-dihydro-2H-benzo[g]indazole-3-carboxylic acid trifluoroacetate,
3-hydroxy-2-(3-nitrophenyl)-5,6,8,9,10,11-hexahydro-7H-benzo[g][1,4]diazepino[1,2-b]indazol-7-one trifluoroacetate,
2-(3-aminopropyl)-7-hydroxy-4,5-dihydro-2H-benzo[g]indazole-3-carboxylic acid dihydrochloride,
2-(2-aminoethyl)-8-bromo-7-hydroxy-4,5-dihydro-2H-benzo[g]indazole-3-carboxylic acid trifluoroacetate,
2-(3-aminopropyl)-8-[2-(4-chlorophenyl)ethyl]-7-hydroxy-4,5-dihydro-2H-benzo[g]indazole-3-carboxylic acid trifluoroacetate,
3-hydroxy-2-(3-nitrophenyl)-5,6,9,10-tetrahydrobenzo[g]pyrazino[1,2-b]indazol-7(8H)-one hydrobromide,
2-(3-aminopropyl)-8-bromo-7-hydroxy-4,5-dihydro-2H-benzo[g]indazole-3-carboxylic acid hydrobromide,
2-(3-aminopropyl)-7-hydroxy-8-(4-nitrophenyl)-4,5-dihydro-2H-benzo[g]indazole-3-carboxylic acid trifluoroacetate,
2-(3-aminopropyl)-8-(3-cyanophenyl)-7-hydroxy-4,5-dihydro-2H-benzo[g]indazole-3-carboxylic acid trifluoroacetate,
2-(3-aminopropyl)-7-hydroxy-8-[3-(trifluoromethyl)phenyl]-4,5-dihydro-2H-benzo[g]indazole-3-carboxylic acid trifluoroacetate,
2-(3-aminopropyl)-7-hydroxy-8-(3,3,3-trifluoropropyl)-4,5-dihydro-2H-benzo[g]indazole-3-carboxylic acid trifluoroacetate,
2-(3-aminopropyl)-8-(3-chlorophenyl)-7-hydroxy-4,5-dihydro-2H-benzo[g]indazole-3-carboxylic acid trifluoroacetate,
2-(3-aminopropyl)-7-hydroxy-5-methyl-4,5-dihydro-2H-benzo[g]indazole-3-carboxylic acid trifluoroacetate,
2-(3-aminopropyl)-8-(4-chlorophenyl)-7-hydroxy-4,5-dihydro-2H-benzo[g]indazole-3-carboxylic acid trifluoroacetate,
2-(3-aminopropyl)-7-hydroxy-8-[3-(methylsulfonyl)phenyl]-4,5-dihydro-2H-benzo[g]indazole-3-carboxylic acid trifluoroacetate,
2-(3-aminopropyl)-8-(3,4-difluorophenyl)-7-hydroxy-4,5-dihydro-2H-benzo[g]indazole-3-carboxylic acid trifluoroacetate,
3-hydroxy-5,6,8,9,10,11-hexahydro-7H-benzo[g][1,4]diazepino[1,2-b]indazol-7-one,
8-(3-aminophenyl)-2-(3-aminopropyl)-7-hydroxy-4,5-dihydro-2H-benzo[g]indazole-3-carboxylic acid trifluoroacetate,
6-bromo-7-hydroxy-4,5-dihydro-2H-benzo[g]indazole-3-carboxamide,
2-(3-aminopropyl)-7-hydroxy-8-[4-(methylamino)phenyl]-4,5-dihydro-2H-benzo[g]indazole-3-carboxylic acid trifluoroacetate,
2-(3-aminopropyl)-8-(4-tert-butylphenyl)-7-hydroxy-4,5-dihydro-2H-benzo[g]indazole-3-carboxylic acid trifluoroacetate,
2-(3-aminopropyl)-7-hydroxy-8-pyridin-4-yl-4,5-dihydro-2H-benzo[g]indazole-3-carboxylic acid trifluoroacetate,
7-hydroxy-4,5-dihydro-2H-benzo[g]indazole-3-carboxylic acid,
2-(3-aminopropyl)-7-hydroxy-8-isobutyl-4,5-dihydro-2H-benzo[g]indazole-3-carboxylic acid trifluoroacetate,
2-(3-aminopropyl)-8-(4-carboxy-3,3-dimethylbutyl)-7-hydroxy-4,5-dihydro-2H-benzo[g]indazole-3-carboxylic acid trifluoroacetate,
8-amino-2-(3-aminopropyl)-7-hydroxy-4,5-dihydro-2H-benzo[g]indazole-3-carboxylic acid,
7,8-dihydroxy-4,5-dihydro-2H-benzo[g]indazole-3-carboxylic acid,
2-(3-aminopropyl)-8-benzyl-7-hydroxy-4,5-dihydro-2H-benzo[g]indazole-3-carboxylic acid trifluoroacetate,
2-(3-aminopropyl)-8-(2-chlorophenyl)-7-hydroxy-4,5-dihydro-2H-benzo[g]indazole-3-carboxylic acid trifluoroacetate,
2-allyl-7-hydroxy-4,5-dihydro-2H-benzo[g]indazole-3-carboxylic acid,
2-{3-[(tert-butoxycarbonyl)amino]propyl}-7-methoxy-8-(4-nitrophenyl)-4,5-dihydro-2H-benzo[g]indazole-3-carboxylic acid,
7-hydroxy-2-methyl-4,5-dihydro-2H-benzo[g]indazole-3-carboxylic acid,
2-(3-aminopropyl)-8-(sec-butylamino)-7-hydroxy-4,5-dihydro-2H-benzo[g]indazole-3-carboxylic acid,
2-(3-aminopropyl)-8-(3-carboxyphenyl)-7-hydroxy-4,5-dihydro-2H-benzo[g]indazole-3-carboxylic acid trifluoroacetate,
2-{3-[(tert-butoxycarbonyl)amino]propyl}-7-methoxy-8-(3-nitrophenyl)-4,5-dihydro-2H-benzo[g]indazole-3-carboxylic acid,
2-{3-[(tert-butoxycarbonyl)amino]propyl}-8-(3-cyanophenyl)-7-methoxy-4,5-dihydro-2H-benzo[g]indazole-3-carboxylic acid,
7-hydroxy-1-methyl-4,5-dihydro-1H-benzo[g]indazole-3-carboxylic acid,
2-{3-[(tert-butoxycarbonyl)amino]propyl}-8-[2-(4-chlorophenyl)ethyl]-7-methoxy-4,5-dihydro-2H-benzo[g]indazole-3-carboxylic acid,
2-{3-[(tert-butoxycarbonyl)amino]propyl}-7-methoxy-8-[3-(trifluoromethyl)phenyl]-4,5-dihydro-2H-benzo[g]indazole-3-carboxylic acid,
2-{3-[(tert-butoxycarbonyl)amino]propyl}-8-(4-tert-butylphenyl)-7-methoxy-4,5-dihydro-2H-benzo[g]indazole-3-carboxylic acid,
2-(3-aminopropyl)-6-chloro-7-hydroxy-8-(3-nitrophenyl)-4,5-dihydro-2H-benzo[g]indazole-3-carboxylic acid,
2-{2-[(tert-butoxycarbonyl)amino]ethyl}-7-methoxy-8-(3-nitrophenyl)-4,5-dihydro-2H-benzo[g]indazole-3-carboxylic acid,
2-amino-3-hydroxy-5,6,8,9,10,11-hexahydro-7H-benzo[g][1,4]diazepino[1,2-b]indazol-7-one trifluoroacetate,
2-{3-[(tert-butoxycarbonyl)amino]propyl}-8-(3,4-difluorophenyl)-7-methoxy-4,5-dihydro-2H-benzo[g]indazole-3-carboxylic acid,
methyl 8-[(dimethylamino)sulfonyl]-7-hydroxy-4,5-dihydro-2H-benzo[g]indazole-3-carboxylate,
7-hydroxy-5-methyl-4,5-dihydro-2H-benzo[g]indazole-3-carboxylic acid, and
2-{3-[(tert-butoxycarbonyl)amino]propyl}-8-(4-chlorophenyl)-7-methoxy-4,5-dihydro-2H-benzo[g]indazole-3-carboxylic acid.

18. The cyclic pyrazole MK-2 inhibitor according to claim 1, wherein the MK-2 inhibitor has an MK-2 IC₅₀ of less than 50 and is selected from the group consisting of:
2-(3-aminopropyl)-8-(methylthio)-2,4,5,6-tetrahydropyrazolo[3,4-e]indazole-3-carboxylic acid dihydrochloride,
2-(3-aminopropyl)-2,4,5,6-tetrahydropyrazolo[3,4-e]indazole-3-carboxylic acid hydrochloride,
2-(3-{[2-(4-bromophenyl)ethyl]amino}propyl)-2,4,5,6-tetrahydropyrazolo[3,4-e]indazole-3-carboxylic acid hydrochloride,
2-(2-aminoethyl)-2,4,5,6-tetrahydropyrazolo[3,4-e]indazole-3-carboxylic acid hydrochloride,
8-(allylthio)-2-(3-aminopropyl)-2,4,5,6-tetrahydropyrazolo[3,4-e]indazole-3-carboxylic acid dihydrochloride,
2-(3-aminopropyl)-8-(benzylthio)-2,4,5,6-tetrahydropyrazolo[3,4-e]indazole-3-carboxylic acid dihydrochloride,
2-{3-[(2-thien-2-ylethyl)amino]propyl}-2,4,5,6-tetrahydropyrazolo[3,4-e]indazole-3-carboxylic acid,
2-{3-[(2-thien-3-ylethyl)amino]propyl}-2,4,5,6-tetrahydropyrazolo[3,4-e]indazole-3-carboxylic acid hydrochloride,
ethyl 2-(3-{[2-(4-bromophenyl)ethyl]amino}propyl)-2,4,5,6-tetrahydropyrazolo[3,4-e]indazole-3-carboxylate,
2-(4-aminobutyl)-2,4,5,6-tetrahydropyrazolo[3,4-e]indazole-3-carboxylic acid dihydrochloride,
1-(methylthio)-4,5,7,8,9,10-hexahydro[1,4]diazepino[1,2-b]pyrazolo[3,4-g]indazol-6(3H)-one,
1-(allylthio)-4,5,7,8,9,10-hexahydro[1,4]diazepino[1,2-b]pyrazolo[3,4-g]indazol-6(3H)-one,
4,5,7,8,9,10-hexahydro[1,4]diazepino[1,2-b]pyrazolo[3,4-g]indazol-6(3H)-one,
4,5,8,9-tetrahydro-3H-pyrazino[1,2-b]pyrazolo[3,4-g]indazol-6(7H)-one,
2-allyl-2,4,5,6-tetrahydropyrazolo[3,4-e]indazole-3-carboxylic acid hydrochloride,
1-(benzylthio)-4,5,7,8,9,10-hexahydro[1,4]diazepino[1,2-b]pyrazolo[3,4-g]indazol-6(3H)-one,
2,4,5,6-tetrahydropyrazolo[3,4-e]indazole-3-carboxamide,
8-(methylthio)-2,4,5,6-tetrahydropyrazolo[3,4-e]indazole-3-carboxamide,
1-{1-[(benzyloxy)carbonyl]piperidin-4-yl}-6-trityl-1,4,5,6-tetrahydropyrazolo[3,4-e]indazole-3-carboxylic acid,
2-(3-aminopropyl)-8-quinolin-3-yl-4,5-dihydro-2H-pyrazolo[4,3-h]quinazoline-3-carboxylic acid dihydrochloride,
2-(3-aminopropyl)-8-(1,3-benzodioxol-5-yl)-4,5-dihydro-2H-pyrazolo[4,3-h]quinazoline-3-carboxylic acid hydrochloride,
2-(3-aminopropyl)-8-phenyl-4,5-dihydro-2H-pyrazolo[4,3-h]quinazoline-3-carboxylic acid hydrochloride,
2-quinolin-3-yl-5,6,8,9,10,1-hexahydro-7H-[1,4]diazepino[1′,2′:1,5]pyrazolo[4,3-h]quinazolin-7-one,
2-pyridin-3-yl-5,6,8,9,10,11-hexahydro-7H-[1,4]diazepino[1′,2′:1,5]pyrazolo[4,3-h]quinazolin-7-one,
8-quinolin-3-yl-2-[3-(tritylamino)propyl]-4,5-dihydro-2H-pyrazolo[4,3-h]quinazoline-3-carboxylic acid hydrochloride,
2-(1,3-benzodioxol-5-yl)-5,6,8,9,10,11-hexahydro-7H-[1,4]diazepino[1′,2′:1,5]pyrazolo[4,3-h]quinazolin-7-one,
2-(4-methoxyphenyl)-5,6,8,9,10,11-hexahydro-7H-[1,4]diazepino[1′,2′:1,5]pyrazolo[4,3-h]quinazolin-7-one,
2-pyridin-4-yl-5,6,8,9,10,11-hexahydro-7H-[1,4]diazepino[1′,2′:1,5]pyrazolo[4,3-h]quinazolin-7-one hydrochloride,
2-(3-aminopropyl)-8-quinolin-3-yl-4,5-dihydro-2H-pyrazolo[3,4-f]isoquinoline-3-carboxylic acid trifluoroacetate,
2-(3-aminopropyl)-8-(3-nitrophenyl)-4,5-dihydro-2H-pyrazolo[3,4-f]isoquinoline-3-carboxylic acid trifluoroacetate,
2-(3-aminopropyl)-8-(4-hydroxyphenyl)-4,5-dihydro-2H-pyrazolo[3,4-f]isoquinoline-3-carboxylic acid hydrobromide,
2-(3-aminopropyl)-8-(3-hydroxyphenyl)-4,5-dihydro-2H-pyrazolo[3,4-q]isoquinoline-3-carboxylic acid hydrobromide,
2-(3-aminopropyl)-8-(2-naphthyl)-4,5-dihydro-2H-pyrazolo[3,4-f]isoquinoline-3-carboxylic acid trifluoroacetate,
2-(3-aminopropyl)-8-(3,5-difluorophenyl)-4,5-dihydro-2H-pyrazolo[3,4-f]isoquinoline-3-carboxylic acid trifluoroacetate,
2-(3-aminopropyl)-8-(1,3-benzodioxol-5-yl)-4,5-dihydro-2H-pyrazolo[3,4-f]isoquinoline-3-carboxylic acid trifluoroacetate,
2-(3-aminopropyl)-8-(3-cyanophenyl)-4,5-dihydro-2H-pyrazolo[3,4-f]isoquinoline-3-carboxylic acid trifluoroacetate,
9-(hydroxymethyl)-2-quinolin-3-yl-5,6,9,10-tetrahydropyrazino[1′,2′:1,5]pyrazolo[3,4-f]isoquinolin-7(8H)-one trifluoroacetate,
2-(3-aminopropyl)-8-(4-methoxyphenyl)-4,5-dihydro-2H-pyrazolo[3,4-f]isoquinoline-3-carboxylic acid trifluoroacetate,
2-(3-aminopropyl)-8-[3-(methylsulfonyl)phenyl]-4,5-dihydro-2H-pyrazolo[3,4-f]isoquinoline-3-carboxylic acid trifluoroacetate,
2-(3-aminopropyl)-8-[3-(trifluoromethyl)phenyl]-4,5-dihydro-2H-pyrazolo[3,4-f]isoquinoline-3-carboxylic acid trifluoroacetate,
2-(1H-imidazol-1-yl)-5,6,9,10-tetrahydropyrazino[1′,2′:1,5]pyrazolo[3,4-f]isoquinolin-7(8H)-one trifluoroacetate,
2-(3-aminopropyl)-8-(3-methoxyphenyl)-4,5-dihydro-2H-pyrazolo[3,4-f]isoquinoline-3-carboxylic acid trifluoroacetate,
2-(3-aminopropyl)-8-[4-(trifluoromethyl)phenyl]-4,5-dihydro-2H-pyrazolo[3,4-f]isoquinoline-3-carboxylic acid trifluoroacetate,
2-(3-aminopropyl)-8-anilino-4,5-dihydro-2H-pyrazolo[3,4-f]isoquinoline-3-carboxylic acid,
2-(3-aminopropyl)-8-(3,4-difluorophenyl)-4,5-dihydro-2H-pyrazolo[3,4-f]isoquinoline-3-carboxylic acid trifluoroacetate,
2-(3-{[2-(4′-carboxy-1,1′-biphenyl-4-yl)ethyl]amino}propyl)-4,5-dihydro-2H-pyrazolo[3,4-f]isoquinoline-3-carboxylic acid trifluoroacetate,
2-(4-hydroxyphenyl)-5,6,9,10-tetrahydropyrazino[1′,2′:1,5]pyrazolo[3,4-f]isoquinolin-7(8H)-one trifluoroacetate,
2-propyl-8-quinolin-3-yl-4,5-dihydro-2H-pyrazolo[3,4-f]isoquinoline-3-carboxylic acid,
2-[3-({2-[3′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
2-(3-aminopropyl)-8-(4-tert-butylphenyl)-4,5-dihydro-2H-pyrazolo[3,4-f]isoquinoline-3-carboxylic acid trifluoroacetate,
2-[3-({2-[4-(3-furyl)phenyl]ethyl}amino)propyl]-4,5-dihydro-2H-pyrazolo[3,4-f]isoquinoline-3-carboxylic acid trifluoroacetate,
2-(3-{[2-(3′-chloro-1,1′-biphenyl-4-yl)ethyl]amino}propyl)-4,5-dihydro-2H-pyrazolo[3,4-f]isoquinoline-3-carboxylic acid trifluoroacetate,
2-(4-methoxyphenyl)-5,6,8,9,10,11-hexahydro-7H-[1,4]diazepino[1′,2′:1,5]pyrazolo[3,4-f]isoquinolin-7-one,
2-[3-({2-[4′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
2-(3-hydroxyphenyl)-5,6,9,10-tetrahydropyrazino[1′,2′:1,5]pyrazolo[3,4-f]isoquinolin-7(8H)-one,
2-(3-aminopropyl)-N-hydroxy-8-quinolin-3-yl-4,5-dihydro-2H-pyrazolo[3,4-f]isoquinoline-3-carboxamide hydrochloride,
2-[(E)-2-(4-hydroxyphenyl)ethenyl]-5,6,9,10-tetrahydropyrazino[1′,2′:1,5]pyrazolo[3,4-f]isoquinolin-7(8H)-one trifluoroacetate,
2-quinolin-3-yl-5,6,8,9,10,11-hexahydro-7H-[1,4]diazepino[1′,2′-: 1,5]pyrazolo[3,4-f]isoquinolin-7-one,
2-(4-hydroxyphenyl)-5,6,8,9,10,11-hexahydro-7H-[1,4]diazepino[1′,2′:1,5]pyrazolo[3,4-f]isoquinolin-7-one,
2-(3-{[2-(2′-chloro-1,1′-biphenyl-4-yl)ethyl]amino}propyl)-4,5-dihydro-2H-pyrazolo[3,4-f]isoquinoline-3-carboxylic acid trifluoroacetate,
2-(3-{[2-(4′-tert-butyl-1,1′-biphenyl-4-yl)ethyl]amino}propyl)-4,5-dihydro-2H-pyrazolo[3,4-f]isoquinoline-3-carboxylic acid trifluoroacetate,
2-(3-{[2-(3,4-dichlorophenyl)ethyl]amino}propyl)-4,5-dihydro-2H-pyrazolo[3,4-25 f]isoquinoline-3-carboxylic acid trifluoroacetate,
2-(3-{[3-(3-chlorophenyl)propyl]amino}propyl)-4,5-dihydro-2H-pyrazolo[3,4-f]isoquinoline-3-carboxylic acid trifluoroacetate,
2-[(E)-2-phenylethenyl]-5,6,9,10-tetrahydropyrazino[1′,2′:1,5]pyrazolo[3,4-f]isoquinolin-7(8H)-one trifluoroacetate,
2-(3-{[2-(4-pyridin-4-ylphenyl)ethyl]amino}propyl)-4,5-dihydro-2H-pyrazolo[3,4-f]isoquinoline-3-carboxylic acid trifluoroacetate,
2-(3-{[3-(4-bromophenyl)propyl]amino}propyl)-4,5-dihydro-2H-pyrazolo[3,4-f]isoquinoline-3-carboxylic acid trifluoroacetate,
2-(3-{[3-(4-tert-butylphenyl)propyl]amino}propyl)-4,5-dihydro-2H-pyrazolo[3,4-f]isoquinoline-3-carboxylic acid trifluoroacetate,
2-(3-{[2-(3′-isopropyl-1,1′-biphenyl-4-yl)ethyl]amino}propyl)-4,5-dihydro-2H-pyrazolo[3,4-f]isoquinoline-3-carboxylic acid trifluoroacetate,
2-{3-[(2-thien-2-ylethyl)amino]propyl}-4,5-dihydro-2H-pyrazolo[3,4-f]isoquinoline-3-carboxylic acid trifluoroacetate,
2-[4-(dimethylamino)phenyl]-5,6,9,10-tetrahydropyrazino[1′,2′:1,5]pyrazolo[3,4-f]isoquinolin-7(8H)-one,
2-(3-{[2-(1,1′-biphenyl-4-yl)ethyl]amino}propyl)-4,5-dihydro-2H-pyrazolo[3,4-f]isoquinoline-3-carboxylic acid trifluoroacetate,
2-(3-methoxyphenyl)-5,6,9,10-tetrahydropyrazino[1′,2′:1,5]pyrazolo[3,4-f]isoquinolin-7(8H)-one,
2-(3-{[2-(4-bromophenyl)ethyl]amino}propyl)-4,5-dihydro-2H-pyrazolo[3,4-f]isoquinoline-3-carboxylic acid trifluoroacetate,
2-(3-{[2-(2,4-dichlorophenyl)ethyl]amino}propyl)-4,5-dihydro-2H-pyrazolo[3,4-f]isoquinoline-3-carboxylic acid trifluoroacetate,
2-{3-[(benzylsulfonyl)amino]propyl}-8-quinolin-3-yl-4,5-dihydro-2H-pyrazolo[3,4-f]isoquinoline-3-carboxylic acid,
9-(aminomethyl)-5,6,9,10-tetrahydropyrazino[1′,2′:1,5]pyrazolo[3,4-f]isoquinolin-7(8H)-one trifluoroacetate,
2-(3-nitrophenyl)-5,6,8,9,10,11-hexahydro-7H-[1,4]diazepino[1′,2′:1,5]pyrazolo[3,4-f]isoquinolin-7-one,
2-(3-aminopropyl)-8-quinolin-3-yl-4,5-dihydro-2H-pyrazolo[3,4-f]isoquinoline-3-carboxamide hydrochloride,
2-(3-{[3-(4-chlorophenyl)propyl]amino}propyl)-4,5-dihydro-2H-pyrazolo[3,4-f]isoquinoline-3-carboxylic acid trifluoroacetate,
2-[3-(dimethylamino)phenyl]-5,6,8,9,10,11-hexahydro-7H-[1,4]diazepino[1′,2′:1,5]pyrazolo[3,4-f]isoquinolin-7-one,
2-(3-{[(4-chlorobenzyl)sulfonyl]amino}propyl)-8-quinolin-3-yl-4,5-dihydro-2H-pyrazolo[3,4-f]isoquinoline-3-carboxylic acid,
2-(3-{[2-(4-pyridin-3-ylphenyl)ethyl]amino}propyl)-4,5-dihydro-2H-pyrazolo[3,4-f]isoquinoline-3-carboxylic acid trifluoroacetate,
2-(3-{[2-(4-chlorophenyl)ethyl]amino}propyl)-4,5-dihydro-2H-pyrazolo[3,4-f]isoquinoline-3-carboxylic acid trifluoroacetate,
2-(3-{[2-(5-chlorothien-2-yl)ethyl]amino}propyl)-4,5-dihydro-2H-pyrazolo[3,4-f]isoquinoline-3-carboxylic acid trifluoroacetate,
2-(3-{[3-(4-cyanophenyl)propyl]amino}propyl)-4,5-dihydro-2H-pyrazolo[3,4-f]isoquinoline-3-carboxylic acid trifluoroacetate,
2-(3-{[3-(5-methyl-2-furyl)butyl]amino}propyl)-4,5-dihydro-2H-pyrazolo[3,4-f]isoquinoline-3-carboxylic acid trifluoroacetate,
2-[3-({2-[4-(1-benzothien-3-yl)phenyl]ethyl}amino)propyl]-4,5-dihydro-2H-pyrazolo[3,4-f]isoquinoline-3-carboxylic acid trifluoroacetate,
2-(3-ammoniopropyl)-3-carboxy-4,5-dihydro-2H-pyrazolo[3,4-f]isoquinolin-7-ium dichloride,
2-(4-methoxyphenyl)-5,6,9,10-tetrahydropyrazino[1′,2′:1,5]pyrazolo[3,4-f]isoquinolin-7(8H)-one,
2-(3-{[3-(4-acetylphenyl)propyl]amino}propyl)-4,5-dihydro-2H-pyrazolo[3,4-f]isoquinoline-3-carboxylic acid trifluoroacetate,
2-(3-aminopropyl)-4,5-dihydro-2H-pyrazolo[3,4-f]isoquinoline-3-carboxylic acid trifluoroacetate,
2-[3-({3-[4-(methylsulfonyl)phenyl]propyl}amino)propyl]-4,5-dihydro-2H-pyrazolo[3,4-f]isoquinoline-3-carboxylic acid trifluoroacetate,
2-(3-aminopropyl)-8-(2-methoxyphenyl)-4,5-dihydro-2H-pyrazolo[3,4-f]isoquinoline-3-carboxylic acid trifluoroacetate,
2-[3-({2-[3′-(aminomethyl)-1,1′-biphenyl-4-yl]ethyl}amino)propyl]-4,5-dihydro-2H-pyrazolo[3,4-f]isoquinoline-3-carboxylic acid trifluoroacetate,
2-(2-aminoethyl)-4,5-dihydro-2H-pyrazolo[3,4-f]isoquinoline-3-carboxylic acid dihydrochloride,
2-(3-{[2-(4-nitrophenyl)ethyl]amino}propyl)-4,5-dihydro-2H-pyrazolo[3,4-f]isoquinoline-3-carboxylic acid trifluoroacetate,
2-[3-({2-[2′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
9-(hydroxymethyl)-5,6,9,10-tetrahydropyrazino[1′,2′:1,5]pyrazolo[3,4-f]isoquinolin-7(8H)-one,
2-(3-{[2-(4-methylphenyl)ethyl]amino}propyl)-4,5-dihydro-2H-pyrazolo[3,4-f]isoquinoline-3-carboxylic acid trifluoroacetate,
9-{[(2-thien-2-ylethyl)amino]methyl}-5,6,9,10-tetrahydropyrazino[1′,2′:1,5]pyrazolo[3,4-f]isoquinolin-7(8H)-one,
2-(3-{[2-(4-ethoxyphenyl)ethyl]amino}propyl)-4,5-dihydro-2H-pyrazolo[3,4-f]isoquinoline-3-carboxylic acid trifluoroacetate,
2-(1,3-benzodioxol-5-yl)-5,6,8,9,10,11-hexahydro-7H-[1,4]diazepino[1′,2′:1,5]pyrazolo[3,4-f]isoquinolin-7-one,
2-(3-{[2-(4-methoxyphenyl)ethyl]amino}propyl)-4,5-dihydro-2H-pyrazolo[3,4-f]isoquinoline-3-carboxylic acid trifluoroacetate,
3-[3-(1H-tetraazol-5-yl)-4,5-dihydro-2H-pyrazolo[3,4-f]isoquinolin-2-yl]propan-1-amine hydrochloride,
2-(3-aminopropyl)-8-chloro-4,5-dihydro-2H-pyrazolo[3,4-f]isoquinoline-3-carboxylic acid trifluoroacetate,
2-(3-{[(1R,2S)-2-phenylcyclopropyl]amino}propyl)-4,5-dihydro-2H-pyrazolo[3,4-f]isoquinoline-3-carboxylic acid trifluoroacetate,
2-{3-[(3,3-diphenylpropyl)amino]propyl}-4,5-dihydro-2H-pyrazolo[3,4-f]isoquinoline-3-carboxylic acid trifluoroacetate,
2-(3-{[2-(3-bromo-4-methoxyphenyl)ethyl]amino}propyl)-4,5-dihydro-2H-pyrazolo[3,4-f]isoquinoline-3-carboxylic acid trifluoroacetate,
2-{3-[(4-phenylbutyl)amino]propyl}-4,5-dihydro-2H-pyrazolo[3,4-f]isoquinoline-3-carboxylic acid trifluoroacetate,
2-[3-(2,3-dihydro-1H-inden-2-ylamino)propyl]-4,5-dihydro-2H-pyrazolo[3,4-f]isoquinoline-3-carboxylic acid trifluoroacetate,
2-(2-naphthyl)-5,6,8,9,10,11-hexahydro-7H-[1,4]diazepino[1′,2′-: 1,5]pyrazolo[3,4-f]isoquinolin-7-one,
2-{3-[(3-phenylpropyl)amino]propyl}-4,5-dihydro-2H-pyrazolo[3,4-f]isoquinoline-3-carboxylic acid trifluoroacetate,
2-(3-{[2-(4-fluorophenyl)ethyl]amino}propyl)-4,5-dihydro-2H-pyrazolo[3,4-f]isoquinoline-3-carboxylic acid trifluoroacetate,
2-{3-[(2-thien-3-ylethyl)amino]propyl}-4,5-dihydro-2H-pyrazolo[3,4-f]isoquinoline-3-carboxylic acid dihydrochloride,
5,6,9,10-tetrahydropyrazino[1′,2′:1,5]pyrazolo[3,4-f]isoquinolin-7(8H)-one,
2-[3-(glycoloylamino)propyl]-8-quinolin-3-yl-4,5-dihydro-2H-pyrazolo[3,4-f]isoquinoline-3-carboxylic acid hydrochloride,
8-quinolin-3-yl-4,5-dihydro-2H-pyrazolo[3,4-f]isoquinoline-3-carboxylic acid,
2-(3-{[2-(4-ethylphenyl)ethyl]amino}propyl)-4,5-dihydro-2H-pyrazolo[3,4-f]isoquinoline-3-carboxylic acid trifluoroacetate,
2-(3-{[2-(2-chlorophenyl)ethyl]amino}propyl)-4,5-dihydro-2H-pyrazolo[3,4-f]isoquinoline-3-carboxylic acid trifluoroacetate,
2-{3-[(2-ethylbutyl)amino]propyl}-4,5-dihydro-2H-pyrazolo[3,4-f]isoquinoline-3-carboxylic acid trifluoroacetate,
8-quinolin-3-yl-4,5-dihydro-2H-pyrazolo[3,4-f]isoquinoline-3-carboxamide,
2-{3-[(2-pyridin-4-ylethyl)amino]propyl}-4,5-dihydro-2H-pyrazolo[3,4-f]isoquinoline-3-carboxylic acid trifluoroacetate,
2-(3-{[2-(4-chlorophenyl)propyl]amino}propyl)-4,5-dihydro-2H-pyrazolo[3,4-f]isoquinoline-3-carboxylic acid trifluoroacetate,
2-(3-{[2-(3-chlorophenyl)ethyl]amino}propyl)-4,5-dihydro-2H-pyrazolo[3,4-f]isoquinoline-3-carboxylic acid trifluoroacetate,
2-[3-(glycoloylamino)propyl]-4,5-dihydro-2H-pyrazolo[3,4-f]isoquinoline-3-carboxylic acid trifluoroacetate,
2-(3-{[2-(3,4-dimethoxyphenyl)ethyl]amino}propyl)-4,5-dihydro-2H-pyrazolo[3,4-f]isoquinoline-3-carboxylic acid trifluoroacetate,
2-(1-benzofuran-2-yl)-5,6,8,9,10,11-hexahydro-7H-[1,4]diazepino[1′,2′:1,5]pyrazolo[3,4-f]isoquinolin-7-one,
2-(3-{[4-(2-aminoethyl)phenyl]amino}propyl)-4,5-dihydro-2H-pyrazolo[3,4-f]isoquinoline-3-carboxylic acid trifluoroacetate,
2-(1-naphthyl)-5,6,9,10-tetrahydropyrazino[1′,2′:1,5]pyrazolo[3,4-f]isoquinolin-7(8H)-one,
8-(3-aminopropyl)-5,6,8,9,10,1,1-hexahydro-7H-[1,4]diazepino[1′,2′:1,5]pyrazolo[3,4-f]isoquinolin-7-one dihydrochloride,
2-anilino-5,6,9,10-tetrahydropyrazino[1′,2′:1,5]pyrazolo[3,4-f]isoquinolin-7(8H)-one,
2-(3-aminopropyl)-8-[2-(trifluoromethyl)phenyl]-4,5-dihydro-2H-pyrazolo[3,4-f]isoquinoline-3-carboxylic acid trifluoroacetate,
9-(azidomethyl)-5,6,9,10-tetrahydropyrazino[1′,2′:1,5]pyrazolo[3,4-f]isoquinolin-7(8H)-one,
9-({[2-(4-chlorophenyl)ethyl]amino}methyl)-5,6,9,10-tetrahydropyrazino[1′,2′:1,5]pyrazolo[3,4-f]isoquinolin-7(8H)-one,
2-phenyl-5,6,8,9,10,11-hexahydro-7H-[1,4]diazepino[1′,2′:1,5]pyrazolo[3,4-f]isoquinolin-7-one,
2-[3-(pentylamino)propyl]-4,5-dihydro-2H-pyrazolo[3,4-f]isoquinoline-3-carboxylic acid trifluoroacetate,
10-(2-aminoethyl)-5,6,9,10-tetrahydropyrazino[1′,2′:1,5]pyrazolo[3,4-f]isoquinolin-7(8H)-one,
2-[3-(allylamino)propyl]-4,5-dihydro-2H-pyrazolo[3,4-f]isoquinoline-3-carboxylic acid trifluoroacetate,
2-(4-aminobutyl)-4,5-dihydro-2H-pyrazolo[3,4-f]isoquinoline-3-carboxylic acid dihydrochloride,
2-(3-{[2-(4-aminophenyl)ethyl]amino}propyl)-4,5-dihydro-2H-pyrazolo[3,4-f]isoquinoline-3-carboxylic acid trifluoroacetate,
2-[(1E)-3,3-dimethylbut-1-enyl]-5,6,9,10-tetrahydropyrazino[1′,2′:1,5]pyrazolo[3,4-f]isoquinolin-7(8H)-one trifluoroacetate,
10-(nitromethyl)-5,6,9,10-tetrahydropyrazino[1′,2′:1,5]pyrazolo[3,4-f]isoquinolin-7(8H)-one,
3-carboxy-2-[3-(methylammonio)propyl]-4,5-dihydro-2H-pyrazolo[3,4-f]isoquinolin-7-ium dichloride,
2-[3-({[(4-butoxyphenyl)amino]carbonyl}amino)propyl]-4,5-dihydro-2H-pyrazolo[3,4-f]isoquinoline-3-carboxylic acid,
2-{3-[(2-pyridin-3-ylethyl)amino]propyl}-4,5-dihydro-2H-pyrazolo[3,4-f]isoquinoline-3-carboxylic acid trifluoroacetate,
2-{3-[(2-pyridin-2-ylethyl)amino]propyl}-4,5-dihydro-2H-pyrazolo[3,4-f]isoquinoline-3-carboxylic acid trifluoroacetate,
2-{3-[(cyclopropylmethyl)amino]propyl}-4,5-dihydro-2H-pyrazolo[3,4-f]isoquinoline-3-carboxylic acid trifluoroacetate,
2-{3-[(2-thien-2-ylpropyl)amino]propyl}-4,5-dihydro-2H-pyrazolo[3,4-f]isoquinoline-3-carboxylic acid trifluoroacetate,
2-methoxy-5,6,9,10-tetrahydropyrazino[1′,2′:1,5]pyrazolo[3,4-f]isoquinolin-7(8H)-one,
2-[3-(dimethylamino)phenyl]-5,6,9,10-tetrahydropyrazino[1′,2′:1,5]pyrazolo[3,4-f]isoquinolin-7(8H)-one,
2-(3-{[2-(1H-pyrrol-1-yl)ethyl]amino}propyl)-4,5-dihydro-2H-pyrazolo[3,4-f]isoquinoline-3-carboxylic acid trifluoroacetate,
2-[3-(benzyloxy)propyl]-8-quinolin-3-yl-4,5-dihydro-2H-pyrazolo[3,4-f]isoquinoline-3-carboxylic acid,
2-{3-[(4-butoxybenzyl)amino]propyl}-4,5-dihydro-2H-pyrazolo[3,4-f]isoquinoline-3-carboxylic acid trifluoroacetate,
2-(1,3-benzodioxol-5-yl)-5,6,9,10-tetrahydropyrazino[1′,2′:1,5]pyrazolo[3,4-f]isoquinolin-7(8H)-one,
2-[(E)-2-(2-fluorophenyl)ethenyl]-5,6,9,10-tetrahydropyrazino[1′,2′:1,5]pyrazolo[3,4-f]isoquinolin-7(8H)-one,
2-[(1E)-hex-1-enyl]-5,6,9,10-tetrahydropyrazino[1′,2′:1,5]pyrazolo[3,4-f]isoquinolin-7(8H)-one trifluoroacetate,
2-anilino-5,6,8,9,10,11-hexahydro-7H-[1,4]diazepino[1′,2′:1,5]pyrazolo[3,4-f]isoquinolin-7-one,
5,6,8,9,10,11-hexahydro-7H-[1,4]diazepino[1′,2′:1,5]pyrazolo[3,4-f]isoquinolin-7-one,
2-chloro-5,6,9,10-tetrahydropyrazino[1′,2′:1,]pyrazolo[3,4-f]isoquinolin-7(8H)-one,
2-[(E)-2-(4-methoxyphenyl)ethenyl]-5,6,9,10-tetrahydropyrazino[1′,2′:1,5]pyrazolo[3,4-f]isoquinolin-7(8H)-one,
2-(methylthio)-5,6,9,10-tetrahydropyrazino[1′,2′:1,5]pyrazolo[3,4-f]isoquinolin-7(8H)-one,
2-{3-[(2-furylmethyl)amino]propyl}-4,5-dihydro-2H-pyrazolo[3,4-f]isoquinoline-3-carboxylic acid trifluoroacetate,
2-azepan-1-yl-5,6,9,10-tetrahydropyrazino[1′,2′:1,5]pyrazolo[3,4-f]isoquinolin-7(8H)-one trifluoroacetate,
2-(3,6-dihydropyridin-1 (2H)-yl)-5,6,9,10-tetrahydropyrazino[1′,2′:1,5]pyrazolo[3,4-f]isoquinolin-7(8H)-one trifluoroacetate,
9-methyl-5,6,9,10-tetrahydropyrazino[1′,2′:1,5]pyrazolo[3,4-f]isoquinolin-7(8H)-one,
2-(piperidin-3-ylmethyl)-4,5-dihydro-2H-pyrazolo[3,4-f]isoquinoline-3-carboxylic acid hydrochloride,
9-(chloromethyl)-5,6,9,10-tetrahydropyrazino[1′,2′:1,5]pyrazolo[3,4-f]isoquinolin-7(8H)-one,
2-[(4-methoxybenzyl)amino]-5,6,9,10-tetrahydropyrazino[1′,2′:1,5]pyrazolo[3,4-f]isoquinolin-7(8H)-one trifluoroacetate,
2-(3-{[2-(1H-imidazol-4-yl)ethyl]amino}propyl)-4,5-dihydro-2H-pyrazolo[3,4-f]isoquinoline-3-carboxylic acid trifluoroacetate,
2-(benzylamino)-5,6,9,10-tetrahydropyrazino[1′,2′:1,5]pyrazolo[3,4-f]isoquinolin-7(8H)-one trifluoroacetate,
2-(methylthio)-5,6,8,9,10,11-hexahydro-7H-[1,4]diazepino[1′,2′:1,5]pyrazolo[3,4-f]isoquinolin-7-one,
2-{3-[(2-chlorobenzyl)amino]propyl}-4,5-dihydro-2H-pyrazolo[3,4-f]isoquinoline-3-carboxylic acid trifluoroacetate,
2-[3-(benzylamino)propyl]-4,5-dihydro-2H-pyrazolo[3,4-f]isoquinoline-3-carboxylic acid trifluoroacetate,
2-[3-({[(4-methoxyphenyl)amino]carbonyl}amino)propyl]-4,5-dihydro-2H-pyrazolo[3,4-f]isoquinoline-3-carboxylic acid,
2-{3-[(2-phenylethyl)amino]propyl}-4,5-dihydro-2H-pyrazolo[3,4-f]isoquinoline-3-carboxylic acid trifluoroacetate,
2-{3-[(thien-2-ylmethyl)amino]propyl}-4,5-dihydro-2H-pyrazolo[3,4-f]isoquinoline-3-carboxylic acid trifluoroacetate,
2-benzyl-5,6,9,10-tetrahydropyrazino[1′,2′:1,5]pyrazolo[3,4-f]isoquinolin-7(8H)-one,
5,6,8,9,10,11-hexahydro-7H-[1,4]diazepino[1′,2′:1,5]pyrazolo[3,4-f]isoquinolin-7-one,
2-{3-[(4-chlorobenzyl)amino]propyl}-4,5-dihydro-2H-pyrazolo[3,4-f]isoquinoline-3-carboxylic acid trifluoroacetate,
2-{3-[(2-phenylpropyl)amino]propyl}-4,5-dihydro-2H-pyrazolo[3,4-f]isoquinoline-3-carboxylic acid trifluoroacetate,
7-oxo-5,6,7,8,9,10-hexahydropyrazino[1′,2′:1,5]pyrazolo[3,4-f]isoquinoline-9-carboxamide trifluoroacetate,
2-(3-hydroxypropyl)-4,5-dihydro-2H-pyrazolo[3,4-f]isoquinoline-3-carboxylic acid,
2-(1,3-dihydro-2H-isoindol-2-yl)-5,6,9,10-tetrahydropyrazino[1′,2′:1,5]pyrazolo[3,4-f]isoquinolin-7(8H)-one trifluoroacetate,
2-{3-[(4-aminophenyl)amino]propyl}-4,5-dihydro-2H-pyrazolo[3,4-f]isoquinoline-3-carboxylic acid trifluoroacetate,
2-(4-hydroxypiperidin-1-yl)-5,6,9,10-tetrahydropyrazino[1′,2′:1,5]pyrazolo[3,4-f]isoquinolin-7(8H)-one trifluoroacetate,
2-{3-[(1H-imidazol-5-ylmethyl)amino]propyl}-4,5-dihydro-2H-pyrazolo[3,4-f]isoquinoline-3-carboxylic acid trifluoroacetate,
2-{3-[(3-chlorobenzyl)amino]propyl}-4,5-dihydro-2H-pyrazolo[3,4-f]isoquinoline-3-carboxylic acid trifluoroacetate,
2-{3-[(pyridin-3-ylcarbonyl)amino]propyl}-4,5-dihydro-2H-pyrazolo[3,4-f]isoquinoline-3-carboxylic acid trifluoroacetate,
4,5-dihydro-2H-pyrazolo[3,4-f]isoquinoline-3-carboxamide,
2-[3-(cyclopentylamino)propyl]-4,5-dihydro-2H-pyrazolo[3,4-f]isoquinoline-3-carboxylic acid dihydrochloride,
2-(3-{[2-(1H-indol-3-yl)ethyl]amino}propyl)-4,5-dihydro-2H-pyrazolo[3,4-f]isoquinoline-3-carboxylic acid,
10-(3-aminopropyl)-5,6,9,10-tetrahydropyrazino[1′,2′:1,5]pyrazolo[3,4-f]isoquinolin-7(8H)-one,
9-(1,2-dihydroxyethyl)-5,6,9,10-tetrahydropyrazino[1′,2′:1,5]pyrazolo[3,4-f]isoquinolin-7(8H)-one,
2-morpholin-4-yl-5,6,9,10-tetrahydropyrazino[1′,2′:1,5]pyrazolo[3,4-f]isoquinolin-7(8H)-one trifluoroacetate,
ethyl 2-(2-aminoethyl)-4,5-dihydro-2H-pyrazolo[3,4-f]isoquinoline-3-carboxylate dihydrochloride,
2-allyl-4,5-dihydro-2H-pyrazolo[3,4-f]isoquinoline-3-carboxylic acid,
2-quinolin-8-yl-5,6,9,10-tetrahydropyrazino[1′,2′:1,5]pyrazolo[3,4-f]isoquinolin-7(8H)-one,
lithium 2-[3-(2,3-dihydro-1H-inden-2-ylamino)-2-hydroxypropyl]-4,5-dihydro-2H-pyrazolo[3,4-f]isoquinoline-3-carboxylate,
2-(2-hydroxyethyl)-4,5-dihydro-2H-pyrazolo[3,4-f]isoquinoline-3-carboxylic acid,
2-{3-[(2-pyrrolidin-1-ylethyl)amino]propyl}-4,5-dihydro-2H-pyrazolo[3,4-f]isoquinoline-3-carboxylic acid trifluoroacetate,
2-(3,4-dihydroisoquinolin-2(1H)-yl)-5,6,9,10-tetrahydropyrazino[1′,2′:1,5]pyrazolo[3,4-f]isoquinolin-7(8H)-one trifluoroacetate,
2-(3-{[(benzylamino)carbonyl]amino}propyl)-4,5-dihydro-2H-pyrazolo[3,4-f]isoquinoline-3-carboxylic acid,
2-[3-(dimethylamino)propyl]-4,5-dihydro-2H-pyrazolo[3,4-f]isoquinoline-3-carboxylic acid trifluoroacetate,
10-(3-{([2-(4-chlorophenyl)ethyl]amino}propyl)-5,6,9,10-tetrahydropyrazino[1′,2′:1,5]pyrazolo[3,4-f]isoquinolin-7(8H)-one,
2-(3-aminopropyl)-6-chloro-4,5-dihydro-2H-pyrazolo[3,4-f]isoquinoline-3-carboxylic acid trifluoroacetate,
methyl 7-oxo-5,6,7,8,9,10-hexahydropyrazino[1′,2′:1,5]pyrazolo[3,4-f]isoquinoline-9-carboxylate,
2-[2-(glycoloylamino)ethyl]-45-dihydro-2H-pyrazolo[3,4-f]isoquinoline-3-carboxylic acid hydrochloride,
2-[2-(isopropylamino)ethyl]-4,5-dihydro-2H-pyrazolo[3,4-f]isoquinoline-3-carboxylic acid hydrochloride,
2-(3-cyanopropyl)-4,5-dihydro-2H-pyrazolo[3,4-f]isoquinoline-3-carboxylic acid,
2-[3-({[(3-cyanophenyl)amino]carbonyl}amino)propyl]-4,5-dihydro-2H-pyrazolo[3,4-f]isoquinoline-3-carboxylic acid,
9-(aminomethyl)-5,6,9,10-tetrahydro-7H-[1,4]oxazino[4′,3′:1,5]pyrazolo[3,4-t]isoquinolin-7-one trifluoroacetate,
4-methyl-5,6,8,9,10,11-hexahydro-7H-[1,4]diazepino[1′,2′:1,5]pyrazolo[3,4-f]isoquinolin-7-one trifluoroacetate,
2-propyl-4,5-dihydro-2H-pyrazolo[3,4-f]isoquinoline-3-carboxylic acid,
2-(3-bromopropyl)-4,5-dihydro-2H-pyrazolo[3,4-f]isoquinoline-3-carboxylic acid trifluoroacetate,
2-pyrrolidin-1-yl-5,6,9,10-tetrahydropyrazino[1′,2′:1,5]pyrazolo[3,4-f]isoquinolin-7(8H)-one,
2-[3-(isopropylamino)propyl]-4,5-dihydro-2H-pyrazolo[3,4-f]isoquinoline-3-carboxylic acid hydrochloride,
2-(3-amino-3-carboxypropyl)-4,5-dihydro-2H-pyrazolo[3,4-f]isoquinoline-3-carboxylic acid trifluoroacetate,
2-{3-[(aminocarbonyl)amino]propyl}-4,5-dihydro-2H-pyrazolo[3,4-f]isoquinoline-3-carboxylic acid,
9-{[(4-chlorobenzyl)amino]methyl}-5,6,9,10-tetrahydropyrazino[1′,2′:1,5]pyrazolo[3,4-f]isoquinolin-7(8H)-one,
4-chloro-5,6,9,10-tetrahydropyrazino[1′,2′:1,5]pyrazolo[3,4-f]isoquinolin-7(8H)-one,
7-oxo-2-quinolin-3-yl-5,6,7,8,9,10-hexahydropyrazino[1′,2′:1,5]pyrazolo[3,4-f]isoquinoline-9-carboxamide,
2-{3-[(methylsulfonyl)amino]propyl}-4,5-dihydro-2H-pyrazolo[3,4-f]isoquinoline-3-carboxylic acid trifluoroacetate,
lithium 2-{4-[(2-ethylbutyl)amino]butyl}-4,5-dihydro-2H-pyrazolo[3,4-f]isoquinoline-3-carboxylate,
2-(3-{[(4-methoxyphenyl)acetyl]amino}propyl)-4,5-dihydro-2H-pyrazolo[3,4-f]isoquinoline-3-carboxylic acid trifluoroacetate,
7-oxo-6,7,8,9,10,11-hexahydro-5H-[1,4]diazepino[1′,2′:1,5]pyrazolo[3,4-f]isoquinoline-9-carbonitrile,
2-{3-[(isopropylsulfonyl)amino]propyl}-4,5-dihydro-2H-pyrazolo[3,4-f]isoquinoline-3-carboxylic acid trifluoroacetate,
3-[3-(chloromethyl)-4,5-dihydro-2H-pyrazolo[3,4-f]isoquinolin-2-yl]propan-1-amine hydrochloride,
2-(3,3-dimethylbutyl)-5,6,9,10-tetrahydropyrazino[1′,2′:1,5]pyrazolo[3,4-f]isoquinolin-7(8H)-one trifluoroacetate,
2-thiomorpholin-4-yl-5,6,9,10-tetrahydropyrazino[1′,2′:1,5]pyrazolo[3,4-f]isoquinolin-7(8H)-one trifluoroacetate,
ethyl 2-(2-aminoethyl)-8-chloro-4,5-dihydro-2H-pyrazolo[3,4-f]isoquinoline-3-carboxylate,
2-[4-(benzyloxy)phenyl]-5,6,9,10-tetrahydropyrazino[1′,2′:1,5]pyrazolo[3,4-f]isoquinolin-7(8H)-one,
lithium 2-{2-hydroxy-3-[(thien-2-ylmethyl)amino]propyl}-4,5-dihydro-2H-pyrazolo[3,4-f]isoquinoline-3-carboxylate,
2-(3-amino-3-cyanopropyl)-4,5-dihydro-2H-pyrazolo[3,4-f]isoquinoline-3-carboxylic acid trifluoroacetate,
2-[3-(pyridin-3-ylamino)propyl]-4,5-dihydro-2H-pyrazolo[3,4-f]isoquinoline-3-carboxylic acid trifluoroacetate,
2-[3-({[(ethoxycarbonyl)amino]carbonyl}amino)propyl]-4,5-dihydro-2H-pyrazolo[3,4-f]isoquinoline-3-carboxylic acid trifluoroacetate,
2-{3-[4-(aminocarbonyl)piperidin-1-yl]propyl}-4,5-dihydro-2H-pyrazolo[3,4-f]isoquinoline-3-carboxylic acid trifluoroacetate,
2-(3-aminopropyl)-7-hydroxy-8-(3-nitrophenyl)-4,5-dihydro-2H-benzo[g]indazole-3-carboxylic acid trifluoroacetate,
2-(2-aminoethyl)-7-hydroxy-8-(3-nitrophenyl)-4,5-dihydro-2H-benzo[g]indazole-3-carboxylic acid trifluoroacetate,
3-hydroxy-2-(3-nitrophenyl)-5,6,8,9,10,11-hexahydro-7H-benzo[g][1,4]diazepino[1,2-b]indazol-7-one trifluoroacetate,
2-(3-aminopropyl)-7-hydroxy-4,5-dihydro-2H-benzo[g]indazole-3-carboxylic acid dihydrochloride,
2-(2-aminoethyl)-8-bromo-7-hydroxy-4,5-dihydro-2H-benzo[g]indazole-3-carboxylic acid trifluoroacetate,
2-(3-aminopropyl)-8-[2-(4-chlorophenyl)ethyl]-7-hydroxy-4,5-dihydro-2H-benzo[g]indazole-3-carboxylic acid trifluoroacetate,
3-hydroxy-2-(3-nitrophenyl)-5,6,9,10-tetrahydrobenzo[g]pyrazino[1,2-b]indazol-7(8H)-one hydrobromide,
2-(3-aminopropyl)-8-bromo-7-hydroxy-4,5-dihydro-2H-benzo[g]indazole-3-carboxylic acid hydrobromide,
2-(3-aminopropyl)-7-hydroxy-8-(4-nitrophenyl)-4,5-dihydro-2H-benzo[g]indazole-3-carboxylic acid trifluoroacetate,
2-(3-aminopropyl)-8-(3-cyanophenyl)-7-hydroxy-4,5-dihydro-2H-benzo[g]indazole-3-carboxylic acid trifluoroacetate,
2-(3-aminopropyl)-7-hydroxy-8-[3-(trifluoromethyl)phenyl]-4,5-dihydro-2H-benzo[g]indazole-3-carboxylic acid trifluoroacetate,
2-(3-aminopropyl)-7-hydroxy-8-(3,3,3-trifluoropropyl)-4,5-dihydro-2H-benzo[g]indazole-3-carboxylic acid trifluoroacetate,
2-(3-aminopropyl)-8-(3-chlorophenyl)-7-hydroxy-4,5-dihydro-2H-benzo[g]indazole-3-carboxylic acid trifluoroacetate,
2-(3-aminopropyl)-7-hydroxy-5-methyl-4,5-dihydro-2H-benzo[g]indazole-3-carboxylic acid trifluoroacetate,
2-(3-aminopropyl)-8-(4-chlorophenyl)-7-hydroxy-4,5-dihydro-2H-benzo[g]indazole-3-carboxylic acid trifluoroacetate,
2-(3-aminopropyl)-7-hydroxy-8-[3-(methylsulfonyl)phenyl]-4,5-dihydro-2H-benzo[g]indazole-3-carboxylic acid trifluoroacetate,
2-(3-aminopropyl)-8-(3,4-difluorophenyl)-7-hydroxy-4,5-dihydro-2H-benzo[g]indazole-3-carboxylic acid trifluoroacetate,
3-hydroxy-5,6,8,9,10,11-hexahydro-7H-benzo[g][1,4]diazepino[1,2-b]indazol-7-one,
8-(3-aminophenyl)-2-(3-aminopropyl)-7-hydroxy-4,5-dihydro-2H-benzo[g]indazole-3-carboxylic acid trifluoroacetate,
6-bromo-7-hydroxy-4,5-dihydro-2H-benzo[g]indazole-3-carboxamide,
2-(3-aminopropyl)-7-hydroxy-8-[4-(methylamino)phenyl]-4,5-dihydro-2H-benzo[g]indazole-3-carboxylic acid trifluoroacetate,
2-(3-aminopropyl)-8-(4-tert-butylphenyl)-7-hydroxy-4,5-dihydro-2H-benzo[g]indazole-3-carboxylic acid trifluoroacetate,
2-(3-aminopropyl)-7-hydroxy-8-pyridin-4-yl-4,5-dihydro-2H-benzo[g]indazole-3-carboxylic acid trifluoroacetate,
7-hydroxy-4,5-dihydro-2H-benzo[g]indazole-3-carboxylic acid,
2-(3-aminopropyl)-7-hydroxy-8-isobutyl-4,5-dihydro-2H-benzo[g]indazole-3-carboxylic acid trifluoroacetate,
2-(3-aminopropyl)-8-(4-carboxy-3,3-dimethylbutyl)-7-hydroxy-4,5-dihydro-2H-benzo[g]indazole-3-carboxylic acid trifluoroacetate,
8-amino-2-(3-aminopropyl)-7-hydroxy-4,5-dihydro-2H-benzo[g]indazole-3-carboxylic acid,
7,8-dihydroxy-4,5-dihydro-2H-benzo[g]indazole-3-carboxylic acid,
2-(3-aminopropyl)-8-benzyl-7-hydroxy-4,5-dihydro-2H-benzo[g]indazole-3-carboxylic acid trifluoroacetate,
2-(3-aminopropyl)-8-(2-chlorophenyl)-7-hydroxy-4,5-dihydro-2H-benzo[g]indazole-3-carboxylic acid trifluoroacetate,
2-allyl-7-hydroxy-4,5-dihydro-2H-benzo[g]indazole-3-carboxylic acid,
2-{3-((tert-butoxycarbonyl)amino]propyl}-7-methoxy-8-(4-nitrophenyl)-4,5-dihydro-2H-benzo[g]indazole-3-carboxylic acid,
7-hydroxy-2-methyl-4,5-dihydro-2H-benzo[g]indazole-3-carboxylic acid,
2-(3-aminopropyl)-8-(sec-butylamino)-7-hydroxy-4,5-dihydro-2H-benzo[g]indazole-3-carboxylic acid, and
2-(3-aminopropyl)-8-(3-carboxyphenyl)-7-hydroxy-4,5-dihydro-2H-benzo[g]indazole-3-carboxylic acid trifluoroacetate.

19. The cyclic pyrazole MK-2 inhibitor according to claim 1, wherein the MK-2 inhibitor has an MK-2 IC₅₀ of less than 20 and is selected from the group consisting of:
2-(3-aminopropyl)-8-(methylthio)-2,4,5,6-tetrahydropyrazolo[3,4-e]indazole-3-carboxylic acid dihydrochloride,
2-(3-aminopropyl)-2,4,5,6-tetrahydropyrazolo[3,4-e]indazole-3-carboxylic acid hydrochloride,
2-(3-{[2-(4-bromophenyl)ethyl]amino}propyl)-2,4,5,6-tetrahydropyrazolo[3,4-e]indazole-3-carboxylic acid hydrochloride,
2-(2-aminoethyl)-2,4,5,6-tetrahydropyrazolo[3,4-e]indazole-3-carboxylic acid hydrochloride,
8-(allylthio)-2-(3-aminopropyl)-2,4,5,6-tetrahydropyrazolo[3,4-e]indazole-3-carboxylic acid dihydrochloride,
2-(3-aminopropyl)-8-(benzylthio)-2,4,5,6-tetrahydropyrazolo[3,4-e]indazole-3-carboxylic acid dihydrochloride,
2-{3-[(2-thien-2-ylethyl)amino]propyl}-2,4,5,6-tetrahydropyrazolo[3,4-e]indazole-3-carboxylic acid,
2-{3-[(2-thien-3-ylethyl)amino]propyl}-2,4,5,6-tetrahydropyrazolo[3,4-e]indazole-3-carboxylic acid hydrochloride,
ethyl 2-(3-{[2-(4-bromophenyl)ethyl]amino}propyl)-2,4,5,6-tetrahydropyrazolo[3,4-e]indazole-3-carboxylate,
2-(4-aminobutyl)-2,4,5,6-tetrahydropyrazolo[3,4-e]indazole-3-carboxylic acid dihydrochloride,
1-(methylthio)-4,5,7,8,9,10-hexahydro[1,4]diazepino[1,2-b]pyrazolo[3,4-g]indazol-6(3H)-one,
1-(allylthio)-4,5,7,8,9,10-hexahydro[1,4]diazepino[1,2-b]pyrazolo[3,4-g]indazol-6(3H)-one,
4,5,7,8,9,10-hexahydro[1,4]diazepino[1,2-b]pyrazolo[3,4-g]indazol-6(3H)-one,
2-(3-aminopropyl)-8-quinolin-3-yl-4,5-dihydro-2H-pyrazolo[4,3-h]quinazoline-3-carboxylic acid dihydrochloride,
2-(3-aminopropyl)-8-(1,3-benzodioxol-5-yl)-4,5-dihydro-2H-pyrazolo[4,3-h]quinazoline-3-carboxylic acid hydrochloride,
2-(3-aminopropyl)-8-phenyl-4,5-dihydro-2H-pyrazolo[4,3-h]quinazoline-3-carboxylic acid hydrochloride,
2-quinolin-3-yl-5,6,8,9,10,11-hexahydro-7H-[1,4]diazepino[1′,2′:1,5]pyrazolo[4,3-h]quinazolin-7-one,
2-pyridin-3-yl-5,6,8,9,10,11-hexahydro-7H-[1,4]diazepino[1′,2′:1,5]pyrazolo[4,3-h]quinazolin-7-one,
8-quinolin-3-yl-2-[3-(tritylamino)propyl]-4,5-dihydro-2H-pyrazolo[4,3-h]quinazoline-3-carboxylic acid hydrochloride,
2-(1,3-benzodioxol-5-yl)-5,6,8,9,10,11-hexahydro-7H-[1,4]diazepino[1′,2′:1,5]pyrazolo[4,3-h]quinazolin-7-one,
2-(4-methoxyphenyl)-5,6,8,9,10,11-hexahydro-7H-[1,4]diazepino[1′,2′:1,5]pyrazolo[4,3-h]quinazolin-7-one,
2-pyridin-4-yl-5,6,8,9,10,11-hexahydro-7H-[1,4]diazepino[1′,2′:1,5]pyrazolo[4,3-h]quinazolin-7-one hydrochloride,
2-(3-aminopropyl)-8-quinolin-3-yl-4,5-dihydro-2H-pyrazolo[3,4-f]isoquinoline-3-carboxylic acid trifluoroacetate,
2-(3-aminopropyl)-8-(3-nitrophenyl)-4,5-dihydro-2H-pyrazolo[3,4-f]isoquinoline-3-carboxylic acid trifluoroacetate,
2-(3-aminopropyl)-8-(4-hydroxyphenyl)-4,5-dihydro-2H-pyrazolo[3,4-f]isoquinoline-3-carboxylic acid hydrobromide,
2-(3-aminopropyl)-8-(3-hydroxyphenyl)-4,5-dihydro-2H-pyrazolo[3,4-f]isoquinoline-3-carboxylic acid hydrobromide,
2-(3-aminopropyl)-8-(2-naphthyl)-4,5-dihydro-2H-pyrazolo[3,4-f]isoquinoline-3-carboxylic acid trifluoroacetate,
2-(3-aminopropyl)-8-(3,5-difluorophenyl)-4,5-dihydro-2H-pyrazolo[3,4-f]isoquinoline-3-carboxylic acid trifluoroacetate,
2-(3-aminopropyl)-8-(1,3-benzodioxol-5-yl)-4,5-dihydro-2H-pyrazolo[3,4-f]isoquinoline-3-carboxylic acid trifluoroacetate,
2-(3-aminopropyl)-8-(3-cyanophenyl)-4,5-dihydro-2H-pyrazolo[3,4-f]isoquinoline-3-carboxylic acid trifluoroacetate,
9-(hydroxymethyl)-2-quinolin-3-yl-5,6,9,10-tetrahydropyrazino[1′,2′:1,5]pyrazolo[3,4-f]isoquinolin-7(8H)-one trifluoroacetate,
2-(3-aminopropyl)-8-(4-methoxyphenyl)-4,5-dihydro-2H-pyrazolo[3,4-f]isoquinoline-3-carboxylic acid trifluoroacetate,
2-(3-aminopropyl)-8-[3-(methylsulfonyl)phenyl]-4,5-dihydro-2H-pyrazolo[3,4-f]isoquinoline-3-carboxylic acid trifluoroacetate,
2-(3-aminopropyl)-8-[3-(methylsulfonyl)phenyl]-4,5-dihydro-2H-pyrazolo[3,4-f]isoquinoline-3-carboxylic acid trifluoroacetate,
2-(1H-imidazol-1-yl)-5,6,9,10-tetrahydropyrazino[1′,2′:1,5]pyrazolo[3,4-f]isoquinolin-7(8H)-one trifluoroacetate,
2-(3-aminopropyl)-8-(3-methoxyphenyl)-4,5-dihydro-2H-pyrazolo[3,4-f]isoquinoline-3-carboxylic acid trifluoroacetate,
2-(3-aminopropyl)-8-[4-(trifluoromethyl)phenyl]-4,5-dihydro-2H-pyrazolo[3,4-f]isoquinoline-3-carboxylic acid trifluoroacetate,
2-(3-aminopropyl)-8-anilino-4,5-dihydro-2H-pyrazolo[3,4-f]isoquinoline-3-carboxylic acid,
2-(3-aminopropyl)-8-(3,4-difluorophenyl)-4,5-dihydro-2H-pyrazolo[3,4-f]isoquinoline-3-carboxylic acid trifluoroacetate,
2-(3-{[2-(4′-carboxy-1,1′-biphenyl-4-yl)ethyl]amino}propyl)-4,5-dihydro-2H-pyrazolo[3,4-f]isoquinoline-3-carboxylic acid trifluoroacetate,
2-(4-hydroxyphenyl)-5,6,9,10-tetrahydropyrazino[1′,2′:1,5]pyrazolo[3,4-f]isoquinolin-7(8H)-one trifluoroacetate,
2-propyl-8-quinolin-3-yl-4,5-dihydro-2H-pyrazolo[3,4-f]isoquinoline-3-carboxylic acid,
2-[3-({2-[3′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
2-(3-aminopropyl)-8-(4-tert-butylphenyl)-4,5-dihydro-2H-pyrazolo[3,4-f]isoquinoline-3-carboxylic acid trifluoroacetate,
2-[3-({2-(4-(3-furyl)phenyl]ethyl}amino)propyl]-4,5-dihydro-2H-pyrazolo[3,4-f]isoquinoline-3-carboxylic acid trifluoroacetate,
2-(3-{[2-(3′-chloro-1,1′-biphenyl-4-yl)ethyl]amino}propyl)-4,5-dihydro-2H-pyrazolo[3,4-f]isoquinoline-3-carboxylic acid trifluoroacetate,
2-(4-methoxyphenyl)-5,6,8,9,10,11-hexahydro-7H-[1,4]diazepino[1′,2′:1,5]pyrazolo[3,4-f]isoquinolin-7-one,
2-[3-({2-[4′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
2-(3-hydroxyphenyl)-5,6,9,10-tetrahydropyrazino[1′,2′:1,5]pyrazolo[3,4-f]isoquinolin-7(8H)-one,
2-(3-aminopropyl)-N-hydroxy-8-quinolin-3-yl-4,5-dihydro-2H-pyrazolo[3,4-f]isoquinoline-3-carboxamide hydrochloride,
2-[(E)-2-(4-hydroxyphenyl)ethenyl]-5,6,9,10-tetrahydropyrazino[1′,2′:1,5]pyrazolo[3,4-f]isoquinolin-7(8H)-one trifluoroacetate,
2-quinolin-3-yl-5,6,8,9,10,11-hexahydro-7H-[1,4]diazepino[1′,2′:1,5]pyrazolo[3,4-f]isoquinolin-7-one,
2-(4-hydroxyphenyl)-5,6,8,9,10,11-hexahydro-7H-[1,4]diazepino[1′,2′:1,5]pyrazolo[3,4-f]isoquinolin-7-one,
2-(3-{[2-(2′-chloro-1,1′-biphenyl-4-yl)ethyl]amino}propyl)-4,5-dihydro-2H-pyrazolo[3,4-f]isoquinoline-3-carboxylic acid trifluoroacetate,
2-(3-{[2-(4′-tert-butyl-1,1′-biphenyl-4-yl)ethyl]amino}propyl)-4,5-dihydro-2H-pyrazolo[3,4-f]isoquinoline-3-carboxylic acid trifluoroacetate,
2-(3-{[2-(3,4-dichlorophenyl)ethyl]amino}propyl)-4,5-dihydro-2H-pyrazolo[3,4-f]isoquinoline-3-carboxylic acid trifluoroacetate,
2-(3-{[3-(3-chlorophenyl)propyl]amino}propyl)-4,5-dihydro-2H-pyrazolo[3,4-f]isoquinoline-3-carboxylic acid trifluoroacetate,
2-[(E)-2-phenylethenyl]-5,6,9,10-tetrahydropyrazino[1′,2′:1,5]pyrazolo[3,4-f]isoquinolin-7(8H)-one trifluoroacetate,
2-(3-{[2-(4-pyridin-4-ylphenyl)ethyl]amino}propyl)-4,5-dihydro-2H-pyrazolo[3,4-f]isoquinoline-3-carboxylic acid trifluoroacetate,
2-(3-{[3-(4-bromophenyl)propyl]amino}propyl)-4,5-dihydro-2H-pyrazolo[3,4-f]isoquinoline-3-carboxylic acid trifluoroacetate,
2-(3-{[3-(4-tert-butylphenyl)propyl]amino}propyl)-4,5-dihydro-2H-pyrazolo[3,4-f]isoquinoline-3-carboxylic acid trifluoroacetate,
2-(3-{[2-(3′-isopropyl-1,1′-biphenyl-4-yl)ethyl]amino}propyl)-45-dihydro-2H-pyrazolo[3,4-f]isoquinoline-3-carboxylic acid trifluoroacetate,
2-{3-[(2-thien-2-ylethyl)amino]propyl}-4,5-dihydro-2H-pyrazolo[3,4-f]isoquinoline-3-carboxylic acid trifluoroacetate,
2-[4-(dimethylamino)phenyl]-5,6,9,10-tetrahydropyrazino[1′,2′:1,5]pyrazolo[3,4-f]isoquinolin-7(8H)-one,
2-(3-{[2-(1,1′-biphenyl-4-yl)ethyl]amino}propyl)-4,5-dihydro-2H-pyrazolo[3,4-f]isoquinoline-3-carboxylic acid trifluoroacetate,
2-(3-methoxyphenyl)-5,6,9,10-tetrahydropyrazino[1′,2′:1,5]pyrazolo[3,4-f]isoquinolin-7(8H)-one,
2-(3-{[2-(4-bromophenyl)ethyl]amino}propyl)-4,5-dihydro-2H-pyrazolo[3,4-f]isoquinoline-3-carboxylic acid trifluoroacetate,
2-(3-{[2-(2,4-dichlorophenyl)ethyl]amino}propyl)-4,5-dihydro-2H-pyrazolo[3,4-f]isoquinoline-3-carboxylic acid trifluoroacetate,
2-{3-[(benzylsulfonyl)amino]propyl}-8-quinolin-3-yl-4,5-dihydro-2H-pyrazolo[3,4-f]isoquinoline-3-carboxylic acid,
9-(aminomethyl)-5,6,9,10-tetrahydropyrazino[1′,2′:1,5]pyrazolo[3,4-f]isoquinolin-7(8H)-one trifluoroacetate,
2-(3-nitrophenyl)-5,6,8,9,10,11-hexahydro-7H-[1,4]diazepino[1′,2′:1,5]pyrazolo[3,4-f]isoquinolin-7-one,
2-(3-aminopropyl)-8-quinolin-3-yl-4,5-dihydro-2H-pyrazolo[3,4-f]isoquinoline-3-carboxamide hydrochloride,
2-(3-{[3-(4-chlorophenyl)propyl]amino}propyl)-4,5-dihydro-2H-pyrazolo[3,4-f]isoquinoline-3-carboxylic acid trifluoroacetate,
2-[3-(dimethylamino)phenyl]-5,6,8,9,10,11-hexahydro-7H-[1,4]diazepino[1′,2′:1,5]pyrazolo[3,4-f]isoquinolin-7-one,
2-(3-{[(4-chlorobenzyl)sulfonyl]amino}propyl)-8-quinolin-3-yl-4,5-dihydro-2H-pyrazolo[3,4-f]isoquinoline-3-carboxylic acid,
2-(3-{[2-(4-pyridin-3-ylphenyl)ethyl]amino}propyl)-4,5-dihydro-2H-pyrazolo[3,4-f]isoquinoline-3-carboxylic acid trifluoroacetate,
2-(3-{[2-(4-chlorophenyl)ethyl]amino}propyl)-4,5-dihydro-2H-pyrazolo[3,4-f]isoquinoline-3-carboxylic acid trifluoroacetate,
2-(3-{[2-(5-chlorothien-2-yl)ethyl]amino}propyl)-4,5-dihydro-2H-pyrazolo[3,4-f]isoquinoline-3-carboxylic acid trifluoroacetate,
2-(3-{[3-(4-cyanophenyl)propyl]amino}propyl)-4,5-dihydro-2H-pyrazolo[3,4-f]isoquinoline-3-carboxylic acid trifluoroacetate,
2-(3-{[3-(5-methyl-2-furyl)butyl]amino}propyl)-4,5-dihydro-2H-pyrazolo[3,4-f]isoquinoline-3-carboxylic acid trifluoroacetate,
2-[3-({2-[4-(1-benzothien-3-yl)phenyl]ethyl}amino)propyl]-4,5-dihydro-2H-pyrazolo[3,4-f]isoquinoline-3-carboxylic acid trifluoroacetate,
2-(3-ammoniopropyl)-3-carboxy-4,5-dihydro-2H-pyrazolo[3,4-f]isoquinolin-7-ium dichloride,
2-(4-methoxyphenyl)-5,6,9,10-tetrahydropyrazino[1′,2′:1,5]pyrazolo[3,4-f]isoquinolin-7(8H)-one,
2-(3-{[3-(4-acetylphenyl)propyl]amino}propyl)-4,5-dihydro-2H-pyrazolo[3,4-f]isoquinoline-3-carboxylic acid trifluoroacetate,
2-(3-aminopropyl)-4,5-dihydro-2H-pyrazolo[3,4-f]isoquinoline-3-carboxylic acid trifluoroacetate,
2-[3-({3-[4-(methylsulfonyl)phenyl]propyl}amino)propyl]-4,5-dihydro-2H-pyrazolo[3,4-f]isoquinoline-3-carboxylic acid trifluoroacetate,
2-(3-aminopropyl)-8-(2-methoxyphenyl)-4,5-dihydro-2H-pyrazolo[3,4-f]isoquinoline-3-carboxylic acid trifluoroacetate,
2-[3-({2-[3′-(aminomethyl)-1,1′-biphenyl-4-yl]ethyl}amino)propyl]-4,5-dihydro-2H-pyrazolo[3,4-f]isoquinoline-3-carboxylic acid trifluoroacetate,
2-(2-aminoethyl)-4,5-dihydro-2H-pyrazolo[3,4-f]isoquinoline-3-carboxylic acid dihydrochloride,
2-(3-{[2-(4-nitrophenyl)ethyl]amino}propyl)-4,5-dihydro-2H-pyrazolo[3,4-f]isoquinoline-3-carboxylic acid trifluoroacetate,
2-[3-({2-[2′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
9-(hydroxymethyl)-5,6,9,10-tetrahydropyrazino[1′,2′:1,5]pyrazolo[3,4-f]isoquinolin-7(8H)-one,
2-(3-{[2-(4-methylphenyl)ethyl]amino}propyl)-4,5-dihydro-2H-pyrazolo[3,4-f]isoquinoline-3-carboxylic acid trifluoroacetate,
9-{[(2-thien-2-ylethyl)amino]methyl}-5,6,9,10-tetrahydropyrazino[1′,2′:1,5]pyrazolo[3,4-f]isoquinolin-7(8H)-one,
2-(3-{[2-(4-ethoxyphenyl)ethyl]amino}propyl)-4,5-dihydro-2H-pyrazolo[3,4-f]isoquinoline-3-carboxylic acid trifluoroacetate,
2-(1,3-benzodioxol-5-yl)-5,6,8,9,10,11-hexahydro-7H-[1,4]diazepino[1′,2′:1,5]pyrazolo[3,4-f]isoquinolin-7-one,
2-(3-{[2-(4-methoxyphenyl)ethyl]amino}propyl)-4,5-dihydro-2H-pyrazolo[3,4-f]isoquinoline-3-carboxylic acid trifluoroacetate,
3-[3-(1H-tetraazol-5-yl)-4,5-dihydro-2H-pyrazolo[3,4-f]isoquinolin-2-yl]propan-1-amine hydrochloride,
2-(3-aminopropyl)-8-chloro-4,5-dihydro-2H-pyrazolo[3,4-f]isoquinoline-3-carboxylic acid trifluoroacetate,
2-(3-{[(1R,2S)-2-phenylcyclopropyl]amino}propyl)-4,5-dihydro-2H-pyrazolo[3,4-f]isoquinoline-3-carboxylic acid trifluoroacetate,
2-{3-[(3,3-diphenylpropyl)amino]propyl}-4,5-dihydro-2H-pyrazolo[3,4-f]isoquinoline-3-carboxylic acid trifluoroacetate,
2-(3-{[2-(3-bromo-4-methoxyphenyl)ethyl]amino}propyl)-4,5-dihydro-2H-pyrazolo[3,4-f]isoquinoline-3-carboxylic acid trifluoroacetate,
2-{3-[(4-phenylbutyl)amino]propyl}-4,5-dihydro-2H-pyrazolo[3,4-f]isoquinoline-3-carboxylic acid trifluoroacetate,
2-[3-(2,3-dihydro-1H-inden-2-ylamino)propyl)-4,5-dihydro-2H-pyrazolo[3,4-f]isoquinoline-3-carboxylic acid trifluoroacetate,
2-(2-naphthyl)-5,6,8,9,10,11-hexahydro-7H-[1,4]diazepino[1′,2′:1,5]pyrazolo[3,4-f]isoquinolin-7-one,
2-{3-[(3-phenylpropyl)amino]propyl}-4,5-dihydro-2H-pyrazolo[3,4-f]isoquinoline-3-carboxylic acid trifluoroacetate,
2-(3-{[2-(4-fluorophenyl)ethyl]amino}propyl)-4,5-dihydro-2H-pyrazolo[3,4-f]isoquinoline-3-carboxylic acid trifluoroacetate,
2-{3-[(2-thien-3-ylethyl)amino]propyl}-4,5-dihydro-2H-pyrazolo[3,4-f]isoquinoline-3-carboxylic acid dihydrochloride,
5,6,9,10-tetrahydropyrazino[1′,2′:1,5]pyrazolo[3,4-f]isoquinolin-7(8H)-one,
2-[3-(glycoloylamino)propyl]-8-quinolin-3-yl-4,5-dihydro-2H-pyrazolo[3,4-f]isoquinoline-3-carboxylic acid hydrochloride,
8-quinolin-3-yl-4,5-dihydro-2H-pyrazolo[3,4-f]isoquinoline-3-carboxylic acid,
2-(3-{[2-(4-ethylphenyl)ethyl]amino}propyl)-4,5-dihydro-2H-pyrazolo[3,4-f]isoquinoline-3-carboxylic acid trifluoroacetate,
2-(3-{[2-(2-chlorophenyl)ethyl]amino}propyl)-4,5-dihydro-2H-pyrazolo[3,4-f]isoquinoline-3-carboxylic acid trifluoroacetate,
2-{3-[(2-ethylbutyl)amino]propyl}-4,5-dihydro-2H-pyrazolo[3,4-f]isoquinoline-3-carboxylic acid trifluoroacetate,
8-quinolin-3-yl-4,5-dihydro-2H-pyrazolo[3,4-q]isoquinoline-3-carboxamide,
2-{3-[(2-pyridin-4-ylethyl)amino]propyl}-4,5-dihydro-2H-pyrazolo[3,4-f]isoquinoline-3-carboxylic acid trifluoroacetate,
2-(3-{[2-(4-chlorophenyl)propyl]amino}propyl)-4,5-dihydro-2H-pyrazolo[3,4-f]isoquinoline-3-carboxylic acid trifluoroacetate,
2-(3-{[2-(3-chlorophenyl)ethyl]amino}propyl)-4,5-dihydro-2H-pyrazolo[3,4-f]isoquinoline-3-carboxylic acid trifluoroacetate,
2-[3-(glycoloylamino)propyl]-4,5-dihydro-2H-pyrazolo[3,4-f]isoquinoline-3-carboxylic acid trifluoroacetate,
2-(3-{[2-(3,4-dimethoxyphenyl)ethyl]amino}propyl)-4,5-dihydro-2H-pyrazolo[3,4-f]isoquinoline-3-carboxylic acid trifluoroacetate,
2-(1-benzofuran-2-yl)-5,6,8,9,10,11-hexahydro-7H-[1,4]diazepino[1′,2′:1,5]pyrazolo[3,4-f]isoquinolin-7-one,
2-(3-{[4-(2-aminoethyl)phenyl]amino}propyl)-4,5-dihydro-2H-pyrazolo[3,4-f]isoquinoline-3-carboxylic acid trifluoroacetate,
2-(1-naphthyl)-5,6,9,10-tetrahydropyrazino[1′,2′:1,5]pyrazolo[3,4-f]isoquinolin-7(8H)-one,
8-(3-aminopropyl)-5,6,8,9,10,11-hexahydro-7H-[1,4]diazepino[1′,2′:1,5]pyrazolo[3,4-f]isoquinolin-7-one dihydrochloride,
2-anilino-5,6,9,10-tetrahydropyrazino[1′,2′:1,5]pyrazolo[3,4-f]isoquinolin-7(8H)-one,
2-(3-aminopropyl)-8-[2-(trifluoromethyl)phenyl]-4,5-dihydro-2H-pyrazolo[3,4-f]isoquinoline-3-carboxylic acid trifluoroacetate,
9-(azidomethyl)-5,6,9,10-tetrahydropyrazino[1′,2′:1,5]pyrazolo[3,4-f]isoquinolin-7(8H)-one,
9-({[2-(4-chlorophenyl)ethyl]amino}methyl)-5,6,9,10-tetrahydropyrazino[1′,2′:1,5]pyrazolo[3,4-f]isoquinolin-7(8H)-one,
2-phenyl-5,6,8,9,10,11-hexahydro-7H-[1,4]diazepino[1′,2′:1,5]pyrazolo[3,4-f]isoquinolin-7-one,
2-[3-(pentylamino)propyl]-4,5-dihydro-2H-pyrazolo[3,4-f]isoquinoline-3-carboxylic acid trifluoroacetate,
10-(2-aminoethyl)-5,6,9,10-tetrahydropyrazino[1′,2′:1,5]pyrazolo[3,4-f]isoquinolin-7(8H)-one,
2-[3-(allylamino)propyl]-4,5-dihydro-2H-pyrazolo[3,4-f]isoquinoline-3-carboxylic acid trifluoroacetate,
2-(4-aminobutyl)-4,5-dihydro-2H-pyrazolo[3,4-f]isoquinoline-3-carboxylic acid dihydrochloride,
2-(3-{[2-(4-aminophenyl)ethyl]amino}propyl)-4,5-dihydro-2H-pyrazolo[3,4-f]isoquinoline-3-carboxylic acid trifluoroacetate,
2-[(1E)-3,3-dimethylbut-1-enyl]-5,6,9,10-tetrahydropyrazino[1′,2′:1,5]pyrazolo[3,4-f]isoquinolin-7(8H)-one trifluoroacetate,
10-(nitromethyl)-5,6,9,10-tetrahydropyrazino[1′,2′:1,5)pyrazolo[3,4-f]isoquinolin-7(8H)-one,
3-carboxy-2-[3-(methylammonio)propyl]-4,5-dihydro-2H-pyrazolo[3,4-f]isoquinolin-7-ium dichloride,
2-[3-({[(4-butoxyphenyl)amino)carbonyl}amino)propyl]-4,5-dihydro-2H-pyrazolo[3,4-f]isoquinoline-3-carboxylic acid,
2-{3-[(2-pyridin-3-ylethyl)amino]propyl}-4,5-dihydro-2H-pyrazolo[3,4-f]isoquinoline-3-carboxylic acid trifluoroacetate,
2-{3-[(2-pyridin-2-ylethyl)amino]propyl}-4,5-dihydro-2H-pyrazolo[3,4-f]isoquinoline-3-carboxylic acid trifluoroacetate,
2-{3-[(cyclopropylmethyl)amino]propyl}-4,5-dihydro-2H-pyrazolo[3,4-f]isoquinoline-3-carboxylic acid trifluoroacetate,
2-{3-[(2-thien-2-ylpropyl)amino]propyl}-4,5-dihydro-2H-pyrazolo[3,4-f]isoquinoline-3-carboxylic acid trifluoroacetate,
2-methoxy-5,6,9,10-tetrahydropyrazino[1′,2′:1,5]pyrazolo[3,4-f]isoquinolin-7(8H)-one,
2-[3-(dimethylamino)phenyl]-5,6,9,10-tetrahydropyrazino[1′,2′:1,5]pyrazolo[3,4-f]isoquinolin-7(8H)-one,
2-(3-{[2-(1H-pyrrol-1-yl)ethyl]amino}propyl)-4,5-dihydro-2H-pyrazolo[3,4-f]isoquinoline-3-carboxylic acid trifluoroacetate,
2-[3-(benzyloxy)propyl]-8-quinolin-3-yl-4,5-dihydro-2H-pyrazolo[3,4-f]isoquinoline-3-carboxylic acid,
2-{3-[(4-butoxybenzyl)amino]propyl}-4,5-dihydro-2H-pyrazolo[3,4-f]isoquinoline-3-carboxylic acid trifluoroacetate,
2-(1,3-benzodioxol-5-yl)-5,6,9,10-tetrahydropyrazino[1′,2′:1,5]pyrazolo[3,4-f]isoquinolin-7(8H)-one,
2-[(E)-2-(2-fluorophenyl)ethenyl]-5,6,9,10-tetrahydropyrazino[1′,2′:1,5]pyrazolo[3,4-f]isoquinolin-7(8H)-one,
2-[(1E)-hex-1-enyl]-5,6,9,10-tetrahydropyrazino[1′,2′:1,5]pyrazolo[3,4-f]isoquinolin-7(8H)-one trifluoroacetate,
2-anilino-5,6,8,9,10,11-hexahydro-7H-[1,4]diazepino[1′,2′:1,5]pyrazolo[3,4-f]isoquinolin-7-one,
5,6,8,9,10,11-hexahydro-7H-[1,4]diazepino[1′,2′:1,5]pyrazolo[3,4-f]isoquinolin-7-one,
2-chloro-5,6,9,10-tetrahydropyrazino[1′,2′:1,5]pyrazolo[3,4-f]isoquinolin-7(8H)-one,
2-[(E)-2-(4-methoxyphenyl)ethenyl]-5,6,9,10-tetrahydropyrazino[1′,2′:1,5]pyrazolo[3,4-f]isoquinolin-7(8H)-one,
2-(methylthio)-5,6,9,10-tetrahydropyrazino[1′,2′:1,5]pyrazolo[3,4-f]isoquinolin-7(8H)-one,
2-{3-[(2-furylmethyl)amino]propyl}-4,5-dihydro-2H-pyrazolo[3,4-f]isoquinoline-3-carboxylic acid trifluoroacetate,
2-azepan-1-yl-5,6,9,10-tetrahydropyrazino[1′,2′:1,5]pyrazolo[3,4-f]isoquinolin-7(8H)-one trifluoroacetate,
2-(3,6-dihydropyridin-1 (2H)-yl)-5,6,9,10-tetrahydropyrazino[1′,2′:1,5]pyrazolo[3,4-f]isoquinolin-7(8H)-one trifluoroacetate,
9-methyl-5,6,9,10-tetrahydropyrazino[1′,2′:1,5]pyrazolo[3,4-f]isoquinolin-7(8H)-one,
2-(piperidin-3-ylmethyl)-4,5-dihydro-2H-pyrazolo[3,4-f]isoquinoline-3-carboxylic acid hydrochloride,
9-(chloromethyl)-5,6,9,10-tetrahydropyrazino[1′,2′:1,5]pyrazolo[3,4-f]isoquinolin-7(8H)-one,
2-[(4-methoxybenzyl)amino]-5,6,9,10-tetrahydropyrazino[1′,2′:1,5]pyrazolo[3,4-f]isoquinolin-7(8H)-one trifluoroacetate,
2-(3-{[2-(1H-imidazol-4-yl)ethyl]amino}propyl)-4,5-dihydro-2H-pyrazolo[3,4-f]isoquinoline-3-carboxylic acid trifluoroacetate,
2-(benzylamino)-5,6,9,10-tetrahydropyrazino[1′,2′:1,5]pyrazolo[3,4-f]isoquinolin-7(8H)-one trifluoroacetate,
2-(methylthio)-5,6,8,9,10,11-hexahydro-7H-[1,4]diazepino[1′,2′:1,5]pyrazolo[3,4-f]isoquinolin-7-one,
2-{3-[(2-chlorobenzyl)amino]propyl}-4,5-dihydro-2H-pyrazolo[3,4-f]isoquinoline-3-carboxylic acid trifluoroacetate,
2-[3-(benzylamino)propyl]-4,5-dihydro-2H-pyrazolo[3,4-f]isoquinoline-3-carboxylic acid trifluoroacetate,
2-[3-({[(4-methoxyphenyl)amino]carbonyl}amino)propyl]-4,5-dihydro-2H-pyrazolo[3,4-f]isoquinoline-3-carboxylic acid,
2-{3-[(2-phenylethyl)amino]propyl}-4,5-dihydro-2H-pyrazolo[3,4-f]isoquinoline-3-carboxylic acid trifluoroacetate,
2-{3-[(thien-2-ylmethyl)amino]propyl}-4,5-dihydro-2H-pyrazolo[3,4-f]isoquinoline-3-carboxylic acid trifluoroacetate,
2-benzyl-5,6,9,10-tetrahydropyrazino[1′,2′:1,5]pyrazolo[3,4-f]isoquinolin-7(8H)-one,
5,6,8,9,10,11-hexahydro-7H-[1,4]diazepino[1′,2′:1,5]pyrazolo[3,4-f]isoquinolin-7-one,
2-{3-[(4-chlorobenzyl)amino]propyl}-4,5-dihydro-2H-pyrazolo[3,4-f]isoquinoline-3-carboxylic acid trifluoroacetate,
2-{3-[(2-phenylpropyl)amino]propyl}-4,5-dihydro-2H-pyrazolo[3,4-f]isoquinoline-3-carboxylic acid trifluoroacetate,
7-oxo-5,6,7,8,9,10-hexahydropyrazino[1′,2′:1,5]pyrazolo[3,4-f]isoquinoline-9-carboxamide trifluoroacetate,
2-(3-hydroxypropyl)-4,5-dihydro-2H-pyrazolo[3,4-f]isoquinoline-3-carboxylic acid,
2-(1,3-dihydro-2H-isoindol-2-yl)-5,6,9,10-tetrahydropyrazino[1′,2′:1,5]pyrazolo[3,4-f]isoquinolin-7(8H)-one trifluoroacetate,
2-{3-[(4-aminophenyl)amino]propyl}-4,5-dihydro-2H-pyrazolo[3,4-f]isoquinoline-3-carboxylic acid trifluoroacetate,
2-(4-hydroxypiperidin-1-yl)-5,6,9,10-tetrahydropyrazino[1′,2′:1,5]pyrazolo[3,4-f]isoquinolin-7(8H)-one trifluoroacetate,
2-{3-[(1H-imidazol-5-ylmethyl)amino]propyl}-4,5-dihydro-2H-pyrazolo[3,4-f]isoquinoline-3-carboxylic acid trifluoroacetate,
2-{3-[(3-chlorobenzyl)amino]propyl}-4,5-dihydro-2H-pyrazolo[3,4-f]isoquinoline-3-carboxylic acid trifluoroacetate,
2-{3-[(pyridin-3-ylcarbonyl)amino]propyl}-4,5-dihydro-2H-pyrazolo[3,4-f]isoquinoline-3-carboxylic acid trifluoroacetate,
4,5-dihydro-2H-pyrazolo[3,4-f]isoquinoline-3-carboxamide,
2-[3-(cyclopentylamino)propyl]-4,5-dihydro-2H-pyrazolo[3,4-f]isoquinoline-3-carboxylic acid dihydrochloride,
2-(3-{[2-(1H-indol-3-yl)ethyl]amino}propyl)-4,5-dihydro-2H-pyrazolo[3,4-f]isoquinoline-3-carboxylic acid,
10-(3-aminopropyl)-5,6,9,10-tetrahydropyrazino[1′,2′:1,5]pyrazolo[3,4-f]isoquinolin-7(8H)-one,
9-(1,2-dihydroxyethyl)-5,6,9,10-tetrahydropyrazino[1′,2′:1,5]pyrazolo[3,4-f]isoquinolin-7(8H)-one,
2-morpholin-4-yl-5,6,9,10-tetrahydropyrazino[1′,2′:1,5]pyrazolo[3,4-f]isoquinolin-7(8H)-one trifluoroacetate,
ethyl 2-(2-aminoethyl)-4,5-dihydro-2H-pyrazolo[3,4-f]isoquinoline-3-carboxylate dihydrochloride,
2-allyl-4,5-dihydro-2-H-pyrazolo[3,4-f]isoquinoline-3-carboxylic acid,
2-quinolin-8-yl-5,6,9,10-tetrahydropyrazino[1′,2′:1,5]pyrazolo[3,4-f]isoquinolin-7(8H)-one,
lithium 2-[3-(2,3-dihydro-1H-inden-2-ylamino)-2-hydroxypropyl]-4,5-dihydro-²H-pyrazolo[3,4-f]isoquinoline-3-carboxylate,
2-(2-hydroxyethyl)-4,5-dihydro-2H-pyrazolo[3,4-f]isoquinoline-3-carboxylic acid,
2-{3-[(2-pyrrolidin-1-ylethyl)amino]propyl}-4,5-dihydro-2H-pyrazolo[3,4-f]isoquinoline-3-carboxylic acid trifluoroacetate,
2-(3,4-dihydroisoquinolin-2(1H)-yl)-5,6,9,10-tetrahydropyrazino[1′,2′:1,5]pyrazolo[3,4-f]isoquinolin-7(8H)-one trifluoroacetate,
2-(3-{[(benzylamino)carbonyl]amino}propyl)-4,5-dihydro-2H-pyrazolo[3,4-f]isoquinoline-3-carboxylic acid,
2-[3-(dimethylamino)propyl]-4,5-dihydro-2H-pyrazolo[3,4-f]isoquinoline-3-carboxylic acid trifluoroacetate,
10-(3-{[2-(4-chlorophenyl)ethyl]amino}propyl)-5,6,9,10-tetrahydropyrazino[1′,2′:1,5]pyrazolo[3,4-f]isoquinolin-7(8H)-one,
2-(3-aminopropyl)-6-chloro-4,5-dihydro-2H-pyrazolo[3,4-f]isoquinoline-3-carboxylic acid trifluoroacetate,
methyl 7-oxo-5,6,7,8,9,10-hexahydropyrazino[1′,2′:1,5]pyrazolo[3,4-f]isoquinoline-9-carboxylate,
2-[2-(glycoloylamino)ethyl]-4,5-dihydro-2H-pyrazolo[3,4-f]isoquinoline-3-carboxylic acid hydrochloride,
2-[2-(isopropylamino)ethyl]-4,5-dihydro-2H-pyrazolo[3,4-f]isoquinoline-3-carboxylic acid hydrochloride,
2-(3-cyanopropyl)-4,5-dihydro-2H-pyrazolo[3,4-f]isoquinoline-3-carboxylic acid,
2-[3-({[(3-cyanophenyl)amino]carbonyl}amino)propyl]-4,5-dihydro-2H-pyrazolo[3,4-f]isoquinoline-3-carboxylic acid,
9-(aminomethyl)-5,6,9,10-tetrahydro-7H-[1,4]oxazino[4′,3′:1,5]pyrazolo[3,4-f]isoquinolin-7-one trifluoroacetate,
2-(3-aminopropyl)-7-hydroxy-8-(3-nitrophenyl)-4,5-dihydro-2H-benzo[g]indazole-3-carboxylic acid trifluoroacetate,
2-(2-aminoethyl)-7-hydroxy-8-(3-nitrophenyl)-4,5-dihydro-2H-benzo[g]indazole-3-carboxylic acid trifluoroacetate,
3-hydroxy-2-(3-nitrophenyl)-5,6,8,9,10,11-hexahydro-7H-benzo[g][1,4]diazepino[1,2-b]indazol-7-one trifluoroacetate,
2-(3-aminopropyl)-7-hydroxy-4,5-dihydro-2H-benzo[g]indazole-3-carboxylic acid dihydrochloride,
2-(2-aminoethyl)-8-bromo-7-hydroxy-4,5-dihydro-2H-benzo[g]indazole-3-carboxylic acid trifluoroacetate,
2-(3-aminopropyl)-8-[2-(4-chlorophenyl)ethyl]-7-hydroxy-4,5-dihydro-2H-benzo[g]indazole-3-carboxylic acid trifluoroacetate,
3-hydroxy-2-(3-nitrophenyl)-5,6,9,10-tetrahydrobenzo[g]pyrazino[1,2-b]indazol-7(8H)-one hydrobromide,
2-(3-aminopropyl)-8-bromo-7-hydroxy-4,5-dihydro-2H-benzo[g]indazole-3-carboxylic acid hydrobromide,
2-(3-aminopropyl)-7-hydroxy-8-(4-nitrophenyl)-4,5-dihydro-2H-benzo[g]indazole-3-carboxylic acid trifluoroacetate,
2-(3-aminopropyl)-8-(3-cyanophenyl)-7-hydroxy-4,5-dihydro-2H-benzo[g]indazole-3-carboxylic acid trifluoroacetate,
2-(3-aminopropyl)-7-hydroxy-8-[3-(trifluoromethyl)phenyl]-4,5-dihydro-2H-benzo[g]indazole-3-carboxylic acid trifluoroacetate,
2-(3-aminopropyl)-7-hydroxy-8-(3,3,3-trifluoropropyl)-4,5-dihydro-2H-benzo[g]indazole-3-carboxylic acid trifluoroacetate,
2-(3-aminopropyl)-8-(3-chlorophenyl)-7-hydroxy-4,5-dihydro-2H-benzo[g]indazole-3-carboxylic acid trifluoroacetate,
2-(3-aminopropyl)-7-hydroxy-5-methyl-4,5-dihydro-2H-benzo[g]indazole-3-carboxylic acid trifluoroacetate,
2-(3-aminopropyl)-8-(4-chlorophenyl)-7-hydroxy-4,5-dihydro-2H-benzo[g]indazole-3-carboxylic acid trifluoroacetate,
2-(3-aminopropyl)-7-hydroxy-8-[3-(methylsulfonyl)phenyl]-4,5-dihydro-2H-benzo[g]indazole-3-carboxylic acid trifluoroacetate,
2-(3-aminopropyl)-8-(3,4-difluorophenyl)-7-hydroxy-4,5-dihydro-2H-benzo[g]indazole-3-carboxylic acid trifluoroacetate,
3-hydroxy-5,6,8,9,10,11-hexahydro-7H-benzo[g][1,4]diazepino[1,2-b]indazol-7-one,
8-(3-aminophenyl)-2-(3-aminopropyl)-7-hydroxy-4,5-dihydro-2H-benzo[g]indazole-3-carboxylic acid trifluoroacetate,
6-bromo-7-hydroxy-4,5-dihydro-2H-benzo[g]indazole-3-carboxamide,
2-(3-aminopropyl)-7-hydroxy-8-[4-(methylamino)phenyl]-4,5-dihydro-2H-benzo[g]indazole-3-carboxylic acid trifluoroacetate,
2-(3-aminopropyl)-8-(4-tert-butylphenyl)-7-hydroxy-4,5-dihydro-2H-benzo[g]indazole-3-carboxylic acid trifluoroacetate,
2-(3-aminopropyl)-7-hydroxy-8-pyridin-4-yl-4,5-dihydro-2H-benzo[g]indazole-3-carboxylic acid trifluoroacetate, and
7-hydroxy-4,5-dihydro-2H-benzo[g]indazole-3-carboxylic acid.

20. The cyclic pyrazole MK-2 inhibitor according to claim 1, wherein the MK-2 inhibitor has an MK-2 IC₅₀ of less than 10 and is selected from the group consisting of:
2-(3-aminopropyl)-8-(methylthio)-2,4,5,6-tetrahydropyrazolo[3,4-e]indazole-3-carboxylic acid dihydrochloride,
2-(3-aminopropyl)-2,4,5,6-tetrahydropyrazolo[3,4-e]indazole-3-carboxylic acid hydrochloride,
2-(3-{[2-(4-bromophenyl)ethyl]amino}propyl)-2,4,5,6-tetrahydropyrazolo[3,4-e]indazole-3-carboxylic acid hydrochloride,
2-(2-aminoethyl)-2,4,5,6-tetrahydropyrazolo[3,4-e]indazole-3-carboxylic acid hydrochloride,
8-(allylthio)-2-(3-aminopropyl)-2,4,5,6-tetrahydropyrazolo[3,4-e]indazole-3-carboxylic acid dihydrochloride,
2-(3-aminopropyl)-8-(benzylthio)-2,4,5,6-tetrahydropyrazolo[3,4-e]indazole-3-carboxylic acid dihydrochloride,
2-{3-[(2-thien-2-ylethyl)amino]propyl}-2,4,5,6-tetrahydropyrazolo[3,4-e]indazole-3-carboxylic acid,
2-{3-[(2-thien-3-ylethyl)amino]propyl}-2,4,5,6-tetrahydropyrazolo[3,4-e]indazole-3-carboxylic acid hydrochloride,
ethyl 2-(3-{[2-(4-bromophenyl)ethyl]amino}propyl)-2,4,5,6-tetrahydropyrazolo[3,4-e]indazole-3-carboxylate,
2-(3-aminopropyl)-8-quinolin-3-yl-4,5-dihydro-2H-pyrazolo[4,3-h]quinazoline-3-carboxylic acid dihydrochloride,
2-(3-aminopropyl)-8-(1,3-benzodioxol-5-yl)-4,5-dihydro-2H-pyrazolo[4,3-h]quinazoline-3-carboxylic acid hydrochloride,
2-(3-aminopropyl)-8-phenyl-4,5-dihydro-2H-pyrazolo[4,3-h]quinazoline-3-carboxylic acid hydrochloride,
2-quinolin-3-yl-5,6,8,9,10,11-hexahydro-7H-[1,4]diazepino[1′,2′:1,5]pyrazolo[4,3-h]quinazolin-7-one,
2-pyridin-3-yl-5,6,8,9,10,11-hexahydro-7H-(1,4]diazepino[1′,2′:1,5]pyrazolo[4,3-h]quinazolin-7-one,
8-quinolin-3-yl-2-[3-(tritylamino)propyl]-4,5-dihydro-2H-pyrazolo[4,3-h]quinazoline-3-carboxylic acid hydrochloride,
2-(1,3-benzodioxol-5-yl)-5,6,8,9,10,11-hexahydro-7H-[1,4]diazepino[1′,2′:1,5]pyrazolo[4,3-h]quinazolin-7-one,
2-(4-methoxyphenyl)-5,6,8,9,10,11-hexahydro-7H-[1,4]diazepino[1′,2′:1,5]pyrazolo[4,3-h]quinazolin-7-one,
2-pyridin-4-yl-5,6,8,9,10,11-hexahydro-7H-[1,4]diazepino[1′,2′:1,5]pyrazolo[4,3-h]quinazolin-7-one hydrochloride,
2-(3-aminopropyl)-8-quinolin-3-yl-4,5-dihydro-2H-pyrazolo[3,4-f]isoquinoline-3-carboxylic acid trifluoroacetate,
2-(3-aminopropyl)-8-(3-nitrophenyl)-4,5-dihydro-2H-pyrazolo[3,4-f]isoquinoline-3-carboxylic acid trifluoroacetate,
2-(3-aminopropyl)-8-(4-hydroxyphenyl)-4,5-dihydro-2H-pyrazolo[3,4-f]isoquinoline-3-carboxylic acid hydrobromide,
2-(3-aminopropyl)-8-(3-hydroxyphenyl)-4,5-dihydro-2H-pyrazolo[3,4-f]isoquinoline-3-carboxylic acid hydrobromide,
2-(3-aminopropyl)-8-(2-naphthyl)-4,5-dihydro-2H-pyrazolo[3,4-f]isoquinoline-3-carboxylic acid trifluoroacetate,
2-(3-aminopropyl)-8-(3,5-difluorophenyl)-4,5-dihydro-2H-pyrazolo[3,4-f]isoquinoline-3-carboxylic acid trifluoroacetate,
2-(3-aminopropyl)-8-(1,3-benzodioxol-5-yl)-4,5-dihydro-2H-pyrazolo[3,4-q]isoquinoline-3-carboxylic acid trifluoroacetate,
2-(3-aminopropyl)-8-(3-cyanophenyl)-4,5-dihydro-2H-pyrazolo[3,4-q]isoquinoline-3-carboxylic acid trifluoroacetate,
9-(hydroxymethyl)-2-quinolin-3-yl-5,6,9,10-tetrahydropyrazino[1′,2′:1,5]pyrazolo[3,4-f]isoquinolin-7(8H)-one trifluoroacetate,
2-(3-aminopropyl)-8-(4-methoxyphenyl)-4,5-dihydro-2H-pyrazolo[3,4-q]isoquinoline-3-carboxylic acid trifluoroacetate,
2-(3-aminopropyl)-8-[3-(methylsulfonyl)phenyl]-4,5-dihydro-2H-pyrazolo[3,4-q]isoquinoline-3-carboxylic acid trifluoroacetate,
2-(3-aminopropyl)-8-[3-(trifluoromethyl)phenyl]-4,5-dihydro-2H-pyrazolo[3,4-q]isoquinoline-3-carboxylic acid trifluoroacetate,
2-(1H-imidazol-1-yl)-5,6,9,10-tetrahydropyrazino[1′,2′:1,5]pyrazolo[3,4-q]isoquinolin-7(8H)-one trifluoroacetate,
2-(3-aminopropyl)-8-(3-methoxyphenyl)-4,5-dihydro-2H-pyrazolo[3,4-f]isoquinoline-3-carboxylic acid trifluoroacetate,
2-(3-aminopropyl)-8-[4-(trifluoromethyl)phenyl]-4,5-dihydro-2H-pyrazolo[3,4-f]isoquinoline-3-carboxylic acid trifluoroacetate,
2-(3-aminopropyl)-8-anilino-4,5-dihydro-2H-pyrazolo[3,4-f]isoquinoline-3-carboxylic acid,
2-(3-aminopropyl)-8-(3,4-difluorophenyl)-4,5-dihydro-2H-pyrazolo[3,4-f]isoquinoline-3-carboxylic acid trifluoroacetate,
2-(3-{[2-(4′-carboxy-1,1′-biphenyl-4-yl)ethyl]amino}propyl)-4,5-dihydro-2H-pyrazolo[3,4-f)isoquinoline-3-carboxylic acid trifluoroacetate,
2-(4-hydroxyphenyl)-5,6,9,10-tetrahydropyrazino[1′,2′:1,5]pyrazolo[3,4-f]isoquinolin-7(8H)-one trifluoroacetate,
2-propyl-8-quinolin-3-yl-4,5-dihydro-2H-pyrazolo[3,4-f]isoquinoline-3-carboxylic acid,
2-[3-({2-[3′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
2-(3-aminopropyl)-8-(4-tert-butylphenyl)-4,5-dihydro-2H-pyrazolo[3,4-f]isoquinoline-3-carboxylic acid trifluoroacetate,
2-[3-({2-[4-(3-furyl)phenyl]ethyl}amino)propyl]-4,5-dihydro-2H-pyrazolo[3,4-f]isoquinoline-3-carboxylic acid trifluoroacetate,
2-(3-{[2-(3′-chloro-1,1′-biphenyl-4-yl)ethyl]amino}propyl)-4,5-dihydro-2H-pyrazolo[3,4-f]isoquinoline-3-carboxylic acid trifluoroacetate,
2-(4-methoxyphenyl)-5,6,8,9,10,11-hexahydro-7H-[1,4]diazepino[1′,2′:1,5]pyrazolo[3,4-f]isoquinolin-7-one,
2-[3-({2-[4′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
2-(3-hydroxyphenyl)-5,6,9,10-tetrahydropyrazino[1′,2′-:1,5]pyrazolo[3,4-f]isoquinolin-7(8H)-one,
2-(3-aminopropyl)-N-hydroxy-8-quinolin-3-yl-4,5-dihydro-2H-pyrazolo[3,4-f]isoquinoline-3-carboxamide hydrochloride,
2-[(E)-2-(4-hydroxyphenyl)ethenyl]-5,6,9,10-tetrahydropyrazino[1′,2′:1,5]pyrazolo[3,4-f]isoquinolin-7(8H)-one trifluoroacetate,
2-quinolin-3-yl-5,6,8,9,10,11-hexahydro-7H-[1,4]diazepino[1′,2′:1,5]pyrazolo[3,4-f]isoquinolin-7-one,
2-(4-hydroxyphenyl)-5,6,8,9,10,11-hexahydro-7H-[1,4]diazepino[1′,2′:1,5]pyrazolo[3,4-f]isoquinolin-7-one,
2-(3-{[2-(2′-chloro-1,1′-biphenyl-4-yl)ethyl]amino}propyl)-4,5-dihydro-2H-pyrazolo[3,4-f]isoquinoline-3-carboxylic acid trifluoroacetate,
2-(3-{[2-(4′-tert-butyl-1,1′-biphenyl-4-yl)ethyl]amino}propyl)-4,5-dihydro-2H-pyrazolo[3,4-f]isoquinoline-3-carboxylic acid trifluoroacetate,
2-(3-{[2-(3,4-dichlorophenyl)ethyl]amino}propyl)-4,5-dihydro-2H-pyrazolo[3,4-f]isoquinoline-3-carboxylic acid trifluoroacetate,
2-(3-{[3-(3-chlorophenyl)propyl]amino}propyl)-4,5-dihydro-2H-pyrazolo[3,4-f]isoquinoline-3-carboxylic acid trifluoroacetate,
2-[(E)-2-phenylethenyl]-5,6,9,10-tetrahydropyrazino[1′,2′:1,5]pyrazolo[3,4-f]isoquinolin-7(8H)-one trifluoroacetate,
2-(3-{[2-(4-pyridin-4-ylphenyl)ethyl]amino}propyl)-4,5-dihydro-2H-pyrazolo[3,4-f]isoquinoline-3-carboxylic acid trifluoroacetate,
2-(3-{[3-(4-bromophenyl)propyl]amino}propyl)-4,5-dihydro-2H-pyrazolo[3,4-f]isoquinoline-3-carboxylic acid trifluoroacetate,
2-(3-{[3-(4-tert-butylphenyl)propyl]amino}propyl)-4,5-dihydro-2H-pyrazolo[3,4-f]isoquinoline-3-carboxylic acid trifluoroacetate,
2-(3-{[2-(3′-isopropyl-1,1′-biphenyl-4-yl)ethyl]amino}propyl)-4,5-dihydro-2H-pyrazolo[3,4-f]isoquinoline-3-carboxylic acid trifluoroacetate,
2-{3-[(2-thien-2-ylethyl)amino]propyl}-4,5-dihydro-2H-pyrazolo[3,4-f]isoquinoline-3-carboxylic acid trifluoroacetate,
2-[4-(dimethylamino)phenyl]-5,6,9,10-tetrahydropyrazino[1′,2′:1,5]pyrazolo[3,4-f]isoquinolin-7(8H)-one,
2-(3-{[2-(1,1′-biphenyl-4-yl)ethyl]amino}propyl)-4,5-dihydro-2H-pyrazolo[34-f]isoquinoline-3-carboxylic acid trifluoroacetate,
2-(3-methoxyphenyl)-5,6,9,10-tetrahydropyrazino[1′,2′:1,5]pyrazolo[3,4-f]isoquinolin-7(8H)-one,
2-(3-{[2-(4-bromophenyl)ethyl]amino}propyl)-4,5-dihydro-2H-pyrazolo[3,4-f]isoquinoline-3-carboxylic acid trifluoroacetate,
2-(3-{[2-(2,4-dichlorophenyl)ethyl]amino}propyl)-4,5-dihydro-2H-pyrazolo[3,4-f]isoquinoline-3-carboxylic acid trifluoroacetate,
2-{3-[(benzylsulfonyl)amino]propyl}-8-quinolin-3-yl-4,5-dihydro-2H-pyrazolo[3,4-f]isoquinoline-3-carboxylic acid,
9-(aminomethyl)-5,6,9,10-tetrahydropyrazino[1′,2′:1,5]pyrazolo[3,4-f]isoquinolin-7(8H)-one trifluoroacetate,
2-(3-nitrophenyl)-5,6,8,9,10,11-hexahydro-7H-[1,4]diazepino[1′,2′:1,5]pyrazolo[3,4-f]isoquinolin-7-one,
2-(3-aminopropyl)-8-quinolin-3-yl-4,5-dihydro-2H-pyrazolo[3,4-f]isoquinoline-3-carboxamide hydrochloride,
2-(3-{[3-(4-chlorophenyl)propyl]amino}propyl)-4,5-dihydro-2H-pyrazolo[3,4-f]isoquinoline-3-carboxylic acid trifluoroacetate,
2-[3-(dimethylamino)phenyl]-5,6,8,9,10,11-hexahydro-7H-[1,4]diazepino[1′,2′:1,5]pyrazolo[3,4-f]isoquinolin-7-one,
2-(3-{[(4-chlorobenzyl)sulfonyl]amino}propyl)-8-quinolin-3-yl-4,5-dihydro-2H-pyrazolo[3,4-f]isoquinoline-3-carboxylic acid,
2-(3-{[2-(4-pyridin-3-ylphenyl)ethyl]amino}propyl)-4,5-dihydro-2H-pyrazolo[3,4-f]isoquinoline-3-carboxylic acid trifluoroacetate,
2-(3-{[2-(4-chlorophenyl)ethyl]amino}propyl)-4,5-dihydro-2H-pyrazolo[3,4-f]isoquinoline-3-carboxylic acid trifluoroacetate,
2-(3-{[2-(5-chlorothien-2-yl)ethyl]amino}propyl)-4,5-dihydro-2H-pyrazolo[3,4-f]isoquinoline-3-carboxylic acid trifluoroacetate,
2-(3-{[3-(4-cyanophenyl)propyl]amino}propyl)-4,5-dihydro-2H-pyrazolo[3,4-f]isoquinoline-3-carboxylic acid trifluoroacetate,
2-(3-{[3-(5-methyl-2-furyl)butyl]amino}propyl)-4,5-dihydro-2H-pyrazolo[3,4-f]isoquinoline-3-carboxylic acid trifluoroacetate,
2-[3-({2-[4-(1-benzothien-3-yl)phenyl]ethyl}amino)propyl]-4,5-dihydro-2H-pyrazolo[3,4-f]isoquinoline-3-carboxylic acid trifluoroacetate,
2-(3-ammoniopropyl)-3-carboxy-4,5-dihydro-2H-pyrazolo[3,4-f]isoquinolin-7-ium dichloride,
2-(4-methoxyphenyl)-5,6,9,10-tetrahydropyrazino[1′,2′:1,5]pyrazolo[3,4-f]isoquinolin-7(8H)-one,
2-(3-{[3-(4-acetylphenyl)propyl]amino}propyl)-4,5-dihydro-2H-pyrazolo[3,4-f]isoquinoline-3-carboxylic acid trifluoroacetate,
2-(3-aminopropyl)-4,5-dihydro-2H-pyrazolo[3,4-f]isoquinoline-3-carboxylic acid trifluoroacetate,
2-[3-({3-[4-(methylsulfonyl)phenyl]propyl}amino)propyl]-4,5-dihydro-2H-pyrazolo[3,4-f]isoquinoline-3-carboxylic acid trifluoroacetate,
2-(3-aminopropyl)-8-(2-methoxyphenyl)-4,5-dihydro-2H-pyrazolo[3,4-f]isoquinoline-3-carboxylic acid trifluoroacetate,
2-[3-({2-[3′-(aminomethyl)-1,1′-biphenyl-4-yl]ethyl}amino)propyl]-4,5-dihydro-2H-pyrazolo[3,4-f]isoquinoline-3-carboxylic acid trifluoroacetate,
2-(2-aminoethyl)-4,5-dihydro-2H-pyrazolo[3,4-f]isoquinoline-3-carboxylic acid dihydrochloride,
2-(3-{[2-(4-nitrophenyl)ethyl]amino}propyl)-4,5-dihydro-2H-pyrazolo[3,4-f]isoquinoline-3-carboxylic acid trifluoroacetate,
2-[3-({2-[2′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
9-(hydroxymethyl)-5,6,9,10-tetrahydropyrazino[1′,2′:1,5]pyrazolo[3,4-f]isoquinolin-7(8H)-one,
2-(3-{[2-(4-methylphenyl)ethyl]amino}propyl)-4,5-dihydro-2H-pyrazolo[3,4-f]isoquinoline-3-carboxylic acid trifluoroacetate,
9-{[(2-thien-2-ylethyl)amino]methyl}-5,6,9,10-tetrahydropyrazino[1′,2′:1,5]pyrazolo[3,4-f]isoquinolin-7(8H)-one,
2-(3-{[2-(4-ethoxyphenyl)ethyl]amino}propyl)-4,5-dihydro-2H-pyrazolo[3,4-f]isoquinoline-3-carboxylic acid trifluoroacetate,
2-(1,3-benzodioxol-5-yl)-5,6,8,9,10,11-hexahydro-7H-[1,4]diazepino[1′,2′:1,5]pyrazolo[3,4-f]isoquinolin-7-one,
2-(3-{[2-(4-methoxyphenyl)ethyl]amino}propyl)-4,5-dihydro-2H-pyrazolo[3,4-f]isoquinoline-3-carboxylic acid trifluoroacetate,
3-[3-(1H-tetraazol-5-yl)-4,5-dihydro-2H-pyrazolo[3,4-f]isoquinolin-2-yl]propan-1-amine hydrochloride,
2-(3-aminopropyl)-8-chloro-4,5-dihydro-2H-pyrazolo[3,4-q]isoquinoline-3-carboxylic acid trifluoroacetate,
2-(3-{[(1R,2S)-2-phenylcyclopropyl]amino}propyl)-4,5-dihydro-2H-pyrazolo[3,4-f]isoquinoline-3-carboxylic acid trifluoroacetate,
2-{3-[(3,3-diphenylpropyl)amino]propyl}-4,5-dihydro-2H-pyrazolo[3,4-f]isoquinoline-3-carboxylic acid trifluoroacetate,
2-(3-{[2-(3-bromo-4-methoxyphenyl)ethyl]amino}propyl)-4,5-dihydro-2H-pyrazolo[3,4-f]isoquinoline-3-carboxylic acid trifluoroacetate,
2-{3-[(4-phenylbutyl)amino]propyl}-4,5-dihydro-2H-pyrazolo[3,4-f]isoquinoline-3-carboxylic acid trifluoroacetate,
2-[3-(2,3-dihydro-1H-inden-2-ylamino)propyl]-4,5-dihydro-2H-pyrazolo[3,4-f]isoquinoline-3-carboxylic acid trifluoroacetate,
2-(2-naphthyl)-5,6,8,9,10,11-hexahydro-7H-[1,4]diazepino[1′,2′:1,5]pyrazolo[3,4-f]isoquinolin-7-one,
2-{3-[(3-phenylpropyl)amino]propyl}-4,5-dihydro-2H-pyrazolo[3,4-f]isoquinoline-3-carboxylic acid trifluoroacetate,
2-(3-{[2-(4-fluorophenyl)ethyl]amino}propyl)-4,5-dihydro-2H-pyrazolo[3,4-f]isoquinoline-3-carboxylic acid trifluoroacetate,
2-{3-[(2-thien-3-ylethyl)amino]propyl}-4,5-dihydro-2H-pyrazolo[3,4-f]isoquinoline-3-carboxylic acid dihydrochloride,
5,6,9,10-tetrahydropyrazino[1′,2′:1,5]pyrazolo[3,4-f]isoquinolin-7(8H)-one,
2-[3-(glycoloylamino)propyl]-8-quinolin-3-yl-4,5-dihydro-2H-pyrazolo[3,4-f]isoquinoline-3-carboxylic acid hydrochloride,
8-quinolin-3-yl-4,5-dihydro-2H-pyrazolo[3,4-f]isoquinoline-3-carboxylic acid,
2-(3-{[2-(4-ethylphenyl)ethyl]amino}propyl)-4,5-dihydro-2H-pyrazolo[3,4-f]isoquinoline-3-carboxylic acid trifluoroacetate,
2-(3-{[2-(2-chlorophenyl)ethyl]amino}propyl)-4,5-dihydro-2H-pyrazolo[3,4-f]isoquinoline-3-carboxylic acid trifluoroacetate,
2-{3-[(2-ethylbutyl)amino]propyl}-4,5-dihydro-2H-pyrazolo[3,4-f]isoquinoline-3-carboxylic acid trifluoroacetate,
8-quinolin-3-yl-4,5-dihydro-2H-pyrazolo[3,4-f]isoquinoline-3-carboxamide,
2-{3-[(2-pyridin-4-ylethyl)amino]propyl}-4,5-dihydro-2H-pyrazolo[3,4-f]isoquinoline-3-carboxylic acid trifluoroacetate,
2-(3-{[2-(4-chlorophenyl)propyl]amino}propyl)-4,5-dihydro-2H-pyrazolo[3,4-f]isoquinoline-3-carboxylic acid trifluoroacetate,
2-(3-{[2-(3-chlorophenyl)ethyl]amino}propyl)-4,5-dihydro-2H-pyrazolo[3,4-f]isoquinoline-3-carboxylic acid trifluoroacetate,
2-[3-(glycoloylamino)propyl]-4,5-dihydro-2H-pyrazolo[3,4-f]isoquinoline-3-carboxylic acid trifluoroacetate,
2-(3-{[2-(3,4-dimethoxyphenyl)ethyl]amino}propyl)-4,5-dihydro-2H-pyrazolo[3,4-f]isoquinoline-3-carboxylic acid trifluoroacetate,
2-(1-benzofuran-2-yl)-5,6,8,9,10,1,1-hexahydro-7H-[1,4]diazepino[1′,2′:1,5]pyrazolo[3,4-f]isoquinolin-7-one,
2-(3-{[4-(2-aminoethyl)phenyl]amino}propyl)-4,5-dihydro-2H-pyrazolo[3,4-f]isoquinoline-3-carboxylic acid trifluoroacetate,
2-(1-naphthyl)-5,6,9,10-tetrahydropyrazino[1′,2′:1,5]pyrazolo[3,4-f]isoquinolin-7(8H)-one,
8-(3-aminopropyl)-5,6,8,9,10,11-hexahydro-7H-[1,4]diazepino[1′,2′:1,5]pyrazolo[3,4-f]isoquinolin-7-one dihydrochloride,
2-anilino-5,6,9,10-tetrahydropyrazino[1′,2′:1,5]pyrazolo[3,4-f]isoquinolin-7(8H)-one,
2-(3-aminopropyl)-8-[2-(trifluoromethyl)phenyl]-4,5-dihydro-2H-pyrazolo[3,4-f]isoquinoline-3-carboxylic acid trifluoroacetate,
9-(azidomethyl)-5,6,9,10-tetrahydropyrazino[1′,2′:1,5]pyrazolo[3,4-f]isoquinolin-7(8H)-one,
9-({[2-(4-chlorophenyl)ethyl]amino}methyl)-5,6,9,10-tetrahydropyrazino[1′,2′:1,5]pyrazolo[3,4-f]isoquinolin-7(8H)-one,
2-phenyl-5,6,8,9,10,11-hexahydro-7H-[1,4]diazepino[1′,2′:1,5]pyrazolo[3,4-f]isoquinolin-7-one,
2-[3-(pentylamino)propyl]-4,5-dihydro-2H-pyrazolo[3,4-f]isoquinoline-3-carboxylic acid trifluoroacetate,
10-(2-aminoethyl)-5,6,9,10-tetrahydropyrazino[1′,2′:1,5]pyrazolo[3,4-f]isoquinolin-7(8H)-one,
2-[3-(allylamino)propyl]-4,5-dihydro-2H-pyrazolo[3,4-f]isoquinoline-3-carboxylic acid trifluoroacetate,
2-(4-aminobutyl)-4,5-dihydro-2H-pyrazolo[3,4-f]isoquinoline-3-carboxylic acid dihydrochloride,
2-(3-{[2-(4-aminophenyl)ethyl]amino}propyl)-4,5-dihydro-2H-pyrazolo[3,4-f]isoquinoline-3-carboxylic acid trifluoroacetate,
2-[(1E)-3,3-dimethylbut-1-enyl]-5,6,9,10-tetrahydropyrazino[1′,2′:1,5]pyrazolo[3,4-f]isoquinolin-7(8H)-one-trifluoroacetate,
10-(nitromethyl)-5,6,9,10-tetrahydropyrazino[1′,2′:1,5]pyrazolo[3,4-f]isoquinolin-7(8H)-one,
3-carboxy-2-[3-(methylammonio)propyl]-4,5-dihydro-2H-pyrazolo[3,4-f]isoquinolin-7-ium dichloride,
2-[3-({[(4-butoxyphenyl)amino]carbonyl}amino)propyl]-4,5-dihydro-2H-pyrazolo[3,4-f]isoquinoline-3-carboxylic acid,
2-{3-[(2-pyridin-3-ylethyl)amino]propyl}-4,5-dihydro-2H-pyrazolo[3,4-f]isoquinoline-3-carboxylic acid trifluoroacetate,
2-{3-[(2-pyridin-2-ylethyl)amino]propyl}-4,5-dihydro-2H-pyrazolo[3,4-f]isoquinoline-3-carboxylic acid trifluoroacetate,
2-{3-[(cyclopropylmethyl)amino]propyl}-4,5-dihydro-2H-pyrazolo[3,4-f]isoquinoline-3-carboxylic acid trifluoroacetate,
2-{3-[(2-thien-2-ylpropyl)amino]propyl}-4,5-dihydro-2H-pyrazolo[3,4-f]isoquinoline-3-carboxylic acid trifluoroacetate,
2-methoxy-5,6,9,10-tetrahydropyrazino[1′,2′:1,5]pyrazolo[3,4-f]isoquinolin-7(8H)-one,
2-[3-(dimethylamino)phenyl]-5,6,9,10-tetrahydropyrazino[1′,2′:1,5]pyrazolo[3,4-q]isoquinolin-7(8H)-one,
2-(3-{[2-(1H-pyrrol-1-yl)ethyl]amino}propyl)-4,5-dihydro-2H-pyrazolo[3,4-f]isoquinoline-3-carboxylic acid trifluoroacetate,
2-[3-(benzyloxy)propyl]-8-quinolin-3-yl-4,5-dihydro-2H-pyrazolo[3,4-f]isoquinoline-3-carboxylic acid,
2-{3-[(4-butoxybenzyl)amino]propyl}-4,5-dihydro-2H-pyrazolo[3,4-f]isoquinoline-3-carboxylic acid trifluoroacetate,
2-(1,3-benzodioxol-5-yl)-5,6,9,10-tetrahydropyrazino[1′,2′:1,5]pyrazolo[3,4-f]isoquinolin-7(8H)-one,
2-[(E)-2-(2-fluorophenyl)ethenyl]-5,6,9,10-tetrahydropyrazino[1′,2′:1,5]pyrazolo[3,4-q]isoquinolin-7(8H)-one,
2-[(1E)-hex-1-enyl]-5,6,9,10-tetrahydropyrazino[1′,2′:1,5]pyrazolo[3,4-f]isoquinolin-7(8H)-one trifluoroacetate,
2-anilino-5,6,8,9,10,11-hexahydro-7H-[1,4]diazepino[1′,2′:1,5]pyrazolo[3,4-f]isoquinolin-7-one,
5,6,8,9,10,11-hexahydro-7H-[1,4]diazepino[1′,2′:1,5]pyrazolo[3,4-f]isoquinolin-7-one,
2-chloro-5,6,9,10-tetrahydropyrazino[1′,2′:1,5]pyrazolo[3,4-f]isoquinolin-7(8H)-one,
2-[(E)-2-(4-methoxyphenyl)ethenyl]-5,6,9,10-tetrahydropyrazino[1′,2′:1,5]pyrazolo[3,4-f]isoquinolin-7(8H)-one,
2-(methylthio)-5,6,9,10-tetrahydropyrazino[1′,2′:1,5]pyrazolo[3,4-f]isoquinolin-7(8H)-one,
2-{3-[(2-furylmethyl)amino]propyl}-4,5-dihydro-2H-pyrazolo[3,4-f]isoquinoline-3-carboxylic acid trifluoroacetate,
2-azepan-1-yl-5,6,9,10-tetrahydropyrazino[1′,2′:1,5]pyrazolo[3,4-f]isoquinolin-7(8H)-one trifluoroacetate,
2-(3,6-dihydropyridin-1 (2H)-yl)-5,6,9,10-tetrahydropyrazino[1′,2′:1,5]pyrazolo[3,4-f]isoquinolin-7(8H)-one trifluoroacetate,
9-methyl-5,6,9,10-tetrahydropyrazino[1′,2′:1,5]pyrazolo[3,4-f]isoquinolin-7(8H)-one,
2-(piperidin-3-ylmethyl)-4,5-dihydro-2H-pyrazolo[3,4-f]isoquinoline-3-carboxylic acid hydrochloride,
9-(chloromethyl)-5,6,9,10-tetrahydropyrazino[1′,2′:1,5]pyrazolo[3,4-f]isoquinolin-7(8H)-one,
2-[(4-methoxybenzyl)amino]-5,6,9,10-tetrahydropyrazino[1′,2′:1,5]pyrazolo[3,4-f]isoquinolin-7(8H)-one trifluoroacetate,
2-(3-([2-(1H-imidazol-4-yl)ethyl]amino}propyl)-4,5-dihydro-2H-pyrazolo[3,4-f]isoquinoline-3-carboxylic acid trifluoroacetate,
2-(benzylamino)-5,6,9,10-tetrahydropyrazino[1′,2′:1,5]pyrazolo[3,4-f]isoquinolin-7(8H)-one trifluoroacetate,
2-(methylthio)-5,6,8,9,10,11-hexahydro-7H-[1,4]diazepino[1′,2′:1,5]pyrazolo[3,4-f]isoquinolin-7-one,
2-{3-[(2-chlorobenzyl)amino]propyl}-4,5-dihydro-2H-pyrazolo[3,4-f]isoquinoline-3-carboxylic acid trifluoroacetate,
2-[3-(benzylamino)propyl]-4,5-dihydro-2H-pyrazolo[3,4-f]isoquinoline-3-carboxylic acid trifluoroacetate;
2-[3-({[(4-methoxyphenyl)amino]carbonyl}amino)propyl]-4,5-dihydro-2H-pyrazolo[3,4-f]isoquinoline-3-carboxylic acid,
2-{3-[(2-phenylethyl)amino]propyl}-4,5-dihydro-2H-pyrazolo[3,4-f]isoquinoline-3-carboxylic acid trifluoroacetate,
2-{3-[(thien-2-ylmethyl)amino]propyl}-4,5-dihydro-2H-pyrazolo[3,4-f]isoquinoline-3-carboxylic acid trifluoroacetate,
2-benzyl-5,6,9,10-tetrahydropyrazino[1′,2′:1,5]pyrazolo[3,4-f]isoquinolin-7(8H)-one,
5,6,8,9,10,11-hexahydro-7H-[1,4]diazepino[1′,2′:1,5]pyrazolo[3,4-f]isoquinolin-7-one,
2-{3-[(4-chlorobenzyl)amino]propyl}-4,5-dihydro-2H-pyrazolo[3,4-f]isoquinoline-3-carboxylic acid trifluoroacetate,
2-{3-[(2-phenylpropyl)amino]propyl}-4,5-dihydro-2H-pyrazolo[3,4-f]isoquinoline-3-carboxylic acid trifluoroacetate,
7-oxo-5,6,7,8,9,10-hexahydropyrazino[1′,2′:1,5]pyrazolo[3,4-f]isoquinoline-9-carboxamide trifluoroacetate,
2-(3-hydroxypropyl)-4,5-dihydro-2H-pyrazolo[3,4-f]isoquinoline-3-carboxylic acid,
2-(1,3-dihydro-2H-isoindol-2-yl)-5,6,9,10-tetrahydropyrazino[1′,2′:1,5]pyrazolo[3,4-f]isoquinolin-7(8H)-one trifluoroacetate,
2-{3-[(4-aminophenyl)amino]propyl}-4,5-dihydro-2H-pyrazolo[3,4-f]isoquinoline-3-carboxylic acid trifluoroacetate,
2-(4-hydroxypiperidin-1-yl)-5,6,9,10-tetrahydropyrazino[1′,2′:1,5]pyrazolo[3,4-f]isoquinolin-7(8H)-one trifluoroacetate,
2-(3-aminopropyl)-7-hydroxy-8-(3-nitrophenyl)-4,5-dihydro-2H-benzo[g]indazole-3-carboxylic acid trifluoroacetate,
2-(2-aminoethyl)-7-hydroxy-8-(3-nitrophenyl)-4,5-dihydro-2H-benzo[g]indazole-3-carboxylic acid trifluoroacetate,
3-hydroxy-2-(3-nitrophenyl)-5,6,8,9,10,11-hexahydro-7H-benzo[g][1,4]diazepino[1,2-b]indazol-7-one trifluoroacetate,
2-(3-aminopropyl)-7-hydroxy-4,5-dihydro-2H-benzo[g]indazole-3-carboxylic acid dihydrochloride,
2-(2-aminoethyl)-8-bromo-7-hydroxy-4,5-dihydro-2H-benzo[g]indazole-3-carboxylic acid trifluoroacetate,
2-(3-aminopropyl)-8-[2-(4-chlorophenyl)ethyl]-7-hydroxy-4,5-dihydro-2H-benzo[g]indazole-3-carboxylic acid trifluoroacetate,
3-hydroxy-2-(3-nitrophenyl)-5,6,9,10-tetrahydrobenzo[g]pyrazino[1,2-b]indazol-7(8H)-one hydrobromide,
2-(3-aminopropyl)-8-bromo-7-hydroxy-4,5-dihydro-2H-benzo[g]indazole-3-carboxylic acid hydrobromide,
2-(3-aminopropyl)-7-hydroxy-8-(4-nitrophenyl)-4,5-dihydro-2H-benzo[g]indazole-3-carboxylic acid trifluoroacetate,
2-(3-aminopropyl)-8-(3-cyanophenyl)-7-hydroxy-4,5-dihydro-2H-benzo[g]indazole-3-carboxylic acid trifluoroacetate,
2-(3-aminopropyl)-7-hydroxy-8-[3-(trifluoromethyl)phenyl]-4,5-dihydro-2H--benzo[g]indazole-3-carboxylic acid trifluoroacetate, and
2-(3-aminopropyl)-7-hydroxy-8-(3,3,3-trifluoropropyl)-4,5-dihydro-2H-benzo[g]indazole-3-carboxylic acid trifluoroacetate.

21. The cyclic pyrazole MK-2 inhibitor according to claim 1, wherein the MK-2 inhibitor has an MK-2 IC₅₀ of less than 1 and is selected from the group consisting of:
2-(3-aminopropyl)-8-quinolin-3-yl-4,5-dihydro-2H-pyrazolo[4,3-h]quinazoline-3-carboxylic acid dihydrochloride,
2-(3-aminopropyl)-8-(1,3-benzodioxol-5-yl)-4,5-dihydro-2H-pyrazolo[4,3-h]quinazoline-3-carboxylic acid hydrochloride,
2-(3-aminopropyl)-8-phenyl-4,5-dihydro-2H-pyrazolo[4,3-h]quinazoline-3-carboxylic acid hydrochloride,
2-(3-aminopropyl)-8-quinolin-3-yl-4,5-dihydro-2H-pyrazolo[3,4-f]isoquinoline-3-carboxylic acid trifluoroacetate,
2-(3-aminopropyl)-8-(3-nitrophenyl)-4,5-dihydro-2H-pyrazolo[3,4-f]isoquinoline-3-carboxylic acid trifluoroacetate,
2-(3-aminopropyl)-8-(4-hydroxyphenyl)-4,5-dihydro-2H-pyrazolo[3,4-f]isoquinoline-3-carboxylic acid hydrobromide,
2-(3-aminopropyl)-8-(3-hydroxyphenyl)-4,5-dihydro-2H-pyrazolo[3,4-f]isoquinoline-3-carboxylic acid hydrobromide,
2-(3-aminopropyl)-8-(2-naphthyl)-4,5-dihydro-2H-pyrazolo[3,4-f]isoquinoline-3-carboxylic acid trifluoroacetate,
2-(3-aminopropyl)-8-(3,5-difluorophenyl)-4,5-dihydro-2H-pyrazolo[3,4-f]isoquinoline-3-carboxylic acid trifluoroacetate,
2-(3-aminopropyl)-8-(1,3-benzodioxol-5-yl)-4,5-dihydro-2H-pyrazolo[3,4-f]isoquinoline-3-carboxylic acid trifluoroacetate,
2-(3-aminopropyl)-8-(3-cyanophenyl)-4,5-dihydro-2H-pyrazolo[3,4-f]isoquinoline-3-carboxylic acid trifluoroacetate,
9-(hydroxymethyl)-2-quinolin-3-yl-5,6,9,10-tetrahydropyrazino[1′,2′:1,5]pyrazolo[3,4-f]isoquinolin-7(8H)-one trifluoroacetate,
2-(3-aminopropyl)-8-(4-methoxyphenyl)-4,5-dihydro-2H-pyrazolo[3,4-f]isoquinoline-3-carboxylic acid trifluoroacetate,
2-(3-aminopropyl)-8-[3-(methylsulfonyl)phenyl]-4,5-dihydro-2H-pyrazolo[3,4-f]isoquinoline-3-carboxylic acid trifluoroacetate,
2-(3-aminopropyl)-8-[3-(trifluoromethyl) phenyl]-4,5-dihydro-2H-pyrazolo[3,4-f]isoquinoline-3-carboxylic acid trifluoroacetate,
2-(1H-imidazol-1-yl)-5,6,9,10-tetrahydropyrazino[1′,2′:1,5]pyrazolo[3,4-f]isoquinolin-7(8H)-one trifluoroacetate,
2-(3-aminopropyl)-8-(3-methoxyphenyl)-4,5-dihydro-2H-pyrazolo[3,4-f]isoquinoline-3-carboxylic acid trifluoroacetate,
2-(3-aminopropyl)-8-(4-(trifluoromethyl)phenyl]-4,5-dihydro-2H-pyrazolo[3,4-f]isoquinoline-3-carboxylic acid trifluoroacetate,
2-(3-aminopropyl)-8-anilino-4,5-dihydro-2H-pyrazolo[3,4-f]isoquinoline-3-carboxylic acid,
2-(3-aminopropyl)-8-(3,4-difluorophenyl)-4,5-dihydro-2H-pyrazolo[3,4-f]isoquinoline-3-carboxylic acid trifluoroacetate,
2-(3-{[2-(4′-carboxy-1,1′-biphenyl-4-yl)ethyl]amino}propyl)-4,5-dihydro-2H-pyrazolo[3,4-f]isoquinoline-3-carboxylic acid trifluoroacetate,
2-(4-hydroxyphenyl)-5,6,9,10-tetrahydropyrazino[1′,2′:1,5]pyrazolo[3,4-f]isoquinolin-7(8H)-one trifluoroacetate,
2-propyl-8-quinolin-3-yl-4,5-dihydro-2H-pyrazolo[3,4-f]isoquinoline-3-carboxylic acid,
2-[3-({2-[3′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
2-(3-aminopropyl)-8-(4-tert-butylphenyl)-4,5-dihydro-2H-pyrazolo[3,4-f]isoquinoline-3-carboxylic acid trifluoroacetate,
2-[3-({2-[4-(3-furyl)phenyl]ethyl}amino)propyl]-4,5-dihydro-2H-pyrazolo[3,4-f]isoquinoline-3-carboxylic acid trifluoroacetate,
2-(3-{[2-(3′-chloro-1,1′-biphenyl-4-yl)ethyl]amino}propyl)-4,5-dihydro-2H-pyrazolo[3,4-f]isoquinoline-3-carboxylic acid trifluoroacetate,
2-(4-methoxyphenyl)-5,6,8,9,10,11-hexahydro-7H-[1,4]diazepino[1′,2′:1,5]pyrazolo[3,4-f]isoquinolin-7-one,
2-[3-({2-[4′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
2-(3-hydroxyphenyl)-5,6,9,10-tetrahydropyrazino[1′,2′:1,5]pyrazolo[3,4-f]isoquinolin-7(8H)-one,
2-(3-aminopropyl)-N-hydroxy-8-quinolin-3-yl-4,5-dihydro-2H-pyrazolo[3,4-f]isoquinoline-3-carboxamide hydrochloride,
2-[(E)-2-(4-hydroxyphenyl)ethenyl]-5,6,9,10-tetrahydropyrazino[1′,2′:1,5]pyrazolo[3,4-f]isoquinolin-7(8H)-one trifluoroacetate,
2-quinolin-3-yl-5,6,8,9,10,11-hexahydro-7H-[1,4]diazepino[1′,2′:1,5]pyrazolo[3,4-f]isoquinolin-7-one,
2-(4-hydroxyphenyl)-5,6,8,9,10,11-hexahydro-7H-[1,4]diazepino[1′,2′:1,5]pyrazolo[3,4-f]isoquinolin-7-one,
2-(3-{[2-(2′-chloro-1,1′-biphenyl-4-yl)ethyl]amino}propyl)-4,5-dihydro-2H-pyrazolo[3,4-f]isoquinoline-3-carboxylic acid trifluoroacetate,
2-(3-{[2-(4′-tert-butyl-1,1′-biphenyl-4-yl)ethyl]amino}propyl)-4,5-dihydro-2H-pyrazolo[3,4-f]isoquinoline-3-carboxylic acid trifluoroacetate,
2-(3-{[2-(3,4-dichlorophenyl)ethyl]amino}propyl)-4,5-dihydro-2H-pyrazolo[3,4-f]isoquinoline-3-carboxylic acid trifluoroacetate,
2-(3-{[3-(3-chlorophenyl)propyl]amino}propyl)-4,5-dihydro-2H-pyrazolo[3,4-f]isoquinoline-3-carboxylic acid trifluoroacetate,
2-[(E)-2-phenylethenyl]-5,6,9,10-tetrahydropyrazino[1′,2′:1,5]pyrazolo[3,4-f]isoquinolin-7(8H)-one trifluoroacetate,
2-(3-{[2-(4-pyridin-4-ylphenyl)ethyl]amino}propyl)-4,5-dihydro-2H-pyrazolo[3,4-f]isoquinoline-3-carboxylic acid trifluoroacetate,
2-(3-{([3-(4-bromophenyl)propyl]amino}propyl)-4,5-dihydro-2H-pyrazolo[3,4-f]isoquinoline-3-carboxylic acid trifluoroacetate,
2-(3-{[3-(4-tert-butylphenyl)propyl]amino}propyl)-4,5-dihydro-2H-pyrazolo[3,4-f]isoquinoline-3-carboxylic acid trifluoroacetate,
2-(3-{[2-(3′-isopropyl-1,1′-biphenyl-4-yl)ethyl]amino}propyl)-4,5-dihydro-2H-pyrazolo[3,4-f]isoquinoline-3-carboxylic acid trifluoroacetate,
2-{3-[(2-thien-2-ylethyl)amino]propyl}-4,5-dihydro-2H-pyrazolo[3,4-f]isoquinoline-3-carboxylic acid,
9-(aminomethyl)-5,6,9,10-tetrahydropyrazino[1′,2′:1,5]pyrazolo[3,4-f]isoquinolin-7(8H)-one trifluoroacetate,
2-(3-nitrophenyl)-5,6,8,9,10,11-hexahydro-7H-[1,4]diazepino[1′,2′:1,5]pyrazolo[3,4-f]isoquinolin-7-one,
2-(3-aminopropyl)-8-quinolin-3-yl-4,5-dihydro-2H-pyrazolo[3,4-f]isoquinoline-3-carboxamide hydrochloride,
2-(3-{[3-(4-chlorophenyl)propyl]amino}propyl)-4,5-dihydro-2H-pyrazolo[3,4-f]isoquinoline-3-carboxylic acid trifluoroacetate,
2-[3-(dimethylamino)phenyl]-5,6,8,9,10,11-hexahydro-7H-[1,4]diazepino[1′,2′:1,5]pyrazolo[3,4-f]isoquinolin-7-one,
2-(3-{[(4-chlorobenzyl)sulfonyl]amino}propyl)-8-quinolin-3-yl-4,5-dihydro-2H-pyrazolo[3,4-f]isoquinoline-3-carboxylic acid,
2-(3-{[2-(4-pyridin-3-ylphenyl)ethyl]amino}propyl)-4,5-dihydro-2H-pyrazolo[3,4-f]isoquinoline-3-carboxylic acid trifluoroacetate,
2-(3-{[2-(4-chlorophenyl)ethyl]amino}propyl)-4,5-dihydro-2H-pyrazolo[3,4-f]isoquinoline-3-carboxylic acid trifluoroacetate,
2-(3-{[2-(5-chlorothien-2-yl)ethyl]amino}propyl)-4,5-dihydro-2H-pyrazolo[3,4-f]isoquinoline-3-carboxylic acid trifluoroacetate,
2-(3-{[3-(4-cyanophenyl)propyl]amino}propyl)-4,5-dihydro-2H-pyrazolo[3,4-f]isoquinoline-3-carboxylic acid trifluoroacetate,
2-(3-{[3-(5-methyl-2-furyl)butyl]amino}propyl)-4,5-dihydro-2H-pyrazolo[3,4-f]isoquinoline-3-carboxylic acid trifluoroacetate,
2-[3-({2-[4-(1-benzothien-3-yl)phenyl]ethyl}amino)propyl]-4,5-dihydro-2H-pyrazolo[3,4-f]isoquinoline-3-carboxylic acid trifluoroacetate,
2-(3-ammoniopropyl)-3-carboxy-4,5-dihydro-2H-pyrazolo[3,4-f]isoquinolin-7-ium dichloride,
2-(4-methoxyphenyl)-5,6,9,10-tetrahydropyrazino[1′,2′:1,5]pyrazolo[3,4-f]isoquinolin-7(8H)-one,
2-(3-{[3-(4-acetylphenyl)propyl]amino}propyl)-4,5-dihydro-2H-pyrazolo[3,4-f]isoquinoline-3-carboxylic acid trifluoroacetate,
2-(3-aminopropyl)-4,5-dihydro-2H-pyrazolo[3,4-f]isoquinoline-3-carboxylic acid trifluoroacetate,
2-[3-({3-[4-(methylsulfonyl)phenyl]propyl}amino)propyl]-4,5-dihydro-2H-pyrazolo[3,4-f]isoquinoline-3-carboxylic acid trifluoroacetate,
2-(3-aminopropyl)-8-(2-methoxyphenyl)-4,5-dihydro-2H-pyrazolo[3,4-f]isoquinoline-3-carboxylic acid trifluoroacetate,
2-[3-({2-[3′-(aminomethyl)-1,1′-biphenyl-4-yl]ethyl}amino)propyl]-4,5-dihydro-2H-pyrazolo[3,4-f]isoquinoline-3-carboxylic acid trifluoroacetate,
2-(2-aminoethyl)-4,5-dihydro-2H-pyrazolo[3,4-f]isoquinoline-3-carboxylic acid dihydrochloride,
2-(3-{[2-(4-nitrophenyl)ethyl]amino}propyl)-4,5-dihydro-2H-pyrazolo[3,4-f]isoquinoline-3-carboxylic acid trifluoroacetate,
2-[3-({2-[2′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate,
9-(hydroxymethyl)-5,6,9,10-tetrahydropyrazino[1′,2′:1,5]pyrazolo[3,4-f]isoquinolin-7(8H)-one,
2-(3-{[2-(4-methylphenyl)ethyl]amino}propyl)-4,5-dihydro-2H-pyrazolo[3,4-f]isoquinoline-3-carboxylic acid trifluoroacetate,
9-{[(2-thien-2-ylethyl)amino]methyl}-5,6,9,10-tetrahydropyrazino[1′,2′:1,5]pyrazolo[3,4-f]isoquinolin-7(8H)-one,
2-(3-{[2-(4-ethoxyphenyl)ethyl]amino}propyl)-4,5-dihydro-2H-pyrazolo[3,4-f]isoquinoline-3-carboxylic acid trifluoroacetate,
2-(1,3-benzodioxol-5-yl)-5,6,8,9,10,11-hexahydro-7H-[1,4]diazepino[1′,2′:1,5]pyrazolo[3,4-f]isoquinolin-7-one,
2-(3-{[2-(4-methoxyphenyl)ethyl]amino}propyl)-4,5-dihydro-2H-pyrazolo[3,4-f]isoquinoline-3-carboxylic acid trifluoroacetate,
2-(3-aminopropyl)-7-hydroxy-8-(3-nitrophenyl)-4,5-dihydro-2H-benzo[g]indazole-3-carboxylic acid trifluoroacetate, and
2-(2-aminoethyl)-7-hydroxy-8-(3-nitrophenyl)-4,5-dihydro-2H-benzo[g]indazole-3-carboxylic acid trifluoroacetate.

22. A method of inhibiting mitogen activated protein kinase-activated protein kinase-2 in a subject in need of such inhibition, the method comprising administering to the subject an MK-2 inhibiting compound having the structure shown in claim 1.

23. The method according to claim 22, wherein the MK-2 inhibiting compound is administered in an effective amount.

24. The method according to claim 22, wherein the MK-2 inhibiting compound has the structure shown in claim 2.

25. A method of preventing or treating a TNFα mediated disease or disorder in a subject in need of such prevention or treatment, the method comprising administering to the subject an effective amount of an MK-2 inhibiting compound having the structure shown in claim 1.

26. The method according to claim 25, wherein the MK-2 inhibiting compound has the structure shown in claim 2.

27. The method according to claim 25, wherein the subject is a mammal.

28. The method according to claim 27, wherein the subject is a human.

29. The method according to claim 25, wherein the TNFα mediated disease or disorder is selected from the group consisting of: arthritis, rheumatoid arthritis, spondyloarthopathies, gouty arthritis, osteoarthritis, systemic lupus erythematosus, juvenile arthritis, asthma, bronchitis, menstrual cramps, tendinitis, bursitis, connective tissue injuries or disorders, skin related conditions, psoriasis, eczema, burns, dermatitis, gastrointestinal conditions, inflammatory bowel disease, gastric ulcer, gastric varices, Crohn's disease, gastritis, irritable bowel syndrome, ulcerative colitis, Cancer, colorectal cancer, herpes simplex infections, HIV, pulmonary edema, kidney stones, minor injuries, wound healing, vaginitis, candidiasis, lumbar spondylanhrosis, lumbar spondylarthrosis, vascular diseases, migraine headaches, sinus headaches, tension headaches, dental pain, periarteritis nodosa, thyroiditis, aplastic anemia, Hodgkin's disease, sclerodoma, rheumatic fever, type I diabetes, myasthenia gravis, multiple sclerosis, sarcoidosis, nephrotic syndrome, Behcet's syndrome, polymyositis, gingivitis, hypersensitivity, swelling occurring after injury, myocardial ischemia, ophthalmic diseases, retinitis, retinopathies, conjunctivitis, uveitis, ocular photophobia, acute injury to the eye-tissue, pulmonary inflammation, viral infections, cystic fibrosis, Central nervous system disorders, cortical dementias, and Alzheimer's disease.

30. The method according to claim 25, wherein the subject is administered an effective amount of the MK-2 inhibiting compound.

31. The method according to claim 22, wherein the MK-2 inhibiting compound provides an MK-2 inhibition IC₅₀ value of below 50 μM.

32. The method according to claim 22, wherein the MK-2 inhibiting compound provides an MK-2 inhibition IC₅₀ value of below 20 μM.

33. The method according to claim 22, wherein the MK-2 inhibiting compound provides an MK-2 inhibition IC₅₀ value of below 10 μM.

34. The method according to claim 22, wherein the MK-2 inhibiting compound provides an MK-2 inhibition IC₅₀ value of below 1 μM.

35. The method according to claim 22, wherein the MK-2 inhibiting compound provides a selectivity ratio (IC_{50 MK-3}/IC_{50 MK-2}) of at least about 1.

36. The method according to claim 22, wherein the MK-2 inhibiting compound provides a selectivity ratio (IC_{50 MK-3}/IC_{50 MK-2}) of at least about 10.

37. The method according to claim 22, wherein the MK-2 inhibiting compound provides a selectivity ratio (IC_{50 MK-3}/IC_{50 MK-2}) of at least about 100.

38. A pharmaceutical composition comprising an MK-2 inhibiting compound comprising a pharmaceutical carrier and an MK-2 inhibiting compound having the structure shown in claim 1.

39. A kit comprising a dosage form that includes an MK-2 inhibiting compound in a therapeutically effective amount, the MK-2 inhibiting compound having the structure shown in claim 1.

40. The cyclic pyrazole MK-2 inhibitor according to claim 1, wherein the compound is selected from the group consisting of:
2-[(E)-2-(3-hydroxyphenyl)ethenyl]-5,6,9,10-tetrahydropyrazino[1′,2′:1,5]pyrazolo[3,4-f]isoquinolin-7(8H)-one trifluoroacetate,
2-[(E)-2-phenylethenyl]-5,6,9,10-tetrahydropyrazino[1′,2′:1,5]pyrazolo[3,4-f]isoquinolin-7(8H)-one trifluoroacetate,
2-[(E)-2-(4-hydroxyphenyl)ethenyl]-5,6,9,10-tetrahydropyrazino[1′,2′:1,5]pyrazolo[3,4-f]isoquinolin-7(8H)-one trifluoroacetate,
2-[(E)-2-(1,3-benzodioxol-5-yl)ethenyl]-5,6,9,10-tetrahydropyrazino[1′,2′:1,5]pyrazolo[3,4-f]isoquinolin-7(8H)-one,
2-[(E)-2-(3-methoxyphenyl)ethenyl]-5,6,9,10-tetrahydropyrazino[1′,2′:1,5]pyrazolo[3,4-f]isoquinolin-7(8H)-one trifluoroacetate,
2-[(E)-2-(4-chlorophenyl)ethenyl]-5,6,9,10-tetrahydropyrazino[1′,2′:1,5]pyrazolo[3,4-f]isoquinolin-7(8H)-one hydrochloride,
2-[(E)-2-(3,4-difluorophenyl)ethenyl]-5,6,9,10-tetrahydropyrazino[1′,2′:1,5]pyrazolo[3,4-f]isoquinolin-7(8H)-one,
2-[(E)-2-(4-fluorophenyl)ethenyl]-5,6,9,10-tetrahydropyrazino[1′,2′:1,5]pyrazolo[3,4-f]isoquinolin-7(8H)-one,
2-{(E)-2-[4-(trifluoromethyl)phenyl]ethenyl}-5,6,9,10-tetrahydropyrazino[1′,2′:1,5]pyrazolo[3,4-f]isoquinolin-7(8H)-one,
2-[(E)-2-(3-chlorophenyl)ethenyl]-5,6,9,10-tetrahydropyrazino[1′,2′:1,5]pyrazolo[3,4-f]isoquinolin-7(8H)-one,
2-[(E)-2-(2-fluorophenyl)ethenyl]-5,6,9,10-tetrahydropyrazino[1′,2′:1,5]pyrazolo[3,4-f]isoquinolin-7(8H)-one,
2-[(E)-2-(4-methoxyphenyl)vinyl]-5,6,9,10-tetrahydropyrazino[1′,2′:1,5]pyrazolo[3,4-f]isoquinolin-7(8H)-one,
2-[(E)-2-phenylethenyl]-5,6,9,10-tetrahydropyrazino[1′,2′:1,5]pyrazolo[3,4-f]isoquinolin-7(8H)-one.,
2-[(E)-2-(4-methoxyphenyl)vinyl]-5,6,9,10-tetrahydropyrazino[1′,2′:1,5]pyrazolo[3,4-f]isoquinolin-7(8H)-one trifluoroacetate,
2-[(E)-2-(4-chlorophenyl)ethenyl]-5,6,9,10-tetrahydropyrazino[1′,2′:1,5]pyrazolo[3,4-f]isoquinolin-7(8H)-one,
2-[(E)-2-(4-methylphenyl)ethenyl]-5,6,9,10-tetrahydropyrazino[1′,2′:1,5]pyrazolo[3,4-f]isoquinolin-7(8H)-one,
2-[(E)-2-(4-methylphenyl)ethenyl]-5,6,9,10-tetrahydropyrazino[1′,2′:1,5]pyrazolo[3,4-f]isoquinolin-7(8H)-one hydrochloride,
ethyl 3-[(E)-2-(7-oxo-5,6,7,8,9,10-hexahydropyrazino[1′,2′:1,5]pyrazolo[3,4-f]isoquinolin-2-yl)ethenyl]benzoate,
9-(hydroxymethyl)-2-[(E)-2-phenylvinyl]-5,6,9,10-tetrahydropyrazino[1′,2′:1,5]pyrazolo[3,4-f]isoquinolin-7(8H)-one,
3-[(E)-2-(7-oxo-5,6,7,8,9,10-hexahydropyrazino[1′,2′:1,5]pyrazolo[3,4-f]isoquinolin-2-yl)vinyl]benzoic acid,
lithium 3-[(E)-2-(7-oxo-5,6,7,8,9,10-hexahydropyrazino[1′,2′:1,5]pyrazolo[3,4-f]isoquinolin-2-yl)vinyl]benzoate,
methyl 7-oxo-2-[(E)-2-phenylvinyl]-5,6,7,8,9,10-hexahydropyrazino[1′,2′:1,5]pyrazolo[3,4-f]isoquinoline-9-carboxylate,
ethyl 4-[(E)-2-(7-oxo-5,6,7,8,9,10-hexahydropyrazino[1′,2′:1,5]pyrazolo[3,4-f]isoquinolin-2-yl)vinyl]benzoate,
4-[(E)-2-(7-oxo-5,6,7,8,9,10-hexahydropyrazino[1′,2′:1,5]pyrazolo[3,4-f]isoquinolin-2-yl)vinyl]benzoic acid,
2-((E)-2-{4-[(4-methylpiperazin-1-yl)carbonyl]phenyl}vinyl)-5,6,9,10-tetrahydropyrazino[1′,2′:1,5]pyrazolo[3,4-f]isoquinolin-7(8H)-one trifluoroacetate,
2-{(E)-2-[4-(pyrrolidin-1-ylcarbonyl)phenyl]vinyl}-5,6,9,10-tetrahydropyrazino[1′,2′:1,5]pyrazolo[3,4-f]isoquinolin-7(8H)-one trifluoroacetate,
2-{(E)-2-[4-(morpholin-4-ylcarbonyl)phenyl]vinyl}-5,6,9,10-tetrahydropyrazino[1′,2′:1,5]pyrazolo[3,4-f]isoquinolin-7(8H)-one trifluoroacetate,
2-[(E)-2-(4-{[(2S)-2-(pyrrolidin-1-ylmethyl)pyrrolidin-1-yl]carbonyl}phenyl)vinyl]-5,6,9,10-tetrahydropyrazino[1′,2′:1,5]pyrazolo[3,4-f]isoquinolin-7(8H)-one trifluoroacetate,
2-[(E)-2-(3-{[(2R)-2-(pyrrolidin-1-ylmethyl)pyrrolidin-1-yl]carbonyl}phenyl)vinyl]-5,6,9,10-tetrahydropyrazino[1′,2′:1,5]pyrazolo[3,4-f]isoquinolin-7(8H)-one trifluoroacetate,
2-((E)-2-{3-[(4-methylpiperazin-1-yl)carbonyl]phenyl}vinyl)-5,6,9,10-tetrahydropyrazino[1′,2′:1,5]pyrazolo[3,4-f]isoquinolin-7(8H)-one trifluoroacetate,
N,N-dimethyl-3-[(E)-2-(7-oxo-5,6,7,8,9,10-hexahydropyrazino[1′,2′:1,5]pyrazolo[3,4-f]isoquinolin-2-yl)vinyl]benzamide trifluoroacetate,
2-[(E)-2-phenylvinyl]-5,6,8,9,10,11-hexahydro-7H-[1,4]diazepino[1′,2′:1,5]pyrazolo[3,4-f]isoquinolin-7-one,
2-(3-aminopropyl)-8-[(E)-2-phenylvinyl]-4,5-dihydro-2H-pyrazolo[3,4-f]isoquinoline-3-carboxylic acid trifluoroacetate,
2-(3-{[2-(4-bromophenyl)ethyl]amino}propyl)-8-[(E)-2-phenylvinyl]-4,5-dihydro-2H-pyrazolo[3,4-f]isoquinoline-3-carboxylic acid trifluoroacetate,
8-[(E)-2-phenylvinyl]-2-{3-[(2-{4-[(E)-2-phenylvinyl]phenyl}ethyl)amino]propyl}-4,5-dihydro-2H-pyrazolo[3,4-f]isoquinoline-3-carboxylic acid trifluoroacetate,
ethyl 2-(3-aminopropyl)-8-[(E)-2-phenylvinyl]-4,5-dihydro-2H-pyrazolo[3,4-f]isoquinoline-3-carboxylate hydrochloride,
2-(2-aminoethyl)-8-[(E)-2-phenylethenyl]-4,5-dihydro-2H-pyrazolo[3,4-f]isoquinoline-3-carboxylic acid hydrochloride, and
ethyl 2-(2-aminoethyl)-8-[(E)-2-phenylethenyl]-4,5-dihydro-2H-pyrazolo[3,4-f]isoquinoline-3-carboxylate hydrochloride.
